(19) United States
(12) Patent Application Publication
(10) Pub. No.: US 2025/0269002 A1
(43) Pub. Date: Aug. 28, 2025

(54) CTA VACCINE CASSETTES

(71) Applicant: Gritstone Bio, Inc., Emeryville, CA (US)

(72) Inventors: Karin Jooss, San Diego, CA (US); Roman Yelensky, Newton, MA (US); James Xin Sun, Newton, MA (US); Amy Rachel Rappaport, Daly City, CA (US); Ciaran Daniel Scallan, San Francisco, CA (US); Leonid Gitlin, Foster City, CA (US); Christine Denise Palmer, Cambridge, MA (US); Melissa Rotunno, Southborough, MA (US); Monica Lane, Uxbridge, MA (US)

(21) Appl. No.: 19/044,124

(22) Filed: Feb. 3, 2025

Related U.S. Application Data
(63) Continuation of application No. PCT/US2023/071623, filed on Aug. 3, 2023.
(60) Provisional application No. 63/370,364, filed on Aug. 3, 2022.

(51) Int. Cl. A61K 39/00; A61K 9/00; A61P 35/00; C12N 15/86

(52) U.S. Cl. CPC A61K 39/001186; A61K 9/0019; A61K 39/001164; A61P 35/00; C12N 15/86; A61K 2039/53; A61K 2039/54; A61K 2039/545; A61K 2039/55555; A61K 2039/572; A61K 2039/70; C12N 2710/10334; C12N 2710/10343; C12N 2710/10351; C12N 2830/003; C12N 2830/50

(57) ABSTRACT

Disclosed herein are compositions that include antigen-encoding nucleic acid sequences having multiple iterations of CTA epitope-encoding sequences or Cancer Testis Antigen (CTA)-encoding nucleic acid sequences and KRAS-encoding nucleic acid sequences. Also disclosed are nucleotides, cells, and methods associated with the compositions including their use as vaccines.

### CROSS REFERENCE TO RELATED APPLICATIONS

This application is a continuation of International Patent Application No PCT/US2023/071623, filed Aug. 3, 2023, which claims the benefit of U.S. Provisional Application No. 63/370,364, filed Aug. 3, 2022, which is hereby incorporated in its entirety by reference for all purposes.

### SEQUENCE LISTING

The instant application contains a Sequence Listing which has been submitted electronically and is hereby incorporated herein by reference in its entirety. Said .XML copy, created on Nov. 10, 2022, is named GSO-086WOC1 and is 400,000 bytes in size.

### BACKGROUND

Therapeutic vaccines based on tumor-specific antigens hold great promise as a next-generation of personalized cancer immunotherapy.¹⁻³ For example, cancers with a high mutational burden, such as non-small cell lung cancer (NSCLC) and melanoma, are particularly attractive targets of such therapy given the relatively greater likelihood of neoantigen generation.^{4,5} Early evidence shows that neoantigen-based vaccination can elicit T-cell responses⁶ and that neoantigen targeted cell-therapy can cause tumor regression under certain circumstances in selected patients.⁷

One question for antigen vaccine design in both cancer and infectious disease settings is which of the many coding mutations present generate the “best” therapeutic antigens, e.g., antigens that can elicit immunity.

In addition to the challenges of current antigen prediction methods certain challenges also exist with the available vector systems that can be used for antigen delivery in humans, many of which are derived from humans. For example, many humans have pre-existing immunity to human viruses as a result of previous natural exposure, and this immunity can be a major obstacle to the use of recombinant human viruses for antigen delivery in vaccination strategies, such as in cancer treatment or vaccinations against infectious diseases. While some progress has been made in vaccinations strategies addressing the above problems, improvements are still needed, particularly for clinical applications, such as improved vaccine potency and efficacy.

### SUMMARY

Disclosed herein is an antigen-encoding vaccine system, wherein the antigen-encoding vaccine system comprises: (a) a Cancer Testis Antigen (CTA)-encoding nucleic acid sequence encoding a CTA-associated MHC class I epitope; and (b) a KRAS-encoding nucleic acid sequence encoding a KRAS-associated MHC class I epitope.

In some aspects, the CTA-encoding nucleic acid sequence and the KRAS-encoding nucleic acid sequence are encoded in a single cassette. In some aspects, the CTA-encoding nucleic acid sequence and the KRAS-encoding nucleic acid sequence are encoded on separate vectors. In some aspects, the system comprises a mixture of the separate vectors.

In some aspects, (a) the system comprises two or more iterations of the CTA-encoding nucleic acid sequence; or (b) the system comprises two or more iterations of the KRAS-encoding nucleic acid sequence; or (c) the system comprises two or more iterations of each of the CTA-encoding nucleic acid sequence and the KRAS-encoding nucleic acid sequence, and wherein each iteration of the CTA-encoding nucleic acid sequence and/or the KRAS-encoding nucleic acid sequence, respectively, comprises identical nucleic acid sequences.

In some aspects, the CTA-encoding nucleic acid sequence comprises two or more distinct CTA-encoding nucleic acid sequences, wherein each distinct CTA-encoding nucleic acid sequence encodes a non-identical CTA-associated MHC class I epitope. In some aspects, the KRAS-encoding nucleic acid sequence comprises two or more distinct KRAS-encoding nucleic acid sequences, wherein each distinct KRAS-encoding nucleic acid sequence encodes a non-identical KRAS-associated MHC class I epitope. In some aspects, the CTA-encoding nucleic acid sequence comprises two or more distinct CTA-encoding nucleic acid sequences, wherein each distinct CTA-encoding nucleic acid sequence encodes a non-identical CTA-associated MHC class I epitope and the KRAS-encoding nucleic acid sequence comprises two or more distinct KRAS-encoding nucleic acid sequences, wherein each distinct KRAS-encoding nucleic acid sequence encodes a non-identical KRAS-associated MHC class I epitope. In some aspects, (a) the system comprises two or more iterations of at least one or each of the CTA-encoding nucleic acid sequences; or (b) the system comprises two or more iterations of at least one or each of the KRAS-encoding nucleic acid sequences; or (c) the system comprises two or more iterations of at least one or each of the CTA-encoding nucleic acid sequences and at least one or each of the KRAS-encoding nucleic acid sequences, and wherein each iteration of the CTA-encoding nucleic acid sequence and/or the KRAS-encoding nucleic acid sequence, respectively, comprises identical nucleic acid sequences.

Also disclosed herein is an antigen-encoding vaccine system, wherein the antigen-encoding vaccine system comprises: (i) a CTA-encoding nucleic acid sequence A (E_{A}); and (ii) a KRAS-encoding nucleic acid sequence B (E_{B}), wherein E_{A} and E_{B} each encode one MHC epitope, wherein E_{A} encodes a CTA-associated MHC class I epitope, and wherein E_{B} encodes a KRAS-associated MHC class I epitope.

In some aspects, the antigen-encoding vaccine system comprises: (a) a nucleic acid sequence comprising at least two iterations of E_{A}; or (b) a nucleic acid sequence comprising at least two iterations of E_{B}; or (c) a nucleic acid sequence comprising at least two iterations of E_{A} and a nucleic acid sequence comprising at least two iterations of E_{B}; or (d) a nucleic acid sequence comprising at least two iterations of E_{A} and at least two iterations of E_{B}, and wherein each iteration of E_{A} and/or E_{B}, respectively, comprises identical nucleic acid sequences.

In some aspects, E_{A} and E_{B} are encoded in a single cassette. In some aspects, E_{A} and E_{B} are encoded on separate vectors. In some aspects, the system comprises at least 2, at least 3, at least 4, at least 5, at least 6, at least 7, or at least 8 iterations of one or both of E_{A} and E_{B}. In some aspects, the system comprises at least 2 iterations of E_{A}. In some aspects, the system comprises at least 2 iterations of E_{B}. In some aspects, the system comprises at least 4 iterations of E_{B}. In some aspects, the system comprises at least 2 iterations of E_{A} and at least 2 iterations of E_{B}. In some aspects, the system comprises at least 2 iterations of E_{A} and at least 4 iterations of E_{B}.

In some aspects, the antigen-encoding cassette further comprises a nucleic acid sequence C (E_{C}), wherein E_{C} encodes one MHC epitope, wherein the MHC epitope encoded by E_{C} is and distinct and non-identical with respect to the MHC epitope encoded by E_{A} and the MHC epitope encoded by E_{B}. In some aspects, E_{C} encodes a non-identical CTA-associated MHC class I epitope with respect to the MHC epitope encoded by E_{A} or a non-identical KRAS-associated MHC class I epitope with respect to the MHC epitope encoded by E_{B}.

Also disclosed herein is an antigen-encoding vaccine system, wherein the antigen-encoding vaccine system comprises: one or more vectors each comprising: (a) a vector backbone, wherein the backbone comprises: (i) at least one promoter nucleotide sequence, and (ii) at least one polyadenylation (poly(A)) sequence; and (b) a cassette, wherein the cassette comprises at least one antigen-encoding nucleic acid sequence, comprising at least 2 CTA-encoding nucleic acid sequences each encoding a distinct, non-identical CTA-associated MHC class I epitope, wherein each of the CTA-encoding nucleic acid sequences optionally comprises a 5′ linker sequence and/or a 3′ linker sequence, optionally wherein at least one of the CTA-encoding nucleic acid sequences comprises two or more iterations, wherein each iteration of the CTA-encoding nucleic acid sequence comprises an identical nucleic acid sequence.

Also disclosed herein is an antigen-encoding vaccine system, wherein the antigen-encoding vaccine system comprises: one or more vectors each comprising: (a) a vector backbone, wherein the backbone comprises: (i) at least one promoter nucleotide sequence, and (ii) at least one polyadenylation (poly(A)) sequence; and (b) a cassette, wherein the cassette comprises at least one antigen-encoding nucleic acid sequence, comprising at least one CTA-encoding nucleic acid sequence encoding a CTA-associated MHC class I epitope, wherein at least one of the at least one CTA-encoding nucleic acid sequences comprises two or more iterations, wherein each iteration of the CTA-encoding nucleic acid sequence comprises an identical nucleic acid sequence, and wherein the CTA-encoding nucleic acid sequences optionally comprises a 5′ linker sequence and/or a 3′ linker sequence, optionally comprising at least 2 CTA-encoding nucleic acid sequences each encoding a distinct, non-identical CTA-associated MHC class I epitope.

In some aspects, at least one or each of the CTA-associated MHC class I epitopes is selected from the group consisting of: a MAGEA1 MHC class I epitope encoding nucleic acid sequence, a MAGEA3 MHC class I epitope encoding nucleic acid sequence, a MAGEA4 MHC class I epitope encoding nucleic acid sequence, a MAGEA6 MHC class I epitope encoding nucleic acid sequence, a MAGEA8 MHC class I epitope encoding nucleic acid sequence, a MAGEA11 MHC class I epitope encoding nucleic acid sequence, a MAGEB2 MHC class I epitope encoding nucleic acid sequence, a CTCFL MHC class I epitope encoding nucleic acid sequence, and a CT83 MHC class I epitope encoding nucleic acid sequence. In some aspects, at least one or each of the CTA-associated MHC class I epitopes is selected from the group consisting of: FVQENYLEY, EVDPTSHSY, NTDNNLAVY, EVDPIGHLY, GVYDGREHTV, ALREEGEGV, KVLEYVIKV, GVYDGEEHSV, KLVELEHTL, AETSYVKVL, KVLEHVVRV, EADPTGHSY, SALPTTISF, GVYDGREHTVY, TQHFVQENY, EYVIKVSAR, LVRPSSSGL, GEMSSNSTAL, TVYGEPRKL, ALAETSYVK, TSYVKVLEH, YPSLREAAL, ALLEEEEGV, GPRQSLQQC, IAYPSLREAAL, and MEVDPIGHL.

In some aspects, the CTA-encoding nucleic acid sequence encodes:
each of a MAGEA1 MHC class I epitope encoding nucleic acid sequence, a MAGEA4 MHC class I epitope encoding nucleic acid sequence, a MAGEB2 MHC class I epitope encoding nucleic acid sequence, a MAGEA11 MHC class I epitope encoding nucleic acid sequence, a MAGEA3 MHC class I epitope encoding nucleic acid sequence, and a CT83 MHC class I epitope encoding nucleic acid sequence,
each of a MAGEA6 MHC class I epitope encoding nucleic acid sequence, a MAGEA11 MHC class I epitope encoding nucleic acid sequence, a MAGEA3 MHC class I epitope encoding nucleic acid sequence, and a CT83 MHC class I epitope encoding nucleic acid sequence,
each of a MAGEA1 MHC class I epitope encoding nucleic acid sequence, a MAGEA4 MHC class I epitope encoding nucleic acid sequence, a MAGEB2 MHC class I epitope encoding nucleic acid sequence, a MAGEA11 MHC class I epitope encoding nucleic acid sequence, and a CT83 MHC class I epitope encoding nucleic acid sequence, or
each of a MAGEA3 MHC class I epitope encoding nucleic acid sequence, a MAGEA4 MHC class I epitope encoding nucleic acid sequence, a MAGEB2 MHC class I epitope encoding nucleic acid sequence, a MAGEA11 MHC class I epitope encoding nucleic acid sequence, and a CT83 MHC class I epitope encoding nucleic acid sequence.

In some aspects, the CTA-encoding nucleic acid sequence comprises each of a CT83 MHC class I epitope encoding nucleic acid sequence, a MAGEA4 MHC class I epitope encoding nucleic acid sequence, a MAGEA1 MHC class I epitope encoding nucleic acid sequence, a MAGEA8, a MAGEB2 MHC class I epitope encoding nucleic acid sequence, a MAGEA3 MHC class I epitope encoding nucleic acid sequence, and a MAGEA11 MHC class I epitope encoding nucleic acid sequence. In some aspects, the CTA-encoding nucleic acid sequence encodes each of the CTA-associated MHC class I epitopes NTDNNLAVY, LVRPSSSGL, GEMSSNSTAL, GVYDGREHTV, GVYDGREHTVY, ALAETSYVK, KVLEHVVRV, SALPTTISF, GVYDGEEHSV, KVLEYVIKV, AETSYVKVL, EYVIKVSAR, EVDPIGHLY, MEVDPIGHL, and EVDPTSHSY. In some aspects, the CTA-encoding nucleic acid sequence encodes the amino acid sequence

ELGVMGVYDGREHTVYGEPRKELGVMGVYDGREHTVYGEPRKGID
VKEVDPTSHSYVLVTSGPRALAETSYVKVLEHVVRVNARVRGPRA
LAETSYVKVLEYVIKVSARVRFFFFLNMLGVYDGEEHSVFGEPWF
QRNTGEMSSNSTALALVRPSSSGLINSNTDNNLA VYDLSRDSPQ
GASALPTTISFTCWRQGIELMEVDPIGHLYIFATCFQRNTGEMSS
NSTALALVRPSSSGLINSNTDNNLAVYDLSRDSPQGASALPTTIS
FTCWRQGIELMEVDPIGHLYIFATCGPRALAETSYVKVLEHVVRV
NARVRGPRALAETSYVKVLEYVIKVSARVRFFFFLNMLGVYDGEE
HSVFGEPWGIDVKEVDPTSHSYVLVTS.

In some aspects, the CTA-encoding nucleic acid sequence comprises each of a CT83 MHC class I epitope encoding nucleic acid sequence, a MAGEA4 MHC class I epitope encoding nucleic acid sequence, a MAGEA1 MHC class I epitope encoding nucleic acid sequence, a MAGEA8, a MAGEA6 MHC class I epitope encoding nucleic acid sequence, a CTCFL MHC class I epitope encoding nucleic acid sequence, and a MAGEA3 MHC class I epitope encoding nucleic acid sequence. In some aspects, the CTA-encoding nucleic acid sequence encodes each of the CTA-associated MHC class I epitopes NTDNNLAVY, LVRPSSSGL, GEMSSNSTAL, KLVELEHTL, GVYDGREHTV, GVYDGREHTVY, ALAETSYVK, KVLEHVVRV, ALLEEEEGV, YPSLREAAL, IAYPSLREAAL, AETSYVKVL, FVQENYLEY, EVDPIGHLY, MEVDPIGHL, and GPRQSLQQC. In some aspects, the CTA-encoding nucleic acid sequence encodes the amino acid sequence

GPRALAETSYVKVLEHVVRVNARVRIA YPSLREAALLEEEEGVW
LEEGPRQSLQQCVAISLLTQYFVQENYLEYRQVPGMVENKLVELE
HTLLSKGIELMEVDPIGHLYIFATCQRNTGEMSSNSTALALVRPS
SSGLINSNTDNNLAVYDLSRWEELGVMGVYDGREHTVYGEPRKLL
TQDQRNTGEMSSNSTALALVRPSSSGLINSNTDNNLAVYDLSRWE
ELGVMGVYDGREHTVYGEPRKLLTQDWMVENKLVELEHTLLSKGI
ELMEVDPIGHLYIFATCPRALAETSYVKVLEHVVRVNARVRIAYP
SLREAALLEEEEGVWLEEGPRQSLQQCVAISLLTQYFVQENYLEY
RQVPG.

In some aspects, at least one or each of the KRAS-associated MHC class I epitopes comprises a neoepitope independently comprising a KRAS G12C mutation, a KRAS G12V mutation, a KRAS G12D mutation, or a KRAS Q61H mutation. In some aspects, at least one or each of the KRAS-associated MHC class I epitopes comprises a KRAS neoepitope independently comprising a KRAS G12C mutation or a KRAS G12V mutation. In some aspects, the KRAS-encoding nucleic acid sequence encodes each of a KRAS neoepitope comprising a G12C mutation and a KRAS neoepitope comprising a KRAS G12V mutation. In some aspects, the KRAS-encoding nucleic acid sequence encodes each of a KRAS neoepitope comprising a KRAS G12C mutation, a KRAS neoepitope comprising a KRAS G12V mutation, a KRAS neoepitope comprising a KRAS G12D mutation, and a KRAS neoepitope comprising a KRAS Q61H mutation. In some aspects, the KRAS neoepitope comprising the KRAS G12C mutation comprises the amino acid sequence KLVVVGACGV, VVVGACGVGK, or GACGVGKSAL, and combinations thereof. In some aspects, the KRAS neoepitope comprising the KRAS G12C mutation comprises the amino acid sequence VVVGACGVGK or KLVVVGACGV, and combinations thereof. In some aspects, the KRAS neoepitope comprising the KRAS G12V mutation comprises the amino acid sequence KLVVVGAVGV, VVVGAVGVGK, AVGVGKSAL, or GAVGVGKSAL, and combinations thereof. In some aspects, the KRAS neoepitope comprising the KRAS G12V mutation comprises the amino acid sequence VVGAVGVGK, VVVGAVGVGK, or AVGVGKSAL, and combinations thereof. In some aspects, the KRAS neoepitope comprising the KRAS G12D mutation comprises the amino acid sequence VVGADGVGK or VVVGADGVGK. In some aspects, the KRAS neoepitope comprising the KRAS Q61H mutation comprises the amino acid sequence ILDTAGHEEY. In some aspects, the KRAS-encoding nucleic acid sequence encodes each of the amino acid sequences VVVGACGVGK, VVVGADGVGK, VVGAVGVGK, and ILDTAGHEEY. In some aspects, the KRAS-encoding nucleic acid sequence encodes the amino acid sequence MTEYKLVVVGAVGVGKSALTIQLIQMTEYKLVVVGAVGVGKSALTIQLIQMTEYKLVVV GAVGVGKSALTIQLIQMTEYKLVVVGAVGVGKSALTIQLIQMTEYKLVVVGACGVGKSAL TIQLIQMTEYKLVVVGACGVGKSALTIQLIQMTEYKLVVVGACGVGKSALTIQLIQMTEYK LVVVGACGVGKSALTIQLIQ. In some aspects, the KRAS-encoding nucleic acid sequence encodes the amino acid sequence

MTEYKLVVVGACGVGKSALTIQLIQMTEYKLVVVGADGVGKSALT
IQLIQETCLLDILDTAGHEEYSAMRDQYMRMTEYKLVVVGADGVG
KSALTIQLIQMTEYKLVVVGAVGVGKSALTIQLIQMTEYKLVVVG
ACGVGKSALTIQLIQETCLLDILDTAGHEEYSAMRDQYMRMTEYK
LVVVGADGVGKSALTIQLIQMTEYKLVVVGAVGVGKSALTIQLIQ
MTEYKLVVVGACGVGKSALTIQLIQETCLLDILDTAGHEEYSAMR
DQYMRMTEYKLVVVGADGVGKSALTIQLIQMTEYKLVVVGAVGVG
KSALTIQLIQETCLLDILDTAGHEEYSAMRDQYMRMTEYKLVVVG
AVGVGKSALTIQLIQMTEYKLVVVGACGVGKSALTIQLIQ.

In some aspects, the two or more iterations comprises at least 3, at least 4, at least 5, at least 6, at least 7 iterations, or at least 8 iterations. In some aspects, the two or more iterations comprises at least 4 iterations. In some aspects, the two or more iterations comprises at least 2 iterations of the CTA-encoding nucleic acid sequence and the KRAS-encoding nucleic acid sequence. In some aspects, the two or more iterations comprises at least 2 iterations of the KRAS-encoding nucleic acid sequence and the KRAS-encoding nucleic acid sequence. In some aspects, the two or more iterations comprises at least 2 iterations of the CTA-encoding nucleic acid sequence and at least 2 iterations of the KRAS-encoding nucleic acid sequence. In some aspects, the two or more iterations comprises at least 2 iterations of the CTA-encoding nucleic acid sequence and at least 4 iterations of the KRAS-encoding nucleic acid sequence. In some aspects, the CTA-encoding nucleic acid sequence and the KRAS-encoding nucleic acid sequence are encoded in a single cassette, and wherein the single cassette encodes the amino acid sequence

ELGVMGVYDGREHTVYGEPRKELGVMGVYDGREHTVYGEPRKGID
VKEVDPTSHSYVLVTSGPRALAETSYVKVLEHVVRVNARVRGPRA
LAETSYVKVLEYVIKVSARVRFFFFLNMLGVYDGEEHSVFGEPWF
QRNTGEMSSNSTALALVRPSSSGLINSNTDNNLAVYDLSRDSPQG
ASALPTTISFTCWRQGIELMEVDPIGHLYIFATCFQRNTGEMSSN
STALALVRPSSSGLINSNTDNNLAVYDLSRDSPQGASALPTTISF
TCWRQGIELMEVDPIGHLYIFATCGPRALAETSYVKVLEHVVRVN
ARVRGPRALAETSYVKVLEYVIKVSARVRFFFFLNMLGVYDGEEH
SVFGEPWGIDVKEVDPTSHSYVLVTSGGSGGVRAEGRGSLLTCGD
VEENPGPMAGMTEYKLVVVGAVGVGKSALTIQLIMTEYKLVVVGA
VGVGKSALTIQLIMTEYKLVVVGAVGVGKSALTIQLIMTEYKLVV
VGAVGVGKSALTIQLIMTEYKLVVVGACGVGKSALTIQLIMTEYK
LVVVGACGVGKSALTIQLIMTEYKLVVVGACGVGKSALTIQLIMT
EYKLVVVGACGVGKSALTIQLIQ.

In some aspects, the antigen-encoding vaccine system comprises any one of the epitope-encoding nucleic acid sequences of Table 2B. In some aspects, the antigen-encoding vaccine system comprises a epitope-encoding nucleic acid sequence encoding any of the amino acid sequences of Table 2B.

In some aspects, the CTA-encoding nucleic acid sequence and the KRAS-encoding nucleic acid sequence are encoded in a single cassette, and wherein separate promoter nucleotide sequences provide for transcription of one or more of the separate open reading frames encoding the CTA-associated MHC class I epitope and the KRAS-associated MHC class I epitope, respectively. In some aspects, the separate promoter nucleotide sequences are different, optionally wherein the separate promoter nucleotide sequences are selected from the group consisting of a CMV, SV40, EF-1, RSV, PGK, HSA, MCK and a EBV promoter sequence, further optionally wherein the promoters comprise a TETr controlled promoter, further optionally wherein the TETr controlled promoter comprises a TETr controlled CMV-derived promoter or a TETr controlled EF-1-derived promoter. In some aspects, each of the separate promoters comprises a subgenomic promoter sequence, optionally wherein the subgenomic promoter sequence comprises a 26S subgenomic promoter sequence.

In some aspects, the CTA-encoding nucleic acid sequence and the KRAS-encoding nucleic acid sequence are encoded in a single cassette, and wherein the CTA-associated MHC class I epitope and the KRAS-associated MHC class I epitope are capable of being expressed as a single polypeptide. In some aspects, the CTA-associated MHC class I epitope and the KRAS-associated MHC class I epitope are linked by a 2A ribosome skipping sequence element.

In some aspects, each of the CTA-associated MHC class I epitope encoding nucleic acid sequences comprises at least two iterations. In some aspects, each of the KRAS-associated MHC class I epitope encoding nucleic acid sequences comprises at least two iterations. In some aspects, each of the CTA-associated MHC class I epitope encoding nucleic acid sequences and each of the KRAS-associated MHC class I epitope encoding nucleic acid sequences comprises at least two iterations.

In some aspects, the CTA-encoding nucleic acid sequence and/or the KRAS-encoding nucleic acid sequence, and optionally each CTA-encoding nucleic acid sequence and KRAS-encoding nucleic acid sequence, are described, from 5′ to 3′, by the formula (L5_{b}-N_{c}-L3_{d}), wherein N comprises a distinct epitope-encoding nucleic acid sequence that encodes the MHC epitope associated with each of the CTA-encoding nucleic acid sequences and the KRAS-encoding nucleic acid sequences, where c=1, L5 comprises a 5′ linker sequence, where b=0 or 1, and L3 comprises a 3′ linker sequence, where d=0 or 1.

In some aspects, each N encodes an epitope 7-15 amino acids in length, L5 is a native 5′ linker sequence that encodes a native N-terminal amino acid sequence of the epitope, and wherein the 5′ linker sequence encodes a peptide that is at least 2 amino acids in length, and optionally between 2-20 amino acids in length, and L3 is a native 3′ linker sequence that encodes a native C-terminal amino acid sequence of the epitope, and wherein the 3′ linker sequence encodes a peptide that is at least 2 amino acids in length, and optionally between 2-20 amino acids in length, and optionally wherein the CTA-encoding nucleic acid sequence and/or the KRAS-encoding nucleic acid sequence encodes a polypeptide that is between 12 and 35 amino acids in length.

In some aspects, one or both of the CTA-encoding nucleic acid sequence and the KRAS-encoding nucleic acid sequence, and optionally each of the CTA-encoding nucleic acid sequence and the KRAS-encoding nucleic acid sequences, encodes an epitope at least 7 amino acids in length. In some aspects, wherein one or both of the CTA-encoding nucleic acid sequence and the KRAS-encoding nucleic acid sequence, and optionally each of the CTA-encoding nucleic acid sequence and the KRAS-encoding nucleic acid sequences, encodes an epitope 7-15 amino acids in length. In some aspects, one or both of the CTA-encoding nucleic acid sequence and the KRAS-encoding nucleic acid sequence, and optionally each of the CTA-encoding nucleic acid sequence and the KRAS-encoding nucleic acid sequences, is a nucleotide sequence at least 21 nucleotides in length. In some aspects, one or both of the CTA-encoding nucleic acid sequence and the KRAS-encoding nucleic acid sequence, and optionally each of the CTA-encoding nucleic acid sequence and the KRAS-encoding nucleic acid sequences, is a nucleotide sequence 75 nucleotides in length.

In some aspects, the antigen-encoding vaccine system comprises one or more vectors, wherein each of the one or more vectors independently comprise: (a) a vector backbone comprising, wherein the backbone comprises: (i) a promoter nucleotide sequence; and (ii) a polyadenylation (poly(A)) sequence, and optionally wherein the vector backbone comprises an adenoviral vector or a self-amplifying viral vector, optionally wherein the adenoviral vector comprises a chimpanzee adenovirus vector, optionally wherein the chimpanzee adenovirus vector is a ChAdV68 vector, or optionally wherein the self-amplifying viral vector comprises an alphavirus vector, optionally wherein the alphavirus vector is a Venezuelan equine encephalitis virus vector; and (b) a cassette, wherein the cassette comprises: (i) the CTA-encoding nucleic acid sequence; or (ii) the KRAS-encoding nucleic acid sequence; or (iii) both the CTA-encoding nucleic acid sequence and the KRAS-encoding nucleic acid sequence.

In some aspects, the at least two iterations comprises a number of iterations sufficient to stimulate a greater immune response relative to an antigen-encoding nucleic acid sequence comprising a single iteration of the at least one epitope-encoding nucleic acid sequence. In some aspects, the at least two iterations comprises a number of iterations sufficient to stimulate an immune response, and a single iteration of the at least one epitope-encoding nucleic acid sequence is insufficient to stimulate the immune response or insufficient to stimulate a detectable immune response. In some aspects, the immune response is an expansion of epitope-specific T cells following in vivo immunization with the composition for delivery of the antigen expression system. In some aspects, the immune response is increased activation of epitope-specific T cells and/or increased epitope-specific killing by epitope-specific T cells following in vivo immunization with the composition for delivery of the antigen expression system.

In some aspects, the composition further comprises a nanoparticulate delivery vehicle, wherein the nanoparticulate delivery vehicle encapsulates the CTA-encoding nucleic acid sequence and/or the KRAS-encoding nucleic acid sequence. In some aspects, the CTA-encoding nucleic acid sequence and the KRAS-encoding nucleic acid sequence are formulated in the same nanoparticulate delivery vehicle. In some aspects, the CTA-encoding nucleic acid sequence and the KRAS-encoding nucleic acid sequence are formulated in separate nanoparticulate delivery vehicles, and wherein the composition comprises a mixture of the separate nanoparticulate delivery vehicles.

In some aspects, the vaccine system does not encode a non-therapeutic MHC class I or class II epitope nucleic acid sequence comprising a translated, wild-type nucleic acid sequence, wherein the non-therapeutic epitope is predicted to be displayed on an MHC allele of the subject. In some aspects, the non-therapeutic predicted MHC class I or class II epitope sequence is a junctional epitope sequence formed by adjacent sequences in the cassette.

In some aspects, the cassette is integrated between the at least one promoter nucleotide sequence and the at least one poly(A) sequence.

In some aspects, the one or more vectors comprise: (i) one or more +-stranded RNA vectors; (ii) a 5′ 7-methylguanosine (m7 g) cap; (iii) RNA vectors produced by in vitro transcription; and/or (iv) vectors that are self-replicating within a mammalian cell.

In some aspects, the backbone comprises at least one nucleotide sequence of a Venezuelan equine encephalitis virus. In some aspects, the vector backbone comprises at least sequences for nonstructural protein-mediated amplification, a 26S promoter sequence, and a poly(A) sequence encoded by the nucleotide sequence of the Venezuelan equine encephalitis virus, wherein sequences for nonstructural protein-mediated amplification are selected from the group consisting of: an alphavirus 5′ UTR, a 51-nt CSE, a 24-nt CSE, a 26S subgenomic promoter sequence, a 19-nt CSE, an alphavirus 3′ UTR, or combinations thereof, and/or wherein the backbone does not encode structural virion proteins capsid, E2 and E1. In some aspects, the Venezuelan equine encephalitis virus comprises the sequence of SEQ ID NO:3 or SEQ ID NO:5 further comprising a deletion between base pair 7544 and 11175, wherein the antigen cassette is inserted at position 7544 to replace the deletion between base pairs 7544 and 11175 as set forth in the sequence of SEQ ID NO:3 or SEQ ID NO:5.

In some aspects, the backbone comprises at least one nucleotide sequence of a chimpanzee adenovirus vector. In some aspects, the chimpanzee adenovirus vector is a ChAdV68 vector, optionally wherein the ChAdV68 vector comprises a ChAdV68 vector backbone comprising:
the sequence set forth in SEQ ID NO:1;
the sequence set forth in SEQ ID NO:1, except that the sequence is fully deleted or functionally deleted in at least one gene selected from the group consisting of the chimpanzee adenovirus E1A, E1B, E2A, E2B, E3, E4, L1, L2, L3, L4, and L5 genes of the sequence set forth in SEQ ID NO:1, optionally wherein the sequence is fully deleted or functionally deleted in: (1) E1A and E1B; (2) E1A, E1B, and E3; or (3) E1A, E1B, E3, and E4 of the sequence set forth in SEQ ID NO:1;
a gene or regulatory sequence obtained from the sequence of SEQ ID NO:1, optionally wherein the gene is selected from the group consisting of the chimpanzee adenovirus inverted terminal repeat (ITR), E1A, E1B, E2A, E2B, E3, E4, L1, L2, L3, L4, and L5 genes of the sequence set forth in SEQ ID NO:1;
a partially deleted E4 gene comprising a deleted or partially-deleted E4orf2 region and a deleted or partially-deleted E4orf3 region, and optionally a deleted or partially-deleted E4orf4 region;
at least nucleotides 2 to 36,518 of the sequence set forth in SEQ ID NO:1 and further comprising: (1) an E1 deletion of at least nucleotides 577 to 3403 of the sequence shown in SEQ ID NO:1, (2) an E3 deletion of at least nucleotides 27,125 to 31,825 of the sequence shown in SEQ ID NO:1, and (3) an E4 deletion of at least nucleotides 34,916 to 35,642 of the sequence shown in SEQ ID NO:1; optionally wherein the antigen cassette is inserted within the E1 deletion;
one or more deletions between base pair number 577 and 3403 or between base pair 456 and 3014, and optionally wherein the vector further comprises one or more deletions between base pair 27,125 and 31,825 or between base pair 27,816 and 31,333 of the sequence set forth in SEQ ID NO:1; or
one or more deletions between base pair number 3957 and 10346, base pair number 21787 and 23370, and base pair number 33486 and 36193 of the sequence set forth in SEQ ID NO:1, and
optionally wherein the cassette is inserted in the ChAdV vector backbone at the E1 region, E3 region, and/or any deleted AdV region that allows incorporation of the cassette.

In some aspects, the at least one promoter nucleotide sequence is the native subgenomic promoter nucleotide sequence encoded by the backbone, optionally a 26S promoter nucleotide sequence. In some aspects, the vector comprises multiple subgenomic promoter nucleotide sequence, wherein each subgenomic promoter nucleotide sequence are operably linked to and provide for transcription of one or more separate open reading frames in the cassette. In some aspects, the at least one promoter sequence is a regulatable promoter, optionally wherein the regulatable promoter is a tetracycline (TET) repressor protein (TETr) controlled promoter, optionally wherein the regulatable promoter comprises multiple TET operator (TETo) sequences 5′ or 3′ of a RNA polymerase binding sequence of the promoter.

In some aspects, each of the MHC class I epitopes is predicted or validated to be capable of presentation by at least one HLA allele, wherein each antigen/HLA pair has an antigen/HLA prevalence of at least 0.01% in a population. In some aspects, each of the MHC class I epitopes is predicted or validated to be capable of presentation by at least one HLA allele, wherein each antigen/HLA pair has an antigen/HLA prevalence of at least 0.1% in a population. In some aspects, the CTA-associated MHC class I epitope is validated to be capable of presentation by at least one HLA allele, wherein each antigen/HLA pair has an antigen/HLA prevalence of at least 0.1% in a population. In some aspects, the CTA-associated MHC class I epitope is validated to be capable of presentation by at least one HLA allele, wherein each antigen/HLA pair has an antigen/HLA prevalence of at least 0.5% in a population. In some aspects, each of the CTA-associated MHC class I epitope is validated to be capable of presentation by at least one HLA allele, wherein each antigen/HLA pair has an antigen/HLA prevalence of at least 0.1% in a population. In some aspects, each of the CTA-associated MHC class I epitope is validated to be capable of presentation by at least one HLA allele, wherein each antigen/HLA pair has an antigen/HLA prevalence of at least 0.5% in a population. In some aspects, the at least one HLA allele is HLA A*01:01, HLA A*02:01, B*44:02, B*44:05, B*40:01, B*40:02, B*41:02, B*35:01, B*15:01, A*33:03, A*02:05, A*11:01, C*03:04, A*29:02, C*15:02, and/or B*07:02.

Also disclosed herein is a pharmaceutical composition or compositions comprising any of the vaccine systems disclosed herein and a pharmaceutically acceptable carrier. In some aspects, CTA-encoding nucleic acid sequence and the RAS-encoding nucleic acid sequence are co-formulated. In some aspects, the CTA-encoding nucleic acid sequence and the KRAS-encoding nucleic acid sequence are formulated in separate pharmaceutical compositions.

Also disclosed herein is an isolated nucleotide sequence or set of isolated nucleotide sequences comprising the antigen-encoding vaccine system disclosed herein, and optionally one or more elements obtained from the sequence of SEQ ID NO:3 or SEQ ID NO:5, optionally wherein the one or more elements are selected from the group consisting of the sequences necessary for nonstructural protein-mediated amplification, the 26S promoter nucleotide sequence, the poly(A) sequence, and the nsP1-4 genes of the sequence set forth in SEQ ID NO:3 or SEQ ID NO:5, and optionally wherein the nucleotide sequence is cDNA.

Also disclosed herein is a vector or set of vectors comprising any one of the nucleotide sequences disclosed herein.

Also disclosed herein is an isolated cell comprising the nucleotide sequence or set of isolated nucleotide sequences or vectors disclosed herein, optionally wherein the cell is a BHK-21, CHO, HEK293 or variants thereof, 911, HeLa, A549, LP-293, PER.C6, or AE1-2a cell.

Also disclosed herein is a kit comprising any of the vaccine systems disclosed herein and instructions for use. In some aspects, the kit comprises the CTA-encoding nucleic acid sequence and the KRAS-encoding nucleic acid sequence co-formulated in a pharmaceutical composition. In some aspects, the kit comprises a first pharmaceutical composition comprising the CTA-encoding nucleic acid sequence and a second pharmaceutical composition comprising the KRAS-encoding nucleic acid sequence.

Also disclosed herein is a method for treating a subject with cancer, the method comprising administering to the subject any of the vaccine systems or pharmaceutical compositions disclosed herein. In some aspects, the cancer is non-small cell lung cancer (NSCLC).

Also disclosed herein is a method for stimulating an immune response in a subject, the method comprising administering to the subject any of the vaccine systems or pharmaceutical compositions disclosed herein.

In some aspects, the subject expresses at least one HLA allele predicted or known to present the CTA-associated MHC class I epitope, optionally wherein the at least one HLA allele is HLA A*01:01, HLA A*02:01, B*44:02, B*44:05, B*40:01, B*40:02, B*41:02, B*35:01, B*15:01, A*33:03, A*02:05, A*11:01, C*03:04, A*29:02, C*15:02, and/or B*07:02.

In some aspects, the method further comprises administering to the subject a second vaccine composition. In some aspects, the second vaccine composition is administered prior to the administration of the vaccine system or the pharmaceutical composition. In some aspects, the second vaccine composition is administered subsequent to the administration of the vaccine system or the pharmaceutical composition. In some aspects, the second vaccine composition is the same as the vaccine system or the pharmaceutical composition. In some aspects, the second vaccine composition is different from the vaccine system or the pharmaceutical composition.

Also disclosed herein is a method of manufacturing the one or more vectors of any of the vaccine systems disclosed herein, the method comprising: (a) obtaining a linearized DNA sequence comprising the backbone and the cassette; (b) in vitro transcribing the linearized DNA sequence by addition of the linearized DNA sequence to an in vitro transcription reaction containing all the necessary components to transcribe the linearized DNA sequence into RNA, optionally further comprising in vitro addition of the m7 g cap to the resulting RNA; and (c) isolating the one or more vectors from the in vitro transcription reaction.

Also disclosed herein is a method of manufacturing any of the vaccine systems disclosed herein for delivery of the antigen-encoding vaccine system, the method comprising: (a) providing components for the nanoparticulate delivery vehicle; (b) providing the antigen-encoding vaccine system; and (c) contacting the components for the nanoparticulate delivery vehicle and the antigen expression system under conditions sufficient for the nanoparticulate delivery vehicle and the antigen-encoding vaccine system to produce a delivery composition for delivery of the antigen-encoding vaccine system.

In some aspects, the conditions are provided by microfluidic mixing. In some aspects, the CTA-encoding nucleic acid sequence and the KRAS-encoding nucleic acid sequence are encoded in the same cassette and/or vector. In some aspects, the CTA-encoding nucleic acid sequence and the KRAS-encoding nucleic acid sequence are encoded on separate vectors and mixed prior to the contacting step (c). In some aspects, the CTA-encoding nucleic acid sequence and the KRAS-encoding nucleic acid sequence are encoded on separate vectors, and wherein the separate vectors are independently contacted with the components for the nanoparticulate delivery vehicle to produce a first delivery composition comprising the CTA-encoding nucleic acid sequence and a second delivery composition comprising the KRAS-encoding nucleic acid sequence. In some aspects, the first delivery composition and the second delivery composition are mixed subsequent to the contacting step (c).

Also disclosed herein is a method for treating a subject with a disease, optionally wherein the disease is cancer, the method comprising administering to the subject an antigen-encoding vaccine system, wherein the antigen-encoding vaccine system comprises: (a) a Cancer Testis Antigen (CTA)-encoding nucleic acid sequence encoding a CTA-associated MHC class I epitope; and (b) a KRAS-encoding nucleic acid sequence encoding a KRAS-associated MHC class I epitope.

Also disclosed herein is a method for treating a subject with a disease, optionally wherein the disease is cancer, the method comprising administering to the subject an antigen-encoding vaccine system, wherein the antigen-encoding vaccine system comprises: (i) a CTA-encoding nucleic acid sequence A (E_{A}); and (ii) a KRAS-encoding nucleic acid sequence B (E_{B}), wherein E_{A} and E_{B} each encode one MHC epitope, wherein E_{A} encodes a CTA-associated MHC class I epitope, and wherein E_{B} encodes a KRAS-associated MHC class I epitope.

In some aspects, the antigen-encoding vaccine system comprises any one of the vaccine systems disclosed here. In some aspects, the antigen-encoding vaccine system comprises any one of the pharmaceutical compositions disclosed herein.

In some aspects, the antigen-encoding vaccine system is administered as a priming dose. In some aspects, the antigen-encoding vaccine system is administered as one or more boosting doses. In some aspects, the boosting dose is different than the priming dose. In some aspects, (a) the priming dose comprises a chimpanzee adenovirus vector and the boosting dose comprises an alphavirus vector; or (b) the priming dose comprises an alphavirus vector and the boosting dose comprises a chimpanzee adenovirus vector.

In some aspects, the boosting dose is the same as the priming dose. In some aspects, the injection site of the one or more boosting doses is as close as possible to the injection site of the priming dose.

In some aspects, the method further comprises determining or having determined the HLA-haplotype of the subject, optionally wherein the HLA-haplotype determined of the subject comprises an HLA allele predicted or validated to present at least one of the CTA-associated MHC class I epitopes encoded by the antigen-encoding cassette, optionally wherein the HLA allele is HLA A*01:01, HLA A*02:01, B*44:02, B*44:05, B*40:01, B*40:02, B*41:02, B*35:01, B*15:01, A*33:03, A*02:05, A*11:01, C*03:04, A*29:02, C*15:02, and/or B*07:02.

In some aspects, the CTA-encoding nucleic acid sequence and the KRAS-encoding nucleic acid sequence are co-administered. In some aspects, the CTA-encoding nucleic acid sequence and the KRAS-encoding nucleic acid sequence are co-formulated in a single delivery composition. In some aspects, the CTA-encoding nucleic acid sequence and the KRAS-encoding nucleic acid sequence are formulated in a separate delivery compositions. In some aspects, the separate delivery compositions are administered at separate injection sites. In some aspects, the administration at separate injection sites comprises bilateral administration. In some aspects, the separate delivery compositions are mixed prior to co-administration.

Also disclosed herein is: an antigen-encoding cassette, or a polypeptide sequence encoded by the cassette, wherein the antigen-encoding cassette comprises at least one antigen-encoding nucleic acid sequence described, from 5′ to 3′, by the formula:

(Eₓ-(E^{N}ₙ)_{y})_{z}

wherein E represents a nucleotide sequence comprising a distinct epitope-encoding nucleic acid sequence, n represents the number of separate distinct epitope-encoding nucleic acid sequences and is any integer including 0, E^{N} represents a nucleotide sequence comprising the separate distinct epitope-encoding nucleic acid sequence for each corresponding n, for each iteration of z: x=0 or 1, y=0 or 1 for each n, and at least one of x or y=1, and z=1 or greater, optionally wherein the antigen-encoding nucleic acid sequence comprises at least two iterations of E, a given E^{N}, or a combination thereof, and at least one of the distinct epitope-encoding nucleic acid sequences encodes a distinct shared Cancer Testis Antigen (CTA)-associated MHC class I epitope, optionally wherein at least one of the distinct epitope-encoding nucleic acid sequences encoding the CTA comprises at least two iterations.

In some aspects, the CTA-associated MHC class I epitope is selected from the group consisting of: a MAGEA1 MHC class I epitope encoding nucleic acid sequence, a MAGEA3 MHC class I epitope encoding nucleic acid sequence, a MAGEA4 MHC class I epitope encoding nucleic acid sequence, a MAGEA6 MHC class I epitope encoding nucleic acid sequence, a MAGEA8 MHC class I epitope encoding nucleic acid sequence, a MAGEA11 MHC class I epitope encoding nucleic acid sequence, a MAGEB2 MHC class I epitope encoding nucleic acid sequence, a CTCFL MHC class I epitope encoding nucleic acid sequence, and a CT83 MHC class I epitope encoding nucleic acid sequence. In some aspects, the CTA-associated MHC class I epitope is selected from the group consisting of: FVQENYLEY, EVDPTSHSY, NTDNNLAVY, EVDPIGHLY, GVYDGREHTV, ALREEGEGV, KVLEYVIKV, GVYDGEEHSV, KLVELEHTL, AETSYVKVL, KVLEHVVRV, EADPTGHSY, SALPTTISF, GVYDGREHTVY, TQHFVQENY, EYVIKVSAR, LVRPSSSGL, and GEMSSNSTAL.

In some aspects, each of the distinct epitope-encoding nucleic acid sequences comprises at least two iterations of a distinct shared Cancer Testis Antigen (CTA)-associated MHC class I epitope.

In some aspects, the at least one distinct epitope-encoding nucleic acid sequences encoding the CTA-associated MHC class I epitope comprises at least 3, at least 4, at least 5, at least 6, at least 7 iterations, or at least 8 iterations. In some aspects, the at least one distinct epitope-encoding nucleic acid sequences encoding the CTA-associated MHC class I epitope comprises at least 3 iterations. In some aspects, the at least one distinct epitope-encoding nucleic acid sequences encoding the CTA-associated MHC class I epitope comprises at least 4 iterations. In some aspects, the antigen-encoding cassette comprises antigen-encoding nucleic acid sequences encoding: each of a MAGEA1 MHC class I epitope encoding nucleic acid sequence, a MAGEA4 MHC class I epitope encoding nucleic acid sequence, a MAGEB2 MHC class I epitope encoding nucleic acid sequence, a MAGEA11 MHC class I epitope encoding nucleic acid sequence, a MAGEA3 MHC class I epitope encoding nucleic acid sequence, and a CT83 MHC class I epitope encoding nucleic acid sequence, each of a MAGEA6 MHC class I epitope encoding nucleic acid sequence, a MAGEA11 MHC class I epitope encoding nucleic acid sequence, a MAGEA3 MHC class I epitope encoding nucleic acid sequence, and a CT83 MHC class I epitope encoding nucleic acid sequence, each of a MAGEA1 MHC class I epitope encoding nucleic acid sequence, a MAGEA4 MHC class I epitope encoding nucleic acid sequence, a MAGEB2 MHC class I epitope encoding nucleic acid sequence, a MAGEA11 MHC class I epitope encoding nucleic acid sequence, and a CT83 MHC class I epitope encoding nucleic acid sequence, or each of a MAGEA3 MHC class I epitope encoding nucleic acid sequence, a MAGEA4 MHC class I epitope encoding nucleic acid sequence, a MAGEB2 MHC class I epitope encoding nucleic acid sequence, a MAGEA11 MHC class I epitope encoding nucleic acid sequence, and a CT83 MHC class I epitope encoding nucleic acid sequence. In some aspects, each of the MHC class I epitope encoding nucleic acid sequence comprises at least two iterations.

In some aspects, each E or E^{N} independently comprises a nucleotide sequence described, from 5′ to 3′, by the formula (L5_{b}-N_{c}-L3_{d}), wherein N comprises the distinct epitope-encoding nucleic acid sequence associated with each E or E^{N}, where c=1, L5 comprises a 5′ linker sequence, where b=0 or 1, and L3 comprises a 3′ linker sequence, where d=0 or 1. In some aspects, each N encodes an epitope 7-15 amino acids in length, L5 is a native 5′ linker sequence that encodes a native N-terminal amino acid sequence of the epitope, and wherein the 5′ linker sequence encodes a peptide that is at least 2 amino acids in length, and L3 is a native 3′ linker sequence that encodes a native C-terminal amino acid sequence of the epitope, and wherein the 3′ linker sequence encodes a peptide that is at least 2 amino acids in length.

In some aspects, each of the epitope-encoding nucleic acid sequences encodes a polypeptide that is between 12 and 35 amino acids in length.

In some aspects, each E and E^{N} encodes an epitope at least 7 amino acids in length. In some aspects, each E and E^{N} encodes an epitope 7-15 amino acids in length. In some aspects, each E and E^{N} is a nucleotide sequence at least 21 nucleotides in length. In some aspects, each E and E^{N} is a nucleotide sequence 75 nucleotides in length.

In some aspects, at least one of the distinct epitope-encoding nucleic acid sequences encodes a distinct KRAS-associated MHC class I neoepitope. In some aspects, one or more of the distinct KRAS-associated MHC class I neoepitopes independently comprises a KRAS G12C mutation, a KRAS G12V mutation, a KRAS G12D mutation, or a KRAS Q61H mutation. In some aspects, one or more of the distinct KRAS-associated MHC class I neoepitopes independently comprises a KRAS G12C mutation or a KRAS G12V mutation. In some aspects, one or more of the distinct KRAS-associated MHC class I neoepitopes independently comprises each of a KRAS G12C mutation and a KRAS G12V mutation. In some aspects, the distinct KRAS-associated MHC class I neoepitope comprising the KRAS G12C mutation is selected from the group consisting of KLVVVGACGV, VVVGACGVGK, GACGVGKSAL, and combinations thereof. In some aspects, the distinct KRAS-associated MHC class I neoepitope comprising the KRAS G12V mutation is selected from the group consisting of KLVVVGAVGV, VVVGAVGVGK, AVGVGKSAL, GAVGVGKSAL, and combinations thereof. In some aspects, at least one of the distinct epitope-encoding nucleic acid sequences encoding the distinct KRAS-associated MHC class I neoepitope comprises at least two iterations of the distinct epitope-encoding nucleic acid sequences encoding the distinct KRAS-associated MHC class I neoepitope. In some aspects, each of the distinct epitope-encoding nucleic acid sequences encoding the distinct KRAS-associated MHC class I neoepitope comprises at least two iterations of the distinct epitope-encoding nucleic acid sequences encoding the distinct KRAS-associated MHC class I neoepitope.

Also disclosed herein is a composition for delivery of an antigen expression system, comprising: the antigen expression system, wherein the antigen expression system comprises one or more vectors, the one or more vectors comprising: (a) a vector backbone, wherein the backbone comprises: (i) at least one promoter nucleotide sequence, and (ii) optionally, at least one polyadenylation (poly(A)) sequence; and (b) a cassette, wherein the cassette comprises: (i) at least one antigen-encoding nucleic acid sequence, comprising: (I) at least one epitope-encoding nucleic acid sequence encodes a CTA-associated MHC class I epitope, optionally wherein at least one of the epitope-encoding nucleic acid sequences encoding the CTA comprises at least two iterations, and wherein each of the epitope-encoding nucleic acid sequences comprises; (A) optionally, a 5′ linker sequence, and (B) optionally, a 3′ linker sequence; (ii) optionally, a second promoter nucleotide sequence operably linked to the antigen-encoding nucleic acid sequence; and (iii) optionally, at least one MHC class II epitope-encoding nucleic acid sequence; (iv) optionally, at least one nucleic acid sequence encoding a GPGPG amino acid linker sequence (SEQ ID NO:56); and (v) optionally, at least one second poly(A) sequence, wherein the second poly(A) sequence is a native poly(A) sequence or an exogenous poly(A) sequence to the vector backbone, wherein if the second promoter nucleotide sequence is absent, the antigen-encoding nucleic acid sequence is operably linked to the at least one promoter nucleotide sequence, and wherein the at least one antigen-encoding nucleic acid sequence comprises at least two iterations of the epitope-encoding nucleic acid sequence encoding the CTA-associated MHC class I epitope.

Also disclosed herein is a composition for delivery of an antigen expression system, comprising: the antigen expression system, wherein the antigen expression system comprises one or more vectors, the one or more vectors comprising: (a) a vector backbone, wherein the backbone comprises: (i) at least one promoter nucleotide sequence, and (ii) at least one polyadenylation (poly(A)) sequence; and (b) a cassette, wherein the cassette comprises: (i) at least one antigen-encoding nucleic acid sequence, comprising: (I) at least 4 distinct epitope-encoding nucleic acid sequences linearly linked to each other, wherein each of the distinct epitope-encoding nucleic acid sequences encodes a CTA-associated MHC class I epitope, and wherein each of the epitope-encoding nucleic acid sequences comprises; (A) optionally, a 5′ linker sequence, and (B) optionally, a 3′ linker sequence; (ii) optionally, a second promoter nucleotide sequence operably linked to the antigen-encoding nucleic acid sequence; (iii) optionally, at least one MHC class II epitope-encoding nucleic acid sequence; (iv) optionally, at least one nucleic acid sequence encoding a GPGPG amino acid linker sequence (SEQ ID NO:56); and (v) optionally, at least one second poly(A) sequence, wherein the second poly(A) sequence is a native poly(A) sequence or an exogenous poly(A) sequence to the vector backbone, wherein if the second promoter nucleotide sequence is absent, the antigen-encoding nucleic acid sequence is operably linked to the at least one promoter nucleotide sequence, and wherein the at least one antigen-encoding nucleic acid sequence comprises at least two iterations of the each of the epitope-encoding nucleic acid sequences encoding the CTA-associated MHC class I epitope.

In some aspects, each of the CTA-associated MHC class I epitope is selected from the group consisting of: a MAGEA1 MHC class I epitope encoding nucleic acid sequence, a MAGEA3 MHC class I epitope encoding nucleic acid sequence, a MAGEA4 MHC class I epitope encoding nucleic acid sequence, a MAGEA6 MHC class I epitope encoding nucleic acid sequence, a MAGEA8 MHC class I epitope encoding nucleic acid sequence, a MAGEA11 MHC class I epitope encoding nucleic acid sequence, a MAGEB2 MHC class I epitope encoding nucleic acid sequence, a CTCFL MHC class I epitope encoding nucleic acid sequence, and a CT83 MHC class I epitope encoding nucleic acid sequence, and optionally, wherein each of the CTA-associated MHC class I epitope is selected from the group consisting of: FVQENYLEY, EVDPTSHSY, NTDNNLAVY, EVDPIGHLY, GVYDGREHTV, ALREEGEGV, KVLEYVIKV, GVYDGEEHSV, KLVELEHTL, AETSYVKVL, KVLEHVVRV, EADPTGHSY, SALPTTISF, GVYDGREHTVY, TQHFVQENY, EYVIKVSAR, LVRPSSSGL, GEMSSNSTAL, TVYGEPRKL, ALAETSYVK, TSYVKVLEH, YPSLREAAL, ALLEEEEGV, GPRQSLQQC, IAYPSLREAAL, and MEVDPIGHL.

Also disclosed herein is a composition for delivery of an antigen expression system, comprising: the antigen expression system, wherein the antigen expression system comprises one or more vectors, the one or more vectors comprising: (a) a vector backbone, wherein the vector backbone comprises a chimpanzee adenovirus vector, optionally wherein the chimpanzee adenovirus vector is a ChAdV68 vector, or an alphavirus vector, optionally wherein the alphavirus vector is a Venezuelan equine encephalitis virus vector; and (b) a cassette, optionally wherein the cassette is integrated between a native promoter nucleotide sequence native to the vector backbone and a poly(A) sequence, optionally wherein the poly(A) sequence is native to the vector backbone, wherein the cassette comprises: (i) at least one antigen-encoding nucleic acid sequence, comprising: (I) at least one epitope-encoding nucleic acid sequence encoding a CTA-associated MHC class I epitope, optionally comprising at least two distinct epitope-encoding nucleic acid sequences linearly linked to each other, each epitope-encoding nucleic acid sequence optionally comprising: (A) a MHC class I epitope encoding nucleic acid sequence, wherein the MHC class I epitope encoding nucleic acid sequence encodes a MHC class I epitope 7-15 amino acids in length, (B) a 5′ linker sequence, wherein the 5′ linker sequence encodes a native N-terminal amino acid sequence of the MHC class I epitope, and wherein the 5′ linker sequence encodes a peptide that is at least 3 amino acids in length, (C) a 3′ linker sequence, wherein the 3′ linker sequence encodes a native C-terminal acid sequence of the MHC class I epitope, and wherein the 3′ linker sequence encodes a peptide that is at least 3 amino acids in length, and wherein the cassette is operably linked to the native promoter nucleotide sequence, wherein each of the epitope-encoding nucleic acid sequences encodes a polypeptide that is between 12 and 35 amino acids in length, and wherein each 3′ end of each epitope-encoding nucleic acid sequence is linked to the 5′ end of the following epitope-encoding nucleic acid sequence with the exception of the final epitope-encoding nucleic acid sequence in the cassette; and (ii) at least two MHC class II epitope-encoding nucleic acid sequences comprising: (I) a PADRE MHC class II sequence (SEQ ID NO:48), (II) a Tetanus toxoid MHC class II sequence (SEQ ID NO:46), (III) a first nucleic acid sequence encoding a GPGPG amino acid linker sequence linking the PADRE MHC class II sequence and the Tetanus toxoid MHC class II sequence, (IV) a second nucleic acid sequence encoding a GPGPG amino acid linker sequence linking the 5′ end of the at least two MHC class II epitope-encoding nucleic acid sequences to the epitope-encoding nucleic acid sequences, (V) optionally, a third nucleic acid sequence encoding a GPGPG amino acid linker sequence at the 3′ end of the at least two MHC class II epitope-encoding nucleic acid sequences; (iii) optionally, a second promoter nucleotide sequence operably linked to the antigen-encoding nucleic acid sequence; and wherein if the second promoter nucleotide sequence is absent, the antigen-encoding nucleic acid sequence is operably linked to the native promoter nucleotide sequence, and wherein the at least one antigen-encoding nucleic acid sequence comprises at least two iterations of the epitope-encoding nucleic acid sequence encoding the CTA-associated MHC class I epitope.

In some aspects, an ordered sequence of each element of the cassette is described in the formula, from 5′ to 3′, comprising:

Pₐ-(L5_{b}-N_{c}-L3_{d})_{X}-(G5ₑ-U_{f})_{Y}-G3_{g}

wherein, P comprises the second promoter nucleotide sequence, where a=0 or 1, N comprises one of the distinct epitope-encoding nucleic acid sequences, where c=1, L5 comprises the 5′ linker sequence, where b=0 or 1, L3 comprises the 3′ linker sequence, where d=0 or 1, G5 comprises one of the at least one nucleic acid sequences encoding a GPGPG amino acid linker, where e=0 or 1, G3 comprises one of the at least one nucleic acid sequences encoding a GPGPG amino acid linker, where g=0 or 1, U comprises one of the at least one MHC class II epitope-encoding nucleic acid sequence, where f=1, X=1 to 400, where for each X the corresponding N_{c} is an epitope-encoding nucleic acid sequence, and Y=0, 1, or 2, where for each Y the corresponding U_{f} is an MHC class II epitope-encoding nucleic acid sequence.

In some aspects, for each X the corresponding N_{c} is the epitope-encoding nucleic acid sequence encoding the CTA-associated MHC class I epitope. In some aspects, for each Y the corresponding U_{f} is a distinct MHC class II epitope-encoding nucleic acid sequence. In some aspects, a=0, b=1, d=1, e=1, g=1, h=1, X=10, Y=2, the at least one promoter nucleotide sequence is a single native promoter nucleotide sequence native to the vector backbone, the at least one polyadenylation poly(A) sequence is a poly(A) sequence of at least 100 consecutive A nucleotides provided by the vector backbone, each N encodes an epitope 7-15 amino acids in length, L5 is a native 5′ linker sequence that encodes a native N-terminal amino acid sequence of the epitope, and wherein the 5′ linker sequence encodes a peptide that is at least 3 amino acids in length, L3 is a native 3′ linker sequence that encodes a native C-terminal amino acid sequence of the epitope, and wherein the 3′ linker sequence encodes a peptide that is at least 3 amino acids in length, U is each of a PADRE class II sequence and a Tetanus toxoid MHC class II sequence, the vector backbone comprises a chimpanzee adenovirus vector, optionally wherein the chimpanzee adenovirus vector is a ChAdV68 vector, or an alphavirus vector, optionally wherein the alphavirus vector is a Venezuelan equine encephalitis virus vector, optionally wherein the native promoter nucleotide sequence is a 26S promoter when the vector backbone comprises an alphavirus vector, and each of the MHC class I epitope-encoding nucleic acid sequences encodes a polypeptide that is between 12 and 35 amino acids in length.

In some aspects, the at least two iterations is at least 3, at least 4, at least 5, at least 6, at least 7, or at least 8 iterations. In some aspects, the at least two iterations is at least 8 iterations. In some aspects, the at least two iterations is between 2-3, between 2-4, between 2-5, between 2-6, between 2-7, or between 2-8 iterations. In some aspects, the at least two iterations is 7 iterations or less, 6 iterations or less, 5 iterations or less, 4 iterations or less, or 3 iterations or less. In some aspects, the at least one antigen-encoding nucleic acid sequence comprises at least two iterations of at least two distinct epitope-encoding nucleic acid sequences. In some aspects, the at least one antigen-encoding nucleic acid sequence comprises at least two iterations of at least 3, at least 4, at least 5, at least 6, at least 7, at least 8, at least 9, or at least 10 distinct epitope-encoding nucleic acid sequences. In some aspects, the at least two iterations are separated by at least one separate distinct epitope-encoding nucleic acid sequence. In some aspects, the at least two iterations are separated by at least 2 separate distinct epitope-encoding nucleic acid sequences. In some aspects, the at least two iterations, inclusive of the optional 5′ linker sequence and/or the optional 3′ linker sequence, are separated by at least 75 nucleotides. In some aspects, the at least two iterations, inclusive of the optional 5′ linker sequence and/or the optional 3′ linker sequence, are separated by at least 150 nucleotides, at least 300 nucleotides, or at least 675 nucleotides.

In some aspects, the at least two iterations, inclusive of the optional 5′ linker sequence and/or the optional 3′ linker sequence, are separated by at least 50 nucleotides, at least 100 nucleotides, at least 200 nucleotides, at least 250 nucleotides, at least 350 nucleotides, at least 400 nucleotides, at least 450 nucleotides, at least 500 nucleotides, at least 700 nucleotides, at least 700 nucleotides, at least 750 nucleotides, at least 800 nucleotides, at least 900 nucleotides, or at least 1000 nucleotides. In some aspects, the at least two iterations, inclusive of the optional 5′ linker sequence and/or the optional 3′ linker sequence, are separated by at least 10 nucleotides, at least 15 nucleotides, at least 20 nucleotides, at least 25 nucleotides, at least 30 nucleotides, at least 35 nucleotides, at least 40 nucleotides, at least 45 nucleotides, at least 50 nucleotides, at least 55 nucleotides, at least 60 nucleotides, at least 65 nucleotides, or at least 70 nucleotides.

In some aspects, the at least one antigen-encoding nucleic acid sequence is described, from 5′ to 3′, by the formula:

(Eₓ-(E^{N}ₙ)_{y})_{z}

wherein E represents a nucleotide sequence comprising a distinct epitope-encoding nucleic acid sequence, n represents the number of separate distinct epitope-encoding nucleic acid sequences and is any integer including 0, E^{N} represents a nucleotide sequence comprising the separate distinct epitope-encoding nucleic acid sequence for each corresponding n, for each iteration of z: x=0 or 1, y=0 or 1 for each n, and at least one of x or y=1, and z=1 or greater, optionally wherein the antigen-encoding nucleic acid sequence comprises at least two iterations of E, a given E^{N}, or a combination thereof, and at least one of the distinct epitope-encoding nucleic acid sequences encodes a distinct shared Cancer Testis Antigen (CTA)-associated MHC class I epitope, optionally wherein at least one of the distinct epitope-encoding nucleic acid sequences encoding the CTA comprises at least two iterations.

In some aspects, the CTA-associated MHC class I epitope is selected from the group consisting of: a MAGEA1 MHC class I epitope encoding nucleic acid sequence, a MAGEA3 MHC class I epitope encoding nucleic acid sequence, a MAGEA4 MHC class I epitope encoding nucleic acid sequence, a MAGEA6 MHC class I epitope encoding nucleic acid sequence, a MAGEA8 MHC class I epitope encoding nucleic acid sequence, a MAGEA11 MHC class I epitope encoding nucleic acid sequence, a MAGEB2 MHC class I epitope encoding nucleic acid sequence, a CTCFL MHC class I epitope encoding nucleic acid sequence, and a CT83 MHC class I epitope encoding nucleic acid sequence. In some aspects, the CTA-associated MHC class I epitope is selected from the group consisting of: FVQENYLEY, EVDPTSHSY, NTDNNLAVY, EVDPIGHLY, GVYDGREHTV, ALREEGEGV, KVLEYVIKV, GVYDGEEHSV, KLVELEHTL, AETSYVKVL, KVLEHVVRV, EADPTGHSY, SALPTTISF, GVYDGREHTVY, TQHFVQENY, EYVIKVSAR, LVRPSSSGL, GEMSSNSTAL, TVYGEPRKL, ALAETSYVK, TSYVKVLEH, YPSLREAAL, ALLEEEEGV, GPRQSLQQC, IAYPSLREAAL, and MEVDPIGHL.

In some aspects, the antigen-encoding cassette comprises antigen-encoding nucleic acid sequences encoding: each of a MAGEA1 MHC class I epitope encoding nucleic acid sequence, a MAGEA4 MHC class I epitope encoding nucleic acid sequence, a MAGEB2 MHC class I epitope encoding nucleic acid sequence, a MAGEA11 MHC class I epitope encoding nucleic acid sequence, a MAGEA3 MHC class I epitope encoding nucleic acid sequence, and a CT83 MHC class I epitope encoding nucleic acid sequence, each of a MAGEA6 MHC class I epitope encoding nucleic acid sequence, a MAGEA11 MHC class I epitope encoding nucleic acid sequence, a MAGEA3 MHC class I epitope encoding nucleic acid sequence, and a CT83 MHC class I epitope encoding nucleic acid sequence, each of a MAGEA1 MHC class I epitope encoding nucleic acid sequence, a MAGEA4 MHC class I epitope encoding nucleic acid sequence, a MAGEB2 MHC class I epitope encoding nucleic acid sequence, a MAGEA11 MHC class I epitope encoding nucleic acid sequence, and a CT83 MHC class I epitope encoding nucleic acid sequence, or each of a MAGEA3 MHC class I epitope encoding nucleic acid sequence, a MAGEA4 MHC class I epitope encoding nucleic acid sequence, a MAGEB2 MHC class I epitope encoding nucleic acid sequence, a MAGEA11 MHC class I epitope encoding nucleic acid sequence, and a CT83 MHC class I epitope encoding nucleic acid sequence.

In some aspects, one or more of the nucleic acid sequences encoding the CTA-associated MHC class I epitope comprises at least 2, at least 3, at least 4, at least 5, at least 6, or at least 7 iterations. In some aspects, each of the nucleic acid sequences encoding the CTA-associated MHC class I epitope comprises at least 2, at least 3, at least 4, at least 5, at least 6, or at least 7 iterations. In some aspects, one or more of the nucleic acid sequences encoding the CTA-associated MHC class I epitope comprises at least 4 iterations. In some aspects, each of the nucleic acid sequences encoding the CTA-associated MHC class I epitope comprises at least 4 iterations.

In some aspects, each of the CTA-associated MHC class I epitopes are selected from the group consisting of: FVQENYLEY, EVDPTSHSY, NTDNNLAVY, EVDPIGHLY, GVYDGREHTV, ALREEGEGV, KVLEYVIKV, GVYDGEEHSV, KLVELEHTL, AETSYVKVL, KVLEHVVRV, EADPTGHSY, SALPTTISF, GVYDGREHTVY, TQHFVQENY, EYVIKVSAR, LVRPSSSGL, GEMSSNSTAL, TVYGEPRKL, ALAETSYVK, TSYVKVLEH, YPSLREAAL, ALLEEEEGV, GPRQSLQQC, IAYPSLREAAL, and MEVDPIGHL.

In some aspects, the at least two iterations comprises a number of iterations, or z comprises a number, sufficient to stimulate a greater immune response relative to an antigen-encoding nucleic acid sequence comprising a single iteration of the at least one epitope-encoding nucleic acid sequence. In some aspects, the at least two iterations comprises a number of iterations, or z comprises a number, sufficient to stimulate an immune response, and a single iteration of the at least one epitope-encoding nucleic acid sequence is insufficient to stimulate the immune response or insufficient to stimulate a detectable immune response. In some aspects, the immune response is an expansion of epitope-specific T cells following in vivo immunization with the composition for delivery of the antigen expression system. In some aspects, the immune response is increased activation of epitope-specific T cells and/or increased epitope-specific killing by epitope-specific T cells following in vivo immunization with the composition for delivery of the antigen expression system.

In some aspects, the composition further comprises a nanoparticulate delivery vehicle. In some aspects, the nanoparticulate delivery vehicle is a lipid nanoparticle (LNP). In some aspects, the LNP comprises ionizable amino lipids. In some aspects, the ionizable amino lipids comprise MC3-like (dilinoleylmethyl-4-dimethylaminobutyrate) molecules. In some aspects, the nanoparticulate delivery vehicle encapsulates the antigen expression system.

In some aspects, the nanoparticulate delivery vehicle encapsulates the antigen expression system. In some aspects, the cassette is integrated between the at least one promoter nucleotide sequence and the at least one poly(A) sequence. In some aspects, the second promoter is absent and the at least one promoter nucleotide sequence is operably linked to the antigen-encoding nucleic acid sequence. In some aspects, the one or more vectors comprise one or more +-stranded RNA vectors. In some aspects, the one or more +-stranded RNA vectors comprise a 5′ 7-methylguanosine (m7 g) cap. In some aspects, the one or more +-stranded RNA vectors are produced by in vitro transcription. In some aspects, the one or more vectors are self-replicating within a mammalian cell. In some aspects, the backbone comprises at least one nucleotide sequence of an Aura virus, a Fort Morgan virus, a Venezuelan equine encephalitis virus, a Ross River virus, a Semliki Forest virus, a Sindbis virus, or a Mayaro virus. In some aspects, the backbone comprises at least one nucleotide sequence of a Venezuelan equine encephalitis virus. In some aspects, the backbone comprises at least sequences for nonstructural protein-mediated amplification, a 26S promoter sequence, a poly(A) sequence, a nonstructural protein 1 (nsP1) gene, a nsP2 gene, a nsP3 gene, and a nsP4 gene encoded by the nucleotide sequence of the Aura virus, the Fort Morgan virus, the Venezuelan equine encephalitis virus, the Ross River virus, the Semliki Forest virus, the Sindbis virus, or the Mayaro virus. In some aspects, the backbone comprises at least sequences for nonstructural protein-mediated amplification, a 26S promoter sequence, and a poly(A) sequence encoded by the nucleotide sequence of the Aura virus, the Fort Morgan virus, the Venezuelan equine encephalitis virus, the Ross River virus, the Semliki Forest virus, the Sindbis virus, or the Mayaro virus. In some aspects, sequences for nonstructural protein-mediated amplification are selected from the group consisting of: an alphavirus 5′ UTR, a 51-nt CSE, a 24-nt CSE, a 26S subgenomic promoter sequence, a 19-nt CSE, an alphavirus 3′ UTR, or combinations thereof. In some aspects, the backbone does not encode structural virion proteins capsid, E2 and E1. In some aspects, the cassette is inserted in place of structural virion proteins within the nucleotide sequence of the Aura virus, the Fort Morgan virus, the Venezuelan equine encephalitis virus, the Ross River virus, the Semliki Forest virus, the Sindbis virus, or the Mayaro virus.

In some aspects, the Venezuelan equine encephalitis virus comprises the sequence of SEQ ID NO:3 or SEQ ID NO:5. In some aspects, the Venezuelan equine encephalitis virus comprises the sequence of SEQ ID NO:3 or SEQ ID NO:5 further comprising a deletion between base pair 7544 and 11175. In some aspects, the backbone comprises the sequence set forth in SEQ ID NO:6 or SEQ ID NO:7. In some aspects, the cassette is inserted at position 7544 to replace the deletion between base pairs 7544 and 11175 as set forth in the sequence of SEQ ID NO:3 or SEQ ID NO:5.

In some aspects, the insertion of the cassette provides for transcription of a polycistronic RNA comprising the nsP1-4 genes and the at least one antigen-encoding nucleic acid sequence, wherein the nsP1-4 genes and the at least one antigen-encoding nucleic acid sequence are in separate open reading frames.

In some aspects, the chimpanzee adenovirus vector is a ChAdV68 vector, optionally wherein the ChAdV68 vector comprises a ChAdV68 vector backbone comprising: the sequence set forth in SEQ ID NO:1; the sequence set forth in SEQ ID NO:1, except that the sequence is fully deleted or functionally deleted in at least one gene selected from the group consisting of the chimpanzee adenovirus E1A, E1B, E2A, E2B, E3, E4, L1, L2, L3, L4, and L5 genes of the sequence set forth in SEQ ID NO:1, optionally wherein the sequence is fully deleted or functionally deleted in: (1) E1A and E1B; (2) E1A, E1B, and E3; or (3) E1A, E1B, E3, and E4 of the sequence set forth in SEQ ID NO:1; a gene or regulatory sequence obtained from the sequence of SEQ ID NO:1, optionally wherein the gene is selected from the group consisting of the chimpanzee adenovirus inverted terminal repeat (ITR), E1A, E1B, E2A, E2B, E3, E4, L1, L2, L3, L4, and L5 genes of the sequence set forth in SEQ ID NO:1; a partially deleted E4 gene comprising a deleted or partially-deleted E4orf2 region and a deleted or partially-deleted E4orf3 region, and optionally a deleted or partially-deleted E4orf4 region; at least nucleotides 2 to 36,518 of the sequence set forth in SEQ ID NO:1 and further comprising: (1) an E1 deletion of at least nucleotides 577 to 3403 of the sequence shown in SEQ ID NO:1, (2) an E3 deletion of at least nucleotides 27,125 to 31,825 of the sequence shown in SEQ ID NO:1, and (3) an E4 deletion of at least nucleotides 34,916 to 35,642 of the sequence shown in SEQ ID NO:1; optionally wherein the antigen cassette is inserted within the E1 deletion; one or more deletions between base pair number 577 and 3403 or between base pair 456 and 3014, and optionally wherein the vector further comprises one or more deletions between base pair 27,125 and 31,825 or between base pair 27,816 and 31,333 of the sequence set forth in SEQ ID NO:1; or one or more deletions between base pair number 3957 and 10346, base pair number 21787 and 23370, and base pair number 33486 and 36193 of the sequence set forth in SEQ ID NO:1, and optionally wherein the cassette is inserted in the ChAdV vector backbone at the E1 region, E3 region, and/or any deleted AdV region that allows incorporation of the cassette.

In some aspects, the at least one promoter nucleotide sequence is the native 26S promoter nucleotide sequence encoded by the backbone. In some aspects, the at least one promoter nucleotide sequence is an exogenous RNA promoter. In some aspects, the second promoter nucleotide sequence is a 26S promoter nucleotide sequence. In some aspects, the second promoter nucleotide sequence comprises multiple 26S promoter nucleotide sequences, wherein each 26S promoter nucleotide sequence provides for transcription of one or more of the separate open reading frames.

In some aspects, one or more of the cassettes are at least 100, 200, 300, 400, 500, 600, 700, 800, or 900 nucleotides in length. In some aspects, one or more of the cassettes are at least 1000, 2000, 3000, 4000, 5000, 6000, 7000, 8000, 9000, or 10000 nucleotides in length. In some aspects, one or more of the cassettes is at least 3500 nucleotides in length. In some aspects, one or more of the cassettes is at least 6000 nucleotides in length.

In some aspects, at least one of the at least one antigen-encoding nucleic acid sequences encodes a polypeptide sequence or portion thereof that is presented by MHC class I on a cell surface, optionally a tumor cell surface.

In some aspects, each epitope-encoding nucleic acid sequence is linked directly to one another. In some aspects, at least one of the at least one epitope-encoding nucleic acid sequences is linked to a distinct epitope-encoding nucleic acid sequence with a nucleic acid sequence encoding a linker. In some aspects, the linker links two MHC class I sequences or an MHC class I sequence to an MHC class II sequence. In some aspects, the linker is selected from the group consisting of: (1) consecutive glycine residues, at least 2, 3, 4, 5, 6, 7, 8, 9, or 10 residues in length; (2) consecutive alanine residues, at least 2, 3, 4, 5, 6, 7, 8, 9, or 10 residues in length; (3) two arginine residues (RR); (4) alanine, alanine, tyrosine (AAY); (5) a consensus sequence at least 2, 3, 4, 5, 6, 7, 8, 9, or 10 amino acid residues in length that is processed efficiently by a mammalian proteasome; and (6) one or more native sequences flanking the antigen derived from the cognate protein of origin and that is at least 2, 3, 4, 5, 6, 7, 8, 9, 10, 11, 12, 13, 14, 15, 16, 17, 18, 19, 20, or 2-20 amino acid residues in length. In some aspects, the linker links two MHC class II sequences or an MHC class II sequence to an MHC class I sequence. In some aspects, the linker comprises the sequence GPGPG. In some aspects, at least one sequence of the at least one epitope-encoding nucleic acid sequences is linked, operably or directly, to a separate or contiguous sequence that enhances the expression, stability, cell trafficking, processing and presentation, and/or immunogenicity of the at least one epitope-encoding nucleic acid sequences of epitope encoded therefrom. In some aspects, the separate or contiguous sequence comprises at least one of: a ubiquitin sequence, a ubiquitin sequence modified to increase proteasome targeting (e.g., the ubiquitin sequence contains a Gly to Ala substitution at position 76), an immunoglobulin signal sequence (e.g., IgK), a major histocompatibility class I sequence, lysosomal-associated membrane protein (LAMP)-1, human dendritic cell lysosomal-associated membrane protein, and a major histocompatibility class II sequence; optionally wherein the ubiquitin sequence modified to increase proteasome targeting is A76.

In some aspects, at least one of the at least one epitope-encoding nucleic acid sequences encodes a polypeptide sequence or portion thereof that has increased binding affinity to its corresponding MHC allele relative to the translated, corresponding wild-type nucleic acid sequence. In some aspects, at least one of the at least one epitope-encoding nucleic acid sequences encodes a polypeptide sequence or portion thereof that has increased binding stability to its corresponding MHC allele relative to the translated, corresponding wild-type nucleic acid sequence. In some aspects, at least one of the at least one epitope-encoding nucleic acid sequences encodes a polypeptide sequence or portion thereof that has an increased likelihood of presentation on its corresponding MHC allele relative to the translated, corresponding wild-type nucleic acid sequence. In some aspects, the at least one alteration comprises a point mutation, a frameshift mutation, a non-frameshift mutation, a deletion mutation, an insertion mutation, a splice variant, a genomic rearrangement, or a proteasome-generated spliced antigen.

In some aspects, the tumor is selected from the group consisting of: lung cancer, melanoma, breast cancer, ovarian cancer, prostate cancer, kidney cancer, gastric cancer, colon cancer, testicular cancer, head and neck cancer, pancreatic cancer, bladder cancer, brain cancer, B-cell lymphoma, acute myelogenous leukemia, adult acute lymphoblastic leukemia, chronic myelogenous leukemia, chronic lymphocytic leukemia, T cell lymphocytic leukemia, non-small cell lung cancer, and small cell lung cancer. In some aspects, the tumor is a lung adenocarcinoma, ovarian serous cystadenocarcinoma, lung squamous cell carcinoma, colon cancer, or head and neck squamous cell carcinoma.

In some aspects, the at least one antigen-encoding nucleic acid sequence comprises at least 2-10, 2, 3, 4, 5, 6, 7, 8, 9, or 10 epitope-encoding nucleic acid sequences. In some aspects, the at least one antigen-encoding nucleic acid sequence comprises at least 11-20, 15-20, 11-100, 11-200, 11-300, 11-400, 11, 12, 13, 14, 15, 16, 17, 18, 19, 20 or up to 400 epitope-encoding nucleic acid sequences. In some aspects, the at least one antigen-encoding nucleic acid sequence comprises at least 2-400 epitope-encoding nucleic acid sequences and wherein at least two of the epitope-encoding nucleic acid sequences encode polypeptide sequences or portions thereof that are presented by MHC class I on a cell surface, optionally a tumor cell surface. In some aspects, the at least one antigen-encoding nucleic acid sequence comprises at least 2-10, 2, 3, 4, 5, 6, 7, 8, 9, or 10 antigen-encoding nucleic acid sequences. In some aspects, the at least one antigen-encoding nucleic acid sequence comprises at least 11-20, 15-20, 11-100, 11-200, 11-300, 11-400, 11, 12, 13, 14, 15, 16, 17, 18, 19, 20 or up to 400 antigen-encoding nucleic acid sequences. In some aspects, the at least one antigen-encoding nucleic acid sequence comprises at least 2-400 antigen-encoding nucleic acid sequences and wherein at least two of the antigen-encoding nucleic acid sequences encode polypeptide sequences or portions thereof that are presented by MHC class I on a cell surface, optionally a tumor cell surface. In some aspects, at least two of the epitope-encoding nucleic acid sequences encode polypeptide sequences or portions thereof that are presented by MHC class I on a cell surface, optionally a tumor cell surface.

In some aspects, when administered to the subject and translated, the CTA-associated MHC class I epitope is presented on antigen presenting cells resulting in an immune response targeting CTA-associated MHC class I epitope on the tumor cell surface. In some aspects, the at least one antigen-encoding nucleic acid sequences when administered to the subject and translated, the CTA-associated MHC class I epitope is presented on antigen presenting cells resulting in an immune response targeting CTA-associated MHC class I epitope on a tumor cell surface, and optionally wherein the expression of each of the at least one antigen-encoding nucleic acid sequences is driven by the at least one promoter nucleotide sequence.

In some aspects, each epitope-encoding nucleic acid sequence encodes a polypeptide sequence between 8 and 35 amino acids in length, optionally 9-17, 9-25, 8, 9, 10, 11, 12, 13, 14, 15, 16, 17, 18, 19, 20, 21, 22, 23, 24, 25, 26, 27, 28, 29, 30, 31, 32, 33, 34 or 35 amino acids in length.

In some aspects, the at least one MHC class II epitope-encoding nucleic acid sequence is present. In some aspects, the at least one MHC class II epitope-encoding nucleic acid sequence is present and comprises at least one MHC class II epitope-encoding nucleic acid sequence that comprises at least one alteration that makes the encoded peptide sequence distinct from the corresponding peptide sequence encoded by a wild-type nucleic acid sequence. In some aspects, the at least one MHC class II epitope-encoding nucleic acid sequence is 12-20, 12, 13, 14, 15, 16, 17, 18, 19, 20, or 20-40 amino acids in length. In some aspects, the at least one MHC class II epitope-encoding nucleic acid sequence is present and comprises at least one universal MHC class II antigen-encoding nucleic acid sequence, optionally wherein the at least one universal sequence comprises at least one of Tetanus toxoid and PADRE.

In some aspects, the at least one promoter nucleotide sequence or the second promoter nucleotide sequence is inducible. In some aspects, the at least one promoter nucleotide sequence or the second promoter nucleotide sequence is non-inducible.

In some aspects, the at least one poly(A) sequence comprises a poly(A) sequence native to the backbone. In some aspects, the at least one poly(A) sequence comprises a poly(A) sequence exogenous to the backbone. In some aspects, the at least one poly(A) sequence is operably linked to at least one of the at least one antigen-encoding nucleic acid sequences. In some aspects, the at least one poly(A) sequence is at least 20, at least 30, at least 40, at least 50, at least 60, at least 70, at least 80, or at least 90 consecutive A nucleotides. In some aspects, the at least one poly(A) sequence is at least 100 consecutive A nucleotides.

In some aspects, the cassette further comprises at least one of: an intron sequence, a woodchuck hepatitis virus posttranscriptional regulatory element (WPRE) sequence, an internal ribosome entry sequence (IRES) sequence, a nucleotide sequence encoding a 2A self-cleaving peptide sequence, a nucleotide sequence encoding a Furin cleavage site, or a sequence in the 5′ or 3′ non-coding region known to enhance the nuclear export, stability, or translation efficiency of mRNA that is operably linked to at least one of the at least one antigen-encoding nucleic acid sequences. In some aspects, the cassette further comprises a reporter gene, including but not limited to, green fluorescent protein (GFP), a GFP variant, secreted alkaline phosphatase, luciferase, a luciferase variant, or a detectable peptide or epitope. In some aspects, the detectable peptide or epitope is selected from the group consisting of an HA tag, a Flag tag, a His-tag, or a V5 tag.

In some aspects, the one or more vectors further comprises one or more nucleic acid sequences encoding at least one immune modulator. In some aspects, the immune modulator is an anti-CTLA4 antibody or an antigen-binding fragment thereof, an anti-PD-1 antibody or an antigen-binding fragment thereof, an anti-PD-L1 antibody or an antigen-binding fragment thereof, an anti-4-1BB antibody or an antigen-binding fragment thereof, or an anti-OX-40 antibody or an antigen-binding fragment thereof. In some aspects, the antibody or antigen-binding fragment thereof is a Fab fragment, a Fab′ fragment, a single chain Fv (scFv), a single domain antibody (sdAb) either as single specific or multiple specificities linked together (e.g., camelid antibody domains), or full-length single-chain antibody (e.g., full-length IgG with heavy and light chains linked by a flexible linker). In some aspects, the heavy and light chain sequences of the antibody are a contiguous sequence separated by either a self-cleaving sequence such as 2A or IRES; or the heavy and light chain sequences of the antibody are linked by a flexible linker such as consecutive glycine residues. In some aspects, the immune modulator is a cytokine. In some aspects, the cytokine is at least one of IL-2, IL-7, IL-12, IL-15, or IL-21 or variants thereof of each.

In some aspects, at least one epitope-encoding nucleic acid sequence is selected by performing the steps of: (a) obtaining at least one of exome, transcriptome, or whole genome nucleotide sequencing data from a tumor, wherein the nucleotide sequencing data is used to obtain data representing peptide sequences of each of a set of antigens; (b) inputting the peptide sequence of each antigen into a presentation model to generate a set of numerical likelihoods that each of the antigens is presented by one or more of the MHC alleles on a cell surface, optionally a tumor cell surface, the set of numerical likelihoods having been identified at least based on received mass spectrometry data; and (c) selecting a subset of the set of antigens based on the set of numerical likelihoods to generate a set of selected antigens which are used to generate the at least one epitope-encoding nucleic acid sequence.

In some aspects, each of the epitope-encoding nucleic acid sequences is selected by performing the steps of: (a) obtaining at least one of exome, transcriptome, or whole genome nucleotide sequencing data from a tumor, wherein the nucleotide sequencing data is used to obtain data representing peptide sequences of each of a set of antigens; (b) inputting the peptide sequence of each antigen into a presentation model to generate a set of numerical likelihoods that each of the antigens is presented by one or more of the MHC alleles on a cell surface, optionally a tumor cell surface, the set of numerical likelihoods having been identified at least based on received mass spectrometry data; and (c) selecting a subset of the set of antigens based on the set of numerical likelihoods to generate a set of selected antigens which are used to generate the at least 20 epitope-encoding nucleic acid sequences. In some aspects, a number of the set of selected antigens is 2-20. In some aspects, the presentation model represents dependence between: (a) presence of a pair of a particular one of the MHC alleles and a particular amino acid at a particular position of a peptide sequence; and (b) likelihood of presentation on a cell surface, optionally a tumor cell surface, by the particular one of the MHC alleles of the pair, of such a peptide sequence comprising the particular amino acid at the particular position. In some aspects, selecting the set of selected antigens comprises selecting antigens that have an increased likelihood of being presented on the cell surface relative to unselected antigens based on the presentation model, optionally wherein the selected antigens have been validated as being presented by one or more specific HLA alleles. In some aspects, selecting the set of selected antigens comprises selecting antigens that have an increased likelihood of being capable of inducing a tumor-specific or infectious disease-specific immune response in the subject relative to unselected antigens based on the presentation model. In some aspects, selecting the set of selected antigens comprises selecting antigens that have an increased likelihood of being capable of being presented to naïve T cells by professional antigen presenting cells (APCs) relative to unselected antigens based on the presentation model, optionally wherein the APC is a dendritic cell (DC). In some aspects, selecting the set of selected antigens comprises selecting antigens that have a decreased likelihood of being subject to inhibition via central or peripheral tolerance relative to unselected antigens based on the presentation model. In some aspects, selecting the set of selected antigens comprises selecting antigens that have a decreased likelihood of being capable of inducing an autoimmune response to normal tissue in the subject relative to unselected antigens based on the presentation model. In some aspects, exome or transcriptome nucleotide sequencing data is obtained by performing sequencing on a tumor cell or tissue. In some aspects, the sequencing is next generation sequencing (NGS) or any massively parallel sequencing approach.

In some aspects, the cassette comprises junctional epitope sequences formed by adjacent sequences in the cassette. In some aspects, at least one or each junctional epitope sequence has an affinity of greater than 500 nM for MHC. In some aspects, each junctional epitope sequence is non-self.

In some aspects, each of the MHC class I epitopes is predicted or validated to be capable of presentation by at least one HLA allele present in at least 5% of a population. In some aspects, each of the MHC class I epitopes is predicted or validated to be capable of presentation by at least one HLA allele, wherein each antigen/HLA pair has an antigen/HLA prevalence of at least 0.01% in a population. In some aspects, each of the MHC class I epitopes is predicted or validated to be capable of presentation by at least one HLA allele, wherein each antigen/HLA pair has an antigen/HLA prevalence of at least 0.1% in a population. In some aspects, The composition of any one of the above claims, wherein the CTA-associated MHC class I epitope is validated to be capable of presentation by at least one HLA allele, wherein each antigen/HLA pair has an antigen/HLA prevalence of at least 0.1% in a population. In some aspects, the CTA-associated MHC class I epitope is validated to be capable of presentation by at least one HLA allele, wherein each antigen/HLA pair has an antigen/HLA prevalence of at least 0.5% in a population. In some aspects, each of the CTA-associated MHC class I epitope is validated to be capable of presentation by at least one HLA allele, wherein each antigen/HLA pair has an antigen/HLA prevalence of at least 0.1% in a population. In some aspects, each of the CTA-associated MHC class I epitope is validated to be capable of presentation by at least one HLA allele, wherein each antigen/HLA pair has an antigen/HLA prevalence of at least 0.5% in a population. In some aspects, the at least one HLA allele is HLA A*01:01, HLA A*02:01, B*44:02, B*44:05, B*40:01, B*40:02, B*41:02, B*35:01, B*15:01, A*33:03, A*02:05, A*11:01, C*03:04, A*29:02, C*15:02, and/or B*07:02.

In some aspects, the cassette does not encode a non-therapeutic MHC class I or class II epitope nucleic acid sequence comprising a translated, wild-type nucleic acid sequence, wherein the non-therapeutic epitope is predicted to be displayed on an MHC allele of the subject. In some aspects, the non-therapeutic predicted MHC class I or class II epitope sequence is a junctional epitope sequence formed by adjacent sequences in the cassette.

In some aspects, the prediction is based on presentation likelihoods generated by inputting sequences of the non-therapeutic epitopes into a presentation model.

In some aspects, an order of the at least one antigen-encoding nucleic acid sequences in the cassette is determined by a series of steps comprising: (a) generating a set of candidate cassette sequences corresponding to different orders of the at least one antigen-encoding nucleic acid sequences; (b) determining, for each candidate cassette sequence, a presentation score based on presentation of non-therapeutic epitopes in the candidate cassette sequence; and (c) selecting a candidate cassette sequence associated with a presentation score below a predetermined threshold as the cassette sequence for an antigen vaccine.

Also provided for herein is a pharmaceutical composition comprising any of the compositions described herein and a pharmaceutically acceptable carrier. In some aspects, the composition further comprises an adjuvant. In some aspects, the composition further comprises an immune modulator. In some aspects, the immune modulator is an anti-CTLA4 antibody or an antigen-binding fragment thereof, an anti-PD-1 antibody or an antigen-binding fragment thereof, an anti-PD-L1 antibody or an antigen-binding fragment thereof, an anti-4-1BB antibody or an antigen-binding fragment thereof, or an anti-OX-40 antibody or an antigen-binding fragment thereof.

Also provided for herein is an isolated nucleotide sequence or set of isolated nucleotide sequences comprising the cassette of any of the compositions described herein and one or more elements obtained from the sequence of SEQ ID NO:3 or SEQ ID NO:5, optionally wherein the one or more elements are selected from the group consisting of the sequences necessary for nonstructural protein-mediated amplification, the 26S promoter nucleotide sequence, the poly(A) sequence, and the nsP1-4 genes of the sequence set forth in SEQ ID NO:3 or SEQ ID NO:5, and optionally wherein the nucleotide sequence is cDNA. In some aspects, the sequence or set of isolated nucleotide sequences comprises the cassette of any of the above composition claims inserted at position 7544 of the sequence set forth in SEQ ID NO:6 or SEQ ID NO:7. In some aspects, the composition further comprises: a) a T7 or SP6 RNA polymerase promoter nucleotide sequence 5′ of the one or more elements obtained from the sequence of SEQ ID NO:3 or SEQ ID NO:5; and b) optionally, one or more restriction sites 3′ of the poly(A) sequence. In some aspects, the cassette of any of the above composition claims is inserted at position 7563 of SEQ ID NO:8 or SEQ ID NO:9.

Also provided for herein is a vector or set of vectors comprising any of the nucleotide sequence described herein.

Also provided for herein is an isolated cell comprising any of the nucleotide sequences or set of isolated nucleotide sequences described herein, optionally wherein the cell is a BHK-21, CHO, HEK293 or variants thereof, 911, HeLa, A549, LP-293, PER.C6, or AE1-2a cell.

Also provided for herein is a kit comprising any of the compositions described herein and instructions for use.

In some aspects, any of the above compositions further comprise a nanoparticulate delivery vehicle. The nanoparticulate delivery vehicle, in some aspects, may be a lipid nanoparticle (LNP). In some aspects, the LNP comprises ionizable amino lipids. In some aspects, the ionizable amino lipids comprise MC3-like (dilinoleylmethyl-4-dimethylaminobutyrate) molecules. In some aspects, the nanoparticulate delivery vehicle encapsulates the antigen expression system.

In some aspects, any of the above compositions further comprise a plurality of LNPs, wherein the LNPs comprise: the antigen expression system; a cationic lipid; a non-cationic lipid; and a conjugated lipid that inhibits aggregation of the LNPs, wherein at least about 95% of the LNPs in the plurality of LNPs either: have a non-lamellar morphology; or are electron-dense.

In some aspects, the non-cationic lipid is a mixture of (1) a phospholipid and (2) cholesterol or a cholesterol derivative.

In some aspects, the conjugated lipid that inhibits aggregation of the LNPs is a polyethyleneglycol (PEG)-lipid conjugate. In some aspects, the PEG-lipid conjugate is selected from the group consisting of: a PEG-diacylglycerol (PEG-DAG) conjugate, a PEG dialkyloxypropyl (PEG-DAA) conjugate, a PEG-phospholipid conjugate, a PEG-ceramide (PEG-Cer) conjugate, and a mixture thereof. In some aspects the PEG-DAA conjugate is a member selected from the group consisting of: a PEG-didecyloxypropyl (C₁₀) conjugate, a PEG-dilauryloxypropyl (C₁₂) conjugate, a PEG-dimyristyloxypropyl (C₁₄) conjugate, a PEG-dipalmityloxypropyl (C₁₆) conjugate, a PEG-distearyloxypropyl (Cis) conjugate, and a mixture thereof.

In some aspects, the antigen expression system is fully encapsulated in the LNPs.

In some aspects, the non-lamellar morphology of the LNPs comprises an inverse hexagonal (Hₗₗ) or cubic phase structure.

In some aspects, the cationic lipid comprises from about 10 mol % to about 50 mol % of the total lipid present in the LNPs. In some aspects, the cationic lipid comprises from about 20 mol % to about 50 mol % of the total lipid present in the LNPs. In some aspects, the cationic lipid comprises from about 20 mol % to about 40 mol % of the total lipid present in the LNPs.

In some aspects, the non-cationic lipid comprises from about 10 mol % to about 60 mol % of the total lipid present in the LNPs. In some aspects, the non-cationic lipid comprises from about 20 mol % to about 55 mol % of the total lipid present in the LNPs. In some aspects, the non-cationic lipid comprises from about 25 mol % to about 50 mol % of the total lipid present in the LNPs.

In some aspects, the conjugated lipid comprises from about 0.5 mol % to about 20 mol % of the total lipid present in the LNPs. In some aspects, the conjugated lipid comprises from about 2 mol % to about 20 mol % of the total lipid present in the LNPs. In some aspects, the conjugated lipid comprises from about 1.5 mol % to about 18 mol % of the total lipid present in the LNPs.

In some aspects, greater than 95% of the LNPs have a non-lamellar morphology. In some aspects, greater than 95% of the LNPs are electron dense.

In some aspects, any of the above compositions further comprise a plurality of LNPs, wherein the LNPs comprise: a cationic lipid comprising from 50 mol % to 65 mol % of the total lipid present in the LNPs; a conjugated lipid that inhibits aggregation of LNPs comprising from 0.5 mol % to 2 mol % of the total lipid present in the LNPs; and a non-cationic lipid comprising either: a mixture of a phospholipid and cholesterol or a derivative thereof, wherein the phospholipid comprises from 4 mol % to 10 mol % of the total lipid present in the LNPs and the cholesterol or derivative thereof comprises from 30 mol % to 40 mol % of the total lipid present in the LNPs; a mixture of a phospholipid and cholesterol or a derivative thereof, wherein the phospholipid comprises from 3 mol % to 15 mol % of the total lipid present in the LNPs and the cholesterol or derivative thereof comprises from 30 mol % to 40 mol % of the total lipid present in the LNPs; or up to 49.5 mol % of the total lipid present in the LNPs and comprising a mixture of a phospholipid and cholesterol or a derivative thereof, wherein the cholesterol or derivative thereof comprises from 30 mol % to 40 mol % of the total lipid present in the LNPs.

In some aspects, any of the above compositions further comprise a plurality of LNPs, wherein the LNPs comprise: a cationic lipid comprising from 50 mol % to 85 mol % of the total lipid present in the LNPs; a conjugated lipid that inhibits aggregation of LNPs comprising from 0.5 mol % to 2 mol % of the total lipid present in the LNPs; and a non-cationic lipid comprising from 13 mol % to 49.5 mol % of the total lipid present in the LNPs.

In some aspects, the phospholipid comprises dipalmitoylphosphatidylcholine (DPPC), distearoylphosphatidylcholine (DSPC), or a mixture thereof.

In some aspects, the conjugated lipid comprises a polyethyleneglycol (PEG)-lipid conjugate. In some aspects, the PEG-lipid conjugate comprises a PEG-diacylglycerol (PEG-DAG) conjugate, a PEG-dialkyloxypropyl (PEG-DAA) conjugate, or a mixture thereof. In some aspects, the PEG-DAA conjugate comprises a PEG-dimyristyloxypropyl (PEG-DMA) conjugate, a PEG-distearyloxypropyl (PEG-DSA) conjugate, or a mixture thereof. In some aspects, the PEG portion of the conjugate has an average molecular weight of about 2,000 daltons.

In some aspects, the conjugated lipid comprises from 1 mol % to 2 mol % of the total lipid present in the LNPs.

In some aspects, the LNP comprises a compound having a structure of Formula I:

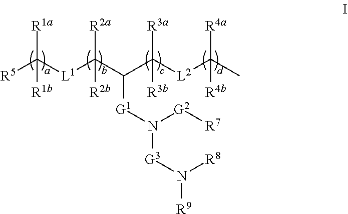

or a pharmaceutically acceptable salt, tautomer, prodrug or stereoisomer thereof, wherein: L¹ and L² are each independently -0(C=0)-, —(C=0)0-, —C(=0)-, -0-, —S(0)ₓ-, —S—S—, —C(=0)S—, —SC(=0)-, —R^{a}C(=0)-, —C(=0) R^{a}—, —R^{a}C(=0) R^{a}—, —OC(=0) R^{a}—, —R^{a}C(=0)0- or a direct bond; G¹ is Ci-C₂ alkylene, —(C=0)-, -0(C=0)-, —SC(=0)-, —R^{a}C(=0)- or a direct bond: —C(=0)-, —(C=0)0-, —C(=0)S—, —C(=0) R^{a}— or a direct bond; G is Ci-C₆ alkylene; R^{a} is H or C1-C12 alkyl; R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond; R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond; R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond; R^{4a} and R^{4b} are, at each occurrence, independently either: (a) H or C1-C12 alkyl; or (b) R^{4a} is H or C1-C12 alkyl, and R^{4b} together with the carbon atom to which it is bound is taken together with an adjacent R^{4b} and the carbon atom to which it is bound to form a carbon-carbon double bond; R⁵ and R⁶ are each independently H or methyl; R⁷ is C4-C20 alkyl; R⁸ and R⁹ are each independently C1-C12 alkyl; or R⁸ and R⁹, together with the nitrogen atom to which they are attached, form a 5, 6 or 7-membered heterocyclic ring; a, b, c and d are each independently an integer from 1 to 24; and x is 0, 1 or 2.

In some aspects, the LNP comprises a compound having a structure of Formula II:

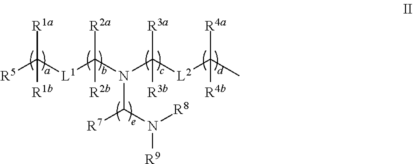

or a pharmaceutically acceptable salt, tautomer, prodrug or stereoisomer thereof, wherein: L¹ and L2 are each independently -0(C=0)-, —(C=0)0- or a carbon-carbon double bond; R^{1a} and R^{1b} are, at each occurrence, independently either (a) H or C1-C12 alkyl, or (b) R^{1a} is H or C1-C12 alkyl, and R^{1b} together with the carbon atom to which it is bound is taken together with an adjacent R^{1b} and the carbon atom to which it is bound to form a carbon-carbon double bond; R^{2a} and R^{2b} are, at each occurrence, independently either (a) H or C1-C12 alkyl, or (b) R^{2a} is H or C1-C12 alkyl, and R^{2b} together with the carbon atom to which it is bound is taken together with an adjacent R^{2b} and the carbon atom to which it is bound to form a carbon-carbon double bond; R^{3a} and R^{3b} are, at each occurrence, independently either (a) H or C1-C12 alkyl, or (b) R^{3a} is H or C1-C12 alkyl, and R^{3b} together with the carbon atom to which it is bound is taken together with an adjacent R^{3b} and the carbon atom to which it is bound to form a carbon-carbon double bond; R^{4a} and R^{4b} are, at each occurrence, independently either (a) H or C1-C12 alkyl, or (b) R^{4a} is H or C1-C12 alkyl, and R^{4b} together with the carbon atom to which it is bound is taken together with an adjacent R^{4b} and the carbon atom to which it is bound to form a carbon-carbon double bond; R⁵ and R⁶ are each independently methyl or cycloalkyl; R⁷ is, at each occurrence, independently H or C1-C12 alkyl; R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom; a and d are each independently an integer from 0 to 24; b and c are each independently an integer from 1 to 24; and e is 1 or 2, provided that: at least one of R^{1a}, R^{2a}, R^{3a} or R^{4a} is C1-C12 alkyl, or at least one of L¹ or L² is -0(C=0)- or —(C=0)0-; and R^{1a} and R^{1b} are not isopropyl when a is 6 or n-butyl when a is 8.

In some aspects, any of the above compositions further comprise one or more excipients comprising a neutral lipid, a steroid, and a polymer conjugated lipid. In some aspects, the neutral lipid comprises at least one of 1,2-Distearoyl-sn-glycero-3-phosphocholine (DSPC), 1,2-Dipalmitoyl-sn-glycero-3-phosphocholine (DPPC), 1,2-Dimyristoyl-sn-glycero-3-phosphocholine (DMPC), 1-Palmitoyl-2-oleoyl-sn-glycero-3-phosphocholine (POPC), 1,2-dioleoyl-sn-glycero-3-phosphocholine (DOPC), and 1,2-Dioleoyl-sn-glycero-3-phosphoethanolamine (DOPE). In some aspects, the neutral lipid is DSPC.

In some aspects, the molar ratio of the compound to the neutral lipid ranges from about 2:1 to about 8:1.

In some aspects, the steroid is cholesterol. In some aspects, the molar ratio of the compound to cholesterol ranges from about 2:1 to 1:1.

In some aspects, the polymer conjugated lipid is a pegylated lipid. In some aspects, the molar ratio of the compound to the pegylated lipid ranges from about 100:1 to about 25:1. In some aspects, the pegylated lipid is PEG-DAG, a PEG polyethylene (PEG-PE), a PEG-succinoyl-diacylglycerol (PEG-S-DAG), PEG-cer or a PEG dialkyoxypropylcarbamate. In some aspects, the pegylated lipid has the following structure III:

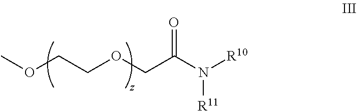

or a pharmaceutically acceptable salt, tautomer or stereoisomer thereof, wherein: R¹⁰ and R¹¹ are each independently a straight or branched, saturated or unsaturated alkyl chain containing from 10 to 30 carbon atoms, wherein the alkyl chain is optionally interrupted by one or more ester bonds; and z has a mean value ranging from 30 to 60. In some aspects, R¹⁰ and R¹¹ are each independently straight, saturated alkyl chains having 12 to 16 carbon atoms. In some aspects, the average z is about 45.
start here

In some aspects, the LNP self-assembles into non-bilayer structures when mixed with polyanionic nucleic acid. In some aspects, the non-bilayer structures have a diameter between 60 nm and 120 nm. In some aspects, the non-bilayer structures have a diameter of about 70 nm, about 80 nm, about 90 nm, or about 100 nm. In some aspects, wherein the nanoparticulate delivery vehicle has a diameter of about 100 nm.

Also provided for herein is a method for treating a subject with cancer, the method comprising administering to the subject any of the compositions or any of the pharmaceutical compositions described herein. In some aspects, the at least one epitope-encoding nucleic acid sequence is derived from the tumor of the subject with cancer or from a cell or sample of the infected subject. In some aspects, the at least one epitope-encoding nucleic acid sequence are not derived from the tumor of the subject with cancer or from a cell or sample of the infected subject.

Also provided for herein is a method for stimulating an immune response in a subject, the method comprising administering to the subject any of the compositions or any of the pharmaceutical compositions described herein.

In some aspects, the subject expresses at least one HLA allele predicted or known to present the MHC class I epitope. In some aspects, the composition is administered intramuscularly (IM), intradermally (ID), subcutaneously (SC), or intravenously (IV). In some aspects, the composition is administered intramuscularly. In some aspects, the method further comprising administration of one or more immune modulators, optionally wherein the immune modulator is administered before, concurrently with, or after administration of the composition or pharmaceutical composition. In some aspects, the one or more immune modulators are selected from the group consisting of: an anti-CTLA4 antibody or an antigen-binding fragment thereof, an anti-PD-1 antibody or an antigen-binding fragment thereof, an anti-PD-L1 antibody or an antigen-binding fragment thereof, an anti-4-1BB antibody or an antigen-binding fragment thereof, or an anti-OX-40 antibody or an antigen-binding fragment thereof. In some aspects, the immune modulator is administered intravenously (IV), intramuscularly (IM), intradermally (ID), or subcutaneously (SC). In some aspects, the subcutaneous administration is near the site of the composition or pharmaceutical composition administration or in close proximity to one or more vector or composition draining lymph nodes.

In some aspects, the method further comprises administering to the subject a second vaccine composition. In some aspects, the second vaccine composition is administered prior to the administration of any of the compositions or the pharmaceutical compositions described herein. In some aspects, the second vaccine composition is administered subsequent to the administration of any of the compositions or the pharmaceutical compositions described herein. In some aspects, the second vaccine composition is the same as any of the compositions or the pharmaceutical compositions described herein. In some aspects, the second vaccine composition is different any of the compositions or the pharmaceutical compositions described herein. In some aspects, the second vaccine composition comprises a chimpanzee adenovirus vector encoding at least one antigen-encoding nucleic acid sequence. In some aspects, the at least one antigen-encoding nucleic acid sequence encoded by the chimpanzee adenovirus vector is the same as the at least one antigen-encoding nucleic acid sequence of any of the above composition claims.

Also provided for herein is a method of manufacturing the one or more vectors of any of the above composition claims, the method comprising: (a) obtaining a linearized DNA sequence comprising the backbone and the cassette; (b) in vitro transcribing the linearized DNA sequence by addition of the linearized DNA sequence to an in vitro transcription reaction containing all the necessary components to transcribe the linearized DNA sequence into RNA, optionally further comprising in vitro addition of the m7 g cap to the resulting RNA; and (c) isolating the one or more vectors from the in vitro transcription reaction. In some aspects, the linearized DNA sequence is generated by linearizing a DNA plasmid sequence or by amplification using PCR. In some aspects, the DNA plasmid sequence is generated using one of bacterial recombination or full genome DNA synthesis or full genome DNA synthesis with amplification of synthesized DNA in bacterial cells. In some aspects, isolating the one or more vectors from the in vitro transcription reaction involves one or more of phenol chloroform extraction, silica column based purification, or similar RNA purification methods.

Also provided for herein is a method of manufacturing the composition of any of the above composition claims for delivery of the antigen expression system, the method comprising: (a) providing components for the nanoparticulate delivery vehicle; (b) providing the antigen expression system; and (c) providing conditions sufficient for the nanoparticulate delivery vehicle and the antigen expression system to produce the composition for delivery of the antigen expression system. In some aspects, the conditions are provided by microfluidic mixing.

Also provided for herein is a method for treating a subject with a disease, optionally wherein the disease is cancer or an infection, the method comprising administering to the subject an antigen-based vaccine to the subject, wherein the antigen-based vaccine comprises an antigen-encoding cassette, or a polypeptide sequence encoded by the cassette, wherein the antigen-encoding cassette comprises at least one antigen-encoding nucleic acid sequence described, from 5′ to 3′, by the formula:

(Eₓ-(E^{N}ₙ)_{y})_{z}

wherein E represents a nucleotide sequence comprising a distinct epitope-encoding nucleic acid sequence, n represents the number of separate distinct epitope-encoding nucleic acid sequences and is any integer including 0, E^{N} represents a nucleotide sequence comprising the separate distinct epitope-encoding nucleic acid sequence for each corresponding n, for each iteration of z: x=0 or 1, y=0 or 1 for each n, and at least one of x or y=1, and z=1 or greater, optionally wherein the antigen-encoding nucleic acid sequence comprises at least two iterations of E, a given E^{N}, or a combination thereof, and at least one of the distinct epitope-encoding nucleic acid sequences encodes a distinct shared Cancer Testis Antigen (CTA)-associated MHC class I epitope, optionally wherein at least one of the distinct epitope-encoding nucleic acid sequences encoding the CTA comprises at least two iterations.

Also provided for herein is a method for treating a subject with a disease, optionally wherein the disease is cancer, the method comprising administering to the subject an antigen-based vaccine to the subject, wherein the antigen-based vaccine comprises an antigen expression system, comprising: the antigen expression system, wherein the antigen expression system comprises one or more vectors, the one or more vectors comprising: (a) a vector backbone, wherein the backbone comprises: (i) at least one promoter nucleotide sequence, and (ii) optionally, at least one polyadenylation (poly(A)) sequence; and (b) a cassette, wherein the cassette comprises: (i) at least one antigen-encoding nucleic acid sequence, comprising: (I) at least one epitope-encoding nucleic acid sequence encoding a CTA-associated MHC class I epitope, and wherein each of the epitope-encoding nucleic acid sequences comprises; (A) optionally, a 5′ linker sequence, and (B) optionally, a 3′ linker sequence; (ii) optionally, a second promoter nucleotide sequence operably linked to the antigen-encoding nucleic acid sequence; and (iii) optionally, at least one MHC class II epitope-encoding nucleic acid sequence; (iv) optionally, at least one nucleic acid sequence encoding a GPGPG amino acid linker sequence (SEQ ID NO:56); and (v) optionally, at least one second poly(A) sequence, wherein the second poly(A) sequence is a native poly(A) sequence or an exogenous poly(A) sequence to the vector backbone, wherein if the second promoter nucleotide sequence is absent, the antigen-encoding nucleic acid sequence is operably linked to the at least one promoter nucleotide sequence, and wherein the at least one antigen-encoding nucleic acid sequence comprises at least two iterations of the epitope-encoding nucleic acid sequence encoding the CTA-associated MHC class I epitope.

In some aspects, the at least one epitope-encoding nucleic acid sequence is derived from a tumor of the subject with cancer. In some aspects, the at least one epitope-encoding nucleic acid sequence are not derived from a tumor of the subject with cancer.

Also provided for herein is a method for stimulating an immune response in a subject, the method comprising the method comprising administering to the subject an antigen-based vaccine to the subject, wherein the antigen-based vaccine comprises an antigen-encoding cassette, or a polypeptide sequence encoded by the cassette, wherein the antigen-encoding cassette comprises at least one antigen-encoding nucleic acid sequence described, from 5′ to 3′, by the formula:

(Eₓ-(E^{N}ₙ)_{y})_{z}

wherein E represents a nucleotide sequence comprising a distinct epitope-encoding nucleic acid sequence, n represents the number of separate distinct epitope-encoding nucleic acid sequences and is any integer including 0, E^{N} represents a nucleotide sequence comprising the separate distinct epitope-encoding nucleic acid sequence for each corresponding n, for each iteration of z: x=0 or 1, y=0 or 1 for each n, and at least one of x or y=1, and z=1 or greater, optionally wherein the antigen-encoding nucleic acid sequence comprises at least two iterations of E, a given E^{N}, or a combination thereof, and at least one of the distinct epitope-encoding nucleic acid sequences encodes a distinct shared Cancer Testis Antigen (CTA)-associated MHC class I epitope, optionally wherein at least one of the distinct epitope-encoding nucleic acid sequences encoding the CTA comprises at least two iterations.

Also provided for herein is a method for stimulating an immune response in a subject, the method comprising the method comprising administering to the subject an antigen-based vaccine to the subject, wherein the antigen-based vaccine comprises: an antigen expression system, comprising: the antigen expression system, wherein the antigen expression system comprises one or more vectors, the one or more vectors comprising: (a) a vector backbone, wherein the backbone comprises: (i) at least one promoter nucleotide sequence, and (ii) optionally, at least one polyadenylation (poly(A)) sequence; and (b) a cassette, wherein the cassette comprises: (i) at least one antigen-encoding nucleic acid sequence, comprising: (I) at least one epitope-encoding nucleic acid sequence encoding a CTA-associated MHC class I epitope, and wherein each of the epitope-encoding nucleic acid sequences comprises; (A) optionally, a 5′ linker sequence, and (B) optionally, a 3′ linker sequence; (ii) optionally, a second promoter nucleotide sequence operably linked to the antigen-encoding nucleic acid sequence; and (iii) optionally, at least one MHC class II epitope-encoding nucleic acid sequence; (iv) optionally, at least one nucleic acid sequence encoding a GPGPG amino acid linker sequence (SEQ ID NO:56); and (v) optionally, at least one second poly(A) sequence, wherein the second poly(A) sequence is a native poly(A) sequence or an exogenous poly(A) sequence to the vector backbone, wherein if the second promoter nucleotide sequence is absent, the antigen-encoding nucleic acid sequence is operably linked to the at least one promoter nucleotide sequence, and wherein the at least one antigen-encoding nucleic acid sequence comprises at least two iterations of the epitope-encoding nucleic acid sequence encoding the CTA-associated MHC class I epitope.

In some aspects, the subject expresses at least one HLA allele predicted or known to present the CTA-associated MHC class I epitope. In some aspects, the subject expresses at least one HLA allele predicted or known to present the at least one epitope sequence, and wherein the at least one epitope sequence comprises an epitope known or suspected to be presented by MHC class I on a surface of a cell, wherein the at least one epitope sequence predicted or known to be presented comprises the CTA-associated MHC class I epitope. In some aspects, the surface of the cell is a tumor cell surface. In some aspects, the cell is a tumor cell selected from the group consisting of: lung cancer, melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, T cell lymphocytic leukemia, non-small cell lung cancer, and small cell lung cancer. In some aspects, the cell is a lung adenocarcinoma, ovarian serous cystadenocarcinoma, lung squamous cell carcinoma, colon cancer, or head and neck squamous cell carcinoma tumor cell.

Also provided for herein is a method for inducing an immune response in a subject, the method comprising administering to the subject an antigen-based vaccine to the subject, wherein the antigen-based vaccine comprises an antigen-encoding cassette, or a polypeptide sequence encoded by the cassette, wherein the antigen-encoding cassette comprises at least one antigen-encoding nucleic acid sequence described, from 5′ to 3′, by the formula:

(Eₓ-(E^{N}ₙ)_{y})_{z}

wherein E represents a nucleotide sequence comprising a distinct epitope-encoding nucleic acid sequence, n represents the number of separate distinct epitope-encoding nucleic acid sequences and is any integer including 0, E^{N} represents a nucleotide sequence comprising the separate distinct epitope-encoding nucleic acid sequence for each corresponding n, for each iteration of z: x=0 or 1, y=0 or 1 for each n, and at least one of x or y=1, and z=1 or greater, optionally wherein the antigen-encoding nucleic acid sequence comprises at least two iterations of E, a given E^{N}, or a combination thereof, and at least one of the distinct epitope-encoding nucleic acid sequences encodes a distinct shared Cancer Testis Antigen (CTA)-associated MHC class I epitope, optionally wherein at least one of the distinct epitope-encoding nucleic acid sequences encoding the CTA comprises at least two iterations, and wherein the subject expresses at least one HLA allele predicted or known to present the at least one CTA-associated MHC class I epitope.

Also provided for herein is a method for inducing an immune response in a subject, the method comprising administering to the subject an antigen-based vaccine to the subject, wherein the antigen-based vaccine comprises: an antigen expression system, comprising: the antigen expression system, wherein the antigen expression system comprises one or more vectors, the one or more vectors comprising: (a) a vector backbone, wherein the backbone comprises: (i) at least one promoter nucleotide sequence, and (ii) optionally, at least one polyadenylation (poly(A)) sequence; and (b) a cassette, wherein the cassette comprises: (i) at least one antigen-encoding nucleic acid sequence, comprising: (I) at least one epitope-encoding nucleic acid sequence encoding a CTA-associated MHC class I epitope, and wherein each of the epitope-encoding nucleic acid sequences comprises; (A) optionally, a 5′ linker sequence, and (B) optionally, a 3′ linker sequence; (ii) optionally, a second promoter nucleotide sequence operably linked to the antigen-encoding nucleic acid sequence; and (iii) optionally, at least one MHC class II epitope-encoding nucleic acid sequence; (iv) optionally, at least one nucleic acid sequence encoding a GPGPG amino acid linker sequence (SEQ ID NO:56); and (v) optionally, at least one second poly(A) sequence, wherein the second poly(A) sequence is a native poly(A) sequence or an exogenous poly(A) sequence to the vector backbone, wherein if the second promoter nucleotide sequence is absent, the antigen-encoding nucleic acid sequence is operably linked to the at least one promoter nucleotide sequence, wherein the at least one antigen-encoding nucleic acid sequence comprises at least two iterations of the epitope-encoding nucleic acid sequence encoding the CTA-associated MHC class I epitope, and wherein the subject expresses at least one HLA allele predicted or known to present the at least one CTA-associated MHC class I epitope.

In some aspects, the antigen-encoding cassette comprises encodes the amino acid sequence SEQ ID NO: 10,779. In some aspects, the antigen-encoding cassette comprises encodes the amino acid sequence SEQ ID NO: 10,781. In some aspects, the antigen-encoding cassette comprises encodes the amino acid sequence SEQ ID NO: 10,783. In some aspects, the antigen-encoding cassette comprises encodes the amino acid sequence SEQ ID NO: 10,785. In some aspects, the antigen-encoding cassette comprises encodes the amino acid sequence SEQ ID NO: 10,787.

In some aspects, the at least one promoter sequence is a CMV, SV40, EF-1, RSV, PGK, HSA, MCK or EBV promoter sequence. In some aspects, the at least one promoter sequence is a regulatable promoter, optionally wherein the regulatable promoter is a tetracycline (TET) repressor protein (TETr) controlled promoter, optionally wherein the regulatable promoter comprises multiple TET operator (TETo) sequences 5′ or 3′ of a RNA polymerase binding sequence of the promoter. In some aspects, P comprises a CMV-derived promoter sequence, optionally wherein the CMV-derived promoter sequence comprises a TETr controlled CMV-derived promoter.

In some aspects, the antigen expression system comprises any one of the antigen expression systems described herein. In some aspects, the antigen-based vaccine comprises any one of the pharmaceutical compositions described herein.

In some aspects, the antigen-based vaccine is administered as a priming dose. In some aspects, the antigen-based vaccine is administered as one or more boosting doses. In some aspects, the boosting dose is different than the priming dose. In some aspects, a) the priming dose comprises a chimpanzee adenovirus vector and the boosting dose comprises an alphavirus vector; or b) the priming dose comprises an alphavirus vector and the boosting dose comprises a chimpanzee adenovirus vector. In some aspects, the boosting dose is the same as the priming dose. In some aspects, the injection site of the one or more boosting doses is as close as possible to the injection site of the priming dose.

In some aspects, the method further comprises determining or having determined the HLA-haplotype of the subject.

In some aspects, the antigen-based vaccine is administered intramuscularly (IM), intradermally (ID), subcutaneously (SC), or intravenously (IV). In some aspects, the antigen-based vaccine is administered intramuscularly (IM). In some aspects, the IM administration is administered at separate injection sites. In some aspects, the separate injection sites are in opposing deltoid muscles. In some aspects, the separate injection sites are in gluteus or rectus femoris sites on each side.

Also disclosed herein is a pharmaceutical composition comprising any of the compositions disclosed herein (such as an alphavirus-based or ChAd-based vector disclosed herein) and a pharmaceutically acceptable carrier. In some aspects, the pharmaceutical composition further comprises an adjuvant. In some aspects, the pharmaceutical composition further comprises an immune modulator. In some aspects, the immune modulator is an anti-CTLA4 antibody or an antigen-binding fragment thereof, an anti-PD-1 antibody or an antigen-binding fragment thereof, an anti-PD-L1 antibody or an antigen-binding fragment thereof, an anti-4-1BB antibody or an antigen-binding fragment thereof, or an anti-OX-40 antibody or an antigen-binding fragment thereof.

Also disclosed herein is a vector comprising an isolated nucleotide sequence disclosed herein.

Also disclosed herein is a kit comprising a vector or a composition disclosed herein and instructions for use.

Also disclosed herein is a method for treating a subject, the method comprising administering to the subject a vector disclosed herein or a pharmaceutical composition disclosed herein. Also disclosed herein is a method for inducing an immune response in a subject, the method comprising administering to the subject any of the compositions, vectors, or pharmaceutical compositions described herein. In some aspects, the subject expresses at least one HLA allele predicted or known to present the MHC class I epitope. In some aspects, the vector or composition is administered intramuscularly (IM), intradermally (ID), or subcutaneously (SC), or intravenously (IV).

Also disclosed herein is a method of manufacturing the one or more vectors of any of the above compositions, the method comprising: obtaining a linearized DNA sequence comprising the backbone and the antigen cassette; in vitro transcribing the linearized DNA sequence by addition of the linearized DNA sequence to a in vitro transcription reaction containing all the necessary components to transcribe the linearized DNA sequence into RNA, optionally further comprising in vitro addition of the m7 g cap to the resulting RNA; and isolating the one or more vectors from the in vitro transcription reaction. In some aspects, the linearized DNA sequence is generated by linearizing a DNA plasmid sequence or by amplification using PCR. In some aspects, the DNA plasmid sequence is generated using one of bacterial recombination or full genome DNA synthesis or full genome DNA synthesis with amplification of synthesized DNA in bacterial cells. In some aspects, the isolating the one or more vectors from the in vitro transcription reaction involves one or more of phenol chloroform extraction, silica column based purification, or similar RNA purification methods.

Also disclosed herein is a method of manufacturing any of the compositions disclosed herein, the method comprising: providing components for the nanoparticulate delivery vehicle; providing the antigen expression system; and providing conditions sufficient for the nanoparticulate delivery vehicle and the antigen expression system to produce the composition for delivery of the antigen expression system. In some aspects, the conditions are provided by microfluidic mixing.

Also disclosed herein is a method of manufacturing a adenovirus vector disclosed herein, the method comprising: obtaining a plasmid sequence comprising the at least one promoter sequence and the antigen cassette; transfecting the plasmid sequence into one or more host cells; and isolating the adenovirus vector from the one or more host cells.

In some aspects, isolating comprises: lysing the host cell to obtain a cell lysate comprising the adenovirus vector; and purifying the adenovirus vector from the cell lysate.

In some aspects, the plasmid sequence is generated using one of bacterial recombination or full genome DNA synthesis or full genome DNA synthesis with amplification of synthesized DNA in bacterial cells. In some aspects, the one or more host cells are at least one of CHO, HEK293 or variants thereof, 911, HeLa, A549, LP-293, PER.C6, and AE1-2a cells. In some aspects, purifying the adenovirus vector from the cell lysate involves one or more of chromatographic separation, centrifugation, virus precipitation, and filtration.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 provides a graphical illustration of the HLA coverage, shown as the number of TCE epitopes validated in tumors by mass spectrometry in each HLA group, (% US population with at least 1 allele shown in parentheses).

FIG. 2 shows population coverage for the Option 4 CTA cassette across various groups.

FIG. 3A illustrates that CTA and KRAS mutations have heterogenous co-expression in low PD-L1 lung tumors, indicating a higher likelihood for multiple antigen targets in vaccine strategies including both CTA and KRAS epitopes.

FIG. 3B illustrates various proposed constructs and vaccines combinations having single or iterated (repeated) CTA epitopes, with or without KRAS epitopes, including cassettes having iterated KRAS epitopes.

FIG. 4A presents target density analysis for the indicated CTA epitopes encoded by the various CTA cassettes for HLA-A*01:01.

FIG. 4B presents target density analysis for the indicated CTA epitopes encoded by the various CTA cassettes for HLA-A*02:01.

FIG. 4C presents target density analysis for the indicated CTA epitopes encoded by the various CTA cassettes for HLA-A*11:01.

FIG. 5 shows the vaccination strategy and results for HLA-A02:01 expressing mice immunized with ChAdV68 delivery vectors encoding CTA-encoding cassettes having various number of iterations of each CTA epitope, ranging from one to four iterations, as assessed by IFNg ELISpot.

FIG. 6 shows the vaccination strategy and results for HLA-A02:01 expressing mice immunized with ChAdV68 delivery vectors encoding (A) CTA 8×2 “Option 1” (column 1 “CTA”) or (B) CTA 8×2 “Option 1” and KRAS G12V/G12C “2×4” expressed as a single polypeptide with KRAS epitopes interspersed with CTA epitopes. (column 2 “CTA-KRAS”) and stimulated with the indicated predicted MAGE epitopes, as assessed by IFNg ELISpot.

FIG. 7 shows the vaccination strategy and results for HLA-A02:01 and HLA-A01:01 expressing mice immunized with ChAdV68 delivery vectors encoding single iterations of CTA “Option 4” linked to KRAS G12V & G12C “2×4” (column 1), two iterations of CTA “Option 4” linked to KRAS G12V/G12C “2×4” (column 2), single iterations of CTA “Option 4” linked to KRAS G12V & G12C “2×4” administered bilaterally in combination with KRAS “4×4” (column 3), or CTA 8×2 “Option 4” linked to KRAS G12V/G12C “2×4” administered bilaterally in combination with KRAS “4×4” (column 4), and stimulated with the indicated predicted MAGE or CT83 epitopes (left and middle panels, respectively), as assessed by IFNg ELISpot.

FIG. 8A shows the vaccination strategy and results for mice administered the bilateral or blended vaccines that included that 8×2 “Option 1” vaccines and KRAS 4×4 vaccines via ChAdV68 delivery vectors, as assessed by IFNg ELISpot for the indicated MAGE epitopes.

FIG. 8B shows the vaccination strategy and results for mice administered the bilateral or blended vaccines that included that 8×2 “Option 1” vaccines and KRAS 4×4 vaccines via samRNA vaccines, as assessed by IFNg ELISpot for the indicated MAGE epitopes.

FIG. 8C shows the vaccination strategy and results for mice administered the bilateral or blended vaccines that included that 8×2 “Option 1” vaccines and KRAS 4×4 vaccines via either ChAdV68 delivery vectors (left panel) or samRNA vaccines (right panel), as assessed by IFNg ELISpot for G12V T cell responses.

### DETAILED DESCRIPTION

### I. Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

As used herein the term “antigen” is a substance that induces an immune response. An antigen can be a neoantigen. An antigen can be a “shared antigen” that is an antigen found among a specific population, e.g., a specific population of cancer patients.

As used herein the term “neoantigen” is an antigen that has at least one alteration that makes it distinct from the corresponding wild-type antigen, e.g., via mutation in a tumor cell or post-translational modification specific to a tumor cell. A neoantigen can include a polypeptide sequence or a nucleotide sequence. A mutation can include a frameshift or nonframeshift indel, missense or nonsense substitution, splice site alteration, genomic rearrangement or gene fusion, or any genomic or expression alteration giving rise to a neoORF. A mutations can also include a splice variant. Post-translational modifications specific to a tumor cell can include aberrant phosphorylation. Post-translational modifications specific to a tumor cell can also include a proteasome-generated spliced antigen. See Liepe et al., A large fraction of HLA class I ligands are proteasome-generated spliced peptides; Science. 2016 Oct. 21; 354(6310):354-358. The subject can be identified for administration through the use of various diagnostic methods, e.g., patient selection methods described further below.

As used herein the term “tumor antigen” is an antigen present in a subject's tumor cell or tissue but not in the subject's corresponding normal cell or tissue, or derived from a polypeptide known to or have been found to have altered expression in a tumor cell or cancerous tissue in comparison to a normal cell or tissue.

As used herein the term “antigen-based vaccine” is a vaccine composition based on one or more antigens, e.g., a plurality of antigens. The vaccines can be nucleotide-based (e.g., virally based, RNA based, or DNA based), protein-based (e.g., peptide based), or a combination thereof.

As used herein the term “candidate antigen” is a mutation or other aberration giving rise to a sequence that may represent an antigen.

As used herein the term “coding region” is the portion(s) of a gene that encode protein.

As used herein the term “coding mutation” is a mutation occurring in a coding region.

As used herein the term “ORF” means open reading frame.

As used herein the term “NEO-ORF” is a tumor-specific ORF arising from a mutation or other aberration such as splicing.

As used herein the term “missense mutation” is a mutation causing a substitution from one amino acid to another.

As used herein the term “nonsense mutation” is a mutation causing a substitution from an amino acid to a stop codon or causing removal of a canonical start codon.

As used herein the term “frameshift mutation” is a mutation causing a change in the frame of the protein.

As used herein the term “indel” is an insertion or deletion of one or more nucleic acids.

As used herein, the term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Alternatively, sequence similarity or dissimilarity can be established by the combined presence or absence of particular nucleotides, or, for translated sequences, amino acids at selected sequence positions (e.g., sequence motifs).

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information.

As used herein the term “non-stop or read-through” is a mutation causing the removal of the natural stop codon.

As used herein the term “epitope” is the specific portion of an antigen typically bound by an antibody or T cell receptor.

As used herein the term “immunogenic” is the ability to elicit an immune response, e.g., via T cells, B cells, or both.

As used herein the term “HLA binding affinity” “MHC binding affinity” means affinity of binding between a specific antigen and a specific MHC allele.

As used herein the term “bait” is a nucleic acid probe used to enrich a specific sequence of DNA or RNA from a sample.

As used herein the term “variant” is a difference between a subject's nucleic acids and the reference human genome used as a control.

As used herein the term “variant call” is an algorithmic determination of the presence of a variant, typically from sequencing.

As used herein the term “polymorphism” is a germline variant, i.e., a variant found in all DNA-bearing cells of an individual.

As used herein the term “somatic variant” is a variant arising in non-germline cells of an individual.

As used herein the term “allele” is a version of a gene or a version of a genetic sequence or a version of a protein.

As used herein the term “HLA type” is the complement of HLA gene alleles.

As used herein the term “nonsense-mediated decay” or “NMD” is a degradation of an mRNA by a cell due to a premature stop codon.

As used herein the term “truncal mutation” is a mutation originating early in the development of a tumor and present in a substantial portion of the tumor's cells.

As used herein the term “subclonal mutation” is a mutation originating later in the development of a tumor and present in only a subset of the tumor's cells.

As used herein the term “exome” is a subset of the genome that codes for proteins. An exome can be the collective exons of a genome.

As used herein the term “logistic regression” is a regression model for binary data from statistics where the logit of the probability that the dependent variable is equal to one is modeled as a linear function of the dependent variables.

As used herein the term “neural network” is a machine learning model for classification or regression consisting of multiple layers of linear transformations followed by element-wise nonlinearities typically trained via stochastic gradient descent and back-propagation.

As used herein the term “proteome” is the set of all proteins expressed and/or translated by a cell, group of cells, or individual.

As used herein the term “peptidome” is the set of all peptides presented by MHC-I or MHC-II on the cell surface. The peptidome may refer to a property of a cell or a collection of cells (e.g., the tumor peptidome, meaning the union of the peptidomes of all cells that comprise the tumor).

As used herein the term “ELISPOT” means Enzyme-linked immunosorbent spot assay—which is a common method for monitoring immune responses in humans and animals.

As used herein the term “dextramers” is a dextran-based peptide-MHC multimers used for antigen-specific T-cell staining in flow cytometry.

As used herein the term “tolerance or immune tolerance” is a state of immune non-responsiveness to one or more antigens, e.g. self-antigens.

As used herein the term “central tolerance” is a tolerance affected in the thymus, either by deleting self-reactive T-cell clones or by promoting self-reactive T-cell clones to differentiate into immunosuppressive regulatory T-cells (Tregs).

As used herein the term “peripheral tolerance” is a tolerance affected in the periphery by downregulating or anergizing self-reactive T-cells that survive central tolerance or promoting these T cells to differentiate into Tregs.

The term “sample” can include a single cell or multiple cells or fragments of cells or an aliquot of body fluid, taken from a subject, by means including venipuncture, excretion, ejaculation, massage, biopsy, needle aspirate, lavage sample, scraping, surgical incision, or intervention or other means known in the art.

The term “subject” encompasses a cell, tissue, or organism, human or non-human, whether in vivo, ex vivo, or in vitro, male or female. The term subject is inclusive of mammals including humans.

The term “mammal” encompasses both humans and non-humans and includes but is not limited to humans, non-human primates, canines, felines, murines, bovines, equines, and porcines.

The term “clinical factor” refers to a measure of a condition of a subject, e.g., disease activity or severity. “Clinical factor” encompasses all markers of a subject's health status, including non-sample markers, and/or other characteristics of a subject, such as, without limitation, age and gender. A clinical factor can be a score, a value, or a set of values that can be obtained from evaluation of a sample (or population of samples) from a subject or a subject under a determined condition. A clinical factor can also be predicted by markers and/or other parameters such as gene expression surrogates. Clinical factors can include tumor type, tumor sub-type, and smoking history.

The term “antigen-encoding nucleic acid sequences derived from a tumor” refers to nucleic acid sequences directly extracted from the tumor, e.g. via RT-PCR; or sequence data obtained by sequencing the tumor and then synthesizing the nucleic acid sequences using the sequencing data, e.g., via various synthetic or PCR-based methods known in the art.

The term “antigen-encoding nucleic acid sequences derived from an infection” refers to nucleic acid sequences directly extracted from infected cells or a infectious disease organism, e.g. via RT-PCR; or sequence data obtained by sequencing the infected cell or infectious disease organism and then synthesizing the nucleic acid sequences using the sequencing data, e.g., via various synthetic or PCR-based methods known in the art.

The term “alphavirus” refers to members of the family Togaviridae, and are positive-sense single-stranded RNA viruses. Alphaviruses are typically classified as either Old World, such as Sindbis, Ross River, Mayaro, Chikungunya, and Semliki Forest viruses, or New World, such as eastern equine encephalitis, Aura, Fort Morgan, or Venezuelan equine encephalitis and its derivative strain TC-83. Alphaviruses are typically self-replicating RNA viruses.

The term “alphavirus backbone” refers to minimal sequence(s) of an alphavirus that allow for self-replication of the viral genome. Minimal sequences can include conserved sequences for nonstructural protein-mediated amplification, a nonstructural protein 1 (nsP1) gene, a nsP2 gene, a nsP3 gene, a nsP4 gene, and a polyA sequence, as well as sequences for expression of subgenomic viral RNA including a 26S promoter element.

The term “sequences for nonstructural protein-mediated amplification” includes alphavirus conserved sequence elements (CSE) well known to those in the art. CSEs include, but are not limited to, an alphavirus 5′ UTR, a 51-nt CSE, a 24-nt CSE, or other 26S subgenomic promoter sequence, a 19-nt CSE, and an alphavirus 3′ UTR.

The term “RNA polymerase” includes polymerases that catalyze the production of RNA polynucleotides from a DNA template. RNA polymerases include, but are not limited to, bacteriophage derived polymerases including T3, T7, and SP6.

The term “lipid” includes hydrophobic and/or amphiphilic molecules. Lipids can be cationic, anionic, or neutral. Lipids can be synthetic or naturally derived, and in some instances biodegradable. Lipids can include cholesterol, phospholipids, lipid conjugates including, but not limited to, polyethyleneglycol (PEG) conjugates (PEGylated lipids), waxes, oils, glycerides, fats, and fat-soluble vitamins. Lipids can also include dilinoleylmethyl-4-dimethylaminobutyrate (MC3) and MC3-like molecules.

The term “lipid nanoparticle” or “LNP” includes vesicle like structures formed using a lipid containing membrane surrounding an aqueous interior, also referred to as liposomes. Lipid nanoparticles includes lipid-based compositions with a solid lipid core stabilized by a surfactant. The core lipids can be fatty acids, acylglycerols, waxes, and mixtures of these surfactants. Biological membrane lipids such as phospholipids, sphingomyelins, bile salts (sodium taurocholate), and sterols (cholesterol) can be utilized as stabilizers. Lipid nanoparticles can be formed using defined ratios of different lipid molecules, including, but not limited to, defined ratios of one or more cationic, anionic, or neutral lipids. Lipid nanoparticles can encapsulate molecules within an outer-membrane shell and subsequently can be contacted with target cells to deliver the encapsulated molecules to the host cell cytosol. Lipid nanoparticles can be modified or functionalized with non-lipid molecules, including on their surface. Lipid nanoparticles can be single-layered (unilamellar) or multi-layered (multilamellar). Lipid nanoparticles can be complexed with nucleic acid. Unilamellar lipid nanoparticles can be complexed with nucleic acid, wherein the nucleic acid is in the aqueous interior. Multilamellar lipid nanoparticles can be complexed with nucleic acid, wherein the nucleic acid is in the aqueous interior, or to form or sandwiched between

Abbreviations: MHC: major histocompatibility complex; HLA: human leukocyte antigen, or the human MHC gene locus; NGS: next-generation sequencing; PPV: positive predictive value; TSNA: tumor-specific neoantigen; FFPE: formalin-fixed, paraffin-embedded; NMD: nonsense-mediated decay; NSCLC: non-small-cell lung cancer; DC: dendritic cell.

It should be noted that, as used in the specification and the appended claims, the singular forms “a,” “an,” and “the” include plural referents unless the context clearly dictates otherwise.

Unless specifically stated or otherwise apparent from context, as used herein the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any terms not directly defined herein shall be understood to have the meanings commonly associated with them as understood within the art of the invention. Certain terms are discussed herein to provide additional guidance to the practitioner in describing the compositions, devices, methods and the like of aspects of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the invention herein.

All references, issued patents and patent applications cited within the body of the specification are hereby incorporated by reference in their entirety, for all purposes.

### II. Antigen Identification

Research methods for NGS analysis of tumor and normal exome and transcriptomes have been described and applied in the antigen identification space.^{6,14,15} Certain optimizations for greater sensitivity and specificity for antigen identification in the clinical setting can be considered. These optimizations can be grouped into two areas, those related to laboratory processes and those related to the NGS data analysis. The research methods described can also be applied to identification of antigens in other settings, such as identification of identifying antigens from an infectious disease organism, an infection in a subject, or an infected cell of a subject. Examples of optimizations are known to those skilled in the art, for example the methods described in more detail in U.S. Pat. No. 10,055,540, US Application Pub. No. US20200010849A1, and international patent application publications WO/2018/195357 and WO/2018/208856, each herein incorporated by reference, in their entirety, for all purposes.

Methods for identifying antigens (e.g., antigens derived from a tumor or an infectious disease organism) include identifying antigens that are likely to be presented on a cell surface (e.g., presented by MHC on a tumor cell, an infected cell, or an immune cell, including professional antigen presenting cells such as dendritic cells), and/or are likely to be immunogenic. As an example, one such method may comprise the steps of: obtaining at least one of exome, transcriptome or whole genome nucleotide sequencing and/or expression data from a tumor, an infected cell, or an infectious disease organism, wherein the nucleotide sequencing data and/or expression data is used to obtain data representing peptide sequences of each of a set of antigens (e.g., antigens derived from the tumor or infectious disease organism); inputting the peptide sequence of each antigen into one or more presentation models to generate a set of numerical likelihoods that each of the antigens is presented by one or more MHC alleles on a cell surface, such as a tumor cell or an infected cell of the subject, the set of numerical likelihoods having been identified at least based on received mass spectrometry data; and selecting a subset of the set of antigens based on the set of numerical likelihoods to generate a set of selected antigens.

### III. Identification of Tumor Specific Mutations in Neoantigens

Also disclosed herein are methods for the identification of certain mutations (e.g., the variants or alleles that are present in cancer cells). In particular, these mutations can be present in the genome, transcriptome, proteome, or exome of cancer cells of a subject having cancer but not in normal tissue from the subject. Specific methods for identifying neoantigens, including shared neoantigens, that are specific to tumors are known to those skilled in the art, for example the methods described in more detail in U.S. Pat. No. 10,055,540, US Application Pub. No. US20200010849A1, and international patent application publications WO/2018/195357 and WO/2018/208856, each herein incorporated by reference, in their entirety, for all purposes. Examples of shared neoantigens that are specific to tumors are described in more detail in international patent application publication WO2019226941A1, herein incorporated by reference in its entirety, for all purposes.

Shared antigens include, but are not limited to shared Cancer Testis Antigens (CTAs), including but not limited to MAGEA1, MAGEA3, MAGEA4, MAGEA6, MAGEA8, MAGEA11, MAGEB2, CTCFL, and/or CT83. CTA-associated MHC class I epitopes include, but are not limited to, FVQENYLEY, EVDPTSHSY, NTDNNLAVY, EVDPIGHLY, GVYDGREHTV, ALREEGEGV, KVLEYVIKV, GVYDGEEHSV, KLVELEHTL, AETSYVKVL, KVLEHVVRV, EADPTGHSY, SALPTTISF, GVYDGREHTVY, TQHFVQENY, EYVIKVSAR, LVRPSSSGL, GEMSSNSTAL, TVYGEPRKL, ALAETSYVK, TSYVKVLEH, YPSLREAAL, ALLEEEEGV, GPRQSLQQC, IAYPSLREAAL, and/or MEVDPIGHL. Shared antigens can include those described in U.S. Application publication US20210196806A1, such as those referred to by SEQ ID NOS. 57-10,754 that include shared antigens associated with gene expressed at a level of at least 10 TPM in at least 0.98% of cancer cases, which is herein incorporated by references for all purposes.

Genetic mutations in tumors can be considered useful for the immunological targeting of tumors if they lead to changes in the amino acid sequence of a protein exclusively in the tumor. Useful mutations include: (1) non-synonymous mutations leading to different amino acids in the protein; (2) read-through mutations in which a stop codon is modified or deleted, leading to translation of a longer protein with a novel tumor-specific sequence at the C-terminus; (3) splice site mutations that lead to the inclusion of an intron in the mature mRNA and thus a unique tumor-specific protein sequence; (4) chromosomal rearrangements that give rise to a chimeric protein with tumor-specific sequences at the junction of 2 proteins (i.e., gene fusion); (5) frameshift mutations or deletions that lead to a new open reading frame with a novel tumor-specific protein sequence. Mutations can also include one or more of nonframeshift indel, missense or nonsense substitution, splice site alteration, genomic rearrangement or gene fusion, or any genomic or expression alteration giving rise to a neoORF.

Peptides with mutations or mutated polypeptides arising from for example, splice-site, frameshift, readthrough, or gene fusion mutations in tumor cells can be identified by sequencing DNA, RNA or protein in tumor versus normal cells.

Also mutations can include previously identified tumor specific mutations. Known tumor mutations can be found at the Catalogue of Somatic Mutations in Cancer (COSMIC) database.

A variety of methods are available for detecting the presence of a particular mutation or allele in an individual's DNA or RNA. Advancements in this field have provided accurate, easy, and inexpensive large-scale SNP genotyping. For example, several techniques have been described including dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. These methods utilize amplification of a target genetic region, typically by PCR. Still other methods, based on the generation of small signal molecules by invasive cleavage followed by mass spectrometry or immobilized padlock probes and rolling-circle amplification. Several of the methods known in the art for detecting specific mutations are summarized below.

PCR based detection means can include multiplex amplification of a plurality of markers simultaneously. For example, it is well known in the art to select PCR primers to generate PCR products that do not overlap in size and can be analyzed simultaneously. Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

Several methods have been developed to facilitate analysis of single nucleotide polymorphisms in genomic DNA or cellular RNA. For example, a single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide(s) present in the polymorphic site of the target molecule is complementary to that of the nucleotide derivative used in the reaction. This method has the advantage that it does not require the determination of large amounts of extraneous sequence data.

A solution-based method can be used for determining the identity of a nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. can be a heterogeneous phase assay, in which the primer or the target molecule is immobilized to a solid phase.

Several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA in that they utilize incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J. Hum. Genet. 52:46-59 (1993)).

A number of initiatives obtain sequence information directly from millions of individual molecules of DNA or RNA in parallel. Real-time single molecule sequencing-by-synthesis technologies rely on the detection of fluorescent nucleotides as they are incorporated into a nascent strand of DNA that is complementary to the template being sequenced. In one method, oligonucleotides 30-50 bases in length are covalently anchored at the 5′ end to glass cover slips. These anchored strands perform two functions. First, they act as capture sites for the target template strands if the templates are configured with capture tails complementary to the surface-bound oligonucleotides. They also act as primers for the template directed primer extension that forms the basis of the sequence reading. The capture primers function as a fixed position site for sequence determination using multiple cycles of synthesis, detection, and chemical cleavage of the dye-linker to remove the dye. Each cycle includes adding the polymerase/labeled nucleotide mixture, rinsing, imaging and cleavage of dye. In an alternative method, polymerase is modified with a fluorescent donor molecule and immobilized on a glass slide, while each nucleotide is color-coded with an acceptor fluorescent moiety attached to a gamma-phosphate. The system detects the interaction between a fluorescently-tagged polymerase and a fluorescently modified nucleotide as the nucleotide becomes incorporated into the de novo chain. Other sequencing-by-synthesis technologies also exist.

Any suitable sequencing-by-synthesis platform can be used to identify mutations. As described above, four major sequencing-by-synthesis platforms are currently available: the Genome Sequencers from Roche/454 Life Sciences, the 1 G Analyzer from Illumina/Solexa, the SOLiD system from Applied BioSystems, and the Heliscope system from Helicos Biosciences. Sequencing-by-synthesis platforms have also been described by Pacific BioSciences and VisiGen Biotechnologies. In some embodiments, a plurality of nucleic acid molecules being sequenced is bound to a support (e.g., solid support). To immobilize the nucleic acid on a support, a capture sequence/universal priming site can be added at the 3′ and/or 5′ end of the template. The nucleic acids can be bound to the support by hybridizing the capture sequence to a complementary sequence covalently attached to the support. The capture sequence (also referred to as a universal capture sequence) is a nucleic acid sequence complementary to a sequence attached to a support that may dually serve as a universal primer.

As an alternative to a capture sequence, a member of a coupling pair (such as, e.g., antibody/antigen, receptor/ligand, or the avidin-biotin pair as described in, e.g., US Patent Application No. 2006/0252077) can be linked to each fragment to be captured on a surface coated with a respective second member of that coupling pair.

Subsequent to the capture, the sequence can be analyzed, for example, by single molecule detection/sequencing, e.g., as described in the Examples and in U.S. Pat. No. 7,283,337, including template-dependent sequencing-by-synthesis. In sequencing-by-synthesis, the surface-bound molecule is exposed to a plurality of labeled nucleotide triphosphates in the presence of polymerase. The sequence of the template is determined by the order of labeled nucleotides incorporated into the 3′ end of the growing chain. This can be done in real time or can be done in a step-and-repeat mode. For real-time analysis, different optical labels to each nucleotide can be incorporated and multiple lasers can be utilized for stimulation of incorporated nucleotides.

Sequencing can also include other massively parallel sequencing or next generation sequencing (NGS) techniques and platforms. Additional examples of massively parallel sequencing techniques and platforms are the Illumina HiSeq or MiSeq, Thermo PGM or Proton, the Pac Bio RS II or Sequel, Qiagen's Gene Reader, and the Oxford Nanopore MinION. Additional similar current massively parallel sequencing technologies can be used, as well as future generations of these technologies.

Any cell type or tissue can be utilized to obtain nucleic acid samples for use in methods described herein. For example, a DNA or RNA sample can be obtained from a tumor or a bodily fluid, e.g., blood, obtained by known techniques (e.g. venipuncture) or saliva. Alternatively, nucleic acid tests can be performed on dry samples (e.g. hair or skin). In addition, a sample can be obtained for sequencing from a tumor and another sample can be obtained from normal tissue for sequencing where the normal tissue is of the same tissue type as the tumor. A sample can be obtained for sequencing from a tumor and another sample can be obtained from normal tissue for sequencing where the normal tissue is of a distinct tissue type relative to the tumor.

Tumors can include one or more of lung cancer, melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, and T cell lymphocytic leukemia, non-small cell lung cancer, and small cell lung cancer.

Alternatively, protein mass spectrometry can be used to identify or validate the presence of mutated peptides bound to MHC proteins on tumor cells. Peptides can be acid-eluted from tumor cells or from HLA molecules that are immunoprecipitated from tumor, and then identified using mass spectrometry.

### IV. Antigens

Antigens can include nucleotides or polypeptides. For example, an antigen can be an RNA sequence that encodes for a polypeptide sequence. Antigens useful in vaccines can therefore include nucleotide sequences or polypeptide sequences.

Disclosed herein are peptides derived from any polypeptide known to or have been found to have altered expression in a tumor cell or cancerous tissue in comparison to a normal cell or tissue, for example any polypeptide known to or have been found to be aberrantly expressed in a tumor cell or cancerous tissue in comparison to a normal cell or tissue. Suitable polypeptides from which the antigenic peptides can be derived can be found for example in the COSMIC database. Shared antigens with altered expression include, but are not limited to, Cancer Testis Antigens (CTA)-associated epitopes (e.g., a MAGEA1 MHC class I epitope, a MAGEA3 MHC class I epitope, a MAGEA4 MHC class I epitope, a MAGEA6 MHC class I epitope, a MAGEA11 MHC class I epitope, a MAGEB2 MHC class I epitope, a CTCFL MHC class I epitope, and/or a CT83 MHC class I epitope). Shared antigens can include those described in U.S. Application publication US20210196806A1, such as those referred to by SEQ ID NOS. 57-10,754 that include shared antigens associated with gene expressed at a level of at least 10 TPM in at least 0.98% of cancer cases, which is herein incorporated by references for all purposes.

Disclosed herein are vaccine systems that can encode a CTA-associated epitope and combinations thereof. A CTA-associated epitope can be a MAGEA1 MHC class I epitope, a MAGEA3 MHC class I epitope, a MAGEA4 MHC class I epitope, a MAGEA6 MHC class I epitope, a MAGEA11 MHC class I epitope, a MAGEB2 MHC class I epitope, and/or a CT83 MHC class I epitope. A vaccine system can encode a combination of CTA-associated epitopes that can include a MAGEA1 MHC class I epitope, a MAGEA3 MHC class I epitope, a MAGEA4 MHC class I epitope, a MAGEA6 MHC class I epitope, a MAGEA11 MHC class I epitope, a MAGEB2 MHC class I epitope, a CTCFL MHC class I epitope, and/or a CT83 MHC class I epitope. Exemplary epitopes are shown in Table 2A.

A CTA-associated epitope can be a MAGEA1 MHC class I epitope. A CTA-associated epitope can be a MAGEA3 MHC class I epitope. A CTA-associated epitope can be a MAGEA4 MHC class I epitope. A CTA-associated epitope can be a MAGEA6 MHC class I epitope. A CTA-associated epitope can be a MAGEA11 MHC class I epitope. A CTA-associated epitope can be a MAGEB2 MHC class I epitope. A CTA-associated epitope can be and/or a CT83 MHC class I epitope. A vaccine system can encode a combination of CTA-associated epitopes that can include a MAGEA1 MHC class I epitope. A CTA-associated epitope can be a MAGEA3 MHC class I epitope. A CTA-associated epitope can be a MAGEA4 MHC class I epitope. A CTA-associated epitope can be a MAGEA6 MHC class I epitope. A CTA-associated epitope can be a MAGEA11 MHC class I epitope. A CTA-associated epitope can be a MAGEB2 MHC class I epitope. A CTA-associated epitope can be a CTCFL MHC class I epitope. A CTA-associated epitope can be a CT83 MHC class I epitope.

A vaccine system can include a MAGEA1 MHC class I epitope encoding nucleic acid sequence, a MAGEA3 MHC class I epitope encoding nucleic acid sequence, a MAGEA4 MHC class I epitope encoding nucleic acid sequence, a MAGEA6 MHC class I epitope encoding nucleic acid sequence, a MAGEA8 MHC class I epitope encoding nucleic acid sequence, a MAGEA11 MHC class I epitope encoding nucleic acid sequence, a MAGEB2 MHC class I epitope encoding nucleic acid sequence, a CTCFL MHC class I epitope encoding nucleic acid sequence, a CT83 MHC class I epitope encoding nucleic acid sequence, and combinations thereof.

A vaccine system can include a MAGEA1 MHC class I epitope encoding nucleic acid sequence. A vaccine system can include a MAGEA3 MHC class I epitope encoding nucleic acid sequence. A vaccine system can include a MAGEA4 MHC class I epitope encoding nucleic acid sequence. A vaccine system can include a MAGEA6 MHC class I epitope encoding nucleic acid sequence. A vaccine system can include a MAGEA8 MHC class I epitope encoding nucleic acid sequence. A vaccine system can include a MAGEA11 MHC class I epitope encoding nucleic acid sequence. A vaccine system can include a MAGEB2 MHC class I epitope encoding nucleic acid sequence. A vaccine system can include a CTCFL MHC class I epitope encoding nucleic acid sequence a CT83 MHC class I epitope encoding nucleic acid sequence.

A CTA-associated epitope can include, but are not limited to, the following MHC class I epitopes: FVQENYLEY, EVDPTSHSY, NTDNNLAVY, EVDPIGHLY, GVYDGREHTV, ALREEGEGV, KVLEYVIKV, GVYDGEEHSV, KLVELEHTL, AETSYVKVL, KVLEHVVRV, EADPTGHSY, SALPTTISF, GVYDGREHTVY, TQHFVQENY, EYVIKVSAR, LVRPSSSGL, GEMSSNSTAL, TVYGEPRKL, ALAETSYVK, TSYVKVLEH, YPSLREAAL, ALLEEEEGV, GPRQSLQQC, IAYPSLREAAL, or MEVDPIGHL.

A vaccine system can include defined combinations of CTA-encoding nucleic acid sequences. A vaccine system can include each of a MAGEA1 MHC class I epitope encoding nucleic acid sequence, a MAGEA4 MHC class I epitope encoding nucleic acid sequence, a MAGEB2 MHC class I epitope encoding nucleic acid sequence, a MAGEA11 MHC class I epitope encoding nucleic acid sequence, a MAGEA3 MHC class I epitope encoding nucleic acid sequence, and a CT83 MHC class I epitope encoding nucleic acid sequence. A vaccine system can include each of a MAGEA6 MHC class I epitope encoding nucleic acid sequence, a MAGEA11 MHC class I epitope encoding nucleic acid sequence, a MAGEA3 MHC class I epitope encoding nucleic acid sequence, and a CT83 MHC class I epitope encoding nucleic acid sequence. A vaccine system can include each of a MAGEA1 MHC class I epitope encoding nucleic acid sequence, a MAGEA4 MHC class I epitope encoding nucleic acid sequence, a MAGEB2 MHC class I epitope encoding nucleic acid sequence, a MAGEA11 MHC class I epitope encoding nucleic acid sequence, and a CT83 MHC class I epitope encoding nucleic acid sequence. A vaccine system can include each of a MAGEA3 MHC class I epitope encoding nucleic acid sequence, a MAGEA4 MHC class I epitope encoding nucleic acid sequence, a MAGEB2 MHC class I epitope encoding nucleic acid sequence, a MAGEA11 MHC class I epitope encoding nucleic acid sequence, and a CT83 MHC class I epitope encoding nucleic acid sequence.

A vaccine system can include each of a CT83 MHC class I epitope encoding nucleic acid sequence, a MAGEA4 MHC class I epitope encoding nucleic acid sequence, a MAGEA1 MHC class I epitope encoding nucleic acid sequence, a MAGEA8, a MAGEB2 MHC class I epitope encoding nucleic acid sequence, a MAGEA3 MHC class I epitope encoding nucleic acid sequence, and a MAGEA11 MHC class I epitope encoding nucleic acid sequence. In such a system, each of the encoded CTA-associated MHC class I epitopes can include NTDNNLAVY, LVRPSSSGL, GEMSSNSTAL, GVYDGREHTV, GVYDGREHTVY, ALAETSYVK, KVLEHVVRV, SALPTTISF, GVYDGEEHSV, KVLEYVIKV, AETSYVKVL, EYVIKVSAR, EVDPIGHLY, MEVDPIGHL, and EVDPTSHSY. In such a system, each of the encoded CTA-associated MHC class I epitopes can be encoded as a single polypeptide, such as the amino acid sequence

ELGVMGVYDGREHTVYGEPRKELGVMGVYDGREHTVYGEPRKGID
VKEVDPTSHSYVLVTSGPRALAETSYVKVLEHVVRVNARVRGPRA
LAETSYVKVLEYVIKVSARVRFFFFLNMLGVYDGEEHSVFGEPWF
QRNTGEMSSNSTALALVRPSSSGLINSNTDNNLAVYDLSRDSPQG
ASALPTTISFTCWRQGIELMEVDPIGHLYIFATCFQRNTGEMSSN
STALALVRPSSSGLINSNTDNNLAVYDLSRDSPQGASALPTTISF
TCWRQGIELMEVDPIGHLYIFATCGPRALAETSYVKVLEHVVRVN
ARVRGPRALAETSYVKVLEYVIKVSARVRFFFFLNMLGVYDGEEH
SVFGEPWGIDVKEVDPTSHSYVLVTS.

A vaccine system can include each of a CT83 MHC class I epitope encoding nucleic acid sequence, a MAGEA4 MHC class I epitope encoding nucleic acid sequence, a MAGEA1 MHC class I epitope encoding nucleic acid sequence, a MAGEA8, a MAGEA6 MHC class I epitope encoding nucleic acid sequence, a CTCFL MHC class I epitope encoding nucleic acid sequence, and a MAGEA3 MHC class I epitope encoding nucleic acid sequence. In such a system, each of the encoded CTA-associated MHC class I epitopes can include NTDNNLAVY, LVRPSSSGL, GEMSSNSTAL, KLVELEHTL, GVYDGREHTV, GVYDGREHTVY, ALAETSYVK, KVLEHVVRV, ALLEEEEGV, YPSLREAAL, IAYPSLREAAL, AETSYVKVL, FVQENYLEY, EVDPIGHLY, MEVDPIGHL, and GPRQSLQQC. In such a system, each of the encoded CTA-associated MHC class I epitopes can be encoded as a single polypeptide, such as the amino acid sequence

GPRALAETSYVKVLEHVVRVNARVRIAYPSLREAALLEEEEGVWL
EEGPRQSLQQCVAISLLTQYFVQENYLEYRQVPGMVENKLVELEH
TLLSKGIELMEVDPIGHLYIFATCQRNTGEMSSNSTALALVRPSS
SGLINSNTDNNLAVYDLSRWEELGVMGVYDGREHTVYGEPRKLLT
QDQRNTGEMSSNSTALALVRPSSSGLINSNTDNNLAVYDLSRWEE
LGVMGVYDGREHTVYGEPRKLLTQDWMVENKLVELEHTLLSKGIE
LMEVDPIGHLYIFATCPRALAETSYVKVLEHVVRVNARVRIAYPS
LREAALLEEEEGVWLEEGPRQSLQQCVAISLLTQYFVQENYLEYR
QVPG.

CTA-associated MHC class I epitopes can include native N- and/or C-terminal flanking sequences of the therapeutic vaccine epitope in the context of the native CTA protein. CTA-associated MHC class I epitopes that include native flanking sequences can be linked (concatenated) to other epitopes encoded in a cassette, including other epitopes (e.g., other CTA-associated MHC class I epitopes and/or KRAS-associated MHC class I neoepitopes) that include their respective native flanking sequences. CTA-associated MHC class I epitopes can concatenated to other epitopes encoded in a cassette, including interspersed with non-CTA-associated epitopes, e.g., interspersed with one or more KRAS-associated MHC class I neoepitopes.

Disclosed herein are vaccine systems that can encode KRAS-associated MHC class I neoepitopes. KRAS-associated MHC class I neoepitopes include, but are not limited to, neoepitopes having KRAS G12 mutations and/or KRAS Q61 mutations. A vaccine system can include iterations of KRAS-associated MHC class I neoepitopes having a KRAS G12 mutation. A vaccine system can include iterations of KRAS-associated MHC class I neoepitopes having a KRAS Q61 mutation. A vaccine system can include iterations of KRAS-associated MHC class I neoepitopes having KRAS G12C, KRAS G12V, KRAS G12D, and/or KRAS Q61H mutations. A vaccine system can include iterations of KRAS-associated MHC class I neoepitopes having a KRAS G12C mutation. A vaccine system can include iterations of KRAS-associated MHC class I neoepitopes having a KRAS G12V mutation. A vaccine system can include iterations of KRAS-associated MHC class I neoepitopes having a KRAS G12D mutation. A vaccine system can include iterations of KRAS-associated MHC class I neoepitopes having a KRAS Q61H mutation. A vaccine system can include iterations of each of KRAS-associated MHC class I neoepitopes having a KRAS G12C, KRAS G12V, KRAS G12D, and KRAS Q61H mutation. A vaccine system can include iterations of at least two distinct KRAS-associated MHC class I neoepitopes selected from the group consisting of: a KRAS G12C, KRAS G12V, KRAS G12D, and KRAS Q61H mutation. A vaccine system can include iterations of at least three distinct KRAS-associated MHC class I neoepitopes selected from the group consisting of: a KRAS G12C, KRAS G12V, KRAS G12D, and KRAS Q61H mutation. A vaccine system can include iterations only of a single distinct KRAS-associated MHC class I neoepitope. A vaccine system can include iterations only of a single distinct KRAS-associated MHC class I neoepitope having a KRAS G12C mutation. A vaccine system can include iterations only of a single distinct KRAS-associated MHC class I neoepitope having a KRAS G12D mutation. A vaccine system can include iterations only of a single distinct KRAS-associated MHC class I neoepitope having a KRAS G12V mutation. A vaccine system can include iterations only of a single distinct KRAS-associated MHC class I neoepitope having a KRAS Q61H mutation.

KRAS-associated MHC class I neoepitopes having a KRAS G12C mutation include VVVGACGVGK or KLVVVGACGV. KRAS-associated MHC class I neoepitopes having a KRAS G12D mutation include VVGADGVGK or VVVGADGVGK, KRAS-associated MHC class I neoepitopes having a KRAS G12V mutation include VVGAVGVGK, VVVGAVGVGK, or AVGVGKSAL.

A vaccine system can include iterations of each of KRAS-associated MHC class I neoepitopes having the amino acid sequences VVVGACGVGK, VVVGADGVGK, VVGAVGVGK, and ILDTAGHEEY. A vaccine system can include iterations of at least two distinct KRAS-associated MHC class I neoepitopes having the amino acid sequences selected from the group consisting of: VVVGACGVGK, VVVGADGVGK, VVGAVGVGK, and ILDTAGHEEY. A vaccine system can include iterations of at least three distinct KRAS-associated MHC class I neoepitopes having the amino acid sequences selected from the group consisting of: VVVGACGVGK, VVVGADGVGK, VVGAVGVGK, and ILDTAGHEEY. A vaccine system can include iterations of at least one of KRAS-associated MHC class I neoepitopes having the amino acid sequences VVVGACGVGK, VVVGADGVGK, VVGAVGVGK, and ILDTAGHEEY.

KRAS-associated MHC class I neoepitopes can include native N- and/or C-terminal flanking sequences of the therapeutic vaccine epitope in the context of the native KRAS protein. Illustrative non-limiting examples of KRAS-associated MHC class I neoepitopes are the 25mers MTEYKLVVVGACGVGKSALTIQLIQ for KRAS G12C, MTEYKLVVVGADGVGKSALTIQLIQ for KRAS G12D, MTEYKLVVVGAVGVGKSALTIQLIQ for KRAS G12V, and ETCLLDILDTAGHEEYSAMRDQYMR for KRAS Q61H. KRAS-associated MHC class I neoepitopes that include native flanking sequences can be linked (concatenated) to other (neo)epitopes encoded in a cassette, including other (neo)epitopes (e.g., other KRAS-associated MHC class I neoepitopes) that include their respective native flanking sequences. An illustrative non-limiting cassette includes concantenated KRAS-associated MHC class I neoepitopes that are linked through their native flanking sequences and that includes 4 iterations for each of the KRAS neoepitopes having the mutations KRAS G12C, KRAS G12D, KRAS G12V, and KRAS Q61H. Another illustrative non-limiting cassette of concantenated KRAS-associated MHC class I neoepitopes that are linked through their native flanking sequences and that includes 4 iterations for each of the KRAS neoepitopes having the mutations KRAS G12C and KRAS G12V.

Epitope-encoding nucleic acid sequences that encode KRAS-associated MHC class I neoepitopes, such as those that include native N- and/or C-terminal flanking sequences, can encode multiple known and/or predicted KRAS-associated MHC class I neoepitopes. As an illustrative example, the KRAS G12V 25mer MTEYKLVVVGAVGVGKSALTIQLIQ encodes each of the known and/or predicted KRAS-associated MHC class I neoepitopes, VVVGAVGVGK, and AVGVGKSAL.

Epitope-encoding nucleic acid sequences, including those that encode KRAS-associated MHC class I neoepitopes, can be in any order in a cassette. Epitope-encoding nucleic acid sequences, including those that encode KRAS-associated MHC class I neoepitopes, can be in an order that minimizes junctional epitopes, as described further herein. As an illustrative non-limiting example, concantenated KRAS-associated MHC class I neoepitopes linked together to minimize junctional epitopes can have the order: G12C G12D Q61H G12D G12V G12C Q61H G12D G12V G12C Q61H G12D G12V Q61H G12V G12C, such as represented by the amino acid sequence MTEYKLVVVGACGVGKSALTIQLIQMTEYKLVVVGADGVGKSALTIQLIQETCLLDILDTA GHEEYSAMRDQYMRMTEYKLVVVGADGVGKSALTIQLIQMTEYKLVVVGAVGVGKSAL TIQLIQMTEYKLVVVGACGVGKSALTIQLIQETCLLDILDTAGHEEYSAMRDQYMRMTEY KLVVVGADGVGKSALTIQLIQMTEYKLVVVGAVGVGKSALTIQLIQMTEYKLVVVGACG VGKSALTIQLIQETCLLDILDTAGHEEYSAMRDQYMRMTEYKLVVVGADGVGKSALTIQLI QMTEYKLVVVGAVGVGKSALTIQLIQETCLLDILDTAGHEEYSAMRDQYMRMTEYKLVV VGAVGVGKSALTIQLIQMTEYKLVVVGACGVGKSALTIQLIQ. Another representative amino acid sequence that includes concatenated KRAS-associated MHC class I neoepitopes that include native N- and/or C-terminal flanking sequences is represented by the amino acid sequence

MTEYKLVVVGAVGVGKSALTIQLIQMTEYKLVVVGAVGVGKSALT
IQLIQMTEYKLVVVGAVGVGKSALTIQLIQMTEYKLVVVGAVGVG
KSALTIQLIQMTEYKLVVVGACGVGKSALTIQLIQMTEYKLVVVG
ACGVGKSALTIQLIQMTEYKLVVVGACGVGKSALTIQLIQMTEYK
LVVVGACGVGKSALTIQLIQ.

Also disclosed herein are isolated peptides that comprise tumor specific mutations identified by the methods disclosed herein, peptides that comprise known tumor specific mutations, and mutant polypeptides or fragments thereof identified by methods disclosed herein. Neoantigen peptides can be described in the context of their coding sequence where a neoantigen includes the nucleotide sequence (e.g., DNA or RNA) that codes for the related polypeptide sequence.

Antigens can be selected that are predicted to be presented on the cell surface of a cell, such a as a tumor cell, an infected cell, or an immune cell, including professional antigen presenting cells such as dendritic cells. Antigens can be selected that are predicted to be immunogenic.

One or more polypeptides encoded by an antigen nucleotide sequence can comprise at least one of: a binding affinity with MHC with an IC50 value of less than 1000 nM, for MHC Class I peptides a length of 8-15, 8, 9, 10, 11, 12, 13, 14, or 15 amino acids, presence of sequence motifs within or near the peptide promoting proteasome cleavage, and presence or sequence motifs promoting TAP transport. For MHC Class II peptides a length 6-30, 6, 7, 8, 9, 10, 11, 12, 13, 14, 15, 16, 17, 18, 19, 20, 21, 22, 23, 24, 25, 26, 27, 28, 29, or 30 amino acids, presence of sequence motifs within or near the peptide promoting cleavage by extracellular or lysosomal proteases (e.g., cathepsins) or HLA-DM catalyzed HLA binding.

One or more antigens can be presented on the surface of a tumor.

One or more antigens can be immunogenic in a subject having a tumor, e.g., capable of stimulating a T cell response and/or a B cell response in the subject.

One or more antigens can be capable of stimulating a B cell response, such as the production of antibodies that recognize the one or more antigens (e.g., antibodies that recognize a tumor). Antibodies can recognize linear polypeptide sequences or recognize secondary and tertiary structures. Accordingly, B cell antigens can include linear polypeptide sequences or polypeptides having secondary and tertiary structures, including, but not limited to, full-length proteins, protein subunits, protein domains, or any polypeptide sequence known or predicted to have secondary and tertiary structures. Antigens capable of stimulating a B cell response to a tumor can be an antigen found on the surface of tumor cell or an infectious disease organism, respectively. Antigens capable of stimulating a B cell response to a tumor can be an intracellular neoantigen expressed in a tumor.

One or more antigens can include a combination of antigens capable of stimulating a T cell response (e.g., peptides including predicted T cell epitope sequences) and distinct antigens capable of stimulating a B cell response (e.g., full-length proteins, protein subunits, protein domains).

One or more antigens that stimulate an autoimmune response in a subject can be excluded from consideration in the context of vaccine generation for a subject.

The size of at least one antigenic peptide molecule (e.g., an epitope sequence) can comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120 or greater amino molecule residues, and any range derivable therein. In specific embodiments the antigenic peptide molecules are equal to or less than 50 amino acids.

Antigenic peptides and polypeptides can be: for MHC Class 115 residues or less in length and usually consist of between about 8 and about 11 residues, particularly 9 or 10 residues; for MHC Class II, 6-30 residues, inclusive.

If desirable, a longer peptide can be designed in several ways. In one case, when presentation likelihoods of peptides on HLA alleles are predicted or known, a longer peptide could consist of either: (1) individual presented peptides with an extensions of 2-5 amino acids toward the N- and C-terminus of each corresponding gene product; (2) a concatenation of some or all of the presented peptides with extended sequences for each. In another case, when sequencing reveals a long (>10 residues) neoepitope sequence present in the tumor (e.g. due to a frameshift, read-through or intron inclusion that leads to a novel peptide sequence), a longer peptide would consist of: (3) the entire stretch of novel tumor-specific or infectious disease-specific amino acids—thus bypassing the need for computational or in vitro test-based selection of the strongest HLA-presented shorter peptide. In both cases, use of a longer peptide allows endogenous processing by patient cells and may lead to more effective antigen presentation and stimulation of T cell responses. Longer peptides can also include a full-length protein, a protein subunit, a protein domain, and combinations thereof of a peptide, such as those expressed in a tumor or an infectious disease organism, respectively. Longer peptides (e.g., full-length protein, protein subunit, or protein domain) and combinations thereof can be included to stimulate a B cell response.

Antigenic peptides and polypeptides can be presented on an HLA protein. In some aspects antigenic peptides and polypeptides are presented on an HLA protein with greater affinity than a wild-type peptide. In some aspects, an antigenic peptide or polypeptide can have an IC50 of at least less than 5000 nM, at least less than 1000 nM, at least less than 500 nM, at least less than 250 nM, at least less than 200 nM, at least less than 150 nM, at least less than 100 nM, at least less than 50 nM or less.

In some aspects, antigenic peptides and polypeptides do not stimulate an autoimmune response and/or invoke immunological tolerance when administered to a subject.

Also provided are compositions comprising at least two or more antigenic peptides. In some embodiments the composition contains at least two distinct peptides. At least two distinct peptides can be derived from the same polypeptide. By distinct polypeptides is meant that the peptide vary by length, amino acid sequence, or both. A peptide can include a tumor-specific mutation. Tumor-specific peptides can be derived from any polypeptide known to or have been found to contain a tumor specific mutation or peptides derived from any polypeptide known to or have been found to have altered expression in a tumor cell or cancerous tissue in comparison to a normal cell or tissue, for example any polypeptide known to or have been found to be aberrantly expressed in a tumor cell or cancerous tissue in comparison to a normal cell or tissue. The peptides can be derived from any polypeptide known to or suspected to be associated with an infectious disease organism, or peptides derived from any polypeptide known to or have been found to have altered expression in an infected cell in comparison to a normal cell or tissue (e.g., an infectious disease polynucleotide or polypeptide, including infectious disease polynucleotides or polypeptides with expression restricted to a host cell). Suitable polypeptides from which the antigenic peptides can be derived can be found for example in the COSMIC database or the AACR Genomics Evidence Neoplasia Information Exchange (GENIE) database. COSMIC curates comprehensive information on somatic mutations in human cancer. AACR GENIE aggregates and links clinical-grade cancer genomic data with clinical outcomes from tens of thousands of cancer patients. In some aspects the tumor specific mutation is a driver mutation for a particular cancer type. Shared antigens with altered expression include, but are not limited to, CTA-associated mutations (e.g., a MAGEA1 MHC class I epitope, a MAGEA3 MHC class I epitope, a MAGEA4 MHC class I epitope, a MAGEA6 MHC class I epitope, a MAGEA11 MHC class I epitope, a MAGEB2 MHC class I epitope, and/or a CT83 MHC class I epitope).

Antigenic peptides and polypeptides having a desired activity or property can be modified to provide certain desired attributes, e.g., improved pharmacological characteristics, while increasing or at least retaining substantially all of the biological activity of the unmodified peptide to bind the desired MHC molecule and activate the appropriate T cell. For instance, antigenic peptide and polypeptides can be subject to various changes, such as substitutions, either conservative or non-conservative, where such changes might provide for certain advantages in their use, such as improved MHC binding, stability or presentation. By conservative substitutions is meant replacing an amino acid residue with another which is biologically and/or chemically similar, e.g., one hydrophobic residue for another, or one polar residue for another. The substitutions include combinations such as Gly, Ala; Val, Ile, Leu, Met; Asp, Glu; Asn, Gln; Ser, Thr; Lys, Arg; and Phe, Tyr. The effect of single amino acid substitutions may also be probed using D-amino acids. Such modifications can be made using well known peptide synthesis procedures, as described in e.g., Merrifield, Science 232:341-347 (1986), Barany & Merrifield, The Peptides, Gross & Meienhofer, eds. (N.Y., Academic Press), pp. 1-284 (1979); and Stewart & Young, Solid Phase Peptide Synthesis, (Rockford, Ill., Pierce), 2d Ed. (1984).

Modifications of peptides and polypeptides with various amino acid mimetics or unnatural amino acids can be particularly useful in increasing the stability of the peptide and polypeptide in vivo. Stability can be assayed in a number of ways. For instance, peptidases and various biological media, such as human plasma and serum, have been used to test stability. See, e.g., Verhoef et al., Eur. J. Drug Metab Pharmacokin. 11:291-302 (1986). Half-life of the peptides can be conveniently determined using a 25% human serum (v/v) assay. The protocol is generally as follows. Pooled human serum (Type AB, non-heat inactivated) is delipidated by centrifugation before use. The serum is then diluted to 25% with RPMI tissue culture media and used to test peptide stability. At predetermined time intervals a small amount of reaction solution is removed and added to either 6% aqueous trichloracetic acid or ethanol. The cloudy reaction sample is cooled (4 degrees C.) for 15 minutes and then spun to pellet the precipitated serum proteins. The presence of the peptides is then determined by reversed-phase HPLC using stability-specific chromatography conditions.

The peptides and polypeptides can be modified to provide desired attributes other than improved serum half-life. For instance, the ability of the peptides to stimulate CTL activity can be enhanced by linkage to a sequence which contains at least one epitope that is capable of stimulating a T helper cell response. Immunogenic peptides/T helper conjugates can be linked by a spacer molecule. The spacer is typically comprised of relatively small, neutral molecules, such as amino acids or amino acid mimetics, which are substantially uncharged under physiological conditions. The spacers are typically selected from, e.g., Ala, Gly, or other neutral spacers of nonpolar amino acids or neutral polar amino acids. It will be understood that the optionally present spacer need not be comprised of the same residues and thus can be a hetero- or homo-oligomer. When present, the spacer will usually be at least one or two residues, more usually three to six residues. Alternatively, the peptide can be linked to the T helper peptide without a spacer.

An antigenic peptide can be linked to the T helper peptide either directly or via a spacer either at the amino or carboxy terminus of the peptide. The amino terminus of either the antigenic peptide or the T helper peptide can be acylated. Exemplary T helper peptides include tetanus toxoid 830-843, influenza 307-319, malaria circumsporozoite 382-398 and 378-389.

Proteins or peptides can be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and can be found at computerized databases known to those of ordinary skill in the art. One such database is the National Center for Biotechnology Information's Genbank and GenPept databases located at the National Institutes of Health website. The coding regions for known genes can be amplified and/or expressed using the techniques disclosed herein or as would be known to those of ordinary skill in the art. Alternatively, various commercial preparations of proteins, polypeptides and peptides are known to those of skill in the art.

In a further aspect an antigen includes a nucleic acid (e.g. polynucleotide) that encodes an antigenic peptide or portion thereof. The polynucleotide can be, e.g., DNA, cDNA, PNA, CNA, RNA (e.g., mRNA), either single- and/or double-stranded, or native or stabilized forms of polynucleotides, such as, e.g., polynucleotides with a phosphorothioate backbone, or combinations thereof and it may or may not contain introns. A polynucleotide sequence encoding an antigen can be sequence-optimized to improve expression, such as through improving transcription, translation, post-transcriptional processing, and/or RNA stability. For example, polynucleotide sequence encoding an antigen can be codon-optimized. “Codon-optimization” herein refers to replacing infrequently used codons, with respect to codon bias of a given organism, with frequently used synonymous codons. Polynucleotide sequences can be optimized to improve post-transcriptional processing, for example optimized to reduce unintended splicing, such as through removal of splicing motifs (e.g., canonical and/or cryptic/non-canonical splice donor, branch, and/or acceptor sequences) and/or introduction of exogenous splicing motifs (e.g., splice donor, branch, and/or acceptor sequences) to bias favored splicing events. Exogenous intron sequences include, but are not limited to, those derived from SV40 (e.g., an SV40 mini-intron) and derived from immunoglobulins (e.g., human β-globin gene). Exogenous intron sequences can be incorporated between a promoter/enhancer sequence and the antigen(s) sequence. Exogenous intron sequences for use in expression vectors are described in more detail in Callendret et al. (Virology. 2007 Jul. 5; 363(2): 288-302), herein incorporated by reference for all purposes. Polynucleotide sequences can be optimized to improve transcript stability, for example through removal of RNA instability motifs (e.g., AU-rich elements and 3′ UTR motifs) and/or repetitive nucleotide sequences. Polynucleotide sequences can be optimized to improve accurate transcription, for example through removal of cryptic transcriptional initiators and/or terminators. Polynucleotide sequences can be optimized to improve translation and translational accuracy, for example through removal of cryptic AUG start codons, premature polyA sequences, and/or secondary structure motifs. Polynucleotide sequences can be optimized to improve nuclear export of transcripts, such as through addition of a Constitutive Transport Element (CTE), RNA Transport Element (RTE), or Woodchuck Posttranscriptional Regulatory Element (WPRE). Nuclear export signals for use in expression vectors are described in more detail in Callendret et al. (Virology. 2007 Jul. 5; 363(2): 288-302), herein incorporated by reference for all purposes. Polynucleotide sequences can be optimized with respect to GC content, for example to reflect the average GC content of a given organism. Sequence optimization can balance one or more sequence properties, such as transcription, translation, post-transcriptional processing, and/or RNA stability. Sequence optimization can generate an optimal sequence balancing each of transcription, translation, post-transcriptional processing, and RNA stability. Sequence optimization algorithms are known to those of skill in the art, such as GeneArt (Thermo Fisher), Codon Optimization Tool (IDT), Cool Tool (University of Singapore), SGI-DNA (La Jolla California). One or more regions of an antigen-encoding protein can be sequence-optimized separately.

A still further aspect provides an expression vector capable of expressing a polypeptide or portion thereof. Expression vectors for different cell types are well known in the art and can be selected without undue experimentation. Generally, DNA is inserted into an expression vector, such as a plasmid, in proper orientation and correct reading frame for expression. If necessary, DNA can be linked to the appropriate transcriptional and translational regulatory control nucleotide sequences recognized by the desired host, although such controls are generally available in the expression vector. The vector is then introduced into the host through standard techniques. Guidance can be found e.g. in Sambrook et al. (1989) Molecular Cloning, A Laboratory Manual, Cold Spring Harbor Laboratory, Cold Spring Harbor, N.Y.

### V. Vaccine Compositions

Also disclosed herein is an immunogenic composition, e.g., a vaccine composition, capable of raising a specific immune response, e.g., a tumor-specific immune response or an infectious disease organism-specific immune response. Vaccine compositions typically comprise one or a plurality of antigens, e.g., selected using a method described herein. Vaccine compositions can also be referred to as vaccines.

Vaccine compositions can include an antigen-encoding vaccine system. An antigen-encoding vaccine system can include any means to deliver one or more (neo)epitope-encoding nucleic acids. For example, a vaccine system can include (1) a single vector encoding each (neo)epitope-encoding nucleic acid to be delivered (e.g., a single vector encoding both a CTA-encoding nucleic acid sequence and a KRAS-encoding nucleic acid sequence); (2) a single vector including a single cassette encoding each (neo)epitope-encoding nucleic acid to be delivered (e.g., a single cassette encoding both a CTA-encoding nucleic acid sequence and a KRAS-encoding nucleic acid sequence); or (3) separate vectors that each encode separate and distinct (neo)epitope-encoding nucleic acids (e.g., a first vector encoding a CTA-encoding nucleic acid sequence and second vector encoding a KRAS-encoding nucleic acid sequence). In general, such vaccine systems refer to vaccine compositions designed to be co-delivered to a subject concurrently (e.g., co-delivered as a vaccine priming dose or co-delivered as a vaccine boosting dose), such as either administration of a single composition (e.g., injection of single vector or a mixture of separate vectors [a “blended” vaccine], which also includes multiple injections of a single vector or a mixture of separate vectors, such as bilateral administration of a single vector or a mixture of separate vectors), or co-administration of separate compositions.

A vaccine can contain between 1 and 30 peptides, 2, 3, 4, 5, 6, 7, 8, 9, 10, 11, 12, 13, 14, 15, 16, 17, 18, 19, 20, 21, 22, 23, 24, 25, 26, 27, 28, 29, or 30 different peptides, 6, 7, 8, 9, 10 11, 12, 13, or 14 different peptides, or 12, 13 or 14 different peptides. Peptides can include post-translational modifications. A vaccine can contain between 1 and 100 or more nucleotide sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different nucleotide sequences, 6, 7, 8, 9, 10 11, 12, 13, or 14 different nucleotide sequences, or 12, 13 or 14 different nucleotide sequences. A vaccine can contain between 1 and 30 antigen sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different antigen sequences, 6, 7, 8, 9, 10 11, 12, 13, or 14 different antigen sequences, or 12, 13 or 14 different antigen sequences.

A vaccine can contain between 1 and 30 antigen-encoding nucleic acid sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different antigen-encoding nucleic acid sequences, 6, 7, 8, 9, 10 11, 12, 13, or 14 different antigen-encoding nucleic acid sequences, or 12, 13 or 14 different antigen-encoding nucleic acid sequences. Antigen-encoding nucleic acid sequences can refer to the antigen encoding portion of an “antigen cassette.” Features of an antigen cassette are described in greater detail herein. An antigen-encoding nucleic acid sequence can contain one or more epitope-encoding nucleic acid sequences (e.g., an antigen-encoding nucleic acid sequence encoding concatenated T cell epitopes).

A vaccine can contain between 1 and 30 distinct epitope-encoding nucleic acid sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more distinct epitope-encoding nucleic acid sequences, 6, 7, 8, 9, 10 11, 12, 13, or 14 distinct epitope-encoding nucleic acid sequences, or 12, 13 or 14 distinct epitope-encoding nucleic acid sequences. Epitope-encoding nucleic acid sequences can refer to sequences for individual epitope sequences, such as each of the T cell epitopes in an antigen-encoding nucleic acid sequence encoding concatenated T cell epitopes.

A vaccine can contain at least two iterations of an epitope-encoding nucleic acid sequence. A used herein, an “iteration” (or interchangeably a “repeat”) refers to two or more identical nucleic acid epitope-encoding nucleic acid sequences (inclusive of the optional 5′ linker sequence and/or the optional 3′ linker sequences described herein) within an antigen-encoding nucleic acid sequence. In one example, the antigen-encoding nucleic acid sequence portion of a cassette encodes at least two iterations of an epitope-encoding nucleic acid sequence. In further non-limiting examples, the antigen-encoding nucleic acid sequence portion of a cassette encodes more than one distinct epitope, and at least one of the distinct epitopes is encoded by at least two iterations of the nucleic acid sequence encoding the distinct epitope (i.e., at least two distinct epitope-encoding nucleic acid sequences). In illustrative non-limiting examples, an antigen-encoding nucleic acid sequence encodes epitopes A, B, and C encoded by epitope-encoding nucleic acid sequences epitope-encoding sequence A (E_{A}), epitope-encoding sequence B (E_{B}), and epitope-encoding sequence C (E_{C}), and exemplary antigen-encoding nucleic acid sequences having iterations of at least one of the distinct epitopes are illustrated by, but is not limited to, the formulas below:
Repeat of one distinct epitope (repeat of epitope A):

E_{A}-E_{B}-E_{C}-E_{A}; or

E_{A}-E_{A}-E_{B}-E_{C}
Repeat of multiple distinct epitopes (iterations of epitopes A, B, and C):

E_{A}-E_{B}-E_{C}-E_{A}-E_{B}-E_{C}; or

E_{A}-E_{A}-E_{B}-E_{B}-E_{C}-E_{C}
Multiple iterations of multiple distinct epitopes (iterations of epitopes A, B, and C):

E_{A}-E_{B}-E_{C}-E_{A}-E_{B}-E_{C}-E_{A}-E_{B}-E_{C}; or

E_{A}-E_{A}-E_{A}-E_{B}-E_{B}-E_{B}-E_{C}-E_{C}-E_{C}

The above examples are not limiting and the antigen-encoding nucleic acid sequences having iterations of at least one of the distinct epitopes can encode each of the distinct epitopes in any order or frequency. For example, the order and frequency can be a random arrangement of the distinct epitopes, e.g., in an example with epitopes A, B, and C, by the formula E_{A}-E_{B}-E_{C}-E_{C}-E_{A}-E_{B}-E_{A}-E_{C}-E_{A}-E_{C}-E_{C}-E_{B}.

An illustrative antigen-encoding cassette design having two or more distinct and non-identical MHC epitopes is described by the following where the antigen-encoding cassette includes:
(i) a nucleic acid sequence A (E_{A}); and
(ii) a nucleic acid sequence B (E_{B}),
wherein E_{A} and E_{B} each encode one MHC epitope,
wherein the MHC epitope encoded by E_{A} and the MHC epitope encoded by E_{B} are distinct and non-identical.

The cassette can further include a nucleic acid sequence C (E_{C}), wherein E_{C} encodes one MHC epitope, wherein the MHC epitope encoded by E_{C} is and distinct and non-identical with respect to the MHC epitope encoded by E_{A} and the MHC epitope encoded by E_{B}.

An illustrative antigen-encoding cassette design having two or more iterations of two or more distinct and non-identical MHC epitopes is described by the following where the antigen-encoding cassette includes:
(i) a nucleic acid sequence A (E_{A}); and
(ii) a nucleic acid sequence B (E_{B}),
wherein E_{A} and E_{B} each encode one MHC epitope,
wherein the MHC epitope encoded by E_{A} and the MHC epitope encoded by E_{B} are distinct and non-identical,
wherein the cassette includes at least two iterations of E_{A} and at least two iterations of E, and
wherein each iteration of E_{A} and E_{B}, respectively, are identical nucleic acid sequences.

The cassette above can encode at least 3, at least 4, at least 5, at least 6, at least 7, or at least 8 iterations of one or both of E_{A} and E_{B}. The cassette above can encode at least 8 iterations of one or both of E_{A} and E_{B}. The cassette above can encode 2 iterations of one or both of E_{A} and E_{B}. The cassette above can encode 3 iterations of one or both of E_{A} and E_{B}. The cassette above can encode 4 iterations of one or both of E_{A} and E_{B}. The cassette above can encode 5 iterations of one or both of E_{A} and E_{B}. The cassette above can encode 6 iterations of one or both of E_{A} and E_{B}. The cassette above can encode 7 iterations of one or both of E_{A} and E_{B}. The cassette above can encode 8 iterations of one or both of E_{A} and E_{B}.

The cassette above can encode at least 3, at least 4, at least 5, at least 6, at least 7, or at least 8 iterations of each of E_{A} and E_{B}. The cassette above can encode at least 8 iterations of each of E_{A} and E_{B}. The cassette above can encode 2 iterations of each of E_{A} and E_{B}. The cassette above can encode 3 iterations of each of E_{A} and E_{B}. The cassette above can encode 4 iterations of each of E_{A} and E_{B}. The cassette above can encode 5 iterations of each of E_{A} and E_{B}. The cassette above can encode 6 iterations of each of E_{A} and E_{B}. The cassette above can encode 7 iterations of each of E_{A} and E_{B}. The cassette above can encode 8 iterations of each of E_{A} and E_{B}.

E_{A} and/or E_{B} can be concatenated to nucleic acids encoding other MHC epitopes (e.g., other E_{A} and/or E_{B}) by a linker-encoding nucleic acid.

The cassette above can encode each of E_{A} and E_{B} in a unit E_{A}-E_{B} that is repeated, e.g., the unit E_{A}-E_{B} repeated 4 times is illustrated by the following: E_{A}-E_{B}-E_{A}-E_{B}-E_{A}-E_{B}-E_{A}-E_{B}.

The cassette above can further include a nucleic acid sequence C (E_{C}), where E_{C} encodes one MHC epitope, where the MHC epitope encoded by E_{C} is and distinct and non-identical with respect to the MHC epitope encoded by E_{A} and the MHC epitope encoded by E_{B}, where the cassette comprises at least two iterations of E_{C} and, and where each iteration of E_{C} is an identical nucleic acid sequence. The cassette above can encode each of the unit E_{A}-E_{B}-E_{C} that is repeated, e.g., the unit E_{A}-E_{B}-E_{C} repeated 4 times is illustrated by the following: E_{A}-E_{B}-E_{C}-E_{A}-E_{B}-E_{C}-E_{A}-E_{B}-E_{C}-E_{A}-E_{B}-E_{C}.

The cassette above can encode at least 3, at least 4, at least 5, at least 6, at least 7, or at least 8 iterations of each of E_{A}, E_{B}, and E_{C}. The cassette above can encode at least 8 iterations of each of E_{A}, E_{B}, and E_{C}. The cassette above can encode 2 iterations of each of E_{A}, E_{B}, and E_{C}. The cassette above can encode 3 iterations of each of E_{A}, E_{B}, and E_{C}. The cassette above can encode 4 iterations of each of E_{A}, E_{B}, and E_{C}. The cassette above can encode 5 iterations of each of E_{A}, E_{B}, and E_{C}. The cassette above can encode 6 iterations of each of E_{A}, E_{B}, and E_{C}. The cassette above can encode 7 iterations of each of E_{A}, E_{B}, and E_{C}. The cassette above can encode 8 iterations of each of E_{A}, E_{B}, and E_{C}.

While iterations of E_{A}, E_{B}, and/or E_{C} may be repeated in a unit, such as E_{A}-E_{B}-E_{C}, they may be repeated in alternative orders, e.g. E_{A}-E_{B}-E_{C}-E_{C}-E_{B}-E_{A}-E_{A}-E_{B}-E_{C}-E_{B}-E_{A}-E_{C} or E_{A}-E_{A}-E_{A}-E_{A}-E_{B}-E_{B}-E_{B}-E_{B}-E_{C}-E_{C}-E_{C}-E_{C}, including orders designed to minimize junctional epitope formation.

E_{A}, E_{B}, and/or E_{C} can be concatenated to nucleic acids encoding other MHC epitopes (e.g., other E_{A}, E_{B}, and/or E_{C}) by a linker-encoding nucleic acid.

Also provided for herein is an antigen-encoding cassette, the antigen-encoding cassette having at least one antigen-encoding nucleic acid sequence described, from 5′ to 3′, by the formula:

(Eₓ-(E^{N}ₙ)_{Y})_{z}
where E represents a nucleotide sequence including a distinct epitope-encoding nucleic acid sequence,
n represents the number of separate distinct epitope-encoding nucleic acid sequences and is any integer including 0,
E^{N} represents a nucleotide sequence comprising the separate distinct epitope-encoding nucleic acid sequence for each corresponding n,
for each iteration of z: x=0 or 1, y=0 or 1 for each n, and at least one of x or y=1, and
z=1 or greater, optionally wherein the antigen-encoding nucleic acid sequence comprises at least two iterations of E, a given E^{N}, or a combination thereof, and
   at least one of the distinct epitope-encoding nucleic acid sequences encodes a distinct shared epitope, such as a Cancer Testis Antigen (CTA)-associated MHC class I epitope and/or a KRAS-associated MHC class I neoepitope, optionally wherein at least one of the distinct epitope-encoding nucleic acid sequences, e.g., those encoding the CTA-associated MHC class I epitope and/or the KRAS-associated MHC class I neoepitope, comprises at least two iterations.

Each E or E^{N} can independently comprise any epitope-encoding nucleic acid sequence described herein. For example, Each E or E^{N} can independently comprises a nucleotide sequence described, from 5′ to 3′, by the formula (L5_{b}-N_{c}-L3_{d}), where N comprises the distinct epitope-encoding nucleic acid sequence associated with each E or E^{N}, where c=1, L5 comprises a 5′ linker sequence, where b=0 or 1, and L3 comprises a 3′ linker sequence, where d=0 or 1. Epitopes and linkers that can be used are further described herein.

Iterations of an epitope-encoding nucleic acid sequences (inclusive of optional 5′ linker sequence and/or the optional 3′ linker sequences) can be linearly linked directly to one another (e.g., E_{A}-E_{A}- . . . as illustrated above). Iterations of an epitope-encoding nucleic acid sequences can be separated by one or more additional nucleotides sequences. In general, iterations of an epitope-encoding nucleic acid sequences can be separated by any size nucleotide sequence applicable for the compositions described herein. In one example, iterations of an epitope-encoding nucleic acid sequences can be separated by a separate distinct epitope-encoding nucleic acid sequence (e.g., E_{A}-E_{B}-E_{C}-E_{A} . . . , as illustrated above). In examples where iterations are separated by a single separate distinct epitope-encoding nucleic acid sequence, and each epitope-encoding nucleic acid sequences (inclusive of optional 5′ linker sequence and/or the optional 3′ linker sequences) encodes a peptide 25 amino acids in length, the iterations can be separated by 75 nucleotides, such as in antigen-encoding nucleic acid represented by E_{A}-E_{B}-E_{A} . . . , E_{A} is separated by 75 nucleotides. In an illustrative example, an antigen-encoding nucleic acid having the sequence VTNTEMFVTAPDNLGYMYEVQWPGQTQPQIANCSVYDFFVWLHYYSVRDTVTNTEMFVT APDNLGYMYEVQWPGQTQPQIANCSVYDFFVWLHYYSVRDT encoding iterations of 25mer antigens Trp1 (VTNTEMFVTAPDNLGYMYEVQWPGQ) and Trp2 (TQPQIANCSVYDFFVWLHYYSVRDT), the iterations of Trp1 are separated by the 25mer Trp2 and thus the repeats of the Trp1 epitope-encoding nucleic acid sequences are separated the 75 nucleotide Trp2 epitope-encoding nucleic acid sequence. In examples where iterations are separated by 2, 3, 4, 5, 6, 7, 8, or 9 separate distinct epitope-encoding nucleic acid sequence, and each epitope-encoding nucleic acid sequences (inclusive of optional 5′ linker sequence and/or the optional 3′ linker sequences) encodes a peptide 25 amino acids in length, the iterations can be separated by 150, 225, 300, 375, 450, 525, 600, or 675 nucleotides, respectively.

In one embodiment, different peptides and/or polypeptides or nucleotide sequences encoding them are selected so that the peptides and/or polypeptides capable of associating with different MHC molecules, such as different MHC class I molecules and/or different MHC class II molecules. In some aspects, one vaccine composition comprises coding sequence for peptides and/or polypeptides capable of associating with the most frequently occurring MHC class I molecules and/or different MHC class II molecules. Hence, vaccine compositions can comprise different fragments capable of associating with at least 2 preferred, at least 3 preferred, or at least 4 preferred MHC class I molecules and/or different MHC class II molecules.

The composition can be capable of stimulating a specific cytotoxic T-cell response and/or a specific helper T-cell response. The vaccine composition can be capable of stimulating a specific cytotoxic T-cell response and a specific helper T-cell response.

The vaccine composition can be capable of stimulating a specific B-cell response (e.g., an antibody response).

The vaccine composition can be capable of stimulating a specific cytotoxic T-cell response, a specific helper T-cell response, and/or a specific B-cell response. The vaccine composition can be capable of stimulating a specific cytotoxic T-cell response and a specific B-cell response. The vaccine composition can be capable of stimulating a specific helper T-cell response and a specific B-cell response. The vaccine composition can be capable of stimulating a specific cytotoxic T-cell response, a specific helper T-cell response, and a specific B-cell response.

A vaccine composition can further comprise an adjuvant and/or a carrier. Examples of useful adjuvants and carriers are given herein below. A composition can be associated with a carrier such as e.g. a protein or an antigen-presenting cell such as, e.g., a dendritic cell (DC) capable of presenting the peptide to a T-cell.

Adjuvants are any substance whose admixture into a vaccine composition increases or otherwise modifies the immune response to an antigen. Carriers can be scaffold structures, for example a polypeptide or a polysaccharide, to which an antigen, is capable of being associated. Optionally, adjuvants are conjugated covalently or non-covalently.

The ability of an adjuvant to increase an immune response to an antigen is typically manifested by a significant or substantial increase in an immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th response into a primarily cellular, or Th response.

Suitable adjuvants include, but are not limited to 1018 ISS, alum, aluminium salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel vector system, PLG microparticles, resiquimod, SRL172, Virosomes and other Virus-like particles, YF-17D, VEGF trap, R848, beta-glucan, Pam3Cys, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Adjuvants such as incomplete Freund's or GM-CSF are useful. Several immunological adjuvants (e.g., MF59) specific for dendritic cells and their preparation have been described previously (Dupuis M, et al., Cell Immunol. 1998; 186(1):18-27; Allison A C; Dev Biol Stand. 1998; 92:3-11). Also cytokines can be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12) (Gabrilovich D I, et al., J Immunother Emphasis Tumor Immunol. 1996 (6):414-418).

CpG immunostimulatory oligonucleotides have also been reported to enhance the effects of adjuvants in a vaccine setting. Other TLR binding molecules such as RNA binding TLR 7, TLR 8 and/or TLR 9 may also be used.

Other examples of useful adjuvants include, but are not limited to, chemically modified CpGs (e.g. CpR, Idera), Poly(I:C) (e.g. polyi:CI2U), non-CpG bacterial DNA or RNA as well as immunoactive small molecules and antibodies such as cyclophosphamide, sunitinib, bevacizumab, celebrex, NCX-4016, sildenafil, tadalafil, vardenafil, sorafinib, XL-999, CP-547632, pazopanib, ZD2171, AZD2171, ipilimumab, tremelimumab, and SC58175, which may act therapeutically and/or as an adjuvant. The amounts and concentrations of adjuvants and additives can readily be determined by the skilled artisan without undue experimentation. Additional adjuvants include colony-stimulating factors, such as Granulocyte Macrophage Colony Stimulating Factor (GM-CSF, sargramostim).

A vaccine composition can comprise more than one different adjuvant. Furthermore, a therapeutic composition can comprise any adjuvant substance including any of the above or combinations thereof. It is also contemplated that a vaccine and an adjuvant can be administered together or separately in any appropriate sequence.

A carrier (or excipient) can be present independently of an adjuvant. The function of a carrier can for example be to increase the molecular weight of in particular mutant to increase activity or immunogenicity, to confer stability, to increase the biological activity, or to increase serum half-life. Furthermore, a carrier can aid presenting peptides to T-cells. A carrier can be any suitable carrier known to the person skilled in the art, for example a protein or an antigen presenting cell. A carrier protein could be but is not limited to keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier is generally a physiologically acceptable carrier acceptable to humans and safe. However, tetanus toxoid and/or diphtheria toxoid are suitable carriers. Alternatively, the carrier can be dextrans for example sepharose.

Cytotoxic T-cells (CTLs) recognize an antigen in the form of a peptide bound to an MHC molecule rather than the intact foreign antigen itself. The MHC molecule itself is located at the cell surface of an antigen presenting cell. Thus, an activation of CTLs is possible if a trimeric complex of peptide antigen, MHC molecule, and APC is present. Correspondingly, it may enhance the immune response if not only the peptide is used for activation of CTLs, but if additionally APCs with the respective MHC molecule are added. Therefore, in some embodiments a vaccine composition additionally contains at least one antigen presenting cell.

Antigens can also be included in viral vector-based vaccine platforms, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In Vivo Gene Delivery, J. Virol. (1998) 72 (12): 9873-9880). Dependent on the packaging capacity of the above mentioned viral vector-based vaccine platforms, this approach can deliver one or more nucleotide sequences that encode one or more antigen peptides. The sequences may be flanked by non-mutated sequences, may be separated by linkers or may be preceded with one or more sequences targeting a subcellular compartment (See, e.g., Gros et al., Prospective identification of neoantigen-specific lymphocytes in the peripheral blood of melanoma patients, Nat Med. (2016) 22 (4):433-8, Stronen et al., Targeting of cancer neoantigens with donor-derived T cell receptor repertoires, Science. (2016) 352 (6291):1337-41, Lu et al., Efficient identification of mutated cancer antigens recognized by T cells associated with durable tumor regressions, Clin Cancer Res. (2014) 20(13):3401-10). Upon introduction into a host, infected cells express the antigens, and thereby elicit a host immune (e.g., CTL) response against the peptide(s). Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vaccine vectors useful for therapeutic administration or immunization of antigens, e.g., Salmonella typhi vectors, and the like will be apparent to those skilled in the art from the description herein.

### V.A. Antigen Cassette

The methods employed for the selection of one or more antigens, the cloning and construction of an “antigen cassette” and its insertion into a viral vector are within the skill in the art given the teachings provided herein. By “antigen cassette” or “cassette” is meant the combination of a selected antigen or plurality of antigens (e.g., antigen-encoding nucleic acid sequences) and the other regulatory elements necessary to transcribe the antigen(s) and express the transcribed product. The selected antigen or plurality of antigens can refer to distinct epitope sequences, e.g., an antigen-encoding nucleic acid sequence in the cassette can encode an epitope-encoding nucleic acid sequence (or plurality of epitope-encoding nucleic acid sequences) such that the epitopes are transcribed and expressed. An antigen or plurality of antigens can be operatively linked to regulatory components in a manner which permits transcription. Such components include conventional regulatory elements that can drive expression of the antigen(s) in a cell transfected with the viral vector. Thus the antigen cassette can also contain a selected promoter which is linked to the antigen(s) and located, with other, optional regulatory elements, within the selected viral sequences of the recombinant vector. A cassette can have one or more antigen-encoding nucleic acid sequences, such as a cassette containing multiple antigen-encoding nucleic acid sequences each independently operably linked to separate promoters and/or linked together using other multicistonic systems, such as 2A ribosome skipping sequence elements (e.g., E2A, P2A, F2A, or T2A sequences) or Internal Ribosome Entry Site (IRES) sequence elements. A linker can also have a cleavage site, such as a TEV or furin cleavage site. Linkers with cleavage sites can be used in combination with other elements, such as those in a multicistronic system. In a non-limiting illustrative example, a furin protease cleavage site can be used in conjunction with a 2A ribosome skipping sequence element such that the furin protease cleavage site is configured to facilitate removal of the 2A sequence following translation. In a cassette containing more than one antigen-encoding nucleic acid sequences, each antigen-encoding nucleic acid sequence can contain one or more epitope-encoding nucleic acid sequences (e.g., an antigen-encoding nucleic acid sequence encoding concatenated T cell epitopes).

Useful promoters can be constitutive promoters or regulated (inducible) promoters, which will enable control of the amount of antigen(s) to be expressed. For example, a desirable promoter is that of the cytomegalovirus immediate early promoter/enhancer [see, e.g., Boshart et al, Cell, 41:521-530 (1985)]. Another desirable promoter includes the Rous sarcoma virus LTR promoter/enhancer. Still another promoter/enhancer sequence is the chicken cytoplasmic beta-actin promoter [T. A. Kost et al, Nucl. Acids Res., 11(23):8287 (1983)]. Other suitable or desirable promoters can be selected by one of skill in the art.

Also disclosed herein is a viral vector comprising a cassette with at least one payload sequence operably linked to a regulatable promoter that is a TET promoter system, such as a TET-On system or TET-Off system. Without wishing to be bound by theory, a TET promoter system can be used to minimize transcription of payload nucleic acids encoded in a cassette, such as antigens encoded in a vaccine cassette, during viral production. TET promoter systems are described in detail in international patent application publication WO2020/243719, herein incorporated by reference for all purposes.

A TET promoter system can include a tetracycline (TET) repressor protein (TETr) controlled promoter. Accordingly, also disclosed herein is a viral vector comprising a cassette with at least one payload sequence operably linked to a tetracycline (TET) repressor protein (TETr) controlled promoter. A TETr controlled promoter can include the 19 bp TET operator (TETo) sequence TCCCTATCAGTGATAGAGA (SEQ ID NO:10,756). A TETr controlled promoter can include 2, 3, 4, 5, 6, 7, 8, 9, or 10 or more TETo nucleic acid sequences. In TETr controlled promoter have 2 or more TETo nucleic acid sequences, the TETo sequences can be linked together. In TETr controlled promoter have 2 or more TETo nucleic acid sequences, the TETo sequences can be directly linked together. In TETr controlled promoter have 2 or more TETo nucleic acid sequences, the TETo sequences can be linked together with a linker sequence, such as a linker sequence having 1, 2, 3, 4, 5, 6, 7, 8, 9, 10, 11, 12, 13, 14, 15, 16, 17, 18, 19, or 20 or more nucleotides. In general, a TETr controlled promoter can use any promoter sequence desired, such as a SV40, EF-1, RSV, PGK, HSA, MCK or EBV promoter sequence. A TETr controlled promoter can use a CMV promoter sequence. A TETr controlled promoter can use a minimal CMV promoter sequence. TETo sequences can be upstream (5′) of a promoter sequence region where RNA polymerase binds. In an illustrative example, 7 TETo sequences are upstream (5′) of a promoter sequence. A TETr controlled promoter operably linked to the at least one payload nucleic acid sequence with TETo sequence upstream of the promoter sequence region can have an ordered sequence described in the formula, from 5′ to 3′:

(T-L_{Y})_{X}-P-N

where N is a payload nucleic acid sequence, P is a RNA polymerase binding sequence of the promoter sequence operably linked to payload nucleic acid sequence, T is a TETo nucleic acid sequences comprising the nucleotide sequence shown in SEQ ID NO:10,756, L is a linker sequence, where Y=0 or 1 for each X, and wherein X=1, 2, 3, 4, 5, 6, 7, 8, 9, 10, 11, 12, 13, 14, 15, 16, 17, 18, 19, or 20. In an illustrative example, X=7 and Y=1 for each X describes where 7 TETo sequences are upstream (5′) of the promoter sequence and each TETo sequence is separated by a linker.

A TETo sequences can be downstream (3′) of a promoter sequence region where RNA polymerase binds. In another illustrative example, 2 TETo sequences are downstream (3′) of a promoter sequence. A TETr controlled promoter operably linked to the at least one payload nucleic acid sequence with TETo sequence downstream of the promoter sequence region can have an ordered sequence described in the formula, from 5′ to 3′:

P-(T-L_{Y})_{X}-N

where N is a payload nucleic acid sequence, P is a RNA polymerase binding sequence of the promoter sequence operably linked to payload nucleic acid sequence, T is a TETo nucleic acid sequences comprising the nucleotide sequence shown in SEQ ID NO:10,756, L is a linker sequence, where Y=0 or 1 for each X, and wherein X=1, 2, 3, 4, 5, 6, 7, 8, 9, 10, 11, 12, 13, 14, 15, 16, 17, 18, 19, or 20. In an illustrative example, X=2 and Y=1 for each X describes where 2 TETo sequences are downstream (3′) of the promoter sequence and each TETo sequence is separated by a linker.

Viral production of vectors with TETr controlled promoters can use any viral production cell line engineered to express a TETr sequence (tTS), such as a 293 cell line or its derivatives (e.g., a 293F cell line) engineered to express tTS. Viral production of vectors with TETr controlled promoters in tTS-expressing cell can improve viral production. Viral production of vectors with TETr controlled promoters in tTS-expressing cell can improve viral infectivity defined as viral particles (VP) per infectious unit (IU). Viral production of vectors with TETr controlled promoters in tTS-expressing cell can improve viral production and/or viral infectivity by at least 1.5, at least 2, at least 2.5, at least 3, at least 3.5, at least 4, at least 4.5, at least 5, at least 6, at least 7, at least 8, at least 9, or at least 10-fold relative to production in a non-tTS-expressing cell. Viral production of vectors with TETr controlled promoters in tTS-expressing cell can improve viral production and/or viral infectivity by at least 15, at least 20, at least 25, at least 30, at least 35, at least 40, at least 45, at least 50, at least 60, at least 70, at least 80, at least 90, or at least 100-fold relative to production in a non-tTS-expressing cell. Viral production of vectors with TETr controlled promoters in tTS-expressing cell can improve viral production and/or viral infectivity by at least 1.5, at least 2, at least 2.5, at least 3, at least 3.5, at least 4, at least 4.5, at least 5, at least 6, at least 7, at least 8, at least 9, or at least 10-fold relative to production of a vector not having a TETr controlled promoter. Viral production of vectors with TETr controlled promoters in tTS-expressing cell can improve viral production and/or viral infectivity by at least 15, at least 20, at least 25, at least 30, at least 35, at least 40, at least 45, at least 50, at least 60, at least 70, at least 80, at least 90, or at least 100-fold relative to production of a vector not having a TETr controlled promoter.

The antigen cassette can also include nucleic acid sequences heterologous to the viral vector sequences including sequences providing signals for efficient polyadenylation of the transcript (poly(A), poly-A or pA) and introns with functional splice donor and acceptor sites. A common poly-A sequence which is employed in the exemplary vectors of this invention is that derived from the papovavirus SV-40. The poly-A sequence generally can be inserted in the cassette following the antigen-based sequences and before the viral vector sequences. A common intron sequence can also be derived from SV-40, and is referred to as the SV-40 T intron sequence. An antigen cassette can also contain such an intron, located between the promoter/enhancer sequence and the antigen(s). Selection of these and other common vector elements are conventional [see, e.g., Sambrook et al, “Molecular Cloning. A Laboratory Manual.”, 2d edit., Cold Spring Harbor Laboratory, New York (1989) and references cited therein] and many such sequences are available from commercial and industrial sources as well as from Genbank.

An antigen cassette can have one or more antigens. For example, a given cassette can include 1-10, 1-20, 1-30, 10-20, 15-25, 15-20, 1, 2, 3, 4, 5, 6, 7, 8, 9, 10, 11, 12, 13, 14, 15, 16, 17, 18, 19, 20, or more antigens. Antigens can be linked directly to one another. Antigens can also be linked to one another with linkers. Antigens can be in any orientation relative to one another including N to C or C to N.

As above stated, the antigen cassette can be located in the site of any selected deletion in the viral vector, such as the site of the E1 gene region deletion or E3 gene region deletion, among others which may be selected.

The antigen cassette can be described using the following formula to describe the ordered sequence of each element, from 5′ to 3′:

(Pₐ-(L5_{b}-N_{c}-L3_{d})_{X})_{Z}-(P2ₕ-(G5ₑ-U_{f})_{Y})_{W}-G3_{g}

wherein P and P2 comprise promoter nucleotide sequences, N comprises a distinct epitope-encoding nucleic acid sequence, L5 comprises a 5′ linker sequence, L3 comprises a 3′ linker sequence, G5 comprises a nucleic acid sequences encoding an amino acid linker, G3 comprises one of the at least one nucleic acid sequences encoding an amino acid linker, U comprises an MHC class II antigen-encoding nucleic acid sequence, where for each X the corresponding Nc is an epitope encoding nucleic acid sequence, where for each Y the corresponding Uf is a MHC class II epitope-encoding nucleic acid sequence (e.g., universal MHC class II epitope-encoding nucleic acid sequence). A universal sequence can comprise at least one of Tetanus toxoid and PADRE. A universal sequence can comprise a Tetanus toxoid peptide. A universal sequence can comprise a PADRE peptide. A universal sequence can comprise a Tetanus toxoid and PADRE peptides. The composition and ordered sequence can be further defined by selecting the number of elements present, for example where a=0 or 1, where b=0 or 1, where c=1, where d=0 or 1, where e=0 or 1, where f=1, where g=0 or 1, where h=0 or 1, X=1 to 400, Y=0, 1, 2, 3, 4 or 5, Z=1 to 400, and W=0, 1, 2, 3, 4 or 5.

In one example, elements present include where a=0, b=1, d=1, e=1, g=1, h=0, X=10, Y=2, Z=1, and W=1, describing where no additional promoter is present (e.g. only the promoter nucleotide sequence provided by a vector backbone, such as an RNA alphavirus backbone is present), 10 epitopes are present, a 5′ linker is present for each N, a 3′ linker is present for each N, 2 MHC class II epitopes are present, a linker is present linking the two MHC class II epitopes, a linker is present linking the 5′ end of the two MHC class II epitopes to the 3′ linker of the final MHC class I epitope, and a linker is present linking the 3′ end of the two MHC class II epitopes to a vector backbone (e.g., an RNA alphavirus backbone). Examples of linking the 3′ end of the antigen cassette to a vector backbone (e.g., an RNA alphavirus backbone) include linking directly to the 3′ UTR elements provided by the vector backbone, such as a 3′ 19-nt CSE. Examples of linking the 5′ end of the antigen cassette to a vector backbone (e.g., an RNA alphavirus backbone) include linking directly to a promoter or 5′ UTR element of the vector backbone, such as a subgenomic promoter sequence (e.g., a 26S subgenomic promoter sequence), an alphavirus 5′ UTR, a 51-nt CSE, or a 24-nt CSE.

Other examples include: where a=1 describing where a promoter other than the promoter nucleotide sequence provided a vector backbone (e.g., an RNA alphavirus backbone) is present; where a=1 and Z is greater than 1 where multiple promoters other than the promoter nucleotide sequence provided by the vector backbone are present each driving expression of 1 or more distinct MHC class I epitope encoding nucleic acid sequences; where h=1 describing where a separate promoter is present to drive expression of the MHC class II epitope-encoding nucleic acid sequences; and where g=0 describing the MHC class II epitope-encoding nucleic acid sequence, if present, is directly linked to a vector backbone (e.g., an RNA alphavirus backbone).

Other examples include where each MHC class I epitope that is present can have a 5′ linker, a 3′ linker, neither, or both. In examples where more than one MHC class I epitope is present in the same antigen cassette, some MHC class I epitopes may have both a 5′ linker and a 3′ linker, while other MHC class I epitopes may have either a 5′ linker, a 3′ linker, or neither. In other examples where more than one MHC class I epitope is present in the same antigen cassette, some MHC class I epitopes may have either a 5′ linker or a 3′ linker, while other MHC class I epitopes may have either a 5′ linker, a 3′ linker, or neither.

In examples where more than one MHC class II epitope is present in the same antigen cassette, some MHC class II epitopes may have both a 5′ linker and a 3′ linker, while other MHC class II epitopes may have either a 5′ linker, a 3′ linker, or neither. In other examples where more than one MHC class II epitope is present in the same antigen cassette, some MHC class II epitopes may have either a 5′ linker or a 3′ linker, while other MHC class II epitopes may have either a 5′ linker, a 3′ linker, or neither.

Other examples include where each antigen that is present can have a 5′ linker, a 3′ linker, neither, or both. In examples where more than one antigen is present in the same antigen cassette, some antigens may have both a 5′ linker and a 3′ linker, while other antigens may have either a 5′ linker, a 3′ linker, or neither. In other examples where more than one antigen is present in the same antigen cassette, some antigens may have either a 5′ linker or a 3′ linker, while other antigens may have either a 5′ linker, a 3′ linker, or neither.

The promoter nucleotide sequences P and/or P2 can be the same as a promoter nucleotide sequence provided by a vector backbone, such as an RNA alphavirus backbone. For example, the promoter sequence provided by the vector backbone, Pn and P2, can each comprise a subgenomic promoter sequence (e.g., a 26S subgenomic promoter sequence) or a CMV promoter. The promoter nucleotide sequences P and/or P2 can be different from the promoter nucleotide sequence provided by a vector backbone (e.g., an RNA alphavirus backbone), as well as can be different from each other.

The 5′ linker L5 can be a native sequence or a non-natural sequence. Non-natural sequence include, but are not limited to, AAY, RR, and DPP. The 3′ linker L3 can also be a native sequence or a non-natural sequence. Additionally, L5 and L3 can both be native sequences, both be non-natural sequences, or one can be native and the other non-natural. For each X, the amino acid linkers L5 and/or L3 can each independently be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length. For each X, the amino acid linkers L5 and/or L3 can each independently be at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length. For each X, the amino acid linkers L5 and/or L3 can each independently be at least 5 amino acids in length. For each X, the amino acid linkers L5 and/or L3 can each independently be at least 6 amino acids in length. For each X, the amino acid linkers L5 and/or L3 can each independently be at least 7 amino acids in length. For each X, the amino acid linkers L5 and/or L3 can each independently be at least 8 amino acids in length. For each X, the amino acid linkers L5 and/or L3 can each independently be at least 9 amino acids in length. For each X, the amino acid linkers L5 and/or L3 can each independently be between 2-20, between 5-20, between 6-20, between 7-20, between 8-20, between 9-20, between 10-20 amino acids in length. For each X, the amino acid linkers L5 and/or L3 can each independently be between 2-15, between 5-20, between 6-20, between 7-20, between 8-20, between 9-20, between 10-20 amino acids in length.

The amino acid linker G5, for each Y, can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length. For each Y, the amino acid linkers can be also be at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length.

The amino acid linker G3 can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length. G3 can be also be at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length.

For each X, each N can encode a MHC class I epitope, a MHC class II epitope, an epitope/antigen capable of stimulating a B cell response, or a combination thereof. For each X, each N can encode a combination of a MHC class I epitope, a MHC class II epitope, and an epitope/antigen capable of stimulating a B cell response. For each X, each N can encode a combination of a MHC class I epitope and a MHC class II epitope. For each X, each N can encode a combination of a MHC class I epitope and an epitope/antigen capable of stimulating a B cell response. For each X, each N can encode a combination of a MHC class II epitope and an epitope/antigen capable of stimulating a B cell response. For each X, each N can encode a MHC class II epitope. For each X, each N can encode an epitope/antigen capable of stimulating a B cell response. For each X, each N can encode a MHC class I epitope 7-15 amino acids in length. For each X, each N can encode a MHC class I epitope 5, 6, 7, 8, 9, 10, 11, 12, 13, 14, 15, 16, 17, 18, 19, 20, 21, 22, 23, 24, 25, 26, 27, 28, 29, or 30 amino acids in length. For each X, each N can encode a MHC class I epitope at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length.

The cassette encoding the one or more antigens can be 700 nucleotides or less. The cassette encoding the one or more antigens can be 700 nucleotides or less and encode 2 distinct epitope-encoding nucleic acid sequences. The cassette encoding the one or more antigens can be 700 nucleotides or less and encode at least 2 distinct epitope-encoding nucleic acid sequences. The cassette encoding the one or more antigens can be 700 nucleotides or less and encode 3 distinct epitope-encoding nucleic acid sequences. The cassette encoding the one or more antigens can be 700 nucleotides or less and encode at least 3 distinct epitope-encoding nucleic acid sequences. The cassette encoding the one or more antigens can be 700 nucleotides or less and include 1-10, 1-5, 1, 2, 3, 4, 5, 6, 7, 8, 9, 10, or more antigens.

The cassette encoding the one or more antigens can be between 375-700 nucleotides in length. The cassette encoding the one or more antigens can be between 375-700 nucleotides in length and encode 2 distinct epitope-encoding nucleic acid sequences. The cassette encoding the one or more antigens can be between 375-700 nucleotides in length and encode at least 2 distinct epitope-encoding nucleic acid sequences (e.g., encode 2 distinct tumor derived nucleic acid sequences encoding an immunogenic polypeptide). The cassette encoding the one or more antigens can be between 375-700 nucleotides in length and encode 3 distinct epitope-encoding nucleic acid sequences. The cassette encoding the one or more antigens be between 375-700 nucleotides in length and encode at least 3 distinct epitope-encoding nucleic acid sequences. The cassette encoding the one or more antigens can be between 375-700 nucleotides in length and include 1-10, 1-5, 1, 2, 3, 4, 5, 6, 7, 8, 9, 10, or more antigens.

The cassette encoding the one or more antigens can be 600, 500, 400, 300, 200, or 100 nucleotides in length or less. The cassette encoding the one or more antigens can be 600, 500, 400, 300, 200, or 100 nucleotides in length or less and encode 2 distinct epitope-encoding nucleic acid sequences. The cassette encoding the one or more antigens can be 600, 500, 400, 300, 200, or 100 nucleotides in length or less and encode at least 2 distinct epitope-encoding nucleic acid sequences. The cassette encoding the one or more antigens can be 600, 500, 400, 300, 200, or 100 nucleotides in length or less and encode 3 distinct epitope-encoding nucleic acid sequences. The cassette encoding the one or more antigens can be 600, 500, 400, 300, 200, or 100 nucleotides in length or less and encode at least 3 distinct epitope-encoding nucleic acid sequences. The cassette encoding the one or more antigens can be 600, 500, 400, 300, 200, or 100 nucleotides in length or less and include 1-10, 1-5, 1, 2, 3, 4, 5, 6, 7, 8, 9, 10, or more antigens.

The cassette encoding the one or more antigens can be between 375-600, between 375-500, or between 375-400 nucleotides in length. The cassette encoding the one or more antigens can be between 375-600, between 375-500, or between 375-400 nucleotides in length and encode 2 distinct epitope-encoding nucleic acid sequences. The cassette encoding the one or more antigens can be between 375-600, between 375-500, or between 375-400 nucleotides in length and encode at least 2 distinct epitope-encoding nucleic acid sequences. The cassette encoding the one or more antigens can be between 375-600, between 375-500, or between 375-400 nucleotides in length and encode 3 distinct epitope-encoding nucleic acid sequences. The cassette encoding the one or more antigens can be between 375-600, between 375-500, or between 375-400 nucleotides in length and encode at least 3 distinct epitope-encoding nucleic acid sequences. The cassette encoding the one or more antigens can be between 375-600, between 375-500, or between 375-400 nucleotides in length and include 1-10, 1-5, 1, 2, 3, 4, 5, 6, 7, 8, 9, 10, or more antigens.

In some instances, an antigen or epitope in a cassette encoding additional antigens and/or epitopes may be an immunodominant epitope relative to the others encoded. Immunodominance, in general, is the skewing of an immune response towards only one or a few specific immunogenic peptides. Immunodominance can be assessed as part of an immune monitoring protocol. For example, immunodominance can be assessed through evaluating T cell and/or B cell responses to the encoded antigens.

Immunodominance can be assessed as the impact of an immunodominant antigen's presence on the immune response to one or more other antigens. For example, an immunodominant antigen and its respective immune response (e.g., an immunodominant MHC class I epitope) can reduce the immune response of another antigen relative to the immune response in the absence of the immunodominant antigen. This reduction can be such that the immune response in the presence of the immunodominant antigen is not considered a therapeutically effective response. For example, an MHC class I epitope would generally be considered immunodominant if T cell responses to other antigens are no longer considered therapeutically effective responses compared to responses elicited in the absence of the immunodominant MHC class I epitope. An immune response can also be reduced to below a limit of detection or near the limit of detection. relative to the response in the absence of the immunodominant antigen. For example, an MHC class I epitope would generally be considered immunodominant if T cell responses to other antigens are at or below the limit of detection compared to responses elicited in the absence of the immunodominant MHC class I epitope. In general, the assessment of immunodominance is between two antigens both capable of stimulating an immune response, e.g., between two T cell epitopes in a vaccine composition administered to a subject possessing a cognate MHC allele known or predicted to present each epitope, respectively. Immunodominance can be assessed through evaluating relative immune responses to other antigens in the presence and absence of the suspected immunodominant antigen.

Immunodominance can be assessed as a relative difference in the immune responses between two or more antigens. Immunodominance can refer to a 5-fold, 10-fold, 20-fold, 30-fold, 40-fold, or 50-fold immune response of a specific antigen relative to another antigen encoded in the same cassette. Immunodominance can refer to a 100-fold, 200-fold, 300-fold, 400-fold, or 500-fold immune response of a specific antigen relative to another antigen encoded in the same cassette. Immunodominance can refer to a 1000-fold, 2000-fold, 3000-fold, 4000-fold, or 5000-fold immune response of a specific antigen relative to another antigen encoded in the same cassette. Immunodominance can refer to a 10,000-fold immune response of a specific antigen relative to another antigen encoded in the same cassette.

In some instances, it may be desired to avoid vaccine compositions containing an immunodominant epitope. For example, it may be desired to avoid designing a vaccine cassette encoding an immunodominant epitope. Without wishing to be bound by theory, administering and/or encoding an immunodominant epitope together with additional epitope may reduce the immune response to the additional epitopes, including potentially ultimately reducing vaccine efficacy against the additional epitopes. As an illustrative non-limiting example, vaccine compositions including TP53-associated neoepitopes may have the immune response, e.g., a T cell response, skewed towards the TP53-associated neoepitope negatively impacting (e.g., reducing the immune response to where the immune response is not a therapeutically effective response and/or to below a limit of detection) the immune response to other antigens or epitopes in the vaccine composition (e.g., one or more CTA-associated neoepitopes in the vaccine composition). Accordingly, vaccine compositions can be designed to not contain an immunodominant epitope, such as designing a vaccine cassette (e.g., a (neo)antigen-encoding cassette) to not encode an immunodominant epitope. For example, the cassette does not encode an epitope that reduces an immune response to another epitope encoded in the cassette when administered in a vaccine composition to a subject relative to an immune response when the other epitope is administered in the absence of the immunodominant MHC class I epitope. In another example, the cassette does not encode an epitope that reduces an immune response to another epitope encoded in the cassette to below a limit of detection when administered in a vaccine composition to a subject relative to an immune response when the other epitope is administered in the absence of the immunodominant MHC class I epitope. In another example, the cassette does not encode an epitope that reduces an immune response to another epitope encoded in the cassette, wherein the immune response is not a therapeutically effective response, when administered in a vaccine composition to a subject relative to an immune response when the other epitope is administered in the absence of the immunodominant MHC class I epitope. In another example, the cassette does not encode an epitope that stimulates a 5-fold, 10-fold, 20-fold, 30-fold, 40-fold, or 50-fold or greater immune response relative to another epitope encoded in the same cassette in a vaccine composition administered to a subject, where each antigen is capable of stimulating an immune response in the subject. In another example, the cassette does not encode an epitope that stimulates a 100-fold, 200-fold, 300-fold, 400-fold, or 500-fold or greater immune response relative to another epitope encoded in the same cassette in a vaccine composition administered to a subject, where each antigen is capable of stimulating an immune response in the subject. In another example, the cassette does not encode an epitope that stimulates a 1000-fold, 2000-fold, 3000-fold, 4000-fold, or 5000-fold or greater immune response relative to another epitope encoded in the same cassette in a vaccine composition administered to a subject, where each antigen is capable of stimulating an immune response in the subject. In another example, the cassette does not encode an epitope that results in a 10,000-fold or greater immune response relative to another epitope encoded in the same cassette in a vaccine composition administered to a subject, where each antigen is capable of stimulating an immune response in the subject.

### V.B. Immune Modulators

Vectors described herein, such as C68 vectors described herein or alphavirus vectors described herein, can comprise a nucleic acid which encodes at least one antigen and the same or a separate vector can comprise a nucleic acid which encodes at least one immune modulator. An immune modulator can include a binding molecule (e.g., an antibody such as an scFv) which binds to and blocks the activity of an immune checkpoint molecule. An immune modulator can include a cytokine, such as IL-2, IL-7, IL-12 (including IL-12 p35, p40, p70, and/or p70-fusion constructs), IL-15, or IL-21. An immune modulator can include a modified cytokine (e.g., pegIL-2). Vectors can comprise an antigen cassette and one or more nucleic acid molecules encoding an immune modulator.

Illustrative immune checkpoint molecules that can be targeted for blocking or inhibition include, but are not limited to, CTLA-4, 4-1BB (CD137), 4-1BBL (CD137L), PDL1, PDL2, PD1, B7-H3, B7-H4, BTLA, HVEM, TIM3, GAL9, LAG3, TIM3, B7H3, B7H4, VISTA, KIR, 2B4 (belongs to the CD2 family of molecules and is expressed on all NK, γδ, and memory CD8+(αβ) T cells), CD160 (also referred to as BY55), and CGEN-15049. Immune checkpoint inhibitors include antibodies, or antigen binding fragments thereof, or other binding proteins, that bind to and block or inhibit the activity of one or more of CTLA-4, PDL1, PDL2, PD1, B7-H3, B7-H4, BTLA, HVEM, TIM3, GAL9, LAG3, TIM3, B7H3, B7H4, VISTA, KIR, 2B4, CD160, and CGEN-15049. Illustrative immune checkpoint inhibitors include Tremelimumab (CTLA-4 blocking antibody), anti-OX40, PD-L1 monoclonal Antibody (Anti-B7-H1; MEDI4736), ipilimumab, MK-3475 (PD-1 blocker), Nivolumamb (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody) and Yervoy/ipilimumab (anti-CTLA-4 checkpoint inhibitor). Antibody-encoding sequences can be engineered into vectors such as C68 using ordinary skill in the art. An exemplary method is described in Fang et al., Stable antibody expression at therapeutic levels using the 2A peptide. Nat Biotechnol. 2005 May; 23(5):584-90. Epub 2005 Apr. 17; herein incorporated by reference for all purposes.

### V.C. Additional Considerations for Vaccine Design and Manufacture

V.C.1. Determination of a Set of Peptides that Cover All Tumor Subclones

Truncal peptides, meaning those presented by all or most tumor subclones, can be prioritized for inclusion into a vaccine.⁵³ Optionally, if there are no truncal peptides predicted to be presented and immunogenic with high probability, or if the number of truncal peptides predicted to be presented and immunogenic with high probability is small enough that additional non-truncal peptides can be included in a vaccine, then further peptides can be prioritized by estimating the number and identity of tumor subclones and choosing peptides so as to maximize the number of tumor subclones covered by a vaccine.⁵⁴

### V.C.2. Antigen Prioritization

After all of the above antigen filters are applied, more candidate antigens may still be available for vaccine inclusion than a vaccine technology can support. Additionally, uncertainty about various aspects of the antigen analysis may remain and tradeoffs may exist between different properties of candidate vaccine antigens. Thus, in place of predetermined filters at each step of the selection process, an integrated multi-dimensional model can be considered that places candidate antigens in a space with at least the following axes and optimizes selection using an integrative approach.
1. Risk of auto-immunity or tolerance (risk of germline) (lower risk of auto-immunity is typically preferred)
2. Probability of sequencing artifact (lower probability of artifact is typically preferred)
3. Probability of immunogenicity (higher probability of immunogenicity is typically preferred)
4. Probability of presentation (higher probability of presentation is typically preferred)
5. Gene expression (higher expression is typically preferred)
6. Coverage of HLA genes (larger number of HLA molecules involved in the presentation of a set of antigens may lower the probability that a tumor will escape immune attack via downregulation or mutation of HLA molecules) 7. Coverage of HLA classes (covering both HLA-I and HLA-II may increase the probability of
therapeutic response and decrease the probability of tumor escape)

Additionally, optionally, antigens can be deprioritized (e.g., excluded) from the vaccination if they are predicted to be presented by HLA alleles lost or inactivated in either all or part of the patient's tumor or infected cell. HLA allele loss can occur by either somatic mutation, loss of heterozygosity, or homozygous deletion of the locus. Methods for detection of HLA allele somatic mutation are well known in the art, e.g. (Shukla et al., 2015). Methods for detection of somatic LOH and homozygous deletion (including for HLA locus) are likewise well described. (Carter et al., 2012; McGranahan et al., 2017; Van Loo et al., 2010). Antigens can also be deprioritized if mass-spectrometry data indicates a predicted antigen is not presented by a predicted HLA allele.

### V.D. Alphavirus

### V.D.1. Alphavirus Biology

Alphaviruses are members of the family Togaviridae, and are positive-sense single stranded RNA viruses. Members are typically classified as either Old World, such as Sindbis, Ross River, Mayaro, Chikungunya, and Semliki Forest viruses, or New World, such as eastern equine encephalitis, Aura, Fort Morgan, or Venezuelan equine encephalitis virus and its derivative strain TC-83 (Strauss Microbial Review 1994). A natural alphavirus genome is typically around 12 kb in length, the first two-thirds of which contain genes encoding non-structural proteins (nsPs) that form RNA replication complexes for self-replication of the viral genome, and the last third of which contains a subgenomic expression cassette encoding structural proteins for virion production (Frolov RNA 2001).

A model lifecycle of an alphavirus involves several distinct steps (Strauss Microbial Review 1994, Jose Future Microbiol 2009). Following virus attachment to a host cell, the virion fuses with membranes within endocytic compartments resulting in the eventual release of genomic RNA into the cytosol. The genomic RNA, which is in a plus-strand orientation and comprises a 5′ methylguanylate cap and 3′ polyA tail, is translated to produce non-structural proteins nsP1-4 that form the replication complex. Early in infection, the plus-strand is then replicated by the complex into a minus-stand template. In the current model, the replication complex is further processed as infection progresses, with the resulting processed complex switching to transcription of the minus-strand into both full-length positive-strand genomic RNA, as well as the 26S subgenomic positive-strand RNA containing the structural genes. Several conserved sequence elements (CSEs) of alphavirus have been identified to potentially play a role in the various RNA replication steps including; a complement of the 5′ UTR in the replication of plus-strand RNAs from a minus-strand template, a 51-nt CSE in the replication of minus-strand synthesis from the genomic template, a 24-nt CSE in the junction region between the nsPs and the 26S RNA in the transcription of the subgenomic RNA from the minus-strand, and a 3′ 19-nt CSE in minus-strand synthesis from the plus-strand template.

Following the replication of the various RNA species, virus particles are then typically assembled in the natural lifecycle of the virus. The 26S RNA is translated and the resulting proteins further processed to produce the structural proteins including capsid protein, glycoproteins E1 and E2, and two small polypeptides E3 and 6K (Strauss 1994). Encapsidation of viral RNA occurs, with capsid proteins normally specific for only genomic RNA being packaged, followed by virion assembly and budding at the membrane surface.

### V.D.2. Alphavirus as a Delivery Vector

Alphaviruses (including alphavirus sequences, features, and other elements) can be used to generate alphavirus-based delivery vectors (also be referred to as alphavirus vectors, alphavirus viral vectors, alphavirus vaccine vectors, self-replicating RNA (srRNA) vectors, or self-amplifying RNA (samRNA) vectors). Alphaviruses have previously been engineered for use as expression vector systems (Pushko 1997, Rheme 2004). Alphaviruses offer several advantages, particularly in a vaccine setting where heterologous antigen expression can be desired. Due to its ability to self-replicate in the host cytosol, alphavirus vectors are generally able to produce high copy numbers of the expression cassette within a cell resulting in a high level of heterologous antigen production. Additionally, the vectors are generally transient, resulting in improved biosafety as well as reduced induction of immunological tolerance to the vector. The public, in general, also lacks pre-existing immunity to alphavirus vectors as compared to other standard viral vectors, such as human adenovirus. Alphavirus based vectors also generally result in cytotoxic responses to infected cells. Cytotoxicity, to a certain degree, can be important in a vaccine setting to properly stimulate an immune response to the heterologous antigen expressed. However, the degree of desired cytotoxicity can be a balancing act, and thus several attenuated alphaviruses have been developed, including the TC-83 strain of VEE. Thus, an example of an antigen expression vector described herein can utilize an alphavirus backbone that allows for a high level of antigen expression, stimulates a robust immune response to antigen, does not stimulate an immune response to the vector itself, and can be used in a safe manner. Furthermore, the antigen expression cassette can be designed to stimulate different levels of an immune response through optimization of which alphavirus sequences the vector uses, including, but not limited to, sequences derived from VEE or its attenuated derivative TC-83.

Several expression vector design strategies have been engineered using alphavirus sequences (Pushko 1997). In one strategy, a alphavirus vector design includes inserting a second copy of the 26S promoter sequence elements downstream of the structural protein genes, followed by a heterologous gene (Frolov 1993). Thus, in addition to the natural non-structural and structural proteins, an additional subgenomic RNA is produced that expresses the heterologous protein. In this system, all the elements for production of infectious virions are present and, therefore, repeated rounds of infection of the expression vector in non-infected cells can occur.

Another expression vector design makes use of helper virus systems (Pushko 1997). In this strategy, the structural proteins are replaced by a heterologous gene. Thus, following self-replication of viral RNA mediated by still intact non-structural genes, the 26S subgenomic RNA provides for expression of the heterologous protein. Traditionally, additional vectors that expresses the structural proteins are then supplied in trans, such as by co-transfection of a cell line, to produce infectious virus. A system is described in detail in U.S. Pat. No. 8,093,021, which is herein incorporated by reference in its entirety, for all purposes. The helper vector system provides the benefit of limiting the possibility of forming infectious particles and, therefore, improves biosafety. In addition, the helper vector system reduces the total vector length, potentially improving the replication and expression efficiency. Thus, an example of an antigen expression vector described herein can utilize an alphavirus backbone wherein the structural proteins are replaced by an antigen cassette, the resulting vector both reducing biosafety concerns, while at the same time promoting efficient expression due to the reduction in overall expression vector size.

### V.D.3. Alphavirus Production In Vitro

Alphavirus delivery vectors are generally positive-sense RNA polynucleotides. A convenient technique well-known in the art for RNA production is in vitro transcription IVT. In this technique, a DNA template of the desired vector is first produced by techniques well-known to those in the art, including standard molecular biology techniques such as cloning, restriction digestion, ligation, gene synthesis (e.g., chemical and/or enzymatic synthesis), and polymerase chain reaction (PCR). The DNA template contains a RNA polymerase promoter at the 5′ end of the sequence desired to be transcribed into RNA. Promoters include, but are not limited to, bacteriophage polymerase promoters such as T3, T7, or SP6. The DNA template is then incubated with the appropriate RNA polymerase enzyme, buffer agents, and nucleotides (NTPs). The resulting RNA polynucleotide can optionally be further modified including, but limited to, addition of a 5′ cap structure such as 7-methylguanosine or a related structure, and optionally modifying the 3′ end to include a polyadenylate (polyA) tail. The RNA can then be purified using techniques well-known in the field, such as phenol-chloroform extraction or column purification (e.g., chromatography-based purification).

### V.D.4. Delivery Via Lipid Nanoparticle

An important aspect to consider in vaccine vector design is immunity against the vector itself (Riley 2017). This may be in the form of preexisting immunity to the vector itself, such as with certain human adenovirus systems, or in the form of developing immunity to the vector following administration of the vaccine. The latter is an important consideration if multiple administrations of the same vaccine are performed, such as separate priming and boosting doses, or if the same vaccine vector system is to be used to deliver different antigen cassettes.

In the case of alphavirus vectors, the standard delivery method is the previously discussed helper virus system that provides capsid, E1, and E2 proteins in trans to produce infectious viral particles. However, it is important to note that the E1 and E2 proteins are often major targets of neutralizing antibodies (Strauss 1994). Thus, the efficacy of using alphavirus vectors to deliver antigens of interest to target cells may be reduced if infectious particles are targeted by neutralizing antibodies.

An alternative to viral particle mediated gene delivery is the use of nanomaterials to deliver expression vectors (Riley 2017). Nanomaterial vehicles, importantly, can be made of non-immunogenic materials and generally avoid stimulating immunity to the delivery vector itself. These materials can include, but are not limited to, lipids, inorganic nanomaterials, and other polymeric materials. Lipids can be cationic, anionic, or neutral. The materials can be synthetic or naturally derived, and in some instances biodegradable. Lipids can include fats, cholesterol, phospholipids, lipid conjugates including, but not limited to, polyethyleneglycol (PEG) conjugates (PEGylated lipids), waxes, oils, glycerides, and fat soluble vitamins.

Lipid nanoparticles (LNPs) are an attractive delivery system due to the amphiphilic nature of lipids enabling formation of membranes and vesicle like structures (Riley 2017). In general, these vesicles deliver the expression vector by absorbing into the membrane of target cells and releasing nucleic acid into the cytosol. In addition, LNPs can be further modified or functionalized to facilitate targeting of specific cell types. Another consideration in LNP design is the balance between targeting efficiency and cytotoxicity. Lipid compositions generally include defined mixtures of cationic, neutral, anionic, and amphipathic lipids. In some instances, specific lipids are included to prevent LNP aggregation, prevent lipid oxidation, or provide functional chemical groups that facilitate attachment of additional moieties. Lipid composition can influence overall LNP size and stability. In an example, the lipid composition comprises dilinoleylmethyl-4-dimethylaminobutyrate (MC3) or MC3-like molecules. MC3 and MC3-like lipid compositions can be formulated to include one or more other lipids, such as a PEG or PEG-conjugated lipid, a sterol, or neutral lipids.

Nucleic-acid vectors, such as expression vectors, exposed directly to serum can have several undesirable consequences, including degradation of the nucleic acid by serum nucleases or off-target stimulation of the immune system by the free nucleic acids. Therefore, encapsulation of the alphavirus vector can be used to avoid degradation, while also avoiding potential off-target affects. In certain examples, an alphavirus vector is fully encapsulated within the delivery vehicle, such as within the aqueous interior of an LNP. Encapsulation of the alphavirus vector within an LNP can be carried out by techniques well-known to those skilled in the art, such as microfluidic mixing and droplet generation carried out on a microfluidic droplet generating device. Such devices include, but are not limited to, standard T-junction devices or flow-focusing devices. In an example, the desired lipid formulation, such as MC3 or MC3-like containing compositions, is provided to the droplet generating device in parallel with the alphavirus delivery vector and other desired agents, such that the delivery vector and desired agents are fully encapsulated within the interior of the MC3 or MC3-like based LNP. In an example, the droplet generating device can control the size range and size distribution of the LNPs produced. For example, the LNP can have a size ranging from 1 to 1000 nanometers in diameter, e.g., 1, 10, 50, 100, 500, or 1000 nanometers. Following droplet generation, the delivery vehicles encapsulating the expression vectors can be further treated or modified to prepare them for administration.

### V.E. Chimpanzee Adenovirus (ChAd)

V.E.1. Viral Delivery with Chimpanzee Adenovirus

Vaccine compositions for delivery of one or more antigens (e.g., via an antigen cassette) can be created by providing adenovirus nucleotide sequences of chimpanzee origin, a variety of novel vectors, and cell lines expressing chimpanzee adenovirus genes. A nucleotide sequence of a chimpanzee C68 adenovirus (also referred to herein as ChAdV68) can be used in a vaccine composition for antigen delivery (See SEQ ID NO: 1). Use of C68 adenovirus derived vectors is described in further detail in U.S. Pat. No. 6,083,716, which is herein incorporated by reference in its entirety, for all purposes. ChAdV68-based vectors and delivery systems are described in detail in US App. Pub. No. US20200197500A1 and international patent application publication WO2020243719A1, each of which is herein incorporated by reference for all purposes.

In a further aspect, provided herein is a recombinant adenovirus comprising the DNA sequence of a chimpanzee adenovirus such as C68 and an antigen cassette operatively linked to regulatory sequences directing its expression. The recombinant virus is capable of infecting a mammalian, preferably a human, cell and capable of expressing the antigen cassette product in the cell. In this vector, the native chimpanzee E1 gene, and/or E3 gene, and/or E4 gene can be deleted. An antigen cassette can be inserted into any of these sites of gene deletion. The antigen cassette can include an antigen against which a primed immune response is desired.

In another aspect, provided herein is a mammalian cell infected with a chimpanzee adenovirus such as C68.

In still a further aspect, a novel mammalian cell line is provided which expresses a chimpanzee adenovirus gene (e.g., from C68) or functional fragment thereof.

In still a further aspect, provided herein is a method for delivering an antigen cassette into a mammalian cell comprising the step of introducing into the cell an effective amount of a chimpanzee adenovirus, such as C68, that has been engineered to express the antigen cassette.

Still another aspect provides a method for stimulating an immune response in a mammalian host to treat cancer. The method can comprise the step of administering to the host an effective amount of a recombinant chimpanzee adenovirus, such as C68, comprising an antigen cassette that encodes one or more antigens from the tumor against which the immune response is targeted.

Still another aspect provides a method for stimulating an immune response in a mammalian host to treat or prevent a disease in a subject, such as an infectious disease. The method can comprise the step of administering to the host an effective amount of a recombinant chimpanzee adenovirus, such as C68, comprising an antigen cassette that encodes one or more antigens, such as from the infectious disease against which the immune response is targeted.

Also disclosed is a non-simian mammalian cell that expresses a chimpanzee adenovirus gene obtained from the sequence of SEQ ID NO: 1. The gene can be selected from the group consisting of the adenovirus E1A, E1B, E2A, E2B, E3, E4, L1, L2, L3, L4 and L5 of SEQ ID NO: 1.

Also disclosed is a nucleic acid molecule comprising a chimpanzee adenovirus DNA sequence comprising a gene obtained from the sequence of SEQ ID NO: 1. The gene can be selected from the group consisting of said chimpanzee adenovirus E1A, E1B, E2A, E2B, E3, E4, L1, L2, L3, L4 and L5 genes of SEQ ID NO: 1. In some aspects the nucleic acid molecule comprises SEQ ID NO: 1. In some aspects the nucleic acid molecule comprises the sequence of SEQ ID NO: 1, lacking at least one gene selected from the group consisting of E1A, E1B, E2A, E2B, E3, E4, L1, L2, L3, L4 and L5 genes of SEQ ID NO: 1.

Also disclosed is a vector comprising a chimpanzee adenovirus DNA sequence obtained from SEQ ID NO: 1 and an antigen cassette operatively linked to one or more regulatory sequences which direct expression of the cassette in a heterologous host cell, optionally wherein the chimpanzee adenovirus DNA sequence comprises at least the cis-elements necessary for replication and virion encapsidation, the cis-elements flanking the antigen cassette and regulatory sequences. In some aspects, the chimpanzee adenovirus DNA sequence comprises a gene selected from the group consisting of E1A, E1B, E2A, E2B, E3, E4, L1, L2, L3, L4 and L5 gene sequences of SEQ ID NO: 1. In some aspects the vector can lack the E1A and/or E1B gene.

Also disclosed herein is a adenovirus vector comprising: a partially deleted E4 gene comprising a deleted or partially-deleted E4orf2 region and a deleted or partially-deleted E4orf3 region, and optionally a deleted or partially-deleted E4orf4 region. The partially deleted E4 can comprise an E4 deletion of at least nucleotides 34,916 to 35,642 of the sequence shown in SEQ ID NO:1, and wherein the vector comprises at least nucleotides 2 to 36,518 of the sequence set forth in SEQ ID NO:1. The partially deleted E4 can comprise an E4 deletion of at least a partial deletion of nucleotides 34,916 to 34,942 of the sequence shown in SEQ ID NO:1, at least a partial deletion of nucleotides 34,952 to 35,305 of the sequence shown in SEQ ID NO:1, and at least a partial deletion of nucleotides 35,302 to 35,642 of the sequence shown in SEQ ID NO:1, and wherein the vector comprises at least nucleotides 2 to 36,518 of the sequence set forth in SEQ ID NO:1 The partially deleted E4 can comprise an E4 deletion of at least nucleotides 34,980 to 36,516 of the sequence shown in SEQ ID NO:1, and wherein the vector comprises at least nucleotides 2 to 36,518 of the sequence set forth in SEQ ID NO:1. The partially deleted E4 can comprise an E4 deletion of at least nucleotides 34,979 to 35,642 of the sequence shown in SEQ ID NO:1, and wherein the vector comprises at least nucleotides 2 to 36,518 of the sequence set forth in SEQ ID NO:1. The partially deleted E4 can comprise an E4 deletion of at least a partial deletion of E4Orf2, a fully deleted E4Orf3, and at least a partial deletion of E4Orf4. The partially deleted E4 can comprise an E4 deletion of at least a partial deletion of E4Orf2, at least a partial deletion of E4Orf3, and at least a partial deletion of E4Orf4. The partially deleted E4 can comprise an E4 deletion of at least a partial deletion of E4Orf1, a fully deleted E4Orf2, and at least a partial deletion of E4Orf3. The partially deleted E4 can comprise an E4 deletion of at least a partial deletion of E4Orf2 and at least a partial deletion of E4Orf3. The partially deleted E4 can comprise an E4 deletion between the start site of E4Orf1 to the start site of E4Orf5. The partially deleted E4 can be an E4 deletion adjacent to the start site of E4Orf1. The partially deleted E4 can be an E4 deletion adjacent to the start site of E4Orf2. The partially deleted E4 can be an E4 deletion adjacent to the start site of E4Orf3. The partially deleted E4 can be an E4 deletion adjacent to the start site of E4Orf4. The E4 deletion can be at least 50, at least 100, at least 200, at least 300, at least 400, at least 500, at least 600, at least 700, at least 800, at least 900, at least 1000, at least 1100, at least 1200, at least 1300, at least 1400, at least 1500, at least 1600, at least 1700, at least 1800, at least 1900, or at least 2000 nucleotides. The E4 deletion can be at least 700 nucleotides. The E4 deletion can be at least 1500 nucleotides. The E4 deletion can be 50 or less, 100 or less, 200 or less, 300 or less, 400 or less, 500 or less, 600 or less, 700 or less, 800 or less, 900 or less, 1000 or less, 1100 or less, 1200 or less, 1300 or less, 1400 or less, 1500 or less, 1600 or less, 1700 or less, 1800 or less, 1900 or less, or 2000 or less nucleotides. The E4 deletion can be 750 nucleotides or less. The E4 deletion can be at least 1550 nucleotides or less.

The partially deleted E4 gene can be the E4 gene sequence shown in SEQ ID NO:1 that lacks at least nucleotides 34,916 to 35,642 of the sequence shown in SEQ ID NO:1. The partially deleted E4 gene can be the E4 gene sequence shown in SEQ ID NO:1 that lacks the E4 gene sequence shown in SEQ ID NO:1 and that lacks at least nucleotides 34,916 to 34,942, nucleotides 34,952 to 35,305 of the sequence shown in SEQ ID NO:1, and nucleotides 35,302 to 35,642 of the sequence shown in SEQ ID NO:1. The partially deleted E4 gene can be the E4 gene sequence shown in SEQ ID NO:1 and that lacks at least nucleotides 34,980 to 36,516 of the sequence shown in SEQ ID NO:1. The partially deleted E4 gene can be the E4 gene sequence shown in SEQ ID NO:1 and that lacks at least nucleotides 34,979 to 35,642 of the sequence shown in SEQ ID NO:1. The adenovirus vector having the partially deleted E4 gene can have a cassette, wherein the cassette comprises at least one payload nucleic acid sequence, and wherein the cassette comprises at least one promoter sequence operably linked to the at least one payload nucleic acid sequence. The adenovirus vector having the partially deleted E4 gene can have one or more genes or regulatory sequences of the ChAdV68 sequence shown in SEQ ID NO: 1, optionally wherein the one or more genes or regulatory sequences comprise at least one of the chimpanzee adenovirus inverted terminal repeat (ITR), E1A, E1B, E2A, E2B, E3, E4, L1, L2, L3, L4, and L5 genes of the sequence shown in SEQ ID NO: 1. The adenovirus vector having the partially deleted E4 gene can have nucleotides 2 to 34,916 of the sequence shown in SEQ ID NO:1, wherein the partially deleted E4 gene is 3′ of the nucleotides 2 to 34,916, and optionally the nucleotides 2 to 34,916 additionally lack nucleotides 577 to 3403 of the sequence shown in SEQ ID NO:1 corresponding to an E1 deletion and/or lack nucleotides 27,125 to 31,825 of the sequence shown in SEQ ID NO:1 corresponding to an E3 deletion. The adenovirus vector having the partially deleted E4 gene can have nucleotides 35,643 to 36,518 of the sequence shown in SEQ ID NO:1, and wherein the partially deleted E4 gene is 5′ of the nucleotides 35,643 to 36,518. The adenovirus vector having the partially deleted E4 gene can have nucleotides 2 to 34,916 of the sequence shown in SEQ ID NO:1, wherein the partially deleted E4 gene is 3′ of the nucleotides 2 to 34,916, the nucleotides 2 to 34,916 additionally lack nucleotides 577 to 3403 of the sequence shown in SEQ ID NO:1 corresponding to an E1 deletion and lack nucleotides 27,125 to 31,825 of the sequence shown in SEQ ID NO:1 corresponding to an E3 deletion. The adenovirus vector having the partially deleted E4 gene can have nucleotides 2 to 34,916 of the sequence shown in SEQ ID NO:1, wherein the partially deleted E4 gene is 3′ of the nucleotides 2 to 34,916, the nucleotides 2 to 34,916 additionally lack nucleotides 577 to 3403 of the sequence shown in SEQ ID NO:1 corresponding to an E1 deletion and lack nucleotides 27,125 to 31,825 of the sequence shown in SEQ ID NO:1 corresponding to an E3 deletion, and have nucleotides 35,643 to 36,518 of the sequence shown in SEQ ID NO:1, and wherein the partially deleted E4 gene is 5′ of the nucleotides 35,643 to 36,518.

The partially deleted E4 gene can be the E4 gene sequence shown in SEQ ID NO:1 that lacks at least nucleotides 34,916 to 35,642 of the sequence shown in SEQ ID NO:1, nucleotides 2 to 34,916 of the sequence shown in SEQ ID NO:1, wherein the partially deleted E4 gene is 3′ of the nucleotides 2 to 34,916, the nucleotides 2 to 34,916 additionally lack nucleotides 577 to 3403 of the sequence shown in SEQ ID NO:1 corresponding to an E1 deletion and lack nucleotides 27,125 to 31,825 of the sequence shown in SEQ ID NO:1 corresponding to an E3 deletion, and have nucleotides 35,643 to 36,518 of the sequence shown in SEQ ID NO:1, and wherein the partially deleted E4 gene is 5′ of the nucleotides 35,643 to 36,518.

Also disclosed herein is a host cell transfected with a vector disclosed herein such as a C68 vector engineered to expression an antigen cassette. Also disclosed herein is a human cell that expresses a selected gene introduced therein through introduction of a vector disclosed herein into the cell.

Also disclosed herein is a method for delivering an antigen cassette to a mammalian cell comprising introducing into said cell an effective amount of a vector disclosed herein such as a C68 vector engineered to expression the antigen cassette.

Also disclosed herein is a method for producing an antigen comprising introducing a vector disclosed herein into a mammalian cell, culturing the cell under suitable conditions and producing the antigen.

### V.E.2. E1-Expressing Complementation Cell Lines

To generate recombinant chimpanzee adenoviruses (Ad) deleted in any of the genes described herein, the function of the deleted gene region, if essential to the replication and infectivity of the virus, can be supplied to the recombinant virus by a helper virus or cell line, i.e., a complementation or packaging cell line. For example, to generate a replication-defective chimpanzee adenovirus vector, a cell line can be used which expresses the E1 gene products of the human or chimpanzee adenovirus; such a cell line can include HEK293 or variants thereof. The protocol for the generation of the cell lines expressing the chimpanzee E1 gene products (Examples 3 and 4 of U.S. Pat. No. 6,083,716) can be followed to generate a cell line which expresses any selected chimpanzee adenovirus gene.

An AAV augmentation assay can be used to identify a chimpanzee adenovirus E1-expressing cell line. This assay is useful to identify E1 function in cell lines made by using the E1 genes of other uncharacterized adenoviruses, e.g., from other species. That assay is described in Example 4B of U.S. Pat. No. 6,083,716.

A selected chimpanzee adenovirus gene, e.g., E1, can be under the transcriptional control of a promoter for expression in a selected parent cell line. Inducible or constitutive promoters can be employed for this purpose. Among inducible promoters are included the sheep metallothionine promoter, inducible by zinc, or the mouse mammary tumor virus (MMTV) promoter, inducible by a glucocorticoid, particularly, dexamethasone. Other inducible promoters, such as those identified in International patent application publication WO95/13392, incorporated by reference herein can also be used in the production of packaging cell lines. Constitutive promoters in control of the expression of the chimpanzee adenovirus gene can be employed also.

A parent cell can be selected for the generation of a novel cell line expressing any desired C68 gene. Without limitation, such a parent cell line can be HeLa [ATCC Accession No. CCL 2], A549 [ATCC Accession No. CCL 185], KB [CCL 17], Detroit [e.g., Detroit 510, CCL 72] and WI-38 [CCL 75] cells. Other suitable parent cell lines can be obtained from other sources. Parent cell lines can include CHO, HEK293 or variants thereof, 911, HeLa, A549, LP-293, PER.C6, or AE1-2a.

An E1-expressing cell line can be useful in the generation of recombinant chimpanzee adenovirus E1 deleted vectors. Cell lines constructed using essentially the same procedures that express one or more other chimpanzee adenoviral gene products are useful in the generation of recombinant chimpanzee adenovirus vectors deleted in the genes that encode those products. Further, cell lines which express other human Ad E1 gene products are also useful in generating chimpanzee recombinant Ads.

### V.E.3. Recombinant Viral Particles as Vectors

The compositions disclosed herein can comprise viral vectors, that deliver at least one antigen to cells. Such vectors comprise a chimpanzee adenovirus DNA sequence such as C68 and an antigen cassette operatively linked to regulatory sequences which direct expression of the cassette. The C68 vector is capable of expressing the cassette in an infected mammalian cell. The C68 vector can be functionally deleted in one or more viral genes. An antigen cassette comprises at least one antigen under the control of one or more regulatory sequences such as a promoter. Optional helper viruses and/or packaging cell lines can supply to the chimpanzee viral vector any necessary products of deleted adenoviral genes.

The term “functionally deleted” means that a sufficient amount of the gene region is removed or otherwise altered, e.g., by mutation or modification, so that the gene region is no longer capable of producing one or more functional products of gene expression. Mutations or modifications that can result in functional deletions include, but are not limited to, nonsense mutations such as introduction of premature stop codons and removal of canonical and non-canonical start codons, mutations that alter mRNA splicing or other transcriptional processing, or combinations thereof. If desired, the entire gene region can be removed.

Modifications of the nucleic acid sequences forming the vectors disclosed herein, including sequence deletions, insertions, and other mutations may be generated using standard molecular biological techniques and are within the scope of this invention.

### V.E.4. Construction of The Viral Plasmid Vector

The chimpanzee adenovirus C68 vectors useful in this invention include recombinant, defective adenoviruses, that is, chimpanzee adenovirus sequences functionally deleted in the E1a or E1b genes, and optionally bearing other mutations, e.g., temperature-sensitive mutations or deletions in other genes. It is anticipated that these chimpanzee sequences are also useful in forming hybrid vectors from other adenovirus and/or adeno-associated virus sequences. Homologous adenovirus vectors prepared from human adenoviruses are described in the published literature [see, for example, Kozarsky I and II, cited above, and references cited therein, U.S. Pat. No. 5,240,846].

In the construction of useful chimpanzee adenovirus C68 vectors for delivery of an antigen cassette to a human (or other mammalian) cell, a range of adenovirus nucleic acid sequences can be employed in the vectors. A vector comprising minimal chimpanzee C68 adenovirus sequences can be used in conjunction with a helper virus to produce an infectious recombinant virus particle. The helper virus provides essential gene products required for viral infectivity and propagation of the minimal chimpanzee adenoviral vector. When only one or more selected deletions of chimpanzee adenovirus genes are made in an otherwise functional viral vector, the deleted gene products can be supplied in the viral vector production process by propagating the virus in a selected packaging cell line that provides the deleted gene functions in trans.

### V.E.5. Recombinant Minimal Adenovirus

A minimal chimpanzee Ad C68 virus is a viral particle containing just the adenovirus cis-elements necessary for replication and virion encapsidation. That is, the vector contains the cis-acting 5′ and 3′ inverted terminal repeat (ITR) sequences of the adenoviruses (which function as origins of replication) and the native 5′ packaging/enhancer domains (that contain sequences necessary for packaging linear Ad genomes and enhancer elements for the E1 promoter). See, for example, the techniques described for preparation of a “minimal” human Ad vector in International Patent Application WO96/13597 and incorporated herein by reference.

### V.E.6. Other Defective Adenoviruses

Recombinant, replication-deficient adenoviruses can also contain more than the minimal chimpanzee adenovirus sequences. These other Ad vectors can be characterized by deletions of various portions of gene regions of the virus, and infectious virus particles formed by the optional use of helper viruses and/or packaging cell lines.

As one example, suitable vectors may be formed by deleting all or a sufficient portion of the C68 adenoviral immediate early gene E1a and delayed early gene E1b, so as to eliminate their normal biological functions. Replication-defective E1-deleted viruses are capable of replicating and producing infectious virus when grown on a chimpanzee adenovirus-transformed, complementation cell line containing functional adenovirus E1a and E1b genes which provide the corresponding gene products in trans. Based on the homologies to known adenovirus sequences, it is anticipated that, as is true for the human recombinant E1-deleted adenoviruses of the art, the resulting recombinant chimpanzee adenovirus is capable of infecting many cell types and can express antigen(s), but cannot replicate in most cells that do not carry the chimpanzee E1 region DNA unless the cell is infected at a very high multiplicity of infection.

As another example, all or a portion of the C68 adenovirus delayed early gene E3 can be eliminated from the chimpanzee adenovirus sequence which forms a part of the recombinant virus.

Chimpanzee adenovirus C68 vectors can also be constructed having a deletion of the E4 gene. Still another vector can contain a deletion in the delayed early gene E2a.

Deletions can also be made in any of the late genes L1 through L5 of the chimpanzee C68 adenovirus genome. Similarly, deletions in the intermediate genes IX and IVa2 can be useful for some purposes. Other deletions may be made in the other structural or non-structural adenovirus genes.

The above discussed deletions can be used individually, i.e., an adenovirus sequence can contain deletions of E1 only. Alternatively, deletions of entire genes or portions thereof effective to destroy or reduce their biological activity can be used in any combination. For example, in one exemplary vector, the adenovirus C68 sequence can have deletions of the E1 genes and the E4 gene, or of the E1, E2a and E3 genes, or of the E1 and E3 genes, or of E1, E2a and E4 genes, with or without deletion of E3, and so on. As discussed above, such deletions can be used in combination with other mutations, such as temperature-sensitive mutations, to achieve a desired result.

The cassette comprising antigen(s) be inserted optionally into any deleted region of the chimpanzee C68 Ad virus. Alternatively, the cassette can be inserted into an existing gene region to disrupt the function of that region, if desired.

### V.E.7. Helper Viruses

Depending upon the chimpanzee adenovirus gene content of the viral vectors employed to carry the antigen cassette, a helper adenovirus or non-replicating virus fragment can be used to provide sufficient chimpanzee adenovirus gene sequences to produce an infective recombinant viral particle containing the cassette.

Useful helper viruses contain selected adenovirus gene sequences not present in the adenovirus vector construct and/or not expressed by the packaging cell line in which the vector is transfected. A helper virus can be replication-defective and contain a variety of adenovirus genes in addition to the sequences described above. The helper virus can be used in combination with the E1-expressing cell lines described herein.

For C68, the “helper” virus can be a fragment formed by clipping the C terminal end of the C68 genome with SspI, which removes about 1300 bp from the left end of the virus. This clipped virus is then co-transfected into an E1-expressing cell line with the plasmid DNA, thereby forming the recombinant virus by homologous recombination with the C68 sequences in the plasmid.

Helper viruses can also be formed into poly-cation conjugates as described in Wu et al, J. Biol. Chem., 264:16985-16987 (1989); K. J. Fisher and J. M. Wilson, Biochem. J., 299:49 (Apr. 1, 1994). Helper virus can optionally contain a reporter gene. A number of such reporter genes are known to the art. The presence of a reporter gene on the helper virus which is different from the antigen cassette on the adenovirus vector allows both the Ad vector and the helper virus to be independently monitored. This second reporter is used to enable separation between the resulting recombinant virus and the helper virus upon purification.

### V.E.8. Assembly of Viral Particle and Infection of a Cell Line

Assembly of the selected DNA sequences of the adenovirus, the antigen cassette, and other vector elements into various intermediate plasmids and shuttle vectors, and the use of the plasmids and vectors to produce a recombinant viral particle can all be achieved using conventional techniques. Such techniques include conventional cloning techniques of cDNA, in vitro recombination techniques (e.g., Gibson assembly), use of overlapping oligonucleotide sequences of the adenovirus genomes, polymerase chain reaction, and any suitable method which provides the desired nucleotide sequence. Standard transfection and co-transfection techniques are employed, e.g., CaPO4 precipitation techniques or liposome-mediated transfection methods such as lipofectamine. Other conventional methods employed include homologous recombination of the viral genomes, plaquing of viruses in agar overlay, methods of measuring signal generation, and the like.

For example, following the construction and assembly of the desired antigen cassette-containing viral vector, the vector can be transfected in vitro in the presence of a helper virus into the packaging cell line. Homologous recombination occurs between the helper and the vector sequences, which permits the adenovirus-antigen sequences in the vector to be replicated and packaged into virion capsids, resulting in the recombinant viral vector particles.

The resulting recombinant chimpanzee C68 adenoviruses are useful in transferring an antigen cassette to a selected cell. In in vivo experiments with the recombinant virus grown in the packaging cell lines, the E1-deleted recombinant chimpanzee adenovirus demonstrates utility in transferring a cassette to a non-chimpanzee, preferably a human, cell.

### V.E.9. Use of the Recombinant Virus Vectors

The resulting recombinant chimpanzee C68 adenovirus containing the antigen cassette (produced by cooperation of the adenovirus vector and helper virus or adenoviral vector and packaging cell line, as described above) thus provides an efficient gene transfer vehicle which can deliver antigen(s) to a subject in vivo or ex vivo.

The above-described recombinant vectors are administered to humans according to published methods for gene therapy. A chimpanzee viral vector bearing an antigen cassette can be administered to a patient, preferably suspended in a biologically compatible solution or pharmaceutically acceptable delivery vehicle. A suitable vehicle includes sterile saline. Other aqueous and non-aqueous isotonic sterile injection solutions and aqueous and non-aqueous sterile suspensions known to be pharmaceutically acceptable carriers and well known to those of skill in the art may be employed for this purpose.

The chimpanzee adenoviral vectors are administered in sufficient amounts to transduce the human cells and to provide sufficient levels of antigen transfer and expression to provide a therapeutic benefit without undue adverse or with medically acceptable physiological effects, which can be determined by those skilled in the medical arts. Conventional and pharmaceutically acceptable routes of administration include, but are not limited to, direct delivery to the liver, intranasal, intravenous, intramuscular, subcutaneous, intradermal, oral and other parental routes of administration. Routes of administration may be combined, if desired.

Dosages of the viral vector will depend primarily on factors such as the condition being treated, the age, weight and health of the patient, and may thus vary among patients. The dosage will be adjusted to balance the therapeutic benefit against any side effects and such dosages may vary depending upon the therapeutic application for which the recombinant vector is employed. The levels of expression of antigen(s) can be monitored to determine the frequency of dosage administration.

Recombinant, replication defective adenoviruses can be administered in a “pharmaceutically effective amount”, that is, an amount of recombinant adenovirus that is effective in a route of administration to transfect the desired cells and provide sufficient levels of expression of the selected gene to provide a vaccinal benefit, i.e., some measurable level of protective immunity. C68 vectors comprising an antigen cassette can be co-administered with adjuvant. Adjuvant can be separate from the vector (e.g., alum) or encoded within the vector, in particular if the adjuvant is a protein. Adjuvants are well known in the art.

Conventional and pharmaceutically acceptable routes of administration include, but are not limited to, intranasal, intramuscular, intratracheal, subcutaneous, intradermal, rectal, oral and other parental routes of administration. Routes of administration may be combined, if desired, or adjusted depending upon the immunogen or the disease. For example, in prophylaxis of rabies, the subcutaneous, intratracheal and intranasal routes are preferred. The route of administration primarily will depend on the nature of the disease being treated.

The levels of immunity to antigen(s) can be monitored to determine the need, if any, for boosters. Following an assessment of antibody titers in the serum, for example, optional booster immunizations may be desired

### VI. Therapeutic and Manufacturing Methods

Also provided is a method of inducing a tumor specific immune response in a subject, vaccinating against a tumor, treating and/or alleviating a symptom of cancer in a subject by administering to the subject one or more antigens such as a plurality of antigens identified using methods disclosed herein.

In some aspects, a subject has been diagnosed with cancer or is at risk of developing cancer. A subject can be a human, dog, cat, horse or any animal in which a tumor specific immune response is desired. A tumor can be any solid tumor such as breast, ovarian, prostate, lung, kidney, gastric, colon, testicular, head and neck, pancreas, brain, melanoma, and other tumors of tissue organs and hematological tumors, such as lymphomas and leukemias, including acute myelogenous leukemia, chronic myelogenous leukemia, chronic lymphocytic leukemia, T cell lymphocytic leukemia, and B cell lymphomas. A cancer can be non-small cell lung cancer (NSCLC).

An antigen can be administered in an amount sufficient to stimulate a CTL response. An antigen can be administered in an amount sufficient to stimulate a T cell response. An antigen can be administered in an amount sufficient to stimulate a B cell response. An antigen can be administered in an amount sufficient to stimulate both a T cell response and a B cell response.

An antigen can be administered alone or in combination with other therapeutic agents. Therapeutic agents can include those that target an infectious disease organism, such as an anti-viral or antibiotic agent.

In addition, a subject can be further administered an anti-immunosuppressive/immunostimulatory agent such as a checkpoint inhibitor. For example, the subject can be further administered an anti-CTLA antibody or anti-PD-1 or anti-PD-L1. Blockade of CTLA-4 or PD-L1 by antibodies can enhance the immune response to cancerous cells in the patient. In particular, CTLA-4 blockade has been shown effective when following a vaccination protocol.

The optimum amount of each antigen to be included in a vaccine composition and the optimum dosing regimen can be determined. For example, an antigen or its variant can be prepared for intravenous (i.v.) injection, sub-cutaneous (s.c.) injection, intradermal (i.d.) injection, intraperitoneal (i.p.) injection, intramuscular (i.m.) injection. Methods of injection include s.c., i.d., i.p., i.m., and i.v. Methods of DNA or RNA injection include i.d., i.m., s.c., i.p. and i.v. Other methods of administration of the vaccine composition are known to those skilled in the art.

A vaccine can be compiled so that the selection, number and/or amount of antigens present in the composition is/are tissue, cancer, infectious disease, and/or patient-specific. For instance, the exact selection of peptides can be guided by expression patterns of the parent proteins in a given tissue or guided by mutation or disease status of a patient. The selection can be dependent on the specific type of cancer, the specific infectious disease, the status of the disease, the goal of the vaccination (e.g., preventative or targeting an ongoing disease), earlier treatment regimens, the immune status of the patient, and, of course, the HLA-haplotype of the patient. Furthermore, a vaccine can contain individualized components, according to personal needs of the particular patient. Examples include varying the selection of antigens according to the expression of the antigen in the particular patient or adjustments for secondary treatments following a first round or scheme of treatment.

A patient can be identified for administration of an antigen vaccine through the use of various diagnostic methods, e.g., patient selection methods described further below. Patient selection can involve identifying mutations in, or expression patterns of, one or more genes. Patient selection can involve identifying the infectious disease of an ongoing infection. Patient selection can involve identifying risk of an infection by an infectious disease. In some cases, patient selection involves identifying the haplotype of the patient. The various patient selection methods can be performed in parallel, e.g., a sequencing diagnostic can identify both the mutations and the haplotype of a patient. The various patient selection methods can be performed sequentially, e.g., one diagnostic test identifies the mutations and separate diagnostic test identifies the haplotype of a patient, and where each test can be the same (e.g., both high-throughput sequencing) or different (e.g., one high-throughput sequencing and the other Sanger sequencing) diagnostic methods. For example, a subject can be haplotyped to determine if they have one or more of the MHC alleles shown in Table 2A, Table 14A, and/or Table 14B for the indicated CTA-associated epitopes and/or one or more of the MHC alleles shown in Table 14C for the indicated KRAS-associated neoepitopes, e.g., to see if they are a candidate for administration of the vaccine systems described herein.

For a composition to be used as a vaccine for cancer or an infectious disease, antigens with similar normal self-peptides that are expressed in high amounts in normal tissues can be avoided or be present in low amounts in a composition described herein. On the other hand, if it is known that the tumor or infected cell of a patient expresses high amounts of a certain antigen, the respective pharmaceutical composition for treatment of this cancer or infection can be present in high amounts and/or more than one antigen specific for this particularly antigen or pathway of this antigen can be included.

Compositions comprising an antigen can be administered to an individual already suffering from cancer. In therapeutic applications, compositions are administered to a subject in an amount sufficient to stimulate an effective CTL response to the tumor antigen and to cure or at least partially arrest symptoms and/or complications. An amount adequate to accomplish this is defined as “therapeutically effective dose.” Amounts effective for this use will depend on, e.g., the composition, the manner of administration, the stage and severity of the disease being treated, the weight and general state of health of the patient, and the judgment of the prescribing physician. It should be kept in mind that compositions can generally be employed in serious disease states, that is, life-threatening or potentially life threatening situations, especially when the cancer has metastasized. In such cases, in view of the minimization of extraneous substances and the relative nontoxic nature of an antigen, it is possible and can be felt desirable by the treating physician to administer substantial excesses of these compositions.

For therapeutic use, administration can begin at the detection or surgical removal of tumors, or begin at the detection or treatment of an infection. This can be followed by boosting doses until at least symptoms are substantially abated and for a period thereafter, or immunity is considered to be provided (e.g., a memory B cell or T cell population, or antigen specific B cells or antibodies are produced).

The pharmaceutical compositions (e.g., vaccine compositions) for therapeutic treatment are intended for parenteral, topical, nasal, oral or local administration. A pharmaceutical compositions can be administered parenterally, e.g., intravenously, subcutaneously, intradermally, or intramuscularly. The compositions can be administered at a site of surgical excision to stimulate a local immune response to a tumor. The compositions can be administered to target specific infected tissues and/or cells of a subject. Disclosed herein are compositions for parenteral administration which comprise a solution of the antigen and vaccine compositions are dissolved or suspended in an acceptable carrier, e.g., an aqueous carrier. A variety of aqueous carriers can be used, e.g., water, buffered water, 0.9% saline, 0.3% glycine, hyaluronic acid and the like. These compositions can be sterilized by conventional, well known sterilization techniques, or can be sterile filtered. The resulting aqueous solutions can be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

Antigens can also be administered via liposomes, which target them to a particular cells tissue, such as lymphoid tissue. Liposomes are also useful in increasing half-life. Liposomes include emulsions, foams, micelles, insoluble monolayers, liquid crystals, phospholipid dispersions, lamellar layers and the like. In these preparations the antigen to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes filled with a desired antigen can be directed to the site of lymphoid cells, where the liposomes then deliver the selected therapeutic/immunogenic compositions. Liposomes can be formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al., Ann. Rev. Biophys. Bioeng. 9; 467 (1980), U.S. Pat. Nos. 4,235,871, 4,501,728, 4,501,728, 4,837,028, and 5,019,369.

For targeting to the immune cells, a ligand to be incorporated into the liposome can include, e.g., antibodies or fragments thereof specific for cell surface determinants of the desired immune system cells. A liposome suspension can be administered intravenously, locally, topically, etc. in a dose which varies according to, inter alia, the manner of administration, the peptide being delivered, and the stage of the disease being treated.

For therapeutic or immunization purposes, nucleic acids encoding a peptide and optionally one or more of the peptides described herein can also be administered to the patient. A number of methods are conveniently used to deliver the nucleic acids to the patient. For instance, the nucleic acid can be delivered directly, as “naked DNA”. This approach is described, for instance, in Wolff et al., Science 247: 1465-1468 (1990) as well as U.S. Pat. Nos. 5,580,859 and 5,589,466. The nucleic acids can also be administered using ballistic delivery as described, for instance, in U.S. Pat. No. 5,204,253. Particles comprised solely of DNA can be administered. Alternatively, DNA can be adhered to particles, such as gold particles. Approaches for delivering nucleic acid sequences can include viral vectors, mRNA vectors, and DNA vectors with or without electroporation.

The nucleic acids can also be delivered complexed to cationic compounds, such as cationic lipids. Lipid-mediated gene delivery methods are described, for instance, in 9618372WOAWO 96/18372; 9324640WOAWO 93/24640; Mannino & Gould-Fogerite, BioTechniques 6(7): 682-691 (1988); U.S. Pat. No. 5,279,833 Rose U.S. Pat. Nos. 5,279,833; 9,106,309WOAWO 91/06309; and Felgner et al., Proc. Natl. Acad. Sci. USA 84: 7413-7414 (1987).

Antigens can also be included in viral vector-based vaccine platforms, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In Vivo Gene Delivery, J. Virol. (1998) 72 (12): 9873-9880). Dependent on the packaging capacity of the above mentioned viral vector-based vaccine platforms, this approach can deliver one or more nucleotide sequences that encode one or more antigen peptides. The sequences may be flanked by non-mutated sequences, may be separated by linkers or may be preceded with one or more sequences targeting a subcellular compartment (See, e.g., Gros et al., Prospective identification of neoantigen-specific lymphocytes in the peripheral blood of melanoma patients, Nat Med. (2016) 22 (4):433-8, Stronen et al., Targeting of cancer neoantigens with donor-derived T cell receptor repertoires, Science. (2016) 352 (6291):1337-41, Lu et al., Efficient identification of mutated cancer antigens recognized by T cells associated with durable tumor regressions, Clin Cancer Res. (2014) 20(13):3401-10). Upon introduction into a host, infected cells express the antigens, and thereby stimulate a host immune (e.g., CTL) response against the peptide(s). Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vaccine vectors useful for therapeutic administration or immunization of antigens, e.g., Salmonella typhi vectors, and the like will be apparent to those skilled in the art from the description herein.

A means of administering nucleic acids uses minigene constructs encoding one or multiple epitopes. To create a DNA sequence encoding the selected CTL epitopes (minigene) for expression in human cells, the amino acid sequences of the epitopes are reverse translated. A human codon usage table is used to guide the codon choice for each amino acid. These epitope-encoding DNA sequences are directly adjoined, creating a continuous polypeptide sequence. To optimize expression and/or immunogenicity, additional elements can be incorporated into the minigene design. Examples of amino acid sequence that could be reverse translated and included in the minigene sequence include: helper T lymphocyte, epitopes, a leader (signal) sequence, and an endoplasmic reticulum retention signal. In addition, MHC presentation of CTL epitopes can be improved by including synthetic (e.g. poly-alanine) or naturally-occurring flanking sequences adjacent to the CTL epitopes. The minigene sequence is converted to DNA by assembling oligonucleotides that encode the plus and minus strands of the minigene. Overlapping oligonucleotides (30-100 bases long) are synthesized, phosphorylated, purified and annealed under appropriate conditions using well known techniques. The ends of the oligonucleotides are joined using T4 DNA ligase. This synthetic minigene, encoding the CTL epitope polypeptide, can then cloned into a desired expression vector.

Purified plasmid DNA can be prepared for injection using a variety of formulations. The simplest of these is reconstitution of lyophilized DNA in sterile phosphate-buffer saline (PBS). A variety of methods have been described, and new techniques can become available. As noted above, nucleic acids are conveniently formulated with cationic lipids. In addition, glycolipids, fusogenic liposomes, peptides and compounds referred to collectively as protective, interactive, non-condensing (PINC) could also be complexed to purified plasmid DNA to influence variables such as stability, intramuscular dispersion, or trafficking to specific organs or cell types.

Also disclosed is a method of manufacturing a vaccine, comprising performing the steps of a method disclosed herein; and producing a vaccine comprising a plurality of antigens or a subset of the plurality of antigens.

Antigens disclosed herein can be manufactured using methods known in the art. For example, a method of producing an antigen or a vector (e.g., a vector including at least one sequence encoding one or more antigens) disclosed herein can include culturing a host cell under conditions suitable for expressing the antigen or vector wherein the host cell comprises at least one polynucleotide encoding the antigen or vector, and purifying the antigen or vector. Standard purification methods include chromatographic techniques, electrophoretic, immunological, precipitation, dialysis, filtration, concentration, and chromatofocusing techniques.

Host cells can include a Chinese Hamster Ovary (CHO) cell, NS0 cell, yeast, or a HEK293 cell. Host cells can be transformed with one or more polynucleotides comprising at least one nucleic acid sequence that encodes an antigen or vector disclosed herein, optionally wherein the isolated polynucleotide further comprises a promoter sequence operably linked to the at least one nucleic acid sequence that encodes the antigen or vector. In certain embodiments the isolated polynucleotide can be cDNA.

### VII. Antigen Use and Administration

A vaccination protocol can be used to dose a subject with one or more antigens. A priming vaccine and a boosting vaccine can be used to dose the subject. Vaccination methods, protocols, and schedules that can be used include, but are not limited to, those described in international application publication WO2021092095, herein incorporated by reference for all purposes.

A priming vaccine, can be based on C68 (e.g., the sequences shown in SEQ ID NO:1 or 2) or SAM (e.g., the sequences shown in SEQ ID NO:3 or 4). A boosting vaccine can also be based on C68 (e.g., the sequences shown in SEQ ID NO:1 or 2) or SAM (e.g., the sequences shown in SEQ ID NO:3 or 4).

Each vector in a prime/boost strategy typically includes a cassette that includes antigens. Cassettes can include about 1-50 antigens, separated by spacers such as the natural sequence that normally surrounds each antigen or other non-natural spacer sequences such as AAY. Cassettes can also include MHCII antigens such a tetanus toxoid antigen and PADRE antigen, which can be considered universal class II antigens. Cassettes can also include a targeting sequence such as a ubiquitin targeting sequence. In addition, each vaccine dose can be administered to the subject in conjunction with (e.g., concurrently, before, or after) an immune modulator. Each vaccine dose can be administered to the subject in conjunction with (e.g., concurrently, before, or after) a checkpoint inhibitor (CPI). CPI's can include those that inhibit CTLA4, PD1, and/or PDL1 such as antibodies or antigen-binding portions thereof. Such antibodies can include tremelimumab or durvalumab. Each vaccine dose can be administered to the subject in conjunction with (e.g., concurrently, before, or after) a cytokine, such as IL-2, IL-7, IL-12 (including IL-12 p35, p40, p70, and/or p70-fusion constructs), IL-15, or IL-21. Each vaccine dose can be administered to the subject in conjunction with (e.g., concurrently, before, or after) a modified cytokine (e.g., pegIL-2).

A priming vaccine can be injected (e.g., intramuscularly) in a subject. Bilateral injections per dose can be used. For example, one or more injections of ChAdV68 (C68) can be used (e.g., total dose 1×10¹² viral particles); one or more injections of SAM vectors at low vaccine dose selected from the range 0.001 to 1 ug RNA, in particular 0.1 or 1 ug can be used; or one or more injections of SAM vectors at high vaccine dose selected from the range 1 to 100 ug RNA, in particular 10 or 100 ug can be used.

A vaccine boost (boosting vaccine) can be injected (e.g., intramuscularly) after prime vaccination. A boosting vaccine can be administered about every 1, 2, 3, 4, 5, 6, 7, 8, 9, or 10 weeks, e.g., every 4 weeks and/or 8 weeks after the prime. Bilateral injections per dose can be used. For example, one or more injections of ChAdV68 (C68) can be used (e.g., total dose 1×10¹² viral particles); one or more injections of SAM vectors at low vaccine dose selected from the range 0.001 to 1 ug RNA, in particular 0.1 or 1 ug can be used; or one or more injections of SAM vectors at high vaccine dose selected from the range 1 to 100 ug RNA, in particular 10 or 100 ug can be used.

Administration of vaccine systems described herein can include co-administration of separate vectors encoding distinct (neo)epitopes. For example, administration can include co-administration of a vector that includes a CTA-encoding nucleic acid sequence and a separate vector that includes a KRAS-encoding nucleic acid sequence. Co-administration can include administering a mixture of the separate vectors (a “blended” vaccine), such as vectors formulated in the same pharmaceutical composition. Illustrative examples of mixtures include vectors formulated in the same nanoparticle delivery vehicle, e.g., a lipid nanoparticle (LNP), or vectors formulated in separate nanoparticle delivery vehicles and then subsequently mixed, e.g., pre-mixed by a manufacturer or mixed immediately prior to administration. Other illustrative examples of mixtures include mixtures of separate adenoviral delivery vectors. Co-administration can include multiple injections of a single vector or a mixture of separate vectors, such as bilateral administration of either a single vector or a mixture of separate vectors. Co-administration can include administering the separate vectors separately, e.g., each vector administered as a separate injection, such as separate bilateral injections of each vector (e.g., an injection of a first vector on one side of a body and an injection of a second, distinct vector on the other side of the body).

Anti-CTLA-4 (e.g., tremelimumab) can also be administered to the subject. For example, anti-CTLA4 can be administered subcutaneously near the site of the intramuscular vaccine injection (ChAdV68 prime or SAM low doses) to ensure drainage into the same lymph node. Tremelimumab is a selective human IgG2 mAb inhibitor of CTLA-4. Target Anti-CTLA-4 (tremelimumab) subcutaneous dose is typically 70-75 mg (in particular 75 mg) with a dose range of, e.g., 1-100 mg or 5-420 mg.

In certain instances an anti-PD-L1 antibody can be used such as durvalumab (MEDI 4736). Durvalumab is a selective, high affinity human IgG1 mAb that blocks PD-L1 binding to PD-1 and CD80. Durvalumab is generally administered at 20 mg/kg i.v. every 4 weeks.

Immune monitoring can be performed before, during, and/or after vaccine administration. Such monitoring can inform safety and efficacy, among other parameters.

To perform immune monitoring, PBMCs are commonly used. PBMCs can be isolated before prime vaccination, and after prime vaccination (e.g. 4 weeks and 8 weeks). PBMCs can be harvested just prior to boost vaccinations and after each boost vaccination (e.g. 4 weeks and 8 weeks).

Immune responses, such as T cell responses and B cell responses, can be assessed as part of an immune monitoring protocol. For example, the ability of a vaccine composition described herein to stimulate an immune response can be monitored and/or assessed. As used herein, “stimulate an immune response” refers to any increase in a immune response, such as initiating an immune response (e.g., a priming vaccine stimulating the initiation of an immune response in a naïve subject) or enhancement of an immune response (e.g., a boosting vaccine stimulating the enhancement of an immune response in a subject having a pre-existing immune response to an antigen, such as a pre-existing immune response initiated by a priming vaccine). T cell responses can be measured using one or more methods known in the art such as ELISpot, intracellular cytokine staining, cytokine secretion and cell surface capture, T cell proliferation, MHC multimer staining, or by cytotoxicity assay. T cell responses to epitopes encoded in vaccines can be monitored from PBMCs by measuring induction of cytokines, such as IFN-gamma, using an ELISpot assay. Specific CD4 or CD8 T cell responses to epitopes encoded in vaccines can be monitored from PBMCs by measuring induction of cytokines captured intracellularly or extracellularly, such as IFN-gamma, using flow cytometry. Specific CD4 or CD8 T cell responses to epitopes encoded in the vaccines can be monitored from PBMCs by measuring T cell populations expressing T cell receptors specific for epitope/MHC class I complexes using MHC multimer staining. Specific CD4 or CD8 T cell responses to epitopes encoded in the vaccines can be monitored from PBMCs by measuring the ex vivo expansion of T cell populations following 3H-thymidine, bromodeoxyuridine and carboxyfluoresceine-diacetate-succinimidylester (CFSE) incorporation. The antigen recognition capacity and lytic activity of PBMC-derived T cells that are specific for epitopes encoded in vaccines can be assessed functionally by chromium release assay or alternative colorimetric cytotoxicity assays.

B cell responses can be measured using one or more methods known in the art such as assays used to determine B cell differentiation (e.g., differentiation into plasma cells), B cell or plasma cell proliferation, B cell or plasma cell activation (e.g., upregulation of costimulatory markers such as CD80 or CD86), antibody class switching, and/or antibody production (e.g., an ELISA). Antibodies can also be assessed for function, such as assessed for neutralizing ability.

Disease status of a subject can be monitored following administration of any of the vaccine compositions described herein. For example, disease status may be monitored using isolated cell-free DNA (cfDNA) from a subject. In addition, the efficacy of a vaccine therapy may be monitored using isolated cfDNA from a subject. cfDNA monitoring can include the steps of: a. isolating or having isolated cfDNA from a subject; b. sequencing or having sequenced the isolated cfDNA; c. determining or having determined a frequency of one or more mutations in the cfDNA relative to a wild-type germline nucleic acid sequence of the subject, and d. assessing or having assessed from step (c) the status of a disease in the subject. The method can also include, following step (c) above, d. performing more than one iteration of steps (a)-(c) for the given subject and comparing the frequency of the one or more mutations determined in the more than one iterations; and f. assessing or having assessed from step (d) the status of a disease in the subject. The more than one iterations can be performed at different time points, such as a first iteration of steps (a)-(c) performed prior to administration of the vaccine composition and a second iteration of steps (a)-(c) is performed subsequent to administration of the vaccine composition. Step (c) can include comparing: the frequency of the one or more mutations determined in the more than one iterations, or the frequency of the one or more mutations determined in the first iteration to the frequency of the one or more mutations determined in the second iteration. An increase in the frequency of the one or more mutations determined in subsequent iterations or the second iteration can be assessed as disease progression. A decrease in the frequency of the one or more mutations determined in subsequent iterations or the second iteration can be assessed as a response. In some aspects, the response is a Complete Response (CR) or a Partial Response (PR). A therapy can be administered to a subject following an assessment step, such as where assessment of the frequency of the one or more mutations in the cfDNA indicates the subject has the disease. The cfDNA isolation step can use centrifugation to separate cfDNA from cells or cellular debris. cfDNA can be isolated from whole blood, such as by separating the plasma layer, buffy coat, and red bloods. cfDNA sequencing can use next generation sequencing (NGS), Sanger sequencing, duplex sequencing, whole-exome sequencing, whole-genome sequencing, de novo sequencing, phased sequencing, targeted amplicon sequencing, shotgun sequencing, or combinations thereof, and may include enriching the cfDNA for one or more polynucleotide regions of interest prior to sequencing (e.g., polynucleotides known or suspected to encode the one or more mutations, coding regions, and/or tumor exome polynucleotides). Enriching the cfDNA may include hybridizing one or more polynucleotide probes, which may be modified (e.g., biotinylated), to the one or more polynucleotide regions of interest. In general, any number of mutations may be monitored simultaneously or in parallel.

### VIII. Isolation and Detection of HLA Peptides

Isolation of HLA-peptide molecules was performed using classic immunoprecipitation (IP) methods after lysis and solubilization of the tissue sample (55-58). A clarified lysate was used for HLA specific IP.

Immunoprecipitation was performed using antibodies coupled to beads where the antibody is specific for HLA molecules. For a pan-Class I HLA immunoprecipitation, a pan-Class I CR antibody is used, for Class II HLA-DR, an HLA-DR antibody is used. Antibody is covalently attached to NHS-sepharose beads during overnight incubation. After covalent attachment, the beads were washed and aliquoted for IP. (59, 60) Immunoprecipitations can also be performed with antibodies that are not covalently attached to beads. Typically this is done using sepharose or magnetic beads coated with Protein A and/or Protein G to hold the antibody to the column. Some antibodies that can be used to selectively enrich MHC/peptide complex are listed below.

| Antibody Name | Specificity |
|---|---|
| W6/32 | Class I HLA-A, B, C |
| L243 | Class II - HLA-DR |
| Tu36 | Class II - HLA-DR |
| LN3 | Class II - HLA-DR |
| Tu39 | Class II - HLA-DR, DP, DQ |

The clarified tissue lysate is added to the antibody beads for the immunoprecipitation. After immunoprecipitation, the beads are removed from the lysate and the lysate stored for additional experiments, including additional IPs. The IP beads are washed to remove non-specific binding and the HLA/peptide complex is eluted from the beads using standard techniques. The protein components are removed from the peptides using a molecular weight spin column or C18 fractionation. The resultant peptides are taken to dryness by SpeedVac evaporation and in some instances are stored at −20 C prior to MS analysis.

Dried peptides are reconstituted in an HPLC buffer suitable for reverse phase chromatography and loaded onto a C-18 microcapillary HPLC column for gradient elution in a Fusion Lumos mass spectrometer (Thermo). MS1 spectra of peptide mass/charge (m/z) were collected in the Orbitrap detector at high resolution followed by MS2 low resolution scans collected in the ion trap detector after HCD fragmentation of the selected ion. Additionally, MS2 spectra can be obtained using either CID or ETD fragmentation methods or any combination of the three techniques to attain greater amino acid coverage of the peptide. MS2 spectra can also be measured with high resolution mass accuracy in the Orbitrap detector with targeted method known as parallel reaction monitoring. In targeted PRM, specific peptide precursor ions are isolated in the Orbitrap detector and all resulting HCD fragmentation ions are scanned across the elution of the peptide peak. This enables both peptide identification and quantitation of endogenous peptide in the presence of a co-injected stabile isotopically labeled peptide standard.

MS2 spectra from each analysis are searched against a protein database using Comet (61, 62) and the peptide identification are scored using Percolator (63-65). Additional sequencing is performed using PEAKS studio (Bioinformatics Solutions Inc.) and other search engines or sequencing methods can be used including spectral matching and de novo sequencing (97). Targeted MS1 and MS2 spectra are processed through Skyline (104).

### VIII.B.1. MS Limit of Detection Studies in Support of Comprehensive HLA Peptide Sequencing

Using the peptide YVYVADVAAK it was determined what the limits of detection are using different amounts of peptide loaded onto the LC column. The amounts of peptide tested were 1 μmol, 100 fmol, 10 fmol, 1 fmol, and 100 amol. (Table 1 and data not shown). These results indicate that the lowest limit of detection (LoD) is in the attomol range (10⁻¹⁸), that the dynamic range spans five orders of magnitude, and that the signal to noise appears sufficient for sequencing at low femtomol ranges (10⁻¹⁵).

TABLE 1

| | | |
|---|---|---|
| | | Copies/Cell |
| Peptide m/z | Loaded on Column | in 1e9cells |

| | | | |
|---|---|---|---|
| 566.830 | 1 | pmol | 600 |
| 562.823 | 100 | fmol | 60 |
| 559.816 | 10 | fmol | 6 |
| 556.810 | 1 | fmol | 0.6 |
| 553.802 | 100 | amol | 0.06 |

### IX. Presentation Model

Presentation models can be used to identify likelihoods of peptide presentation in patients. Various presentation models are known to those skilled in the art, for example the presentation models described in more detail in U.S. Pat. No. 10,055,540, US Application Pub. No. US20200010849A1 and US20110293637, and international patent application publications WO/2018/195357, WO/2018/208856, and WO2016187508, each herein incorporated by reference, in their entirety, for all purposes.

### X. Training Module

Training modules can be used to construct one or more presentation models based on training data sets that generate likelihoods of whether peptide sequences will be presented by MHC alleles associated with the peptide sequences. Various training modules are known to those skilled in the art, for example the presentation models described in more detail in U.S. Pat. No. 10,055,540, US Application Pub. No. US20200010849A1, and international patent application publications WO/2018/195357 and WO/2018/208856, each herein incorporated by reference, in their entirety, for all purposes. A training module can construct a presentation model to predict presentation likelihoods of peptides on a per-allele basis. A training module can also construct a presentation model to predict presentation likelihoods of peptides in a multiple-allele setting where two or more MHC alleles are present.

### XI. Prediction Module

A prediction module can be used to receive sequence data and select candidate antigens in the sequence data using a presentation model. Specifically, the sequence data may be DNA sequences, RNA sequences, and/or protein sequences extracted from tumor tissue cells of patients, infected cells patients, or infectious disease organisms themselves. A prediction module may identify candidate neoantigens that are mutated peptide sequences by comparing sequence data extracted from normal tissue cells of a patient with the sequence data extracted from tumor tissue cells of the patient to identify portions containing one or more mutations. A prediction module may identify candidate antigens that are pathogen-derived peptides, virally-derived peptides, bacterially-derived peptides, fungally-derived peptides, and parasitically-derived peptides, such as by comparing sequence data extracted from normal tissue cells of a patient with the sequence data extracted from infected cells of the patient to identify portions containing one or more infectious disease organism associated antigens. A prediction module may identify candidate antigens that have altered expression in a tumor cell or cancerous tissue in comparison to a normal cell or tissue by comparing sequence data extracted from normal tissue cells of a patient with the sequence data extracted from tumor tissue cells of the patient to identify improperly expressed candidate antigens. A prediction module may identify candidate antigens that are expressed in an infected cell or infected tissue in comparison to a normal cell or tissue by comparing sequence data extracted from normal tissue cells of a patient with the sequence data extracted from infected tissue cells of the patient to identify expressed candidate antigens (e.g., identifying expressed polynucleotides and/or polypeptides specific to an infectious disease).

A presentation module can apply one or more presentation model to processed peptide sequences to estimate presentation likelihoods of the peptide sequences. Specifically, the prediction module may select one or more candidate antigen peptide sequences that are likely to be presented on tumor HLA molecules or infected cell HLA molecules by applying presentation models to the candidate antigens. In one implementation, the presentation module selects candidate antigen sequences that have estimated presentation likelihoods above a predetermined threshold. In another implementation, the presentation model selects the N candidate antigen sequences that have the highest estimated presentation likelihoods (where N is generally the maximum number of epitopes that can be delivered in a vaccine). A vaccine including the selected candidate antigens for a given subject can be injected into the subject to stimulate immune responses.

### XI.B. Cassette Design Module

### XI.B.1 Overview

A cassette design module can be used to generate a vaccine cassette sequence based on selected candidate peptides for injection into a patient. Various cassette design modules are known to those skilled in the art, for example the cassette design modules described in more detail in U.S. Pat. No. 10,055,540, US Application Pub. No. US20200010849A1, and international patent application publications WO/2018/195357 and WO/2018/208856, each herein incorporated by reference, in their entirety, for all purposes.

A set of therapeutic epitopes may be generated based on the selected peptides determined by a prediction module associated with presentation likelihoods above a predetermined threshold, where the presentation likelihoods are determined by the presentation models. However it is appreciated that in other embodiments, the set of therapeutic epitopes may be generated based on any one or more of a number of methods (alone or in combination), for example, based on binding affinity or predicted binding affinity to HLA class I or class II alleles of the patient, binding stability or predicted binding stability to HLA class I or class II alleles of the patient, random sampling, and the like.

Therapeutic epitopes may correspond to selected peptides themselves. Therapeutic epitopes may also include C- and/or N-terminal flanking sequences in addition to the selected peptides. N- and C-terminal flanking sequences can be the native N- and C-terminal flanking sequences of the therapeutic vaccine epitope in the context of its source protein. Therapeutic epitopes can represent a fixed-length epitope Therapeutic epitopes can represent a variable-length epitope, in which the length of the epitope can be varied depending on, for example, the length of the C- or N-flanking sequence. For example, the C-terminal flanking sequence and the N-terminal flanking sequence can each have varying lengths of 2-5 residues, resulting in 16 possible choices for the epitope.

A cassette design module can also generate cassette sequences by taking into account presentation of junction epitopes that span the junction between a pair of therapeutic epitopes in the cassette. Junction epitopes are novel non-self but irrelevant epitope sequences that arise in the cassette due to the process of concatenating therapeutic epitopes and linker sequences in the cassette. The novel sequences of junction epitopes are different from the therapeutic epitopes of the cassette themselves.

A cassette design module can generate a cassette sequence that reduces the likelihood that junction epitopes are presented in the patient. Specifically, when the cassette is injected into the patient, junction epitopes have the potential to be presented by HLA class I or HLA class II alleles of the patient, and stimulate a CD8 or CD4 T-cell response, respectively. Such reactions are often times undesirable because T-cells reactive to the junction epitopes have no therapeutic benefit, and may diminish the immune response to the selected therapeutic epitopes in the cassette by antigenic competition.⁷⁶

A cassette design module can iterate through one or more candidate cassettes, and determine a cassette sequence for which a presentation score of junction epitopes associated with that cassette sequence is below a numerical threshold. The junction epitope presentation score is a quantity associated with presentation likelihoods of the junction epitopes in the cassette, and a higher value of the junction epitope presentation score indicates a higher likelihood that junction epitopes of the cassette will be presented by HLA class I or HLA class II or both.

In one embodiment, a cassette design module may determine a cassette sequence associated with the lowest junction epitope presentation score among the candidate cassette sequences.

A cassette design module may iterate through one or more candidate cassette sequences, determine the junction epitope presentation score for the candidate cassettes, and identify an optimal cassette sequence associated with a junction epitope presentation score below the threshold.

A cassette design module may further check the one or more candidate cassette sequences to identify if any of the junction epitopes in the candidate cassette sequences are self-epitopes for a given patient for whom the vaccine is being designed. To accomplish this, the cassette design module checks the junction epitopes against a known database such as BLAST. In one embodiment, the cassette design module may be configured to design cassettes that avoid junction self-epitopes.

A cassette design module can perform a brute force approach and iterate through all or most possible candidate cassette sequences to select the sequence with the smallest junction epitope presentation score. However, the number of such candidate cassettes can be prohibitively large as the capacity of the vaccine increases. For example, for a vaccine capacity of 20 epitopes, the cassette design module has to iterate through ˜10¹⁸ possible candidate cassettes to determine the cassette with the lowest junction epitope presentation score. This determination may be computationally burdensome (in terms of computational processing resources required), and sometimes intractable, for the cassette design module to complete within a reasonable amount of time to generate the vaccine for the patient. Moreover, accounting for the possible junction epitopes for each candidate cassette can be even more burdensome. Thus, a cassette design module may select a cassette sequence based on ways of iterating through a number of candidate cassette sequences that are significantly smaller than the number of candidate cassette sequences for the brute force approach.

A cassette design module can generate a subset of randomly or at least pseudo-randomly generated candidate cassettes, and selects the candidate cassette associated with a junction epitope presentation score below a predetermined threshold as the cassette sequence. Additionally, the cassette design module may select the candidate cassette from the subset with the lowest junction epitope presentation score as the cassette sequence. For example, the cassette design module may generate a subset of ˜1 million candidate cassettes for a set of 20 selected epitopes, and select the candidate cassette with the smallest junction epitope presentation score. Although generating a subset of random cassette sequences and selecting a cassette sequence with a low junction epitope presentation score out of the subset may be sub-optimal relative to the brute force approach, it requires significantly less computational resources thereby making its implementation technically feasible. Further, performing the brute force method as opposed to this more efficient technique may only result in a minor or even negligible improvement in junction epitope presentation score, thus making it not worthwhile from a resource allocation perspective. A cassette design module can determine an improved cassette configuration by formulating the epitope sequence for the cassette as an asymmetric traveling salesman problem (TSP). Given a list of nodes and distances between each pair of nodes, the TSP determines a sequence of nodes associated with the shortest total distance to visit each node exactly once and return to the original node. For example, given cities A, B, and C with known distances between each other, the solution of the TSP generates a closed sequence of cities, for which the total distance traveled to visit each city exactly once is the smallest among possible routes. The asymmetric version of the TSP determines the optimal sequence of nodes when the distance between a pair of nodes are asymmetric. For example, the “distance” for traveling from node A to node B may be different from the “distance” for traveling from node B to node A. By solving for an improved optimal cassette using an asymmetric TSP, the cassette design module can find a cassette sequence that results in a reduced presentation score across the junctions between epitopes of the cassette. The solution of the asymmetric TSP indicates a sequence of therapeutic epitopes that correspond to the order in which the epitopes should be concatenated in a cassette to minimize the junction epitope presentation score across the junctions of the cassette. A cassette sequence determined through this approach can result in a sequence with significantly less presentation of junction epitopes while potentially requiring significantly less computational resources than the random sampling approach, especially when the number of generated candidate cassette sequences is large. Illustrative examples of different computational approaches and comparisons for optimizing cassette design are described in more detail in U.S. Pat. No. 10,055,540, US Application Pub. No. US20200010849A1, and international patent application publications WO/2018/195357 and WO/2018/208856, each herein incorporated by reference, in their entirety, for all purposes.

Shared (neo)antigen sequences for inclusion in a shared antigen vaccine and appropriate patients for treatment with such vaccine can be chosen by one of skill in the art, e.g., as described in U.S. application Ser. No. 17/058,128, herein incorporated by reference for all purposes. Mass spectrometry (MS) validation of candidate shared (neo)antigens can performed as part of the selection process.

### XIII. Example Computer

A computer can be used for any of the computational methods described herein. One skilled in the art will recognize a computer can have different architectures. Examples of computers are known to those skilled in the art, for example the computers described in more detail in U.S. Pat. No. 10,055,540, US Application Pub. No. US20200010849A1, and international patent application publications WO/2018/195357 and WO/2018/208856, each herein incorporated by reference, in their entirety, for all purposes.

### ADDITIONAL EMBODIMENTS

Below are examples of specific embodiments:

Embodiment 1. An antigen-encoding composition, wherein the antigen-encoding composition comprises (a) a Cancer Testis Antigen (CTA)-encoding nucleic acid sequence encoding a CTA-associated MHC class I epitope and (b) a KRAS-encoding nucleic acid sequence encoding a KRAS-associated MHC class I epitope:
wherein each of the CTA-encoding nucleic acid sequence and the KRAS-encoding nucleic acid sequence is described, from 5′ to 3′, by the formula:

(Ex-(ENn)y)z
wherein E represents a nucleotide sequence comprising a distinct epitope-encoding nucleic acid sequence,
n represents the number of separate distinct epitope-encoding nucleic acid sequences and is any integer including 0,
EN represents a nucleotide sequence comprising the separate distinct epitope-encoding nucleic acid sequence for each corresponding n,
for each iteration of z: x=0 or 1, y=0 or 1 for each n, and at least one of x or y=1, and
z=1 or greater, optionally wherein the CTA-encoding nucleic acid sequence and/or the KRAS-encoding nucleic acid sequence comprises at least two iterations of E, a given EN, or a combination thereof.

Embodiment 2. The composition of embodiment 1, wherein the CTA-encoding nucleic acid sequence and the KRAS-encoding nucleic acid sequence are encoded in the same cassette.

Embodiment 3. The composition of embodiment 2, wherein the CTA-encoding nucleic acid sequence and the KRAS-encoding nucleic acid sequence are linked together by a 2A ribosome skipping sequence.

Embodiment 4. The composition of embodiment 2, wherein the CTA-encoding nucleic acid sequence and the KRAS-encoding nucleic acid sequence are each independently operably linked to a separate promoter.

Embodiment 5. The composition of embodiment 1, wherein the CTA-encoding nucleic acid sequence and the KRAS-encoding nucleic acid sequence are encoded on separate vectors.

Embodiment 6. An antigen-encoding cassette, or a polypeptide sequence encoded by the cassette, wherein the antigen-encoding cassette comprises at least one antigen-encoding nucleic acid sequence described, from 5′ to 3′, by the formula:

(Ex-(ENn)y)z
wherein E represents a nucleotide sequence comprising a distinct epitope-encoding nucleic acid sequence,
n represents the number of separate distinct epitope-encoding nucleic acid sequences and is any integer including 0,
EN represents a nucleotide sequence comprising the separate distinct epitope-encoding nucleic acid sequence for each corresponding n,
for each iteration of z: x=0 or 1, y=0 or 1 for each n, and at least one of x or y=1, and
z=1 or greater, optionally wherein the antigen-encoding nucleic acid sequence comprises at least two iterations of E, a given EN, or a combination thereof, and
at least one of the distinct epitope-encoding nucleic acid sequences encodes a distinct shared Cancer Testis Antigen (CTA)-associated MHC class I epitope, optionally wherein at least one of the distinct epitope-encoding nucleic acid sequences encoding the CTA comprises at least two iterations.

Embodiment 7. The composition of any one of embodiments 1-6, wherein the CTA-associated MHC class I epitope is selected from the group consisting of: a MAGEA1 MHC class I epitope encoding nucleic acid sequence, a MAGEA3 MHC class I epitope encoding nucleic acid sequence, a MAGEA4 MHC class I epitope encoding nucleic acid sequence, a MAGEA6 MHC class I epitope encoding nucleic acid sequence, a MAGEA8 MHC class I epitope encoding nucleic acid sequence, a MAGEA11 MHC class I epitope encoding nucleic acid sequence, a MAGEB2 MHC class I epitope encoding nucleic acid sequence, a CTCFL MHC class I epitope encoding nucleic acid sequence, and a CT83 MHC class I epitope encoding nucleic acid sequence.

Embodiment 8. The composition of embodiment 7, wherein the CTA-associated MHC class I epitope is selected from the group consisting of: FVQENYLEY, EVDPTSHSY, NTDNNLAVY, EVDPIGHLY, GVYDGREHTV, ALREEGEGV, KVLEYVIKV, GVYDGEEHSV, KLVELEHTL, AETSYVKVL, KVLEHVVRV, EADPTGHSY, SALPTTISF, GVYDGREHTVY, TQHFVQENY, EYVIKVSAR, LVRPSSSGL, GEMSSNSTAL, TVYGEPRKL, ALAETSYVK, TSYVKVLEH, YPSLREAAL, ALLEEEEGV, GPRQSLQQC, IAYPSLREAAL, and MEVDPIGHL.

Embodiment 9. The composition of any one of embodiments 1-8, wherein each of the distinct epitope-encoding nucleic acid sequences comprises at least two iterations of a distinct shared Cancer Testis Antigen (CTA)-associated MHC class I epitope.

Embodiment 10. The composition of any one of embodiments 1-9, wherein the at least one distinct epitope-encoding nucleic acid sequences encoding the CTA-associated MHC class I epitope and/or the KRAS-associated MHC class I epitope comprises at least 3, at least 4, at least 5, at least 6, at least 7 iterations, or at least 8 iterations.

Embodiment 11. The composition of any one of embodiments 1-9, wherein the at least one distinct epitope-encoding nucleic acid sequences encoding the CTA-associated MHC class I epitope and/or the KRAS-associated MHC class I epitope comprises at least 3 iterations.

Embodiment 12. The composition of any one of embodiments 1-9, wherein the at least one distinct epitope-encoding nucleic acid sequences encoding the CTA-associated MHC class I epitope and/or the KRAS-associated MHC class I epitope comprises at least 4 iterations.

Embodiment 13. The composition of any one of the above embodiments, wherein the CTA-encoding nucleic acid sequence encodes:
each of a MAGEA1 MHC class I epitope encoding nucleic acid sequence, a MAGEA4 MHC class I epitope encoding nucleic acid sequence, a MAGEB2 MHC class I epitope encoding nucleic acid sequence, a MAGEA11 MHC class I epitope encoding nucleic acid sequence, a MAGEA3 MHC class I epitope encoding nucleic acid sequence, and a CT83 MHC class I epitope encoding nucleic acid sequence,
each of a MAGEA6 MHC class I epitope encoding nucleic acid sequence, a MAGEA11 MHC class I epitope encoding nucleic acid sequence, a MAGEA3 MHC class I epitope encoding nucleic acid sequence, and a CT83 MHC class I epitope encoding nucleic acid sequence,
each of a MAGEA1 MHC class I epitope encoding nucleic acid sequence, a MAGEA4 MHC class I epitope encoding nucleic acid sequence, a MAGEB2 MHC class I epitope encoding nucleic acid sequence, a MAGEA11 MHC class I epitope encoding nucleic acid sequence, and a CT83 MHC class I epitope encoding nucleic acid sequence, or
each of a MAGEA3 MHC class I epitope encoding nucleic acid sequence, a MAGEA4 MHC class I epitope encoding nucleic acid sequence, a MAGEB2 MHC class I epitope encoding nucleic acid sequence, a MAGEA11 MHC class I epitope encoding nucleic acid sequence, and a CT83 MHC class I epitope encoding nucleic acid sequence.

Embodiment 14. The composition of 13, wherein each of the CTA-associated MHC class I epitope encoding nucleic acid sequences comprises at least two iterations.

Embodiment 15. The composition of any one of embodiments 1-14, wherein each E or EN independently comprises a nucleotide sequence described, from 5′ to 3′, by the formula (L5b-Nc-L3d),
wherein N comprises the distinct epitope-encoding nucleic acid sequence associated with each E or EN, where c=1,
L5 comprises a 5′ linker sequence, where b=0 or 1, and
L3 comprises a 3′ linker sequence, where d=0 or 1.

Embodiment 16. The composition of embodiment 15, wherein
each N encodes an epitope 7-15 amino acids in length,
L5 is a native 5′ linker sequence that encodes a native N-terminal amino acid sequence of the epitope, and wherein the 5′ linker sequence encodes a peptide that is at least 2 amino acids in length, and
L3 is a native 3′ linker sequence that encodes a native C-terminal amino acid sequence of the epitope, and wherein the 3′ linker sequence encodes a peptide that is at least 2 amino acids in length.

Embodiment 17. The composition of any one of embodiments 1-16, wherein each E and EN encodes an epitope at least 7 amino acids in length.

Embodiment 18. The composition of any one of embodiments 1-16, wherein each E and EN encodes an epitope 7-15 amino acids in length.

Embodiment 19. The composition of any one of embodiments 1-18, wherein each E and EN is a nucleotide sequence at least 21 nucleotides in length.

Embodiment 20. The composition of any one of embodiments 1-18, wherein each E and EN is a nucleotide sequence 75 nucleotides in length.

Embodiment 21. The composition of any one of the above embodiments, wherein at least one of the distinct epitope-encoding nucleic acid sequences encodes a distinct KRAS-associated MHC class I neoepitope.

Embodiment 22. The composition of embodiment 21, wherein one or more of the distinct KRAS-associated MHC class I neoepitopes independently comprises a KRAS G12C mutation, a KRAS G12V mutation, a KRAS G12D mutation, or a KRAS Q61H mutation.

Embodiment 23. The composition of embodiment 21, wherein one or more of the distinct KRAS-associated MHC class I neoepitopes independently comprises a KRAS G12C mutation or a KRAS G12V mutation.

Embodiment 24. The composition of embodiment 21, wherein one or more of the distinct KRAS-associated MHC class I neoepitopes independently comprises each of a KRAS G12C mutation and a KRAS G12V mutation.

Embodiment 25. The composition of any one of embodiments 21-24, wherein the distinct KRAS-associated MHC class I neoepitope comprising the KRAS G12C mutation is selected from the group consisting of KLVVVGACGV, VVVGACGVGK, GACGVGKSAL, and combinations thereof.

Embodiment 26. The composition of any one of embodiments 21-25, wherein the distinct KRAS-associated MHC class I neoepitope comprising the KRAS G12V mutation is selected from the group consisting of KLVVVGAVGV, VVVGAVGVGK, AVGVGKSAL, GAVGVGKSAL, and combinations thereof.

Embodiment 27. The composition of any one of embodiments 21-26, wherein at least one of the distinct epitope-encoding nucleic acid sequences encoding the distinct KRAS-associated MHC class I neoepitope comprises at least two iterations of the distinct epitope-encoding nucleic acid sequences encoding the distinct KRAS-associated MHC class I neoepitope.

Embodiment 28. The composition of any one of embodiments 21-26, wherein each of the distinct epitope-encoding nucleic acid sequences encoding the distinct KRAS-associated MHC class I neoepitope comprises at least two iterations of the distinct epitope-encoding nucleic acid sequences encoding the distinct KRAS-associated MHC class I neoepitope.

Embodiment 29. A composition for delivery of an antigen expression system, comprising:
the antigen expression system,
wherein the antigen expression system comprises one or more vectors,
the one or more vectors comprising (A) a Cancer Testis Antigen (CTA)-encoding nucleic acid sequence encoding a CTA-associated MHC class I epitope and (B) a KRAS-encoding nucleic acid sequence encoding a KRAS-associated MHC class I epitope,
wherein each of the one or more vectors independently comprise:
(a) a vector backbone, wherein the backbone comprises:
(i) at least one promoter nucleotide sequence, and
(ii) optionally, at least one polyadenylation (poly(A)) sequence; and
(b) a cassette, wherein the cassette comprises:
(i) at least one antigen-encoding nucleic acid sequence, comprising:
(I) at least one epitope-encoding nucleic acid sequence the CTA-associated MHC class I epitope and/or the KRAS-associated MHC class I epitope, optionally wherein at least one of the epitope-encoding nucleic acid sequences encoding the CTA-associated MHC class I epitope and/or the KRAS-associated MHC class I epitope comprises at least two iterations, and
wherein each of the epitope-encoding nucleic acid sequences comprises;
(A) optionally, a 5′ linker sequence, and
(B) optionally, a 3′ linker sequence;
(ii) optionally, a second promoter nucleotide sequence operably linked to the antigen-encoding nucleic acid sequence; and
(iii) optionally, at least one MHC class II epitope-encoding nucleic acid sequence;
(iv) optionally, at least one nucleic acid sequence encoding a GPGPG amino acid linker sequence (SEQ ID NO:56); and
(v) optionally, at least one second poly(A) sequence, wherein the second poly(A) sequence is a native poly(A) sequence or an exogenous poly(A) sequence to the vector backbone,
wherein if the second promoter nucleotide sequence is absent, the antigen-encoding nucleic acid sequence is operably linked to the at least one promoter nucleotide sequence.

Embodiment 30. A composition for delivery of an antigen expression system, comprising:
the antigen expression system,
wherein the antigen expression system comprises one or more vectors,
the one or more vectors comprising:
(a) a vector backbone, wherein the backbone comprises:
(i) at least one promoter nucleotide sequence, and
(ii) optionally, at least one polyadenylation (poly(A)) sequence; and
(b) a cassette, wherein the cassette comprises:
(i) at least one antigen-encoding nucleic acid sequence, comprising:
(I) at least one epitope-encoding nucleic acid sequence encodes a CTA-associated MHC class I epitope, optionally wherein at least one of the epitope-encoding nucleic acid sequences encoding the CTA comprises at least two iterations, and wherein each of the epitope-encoding nucleic acid sequences comprises;
(A) optionally, a 5′ linker sequence, and
(B) optionally, a 3′ linker sequence;
(ii) optionally, a second promoter nucleotide sequence operably linked to the antigen-encoding nucleic acid sequence; and
(iii) optionally, at least one MHC class II epitope-encoding nucleic acid sequence;
(iv) optionally, at least one nucleic acid sequence encoding a GPGPG amino acid linker sequence (SEQ ID NO:56); and
(v) optionally, at least one second poly(A) sequence, wherein the second poly(A) sequence is a native poly(A) sequence or an exogenous poly(A) sequence to the vector backbone,
wherein if the second promoter nucleotide sequence is absent, the antigen-encoding nucleic acid sequence is operably linked to the at least one promoter nucleotide sequence.

Embodiment 31. The composition of embodiment 29 or 30, wherein each of the CTA-associated MHC class I epitope is selected from the group consisting of: a MAGEA1 MHC class I epitope encoding nucleic acid sequence, a MAGEA3 MHC class I epitope encoding nucleic acid sequence, a MAGEA4 MHC class I epitope encoding nucleic acid sequence, a MAGEA6 MHC class I epitope encoding nucleic acid sequence, a MAGEA8 MHC class I epitope encoding nucleic acid sequence, a MAGEA11 MHC class I epitope encoding nucleic acid sequence, a MAGEB2 MHC class I epitope encoding nucleic acid sequence, a CTCFL MHC class I epitope encoding nucleic acid sequence, and a CT83 MHC class I epitope encoding nucleic acid sequence, and
optionally, wherein each of the CTA-associated MHC class I epitope is selected from the group consisting of: FVQENYLEY, EVDPTSHSY, NTDNNLAVY, EVDPIGHLY, GVYDGREHTV, ALREEGEGV, KVLEYVIKV, GVYDGEEHSV, KLVELEHTL, AETSYVKVL, KVLEHVVRV, EADPTGHSY, SALPTTISF, GVYDGREHTVY, TQHFVQENY, EYVIKVSAR, LVRPSSSGL, LVRPSSSGL, GEMSSNSTAL, TVYGEPRKL, ALAETSYVK, TSYVKVLEH, YPSLREAAL, ALLEEEEGV, GPRQSLQQC, IAYPSLREAAL, and MEVDPIGHL.

Embodiment 32. A composition for delivery of an antigen expression system, comprising:
the antigen expression system,
wherein the antigen expression system comprises one or more vectors,
the one or more vectors comprising (A) a Cancer Testis Antigen (CTA)-encoding nucleic acid sequence encoding a CTA-associated MHC class I epitope and (B) a KRAS-encoding nucleic acid sequence encoding a KRAS-associated MHC class I epitope,
wherein each of the one or more vectors independently comprise:
(a) a vector backbone, wherein the vector backbone comprises a chimpanzee adenovirus vector, optionally wherein the chimpanzee adenovirus vector is a ChAdV68 vector, or an alphavirus vector, optionally wherein the alphavirus vector is a Venezuelan equine encephalitis virus vector; and
(b) a cassette, optionally wherein the cassette is integrated between a native promoter nucleotide sequence native to the vector backbone and a poly(A) sequence, optionally wherein the poly(A) sequence is native to the vector backbone, wherein the cassette comprises:
(i) at least one antigen-encoding nucleic acid sequence, comprising:
(I) at least one epitope-encoding nucleic acid sequence encoding the CTA-associated MHC class I epitope and/or the KRAS-associated MHC class I epitope, optionally comprising at least two distinct epitope-encoding nucleic acid sequences linearly linked to each other, each epitope-encoding nucleic acid sequence optionally comprising:
(A) a MHC class I epitope encoding nucleic acid sequence, wherein the MHC class I epitope encoding nucleic acid sequence encodes a MHC class I epitope 7-15 amino acids in length,
(B) a 5′ linker sequence, wherein the 5′ linker sequence encodes a native N-terminal amino acid sequence of the MHC class I epitope, and wherein the 5′ linker sequence encodes a peptide that is at least 3 amino acids in length,
(C) a 3′ linker sequence, wherein the 3′ linker sequence encodes a native C-terminal acid sequence of the MHC class I epitope, and wherein the 3′ linker sequence encodes a peptide that is at least 3 amino acids in length, and
wherein the cassette is operably linked to the native promoter nucleotide sequence, wherein each of the epitope-encoding nucleic acid sequences encodes a polypeptide that is between 13 and 25 amino acids in length, and wherein each 3′ end of each epitope-encoding nucleic acid sequence is linked to the 5′ end of the following epitope-encoding nucleic acid sequence with the exception of the final epitope-encoding nucleic acid sequence in the cassette; and
(ii) at least two MHC class II epitope-encoding nucleic acid sequences comprising:
(I) a PADRE MHC class II sequence (SEQ ID NO:48),
(II) a Tetanus toxoid MHC class II sequence (SEQ ID NO:46),
(III) a first nucleic acid sequence encoding a GPGPG amino acid linker sequence linking the PADRE MHC class II sequence and the Tetanus toxoid MHC class II sequence,
(IV) a second nucleic acid sequence encoding a GPGPG amino acid linker sequence linking the 5′ end of the at least two MHC class II epitope-encoding nucleic acid sequences to the epitope-encoding nucleic acid sequences,
(V) optionally, a third nucleic acid sequence encoding a GPGPG amino acid linker sequence at the 3′ end of the at least two MHC class II epitope-encoding nucleic acid sequences;
(iii) optionally, a second promoter nucleotide sequence operably linked to the antigen-encoding nucleic acid sequence; and
wherein if the second promoter nucleotide sequence is absent, the antigen-encoding nucleic acid sequence is operably linked to the native promoter nucleotide sequence, and
wherein the at least one antigen-encoding nucleic acid sequence comprises at least two iterations of the epitope-encoding nucleic acid sequence encoding the CTA-associated MHC class I epitope and/or the KRAS-associated MHC class I epitope.

Embodiment 33. A composition for delivery of an antigen expression system, comprising:
the antigen expression system,
wherein the antigen expression system comprises one or more vectors,
the one or more vectors comprising:
(a) a vector backbone, wherein the vector backbone comprises a chimpanzee adenovirus vector, optionally wherein the chimpanzee adenovirus vector is a ChAdV68 vector, or an alphavirus vector, optionally wherein the alphavirus vector is a Venezuelan equine encephalitis virus vector; and
(b) a cassette, optionally wherein the cassette is integrated between a native promoter nucleotide sequence native to the vector backbone and a poly(A) sequence, optionally wherein the poly(A) sequence is native to the vector backbone, wherein the cassette comprises:
(i) at least one antigen-encoding nucleic acid sequence, comprising:
(I) at least one epitope-encoding nucleic acid sequence encoding a CTA-associated MHC class I epitope, optionally comprising at least two distinct epitope-encoding nucleic acid sequences linearly linked to each other, each epitope-encoding nucleic acid sequence optionally comprising:
(A) a MHC class I epitope encoding nucleic acid sequence, wherein the MHC class I epitope encoding nucleic acid sequence encodes a MHC class I epitope 7-15 amino acids in length,
(B) a 5′ linker sequence, wherein the 5′ linker sequence encodes a native N-terminal amino acid sequence of the MHC class I epitope, and wherein the 5′ linker sequence encodes a peptide that is at least 3 amino acids in length,
(C) a 3′ linker sequence, wherein the 3′ linker sequence encodes a native C-terminal acid sequence of the MHC class I epitope, and wherein the 3′ linker sequence encodes a peptide that is at least 3 amino acids in length, and
wherein the cassette is operably linked to the native promoter nucleotide sequence, wherein each of the epitope-encoding nucleic acid sequences encodes a polypeptide that is between 13 and 25 amino acids in length, and wherein each 3′ end of each epitope-encoding nucleic acid sequence is linked to the 5′ end of the following epitope-encoding nucleic acid sequence with the exception of the final epitope-encoding nucleic acid sequence in the cassette; and
(ii) at least two MHC class II epitope-encoding nucleic acid sequences comprising:
(I) a PADRE MHC class II sequence (SEQ ID NO:48),
(II) a Tetanus toxoid MHC class II sequence (SEQ ID NO:46),
(III) a first nucleic acid sequence encoding a GPGPG amino acid linker sequence linking the PADRE MHC class II sequence and the Tetanus toxoid MHC class II sequence,
(IV) a second nucleic acid sequence encoding a GPGPG amino acid linker sequence linking the 5′ end of the at least two MHC class II epitope-encoding nucleic acid sequences to the epitope-encoding nucleic acid sequences,
(V) optionally, a third nucleic acid sequence encoding a GPGPG amino acid linker sequence at the 3′ end of the at least two MHC class II epitope-encoding nucleic acid sequences;
(iii) optionally, a second promoter nucleotide sequence operably linked to the antigen-encoding nucleic acid sequence; and
wherein if the second promoter nucleotide sequence is absent, the antigen-encoding nucleic acid sequence is operably linked to the native promoter nucleotide sequence, and
wherein the at least one antigen-encoding nucleic acid sequence comprises at least two iterations of the epitope-encoding nucleic acid sequence encoding the CTA-associated MHC class I epitope.

Embodiment 34. The composition of any one of embodiments 29-33, wherein an ordered sequence of each element of the cassette is described in the formula, from 5′ to 3′, comprising:

Pa-(L5b-Nc-L3d)X-(G5e-Uf)Y-G3g
wherein,
P comprises the second promoter nucleotide sequence, where a=0 or 1,
N comprises one of the distinct epitope-encoding nucleic acid sequences, where c=1,
L5 comprises the 5′ linker sequence, where b=0 or 1,
L3 comprises the 3′ linker sequence, where d=0 or 1,
G5 comprises one of the at least one nucleic acid sequences encoding a GPGPG amino acid linker, where e=0 or 1,
G3 comprises one of the at least one nucleic acid sequences encoding a GPGPG amino acid linker, where g=0 or 1,
U comprises one of the at least one MHC class II epitope-encoding nucleic acid sequence, where f=1,
X=1 to 400, where for each X the corresponding Nc is an epitope-encoding nucleic acid sequence, and
Y=0, 1, or 2, where for each Y the corresponding Uf is an MHC class II epitope-encoding nucleic acid sequence.

Embodiment 35. The composition of embodiment 34, wherein for each X the corresponding Nc is the epitope-encoding nucleic acid sequence encoding the CTA-associated MHC class I epitope and/or the KRAS-associated MHC class I epitope.

Embodiment 36. The composition of embodiment 34 or 35, wherein for each Y the corresponding Uf is a distinct MHC class II epitope-encoding nucleic acid sequence.

Embodiment 37. The composition of any one of embodiments 34-36, wherein
a=0, b=1, d=1, e=1, g=1, h=1, X=10, Y=2,
the at least one promoter nucleotide sequence is a single native promoter nucleotide sequence native to the vector backbone,
the at least one polyadenylation poly(A) sequence is a poly(A) sequence of at least 100 consecutive A nucleotides provided by the vector backbone,
each N encodes an epitope 7-15 amino acids in length,
L5 is a native 5′ linker sequence that encodes a native N-terminal amino acid sequence of the epitope, and wherein the 5′ linker sequence encodes a peptide that is at least 3 amino acids in length,
L3 is a native 3′ linker sequence that encodes a native C-terminal amino acid sequence of the epitope, and wherein the 3′ linker sequence encodes a peptide that is at least 3 amino acids in length,
U is each of a PADRE class II sequence and a Tetanus toxoid MHC class II sequence,
the vector backbone comprises a chimpanzee adenovirus vector, optionally wherein the chimpanzee adenovirus vector is a ChAdV68 vector, or an alphavirus vector, optionally wherein the alphavirus vector is a Venezuelan equine encephalitis virus vector, optionally wherein the native promoter nucleotide sequence is a 26S promoter when the vector backbone comprises an alphavirus vector, and
each of the MHC class II epitope-encoding nucleic acid sequences encodes a polypeptide that is between 13 and 25 amino acids in length.

Embodiment 38. The composition of any one of embodiments 29-37, wherein the at least two iterations is at least 3, at least 4, at least 5, at least 6, at least 7, or at least 8 iterations.

Embodiment 39. The composition of any one of embodiments 29-37, wherein the at least two iterations is at least 8 iterations.

Embodiment 40. The composition of any one of embodiments 29-37, wherein the at least two iterations is between 2-3, between 2-4, between 2-5, between 2-6, between 2-7, or between 2-8 iterations.

Embodiment 41. The composition of any one of embodiments 34-37, wherein the at least two iterations is 7 iterations or less, 6 iterations or less, 5 iterations or less, 4 iterations or less, or 3 iterations or less.

Embodiment 42. The composition of any one of embodiments 29-41, wherein the at least one antigen-encoding nucleic acid sequence comprises at least two iterations of at least two distinct epitope-encoding nucleic acid sequences.

Embodiment 43. The composition of any one of embodiments 29-41, wherein the at least one antigen-encoding nucleic acid sequence comprises at least two iterations of at least 3, at least 4, at least 5, at least 6, at least 7, at least 8, at least 9, or at least 10 distinct epitope-encoding nucleic acid sequences.

Embodiment 44. The composition of any one of embodiments 29-43, wherein the at least two iterations are separated by at least one separate distinct epitope-encoding nucleic acid sequence.

Embodiment 45. The composition of any one of embodiments 29-43, wherein the at least two iterations are separated by at least 2 separate distinct epitope-encoding nucleic acid sequences.

Embodiment 46. The composition of any one of embodiments 29-43, wherein the at least two iterations, inclusive of the optional 5′ linker sequence and/or the optional 3′ linker sequence, are separated by at least 75 nucleotides.

Embodiment 47. The composition of any one of embodiments 29-43, wherein the at least two iterations, inclusive of the optional 5′ linker sequence and/or the optional 3′ linker sequence, are separated by at least 150 nucleotides, at least 300 nucleotides, or at least 675 nucleotides.

Embodiment 48. The composition of any one of embodiments 29-43, wherein the at least two iterations, inclusive of the optional 5′ linker sequence and/or the optional 3′ linker sequence, are separated by at least 50 nucleotides, at least 100 nucleotides, at least 200 nucleotides, at least 250 nucleotides, at least 350 nucleotides, at least 400 nucleotides, at least 450 nucleotides, at least 500 nucleotides, at least 700 nucleotides, at least 700 nucleotides, at least 750 nucleotides, at least 800 nucleotides, at least 900 nucleotides, or at least 1000 nucleotides.

Embodiment 49. The composition of any one of embodiments 29-43, wherein the at least two iterations, inclusive of the optional 5′ linker sequence and/or the optional 3′ linker sequence, are separated by at least 10 nucleotides, at least 15 nucleotides, at least 20 nucleotides, at least 25 nucleotides, at least 30 nucleotides, at least 35 nucleotides, at least 40 nucleotides, at least 45 nucleotides, at least 50 nucleotides, at least 55 nucleotides, at least 60 nucleotides, at least 65 nucleotides, or at least 70 nucleotides.

Embodiment 50. The composition of any one of embodiments 29-49, wherein the at least one antigen-encoding nucleic acid sequence is described, from 5′ to 3′, by the formula:

(Ex-(ENn)y)z
wherein E represents a nucleotide sequence comprising a distinct epitope-encoding nucleic acid sequence,
n represents the number of separate distinct epitope-encoding nucleic acid sequences and is any integer including 0,
EN represents a nucleotide sequence comprising the separate distinct epitope-encoding nucleic acid sequence for each corresponding n,
for each iteration of z: x=0 or 1, y=0 or 1 for each n, and at least one of x or y=1, and
z=1 or greater, optionally wherein the antigen-encoding nucleic acid sequence comprises at least two iterations of E, a given EN, or a combination thereof, and
at least one of the distinct epitope-encoding nucleic acid sequences encodes a distinct CTA-associated MHC class I epitope and/or a distinct KRAS-associated MHC class I epitope, optionally wherein at least one of the distinct epitope-encoding nucleic acid sequences encoding the CTA-associated MHC class I epitope and/or the KRAS-associated MHC class I epitope comprises at least two iterations.

Embodiment 51. The composition of any one of embodiments 29-50, wherein the CTA-associated MHC class I epitope is selected from the group consisting of: a MAGEA1 MHC class I epitope encoding nucleic acid sequence, a MAGEA3 MHC class I epitope encoding nucleic acid sequence, a MAGEA4 MHC class I epitope encoding nucleic acid sequence, a MAGEA6 MHC class I epitope encoding nucleic acid sequence, a MAGEA8 MHC class I epitope encoding nucleic acid sequence, a MAGEA11 MHC class I epitope encoding nucleic acid sequence, a MAGEB2 MHC class I epitope encoding nucleic acid sequence, a CTCFL MHC class I epitope encoding nucleic acid sequence, and a CT83 MHC class I epitope encoding nucleic acid sequence.

Embodiment 52. The composition of embodiment 51, wherein the CTA-associated MHC class I epitope is selected from the group consisting of: FVQENYLEY, EVDPTSHSY, NTDNNLAVY, EVDPIGHLY, GVYDGREHTV, ALREEGEGV, KVLEYVIKV, GVYDGEEHSV, KLVELEHTL, AETSYVKVL, KVLEHVVRV, EADPTGHSY, SALPTTISF, GVYDGREHTVY, TQHFVQENY, EYVIKVSAR, LVRPSSSGL, LVRPSSSGL, GEMSSNSTAL, TVYGEPRKL, ALAETSYVK, TSYVKVLEH, YPSLREAAL, ALLEEEEGV, GPRQSLQQC, IAYPSLREAAL, and MEVDPIGHL.

Embodiment 53. The composition of any one of embodiments 29-50, wherein CTA-encoding nucleic acid sequence encodes:
each of a MAGEA1 MHC class I epitope encoding nucleic acid sequence, a MAGEA4 MHC class I epitope encoding nucleic acid sequence, a MAGEB2 MHC class I epitope encoding nucleic acid sequence, a MAGEA11 MHC class I epitope encoding nucleic acid sequence, a MAGEA3 MHC class I epitope encoding nucleic acid sequence, and a CT83 MHC class I epitope encoding nucleic acid sequence,
each of a MAGEA6 MHC class I epitope encoding nucleic acid sequence, a MAGEA11 MHC class I epitope encoding nucleic acid sequence, a MAGEA3 MHC class I epitope encoding nucleic acid sequence, and a CT83 MHC class I epitope encoding nucleic acid sequence,
each of a MAGEA1 MHC class I epitope encoding nucleic acid sequence, a MAGEA4 MHC class I epitope encoding nucleic acid sequence, a MAGEB2 MHC class I epitope encoding nucleic acid sequence, a MAGEA11 MHC class I epitope encoding nucleic acid sequence, and a CT83 MHC class I epitope encoding nucleic acid sequence, or
each of a MAGEA3 MHC class I epitope encoding nucleic acid sequence, a MAGEA4 MHC class I epitope encoding nucleic acid sequence, a MAGEB2 MHC class I epitope encoding nucleic acid sequence, a MAGEA11 MHC class I epitope encoding nucleic acid sequence, and a CT83 MHC class I epitope encoding nucleic acid sequence.

Embodiment 54. The composition of any one of embodiments 29-53, wherein one or more of the nucleic acid sequences encoding the CTA-associated MHC class I epitope and/or the KRAS-associated MHC class I epitope comprises at least 2, at least 3, at least 4, at least 5, at least 6, or at least 7 iterations.

Embodiment 55. The composition of any one of embodiments 29-53, wherein each of the nucleic acid sequences encoding the CTA-associated MHC class I epitope and/or the KRAS-associated MHC class I epitope comprises at least 2, at least 3, at least 4, at least 5, at least 6, or at least 7 iterations.

Embodiment 56. The composition of any one of embodiments 29-53, wherein one or more of the nucleic acid sequences encoding the CTA-associated MHC class I epitope and/or the KRAS-associated MHC class I epitope comprises at least 4 iterations.

Embodiment 57. The composition of any one of embodiments 29-53, wherein each of the nucleic acid sequences encoding the CTA-associated MHC class I epitope and/or the KRAS-associated MHC class I epitope comprises at least 4 iterations.

Embodiment 58. The composition of any one of embodiments 29-57, wherein each of the CTA-associated MHC class I epitopes are selected from the group consisting of: FVQENYLEY, EVDPTSHSY, NTDNNLAVY, EVDPIGHLY, GVYDGREHTV, ALREEGEGV, KVLEYVIKV, GVYDGEEHSV, KLVELEHTL, AETSYVKVL, KVLEHVVRV, EADPTGHSY, SALPTTISF, GVYDGREHTVY, TQHFVQENY, EYVIKVSAR, LVRPSSSGL, and GEMSSNSTAL.

Embodiment 59. The composition of any one of embodiments 29-58, wherein at least one of the distinct epitope-encoding nucleic acid sequences encodes a distinct KRAS-associated MHC class I neoepitope.

Embodiment 60. The composition of embodiment 59, wherein one or more of the distinct KRAS-associated MHC class I neoepitopes independently comprises a KRAS G12C mutation, a KRAS G12V mutation, a KRAS G12D mutation, or a KRAS Q61H mutation.

Embodiment 61. The composition of embodiment 59, wherein one or more of the distinct KRAS-associated MHC class I neoepitopes independently comprises a KRAS G12C mutation or a KRAS G12V mutation.

Embodiment 62. The composition of embodiment 59, wherein one or more of the distinct KRAS-associated MHC class I neoepitopes independently comprises each of a KRAS G12C mutation and a KRAS G12V mutation.

Embodiment 63. The composition of any one of embodiments 59-62, wherein the distinct KRAS-associated MHC class I neoepitope comprising the KRAS G12C mutation is selected from the group consisting of KLVVVGACGV, VVVGACGVGK, GACGVGKSAL, and combinations thereof.

Embodiment 64. The composition of any one of embodiments 59-63, wherein the distinct KRAS-associated MHC class I neoepitope comprising the KRAS G12V mutation is selected from the group consisting of KLVVVGAVGV, VVVGAVGVGK, AVGVGKSAL, GAVGVGKSAL, and combinations thereof.

Embodiment 65. The composition of any one of embodiments 59-64, wherein at least one of the distinct epitope-encoding nucleic acid sequences encoding the distinct KRAS-associated MHC class I neoepitope comprises at least two iterations of the distinct epitope-encoding nucleic acid sequences encoding the distinct KRAS-associated MHC class I neoepitope.

Embodiment 66. The composition of any one of embodiments 59-65, wherein each of the distinct epitope-encoding nucleic acid sequences encoding the distinct KRAS-associated MHC class I neoepitope comprises at least two iterations of the distinct epitope-encoding nucleic acid sequences encoding the distinct KRAS-associated MHC class I neoepitope.

Embodiment 67. The composition of any one of the above embodiments, wherein the at least two iterations comprises a number of iterations, or z comprises a number, sufficient to stimulate a greater immune response relative to an antigen-encoding nucleic acid sequence comprising a single iteration of the at least one epitope-encoding nucleic acid sequence.

Embodiment 68. The composition of any one of the above embodiments, wherein the at least two iterations comprises a number of iterations, or z comprises a number, sufficient to stimulate an immune response, and a single iteration of the at least one epitope-encoding nucleic acid sequence is insufficient to stimulate the immune response or insufficient to stimulate a detectable immune response.

Embodiment 69. The composition of embodiments 67 or 68, wherein the immune response is an expansion of epitope-specific T cells following in vivo immunization with the composition for delivery of the antigen expression system.

Embodiment 70. The composition of embodiments 67 or 68, wherein the immune response is increased activation of epitope-specific T cells and/or increased epitope-specific killing by epitope-specific T cells following in vivo immunization with the composition for delivery of the antigen expression system.

Embodiment 71. The composition of any one of the above embodiments, wherein the composition further comprises a nanoparticulate delivery vehicle.

Embodiment 72. The composition of embodiment 71, wherein the nanoparticulate delivery vehicle is a lipid nanoparticle (LNP).

Embodiment 73. The composition of embodiment 72, wherein the LNP comprises ionizable amino lipids.

Embodiment 74. The composition of embodiment 73, wherein the ionizable amino lipids comprise MC3-like (dilinoleylmethyl-4-dimethylaminobutyrate) molecules.

Embodiment 75. The composition of any of embodiments 71-74, wherein the nanoparticulate delivery vehicle encapsulates the antigen expression system.

Embodiment 76. The composition of any of embodiments 71-75, wherein the CTA-encoding nucleic acid sequence and the KRAS-encoding nucleic acid sequence are formulated in the same nanoparticulate delivery vehicle.

Embodiment 77. The composition of any of embodiments 71-75, wherein the CTA-encoding nucleic acid sequence and the KRAS-encoding nucleic acid sequence are formulated in separate nanoparticulate delivery vehicles, and wherein the composition comprises a mixture of the separate nanoparticulate delivery vehicles.

Embodiment 78. The composition of any one of the above embodiments, wherein the CTA-encoding nucleic acid sequence and the KRAS-encoding nucleic acid sequence are encoded in the same cassette.

Embodiment 79. The composition of embodiment 78, wherein the CTA-encoding nucleic acid sequence and the KRAS-encoding nucleic acid sequence are linked together by a 2A ribosome skipping sequence.

Embodiment 80. The composition of embodiment 78, wherein the CTA-encoding nucleic acid sequence and the KRAS-encoding nucleic acid sequence are each independently operably linked to a separate promoter.

Embodiment 81. The composition of any one of the above embodiments, wherein the CTA-encoding nucleic acid sequence and the KRAS-encoding nucleic acid sequence are encoded on separate vectors.

Embodiment 82. The composition of any one of the above embodiments, wherein the cassette is integrated between the at least one promoter nucleotide sequence and the at least one poly(A) sequence.

Embodiment 83. The composition of any one of the above embodiments, wherein the second promoter is absent and the at least one promoter nucleotide sequence is operably linked to the antigen-encoding nucleic acid sequence.

Embodiment 84. The composition of any one of the above embodiments, wherein the one or more vectors comprise one or more +-stranded RNA vectors.

Embodiment 85. The composition of embodiment 84 wherein the one or more +-stranded RNA vectors comprise a 5′ 7-methylguanosine (m7 g) cap.

Embodiment 86. The composition of embodiment 84 or 85, wherein the one or more +-stranded RNA vectors are produced by in vitro transcription.

Embodiment 87. The composition of any one of the above embodiments, wherein the one or more vectors are self-replicating within a mammalian cell.

Embodiment 88. The composition of any one of the above embodiments, wherein the backbone comprises at least one nucleotide sequence of an Aura virus, a Fort Morgan virus, a Venezuelan equine encephalitis virus, a Ross River virus, a Semliki Forest virus, a Sindbis virus, or a Mayaro virus.

Embodiment 89. The composition of any one of the above embodiments, wherein the backbone comprises at least one nucleotide sequence of a Venezuelan equine encephalitis virus.

Embodiment 90. The composition of embodiment 88 or 89, wherein the backbone comprises at least sequences for nonstructural protein-mediated amplification, a 26S promoter sequence, a poly(A) sequence, a nonstructural protein 1 (nsP1) gene, a nsP2 gene, a nsP3 gene, and a nsP4 gene encoded by the nucleotide sequence of the Aura virus, the Fort Morgan virus, the Venezuelan equine encephalitis virus, the Ross River virus, the Semliki Forest virus, the Sindbis virus, or the Mayaro virus.

Embodiment 91. The composition of embodiment 88 or 89, wherein the backbone comprises at least sequences for nonstructural protein-mediated amplification, a 26S promoter sequence, and a poly(A) sequence encoded by the nucleotide sequence of the Aura virus, the Fort Morgan virus, the Venezuelan equine encephalitis virus, the Ross River virus, the Semliki Forest virus, the Sindbis virus, or the Mayaro virus.

Embodiment 92. The composition of embodiment 90 or 91, wherein sequences for nonstructural protein-mediated amplification are selected from the group consisting of: an alphavirus 5′ UTR, a 51-nt CSE, a 24-nt CSE, a 26S subgenomic promoter sequence, a 19-nt CSE, an alphavirus 3′ UTR, or combinations thereof.

Embodiment 93. The composition of any one of embodiments 90-92, wherein the backbone does not encode structural virion proteins capsid, E2 and E1.

Embodiment 94. The composition of embodiment 93, wherein the cassette is inserted in place of structural virion proteins within the nucleotide sequence of the Aura virus, the Fort Morgan virus, the Venezuelan equine encephalitis virus, the Ross River virus, the Semliki Forest virus, the Sindbis virus, or the Mayaro virus.

Embodiment 95. The composition of embodiment 88 or 89, wherein the Venezuelan equine encephalitis virus comprises the sequence of SEQ ID NO:3 or SEQ ID NO:5.

Embodiment 96. The composition of embodiment 88 or 89, wherein the Venezuelan equine encephalitis virus comprises the sequence of SEQ ID NO:3 or SEQ ID NO:5 further comprising a deletion between base pair 7544 and 11175.

Embodiment 97. The composition of embodiment 96, wherein the backbone comprises the sequence set forth in SEQ ID NO:6 or SEQ ID NO:7.

Embodiment 98. The composition of embodiment 96 or 97, wherein the cassette is inserted at position 7544 to replace the deletion between base pairs 7544 and 11175 as set forth in the sequence of SEQ ID NO:3 or SEQ ID NO:5.

Embodiment 99. The composition of embodiment 94-98, wherein the insertion of the cassette provides for transcription of a polycistronic RNA comprising the nsP1-4 genes and the at least one antigen-encoding nucleic acid sequence, wherein the nsP1-4 genes and the at least one antigen-encoding nucleic acid sequence are in separate open reading frames.

Embodiment 100. The composition of any one of the above embodiments, wherein the backbone comprises at least one nucleotide sequence of a chimpanzee adenovirus vector.

Embodiment 101. The composition of embodiment 100, wherein the chimpanzee adenovirus vector is a ChAdV68 vector, optionally wherein the ChAdV68 vector comprises a ChAdV68 vector backbone comprising:
the sequence set forth in SEQ ID NO:1;
the sequence set forth in SEQ ID NO:1, except that the sequence is fully deleted or functionally deleted in at least one gene selected from the group consisting of the chimpanzee adenovirus E1A, E1B, E2A, E2B, E3, E4, L1, L2, L3, L4, and L5 genes of the sequence set forth in SEQ ID NO:1, optionally wherein the sequence is fully deleted or functionally deleted in: (1) E1A and E1B; (2) E1A, E1B, and E3; or (3) E1A, E1B, E3, and E4 of the sequence set forth in SEQ ID NO:1;
a gene or regulatory sequence obtained from the sequence of SEQ ID NO:1, optionally wherein the gene is selected from the group consisting of the chimpanzee adenovirus inverted terminal repeat (ITR), E1A, E1B, E2A, E2B, E3, E4, L1, L2, L3, L4, and L5 genes of the sequence set forth in SEQ ID NO:1;
a partially deleted E4 gene comprising a deleted or partially-deleted E4orf2 region and a deleted or partially-deleted E4orf3 region, and optionally a deleted or partially-deleted E4orf4 region;
at least nucleotides 2 to 36,518 of the sequence set forth in SEQ ID NO:1 and further comprising: (1) an E1 deletion of at least nucleotides 577 to 3403 of the sequence shown in SEQ ID NO:1, (2) an E3 deletion of at least nucleotides 27,125 to 31,825 of the sequence shown in SEQ ID NO:1, and (3) an E4 deletion of at least nucleotides 34,916 to 35,642 of the sequence shown in SEQ ID NO:1; optionally wherein the antigen cassette is inserted within the E1 deletion;
one or more deletions between base pair number 577 and 3403 or between base pair 456 and 3014, and optionally wherein the vector further comprises one or more deletions between base pair 27,125 and 31,825 or between base pair 27,816 and 31,333 of the sequence set forth in SEQ ID NO:1; or
one or more deletions between base pair number 3957 and 10346, base pair number 21787 and 23370, and base pair number 33486 and 36193 of the sequence set forth in SEQ ID NO:1, and
optionally wherein the cassette is inserted in the ChAdV vector backbone at the E1 region, E3 region, and/or any deleted AdV region that allows incorporation of the cassette.

Embodiment 102. The composition of any one of the above embodiments, wherein the at least one promoter nucleotide sequence is the native 26S promoter nucleotide sequence encoded by the backbone.

Embodiment 103. The composition of any one of the above embodiments, wherein the at least one promoter nucleotide sequence is an exogenous RNA promoter.

Embodiment 104. The composition of any one of the above embodiments, wherein the second promoter nucleotide sequence is a 26S promoter nucleotide sequence.

Embodiment 105. The composition of any one of the above embodiments, wherein the second promoter nucleotide sequence comprises multiple 26S promoter nucleotide sequences, wherein each 26S promoter nucleotide sequence provides for transcription of one or more of the separate open reading frames.

Embodiment 106. The composition of any one of the above embodiments, wherein one or more of the cassettes are at least 100, 200, 300, 400, 500, 600, 700, 800, or 900 nucleotides in length.

Embodiment 107. The composition of any one of the above embodiments, wherein one or more of the cassettes are at least 1000, 2000, 3000, 4000, 5000, 6000, 7000, 8000, 9000, or 10000 nucleotides in length.

Embodiment 108. The composition of any one of the above embodiments, wherein one or more of the cassettes is at least 3500 nucleotides in length.

Embodiment 109. The composition of any one of the above embodiments, wherein one or more of the cassettes is at least 6000 nucleotides in length.

Embodiment 110. The composition of any one of the above embodiments, wherein at least one of the at least one antigen-encoding nucleic acid sequences encodes a polypeptide sequence or portion thereof that is presented by MHC class I on a cell surface, optionally a tumor cell surface.

Embodiment 111. The composition of any one of the above embodiments, wherein each epitope-encoding nucleic acid sequence is linked directly to one another.

Embodiment 112. The composition of any one of the above embodiments, wherein at least one of the at least one epitope-encoding nucleic acid sequences is linked to a distinct epitope-encoding nucleic acid sequence with a nucleic acid sequence encoding a linker.

Embodiment 113. The composition of embodiment 112, wherein the linker links two MHC class I sequences or an MHC class I sequence to an MHC class II sequence.

Embodiment 114. The composition of embodiment 113, wherein the linker is selected from the group consisting of: (1) consecutive glycine residues, at least 2, 3, 4, 5, 6, 7, 8, 9, or 10 residues in length; (2) consecutive alanine residues, at least 2, 3, 4, 5, 6, 7, 8, 9, or 10 residues in length; (3) two arginine residues (RR); (4) alanine, alanine, tyrosine (AAY); (5) a consensus sequence at least 2, 3, 4, 5, 6, 7, 8, 9, or 10 amino acid residues in length that is processed efficiently by a mammalian proteasome; and (6) one or more native sequences flanking the antigen derived from the cognate protein of origin and that is at least 2, 3, 4, 5, 6, 7, 8, 9, 10, 11, 12, 13, 14, 15, 16, 17, 18, 19, 20, or 2-20 amino acid residues in length.

Embodiment 115. The composition of embodiment 112, wherein the linker links two MHC class II sequences or an MHC class II sequence to an MHC class I sequence.

Embodiment 116. The composition of embodiment 115, wherein the linker comprises the sequence GPGPG.

Embodiment 117. The composition of any one of the above embodiments, wherein at least one sequence of the at least one epitope-encoding nucleic acid sequences is linked, operably or directly, to a separate or contiguous sequence that enhances the expression, stability, cell trafficking, processing and presentation, and/or immunogenicity of the at least one epitope-encoding nucleic acid sequences of epitope encoded therefrom.

Embodiment 118. The composition of embodiment 117, wherein the separate or contiguous sequence comprises at least one of: a ubiquitin sequence, a ubiquitin sequence modified to increase proteasome targeting (e.g., the ubiquitin sequence contains a Gly to Ala substitution at position 76), an immunoglobulin signal sequence (e.g., IgK), a major histocompatibility class I sequence, lysosomal-associated membrane protein (LAMP)-1, human dendritic cell lysosomal-associated membrane protein, and a major histocompatibility class II sequence; optionally wherein the ubiquitin sequence modified to increase proteasome targeting is A76.

Embodiment 119. The composition of any one of the above embodiments, wherein at least one of the at least one epitope-encoding nucleic acid sequences encodes a polypeptide sequence or portion thereof that has increased binding affinity to its corresponding MHC allele relative to the translated, corresponding wild-type nucleic acid sequence.

Embodiment 120. The composition of any one of the above embodiments, wherein at least one of the at least one epitope-encoding nucleic acid sequences encodes a polypeptide sequence or portion thereof that has increased binding stability to its corresponding MHC allele relative to the translated, corresponding wild-type nucleic acid sequence.

Embodiment 121. The composition of any one of the above embodiments, wherein at least one of the at least one epitope-encoding nucleic acid sequences encodes a polypeptide sequence or portion thereof that has an increased likelihood of presentation on its corresponding MHC allele relative to the translated, corresponding wild-type nucleic acid sequence.

Embodiment 122. The composition of any one of the above embodiments, wherein the at least one alteration comprises a point mutation, a frameshift mutation, a non-frameshift mutation, a deletion mutation, an insertion mutation, a splice variant, a genomic rearrangement, or a proteasome-generated spliced antigen.

Embodiment 123. The composition of any one of the above embodiments, wherein the tumor is selected from the group consisting of: lung cancer, melanoma, breast cancer, ovarian cancer, prostate cancer, kidney cancer, gastric cancer, colon cancer, testicular cancer, head and neck cancer, pancreatic cancer, bladder cancer, brain cancer, B-cell lymphoma, acute myelogenous leukemia, adult acute lymphoblastic leukemia, chronic myelogenous leukemia, chronic lymphocytic leukemia, T cell lymphocytic leukemia, non-small cell lung cancer, and small cell lung cancer.

Embodiment 124. The composition of any one of the above embodiments, wherein the tumor is a lung adenocarcinoma, ovarian serous cystadenocarcinoma, lung squamous cell carcinoma, colon cancer, or head and neck squamous cell carcinoma.

Embodiment 125. The composition of any one the above embodiments, wherein the at least one antigen-encoding nucleic acid sequence comprises at least 2-10, 2, 3, 4, 5, 6, 7, 8, 9, or 10 epitope-encoding nucleic acid sequences.

Embodiment 126. The composition of any one of the above embodiments, wherein the at least one antigen-encoding nucleic acid sequence comprises at least 11-20, 15-20, 11-100, 11-200, 11-300, 11-400, 11, 12, 13, 14, 15, 16, 17, 18, 19, 20 or up to 400 epitope-encoding nucleic acid sequences.

Embodiment 127. The composition of any one of the above embodiments, wherein the at least one antigen-encoding nucleic acid sequence comprises at least 2-400 epitope-encoding nucleic acid sequences and wherein at least two of the epitope-encoding nucleic acid sequences encode polypeptide sequences or portions thereof that are presented by MHC class I on a cell surface, optionally a tumor cell surface.

Embodiment 128. The composition of any one the above embodiments, wherein the at least one antigen-encoding nucleic acid sequence comprises at least 2-10, 2, 3, 4, 5, 6, 7, 8, 9, or 10 antigen-encoding nucleic acid sequences.

Embodiment 129. The composition of any one of the above embodiments, wherein the at least one antigen-encoding nucleic acid sequence comprises at least 11-20, 15-20, 11-100, 11-200, 11-300, 11-400, 11, 12, 13, 14, 15, 16, 17, 18, 19, 20 or up to 400 antigen-encoding nucleic acid sequences.

Embodiment 130. The composition of any one of the above embodiments, wherein the at least one antigen-encoding nucleic acid sequence comprises at least 2-400 antigen-encoding nucleic acid sequences and wherein at least two of the antigen-encoding nucleic acid sequences encode polypeptide sequences or portions thereof that are presented by MHC class I on a cell surface, optionally a tumor cell surface.

Embodiment 131. The composition of any one of the above embodiments, wherein when administered to the subject and translated, the CTA-associated MHC class I epitope and/or the KRAS-associated MHC class I epitope is presented on antigen presenting cells resulting in an immune response targeting CTA-associated MHC class I epitope and/or the KRAS-associated MHC class I epitope on the tumor cell surface.

Embodiment 132. The composition of any one of the above embodiments, wherein the at least one antigen-encoding nucleic acid sequences when administered to the subject and translated, CTA-associated MHC class I epitope and/or the KRAS-associated MHC class I epitope is presented on antigen presenting cells resulting in an immune response targeting CTA-associated MHC class I epitope and/or the KRAS-associated MHC class I epitope a tumor cell surface, and optionally wherein the expression of each of the at least one antigen-encoding nucleic acid sequences is driven by the at least one promoter nucleotide sequence.

Embodiment 133. The composition of any one of the above embodiments, wherein each epitope-encoding nucleic acid sequence encodes a polypeptide sequence between 8 and 35 amino acids in length, optionally 9-17, 9-25, 8, 9, 10, 11, 12, 13, 14, 15, 16, 17, 18, 19, 20, 21, 22, 23, 24, 25, 26, 27, 28, 29, 30, 31, 32, 33, 34 or 35 amino acids in length.

Embodiment 134. The composition of any one of the above embodiments, wherein the at least one MHC class II epitope-encoding nucleic acid sequence is present.

Embodiment 135. The composition of any one of the above embodiments, wherein the at least one MHC class II epitope-encoding nucleic acid sequence is present and comprises at least one MHC class II epitope-encoding nucleic acid sequence that comprises at least one alteration that makes the encoded peptide sequence distinct from the corresponding peptide sequence encoded by a wild-type nucleic acid sequence.

Embodiment 136. The composition of any one of the above embodiments, wherein the at least one MHC class II epitope-encoding nucleic acid sequence is 12-20, 12, 13, 14, 15, 16, 17, 18, 19, 20, or 20-40 amino acids in length.

Embodiment 137. The composition of any one of the above embodiments, wherein the at least one MHC class II epitope-encoding nucleic acid sequence is present and comprises at least one universal MHC class II antigen-encoding nucleic acid sequence, optionally wherein the at least one universal sequence comprises at least one of Tetanus toxoid and PADRE.

Embodiment 138. The composition of any one of the above embodiments, wherein the at least one promoter nucleotide sequence or the second promoter nucleotide sequence is inducible.

Embodiment 139. The composition of any one of the above embodiments, wherein the at least one promoter nucleotide sequence or the second promoter nucleotide sequence is non-inducible.

Embodiment 140. The composition of any one of the above embodiments, wherein the at least one poly(A) sequence comprises a poly(A) sequence native to the backbone.

Embodiment 141. The composition of any one of the above embodiments, wherein the at least one poly(A) sequence comprises a poly(A) sequence exogenous to the backbone.

Embodiment 142. The composition of any one the above embodiments, wherein the at least one poly(A) sequence is operably linked to at least one of the at least one antigen-encoding nucleic acid sequences.

Embodiment 143. The composition of any one of the above embodiments, wherein the at least one poly(A) sequence is at least 20, at least 30, at least 40, at least 50, at least 60, at least 70, at least 80, or at least 90 consecutive A nucleotides.

Embodiment 144. The composition of any one of the above embodiments, wherein the at least one poly(A) sequence is at least 100 consecutive A nucleotides.

Embodiment 145. The composition of any one of the above embodiments, wherein the cassette further comprises at least one of: an intron sequence, a woodchuck hepatitis virus posttranscriptional regulatory element (WPRE) sequence, an internal ribosome entry sequence (IRES) sequence, a nucleotide sequence encoding a 2A self-cleaving peptide sequence, a nucleotide sequence encoding a Furin cleavage site, or a sequence in the 5′ or 3′ non-coding region known to enhance the nuclear export, stability, or translation efficiency of mRNA that is operably linked to at least one of the at least one antigen-encoding nucleic acid sequences.

Embodiment 146. The composition of any one of the above embodiments, wherein the cassette further comprises a reporter gene, including but not limited to, green fluorescent protein (GFP), a GFP variant, secreted alkaline phosphatase, luciferase, a luciferase variant, or a detectable peptide or epitope.

Embodiment 147. The composition of embodiment 146, wherein the detectable peptide or epitope is selected from the group consisting of an HA tag, a Flag tag, a His-tag, or a V5 tag.

Embodiment 148. The composition of any one of the above embodiments, wherein the one or more vectors further comprises one or more nucleic acid sequences encoding at least one immune modulator.

Embodiment 149. The composition of embodiment 148, wherein the immune modulator is an anti-CTLA4 antibody or an antigen-binding fragment thereof, an anti-PD-1 antibody or an antigen-binding fragment thereof, an anti-PD-L1 antibody or an antigen-binding fragment thereof, an anti-4-1BB antibody or an antigen-binding fragment thereof, or an anti-OX-40 antibody or an antigen-binding fragment thereof.

Embodiment 150. The composition of embodiment 149, wherein the antibody or antigen-binding fragment thereof is a Fab fragment, a Fab′ fragment, a single chain Fv (scFv), a single domain antibody (sdAb) either as single specific or multiple specificities linked together (e.g., camelid antibody domains), or full-length single-chain antibody (e.g., full-length IgG with heavy and light chains linked by a flexible linker).

Embodiment 151. The composition of embodiment 149 or 150, wherein the heavy and light chain sequences of the antibody are a contiguous sequence separated by either a self-cleaving sequence such as 2A or IRES; or the heavy and light chain sequences of the antibody are linked by a flexible linker such as consecutive glycine residues.

Embodiment 152. The composition of embodiment 148, wherein the immune modulator is a cytokine.

Embodiment 153. The composition of embodiment 152, wherein the cytokine is at least one of IL-2, IL-7, IL-12, IL-15, or IL-21 or variants thereof of each.

Embodiment 154. The composition of any one of the above embodiments, wherein at least one epitope-encoding nucleic acid sequence is selected by performing the steps of:
(a) obtaining at least one of exome, transcriptome, or whole genome nucleotide sequencing data from a tumor, wherein the nucleotide sequencing data is used to obtain data representing peptide sequences of each of a set of antigens;
(b) inputting the peptide sequence of each antigen into a presentation model to generate a set of numerical likelihoods that each of the antigens is presented by one or more of the MHC alleles on a cell surface, optionally a tumor cell surface, the set of numerical likelihoods having been identified at least based on received mass spectrometry data; and
(c) selecting a subset of the set of antigens based on the set of numerical likelihoods to generate a set of selected antigens which are used to generate the at least one epitope-encoding nucleic acid sequence.

Embodiment 155. The composition of embodiment 154, wherein a number of the set of selected antigens is 2-20.

Embodiment 156. The composition of embodiment 154 or 155, wherein the presentation model represents dependence between:
(a) presence of a pair of a particular one of the MHC alleles and a particular amino acid at a particular position of a peptide sequence; and
(b) likelihood of presentation on a cell surface, optionally a tumor cell surface, by the particular one of the MHC alleles of the pair, of such a peptide sequence comprising the particular amino acid at the particular position.

Embodiment 157. The composition of embodiment 154-156, wherein selecting the set of selected antigens comprises selecting antigens that have an increased likelihood of being presented on the cell surface relative to unselected antigens based on the presentation model, optionally wherein the selected antigens have been validated as being presented by one or more specific HLA alleles.

Embodiment 158. The composition of embodiment 154-157, wherein selecting the set of selected antigens comprises selecting antigens that have an increased likelihood of being capable of inducing a tumor-specific or infectious disease-specific immune response in the subject relative to unselected antigens based on the presentation model.

Embodiment 159. The composition of embodiment 154-158, wherein selecting the set of selected antigens comprises selecting antigens that have an increased likelihood of being capable of being presented to naïve T cells by professional antigen presenting cells (APCs) relative to unselected antigens based on the presentation model, optionally wherein the APC is a dendritic cell (DC).

Embodiment 160. The composition of embodiment 154-159, wherein selecting the set of selected antigens comprises selecting antigens that have a decreased likelihood of being subject to inhibition via central or peripheral tolerance relative to unselected antigens based on the presentation model.

Embodiment 161. The composition of embodiment 154-160, wherein selecting the set of selected antigens comprises selecting antigens that have a decreased likelihood of being capable of inducing an autoimmune response to normal tissue in the subject relative to unselected antigens based on the presentation model.

Embodiment 162. The composition of embodiment 154-161, wherein exome or transcriptome nucleotide sequencing data is obtained by performing sequencing on a tumor cell or tissue, an infected cell, or an infectious disease organism.

Embodiment 163. The composition of embodiment 162, wherein the sequencing is next generation sequencing (NGS) or any massively parallel sequencing approach.

Embodiment 164. The composition of any one of the above embodiments, wherein the cassette comprises junctional epitope sequences formed by adjacent sequences in the cassette.

Embodiment 165. The composition of embodiment 164, wherein at least one or each junctional epitope sequence has an affinity of greater than 500 nM for MHC.

Embodiment 166. The composition of embodiments 164 or 165, wherein each junctional epitope sequence is non-self.

Embodiment 167. The composition of any one of the above embodiments, wherein each of the MHC class I epitopes is predicted or validated to be capable of presentation by at least one HLA allele present in at least 5% of a population.

Embodiment 168. The composition of any one of the above embodiments, wherein each of the MHC class I epitopes is predicted or validated to be capable of presentation by at least one HLA allele, wherein each antigen/HLA pair has an antigen/HLA prevalence of at least 0.01% in a population.

Embodiment 169. The composition of any one of the above embodiments, wherein each of the MHC class I epitopes is predicted or validated to be capable of presentation by at least one HLA allele, wherein each antigen/HLA pair has an antigen/HLA prevalence of at least 0.1% in a population.

Embodiment 170. The composition of any one of the above embodiments, wherein the CTA-associated MHC class I epitope is validated to be capable of presentation by at least one HLA allele, wherein each antigen/HLA pair has an antigen/HLA prevalence of at least 0.1% in a population.

Embodiment 171. The composition of any one of the above embodiments, wherein the CTA-associated MHC class I epitope is validated to be capable of presentation by at least one HLA allele, wherein each antigen/HLA pair has an antigen/HLA prevalence of at least 0.5% in a population.

Embodiment 172. The composition of any one of the above embodiments, wherein each of the CTA-associated MHC class I epitope is validated to be capable of presentation by at least one HLA allele, wherein each antigen/HLA pair has an antigen/HLA prevalence of at least 0.1% in a population.

Embodiment 173. The composition of any one of the above embodiments, wherein each of the CTA-associated MHC class I epitope is validated to be capable of presentation by at least one HLA allele, wherein each antigen/HLA pair has an antigen/HLA prevalence of at least 0.5% in a population.

Embodiment 174. The composition of any one of embodiments 170-173, wherein the at least one HLA allele is HLA A*01:01, HLA A*02:01, B*44:02, B*44:05, B*40:01, B*40:02, B*41:02, B*35:01, B*15:01, A*33:03, A*02:05, A*11:01, C*03:04, A*29:02, C*15:02, and/or B*07:02.

Embodiment 175. The composition of any one of the above embodiments, wherein the cassette does not encode a non-therapeutic MHC class I or class II epitope nucleic acid sequence comprising a translated, wild-type nucleic acid sequence, wherein the non-therapeutic epitope is predicted to be displayed on an MHC allele of the subject.

Embodiment 176. The composition of embodiment 175, wherein the non-therapeutic predicted MHC class I or class II epitope sequence is a junctional epitope sequence formed by adjacent sequences in the cassette.

Embodiment 177. The composition of embodiments 164-176, wherein the prediction is based on presentation likelihoods generated by inputting sequences of the non-therapeutic epitopes into a presentation model.

Embodiment 178. The composition of any one of embodiments 164-177, wherein an order of the at least one antigen-encoding nucleic acid sequences in the cassette is determined by a series of steps comprising:
(a) generating a set of candidate cassette sequences corresponding to different orders of the at least one antigen-encoding nucleic acid sequences;
(b) determining, for each candidate cassette sequence, a presentation score based on presentation of non-therapeutic epitopes in the candidate cassette sequence; and
(c) selecting a candidate cassette sequence associated with a presentation score below a predetermined threshold as the cassette sequence for an antigen vaccine.

Embodiment 179. A pharmaceutical composition comprising the composition of any one of the above embodiments and a pharmaceutically acceptable carrier.

Embodiment 180. The composition of embodiment 179, wherein the composition further comprises an adjuvant.

Embodiment 181. The pharmaceutical composition of embodiment 179 or 180, wherein the composition further comprises an immune modulator.

Embodiment 182. The pharmaceutical composition of embodiment 181, wherein the immune modulator is an anti-CTLA4 antibody or an antigen-binding fragment thereof, an anti-PD-1 antibody or an antigen-binding fragment thereof, an anti-PD-L1 antibody or an antigen-binding fragment thereof, an anti-4-1BB antibody or an antigen-binding fragment thereof, or an anti-OX-40 antibody or an antigen-binding fragment thereof.

Embodiment 183. An isolated nucleotide sequence or set of isolated nucleotide sequences comprising the cassette of any of the above composition embodiments and one or more elements obtained from the sequence of SEQ ID NO:3 or SEQ ID NO:5, optionally wherein the one or more elements are selected from the group consisting of the sequences necessary for nonstructural protein-mediated amplification, the 26S promoter nucleotide sequence, the poly(A) sequence, and the nsP1-4 genes of the sequence set forth in SEQ ID NO:3 or SEQ ID NO:5, and optionally wherein the nucleotide sequence is cDNA.

Embodiment 184. The isolated nucleotide sequence of embodiment 183, wherein the sequence or set of isolated nucleotide sequences comprises the cassette of any of the above composition embodiments inserted at position 7544 of the sequence set forth in SEQ ID NO:6 or SEQ ID NO:7.

Embodiment 185. The isolated nucleotide sequence of embodiment 183 or 184, further comprising:
a) a T7 or SP6 RNA polymerase promoter nucleotide sequence 5′ of the one or more elements obtained from the sequence of SEQ ID NO:3 or SEQ ID NO:5; and
b) optionally, one or more restriction sites 3′ of the poly(A) sequence.

Embodiment 186. The isolated nucleotide sequence of embodiment 183, wherein the cassette of any of the above composition embodiments is inserted at position 7563 of SEQ ID NO:8 or SEQ ID NO:9.

Embodiment 187. A vector or set of vectors comprising the nucleotide sequence of embodiments 183-186.

Embodiment 188. An isolated cell comprising the nucleotide sequence or set of isolated nucleotide sequences of embodiments 183-187, optionally wherein the cell is a BHK-21, CHO, HEK293 or variants thereof, 911, HeLa, A549, LP-293, PER.C6, or AE1-2a cell.

Embodiment 189. A kit comprising the composition of any of the above composition or pharmaceutical composition embodiments and instructions for use.

Embodiment 190. A method for treating a subject with cancer, the method comprising administering to the subject the composition of any of the above composition embodiments or any of the above the pharmaceutical composition embodiments.

Embodiment 191. The method of embodiment 190, wherein the at least one epitope-encoding nucleic acid sequence is derived from the tumor of the subject with cancer or from a cell or sample of the infected subject.

Embodiment 192. The method of embodiment 190, wherein the at least one epitope-encoding nucleic acid sequence are not derived from the tumor of the subject with cancer or from a cell or sample of the infected subject.

Embodiment 193. A method for stimulating an immune response in a subject, the method comprising administering to the subject the composition of any of the above composition embodiments or any of the above pharmaceutical compositions.

Embodiment 194. The method any of embodiments 190-193, wherein the subject expresses at least one HLA allele predicted or known to present the MHC class I epitope.

Embodiment 195. The method of any of embodiments 190-194, wherein the composition is administered intramuscularly (IM), intradermally (ID), subcutaneously (SC), or intravenously (IV).

Embodiment 196. The method of any of embodiments 190-194, wherein the composition is administered intramuscularly.

Embodiment 197. The method of any of embodiments 190-196, the method further comprising administration of one or more immune modulators, optionally wherein the immune modulator is administered before, concurrently with, or after administration of the composition or pharmaceutical composition.

Embodiment 198. The method of embodiment 197, wherein the one or more immune modulators are selected from the group consisting of: an anti-CTLA4 antibody or an antigen-binding fragment thereof, an anti-PD-1 antibody or an antigen-binding fragment thereof, an anti-PD-L1 antibody or an antigen-binding fragment thereof, an anti-4-1BB antibody or an antigen-binding fragment thereof, or an anti-OX-40 antibody or an antigen-binding fragment thereof.

Embodiment 199. The method of embodiment 197 or 198, wherein the immune modulator is administered intravenously (IV), intramuscularly (IM), intradermally (ID), or subcutaneously (SC).

Embodiment 200. The method of embodiment 199, wherein the subcutaneous administration is near the site of the composition or pharmaceutical composition administration or in close proximity to one or more vector or composition draining lymph nodes.

Embodiment 201. The method of any one of embodiments 190-200, further comprising administering to the subject a second vaccine composition.

Embodiment 202. The method of embodiment 201, wherein the second vaccine composition is administered prior to the administration of the composition or the pharmaceutical composition of any one of embodiments 190-202.

Embodiment 203. The method of embodiment 201, wherein the second vaccine composition is administered subsequent to the administration of the composition or the pharmaceutical composition of any one of embodiments 190-200.

Embodiment 204. The method of embodiment 202 or 203, wherein the second vaccine composition is the same as the composition or the pharmaceutical composition of any one of embodiments 190-200.

Embodiment 205. The method of embodiment 202 or 203, wherein the second vaccine composition is different from the composition or the pharmaceutical composition of any one of embodiments 190-200.

Embodiment 206. The method of embodiment 205, wherein the second vaccine composition comprises a chimpanzee adenovirus vector encoding at least one antigen-encoding nucleic acid sequence.

Embodiment 207. The method of embodiment 206, wherein the at least one antigen-encoding nucleic acid sequence encoded by the chimpanzee adenovirus vector is the same as the at least one antigen-encoding nucleic acid sequence of any of the above composition embodiments.

Embodiment 208. A method of manufacturing the one or more vectors of any of the above composition embodiments, the method comprising:
(a) obtaining a linearized DNA sequence comprising the backbone and the cassette;
(b) in vitro transcribing the linearized DNA sequence by addition of the linearized DNA sequence to an in vitro transcription reaction containing all the necessary components to transcribe the linearized DNA sequence into RNA, optionally further comprising in vitro addition of the m7 g cap to the resulting RNA; and
(c) isolating the one or more vectors from the in vitro transcription reaction.

Embodiment 209. The method of manufacturing of embodiment 208, wherein the linearized DNA sequence is generated by linearizing a DNA plasmid sequence or by amplification using PCR.

Embodiment 210. The method of manufacturing of embodiment 209, wherein the DNA plasmid sequence is generated using one of bacterial recombination or full genome DNA synthesis or full genome DNA synthesis with amplification of synthesized DNA in bacterial cells.

Embodiment 211. The method of manufacturing of embodiment 208, wherein isolating the one or more vectors from the in vitro transcription reaction involves one or more of phenol chloroform extraction, silica column based purification, or similar RNA purification methods.

Embodiment 212. A method of manufacturing the composition of any of the above composition embodiments for delivery of the antigen expression system, the method comprising:
(a) providing components for the nanoparticulate delivery vehicle;
(b) providing the antigen expression system; and
(c) contacting the components for the nanoparticulate delivery vehicle and the antigen expression system under conditions sufficient for the nanoparticulate delivery vehicle and the antigen expression system to produce the composition for delivery of the antigen expression system.

Embodiment 213. The method of manufacturing of embodiment 212, wherein the conditions are provided by microfluidic mixing.

Embodiment 214. The method of manufacturing of embodiment 212 or 213, wherein the CTA-encoding nucleic acid sequence and the KRAS-encoding nucleic acid sequence are encoded on separate vectors and mixed prior to the contacting step (c).

Embodiment 215. The method of manufacturing of embodiment 212 or 213, wherein the CTA-encoding nucleic acid sequence and the KRAS-encoding nucleic acid sequence are encoded on separate vectors in distinct antigen expression systems, and wherein the distinct antigen expression systems are independently contacted with the components for the nanoparticulate delivery vehicle.

Embodiment 216. A method for treating a subject with a disease, optionally wherein the disease is cancer or an infection, the method comprising administering to the subject one or more antigen-encoding compositions, wherein one or more antigen-encoding compositions comprise (a) a Cancer Testis Antigen (CTA)-encoding nucleic acid sequence encoding a CTA-associated MHC class I epitope and (b) a KRAS-encoding nucleic acid sequence encoding a KRAS-associated MHC class I epitope:
wherein each of the CTA-encoding nucleic acid sequence and the KRAS-encoding nucleic acid sequence is described, from 5′ to 3′, by the formula:

(Ex-(ENn)y)z
wherein,
wherein E represents a nucleotide sequence comprising a distinct epitope-encoding nucleic acid sequence,
n represents the number of separate distinct epitope-encoding nucleic acid sequences and is any integer including 0,
EN represents a nucleotide sequence comprising the separate distinct epitope-encoding nucleic acid sequence for each corresponding n,
for each iteration of z: x=0 or 1, y=0 or 1 for each n, and at least one of x or y=1, and
z=1 or greater, optionally wherein the CTA-encoding nucleic acid sequence and/or the KRAS-encoding nucleic acid sequence comprises at least two iterations of E, a given EN, or a combination thereof.

Embodiment 217. A method for treating a subject with a disease, optionally wherein the disease is cancer or an infection, the method comprising administering to the subject an antigen-based vaccine to the subject, wherein the antigen-based vaccine comprises an antigen-encoding cassette, or a polypeptide sequence encoded by the cassette, wherein the antigen-encoding cassette comprises at least one antigen-encoding nucleic acid sequence described, from 5′ to 3′, by the formula:

(Ex-(ENn)y)z
wherein,
wherein E represents a nucleotide sequence comprising a distinct epitope-encoding nucleic acid sequence,
n represents the number of separate distinct epitope-encoding nucleic acid sequences and is any integer including 0,
EN represents a nucleotide sequence comprising the separate distinct epitope-encoding nucleic acid sequence for each corresponding n,
for each iteration of z: x=0 or 1, y=0 or 1 for each n, and at least one of x or y=1, and
z=1 or greater, optionally wherein the antigen-encoding nucleic acid sequence comprises at least two iterations of E, a given EN, or a combination thereof, and
at least one of the distinct epitope-encoding nucleic acid sequences encodes a distinct shared Cancer Testis Antigen (CTA)-associated MHC class I epitope, optionally wherein at least one of the distinct epitope-encoding nucleic acid sequences encoding the CTA comprises at least two iterations.

Embodiment 218. A method for treating a subject with a disease, optionally wherein the disease is cancer, the method comprising administering to the subject an antigen-based vaccine to the subject, wherein the antigen-based vaccine comprises an antigen expression system, comprising:
the antigen expression system,
wherein the antigen expression system comprises one or more vectors,
the one or more vectors comprising (A) a Cancer Testis Antigen (CTA)-encoding nucleic acid sequence encoding a CTA-associated MHC class I epitope and (B) a KRAS-encoding nucleic acid sequence encoding a KRAS-associated MHC class I epitope,
wherein each of the one or more vectors independently comprise:
(a) a vector backbone, wherein the backbone comprises:
(i) at least one promoter nucleotide sequence, and
(ii) optionally, at least one polyadenylation (poly(A)) sequence; and
(b) a cassette, wherein the cassette comprises:
(i) at least one antigen-encoding nucleic acid sequence, comprising:
(I) at least one epitope-encoding nucleic acid sequence the CTA-associated MHC class I epitope and/or the KRAS-associated MHC class I epitope, optionally wherein at least one of the epitope-encoding nucleic acid sequences encoding the CTA-associated MHC class I epitope and/or the KRAS-associated MHC class I epitope comprises at least two iterations, and
wherein each of the epitope-encoding nucleic acid sequences comprises;
(A) optionally, a 5′ linker sequence, and
(B) optionally, a 3′ linker sequence;
(ii) optionally, a second promoter nucleotide sequence operably linked to the antigen-encoding nucleic acid sequence; and
(iii) optionally, at least one MHC class II epitope-encoding nucleic acid sequence;
(iv) optionally, at least one nucleic acid sequence encoding a GPGPG amino acid linker sequence (SEQ ID NO:56); and
(v) optionally, at least one second poly(A) sequence, wherein the second poly(A) sequence is a native poly(A) sequence or an exogenous poly(A) sequence to the vector backbone,
wherein if the second promoter nucleotide sequence is absent, the antigen-encoding nucleic acid sequence is operably linked to the at least one promoter nucleotide sequence.

Embodiment 219. A method for treating a subject with a disease, optionally wherein the disease is cancer, the method comprising administering to the subject an antigen-based vaccine to the subject, wherein the antigen-based vaccine comprises an antigen expression system, comprising: the antigen expression system,
wherein the antigen expression system comprises one or more vectors,
the one or more vectors comprising:
(a) a vector backbone, wherein the backbone comprises:
(i) at least one promoter nucleotide sequence, and
(ii) optionally, at least one polyadenylation (poly(A)) sequence; and
(b) a cassette, wherein the cassette comprises:
(i) at least one antigen-encoding nucleic acid sequence, comprising:
(I) at least one epitope-encoding nucleic acid sequence encoding a CTA-associated MHC class I epitope, and
wherein each of the epitope-encoding nucleic acid sequences comprises;
(A) optionally, a 5′ linker sequence, and
(B) optionally, a 3′ linker sequence;
(ii) optionally, a second promoter nucleotide sequence operably linked to the antigen-encoding nucleic acid sequence; and
(iii) optionally, at least one MHC class II epitope-encoding nucleic acid sequence;
(iv) optionally, at least one nucleic acid sequence encoding a GPGPG amino acid linker sequence (SEQ ID NO:56); and
(v) optionally, at least one second poly(A) sequence, wherein the second poly(A) sequence is a native poly(A) sequence or an exogenous poly(A) sequence to the vector backbone,
wherein if the second promoter nucleotide sequence is absent, the antigen-encoding nucleic acid sequence is operably linked to the at least one promoter nucleotide sequence, and
wherein the at least one antigen-encoding nucleic acid sequence comprises at least two iterations of the epitope-encoding nucleic acid sequence encoding the CTA-associated MHC class I epitope.

Embodiment 220. The method of any one of embodiments 216-219, wherein the at least one epitope-encoding nucleic acid sequence is derived from a tumor of the subject with cancer.

Embodiment 221. The method any one of embodiments 216-219, wherein the at least one epitope-encoding nucleic acid sequence are not derived from a tumor of the subject with cancer.

Embodiment 222. A method for stimulating an immune response in a subject, the method comprising the method comprising administering to the subject one or more antigen-encoding compositions, wherein one or more antigen-encoding compositions comprise (a) a Cancer Testis Antigen (CTA)-encoding nucleic acid sequence encoding a CTA-associated MHC class I epitope and (b) a KRAS-encoding nucleic acid sequence encoding a KRAS-associated MHC class I epitope: wherein each of the CTA-encoding nucleic acid sequence and the KRAS-encoding nucleic acid sequence is described, from 5′ to 3′, by the formula:

(Ex-(ENn)y)z
wherein,
wherein E represents a nucleotide sequence comprising a distinct epitope-encoding nucleic acid sequence,
n represents the number of separate distinct epitope-encoding nucleic acid sequences and is any integer including 0,
EN represents a nucleotide sequence comprising the separate distinct epitope-encoding nucleic acid sequence for each corresponding n,
for each iteration of z: x=0 or 1, y=0 or 1 for each n, and at least one of x or y=1, and
z=1 or greater, optionally wherein the CTA-encoding nucleic acid sequence and/or the KRAS-encoding nucleic acid sequence comprises at least two iterations of E, a given EN, or a combination thereof.

Embodiment 223. A method for stimulating an immune response in a subject, the method comprising the method comprising administering to the subject an antigen-based vaccine to the subject, wherein the antigen-based vaccine comprises an antigen-encoding cassette, or a polypeptide sequence encoded by the cassette, wherein the antigen-encoding cassette comprises at least one antigen-encoding nucleic acid sequence described, from 5′ to 3′, by the formula:

(Ex-(ENn)y)z
wherein E represents a nucleotide sequence comprising a distinct epitope-encoding nucleic acid sequence,
n represents the number of separate distinct epitope-encoding nucleic acid sequences and is any integer including 0,
EN represents a nucleotide sequence comprising the separate distinct epitope-encoding nucleic acid sequence for each corresponding n,
for each iteration of z: x=0 or 1, y=0 or 1 for each n, and at least one of x or y=1, and
z=1 or greater, optionally wherein the antigen-encoding nucleic acid sequence comprises at least two iterations of E, a given EN, or a combination thereof, and
at least one of the distinct epitope-encoding nucleic acid sequences encodes a distinct shared Cancer Testis Antigen (CTA)-associated MHC class I epitope, optionally wherein at least one of the distinct epitope-encoding nucleic acid sequences encoding the CTA comprises at least two iterations.

Embodiment 224. A method for stimulating an immune response in a subject, the method comprising the method comprising administering to the subject an antigen-based vaccine to the subject, wherein the antigen-based vaccine comprises an antigen expression system, comprising: the antigen expression system,
wherein the antigen expression system comprises one or more vectors,
the one or more vectors comprising (A) a Cancer Testis Antigen (CTA)-encoding nucleic acid sequence encoding a CTA-associated MHC class I epitope and (B) a KRAS-encoding nucleic acid sequence encoding a KRAS-associated MHC class I epitope,
wherein each of the one or more vectors independently comprise:
(a) a vector backbone, wherein the backbone comprises:
(i) at least one promoter nucleotide sequence, and
(ii) optionally, at least one polyadenylation (poly(A)) sequence; and
(b) a cassette, wherein the cassette comprises:
(i) at least one antigen-encoding nucleic acid sequence, comprising:
(I) at least one epitope-encoding nucleic acid sequence the CTA-associated MHC class I epitope and/or the KRAS-associated MHC class I epitope, optionally wherein at least one of the epitope-encoding nucleic acid sequences encoding the CTA-associated MHC class I epitope and/or the KRAS-associated MHC class I epitope comprises at least two iterations, and
wherein each of the epitope-encoding nucleic acid sequences comprises;
(A) optionally, a 5′ linker sequence, and
(B) optionally, a 3′ linker sequence;
(ii) optionally, a second promoter nucleotide sequence operably linked to the antigen-encoding nucleic acid sequence; and
(iii) optionally, at least one MHC class II epitope-encoding nucleic acid sequence;
(iv) optionally, at least one nucleic acid sequence encoding a GPGPG amino acid linker sequence (SEQ ID NO:56); and
(v) optionally, at least one second poly(A) sequence, wherein the second poly(A) sequence is a native poly(A) sequence or an exogenous poly(A) sequence to the vector backbone,
wherein if the second promoter nucleotide sequence is absent, the antigen-encoding nucleic acid sequence is operably linked to the at least one promoter nucleotide sequence.

Embodiment 225. A method for stimulating an immune response in a subject, the method comprising the method comprising administering to the subject an antigen-based vaccine to the subject, wherein the antigen-based vaccine comprises:
an antigen expression system, comprising:
the antigen expression system,
wherein the antigen expression system comprises one or more vectors,
the one or more vectors comprising:
(a) a vector backbone, wherein the backbone comprises:
(i) at least one promoter nucleotide sequence, and
(ii) optionally, at least one polyadenylation (poly(A)) sequence; and
(b) a cassette, wherein the cassette comprises:
(i) at least one antigen-encoding nucleic acid sequence, comprising:
(I) at least one epitope-encoding nucleic acid sequence encoding a CTA-associated MHC class I epitope, and
wherein each of the epitope-encoding nucleic acid sequences comprises;
(A) optionally, a 5′ linker sequence, and
(B) optionally, a 3′ linker sequence;
(ii) optionally, a second promoter nucleotide sequence operably linked to the antigen-encoding nucleic acid sequence; and
(iii) optionally, at least one MHC class II epitope-encoding nucleic acid sequence;
(iv) optionally, at least one nucleic acid sequence encoding a GPGPG amino acid linker sequence (SEQ ID NO:56); and
(v) optionally, at least one second poly(A) sequence, wherein the second poly(A) sequence is a native poly(A) sequence or an exogenous poly(A) sequence to the vector backbone,
wherein if the second promoter nucleotide sequence is absent, the antigen-encoding nucleic acid sequence is operably linked to the at least one promoter nucleotide sequence, and
wherein the at least one antigen-encoding nucleic acid sequence comprises at least two iterations of the epitope-encoding nucleic acid sequence encoding the CTA-associated MHC class I epitope.

Embodiment 226. The method of any one of embodiments 216-225, wherein the subject expresses at least one HLA allele predicted or known to present the CTA-associated MHC class I epitope and/or the KRAS-associated MHC class I epitope.

Embodiment 227. The method of any one of embodiments 216-225, wherein the subject expresses at least one HLA allele predicted or known to present the at least one epitope sequence, and wherein the at least one epitope sequence comprises an epitope known or suspected to be presented by MHC class I on a surface of a cell, wherein the at least one epitope sequence predicted or known to be presented comprises the CTA-associated MHC class I epitope and/or the KRAS-associated MHC class I epitope.

Embodiment 228. The method of embodiment 227, wherein the surface of the cell is a tumor cell surface.

Embodiment 229. The method of embodiment 227, wherein the cell is a tumor cell selected from the group consisting of: lung cancer, melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, T cell lymphocytic leukemia, non-small cell lung cancer, and small cell lung cancer.

Embodiment 230. The method of embodiment 227, wherein the cell is a lung adenocarcinoma, ovarian serous cystadenocarcinoma, lung squamous cell carcinoma, colon cancer, or head and neck squamous cell carcinoma tumor cell.

Embodiment 231. A method for inducing an immune response in a subject, the method comprising the method comprising administering to the subject one or more antigen-encoding compositions, wherein one or more antigen-encoding compositions comprise (a) a Cancer Testis Antigen (CTA)-encoding nucleic acid sequence encoding a CTA-associated MHC class I epitope and (b) a KRAS-encoding nucleic acid sequence encoding a KRAS-associated MHC class I epitope:
wherein each of the CTA-encoding nucleic acid sequence and the KRAS-encoding nucleic acid sequence is described, from 5′ to 3′, by the formula:

(Ex-(ENn)y)z
wherein,
wherein E represents a nucleotide sequence comprising a distinct epitope-encoding nucleic acid sequence,
n represents the number of separate distinct epitope-encoding nucleic acid sequences and is any integer including 0,
EN represents a nucleotide sequence comprising the separate distinct epitope-encoding nucleic acid sequence for each corresponding n,
for each iteration of z: x=0 or 1, y=0 or 1 for each n, and at least one of x or y=1, and
z=1 or greater, optionally wherein the CTA-encoding nucleic acid sequence and/or the KRAS-encoding nucleic acid sequence comprises at least two iterations of E, a given EN, or a combination thereof.

Embodiment 232. A method for inducing an immune response in a subject, the method comprising administering to the subject an antigen-based vaccine to the subject, wherein the antigen-based vaccine comprises an antigen-encoding cassette, or a polypeptide sequence encoded by the cassette, wherein the antigen-encoding cassette comprises at least one antigen-encoding nucleic acid sequence described, from 5′ to 3′, by the formula:

(Ex-(ENn)y)z
wherein E represents a nucleotide sequence comprising a distinct epitope-encoding nucleic acid sequence,
n represents the number of separate distinct epitope-encoding nucleic acid sequences and is any integer including 0,
EN represents a nucleotide sequence comprising the separate distinct epitope-encoding nucleic acid sequence for each corresponding n,
for each iteration of z: x=0 or 1, y=0 or 1 for each n, and at least one of x or y=1, and
z=1 or greater, optionally wherein the antigen-encoding nucleic acid sequence comprises at least two iterations of E, a given EN, or a combination thereof, and
at least one of the distinct epitope-encoding nucleic acid sequences encodes a distinct shared Cancer Testis Antigen (CTA)-associated MHC class I epitope, optionally wherein at least one of the distinct epitope-encoding nucleic acid sequences encoding the CTA comprises at least two iterations.

Embodiment 233. A method for inducing an immune response in a subject, the method comprising administering to the subject an antigen-based vaccine to the subject, wherein the antigen-based vaccine comprises:
an antigen expression system, comprising:
the antigen expression system,
wherein the antigen expression system comprises one or more vectors,
the one or more vectors comprising (A) a Cancer Testis Antigen (CTA)-encoding nucleic acid sequence encoding a CTA-associated MHC class I epitope and (B) a KRAS-encoding nucleic acid sequence encoding a KRAS-associated MHC class I epitope,
wherein each of the one or more vectors independently comprise:
(a) a vector backbone, wherein the backbone comprises:
(i) at least one promoter nucleotide sequence, and
(ii) optionally, at least one polyadenylation (poly(A)) sequence; and
(b) a cassette, wherein the cassette comprises:
(i) at least one antigen-encoding nucleic acid sequence, comprising:
(I) at least one epitope-encoding nucleic acid sequence the CTA-associated MHC class I epitope and/or the KRAS-associated MHC class I epitope, optionally wherein at least one of the epitope-encoding nucleic acid sequences encoding the CTA-associated MHC class I epitope and/or the KRAS-associated MHC class I epitope comprises at least two iterations, and
wherein each of the epitope-encoding nucleic acid sequences comprises;
(A) optionally, a 5′ linker sequence, and
(B) optionally, a 3′ linker sequence;
(ii) optionally, a second promoter nucleotide sequence operably linked to the antigen-encoding nucleic acid sequence; and
(iii) optionally, at least one MHC class II epitope-encoding nucleic acid sequence;
(iv) optionally, at least one nucleic acid sequence encoding a GPGPG amino acid linker sequence (SEQ ID NO:56); and
(v) optionally, at least one second poly(A) sequence, wherein the second poly(A) sequence is a native poly(A) sequence or an exogenous poly(A) sequence to the vector backbone,
wherein if the second promoter nucleotide sequence is absent, the antigen-encoding nucleic acid sequence is operably linked to the at least one promoter nucleotide sequence, and
wherein the subject expresses at least one HLA allele predicted or known to present the CTA-associated MHC class I epitope and/or the KRAS-associated MHC class I epitope.

Embodiment 234. A method for inducing an immune response in a subject, the method comprising administering to the subject an antigen-based vaccine to the subject, wherein the antigen-based vaccine comprises:
an antigen expression system, comprising:
the antigen expression system,
wherein the antigen expression system comprises one or more vectors,
the one or more vectors comprising:
(a) a vector backbone, wherein the backbone comprises:
(i) at least one promoter nucleotide sequence, and
(ii) optionally, at least one polyadenylation (poly(A)) sequence; and
(b) a cassette, wherein the cassette comprises:
(i) at least one antigen-encoding nucleic acid sequence, comprising:
(I) at least one epitope-encoding nucleic acid sequence encodes a CTA-associated MHC class I epitope, optionally wherein at least one of the epitope-encoding nucleic acid sequences encoding the CTA comprises at least two iterations, and
wherein each of the epitope-encoding nucleic acid sequences comprises;
(A) optionally, a 5′ linker sequence, and
(B) optionally, a 3′ linker sequence;
(ii) optionally, a second promoter nucleotide sequence operably linked to the antigen-encoding nucleic acid sequence; and
(iii) optionally, at least one MHC class II epitope-encoding nucleic acid sequence;
(iv) optionally, at least one nucleic acid sequence encoding a GPGPG amino acid linker sequence (SEQ ID NO:56); and
(v) optionally, at least one second poly(A) sequence, wherein the second poly(A) sequence is a native poly(A) sequence or an exogenous poly(A) sequence to the vector backbone,
wherein if the second promoter nucleotide sequence is absent, the antigen-encoding nucleic acid sequence is operably linked to the at least one promoter nucleotide sequence, and
wherein the subject expresses at least one HLA allele predicted or known to present the at least one CTA-associated MHC class I epitope.

Embodiment 235. The method of any one of embodiments 216-234, wherein the CTA-encoding nucleic acid sequence and the KRAS-encoding nucleic acid sequence are encoded in the same cassette and/or vector.

Embodiment 236. The method of embodiment 235, wherein the CTA-encoding nucleic acid sequence and the KRAS-encoding nucleic acid sequence are linked together by a 2A ribosome skipping sequence.

Embodiment 237. The method of embodiment 235, wherein the CTA-encoding nucleic acid sequence and the KRAS-encoding nucleic acid sequence are each independently operably linked to a separate promoter.

Embodiment 238. The method of embodiment 216-234, wherein the CTA-encoding nucleic acid sequence and the KRAS-encoding nucleic acid sequence are encoded on separate vectors.

Embodiment 239. The method of embodiment 238, wherein the CTA-encoding nucleic acid sequence and the KRAS-encoding nucleic acid sequence are co-administered.

Embodiment 240. The method of embodiment 238 or 239, wherein the CTA-encoding nucleic acid sequence and the KRAS-encoding nucleic acid sequence are co-formulated.

Embodiment 241. The method of embodiment 238 or 239, wherein the CTA-encoding nucleic acid sequence and the KRAS-encoding nucleic acid sequence are administered at separate injection sites.

Embodiment 242. The method of embodiment 241, wherein the administration at separate injection sites comprises bilateral administration.

Embodiment 243. The method of any of embodiments 216-242, wherein the antigen expression system comprises any one of the antigen expression systems in any one of embodiments 1-178.

Embodiment 244. The method of any of embodiments 216-242, wherein the antigen-based vaccine comprises any one of the pharmaceutical compositions in any one of embodiments 179-192.

Embodiment 245. The method of any of embodiments 216-244, wherein the antigen-based vaccine is administered as a priming dose.

Embodiment 246. The method of any of embodiments 216-245, wherein the antigen-based vaccine is administered as one or more boosting doses.

Embodiment 247. The method of embodiment 246, wherein the boosting dose is different than the priming dose.

Embodiment 248. The method of embodiment 247, wherein:
a) the priming dose comprises a chimpanzee adenovirus vector and the boosting dose comprises an alphavirus vector; or
b) the priming dose comprises an alphavirus vector and the boosting dose comprises a chimpanzee adenovirus vector.

Embodiment 249. The method of embodiment 246, wherein the boosting dose is the same as the priming dose.

Embodiment 250. The method of any one of embodiments 246-249, wherein the injection site of the one or more boosting doses is as close as possible to the injection site of the priming dose.

Embodiment 251. The method of any one of the above method embodiments, further comprising determining or having determined the HLA-haplotype of the subject.

Embodiment 252. The method of any one of the above method embodiments, wherein the antigen-based vaccine is administered intramuscularly (IM), intradermally (ID), subcutaneously (SC), or intravenously (IV).

Embodiment 253. The method of any one of the above method embodiments, wherein the antigen-based vaccine is administered intramuscularly (IM).

Embodiment 254. The method of embodiment 253, wherein the IM administration is administered at separate injection sites.

Embodiment 255. The method of embodiment 254, wherein the separate injection sites are in opposing deltoid muscles.

Embodiment 256. The method of embodiment 255, wherein the separate injection sites are in gluteus or rectus femoris sites on each side.

Embodiment 257. The method or composition of any of the above embodiments, wherein the CTA-encoding nucleic acid sequence encodes the amino acid sequence

ELGVMGVYDGREHTVYGEPRKELGVMGVYDGREHTVYGEPRKGID
VKEVDPTSHSYVLVTSGPRALAETSYVKVLEHVVRVNARVRGPRA
LAETSYVKVLEYVIKVSARVRFFFFLNMLGVYDGEEHSVFGEPWF
QRNTGEMSSNSTALALVRPSSSGLINSNTDNNLAVYDLSRDSPQG
ASALPTTISFTCWRQGIELMEVDPIGHLYIFATCFQRNTGEMSSN
STALALVRPSSSGLINSNTDNNLAVYDLSRDSPQGASALPTTISF
TCWRQGIELMEVDPIGHLYIFATCGPRALAETSYVKVLEHVVRVN
ARVRGPRALAETSYVKVLEYVIKVSARVRFFFFLNMLGVYDGEEH
SVFGEPWGIDVKEVDPTSHSYVLVTS.

Embodiment 258. The method or composition of any of the above embodiments, wherein the CTA-encoding nucleic acid sequence encodes the amino acid sequence

GPRALAETSYVKVLEHVVRVNARVRIAYPSLREAALLEEEEGVWL
EEGPRQSLQQCVAISLLTQYFVQENYLEYRQVPGMVENKLVELEH
TLLSKGIELMEVDPIGHLYIFATCQRNTGEMSSNSTALALVRPSS
SGLINSNTDNNLAVYDLSRWEELGVMGVYDGREHTVYGEPRKLLT
QDQRNTGEMSSNSTALALVRPSSSGLINSNTDNNLAVYDLSRWEE
LGVMGVYDGREHTVYGEPRKLLTQDWMVENKLVELEHTLLSKGIE
LMEVDPIGHLYIFATCPRALAETSYVKVLEHVVRVNARVRIAYPS
LREAALLEEEEGVWLEEGPRQSLQQCVAISLLTQYFVQENYLEYR
QVPG.

Embodiment 259. The method or composition of any of the above embodiments, wherein the at least one promoter sequence is a CMV, SV40, EF-1, RSV, PGK, HSA, MCK or EBV promoter sequence.

Embodiment 260. The method or composition of any of the above embodiments, wherein the at least one promoter sequence is a regulatable promoter, optionally wherein the regulatable promoter is a tetracycline (TET) repressor protein (TETr) controlled promoter, optionally wherein the regulatable promoter comprises multiple TET operator (TETo) sequences 5′ or 3′ of a RNA polymerase binding sequence of the promoter.

Embodiment 261. The method or composition of any of the above embodiments, wherein P comprises a CMV-derived promoter sequence, optionally wherein the CMV-derived promoter sequence comprises a TETr controlled CMV-derived promoter.

### XIV. Examples

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B(1992).

XIV.A. Evaluation of Cassettes with Shared CTA Epitopes

Through vaccination, multiple class I MHC restricted antigens that stimulate the corresponding cellular immune response(s) can be delivered. Several vaccine cassettes were engineered to encode multiple mutant KRAS epitopes (e.g., G12C, G12V, G12D, and/or Q61H) and multiple shared CTA epitopes, as a single gene product where the epitopes were embedded within their natural, surrounding peptide sequence. Various cassettes designs also feature multiple iterations (copies) of the shared CTA epitopes and/or the KRAS epitopes. Several vaccine cassettes were engineered to encode multiple shared CTA epitopes, as a single gene product where the epitopes are embedded within their natural, surrounding peptide sequence.

### XVI.B. CTA Epitope Vaccine Evaluation Materials and Methods

### Antigens

The shared CTA epitopes used in the examples below are presented in Table 2A. The antigen-encoding sequences of the cassettes for the various constructs were constructed by directly linking (iterating) sequences ranging from 19 to 42 residues in length (i.e., an epitope flanked by a minimum of 5 native N and C terminal amino acid linkers) to each other (i.e., no additional amino acids in between consecutive sequences) in the order and number described in the examples below. The cassettes containing the standard and full-length antigen-encoding sequences containing the iterated CTA epitopes linked together are presented in Table 2B. The linked sequences of the cassettes in the standard-length antigen cassette range from 19 to 42 residues in length (i.e., an epitope flanked by a minimum of 5 native N and C terminal amino acid linkers) to each other (i.e., no additional amino acids in between consecutive sequences). The linked sequences in the full-length antigen cassette range from 25 to 42 residues in length (i.e., an epitope flanked by a native N and C terminal amino acid linkers to generate minimum linked sequence lengths of 25 residues and up to 42 residues). Cassettes containing antigen-encoding sequences containing the iterated CTA epitopes are also encoded together with antigen-encoding sequences containing KRAS G12V and G12C neoepitopes iterated in a “2×4” format or together with antigen-encoding sequences containing KRAS G12V, G12C, G12D, and Q61H neoepitopes iterated in a “4×4” format presented in Table 2B, including constructs with CTA and KRAS antigen-encoding sequences linked together with a T2A ribosome skipping sequence element or a second promoter (an additional 26S subgenomic promoter sequence for samRNA constructs or a EF1alpha promoter for ChAdV constructs). The complete exogenous nucleotide insertions into the vectors include from 5′ to 3′: a Kozak sequence (GCCACC), nucleotides encoding three amino acids MAG (ATGGCCGGG), one of the cassette sequences of Table 2B, nucleotides encoding a universal MHC class II antigens tetanus toxoid and PADRE with a N-terminal GPGPG linker, and two stop codons (TAATGA). Additional constructs are assessed with various different dual promoter combinations, such as using different orders of CMV and EF1alpha promoters, or substituting the CMV and/or EF1alpha promoter for a different promoter.

TABLE 2A
CTA Epitope And Universal MHC-II Sequences

| Gene | Allele | Sequence | SEQ ID NO: |
|---|---|---|---|
| MAGEA4/A1/A8 | A*11:01 | ALAETSYVK | |
| MAGEA4 | B*07:02 | YPSLREAAL | |
| MAGEA4 | C*03:04 | IAYPSLREAAL | |
| MAGEA3/A6 | A*01:01 | FVQENYLEY | |
| | A*29:02 | | |
| MAGEA11 | A*01:01 | EVDPTSHSY | |
| CT83 | A*01:01 | NTDNNLAVY | |
| MAGEA3 | A*01:01 | EVDPIGHLY | |
| MAGEA3 | B*40:01 | MEVDPIGHL | |
| MAGEA4 | A*02:01 | GVYDGREHTV | |
| | A*02:05 | | |
| MAGEA4 | A*02:01 | ALLEEEEGV | |
| MAGEA4/A8 | A*02:01 | KVLEHVVRV | |
| | C*15:02 | | |
| MAGEA1 | A*02:01 | KVLEYVIKV | |
| MAGEB2 | A*02:01 | GVYDGEEHSV | |
| CT83 | A*02:01 | KLVELEHTL | |
| MAGEA1/A4 | B*44:02 | AETSYVKVL | |
| | B*44:05 | | |
| | B*40:01 | | |
| | B*40:02 | | |
| | B*41:02 | | |
| MAGEA1 | A*33:03 | EYVIKVSAR | |
| MAGEA4 | B*35:01 | SALPTTISF | |
| MAGEA4 | B*15:01 | GVYDGREHTVY | |
| CTCFL | B*07:02 | GPRQSLQQC | |
| CT83 | A*01:01 | NTDNNLAVY | |
| CT83 | B*07:02 | LVRPSSSGL | |
| CT83 | B*40:01 | GEMSSNSTAL | |
| Tetanus toxoid and | N/A | GPGPGAKFVA | |
| PADRE with GPGPG | | AWTLKAAAGP | |
| linkers (bold) | | GPGQYIKANS | |
| | | KFIGITELGP | |
| | | GPG | |

TABLE 2B
Full-Length CTA and KRAS Cassettes

CTA 1x Iteration Cassette AA (Standard)
ELGVMGVYDGREHTVYGEPRKGIDVKEVDPTSHSYVLVTSGIELMEVDPIGHLYIFATCF
QRNTGEMSSNSTALALVRPSSSGLINSNTDNNLAVYDLSRDSPQGASALPTTISFTCWRQ
GPRALAETSYVKVLEHVVRGPRALAETSYVKVLEYVIKVSARVRFFFFLNMLGVYDGEEH
SVFGEPW
CTA 8x1 (Single interation) V2 Cassette AA
ELGVMGVYDGREHTVYGEPRKGIDVKEVDPTSHSYVLVTSGIELMEVDPIGHLYIFATCF
QRNTGEMSSNSTALALVRPSSSGLINSNTDNNLAVYDLSRDSPQGASALPTTISFTCWRQ
GPRALAETSYVKVLEHVVRVNARVRGPRALAETSYVKVLEYVIKVSARVRFFFFLNMLGV
YDGEEHSVFGEPW
CTA8x1 (Single interation) V2 Cassette NT
GAATTGGGAGTAATGGGcGTTTATGACGGCCGTGAGCACACTGTTTATGGCGAACCAAGG
AAGGGAATAGACGTGAAGGAAGTGGACCCTACCAGTCATAGTTATGTGCTCGTCACAAGT
GGGATCGAGCTAATGGAGGTTGATCCCATTGGGCATTTGTACATCTTCGCGACCTGCTTT
CAACGAAATACGGGTGAAATGAGCTCTAATAGTACTGCATTAGCTCTTGTTCGTCCATCT
TCAAGCGGCCTCATTAATTCTAATACCGACAACAACCTCGCAGTGTATGACCTATCACGA
GACAGTCCACAGGGAGCGTCCGCCCTGCCAACTACCATCAGCTTCACGTGTTGGAGACAG
GGCCCCAGAGCACTGGCTGAGACATCATATGTGAAAGTGCTCGAACACGTTGTGAGGGTG
AATGCTAGAGTGCGTGGACCCCGCGCCCTCGCTGAAACTAGTTACGTCAAGGTGCTGGAG
TACGTGATAAAAGTCTCAGCCAGGGTTAGATTTTTCTTCTTTCTGAACATGCTGGGAGTC
TATGATGGTGAGGAGCACAGCGTCTTTGGTGAGCCATGG
CTA 1x Iteration Cassette NT (Standard)
GAATTGGGAGTAATGGGAGTTTATGACGGCCGTGAGCACACTGTTTATGGCGAACCAAGG
AAGGGAATAGACGTGAAGGAAGTGGACCCTACCAGTCATAGTTATGTGCTCGTCACAAGT
GGGATCGAGCTAATGGAGGTTGATCCCATTGGGCATTTGTACATCTTCGCGACCTGCTTT
CAACGAAATACGGGTGAAATGAGCTCTAATAGTACTGCATTAGCTCTTGTTCGTCCATCT
TCAAGCGGCCTCATTAATTCTAATACCGACAACAACCTCGCAGTGTATGACCTATCACGA
GACAGTCCACAGGGAGCGTCCGCCCTGCCAACTACCATCAGCTTCACGTGTTGGAGACAG
GGCCCCAGAGCACTGGCTGAGACATCATATGTGAAAGTGCTCGAACACGTTGTGAGGGGA
CCCCGCGCCCTCGCTGAAACTAGTTACGTCAAGGTGCTGGAGTACGTGATAAAAGTCTCA
GCCAGGGTTAGATTTTTCTTCTTTCTGAACATGCTGGGAGTCTATGATGGTGAGGAGCAC
AGCGTCTTTGGTGAGCCATGG
CTA 2x Iteration Cassette AA (Standard)
ELGVMGVYDGREHTVYGEPRKELGVMGVYDGREHTVYGEPRKGIDVKEVDPTSHSYVLVT
SGPRALAETSYVKVLEHVVRGPRALAETSYVKVLEYVIKVSARVRFFFFLNMLGVYDGEE
HSVFGEPWFQRNTGEMSSNSTALALVRPSSSGLINSNTDNNLAVYDLSRDSPQGASALPT
TISFTCWRQGIELMEVDPIGHLYIFATCFQRNTGEMSSNSTALALVRPSSSGLINSNTDN
NLAVYDLSRDSPQGASALPTTISFTCWRQGIELMEVDPIGHLYIFATCGPRALAETSYVK
VLEHVVRGPRALAETSYVKVLEYVIKVSARVRFFFFLNMLGVYDGEEHSVFGEPWGIDVK
EVDPTSHSYVLVTS
CTA 2x Iteration Cassette NT (Standard)
GAACTTGGTGTCATGGGTGTCTACGATGGGAGAGAACACACGGTATACGGCGAGCCACGA
AAGGAACTCGGCGTTATGGGCGTGTATGACGGACGAGAGCACACAGTCTACGGAGAGCCG
CGCAAAGGAATAGACGTCAAGGAGGTAGACCCGACGTCACACTCTTACGTGCTCGTTACC
TCAGGTCCCCGAGCCTTGGCGGAGACATCGTATGTCAAAGTCTTGGAGCACGTTGTTCGT
GGGCCACGAGCGCTCGCGGAAACCAGCTATGTAAAGGTGTTGGAATACGTCATCAAAGTT
TCCGCGAGGGTTCGATTTTTCTTCTTCCTCAATATGCTCGGTGTATACGACGGGGAAGAG
CACTCTGTATTCGGGGAGCCATGGTTTCAACGGAACACTGGAGAGATGTCCTCCAATTCT
ACAGCTTTGGCTCTGGTGCGACCCAGTTCCTCAGGACTCATCAATTCCAACACGGATAAT
AATTTGGCAGTTTACGATCTCAGCAGGGACTCTCCACAGGGAGCGTCAGCGCTCCCCACG
ACTATCAGTTTTACCTGCTGGCGACAAGGCATCGAACTCATGGAAGTGGACCCGATTGGT
CATCTTTATATATTCGCGACTTGCTTTCAGAGGAATACCGGGGAAATGTCTTCCAACTCC
ACGGCGCTGGCATTGGTCCGGCCCAGCAGCTCCGGTCTTATCAACTCAAACACAGACAAT
AACTTGGCCGTATATGATCTATCTCGGGACAGCCCACAAGGAGCTTCTGCACTGCCGACC
ACTATTAGCTTCACCTGTTGGCGGCAGGGAATCGAGTTGATGGAGGTTGATCCTATAGGC
CACTTATACATTTTTGCCACATGCGGACCAAGAGCGCTTGCCGAAACATCCTATGTGAAG
GTGCTCGAACATGTTGTACGAGGGCCCCGGGCCCTCGCAGAAACTTCCTACGTGAAAGTC
CTGGAGTACGTTATCAAGGTGTCAGCACGAGTACGCTTCTTTTTCTTTCTAAACATGCTG
GGAGTTTATGATGGGGAGGAACATTCGGTTTTCGGCGAACCGTGGGGGATCGATGTAAAA
GAGGTGGATCCGACTTCCCACTCGTACGTCTTGGTTACGTCG
CTA 8x2 Cassette AA “Option 1”
ELGVMGVYDGREHTVYGEPRKELGVMGVYDGREHTVYGEPRKGIDVKEVDPTSHSYVLVT
SGPRALAETSYVKVLEHVVRVNARVRGPRALAETSYVKVLEYVIKVSARVRFFFFLNMLG
VYDGEEHSVFGEPWFQRNTGEMSSNSTALALVRPSSSGLINSNTDNNLAVYDLSRDSPQG
ASALPTTISFTCWRQGIELMEVDPIGHLYIFATCFQRNTGEMSSNSTALALVRPSSSGLI
NSNTDNNLAVYDLSRDSPQGASALPTTISFTCWRQGIELMEVDPIGHLYIFATCGPRALA
ETSYVKVLEHVVRVNARVRGPRALAETSYVKVLEYVIKVSARVRFFFFLNMLGVYDGEEH
SVFGEPWGIDVKEVDPTSHSYVLVTS
CTA 8x2 Cassette NT “Option 1”
GAACTTGGTGTCATGGGTGTCTACGATGGGAGAGAACACACGGTATACGGCGAGCCACGA
AAGGAACTCGGCGTTATGGGCGTGTATGACGGACGAGAGCACACAGTCTACGGAGAGCCG
CGCAAAGGAATAGACGTCAAGGAGGTAGACCCGACGTCACACTCTTACGTGCTCGTTACC
TCAGGTCCCCGAGCCTTGGCGGAGACATCGTATGTCAAAGTCTTGGAGCACGTTGTTCGT
GTGAACGCTCGGGTTAGAGGGCCACGAGCGCTCGCGGAAACCAGCTATGTAAAGGTGTTG
GAATACGTCATCAAAGTTTCCGCGAGGGTTCGATTTTTCTTCTTCCTCAATATGCTCGGT
GTATACGACGGGGAAGAGCACTCTGTATTCGGGGAGCCATGGTTTCAACGGAACACTGGA
GAGATGTCCTCCAATTCTACAGCTTTGGCTCTGGTGCGACCCAGTTCCTCAGGACTCATC
AATTCCAACACGGATAATAATTTGGCAGTTTACGATCTCAGCAGGGACTCTCCACAGGGA
GCGTCAGCGCTCCCCACGACTATCAGTTTTACCTGCTGGCGACAAGGCATCGAACTCATG
GAAGTGGACCCGATTGGTCATCTTTATATATTCGCGACTTGCTTTCAGAGGAATACCGGG
GAAATGTCTTCCAACTCCACGGCGCTGGCATTGGTCCGGCCCAGCAGCTCCGGTCTTATC
AACTCAAACACAGACAATAACTTGGCCGTATATGATCTATCTCGGGACAGCCCACAAGGA
GCTTCTGCACTGCCGACCACTATTAGCTTCACCTGTTGGCGGCAGGGAATCGAGTTGATG
GAGGTTGATCCTATAGGCCACTTATACATTTTTGCCACATGCGGACCAAGAGCGCTTGCC
GAAACATCCTATGTGAAGGTGCTCGAACATGTTGTACGAGTCAATGCTAGAGTCAGGGGG
CCCCGGGCCCTCGCAGAAACTTCCTACGTGAAAGTCCTGGAGTACGTTATCAAGGTGTCA
GCACGAGTACGCTTCTTTTTCTTTCTAAACATGCTGGGAGTTTATGATGGGGAGGAACAT
TCGGTTTTCGGCGAACCGTGGGGGATCGATGTAAAAGAGGTGGATCCGACTTCCCACTCG
TACGTCTTGGTTACGTCG
CTA 8x2 Cassette AA “Option 4”
GPRALAETSYVKVLEHVVRVNARVRIAYPSLREAALLEEEEGVWLEEGPRQSLQQCVAIS
LLTQYFVQENYLEYRQVPGMVENKLVELEHTLLSKGIELMEVDPIGHLYIFATCQRNTGE
MSSNSTALALVRPSSSGLINSNTDNNLAVYDLSRWEELGVMGVYDGREHTVYGEPRELLT
QDQRNTGEMSSNSTALALVRPSSSGLINSNTDNNLAVYDLSRWEELGVMGVYDGREHTVY
GEPRKLLTQDWMVENKLVELEHTLLSKGIELMEVDPIGHLYIFATCPRALAETSYVKVLE
HVVRVNARVRIAYPSLREAALLEEEEGVWLEEGPRQSLQQCVAISLLTQYFVQENYLEYR
QVPG
CTA 8x2 Cassette NT “Option 4”
GGACCTAGGGCCTTAGCTGAAACTAGTTATGTGAAAGTCCTCGAGCATGTAGTGAGAGTG
AACGCTAGGGTTAGGATAGCCTACCCATCCCTGAGAGAAGCTGCCCTTCTTGAGGAAGAA
GAGGGGGTGTGGCTAGAAGAAGGACCCCGGCAGAGCCTGCAGCAGTGCGTAGCCATAAGT
CTGCTGACCCAATATTTCGTCCAGGAAAATTATCTAGAGTATAGACAGGTGCCTGGAATG
GTGGAGAATAAGCTGGTGGAACTAGAACATACCTTGCTGAGTAAAGGCATTGAATTAATG
GAGGTTGACCCTATTGGTCATCTGTACATATTTGCCACCTGTCAGAGAAATACCGGTGAA
ATGAGTAGTAACAGTACGGCGCTGGCCTTGGTCAGACCCTCCAGCTCCGGGCTGATTAAT
AGCAACACAGACAATAATCTGGCCGTCTACGATCTGAGCCGTTGGGAGGAGCTGGGAGTG
ATGGGGGTCTATGATGGCCGAGAGCATACCGTGTATGGAGAGCCAAGGAAACTGCTCACT
CAGGACCAGCGCAACACGGGCGAGATGTCCAGCAACTCCACCGCCCTCGCCCTGGTCCGA
CCTTCAAGCTCTGGCCTGATCAACTCAAATACAGATAACAACCTGGCTGTTTATGACCTC
TCTCGCTGGGAAGAACTGGGCGTTATGGGCGTGTACGATGGACGGGAGCACACAGTGTAC
GGGGAGCCCCGCAAGCTTTTAACACAAGACTGGATGGTTGAAAACAAGCTCGTGGAGTTA
GAACACACCCTGCTGAGCAAGGGGATTGAGCTGATGGAAGTGGATCCAATTGGCCACTTG
TACATCTTTGCTACCTGCCCCAGGGCACTAGCCGAGACAAGCTACGTGAAGGTGCTGGAA
CACGTGGTGCGCGTGAATGCCCGGGTCCGGATCGCTTACCCTAGCCTGAGGGAGGCAGCC
CTGCTTGAAGAGGAGGAAGGCGTCTGGCTCGAGGAGGGGCCTAGACAGTCTCTGCAACAG
TGTGTAGCAATCAGCCTTCTGACACAGTACTTCGTGCAGGAGAACTATTTAGAATATAGG
CAAGTCCCCGGG
CTA 3x Iteration Cassette AA (Standard)
ELGVMGVYDGREHTVYGEPRKELGVMGVYDGREHTVYGEPRKELGVMGVYDGREHTVYGE
PRKGIDVKEVDPTSHSYVLVTSGPRALAETSYVKVLEHVVRGPRALAETSYVKVLEYVIK
VSARVRFFFFLNMLGVYDGEEHSVFGEPWGIDVKEVDPTSHSYVLVTSGPRALAETSYVK
VLEHVVRGPRALAETSYVKVLEHVVRGPRALAETSYVKVLEYVIKVSARVRFFFFLNMLG
VYDGEEHSVFGEPWFQRNTGEMSSNSTALALVRPSSSGLINSNTDNNLAVYDLSRDSPQG
ASALPTTISFTCWRQGIELMEVDPIGHLYIFATCFQRNTGEMSSNSTALALVRPSSSGLI
NSNTDNNLAVYDLSRDSPQGASALPTTISFTCWRQGIELMEVDPIGHLYIFATCFQRNTG
EMSSNSTALALVRPSSSGLINSNTDNNLAVYDLSRDSPQGASALPTTISFTCWRQGIELM
EVDPIGHLYIFATCGPRALAETSYVKVLEYVIKVSARVRFFFFLNMLGVYDGEEHSVFGE
PWGIDVKEVDPTSHSYVLVTS
CTA 3x Iteration Cassette NT (Standard)
GAGTTAGGTGTCATGGGAGTATACGACGGTCGTGAGCATACGGTTTATGGCGAGCCGAGA
AAGGAGTTGGGCGTTATGGGGGTATATGACGGACGAGAACATACCGTTTACGGTGAGCCA
CGAAAAGAATTGGGTGTAATGGGTGTTTACGATGGTCGCGAACACACGGTATACGGAGAG
CCGCGTAAGGGTATCGACGTTAAGGAAGTGGATCCTACGAGTCATTCTTACGTATTAGTT
ACCTCTGGTCCGAGAGCGCTAGCAGAGACTAGTTATGTAAAAGTACTAGAACACGTTGTA
AGAGGACCACGGGCGCTCGCCGAGACGTCATACGTTAAAGTCCTCGAATATGTGATAAAA
GTGTCTGCGCGGGTTCGCTTTTTCTTTTTCCTAAATATGTTAGGCGTTTATGATGGGGAA
GAACATTCCGTTTTCGGGGAGCCATGGGGGATCGATGTTAAAGAGGTAGATCCCACCTCG
CATTCGTATGTCTTGGTGACGTCGGGGCCTCGTGCATTGGCGGAGACATCTTATGTTAAG
GTATTAGAGCATGTGGTTCGCGGTCCGCGTGCGCTTGCCGAAACCAGTTACGTAAAGGTC
TTGGAGCACGTCGTTCGAGGACCTCGAGCCCTGGCGGAAACAAGCTACGTGAAAGTTCTA
GAGTATGTTATTAAGGTCAGCGCTCGAGTACGGTTCTTCTTCTTCCTCAACATGCTCGGA
GTGTATGATGGCGAAGAGCATAGTGTTTTTGGAGAACCATGGTTTCAACGAAATACGGGA
GAGATGAGTAGCAACTCTACTGCCTTGGCTCTTGTGCGACCGAGTTCTTCGGGCTTGATA
AACAGCAACACGGATAATAATTTGGCCGTCTATGACTTATCACGGGATTCACCTCAAGGG
GCGTCAGCACTACCTACGACGATCTCTTTTACATGTTGGCGCCAGGGGATAGAACTCATG
GAAGTCGACCCTATTGGGCATCTTTATATTTTCGCTACATGCTTTCAAAGGAACACCGGC
GAGATGTCGAGCAATTCGACCGCGTTGGCCTTAGTACGACCATCATCTTCAGGTCTTATC
AATTCCAATACGGACAACAACCTCGCTGTCTACGATCTATCACGCGACAGTCCTCAGGGT
GCTTCGGCACTTCCTACAACAATCAGTTTCACGTGCTGGAGACAGGGAATCGAACTAATG
GAAGTTGATCCTATCGGACACTTGTATATATTTGCGACATGTTTCCAACGTAACACGGGG
GAAATGAGCTCCAACAGTACGGCTTTGGCCCTAGTTCGCCCTTCGTCGTCGGGACTCATC
AACTCAAACACCGATAACAATCTTGCTGTGTATGACCTTTCGCGCGATTCTCCACAGGGA
GCTTCAGCATTGCCAACTACTATCAGCTTCACCTGCTGGCGACAAGGGATTGAGCTTATG
GAGGTCGATCCAATCGGCCATTTGTACATCTTTGCCACCTGCGGGCCACGTGCACTCGCT
GAAACCTCGTACGTCAAAGTATTGGAATACGTAATCAAGGTCTCTGCCAGGGTTCGATTT
TTCTTCTTTCTTAATATGCTTGGGGTGTACGATGGAGAGGAGCACTCGGTATTTGGGGAA
CCTTGGGGCATAGATGTCAAGGAGGTTGACCCGACTTCACATTCATATGTACTAGTAACT
AGT
CTA 4x Iteration Cassette AA (Standard)
ELGVMGVYDGREHTVYGEPRKELGVMGVYDGREHTVYGEPRKELGVMGVYDGREHTVYGE
PRKELGVMGVYDGREHTVYGEPRKGIDVKEVDPTSHSYVLVTSGPRALAETSYVKVLEHV
VRGPRALAETSYVKVLEYVIKVSARVRFFFFLNMLGVYDGEEHSVFGEPWGIDVKEVDPT
SHSYVLVTSGPRALAETSYVKVLEHVVRGPRALAETSYVKVLEYVIKVSARVRFFFFLNM
LGVYDGEEHSVFGEPWGIDVKEVDPTSHSYVLVTSGPRALAETSYVKVLEYVIKVSARVR
FFFFLNMLGVYDGEEHSVFGEPWFQRNTGEMSSNSTALALVRPSSSGLINSNTDNNLAVY
DLSRDSPQGASALPTTISFTCWRQGIELMEVDPIGHLYIFATCFQRNTGEMSSNSTALAL
VRPSSSGLINSNTDNNLAVYDLSRDSPQGASALPTTISFTCWRQGIELMEVDPIGHLYIF
ATCFQRNTGEMSSNSTALALVRPSSSGLINSNTDNNLAVYDLSRDSPQGASALPTTISFT
CWRQGIELMEVDPIGHLYIFATCFQRNTGEMSSNSTALALVRPSSSGLINSNTDNNLAVY
DLSRDSPQGASALPTTISFTCWRQGIELMEVDPIGHLYIFATCGPRALAETSYVKVLEHV
VRGPRALAETSYVKVLEHVVRGPRALAETSYVKVLEYVIKVSARVRFFFFLNMLGVYDGE
EHSVFGEPWGIDVKEVDPTSHSYVLVTS
CTA 4x Iteration Cassette NT (Standard)
GAGTTAGGTGTGATGGGTGTATATGATGGCCGTGAACATACTGTATACGGCGAGCCTAGG
AAAGAATTGGGAGTTATGGGAGTATACGATGGTCGAGAACACACCGTTTACGGAGAACCT
AGAAAAGAGTTGGGGGTAATGGGGGTCTATGATGGTAGAGAGCATACGGTTTATGGGGAA
CCGCGAAAAGAACTTGGTGTCATGGGCGTGTATGATGGAAGGGAGCACACAGTATATGGT
GAGCCCAGAAAGGGTATTGACGTTAAAGAAGTAGATCCAACTAGTCATTCATACGTATTA
GTGACATCCGGACCTCGAGCGTTGGCAGAGACATCTTATGTAAAAGTATTAGAACACGTA
GTCAGAGGACCAAGAGCATTAGCTGAGACTAGTTATGTCAAGGTTCTTGAATACGTAATT
AAGGTGAGTGCTAGGGTACGTTTCTTCTTCTTTCTTAACATGCTAGGAGTTTATGACGGA
GAGGAGCATAGTGTCTTTGGGGAGCCGTGGGGTATCGATGTTAAGGAGGTGGATCCGACA
TCTCACTCATATGTACTGGTAACTTCTGGTCCTCGCGCACTAGCCGAGACGAGCTATGTT
AAAGTCTTGGAGCACGTTGTGAGGGGTCCAAGGGCACTGGCAGAAACGTCTTACGTTAAG
GTACTCGAATATGTGATAAAGGTATCCGCACGTGTACGGTTCTTTTTCTTCCTTAATATG
CTGGGAGTCTATGACGGGGAAGAGCACTCTGTATTCGGAGAGCCATGGGGGATTGATGTC
AAAGAGGTAGACCCAACATCCCATTCCTATGTCCTTGTTACGTCTGGACCTAGGGCGCTA
GCTGAAACGAGTTACGTGAAGGTCTTAGAGTATGTAATCAAGGTGTCAGCCAGAGTGCGA
TTTTTCTTTTTCCTAAATATGTTGGGTGTTTACGACGGTGAGGAACACAGTGTATTTGGT
GAACCGTGGTTCCAAAGGAATACGGGGGAGATGAGCTCTAATTCAACCGCACTTGCACTT
GTGAGACCGTCCTCTTCCGGGCTAATTAATAGTAACACGGATAATAATCTCGCTGTTTAT
GATTTGTCGCGTGATAGCCCACAAGGCGCTTCTGCACTACCGACCACTATATCTTTTACA
TGCTGGCGGCAAGGGATAGAACTCATGGAAGTGGACCCAATTGGACATTTGTATATTTTC
GCTACATGTTTCCAGAGAAATACAGGGGAAATGAGTAGTAACTCAACAGCTTTAGCATTG
GTCCGTCCAAGTTCATCTGGACTCATTAACTCTAACACAGACAACAATTTAGCCGTCTAC
GACTTATCAAGAGATTCTCCTCAAGGGGCGTCGGCTTTACCGACAACTATTTCATTTACC
TGCTGGAGACAGGGAATAGAATTAATGGAAGTCGACCCGATAGGACACCTTTACATATTT
GCAACGTGCTTCCAACGAAATACCGGAGAAATGTCTAGTAATTCTACCGCCTTAGCACTA
GTGCGACCTTCAAGTAGTGGTCTTATAAATTCCAATACGGACAATAATTTGGCAGTCTAC
GATTTATCTCGAGATTCACCTCAGGGGGCTTCGGCATTGCCCACTACAATTAGTTTTACT
TGTTGGAGGCAGGGTATAGAGCTTATGGAAGTTGATCCTATCGGACACTTATACATTTTT
GCTACGTGTTTTCAACGTAACACTGGAGAGATGTCTTCAAATAGTACAGCTCTGGCATTA
GTACGACCCTCTAGTTCAGGGTTAATCAATTCTAATACTGATAACAACTTGGCTGTGTAC
GATCTGTCTAGGGATTCGCCGCAAGGTGCAAGTGCATTACCTACAACGATCTCCTTCACG
TGTTGGCGTCAAGGAATTGAACTAATGGAGGTTGACCCTATTGGTCATCTTTATATATTC
GCAACCTGTGGCCCTAGAGCCCTTGCCGAAACTAGCTACGTCAAAGTTTTGGAACATGTA
GTAAGAGGCCCTCGTGCGTTAGCGGAAACTTCGTACGTAAAGGTTTTAGAGCATGTCGTT
AGAGGTCCGAGAGCGCTTGCGGAGACCTCGTATGTGAAAGTTCTAGAGTACGTTATAAAA
GTGTCTGCTAGAGTTAGATTCTTCTTCTTTTTAAACATGTTAGGCGTATATGACGGCGAA
GAACATTCTGTTTTTGGCGAACCTTGGGGAATCGACGTAAAAGAGGTCGATCCTACTTCG
CACTCCTACGTCCTAGTTACTAGT
CTA 3x Iteration Cassette AA (Full-Length)
FQRNTGEMSSNSTALALVRPSSSGLINSNTDNNLAVYDLSRDWEELGVMGVYDGREHTVY
GEPRKLLIWEFLNMLGVYDGEEHSVFGEPWKLGPRALAETSYVKVLEYVIKVSARVRFFF
LVFGIELMEVDPIGHLYIFATCLGLLLFGIDVKEVDPTSHSYVLVTSLNLLVFGIELMEV
DPIGHLYIFATCLGLLLFGIDVKEVDPTSHSYVLVTSLNLLLFGIDVKEVDPTSHSYVLV
TSLNLIWEFLNMLGVYDGEEHSVFGEPWKLGPRALAETSYVKVLEYVIKVSARVRFFFLV
FGIELMEVDPIGHLYIFATCLGLFLWGPRALAETSYVKVLEHVVRVNAPPQSPQGASALP
TTISFTCWRQPNEFLWGPRALAETSYVKVLEHVVRVNAPPQSPQGASALPTTISFTCWRQ
PNEFLWGPRALAETSYVKVLEHVVRVNAPPQSPQGASALPTTISFTCWRQPNEFQRNTGE
MSSNSTALALVRPSSSGLINSNTDNNLAVYDLSRDWEELGVMGVYDGREHTVYGEPRKLL
IWEFLNMLGVYDGEEHSVFGEPWKLFQRNTGEMSSNSTALALVRPSSSGLINSNTDNNLA
VYDLSRDWEELGVMGVYDGREHTVYGEPRKLLGPRALAETSYVKVLEYVIKVSARVRFFF
CTA 3x Iteration Cassette NT (Full-Length)
TTCCAGCGGAACACAGGAGAAATGAGCAGCAACTCAACCGCCCTTGCATTGGTCCGGCCT
TCATCATCCGGTCTCATCAATTCTAACACGGATAACAACTTGGCCGTTTATGATTTGTCG
AGAGACTGGGAAGAATTAGGCGTTATGGGCGTGTACGACGGTCGGGAGCACACGGTCTAT
GGAGAGCCCCGAAAACTGCTCATTTGGGAATTCTTAAACATGCTCGGCGTCTACGACGGC
GAGGAACATTCCGTATTTGGGGAACCTTGGAAGCTAGGGCCGCGAGCTTTGGCAGAAACC
AGTTATGTAAAAGTGCTCGAGTACGTAATAAAGGTCTCCGCTCGTGTCCGATTCTTTTTC
CTGGTTTTCGGTATAGAACTGATGGAAGTCGATCCTATCGGCCACCTCTATATATTCGCC
ACCTGTCTCGGACTGCTCCTATTTGGCATTGATGTTAAAGAGGTCGACCCGACTTCCCAT
TCCTACGTCCTTGTGACGAGCTTAAACTTGTTAGTGTTTGGTATCGAACTTATGGAGGTA
GACCCCATCGGTCATTTATACATCTTCGCGACATGTTTAGGGTTGTTGTTATTTGGAATC
GACGTCAAGGAGGTGGATCCTACCAGTCATTCATATGTACTTGTCACTTCTCTTAATCTA
CTCCTTTTCGGGATCGATGTGAAGGAAGTAGATCCCACAAGCCACAGCTATGTGCTTGTT
ACAAGCCTGAATCTGATATGGGAGTTCCTGAATATGCTAGGGGTATATGATGGAGAAGAA
CACTCAGTCTTCGGGGAGCCATGGAAATTAGGGCCACGGGCCCTGGCGGAGACCTCCTAT
GTCAAAGTCCTCGAATACGTCATCAAAGTGTCAGCCCGGGTTCGATTTTTCTTTCTCGTT
TTTGGGATTGAACTCATGGAAGTTGACCCAATCGGACACCTGTATATTTTCGCTACGTGT
CTTGGCCTCTTTTTGTGGGGTCCGCGTGCGCTGGCAGAGACGTCATACGTGAAGGTGCTA
GAGCATGTGGTAAGAGTTAACGCACCTCCGCAGTCGCCGCAAGGTGCCTCAGCGCTCCCG
ACAACGATCAGTTTTACGTGTTGGCGACAGCCGAATGAGTTCTTATGGGGGCCGAGGGCC
CTAGCCGAAACATCTTACGTTAAAGTACTAGAACATGTTGTACGCGTTAATGCACCGCCT
CAGAGTCCACAAGGGGCATCGGCTTTGCCTACGACAATTTCGTTTACATGCTGGAGGCAG
CCTAACGAATTTCTATGGGGACCGCGGGCGCTTGCGGAAACCTCTTATGTGAAAGTGTTG
GAGCACGTTGTGCGTGTCAATGCGCCACCCCAGTCTCCCCAAGGCGCGAGTGCCCTCCCT
ACAACTATCTCATTCACGTGCTGGCGCCAACCGAACGAGTTCCAAAGAAACACTGGCGAA
ATGTCATCGAATTCAACTGCCCTCGCCCTGGTCAGACCGTCGAGTTCAGGCCTCATTAAC
TCAAATACGGACAACAACCTCGCCGTATATGACCTGTCCCGCGACTGGGAGGAACTAGGC
GTAATGGGGGTGTATGACGGAAGAGAACACACCGTTTACGGAGAACCCCGCAAGTTGCTT
ATCTGGGAGTTTCTTAATATGTTGGGCGTATACGATGGCGAAGAGCACAGCGTATTCGGC
GAACCGTGGAAACTTTTCCAACGCAATACCGGAGAGATGTCGTCGAACTCTACGGCTCTC
GCTCTCGTTCGACCGAGTTCGAGCGGGCTCATAAACTCTAATACCGATAATAACCTTGCT
GTCTATGATCTATCCCGAGATTGGGAGGAGTTGGGTGTGATGGGTGTTTACGACGGACGG
GAACATACTGTTTATGGTGAGCCTCGCAAACTCCTTGGACCAAGAGCGTTAGCTGAGACA
TCGTATGTTAAGGTACTGGAATATGTAATCAAGGTCAGTGCACGAGTCCGTTTCTTCTTT
KRAS G12V & G12C “2x4” AA
MTEYKLVVVGAVGVGKSALTIQLIQMTEYKLVVVGAVGVGKSALTIQLIQMTEYKLVVVG
AVGVGKSALTIQLIQMTEYKLVVVGAVGVGKSALTIQLIQMTEYKLVVVGACGVGKSALT
IQLIQMTEYKLVVVGACGVGKSALTIQLIQMTEYKLVVVGACGVGKSALTIQLIQMTEYK
LVVVGACGVGKSALTIQLIQ
KRAS G12V & G12C “2x4” NT
ATGACAGAATATAAGTTGGTCGTAGTAGGAGCCGTAGGTGTTGGTAAATCTGCGCTAACT
ATTCAACTCATTCAGATGACTGAGTATAAACTTGTTGTAGTGGGAGCAGTCGGTGTGGGC
AAAAGTGCGCTTACAATCCAATTAATCCAAATGACGGAGTATAAGCTAGTAGTAGTCGGA
GCGGTTGGTGTCGGAAAGTCAGCTTTAACTATACAGCTTATCCAGATGACCGAATACAAA
CTAGTTGTCGTGGGTGCGGTCGGCGTAGGGAAATCAGCTCTAACGATACAATTGATACAA
ATGACCGAGTACAAGCTTGTAGTCGTCGGTGCATGCGGGGTTGGCAAATCGGCGTTGACC
ATCCAACTTATACAAATGACTGAATACAAGTTAGTGGTTGTCGGCGCTTGCGGTGTAGGA
AAGAGTGCACTTACGATCCAGTTAATACAGATGACGGAATATAAATTAGTCGTGGTTGGA
GCGTGTGGCGTGGGTAAAAGCGCATTAACGATTCAGTTGATTCAAATGACAGAGTACAAA
TTGGTGGTAGTTGGTGCGTGCGGAGTCGGTAAGTCGGCTCTCACAATACAACTAATTCAG
GGACCAGGCCCAGGCGCTAAGTTCGTAGCTGCATGGACACTGAAAGCCGCTGCTGGTCCA
GGACCCGGACAGTATATTAAGGCTAATAGTAAGTTTATAGGCATAACTGAATTAGGCCCT
GGGCCCGGGTGATAGTAA
KRAS 4X (4x4) AA
MTEYKLVVVGACGVGKSALTIQLIQMTEYKLVVVGADGVGKSALTIQLIQETCLLDILDT
AGHEEYSAMRDQYMRMTEYKLVVVGADGVGKSALTIQLIQMTEYKLVVVGAVGVGKSALT
IQLIQMTEYKLVVVGACGVGKSALTIQLIQETCLLDILDTAGHEEYSAMRDQYMRMTEYK
LVVVGADGVGKSALTIQLIQMTEYKLVVVGAVGVGKSALTIQLIQMTEYKLVVVGACGVG
KSALTIQLIQETCLLDILDTAGHEEYSAMRDQYMRMTEYKLVVVGADGVGKSALTIQLIQ
MTEYKLVVVGAVGVGKSALTIQLIQETCLLDILDTAGHEEYSAMRDQYMRMTEYKLVVVG
AVGVGKSALTIQLIQMTEYKLVVVGACGVGKSALTIQLIQ
KRAS 4X (4x4) NT
ATGACCGAGTATAAACTAGTAGTTGTGGGAGCGTGTGGTGTAGGCAAGTCGGCACTTACA
ATTCAGTTGATACAAATGACGGAATATAAGCTCGTAGTAGTCGGAGCAGACGGCGTGGGG
AAATCAGCGTTGACTATCCAGTTAATACAGGAAACTTGCCTATTAGACATCTTGGATACG
GCAGGTCATGAGGAATATTCCGCTATGAGAGATCAGTATATGCGCATGACGGAGTATAAG
CTTGTGGTTGTCGGGGCCGACGGGGTAGGTAAGTCAGCGCTCACGATACAATTAATTCAA
ATGACCGAATACAAGTTGGTCGTGGTGGGGGCAGTTGGGGTCGGTAAATCCGCGTTAACG
ATCCAACTTATCCAAATGACAGAATATAAACTCGTTGTTGTAGGTGCATGTGGCGTAGGA
AAAAGCGCATTGACCATCCAGCTAATTCAGGAGACGTGTCTCCTTGATATCCTAGACACG
GCGGGGCACGAAGAATACTCGGCTATGCGCGACCAGTACATGAGAATGACGGAATACAAA
CTTGTTGTCGTGGGTGCGGATGGAGTAGGGAAAAGTGCTCTAACAATACAACTCATTCAG
ATGACAGAGTACAAATTGGTAGTCGTCGGTGCGGTAGGAGTTGGGAAGTCTGCACTAACT
ATTCAGCTCATACAGATGACCGAGTACAAGCTGGTGGTGGTAGGCGCTTGCGGTGTGGGT
AAGAGTGCATTAACCATACAGCTTATACAAGAGACATGTCTGCTAGATATATTAGATACC
GCCGGGCATGAAGAGTACTCTGCCATGCGAGACCAATACATGCGTATGACAGAGTATAAA
TTAGTAGTGGTTGGGGCGGACGGTGTTGGCAAGAGCGCCTTAACTATACAGTTGATCCAG
ATGACGGAGTACAAACTGGTCGTCGTTGGTGCAGTGGGAGTGGGAAAATCTGCGCTGACG
ATTCAACTAATCCAAGAAACATGTTTACTTGACATCCTCGACACTGCGGGTCACGAGGAG
TATTCGGCGATGCGTGATCAATATATGAGGATGACTGAGTATAAGTTAGTCGTAGTTGGA
GCGGTCGGTGTCGGAAAGTCCGCGCTAACCATTCAATTGATTCAAATGACTGAATACAAG
CTAGTGGTAGTAGGAGCATGCGGCGTCGGCAAATCGGCTTTAACAATCCAACTGATACAG
CTA8x2 KRAS2x4 Cassette AA
MTEYKLVVVGAVGVGKSALTIQLIIDVKEVDPTSHSYVLVTSLPRALAETSYVKVLEHVV
RVNARVPRALAETSYVKVLEYVIKVSARVRFFLNMLGVYDGEEHSVFGEPWMTEYKLVVV
GAVGVGKSALTIQLIIDVKEVDPTSHSYVLVTSLPRALAETSYVKVLEYVIKVSARVRFF
LNMLGVYDGEEHSVFGEPWMTEYKLVVVGACGVGKSALTIQLIQRNTGEMSSNSTALALV
RPSSSGLINSNTDNNLAVYDLSRWEELGVMGVYDGREHTVYGEPRKMTEYKLVVVGAVGV
GKSALTIQLIIELMEVDPIGHLYIFATCMTEYKLVVVGACGVGKSALTIQLIQSPQGASA
LPTTISFTCWRIELMEVDPIGHLYIFATCMTEYKLVVVGACGVGKSALTIQLIQRNTGEM
SSNSTALALVRPSSSGLINSNTDNNLAVYDLSRWEELGVMGVYDGREHTVYGEPRKMTEY
KLVVVGAVGVGKSALTIQLIQSPQGASALPTTISFTCWRPRALAETSYVKVLEHVVRVNA
RVRIAYMTEYKLVVVGACGVGKSALTIQLIQ
CTA8x2 KRAS2x4 Cassette NT
ATGACAGAGTATAAGTTGGTTGTAGTGGGTGCCGTAGGCGTGGGAAAATCAGCTCTGACC
ATTCAGTTGATAATAGACGTAAAAGAGGTTGACCCCACTAGTCATTCGTACGTTTTAGTT
ACTAGTTTACCAAGGGCTCTCGCCGAAACATCATACGTCAAGGTCCTGGAACACGTTGTT
CGCGTTAATGCGCGAGTTCCGAGAGCATTAGCTGAGACATCCTATGTGAAAGTACTCGAG
TATGTGATCAAAGTTTCCGCCCGCGTCAGATTTTTCCTTAATATGCTCGGAGTTTATGAT
GGTGAGGAACATTCGGTGTTTGGCGAGCCTTGGATGACTGAATACAAACTTGTCGTGGTC
GGGGCCGTTGGAGTCGGAAAGAGCGCTTTGACCATCCAGTTAATTATAGATGTCAAAGAA
GTGGATCCGACCTCACACTCGTATGTATTGGTCACGTCCCTACCACGCGCGTTGGCAGAA
ACCAGTTATGTTAAGGTCTTAGAGTACGTCATAAAAGTATCCGCCAGAGTTAGATTCTTC
CTAAACATGCTTGGGGTGTACGACGGAGAAGAACACAGTGTTTTCGGAGAGCCATGGATG
ACGGAGTACAAATTAGTCGTCGTAGGGGCATGCGGAGTGGGCAAATCCGCCCTCACCATA
CAGCTCATACAACGCAACACCGGGGAAATGTCTTCAAACTCAACCGCGCTTGCGCTGGTT
CGGCCAAGCAGCAGCGGGCTAATCAACAGCAATACGGATAATAACTTAGCCGTATATGAC
CTCAGTCGGTGGGAGGAATTAGGGGTCATGGGTGTCTATGACGGTCGAGAACATACGGTA
TATGGGGAGCCACGGAAGATGACCGAGTACAAGCTAGTTGTTGTTGGGGCAGTGGGAGTT
GGGAAATCTGCCCTTACAATTCAGCTGATCATCGAACTCATGGAAGTTGATCCCATCGGA
CATTTGTACATCTTTGCCACTTGTATGACCGAATATAAATTGGTGGTCGTCGGTGCCTGC
GGTGTAGGTAAGTCCGCTTTAACTATACAACTTATACAATCTCCTCAGGGTGCTAGCGCA
CTTCCGACGACTATTAGTTTTACCTGCTGGAGGATTGAGTTGATGGAGGTCGATCCAATA
GGTCACCTATATATCTTCGCAACATGCATGACGGAATACAAGTTAGTGGTAGTCGGAGCT
TGCGGCGTAGGAAAGTCAGCCCTGACTATCCAATTAATCCAGCGTAATACCGGAGAGATG
TCGTCAAATAGTACAGCCTTAGCATTGGTTCGCCCGTCGTCCAGCGGTTTAATCAATTCC
AACACGGACAATAACCTGGCGGTCTACGATCTATCGCGCTGGGAAGAACTAGGGGTTATG
GGGGTTTACGATGGTCGCGAGCATACCGTATACGGGGAACCGAGAAAAATGACTGAGTAT
AAACTCGTCGTTGTCGGCGCGGTTGGGGTTGGAAAAAGCGCATTGACGATTCAATTGATT
CAGTCCCCACAGGGGGCTTCTGCTCTACCGACAACCATCAGCTTTACATGTTGGAGGCCT
AGAGCTCTGGCAGAGACTTCTTATGTAAAAGTTCTCGAGCATGTCGTTCGGGTGAACGCA
AGAGTCAGAATTGCTTACATGACAGAATATAAGCTGGTGGTTGTGGGAGCCTGTGGGGTC
GGCAAGTCTGCACTGACGATCCAGCTTATTCAA
CTA8x2 “Option 1”-T2A-2x4KRAS AA: T2A italics
ELGVMGVYDGREHTVYGEPRKELGVMGVYDGREHTVYGEPRKGIDVKEVDPTSHSYVLVT
SGPRALAETSYVKVLEHVVRVNARVRGPRALAETSYVKVLEYVIKVSARVRFFFFLNMLG
VYDGEEHSVFGEPWFQRNTGEMSSNSTALALVRPSSSGLINSNTDNNLAVYDLSRDSPQG
ASALPTTISFTCWRQGIELMEVDPIGHLYIFATCFQRNTGEMSSNSTALALVRPSSSGLI
NSNTDNNLAVYDLSRDSPQGASALPTTISFTCWRQGIELMEVDPIGHLYIFATCGPRALA
ETSYVKVLEHVVRVNARVRGPRALAETSYVKVLEYVIKVSARVRFFFFLNMLGVYDGEEH
SVFGEPWGIDVKEVDPTSHSYVLVTSGGSGGVRAEGRGSLLTCGDVEENPGPMAGMTEYK
LVVVGAVGVGKSALTIQLIMTEYKLVVVGAVGVGKSALTIQLIMTEYKLVVVGAVGVGKS
ALTIQLIMTEYKLVVVGAVGVGKSALTIQLIMTEYKLVVVGACGVGKSALTIQLIMTEYK
LVVVGACGVGKSALTIQLIMTEYKLVVVGACGVGKSALTIQLIMTEYKLVVVGACGVGKS
ALTIQLIQ
CTA8x2 “Option 1”-T2A-2x4KRAS NT: T2A italics;
KRAS Bold
GAACTTGGTGTCATGGGTGTCTACGATGGGAGAGAACACACGGTATACGGCGAGCCACGA
AAGGAACTCGGCGTTATGGGCGTGTATGACGGACGAGAGCACACAGTCTACGGAGAGCCG
CGCAAAGGAATAGACGTCAAGGAGGTAGACCCGACGTCACACTCTTACGTGCTCGTTACC
TCAGGTCCCCGAGCCTTGGCGGAGACATCGTATGTCAAAGTCTTGGAGCACGTTGTTCGT
GTGAACGCTCGGGTTAGAGGGCCACGAGCGCTCGCGGAAACCAGCTATGTAAAGGTGTTG
GAATACGTCATCAAAGTTTCCGCGAGGGTTCGATTTTTCTTCTTCCTCAATATGCTCGGT
GTATACGACGGGGAAGAGCACTCTGTATTCGGGGAGCCATGGTTTCAACGGAACACTGGA
GAGATGTCCTCCAATTCTACAGCTTTGGCTCTGGTGCGACCCAGTTCCTCAGGACTCATC
AATTCCAACACGGATAATAATTTGGCAGTTTACGATCTCAGCAGGGACTCTCCACAGGGA
GCGTCAGCGCTCCCCACGACTATCAGTTTTACCTGCTGGCGACAAGGCATCGAACTCATG
GAAGTGGACCCGATTGGTCATCTTTATATATTCGCGACTTGCTTTCAGAGGAATACCGGG
GAAATGTCTTCCAACTCCACGGCGCTGGCATTGGTCCGGCCCAGCAGCTCCGGTCTTATC
AACTCAAACACAGACAATAACTTGGCCGTATATGATCTATCTCGGGACAGCCCACAAGGA
GCTTCTGCACTGCCGACCACTATTAGCTTCACCTGTTGGCGGCAGGGAATCGAGTTGATG
GAGGTTGATCCTATAGGCCACTTATACATTTTTGCCACATGCGGACCAAGAGCGCTTGCC
GAAACATCCTATGTGAAGGTGCTCGAACATGTTGTACGAGTCAATGCTAGAGTCAGGGGG
CCCCGGGCCCTCGCAGAAACTTCCTACGTGAAAGTCCTGGAGTACGTTATCAAGGTGTCA
GCACGAGTACGCTTCTTTTTCTTTCTAAACATGCTGGGAGTTTATGATGGGGAGGAACAT
TCGGTTTTCGGCGAACCGTGGGGGATCGATGTAAAAGAGGTGGATCCGACTTCCCACTCG
TACGTCTTGGTTACGTCGggcggtagcggcGGGGTTCGGGCAGAGGGCAGAGGCTCACTG
CTCACGTGTGGTGATGTAGAAGAGAATCCAGGTCCTATGGCTGGCATGACGGAATATAAA
TTAGTTGTGGTTGGTGCCGTCGGTGTTGGTAAGAGTGCCCTCACAATCCAATTAATTATG
ACTGAATACAAGCTGGTGGTAGTAGGGGCGGTGGGAGTAGGCAAGTCAGCATTAACCATT
CAGCTCATAATGACTGAGTACAAGTTGGTCGTAGTGGGTGCAGTCGGCGTCGGGAAATCG
GCCCTGACGATTCAGCTGATTATGACAGAATACAAACTCGTTGTTGTCGGAGCTGTCGGG
GTTGGGAAGTCTGCTCTTACTATTCAGTTAATTATGACCGAGTACAAATTGGTTGTTGTG
GGAGCCTGTGGCGTTGGCAAAAGCGCTTTGACTATCCAACTCATCATGACAGAGTATAAA
CTGGTGGTGGTCGGAGCATGCGGAGTGGGCAAATCTGCGCTGACTATACAACTCATTATG
ACGGAGTATAAGTTAGTAGTCGTTGGGGCTTGTGGGGTCGGAAAGAGTGCGCTTACCATA
CAGTTAATAATGACCGAATATAAGCTAGTGGTAGTTGGCGCGTGTGGTGTCGGTAAGTCC
GCGTTAACAATTCAACTGATACAG
ChAd CMV-CTA8x2-EFT-KRAS-4x4 (Dual Promoter):
vector elements in order-CMV enhancer/promoter;
CTA8x2; PADRE/TT Class II + GPGPG linkers;
SV40 Poly A; TETo regulated EF1alpha promoter; KRAS2x4;
PADRE/TT Class II + GPGPG linkers; BGH poly A
GACATTGATTATTGACTAGTTGTTAATAGTAATCAATTACGGGGTCATTAGTTCATAGCC
CATATATGGAGTTCCGCGTTACATAACTTACGGTAAATGGCCCGCCTGGCTGACCGCCCA
ACGACCCCCGCCCATTGACGTCAATAATGACGTATGTTCCCATAGTAACGCCAATAGGGA
CTTTCCATTGACGTCAATGGGTGGAGTATTTACGGTAAACTGCCCACTTGGCAGTACATC
AAGTGTATCATATGCCAAGTCCGCCCCCTATTGACGTCAATGACGGTAAATGGCCCGCCT
GGCATTATGCCCAGTACATGACCTTACGGGACTTTCCTACTTGGCAGTACATCTACGTAT
TAGTCATCGCTATTACCATGGTGATGCGGTTTTGGCAGTACACCAATGGGCGTGGATAGC
GGTTTGACTCACGGGGATTTCCAAGTCTCCACCCCATTGACGTCAATGGGAGTTTGTTTT
GGCACCAAAATCAACGGGACTTTCCAAAATGTCGTAATAACCCCGCCCCGTTGACGCAAA
TGGGCGGTAGGCGTGTACGGTGGGAGGTCTATATAAGCAGAGCTCGTTTAGTGAACCGTC
AGATCgcctggaacgccatccacgctgttttgacctccatagaagacagegatCGCGCCA
CCATGGCTGGCGAACTTGGTGTCATGGGTGTCTACGATGGGAGAGAACACACGGTATACG
GCGAGCCACGAAAGGAACTCGGCGTTATGGGCGTGTATGACGGACGAGAGCACACAGTCT
ACGGAGAGCCGCGCAAAGGAATAGACGTCAAGGAGGTAGACCCGACGTCACACTCTTACG
TGCTCGTTACCTCAGGTCCCCGAGCCTTGGCGGAGACATCGTATGTCAAAGTCTTGGAGC
ACGTTGTTCGTGTGAACGCTCGGGTTAGAGGGCCACGAGCGCTCGCGGAAACCAGCTATG
TAAAGGTGTTGGAATACGTCATCAAAGTTTCCGCGAGGGTTCGATTTTTCTTCTTCCTCA
ATATGCTCGGTGTATACGACGGGGAAGAGCACTCTGTATTCGGGGAGCCATGGTTTCAAC
GGAACACTGGAGAGATGTCCTCCAATTCTACAGCTTTGGCTCTGGTGCGACCCAGTTCCT
CAGGACTCATCAATTCCAACACGGATAATAATTTGGCAGTTTACGATCTCAGCAGGGACT
CTCCACAGGGAGCGTCAGCGCTCCCCACGACTATCAGTTTTACCTGCTGGCGACAAGGCA
TCGAACTCATGGAAGTGGACCCGATTGGTCATCTTTATATATTCGCGACTTGCTTTCAGA
GGAATACCGGGGAAATGTCTTCCAACTCCACGGCGCTGGCATTGGTCCGGCCCAGCAGCT
CCGGTCTTATCAACTCAAACACAGACAATAACTTGGCCGTATATGATCTATCTCGGGACA
GCCCACAAGGAGCTTCTGCACTGCCGACCACTATTAGCTTCACCTGTTGGCGGCAGGGAA
TCGAGTTGATGGAGGTTGATCCTATAGGCCACTTATACATTTTTGCCACATGCGGACCAA
GAGCGCTTGCCGAAACATCCTATGTGAAGGTGCTCGAACATGTTGTACGAGTCAATGCTA
GAGTCAGGGGGCCCCGGGCCCTCGCAGAAACTTCCTACGTGAAAGTCCTGGAGTACGTTA
TCAAGGTGTCAGCACGAGTACGCTTCTTTTTCTTTCTAAACATGCTGGGAGTTTATGATG
GGGAGGAACATTCGGTTTTCGGCGAACCGTGGGGGATCGATGTAAAAGAGGTGGATCCGA
CTTCCCACTCGTACGTCTTGGTTACGTCGGGACCCGGACCAGGCGCCAAATTTGTTGCTG
CTTGGACACTGAAAGCTGCTGCTGGGCCCGGACCAGGCCAGTACATCAAGGCCAACTCTA
AGTTTATCGGCATCACCGAATTGGGACCTGGACCCGGCTAGTGAGTTAAACTCCCATTTA
AATGTGAGGGTTAATGCTTCGAGCAGACATGATAAGATACATTGATGAGTTTGGACAAAC
CACAACTAGAATGCAGTGAAAAAAATGCTTTATTTGTGAAATTTGTGATGCTATTGCTTT
ATTTGTAACCATTATAAGCTGCAATAAACAAGTTAACAACAACAATTGCATTCATTTTAT
GTTTCAGGTTCAGGGGGAGATGTGGGAGGTTTTTTAAAGCAAGTAAAACCTCTACAAATG
TGGTAAAATAACTATAACGGTCCTAAGGTAGCGAGGCTCCGGTGCCCGTCAGTGGGCAGA
GCGCACATCGCCCACAGTCCCCGAGAAGTTGGGGGGAGGGGTCGGCAATTGAACCGGTGC
CTAGAGAAGGTGGCGCGGGGTAAACTGGGAAAGTGATGTCGTGTACTGGCTCCGCCTTTT
TCCCGAGGGTGGGGGAGAACCGTATATAAGTGCAGTAGtccctatcagtgatagagatct
ccctatcagtgatagagaTCGCCGTGAACGTTCTTTTTCGCAACGGGTTTGCCGCCAGAA
CACAGGTAAGTGCCGTGTGTGGTTCCCGCGGGCCTGGCCTCTTTACGGGTTATGGCCCTT
GCGTGCCTTGAATTACTTCCACCTGGCTGCAGTACGTGATTCTTGATCCCGAGCTTCGGG
TTGGAAGTGGGTGGGAGAGTTCGAGGCCTTGCGCTTAAGGAGCCCCTTCGCCTCGTGCTT
GAGTTGAGGCCTGGCCTGGGCGCTGGGGCCGCCGCGTGCGAATCTGGTGGCACCTTCGCG
CCTGTCTCGCTGCTTTCGATAAGTCTCTAGCCATTTAAAATTTTTGATGACCTGCTGCGA
CGCTTTTTTTCTGGCAAGATAGTCTTGTAAATGCGGGCCAAGATCTGCACACTGGTATTT
CGGTTTTTGGGGCCGCGGGCGGCGACGGGGCCCGTGCGTCCCAGCGCACATGTTCGGCGA
GGCGGGGCCTGCGAGCGCGGCCACCGAGAATCGGACGGGGGTAGTCTCAAGCTGGCCGGC
CTGCTCTGGTGCCTGGCCTCGCGCCGCCGTGTATCGCCCCGCCCTGGGCGGCAAGGCTGG
CCCGGTCGGCACCAGTTGCGTGAGCGGAAAGATGGCCGCTTCCCGGCCCTGCTGCAGGGA
GCTCAAAATGGAGGACGCGGCGCTCGGGAGAGCGGGCGGGTGAGTCACCCACACAAAGGA
AAAGGGCCTTTCCGTCCTCAGCCGTCGCTTCATGTGACTCCACGGAGTACCGGGCGCCGT
CCAGGCACCTCGATTAGTTCTCGAGCTTTTGGAGTACGTCGTCTTTAGGTTGGGGGGAGG
GGTTTTATGCGATGGAGTTTCCCCACACTGAGTGGGTGGAGACTGAAGTTAGGCCAGCTT
GGCACTTGATGTAATTCTCCTTGGAATTTGCCCTTTTTGAGTTTGGATCTTGGTTCATTC
TCAAGCCTCAGACAGTGGTTCAAAGTTTTTTTCTTCCATTTCAGGTGTCGTGAtgaagtg
gtggaaaccgcattctgtactttcgtgctgtcgcggGCGATCGCGCCACCATGGCTGGCA
TGACGGAATATAAATTAGTTGTGGTTGGTGCCGTCGGTGTTGGTAAGAGTGCCCTCACAA
TCCAATTAATTATGACTGAATACAAGCTGGTGGTAGTAGGGGCGGTGGGAGTAGGCAAGT
CAGCATTAACCATTCAGCTCATAATGACTGAGTACAAGTTGGTCGTAGTGGGTGCAGTCG
GCGTCGGGAAATCGGCCCTGACGATTCAGCTGATTATGACAGAATACAAACTCGTTGTTG
TCGGAGCTGTCGGGGTTGGGAAGTCTGCTCTTACTATTCAGTTAATTATGACCGAGTACA
AATTGGTTGTTGTGGGAGCCTGTGGCGTTGGCAAAAGCGCTTTGACTATCCAACTCATCA
TGACAGAGTATAAACTGGTGGTGGTCGGAGCATGCGGAGTGGGCAAATCTGCGCTGACTA
TACAACTCATTATGACGGAGTATAAGTTAGTAGTCGTTGGGGCTTGTGGGGTCGGAAAGA
GTGCGCTTACCATACAGTTAATAATGACCGAATATAAGCTAGTGGTAGTTGGCGCGTGTG
GTGTCGGTAAGTCCGCGTTAACAATTCAACTGATACAGGGACCAGGCCCAGGCGCTAAGT
TCGTAGCTGCATGGACACTGAAAGCCGCTGCTGGTCCAGGACCCGGACAGTATATTAAGG
CTAATAGTAAGTTTATAGGCATAACTGAATTAGGCCCTGGGCCCGGGTGATGAGTTTAAA
CaaaaagcaccgggataacacgctcaccatgaagAGAGCTCGCTGATCAGCCTCGACTGT
GCCTTCTAGTTGCCAGCCATCTGTTGTTTGCCCCTCCCCCGTGCCTTCCTTGACCCTGGA
AGGTGCCACTCCCACTGTCCTTTCCTAATAAAATGAGGAAATTGCATCGCATTGTCTGAG
TAGGTGTCATTCTATTCTGGGGGGTGGGGTGGGGCAGGACAGCAAGGGGGAGGATTGGGA
AGACAATAGCAGGCATGCTGGGGATGCGGTGGGCTCTATGGCTTCTGAGGCGGAAAGAAC
C
samRNA-26S-CTA8x2-26XV2-KRAS2x4 (Dual Promoter):
vector elements in order-26SV1; CTA8x2;
PADRE/TT Class II + GPGPG linkers;
26SV2; KRAS2x4; PADRE/TT Class II + GPGPG linkers
ctctctacggctaacctgaatggactacgactctagaatagtctttaattaagccaccAT
GGCTGGCGAACTTGGTGTCATGGGTGTCTACGATGGGAGAGAACACACGGTATACGGCGA
GCCACGAAAGGAACTCGGCGTTATGGGCGTGTATGACGGACGAGAGCACACAGTCTACGG
AGAGCCGCGCAAAGGAATAGACGTCAAGGAGGTAGACCCGACGTCACACTCTTACGTGCT
CGTTACCTCAGGTCCCCGAGCCTTGGCGGAGACATCGTATGTCAAAGTCTTGGAGCACGT
TGTTCGTGTGAACGCTCGGGTTAGAGGGCCACGAGCGCTCGCGGAAACCAGCTATGTAAA
GGTGTTGGAATACGTCATCAAAGTTTCCGCGAGGGTTCGATTTTTCTTCTTCCTCAATAT
GCTCGGTGTATACGACGGGGAAGAGCACTCTGTATTCGGGGAGCCATGGTTTCAACGGAA
CACTGGAGAGATGTCCTCCAATTCTACAGCTTTGGCTCTGGTGCGACCCAGTTCCTCAGG
ACTCATCAATTCCAACACGGATAATAATTTGGCAGTTTACGATCTCAGCAGGGACTCTCC
ACAGGGAGCGTCAGCGCTCCCCACGACTATCAGTTTTACCTGCTGGCGACAAGGCATCGA
ACTCATGGAAGTGGACCCGATTGGTCATCTTTATATATTCGCGACTTGCTTTCAGAGGAA
TACCGGGGAAATGTCTTCCAACTCCACGGCGCTGGCATTGGTCCGGCCCAGCAGCTCCGG
TCTTATCAACTCAAACACAGACAATAACTTGGCCGTATATGATCTATCTCGGGACAGCCC
ACAAGGAGCTTCTGCACTGCCGACCACTATTAGCTTCACCTGTTGGCGGCAGGGAATCGA
GTTGATGGAGGTTGATCCTATAGGCCACTTATACATTTTTGCCACATGCGGACCAAGAGC
GCTTGCCGAAACATCCTATGTGAAGGTGCTCGAACATGTTGTACGAGTCAATGCTAGAGT
CAGGGGGCCCCGGGCCCTCGCAGAAACTTCCTACGTGAAAGTCCTGGAGTACGTTATCAA
GGTGTCAGCACGAGTACGCTTCTTTTTCTTTCTAAACATGCTGGGAGTTTATGATGGGGA
GGAACATTCGGTTTTCGGCGAACCGTGGGGGATCGATGTAAAAGAGGTGGATCCGACTTC
CCACTCGTACGTCTTGGTTACGTCGGGACCCGGACCAGGCGCCAAATTTGTTGCTGCTTG
GACACTGAAAGCTGCTGCTGGGCCCGGACCAGGCCAGTACATCAAGGCCAACTCTAAGTT
TATCGGCATCACCGAATTGGGACCTGGACCCGGCTAGTGAAttcgaattgggcccctata
actctctacggctaacctgaatggactacgacatagtctagtccgccaagGCCACCATGG
CTGGCATGACGGAATATAAATTAGTTGTGGTTGGTGCCGTCGGTGTTGGTAAGAGTGCCC
TCACAATCCAATTAATTATGACTGAATACAAGCTGGTGGTAGTAGGGGCGGTGGGAGTAG
GCAAGTCAGCATTAACCATTCAGCTCATAATGACTGAGTACAAGTTGGTCGTAGTGGGTG
CAGTCGGCGTCGGGAAATCGGCCCTGACGATTCAGCTGATTATGACAGAATACAAACTCG
TTGTTGTCGGAGCTGTCGGGGTTGGGAAGTCTGCTCTTACTATTCAGTTAATTATGACCG
AGTACAAATTGGTTGTTGTGGGAGCCTGTGGCGTTGGCAAAAGCGCTTTGACTATCCAAC
TCATCATGACAGAGTATAAACTGGTGGTGGTCGGAGCATGCGGAGTGGGCAAATCTGCGC
TGACTATACAACTCATTATGACGGAGTATAAGTTAGTAGTCGTTGGGGCTTGTGGGGTCG
GAAAGAGTGCGCTTACCATACAGTTAATAATGACCGAATATAAGCTAGTGGTAGTTGGCG
CGTGTGGTGTCGGTAAGTCCGCGTTAACAATTCAACTGATACAGGGACCAGGCCCAGGCG
CTAAGTTCGTAGCTGCATGGACACTGAAAGCCGCTGCTGGTCCAGGACCCGGACAGTATA
TTAAGGCTAATAGTAAGTTTATAGGCATAACTGAATTAGGCCCTGGGCCCGGGTGATAGT
AA

### Adenoviral Vectors

A modified ChAdV68 vector for the antigen expression system was generated based on AC_000011.1 with E1 (nt 577 to 3403) and E3 (nt 27,125-31,825) sequences deleted and the corresponding ATCC VR-594 (Independently sequenced Full-Length VR-594 C68 SEQ ID NO: 10) nucleotides substituted at five positions. The full-length ChAdVC68 AC_000011.1 sequence with corresponding ATCC VR-594 nucleotides substituted at five positions is referred to as “ChAdV68.5WTnt” (SEQ TD NO: 1). Antigen cassettes under the control of the CMV promoter/enhancer were inserted in place of the deleted E1 sequences. A representative ChAdV68 vector containing 20 model antigens in an antigen cassette is “ChAdV68.5WTnt.MAG25mer” (SEQ ID NO:2). The vectors featuring antigen cassettes described below having the MAG25mer cassette were replaced by the antigen cassettes described above, e.g., in Table 2B.

An additional modified ChAdV68 vector (“ChAdV68-Empty-E4deleted” SEQ ID NO:10,758) for the antigen expression system was generated based on AC_000011.1 with E1 (nt 577 to 3403), E3 (nt 27,125-31,825), and E4 region (nt 34,916 to 35,642) sequences deleted and the corresponding ATCC VR-594 (Independently sequenced Full-Length VR-594 C68 SEQ ID NO:10) nucleotides substituted at five positions. The full-length ChAdV68 AC_000011.1 sequence with corresponding ATCC VR-594 nucleotides substituted at five positions is referred to as “ChAdV68.5WTnt” (SEQ ID NO:1). Antigen cassettes (e.g., in Table 2B) under the control of a CMV promoter/enhancer including a TET response region (“CMT”) were inserted in place of the deleted E1 sequences. The TET response region includes two repeats of the 19 bp TET operator (TETo) sequence (TCCCTATCAGTGATAGAGA) linked together with a two nucleotide spacer. The TET response region is downstream (3′) of a full-length CMV promoter and upstream (5′) of the start of the cassette location.

### Adenoviral Production in 293F Cells

ChAdV68 virus production was performed in 293F cells grown in 293 FreeStyle™ (ThermoFisher) media in an incubator at 8% CO₂. On the day of infection cells were diluted to 10⁶ cells per mL, with 98% viability and 400 mL are used per production run in 1L Shake flasks (Corning). 4 mL of the tertiary viral stock with a target MOI of >3.3 was used per infection. The cells were incubated for 48-72 h until the viability was <70% as measured by Trypan blue. The infected cells were then harvested by centrifugation, full speed bench top centrifuge and washed in 1×PBS, re-centrifuged and then re-suspended in 20 mL of 10 mM Tris pH7.4. The cell pellet was lysed by freeze thawing 3× and clarified by centrifugation at 4,300×g for 5 minutes.

Adenoviral Production in 293F Cells with Regulated Promoter

A TETr-regulated viral expression system was established to minimize transcription of nucleic acids encoded in a cassette, such as an antigen encoding cassette in a vaccine, during viral production. The general strategy follows:
293F cells expressing a TET repressor protein (TETr) repress expression of the vaccine cassette by binding to the TET operator sequence upstream of a minimal CMV promoter
Transcription of the cassette sequence facilitates Adenovirus production without the influence of cassette expression
Once administered in vivo, no repressor is present, and transcription of the cassette can proceed unimpeded
   Adenoviral Purification by CsCl centrifugation

Viral DNA was purified by CsCl centrifugation. Two discontinuous gradient runs were performed. The first to purify virus from cellular components and the second to further refine separation from cellular components and separate defective from infectious particles.

10 mL of 1.2 (26.8 g CsCl dissolved in 92 mL of 10 mM Tris pH 8.0) CsCl was added to polyallomer tubes. Then 8 mL of 1.4 CsCl (53 g CsCl dissolved in 87 mL of 10 mM Tris pH 8.0) was carefully added using a pipette delivering to the bottom of the tube. The clarified virus was carefully layered on top of the 1.2 layer. If needed more 10 mM Tris was added to balance the tubes. The tubes were then placed in a SW-32Ti rotor and centrifuged for 2 h 30 min at 10° C. The tube was then removed to a laminar flow cabinet and the virus band pulled using an 18 gauge needle and a 10 mL syringe. Care was taken not to remove contaminating host cell DNA and protein. The band was then diluted at least 2× with 10 mM Tris pH 8.0 and layered as before on a discontinuous gradient as described above. The run was performed as described before except that this time the run was performed overnight. The next day the band was pulled with care to avoid pulling any of the defective particle band. The virus was then dialyzed using a Slide-a-Lyzer™ Cassette (Pierce) against ARM buffer (20 mM Tris pH 8.0, 25 mM NaCl, 2.5% Glycerol). This was performed 3×, 1 h per buffer exchange. The virus was then aliquoted for storage at −80° C.

### Adenoviral Viral Assays

VP concentration was performed by using an OD 260 assay based on the extinction coefficient of 1.1×10¹² viral particles (VP) was equivalent to an Absorbance value of 1 at OD260 nm. Two dilutions (1:5 and 1:10) of adenovirus were made in a viral lysis buffer (0.1% SDS, 10 mM Tris pH 7.4, 1 mM EDTA). OD was measured in duplicate at both dilutions and the VP concentration/mL was measured by multiplying the OD260 value×dilution factor×1.1×10¹²VP.

An infectious unit (IU) titer was calculated by a limiting dilution assay of the viral stock. The virus was initially diluted 100× in DMEM/5% NS/1×PS and then subsequently diluted using 10-fold dilutions down to 1×10⁻⁷. 100 μL of these dilutions were then added to 293A cells that were seeded at least an hour before at 3e5 cells/well of a 24 well plate. This was performed in duplicate. Plates were incubated for 48 h in a CO₂ (5%) incubator at 37° C. The cells were then washed with 1×PBS and were then fixed with 100% cold methanol (−20° C.). The plates were then incubated at −20° C. for a minimum of 20 minutes. The wells were washed with 1×PBS then blocked in 1×PBS/0.1% BSA for 1 h at room temperature. A rabbit anti-Ad antibody (Abcam, Cambridge, MA) was added at 1:8,000 dilution in blocking buffer (0.25 ml per well) and incubated for 1 h at room temperature. The wells were washed 4× with 0.5 mL PBS per well. A HRP conjugated Goat anti-Rabbit antibody (Bethyl Labs, Montgomery Texas) diluted 1000× was added per well and incubated for 1 h prior to a final round of washing. 5 PBS washes were performed and the plates were developed using DAB (Diaminobenzidine tetrahydrochloride) substrate in Tris buffered saline (0.67 mg/mL DAB in 50 mM Tris pH 7.5, 150 mM NaCl) with 0.01% H₂O₂. Wells were developed for 5 min prior to counting. Cells were counted under a 10× objective using a dilution that gave between 4-40 stained cells per field of view. The field of view that was used was a 0.32 mm² grid of which there were equivalent to 625 per field of view on a 24 well plate. The number of infectious viruses/mL was determined by the number of stained cells per grid multiplied by the number of grids per field of view multiplied by a dilution factor 10. Similarly, when working with GFP expressing cells florescent can be used rather than capsid staining to determine the number of GFP expressing virions per mL.

### SAM Vectors

A RNA alphavirus backbone for the antigen expression system was generated from a self-replicating Venezuelan Equine Encephalitis (VEE) virus (Kinney, 1986, Virology 152: 400-413) by deleting the structural proteins of VEE located 3′ of the 26S sub-genomic promoter (VEE sequences 7544 to 11,175 deleted; numbering based on Kinney et al 1986; SEQ ID NO:6). To generate the self-amplifying mRNA (“SAM”) vector, the deleted sequences were replaced by antigen sequences. A representative SAM vector containing 20 model antigens is “VEE-MAG25mer” (SEQ ID NO:4). The vectors featuring the antigen cassettes described having the MAG25mer cassette can be replaced by the antigen cassettes described above, e.g., in Table 2B.

### In Vitro Transcription to Generate SAM

For in vivo studies: SAM vectors were generated as “AU-SAM” vectors. A modified T7 RNA polymerase promoter (TAATACGACTCACTATA), which lacks the canonical 3′ dinucleotide GG, was added to the 5′ end of the SAM vector to generate the in vitro transcription template DNA (SEQ ID NO:10,755; 7544 to 11,175 deleted without an inserted antigen cassette). Reaction conditions are described below:
1× transcription buffer (40 mM Tris-HCL [pH7.9], 10 mM dithiothreitol, 2 mM spermidine, 0.002% Triton X-100, and 27 mM magnesium chloride) using final concentrations of 1×T7 RNA polymerase mix (E2040S); 0.025 mg/mL DNA transcription template (linearized by restriction digest); 8 mM CleanCap Reagent AU (Cat. No. N-7114) and 10 mM each of ATP, cytidine triphosphate (CTP), GTP, and uridine triphosphate (UTP)
Transcription reactions are incubated at 37° C. for 2 hr and treated with final 2 U DNase I (AM2239)/0.001 mg DNA transcription template in DNase I buffer for 1 hr at 37° C.
SAM was purified by RNeasy Maxi (QIAGEN, 75162)

Alternatively to co-transcriptional addition of a 5′ cap structure, a 7-methylguanosine or a related 5′ cap structure can be enzymatically added following transcription using a vaccinia capping system (NEB Cat. No. M2080) containing mRNA 2′-O-methyltransferase and S-Adenosyl methionine.

### Immunizations

For ChAdV68 vaccines with single constructs, transgenic mice expressing a chimeric HLA-A01:01 [Taconic Model #9662-F (CB6F1 background; CB6F1-Tg(HLA-A*0101/H2-Kb)A1.01)] or HLA-A02:01 [Taconic Model #9659-F (CB6F1 background; CB6F1-Tg(HLA-A*0201/H2-Kb)A*0201)] were injected with 5×10¹⁰ viral particles (VP), in 100 μL volume, bilateral intramuscular injection (50 μL per leg).

For bilateral strategies of co-administering CTA and KRAS-encoding ChAdV68 vaccines separately, 5×10¹⁰ viral particles (VP) of each vaccine, in 50 μL volume, was injected in the left Tibialis Anterior (TA) for the CTA-encoding vaccine and the right TA for the KRAS-encoding vaccine.

For “blended” strategies of co-administering separate CTA and KRAS-encoding ChAdV68 vaccines as a single vaccination, 5×10¹⁰ viral particles (VP) of each vaccine, in 100 μL total volume, was mixed and administered as bilateral intramuscular injections (50 μL per leg).

For samRNA vaccines with single constructs, 10 μg of RNA-LNP complexes in 100 μL volume were administered as a bilateral intramuscular injection (50 μL per leg).

For bilateral strategies of co-administering CTA and KRAS-encoding samRNA vaccines separately, 5 μg of each RNA-LNP complex, in 50 μL volume, was injected in the left TA for the CTA-encoding vaccine and the right TA for the KRAS-encoding vaccine.

For “blended” strategies of co-administering separate CTA and KRAS-encoding samRNA vaccines as a single vaccination, 5 μg of each RNA-LNP complex, in 50 μL volume were mixed (total volume of 100 μL) and administered as separate bilateral intramuscular injection in the left TA and the right TA (50 μL per leg).

### Splenocyte Dissociation

Splenocytes were isolated 14 days post-immunization. Spleens for each mouse were pooled in 3 mL of complete RPMI (RPMI, 10% FBS, penicillin/streptomycin). Mechanical dissociation was performed using the gentleMACS Dissociator (Miltenyi Biotec), following manufacturer's protocol. Dissociated cells were filtered through a 40 micron filter and red blood cells were lysed with ACK lysis buffer (150 mM NH₄Cl, 10 mM KHCO₃, 0.1 mM Na₂EDTA). Cells were filtered again through a 30 micron filter and then resuspended in complete RPMI. Cells were counted on the Cytoflex LX (Beckman Coulter) using propidium iodide staining to exclude dead and apoptotic cells. Cell were then adjusted to the appropriate concentration of live cells for subsequent analysis.

### Ex Vivo Enzyme-Linked Immunospot (ELISpot) Analysis

ELISPOT analysis was performed according to ELISPOT harmonization guidelines {DOI: 10.1038/nprot.2015.068} with the mouse IFNg ELISpotPLUS kit (MABTECH). 1×10⁵ splenocytes were incubated with 10 uM of the indicated peptides for 16 hours in 96-well IFNg antibody coated plates. Spots were developed using alkaline phosphatase. The reaction was timed for 10 minutes and was terminated by running plate under tap water. Spots were counted using an AID vSpot Reader Spectrum. For ELISPOT analysis, wells with saturation >50% were recorded as “too numerous to count”. Samples with deviation of replicate wells >10% were excluded from analysis. Spot counts were then corrected for well confluency using the formula: spot count+2×(spot count x % confluence/[100%−% confluence]). Negative background was corrected by subtraction of spot counts in the negative peptide stimulation wells from the antigen stimulated wells. Finally, wells labeled too numerous to count were set to the highest observed corrected value, rounded up to the nearest hundred.

### XVI.C. Identification of Shared Antigens

Shared antigen gene-based targets were identified using three computational steps: First, genes were identified with low or no expression in most normal tissues using data available through the Genotype-Tissue Expression (GTEx) Project [1]. Aggregated gene expression data was obtained from the Genotype-Tissue Expression (GTEx) Project (version V7p2). This dataset comprised greater than 11,000 post-mortem samples from greater than 700 individuals and greater than 50 different tissue types. Expression was measured using RNA-Seq and computationally processed according to the GTEx standard pipeline (https://www.gtexportal.org/home/documentationPage). Gene expression was calculated using the sum of isoform expressions that were calculated using RSEM v 1.2.22 [2].

Next, genes were identified which are aberrantly expressed in cancer samples using data from The Cancer Genome Atlas (TCGA) Research Network: http://cancergenome.nih.gov/. Greater than 11,000 samples available from TCGA (Data Release 6.0) were examined.

Finally, in these genes, peptides were identified which are likely to be presented as cell surface antigens by MHC Class I proteins using a deep learning model trained on HLA presented peptides sequenced by MS/MS, as described in international patent application no. PCT/US2016/067159, herein incorporated by reference, in its entirety, for all purposes.

To identify the common tumor antigens (CTAs; shared antigens), criteria to exclude genes that were expressed in normal tissue that were strict enough to ensure tumor specificity, but would account for potential artifacts such as read misalignment, were defined. Genes were eligible for inclusion as CTAs if they met the following criteria: The median GTEx expression in each organ that was a part of the brain, heart, or lung was less than 0.1 transcripts per million (TPM) with no one sample exceeding 5 TPM. The median GTEx expression in other essential organs was less than 2 TPM with no one sample exceeding 10 TPM. Expression was ignored for organs classified as non-essential (testis, thyroid, and minor salivary gland). Genes were considered expressed in tumor samples if they had expression in TCGA of greater than 10 TPM in at least 30 samples. Based on literature review, we also added the genes MAGEB4 and MAGEB6.

The gene CTAG1A/CTAG1B (NY-ESO-1) was also added. As it's expression is not accurately quantified using the computational methodology in TCGA Data Release 6.0, the RSEM calculations available in the TCGA legacy archive (https://portal.gdc.cancer.gov/legacy-archive) which accounts for multiply mapped reads were relied upon.

The distribution of the expression of the remaining genes across TCGA samples was then examined. When the known CTAs were examined, e.g. the MAGE family of genes, expression of these genes in log space was generally characterized by a bimodal distribution. This distribution included a left mode around a lower expression value and a right mode (or thick tail) at a higher expression level. This expression pattern is consistent with a biological model in which some minimal expression can be detected at baseline in many samples and higher expression of the gene is observed in a subset of tumors experiencing epigenetic dysregulation. The distribution of expression of each gene across TCGA samples was reviewed and those where only a unimodal distribution with no significant right-hand tail was observed were discarded. A small number of genes were eliminated by hand curation, for example, if they were likely to be expressed in a tissue not available in GTEx. This resulted in a set of 59 genes. See Table 3. In Table 3 an X is used to indicate cancers in which the gene is expressed at greater than 10 TPM in at least 1% of cases.

TABLE 3
Analysis of Expression Levels in Cancer Subtypes

| | | | | | | Cervical | | | | | | |
|---|---|---|---|---|---|---|---|---|---|---|---|---|
| | | | | | | Squamous | | | | | | |
| | | | | | | Cell | | | | | Head | |
| | | | Bladder | | | Carcinoma | | | | | and | |
| | Acute | Adreno- | Urothe- | Brain | Breast | And Endo- | | Colon | Esoph- | | Neck | |
| | Myeloid | cortical | lial | Lower | Invasive | cervical | Cholan- | Adeno- | ageal | Glioblas- | Squamous | Kidney |
| | Leuke- | Carci- | Carci- | Grade | Carci- | Adeno- | giocar- | carci- | Carci- | toma | Cell | Chromo- |
| Gene | mia | noma | noma | Glioma | noma | carcinoma | cinoma | noma | noma | Multiforme | Carcinoma | phobe |
| ACTL8 | | X | X | | X | X | | X | X | | X | |
| ADAM2 | | | | | X | | | | | | | X |
| ADAM7 | | | | | | | | | | | | |
| AMELX | | | | | | | | X | | | | |
| BPIFA2 | | X | | | | | | | | | X | X |
| CRYGC | | | | | | | | | | | | |
| CT83 | | X | X | | X | X | X | X | X | | X | |
| CTAG1A/ | | X | X | | X | X | X | X | X | | X | |
| CTAG1B | | | | | | | | | | | | |
| CTCFL | | | | | | X | | | X | | X | |
| DCAF12L1 | | | | | | X | | | | | | |
| DCAF4L2 | | X | X | | | | X | | X | | X | |
| DEFB126 | | X | X | | | | | | X | | | |
| DMRT1 | | | | | | X | X | | | | | |
| DPPA2 | | | X | | | | | | X | | X | |
| FMR1NB | | | | | | | | | X | | | |
| FTHL17 | | | X | | X | X | | | X | | X | |
| GAGE1 | | | X | | | | | | | | | |
| GAGE121 | | | X | | | | | | X | | | |
| GAGE2A | | | X | | | X | | | X | | X | X |
| GLYATL3 | | | | | X | X | | | | | | |
| GPRC6A | | X | | | | | | | | | | X |
| GSG1L2 | | | | | | | | | | | | |
| IFNK | X | | X | | | | | X | | | X | |
| IL22RA2 | X | | | | X | X | | | X | | X | |
| INSL4 | | | | | | X | | X | | | | |
| INSL6 | X | | X | | X | X | | | | | | |
| KCNU1 | | | | | | | | | | | | X |
| LIN28A | | | | | X | | X | | | | | |
| LUZP4 | | | | | | | | | | | | |
| MAGEA1 | | X | X | | X | X | X | X | X | | X | X |
| MAGEA10 | | | X | | X | X | | | X | | X | |
| MAGEA11 | | | X | | | X | | X | X | | X | |
| MAGEA3 | | X | X | | X | X | X | X | X | | X | X |
| MAGEA4 | | | X | | X | X | | X | X | | X | |
| MAGEA6 | | X | X | | X | X | X | X | X | | X | X |
| MAGEA9 | | | X | | | | | | X | | X | |
| MAGEB1 | | | | | | | | | X | | X | |
| MAGEB2 | | X | X | | X | X | | X | X | | X | X |
| MAGEB4 | | | | | X | | | | | | | |
| MAGEB6 | | X | | | | | | | | | | |
| MAGEC1 | | X | X | | X | | | | X | | X | X |
| MAGEC2 | | X | X | | X | X | | | X | X | X | X |
| MORC1 | | X | | | | | | | | | | |
| PAGE1 | | | X | | | X | | | X | | | X |
| PAGE5 | | X | X | | X | | X | | X | | X | X |
| PASD1 | | | X | | | | | | X | | | |
| PRDM7 | | | | | | | | | | | | |
| PROKR1 | | | | | | | | | | | | |
| R3HDML | | | | | | | | X | | | | |
| SLC7A13 | | | | | | | | | | | | X |
| SMC1B | X | | X | | X | X | | | X | | X | X |
| SMR3A | | | | | | | | | | | X | |
| SSX1 | | | X | | X | | X | | X | | | X |
| STRA8 | | | X | | X | X | | | X | | X | |
| TFDP3 | | | | | X | | | | | | | |
| XAGE3 | | | X | | | | | | | | | |
| XAGE5 | | | | | | | | | | | | |
| ZFP42 | | | X | | X | X | X | | X | | X | |
| ZNF560 | X | X | X | X | | | | | | X | | |

| | | Kidney | | | | | | | | | |
|---|---|---|---|---|---|---|---|---|---|---|---|
| | Kidney | Renal | | | Lung | Lymphoid | | | | | |
| | Renal | Papil- | Liver | | Squa- | Neoplasm | | | | Pheochro- | |
| | Clear | lary | Hepato- | Lung | mous | Diffuse | | Ovarian | Pancre- | mocytoma | Prostate |
| | Cell | Cell | cellular | Adeno- | Cell | Large | | Serous | atic | and | Adeno- |
| | Carci- | Carci- | Carci- | car- | Carci- | B-cell | Mesothe- | Cystadeno- | Adenocar- | Paragan- | carci- |
| Gene | noma | noma | noma | cinoma | noma | Lymphoma | lioma | carcinoma | cinoma | glioma | noma |
| ACTL8 | | | X | X | X | X | X | X | X | X | |
| ADAM2 | | | | | | | | | | | X |
| ADAM7 | | X | | | | | | | | | X |
| AMELX | | | | | | | | | | | |
| BPIFA2 | | | X | X | X | | X | | X | | |
| CRYGC | | | | | | | | X | | | |
| CT83 | | X | X | X | X | | | X | X | | |
| CTAG1A/ | | X | X | X | X | X | X | X | | X | X |
| CTAG1B | | | | | | | | | | | |
| CTCFL | | | | | X | X | | X | | | |
| DCAF12L1 | X | X | | | | | | X | | | |
| DCAF4L2 | | | X | X | X | | | | | | |
| DEFB126 | | | | | | | | X | | | |
| DMRT1 | | | | | X | X | | X | | | |
| DPPA2 | | X | | X | X | | | X | | | |
| FMR1NB | | | | X | | | | | | | |
| FTHL17 | | | X | X | X | | | | | | |
| GAGE1 | | | X | | X | | | X | | | |
| GAGE121 | | | | | X | | | X | | | |
| GAGE2A | | X | X | X | X | | X | X | | | |
| GLYATL3 | | | | | X | | | | X | | X |
| GPRC6A | X | | | | | | | | | | |
| GSG1L2 | X | | | | | | | | | | |
| IFNK | | | | X | | | | | | | |
| IL22RA2 | | | | X | X | X | | | | | |
| INSL4 | | | | X | X | | | | X | | |
| INSL6 | | | X | X | | | | X | | | |
| KCNU1 | | | X | | | | | | | | |
| LIN28A | | | | | | | | | | | |
| LUZP4 | | | | | | | | | | | |
| MAGEA1 | | | X | X | X | X | X | X | X | X | |
| MAGEA10 | | | | X | X | | | X | | | |
| MAGEA11 | | | X | X | X | | | X | | | |
| MAGEA3 | | X | X | X | X | | X | X | X | X | X |
| MAGEA4 | | | X | X | X | X | | X | | | |
| MAGEA6 | | X | X | X | X | | X | X | X | X | |
| MAGEA9 | | | | | X | X | | | | | |
| MAGEB1 | | | X | X | X | X | | | | | |
| MAGEB2 | X | | X | X | X | X | X | X | | | |
| MAGEB4 | | | | | | | | | | | |
| MAGEB6 | | | | | | | | | | | |
| MAGEC1 | | | X | X | X | X | | X | | | |
| MAGEC2 | X | X | X | X | X | | | X | | X | X |
| MORC1 | | | | | | | | | | | |
| PAGE1 | | X | X | X | X | | X | X | | | |
| PAGE5 | | X | X | X | X | X | | X | | X | |
| PASD1 | | | | X | X | | X | X | | | |
| PRDM7 | | | | | | | | | | | |
| PROKR1 | | | | | | | | | | X | |
| R3HDML | | | | | | | | | | | |
| SLC7A13 | X | X | | | | | | | | | |
| SMC1B | | | X | X | X | X | | X | | | |
| SMR3A | | | X | | | | | | | | |
| SSX1 | | | X | X | X | | | X | | | |
| STRA8 | X | X | | X | X | | | X | | | |
| TFDP3 | | | X | | | | | | | | |
| XAGE3 | | | | | X | | | | | | |
| XAGE5 | | | X | | X | | | | | | |
| ZFP42 | | | | X | X | | X | X | | | |
| ZNF560 | | | | X | X | | X | X | | X | X |

| | | | | | | | | Uterine | | |
|---|---|---|---|---|---|---|---|---|---|---|
| | | | Skin | | | | | Corpus | | |
| | Rectum | | Cutane- | | | | | Endome- | | |
| | Adeno- | | ous | Stomach | | Thyroid | Uterine | trial | Uveal | Total |
| | carci- | Sarco- | Mela- | Adenocar- | Thymo- | Carci- | Carcino- | Carci- | Mela- | Indi- |
| Gene | noma | ma | noma | cinoma | ma | noma | sarcoma | noma | noma | cations |
| ACTL8 | X | X | X | X | | | X | X | X | 22 |
| ADAM2 | | | | | | | | | | 3 |
| ADAM7 | | | | | | | | | | 2 |
| AMELX | X | X | | | | | X | | | 4 |
| BPIFA2 | | | | X | | | X | | | 10 |
| CRYGC | | | | | | | | X | | 2 |
| CT83 | X | | X | X | | | X | X | | 19 |
| CTAG1A/ | X | X | X | X | | | X | X | X | 24 |
| CTAG1B | | | | | | | | | | |
| CTCFL | | | X | X | | | X | X | | 10 |
| DCAF12L1 | | X | X | | | | X | X | | 8 |
| DCAF4L2 | | | | X | | | X | | | 10 |
| DEFB126 | | | | | | | | X | | 6 |
| DMRT1 | | | | | | | X | X | | 7 |
| DPPA2 | | | | | | | | X | | 8 |
| FMR1NB | | | | | | | X | | | 3 |
| FTHL17 | | | | | | | | | | 8 |
| GAGE1 | | X | X | X | | | X | | | 8 |
| GAGE121 | | | X | X | | | | | | 6 |
| GAGE2A | | X | X | X | | | X | X | | 16 |
| GLYATL3 | | | | | | | X | X | | 7 |
| GPRC6A | | | | | | | | | | 3 |
| GSG1L2 | | | | | | | | | | 1 |
| IFNK | | | | | | | | | | 5 |
| IL22RA2 | | | | | | | | | | 8 |
| INSL4 | | | | | | | X | X | | 7 |
| INSL6 | | | | X | | | X | X | | 10 |
| KCNU1 | | | X | | | | | | | 3 |
| LIN28A | | | X | | | | X | X | | 5 |
| LUZP4 | | | X | | | | X | | | 2 |
| MAGEA1 | X | X | X | X | X | | X | | | 24 |
| MAGEA10 | X | X | X | X | | | X | X | | 14 |
| MAGEA11 | X | X | X | X | | | X | X | | 15 |
| MAGEA3 | X | X | X | X | | | X | X | | 24 |
| MAGEA4 | X | X | X | X | X | | X | X | | 18 |
| MAGEA6 | X | X | X | X | | | X | X | | 23 |
| MAGEA9 | | | | | | | X | | | 6 |
| MAGEB1 | | X | X | X | | | X | | | 10 |
| MAGEB2 | X | X | X | X | | | X | X | | 21 |
| MAGEB4 | | | | | | | | | | 1 |
| MAGEB6 | | | | | | | | | | 1 |
| MAGEC1 | X | X | X | X | | | X | X | | 17 |
| MAGEC2 | X | X | X | X | | | X | X | | 22 |
| MORC1 | | | X | | | | | | | 2 |
| PAGE1 | X | X | X | X | | | X | X | | 16 |
| PAGE5 | | X | X | X | | | X | X | | 19 |
| PASD1 | | | X | X | | | X | X | | 10 |
| PRDM7 | | | | | | | | | | 1 |
| PROKR1 | | | | | | | X | | | 2 |
| R3HDML | X | | | | | | | | | 2 |
| SLC7A13 | | | | | | | | | | 3 |
| SMC1B | | X | X | | | | | X | | 15 |
| SMR3A | | | | | | X | | X | | 4 |
| SSX1 | | X | X | X | | X | X | | X | 15 |
| STRA8 | | | | X | | | | X | | 12 |
| TFDP3 | | | | | X | | | | | 3 |
| XAGE3 | | X | X | | | | X | X | | 7 |
| XAGE5 | | X | X | | | | X | | | 5 |
| ZFP42 | | | X | X | | | X | X | | 14 |
| ZNF560 | | X | X | | X | X | X | | X | 17 |

To identify peptides that are likely to be presented as cell surface antigens by MHC Class I proteins, a sliding window was used to parse each of these proteins into its constituent 8-11 amino acid sequences. These peptides and their flanking sequences were processed with the HLA peptide presentation deep learning model to calculate the probability of presentation of each peptide at the 99.9^{th} percentile expression level observed for this gene in TCGA. A peptide was considered likely to be presented (i.e., a candidate target) if its quantile normalized probability of presentation calculated by the model was greater than 0.001.

To prioritize genes that are likely to be relevant to a given indication, genes where the gene is expressed at a level of at least 10 TPM in at least 0.98% of cancer cases were selected.

A total of 10698 shared antigen sequences were identified. Refer to SEQ ID NOS. 57-10,754 found in International Application publication WO2019226941A1, which is herein incorporated by references for all purposes. Predicted shared antigens associated with gene expressed at a level of at least 10 TPM in at least 0.98% of cancer cases. Each of the above sequence identifiers is associated with the gene name, amino acid sequence of the peptide, Ensembl ID, and corresponding HLA allele(s).

Cancer Testis Antigen (CTA) epitopes that have been validated as presented by specific HLAs (data not shown) were assessed for estimated prevalence across various populations for lung adenocarcinoma (LUAD), lung squamous cell carcinoma (LUSC), ovarian serous cystadenocarcinoma (HGSOC), and head and neck squamous cell carcinoma (HNSC). Results are shown in Table 4.

TABLE 4
CTA Prevalence

| | | | | | | | Total # | |
|---|---|---|---|---|---|---|---|---|
| | | | | | | | tumors | Tumors |
| Gene | HLA | Peptide | LUAD | LUSC | HGSOC | HNSC | targeted | detected |
| MAGEA1 | HLA-A*02:01 | 9mer | 3.06 | 12.07 | 2.28 | 5.38 | 95 | 7 |
| MAGEA1 | HLA-A*25:01 | 11mer | 0.22 | 0.86 | 0.16 | 0.38 | 19 | 2 |
| MAGEA1 | HLA-A*26:01 | 11mer | 0.42 | 1.65 | 0.31 | 0.74 | 17 | 2 |
| MAGEA1 | HLA-A*33:03 | 9mer | 0.24 | 0.95 | 0.18 | 0.42 | 3 | 1 |
| MAGEA1 | HLA-B*35:01 | 9mer | 0.78 | 3.08 | 0.58 | 1.37 | 17 | 4 |
| MAGEA1 | HLA-B*44:02 | 9mer | 0.82 | 3.25 | 0.61 | 1.45 | 19 | 1 |
| MAGEA1 | HLA-B*44:02 | 11mer | 0.82 | 3.25 | 0.61 | 1.45 | 20 | 1 |
| MAGEA1 | HLA-C*02:02 | 9mer | 0.57 | 2.24 | 0.42 | 1.00 | 25 | 3 |
| MAGEA1 | HLA-C*12:03 | 9mer | 0.60 | 2.38 | 0.45 | 1.06 | 39 | 6 |
| MAGEA11 | HLA-A*01:01 | 9mer | 0.50 | 7.82 | 3.92 | 5.13 | 59 | 11 |
| MAGEA11 | HLA-A*02:01 | 9mer | 0.87 | 13.65 | 6.84 | 8.94 | 66 | 4 |
| MAGEA11 | HLA-B*15:01 | 9mer | 0.18 | 2.89 | 1.45 | 1.89 | 16 | 1 |
| MAGEA3 | HLA-A*01:01 | 9mer | 4.46 | 9.76 | 0.52 | 8.21 | 64 | 13 |
| MAGEA3 | HLA-A*29:02 | 9mer | 1.37 | 2.99 | 0.16 | 2.52 | 6 | 1 |
| MAGEA3 | HLA-B*15:01 | 9mer | 1.65 | 3.60 | 0.19 | 3.03 | 19 | 3 |
| MAGEA4 | HLA-A*01:01 | 9mer | 0.38 | 11.51 | 5.62 | 8.03 | 35 | 1 |
| MAGEA4 | HLA-A*02:01 | 9mer-1 | 3.06 | 20.08 | 9.81 | 14.01 | 116 | 5 |
| MAGEA4 | HLA-A*02:01 | 10mer | 3.06 | 20.08 | 9.81 | 14.01 | 121 | 24 |
| MAGEA4 | HLA-A*02:01 | 9mer-2 | 3.06 | 20.08 | 9.81 | 14.01 | 40 | 4 |
| MAGEA4 | HLA-A*02:05 | 10mer | 0.18 | 1.21 | 0.59 | 0.84 | 3 | 1 |
| MAGEA4 | HLA-B*15:01 | 11mer | 0.65 | 4.25 | 2.08 | 2.96 | 22 | 1 |
| MAGEA4 | HLA-B*35:01 | 9mer | 0.78 | 5.12 | 2.50 | 3.58 | 31 | 5 |
| MAGEA4 | HLA-B*40:01 | 9mer | 0.58 | 3.81 | 1.86 | 2.66 | 8 | 2 |
| MAGEA4 | HLA-B*40:02 | 9mer | 0.20 | 3.81 | 0.66 | 2.66 | 8 | 1 |
| MAGEA4 | HLA-B*41:02 | 9mer | 0.06 | 1.35 | 0.18 | 0.94 | 5 | 1 |
| MAGEA4 | HLA-B*44:02 | 9mer | 0.82 | 5.40 | 2.64 | 3.77 | 33 | 5 |
| MAGEA4 | HLA-B*44:05 | 9mer | 0.03 | 0.17 | 0.08 | 0.12 | 3 | 2 |
| MAGEA4 | HLA-C*15:02 | 9mer | 0.26 | 1.74 | 0.85 | 1.21 | 4 | 1 |
| MAGEA6 | HLA-A*01:01 | 9mer | 4.05 | 9.67 | 0.33 | 7.99 | 20 | 2 |
| MAGEA6 | HLA-B*18:01 | 10mer | 1.33 | 3.17 | 0.11 | 2.62 | 14 | 1 |
| MAGEA6 | HLA-B*35:01 | 9mer | 1.80 | 4.30 | 0.15 | 3.56 | 14 | 1 |
| MAGEA6 | HLA-B*44:03 | 10mer | 1.75 | 4.18 | 0.14 | 3.46 | 8 | 1 |
| MAGEA8 | HLA-A*02:01 | 9mer | 1.31 | 0.55 | 0.80 | 0.40 | 37 | 1 |
| MAGEA9 | HLA-A*01:01 | 9mer | 0 | 0.80 | 0.20 | 0.36 | 9 | 1 |
| MAGEA9 | HLA-A*03:02 | 9mer | 0 | 0.02 | 0 | 0.01 | 2 | 1 |
| MAGEA9 | HLA-A*11:01 | 9mer | 0 | 0.37 | 0.09 | 0.17 | 10 | 1 |
| MAGEA9 | HLA-A*68:01 | 9mer | 0 | 0.14 | 0.03 | 0.06 | 2 | 1 |
| MAGEA9 | HLA-C*12:03 | 9mer | 0 | 0.28 | 0.07 | 0.12 | 16 | 2 |
| MAGEB2 | HLA-A*02:01 | 10mer | 2.33 | 8.78 | 0.68 | 4.99 | 104 | 21 |
| MAGEC2 | HLA-A*25:01 | 9mer | 0.27 | 0.31 | 0.22 | 0.19 | 2 | 2 |
| CT83 | HLA-A*01:01 | 9mer | 9.05 | 5.49 | 0.26 | 0.77 | 69 | 16 |
| CT83 | HLA-A*02:01 | 9mer | 15.79 | 9.57 | 0.46 | 1.35 | 102 | 3 |
| CT83 | HLA-B*07:02 | 9mer | 7.45 | 4.51 | 0.22 | 0.63 | 18 | 1 |
| CTCFL | HLA-B*07:02 | 9mer | 0.17 | 1.33 | 7.21 | 0.56 | 5 | 2 |
| CTCFL | HLA-B*40:02 | 10mer | 0.02 | 0.19 | 1.02 | 0.08 | 5 | 1 |
| DCAF12L1 | HLA-A*01:01 | 9mer | 0.13 | 0.13 | 2.09 | 0 | 25 | 1 |
| DCAF12L1 | HLA-A*02:01 | 9mer | 0.22 | 0.24 | 3.65 | 0 | 4 | 1 |
| SMC1B | HLA-A*24:02 | 9mer | 0.70 | 0.43 | 0.57 | 1.82 | 28 | 3 |
| ZNF560 | HLA-A*01:01 | 9mer | 0.96 | 0.40 | 1.18 | 0.09 | 50 | 4 |

Prevalence >0.5% in bold

### XVI.D. CTA Epitope Vaccine Cassette Design

CTA epitopes were analyzed for inclusion in a vaccine cassette. HLA/Peptide targets were chosen based on prevalence in population and mass-spectrometry confirmation. All tumors targeted with the indicated HLA/peptide were included in the analysis count. Low confidence confirmed samples were counted as validated. When a sample was found to have >1 HLA/peptide found in the proposed cassette version confirmed by mass spectrometry with a heavy standard, it was counted as validated.

Results for selected CTA epitopes presented by HLA A*01:01 (Table 5A) are shown in for the various indicated tumors (Table 5B).

TABLE 5A
HLA A*01:01 Cassette Epitopes

| Gene | Peptide |
|---|---|
| MAGEA3/A6 | FVQENYLEY |
| MAGEA11 | EVDPTSHSY |
| CT83 | NTDNNLAVY |
| MAGEA3 | EVDPIGHLY |

TABLE 5B
HLA A*01:01 Cassette Analysis

| | # Total | # Validated | % Validated |
|---|---|---|---|
| ALL TUMORS | 86 | 33 | 38 |
| Lung | 42 | 16 | 38 |
| Breast | 1 | 0 | 0 |
| Gastric | 1 | 0 | 0 |
| Stomach | 2 | 0 | 0 |
| Colon | 20 | 12 | 60 |
| Ovary | 6 | 0 | 0 |
| Head & Neck | 14 | 2 | 14 |

Results for selected CTA epitopes presented by HLA A*02:01 (Table 6A) are shown in for the various indicated tumors (Table 6B).

TABLE 6A
HLA A*02:01 Cassette Epitopes

| Gene | Peptide |
|---|---|
| MAGEA4 | GVYDGREHTV |
| MAGEA11 | ALREEGEGV |
| MAGEA1 | KVLEYVIKV |
| MAGEB2 | GVYDGEEHSV |
| CT83 | KLVELEHTL |

TABLE 6B
HLA A*02:01 Cassette Analysis

| | # Total | # Validated | % Validated |
|---|---|---|---|
| ALL TUMORS | 171 | 42 | 25 |
| Breast | 2 | 0 | 0 |
| Colon | 25 | 4 | 16 |
| Esophagus | 1 | 0 | 0 |
| Gastric | 2 | 1 | 50 |
| Head & Neck | 31 | 11 | 35 |
| Lung | 74 | 16 | 22 |
| Lymphoma | 2 | 0 | 0 |
| Ovary | 19 | 7 | 37 |
| Pancreas | 6 | 0 | 0 |
| Stomach | 3 | 1 | 33 |
| Bladder | 4 | 1 | 25 |
| Cervix | 1 | 1 | 100 |
| Kidney | 1 | 0 | 0 |

Note:
Count reflects tumors targeted for the CTA A2 peptides

Results for selected CTA epitopes presented by HLA A*01:01 or HLA A*02:01 (Table 7A) are shown in for the various indicated tumors (Table 71B).

TABLE 7A
HLA A*01:01/A*02:01 Cassette Epitopes

| Gene | Allele | Peptide |
|---|---|---|
| MAGEA11 | A*01:01 | EVDPTSHSY |
| CT83 | A*01:01 | NTDNNLAVY |
| MAGEA3 | A*01:01 | EVDPIGHLY |
| MAGEA4 | A*02:01 | GVYDGREHTV |
| MAGEB2 | A*02:01 | GVYDGEEHSV |
| CT83 | A*02:01 | KLVELEHTL |

TABLE 7B
HLA A*01:01/A*02:01 Cassette Analysis

| | # Total | # Validated | % Validated |
|---|---|---|---|
| ALL TUMORS | 231 | 67* | 29 |
| Bladder | 4 | 1 | 25 |
| Breast | 3 | 0 | 0 |
| Cervix | 1 | 1 | 100 |
| Colon | 36 | 13 | 36 |
| Esophagus | 1 | 0 | 0 |
| Gastric | 2 | 1 | 50 |
| Head & Neck | 38 | 13 | 34 |
| Kidney | 1 | 0 | 0 |
| Lung | 108 | 30 | 28 |
| Lymphoma | 2 | 0 | 0 |
| Ovary | 25 | 7 | 28 |
| Pancreas | 6 | 0 | 0 |
| Stomach | 4 | 1 | 25 |

*19 of these tumors have >1 validation

Results for selected CTA epitopes for a first “Pan CTA” cassette (Table 8A) are shown in for the various indicated tumors (Table 81B). Prevalence of expression of HLA/gene combination related to this cassette design was assessed (Table 8C).

TABLE 8A
Pan CTA Cassette Epitopes

| Gene | Allele | Peptide |
|---|---|---|
| MAGEA1/A4 | B*44:02, B*44:05, | AETSYVKVL, |
| | B*40:01, B*40:02, | EYVIKVSAR* |
| | B*41:02, A*33:03* | |
| MAGEA4 | B*35:01 | SALPTTISF |
| MAGEA4 | A*02:01, A*02:05, | GVYDGREHTVY, |
| | B*15:01 | GVYDGREHTV |
| MAGEB2 | A*02:01 | GVYDGEEHSV |
| MAGEA11 | A*01:01 | EVDPTSHSY |
| CT83 | A*01:01, B*07:02* | NTDNNLAVY, |
| | | LVRPSSSGL* |
| MAGEA3 | A*01:01 | EVDPIGHLY |

TABLE 8B
Pan CTA Cassette Analysis

| | Total | Validated | % Validated |
|---|---|---|---|
| ALL TUMORS | 250 | 78 | 31 |
| Bladder | 7 | 3 | 43 |
| Breast | 2 | 0 | 0 |
| Cervix | 1 | 1 | 100 |
| Colon | 36 | 14 | 39 |
| Esophagus | 3 | 0 | 0 |
| Gastric | 2 | 2 | 100 |
| Head & Neck | 46 | 17 | 37 |
| Kidney | 1 | 0 | 0 |
| Lung | 111 | 31 | 28 |
| Lung adeno | 52 | 14 | 27 |
| Lung Squam | 42 | 13 | 31 |
| Lymphoma | 4 | 0 | 0 |
| Ovary | 31 | 9 | 29 |
| Pancreas | 3 | 0 | 0 |
| Stomach | 3 | 1 | 33 |

TABLE 8C
Pan CTA Cassette - Prevalence of expression
of HLA/gene combination

| Tissue | Total | TPM ≥1 for PanCTA | % Prevalence |
|---|---|---|---|
| All Tumors | 545 | 300* | 55 |
| All Lung | 179 | 130 | 73 |
| Lung squam | 64 | 52 | 81 |
| Lung adeno | 97 | 65 | 67 |
| CRC | 129 | 50 | 39 |
| Ovary | 87 | 49 | 56 |
| Head & Neck | 53 | 37 | 70 |
| lymph nodes | 1 | 1 | 100 |
| Pancreas | 23 | 4 | 17 |
| Kidney | 15 | 1 | 7 |
| Breast | 10 | 4 | 40 |
| Bladder | 10 | 9 | 90 |
| Cervix | 9 | 2 | 22 |
| Glioblastoma | 9 | 0 | 0 |
| Stomach | 7 | 3 | 43 |
| Lymphoma | 4 | 4 | 100 |
| Gastric | 4 | 2 | 50 |
| Esophagus | 3 | 3 | 100 |
| maxillary sinus | 1 | 1 | 100 |
| Gastric/Esophageal | 1 | 0 | 0 |

*185 tumors have >1 HLA/gene combo included in pan-slate

Results for selected CTA epitopes for a second “Pan CTA ALT” cassette (Table 9A) are shown in for the various indicated tumors (Table 91B).

TABLE 9A
Pan CTA ALT Cassette Epitopes

| Gene | Allele | Peptide |
|---|---|---|
| MAGEA1/ | B*44:02, B*44:05, | AETSYVKVL, |
| A4 | B*40:01, B*40:02, | EYVIKVSAR* |
| | B*41:02, A*33:03* | |
| MAGEA4 | B*35:01 | SALPTTISF |
| MAGEA4 | A*02:01, A*02:05, | GVYDGREHTVY, |
| | B*15:01 | GVYDGREHTV |
| CT83 | A*02:01 | KLVELEHTL |
| CTCFL | B*07:02 | GPRQSLQQC |
| CT83 | A*01:01, B*07:02*, | NTDNNLAVY, |
| | B*40:01* | LVRPSSSGL*, |
| | | GEMSSNSTAL* |
| MAGEA3 | A*01:01 | EVDPIGHLY |

TABLE 9B
Pan CTA ALT Cassette Analysis

| | Total | Validated | % Validated |
|---|---|---|---|
| ALL TUMORS | 262 | 62 | 24 |
| Bladder | 4 | 0 | 0 |
| Breast | 4 | 0 | 0 |
| Cervix | 2 | 1 | 50 |
| Colon | 38 | 13 | 34 |
| Esophagus | 3 | 0 | 0 |
| Gastric | 1 | 0 | 0 |
| Head & Neck | 43 | 13 | 30 |
| Kidney | 1 | 0 | 0 |
| Lung | 124 | 27 | 22 |
| Lung adeno | 65 | 13 | 20 |
| Lung Squam | 52 | 13 | 25 |
| Lymphoma | 3 | 0 | 0 |
| Ovary | 29 | 8 | 28 |
| Pancreas | 7 | 0 | 0 |
| Stomach | 3 | 0 | 0 |

TABLE 9C
Pan CTA ALT Cassette - Prevalence of
expression of HLA/gene combination

| | | TPM ≥1 for | Pan | Original |
|---|---|---|---|---|
| Tissue | Total | PanCTA | CTA ALT | Pan CTA |
| All Tumors | 545 | 329 | 60 | 55 |
| All Lung | 179 | 138 | 77 | 73 |
| Lung squam | 64 | 53 | 83 | 81 |
| Lung adeno | 97 | 71 | 73 | 67 |
| CRC | 129 | 51 | 40 | 39 |
| Ovary | 87 | 61 | 70 | 56 |
| Head & Neck | 53 | 38 | 72 | 70 |
| lymph nodes | 1 | 1 | 100 | 100 |
| Pancreas | 23 | 9 | 39 | 17 |
| Kidney | 15 | 1 | 7 | 7 |
| Breast | 10 | 6 | 60 | 40 |
| Bladder | 10 | 8 | 80 | 90 |
| Cervix | 9 | 4 | 44 | 22 |
| Glioblastoma | 9 | 0 | 0 | 0 |
| Stomach | 7 | 3 | 43 | 43 |
| Lymphoma | 4 | 4 | 100 | 100 |
| Gastric | 4 | 1 | 25 | 50 |
| Esophagus | 3 | 3 | 100 | 100 |
| maxillary sinus | 1 | 1 | 100 | 100 |
| Gastric/Esophageal | 1 | 0 | 0 | 0 |

Results for selected CTA epitopes for a “HLA B-Allele” cassette (Table 10A) are shown in for the various indicated tumors (Table 10B).

TABLE 10A
HLA B-Allele Cassette Epitopes

| Gene | Allele | Peptide |
|---|---|---|
| MAGEA1/ | B*44:02, B*44:05, | AETSYVKVL |
| A4 | B*40:01, B*40:02, | |
| | B*41:02 | |
| MAGEA1 | B*35:01 | EADPTGHSY |
| MAGEA4 | B*35:01 | SALPTTISF |
| MAGEA4 | B*15:01 | GVYDGREHTVY |
| MAGEA3 | B*15:01 | TQHFVQENY |

TABLE 10B
HLA B-Allele Cassette Analysis

| | Total | Validated | % Validated |
|---|---|---|---|
| ALL TUMORS | 106 | 23 | 22 |
| Bladder | 2 | 0 | 0 |
| Cervix | 1 | 0 | 0 |
| Colon | 12 | 1 | 8 |
| Esophagus | 2 | 0 | 0 |
| Gastric | 1 | 0 | 0 |
| Head & Neck | 22 | 11 | 50 |
| Lung | 49 | 9 | 18 |
| Lymphoma | 2 | 0 | 0 |
| Ovary | 13 | 2 | 15 |
| Pancreas | 2 | 0 | 0 |

A summary of the cassette analysis is shown in Table 11A and Table 11B.

The PAN CTA cassette demonstrated the best combined validation data and TPM prevalence for Lung and Ovarian cancers and was the best across multiple indications. Multiple targets were present in greater than a third of tumors and covered a large population.

The PAN CTA ALT cassette demonstrated the best TPM prevalence for multiple indications, with Ovarian improved relative to the first original PAN CTA cassette. Prevalence was higher in multiple indications based on TPM data and TPM data suggests multiple targets were present in greater than a third of tumors.

The A*01:01 CTA cassette demonstrated the best validation data for Colon and Lung cancers.

The B-Allele CTA cassette demonstrated the best validation for Head & Neck cancer.

TABLE 11A
Cassette Validation Summary

| | ALL | | | | Head |
|---|---|---|---|---|---|
| | TUMORS | COLON | LUNG | OVARY | &Neck |
| CTA | 38% | 60% | 38% | 0% | 14% |
| A*01:01 | (33/86) | (12/20) | (16/42) | (0/6) | (2/14) |
| CTA | 25% | 16% | 22% | 37% | 35% |
| A*02:01 | (42/171) | (4/25) | (16/74) | (7/19) | (11/31) |
| CTA | 29% | 36% | 28% | 28% | 34% |
| A1/A2 | (67/231)* | (13/36) | (30/108) | (7/25) | (13/38) |
| CTA | 22% | 8% | 18% | 15% | 50% |
| B-allele | (23/106) | (1/12) | (9/49) | (2/13) | (11/22) |
| PAN | 31% | 39% | 28% | 29% | 37% |
| CTA | (78/250)** | (14/36) | (31/111) | (9/31) | (17/46) |
| PAN | 24% | 34% | 22% | 28% | 30% |
| CTA ALT | (62/262)*** | (13/38) | (27/124) | (8/29) | (13/43) |

*19 out of 67 have >1 Validated Target (28%)
**30 out of 78 have >1 Validated Target (38%)
***17 out of 62 have >1 Validated Target (27%)

TABLE 11B
Cassette Prevalence of Expression Summary (TPM ≥ 1)

| | | PAN_CTA | Original |
|---|---|---|---|
| Tissue | Total | ALT** | PAN CTA |
| All Tumors | 545 | 60 | 55 |
| All Lung | 179 | 77 | 73 |
| Lung squam | 64 | 83 | 81 |
| Lung adeno | 97 | 73 | 67 |
| CRC | 129 | 40 | 39 |
| Ovary | 87 | 70 | 56 |
| Head & Neck | 53 | 72 | 70 |
| lymph nodes | 1 | 100 | 100 |
| Pancreas | 23 | 39 | 17 |
| Kidney | 15 | 7 | 7 |
| Breast | 10 | 60 | 40 |
| Bladder | 10 | 80 | 90 |
| Cervix | 9 | 44 | 22 |
| Glioblastoma | 9 | 0 | 0 |
| Stomach | 7 | 43 | 43 |
| Lymphoma | 4 | 100 | 100 |
| Gastric | 4 | 25 | 50 |
| Esophagus | 3 | 100 | 100 |
| maxillary sinus | 1 | 100 | 100 |
| Gastric/Esophageal | 1 | 0 | 0 |

*185 tumors have >1 HLA/gene combo included in pan-slate
**195 tumors have >1 HLA/gene combo in pan slate ALT

Prevalence analyses on additional CTA cassette designs which include KRAS G12V and KRAS G12C epitopes along with CTA epitopes (Tables 12A and 12B). Prevalence of expression for the HLA/gene and/or HLA/gene/mutation combination related to the CTA/neo population focused cassette design in various lung cancers was assessed. (Table 13A).

From the analysis of 179 lung tumors, 84% had multiple epitopes restricted on the highly shared HLAs shown in Table 12B, which match the population focused design, and 60% of these have ≥5 epitopes.

TABLE 12A
Pan CTA + KRAS Cassette Epitopes

| Gene | Allele | Peptide |
|---|---|---|
| MAGEA1/A8 | A*02:01*, A*02:05*, | KVLEYVIKV*, |
| | B*44:02, B*44:05, B*40:01, | AETSYVKVL, |
| | B*40:02, B*41:02, | EYVIKVSAR** |
| | A*33:03 ** | |
| MAGEA4 | B*35:01 | SALPTTISF |
| MAGEA4/A8 | A*02:01*, C*15:02*, | KVLEHVVRV*, |
| | B*44:02, B*44:05, B*40:01, | AETSYVKVL, |
| | B*40:02, B*41:02 | |
| MAGEA4 | A*02:01, A*02:05, B*15:01 | GVYDGREHTVY, |
| | | GVYDGREHTV |
| MAGEB2 | A*02:01 | GVYDGEEHSV |
| MAGEA11 | A*01:01 | EVDPTSHSY |
| CT83 | A*01:01, B*07:02*, | NTDNNLAVY, |
| | B*40:01** | LVRPSSSGL*, |
| | | GEMSSNSTAL** |
| MAGEA3 | A*01:01 | EVDPIGHLY |
| KRAS-G12C | A*02:01*, A*03:01, | KLVVVGACGV*, |
| | A*11:01, A*68:01, | VVVGACGVGK, |
| | B*07:02**, C*03:04** | GACGVGKSAL** |
| KRAS-G12V | A*02:01*, A*03:01, | KLVVVGAVGV*, |
| | A*11:01, A*68:01, | VVVGAVGVGK, |
| | B*07:02**, C*01:02**, | AVGVGKSAL**, |
| | C*03:04*** | GAVGVGKSAL*** |

Presentation of above HLA-restricted peptides has been validated in tumors (Table 9B) and tumor cell lines.

TABLE 12B
Population coverage-focused CTA/neo
Cassette Epitopes

| Gene | Allele | Peptide |
|---|---|---|
| MAGEA4 | A*02:01 | GVYDGREHTV, |
| | A*03:01, A*11:01 | TVYGEPRKL |
| MAGEA4, | A*03:01, A*11:01 | ALAETSYVK* |
| MAGEA8 | B*07:02 | TSYVKVLEH |
| | A*02:01 | YPSLREAAL* |
| | | KVLEHVVRV |
| | | ALLEEEEGV |
| CT83 | A*02:01 | KLVELEHTL |
| CT83 | A*01:01 | NTDNNLAVY |
| | B*07:02 | LVRPSSSGL |
| | B*40:01 | GEMSSNSTAL |
| MAGEA3 | A*01:01 | EVDPIGHLY |
| MAGEA6 | A*01:01 | FVQENYLEY |
| CTCFL | B*07:02 | GPRQSLQQC |
| KRAS_G12C | A*02:01 | KLVVVGACGV |
| | A*03:01, A*11:01 | VVVGACGVGK |
| | B*07:02 | GACGVGKSAL |
| KRAS_G12V | A*02:01 | KLVVVGAVGV |
| | A*03:01, A*11:01 | VVVGAVGVGK |
| | B*07:02 | AVGVGKSAL |

TABLE 13A
Pan CTA ALT Cassette - Prevalence of
expression of HLA/gene combination

| | | HLA- |
|---|---|---|
| | | filtered |
| | Prevalence | Prevalence* |
| Indication | (%) | (%) |
| All Lung | 74 | 81 |
| Lung squam | 72 | N/A |
| Lung adeno | 75 | N/A |

*Note:
HLA-filtered is a subpopulation requiring A*03:01, A*11:01 or A*02:01 to be included

Prevalence analyses on additional CTA cassette designs combined with a KRAS G12V, KRAS G12D, KRAS G12C and KRAS Q61H epitopes in a CTA+KRAS vaccine was completed (Tables 14A, 14B, and 14C). Prevalence of expression for the HLA/gene and/or HLA/gene/mutation combination related to the CTA/KRAS population focused vaccine design in lung and colorectal cancers was assessed. (Table 15).

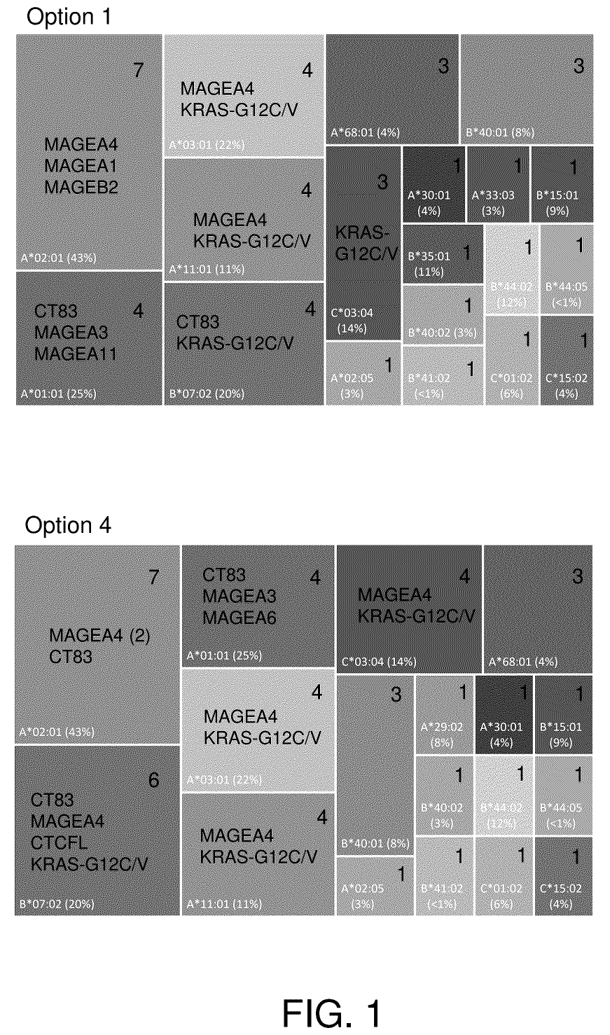

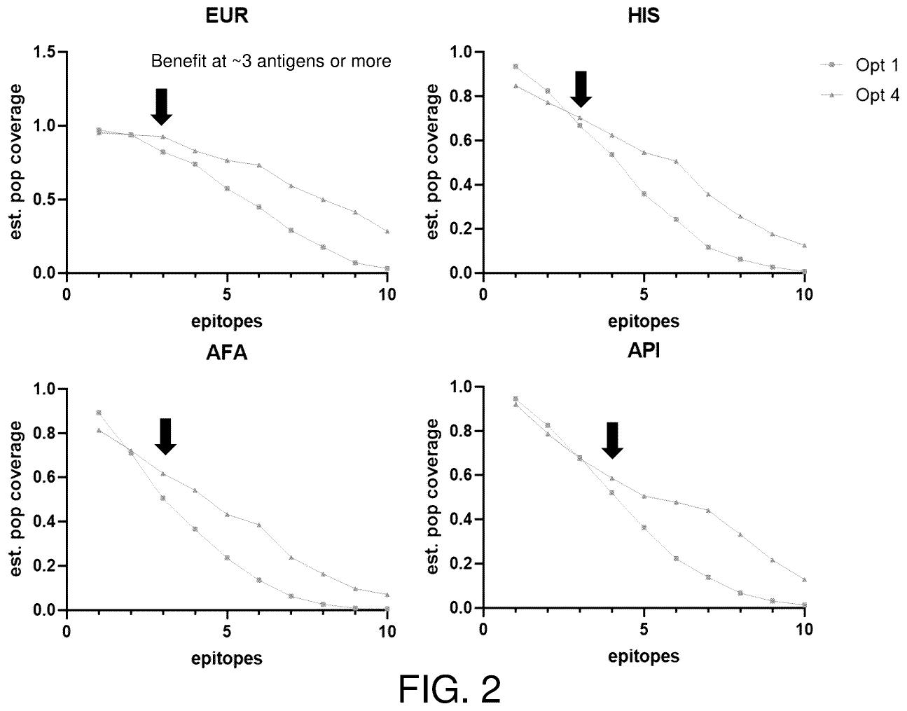

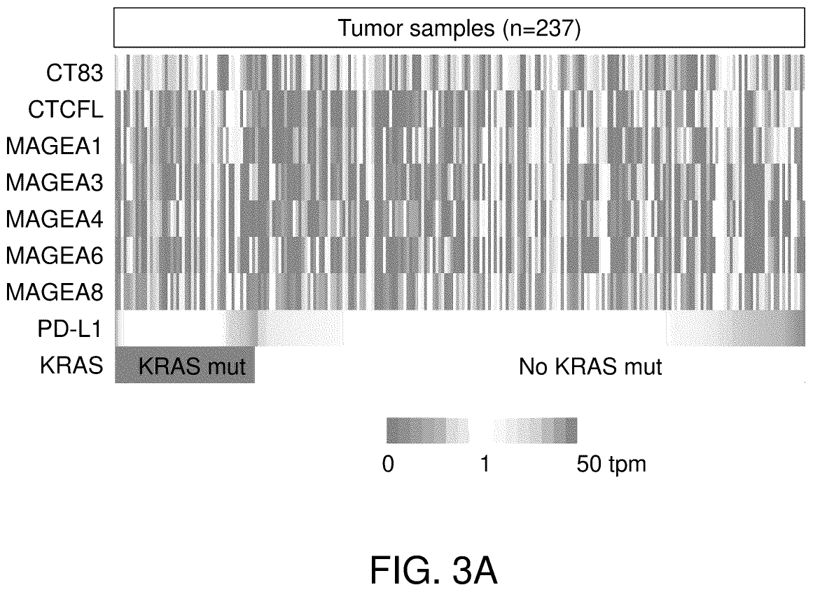

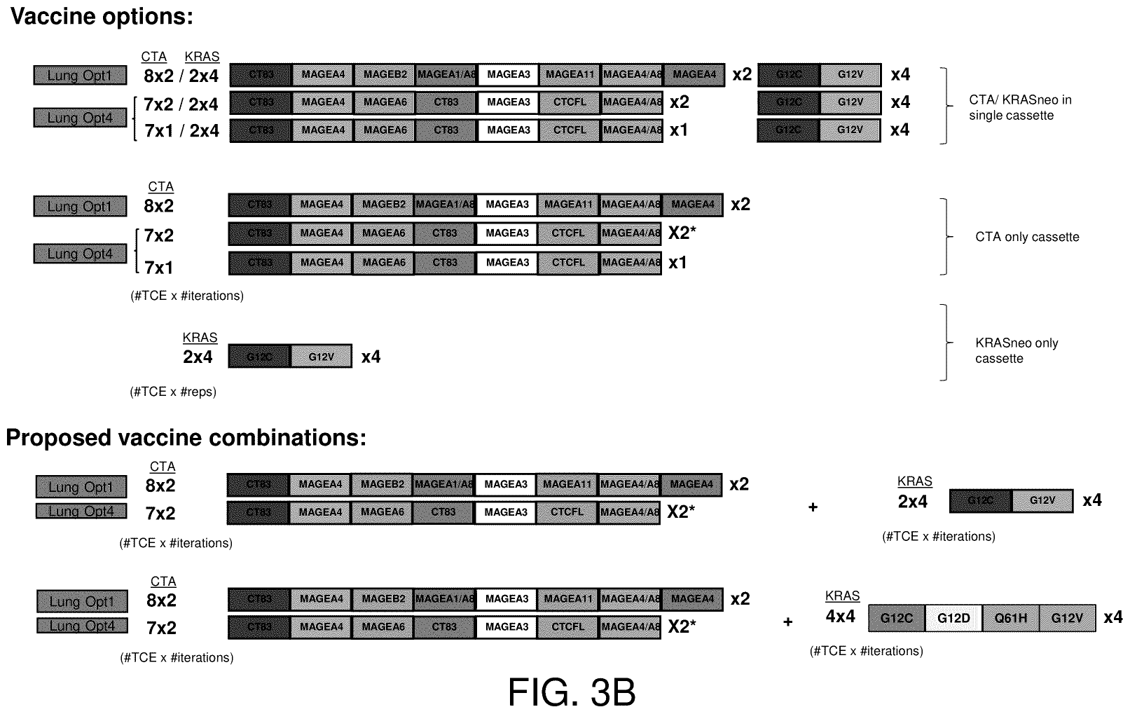

From the analysis of 237 lung tumors, 71% have at least 1 epitope restricted on the highly shared HLAs shown in Table 15, which match the Option 1 design when combined with KRAS vector; 74% of these have ≥2 epitopes, while 57% have ≥3 epitopes. From the same analysis of lung tumors, 71% have at least 1 epitope restricted on the highly shared HLAs shown in Table 15, which match the Option 4 design when combined with KRAS vector; 85% of these have ≥2 epitopes, while 67% have ≥3 epitopes. From the analysis of 213 colorectal tumors, 52% have at least 1 epitope restricted on the highly shared HLAs shown in Table 15, which match the Option 1 design when combined with KRAS vector; 73% of these have ≥2 epitopes, while 55% have ≥3 epitopes. From the same analysis of CRC tumors, 52% have at least 1 epitope restricted on the highly shared HLAs shown in Table 15, which match the Option 4 design when combined with KRAS vector; 75% of these have ≥2 epitopes, while 60% have ≥3 epitopes. FIG. 1 provides a graphical illustration of the HLA coverage, shown as the number of TCE epitopes validated in tumors by mass spectrometry in each HLA group, (% US population with at least 1 allele shown in parentheses). FIG. 2 shows population coverage for the Option 4 CTA cassette across various groups. FIG. 3A illustrates that CTA and KRAS mutations have heterogenous co-expression in low PD-L1 lung tumors, indicating a higher likelihood for multiple antigen targets in vaccine strategies including both CTA and KRAS epitopes.

TABLE 14A
CTA Option 1 (8x2) Cassette Epitopes

| Gene | Allele | Peptide |
|---|---|---|
| CT83 | A*01:01 | NTDNNLAVY |
| CT83 | B*07:02 | LVRPSSSGL |
| CT83 | B*40:01 | GEMSSNSTAL |
| MAGEA4 | A*02:01, A*02:05 | GVYDGREHTV |
| MAGEA4 | B*15:01 | GVYDGREHTVY |
| MAGEA4/A1/A8 | A*11:01 | ALAETSYVK |
| MAGEA4/A8 | A*02:01; C*15:02 | KVLEHVVRV |
| MAGEA4 | B*35:01 | SALPTTISF |
| MAGEB2 | A*02:01 | GVYDGEEHSV |
| MAGEA1/8 | A*02:01 | KVLEYVIKV |
| MAGEA1/A4 | A*30:01, B*44:02, | AETSYVKVL |
| | B*44:05, | |
| | B*40:01, B*40:02, | |
| | B*41:02 | |
| MAGEA1 | A*33:03 | EYVIKVSAR |
| MAGEA3 | A*01:01 | EVDPIGHLY |
| MAGEA3 | B*40:01 | MEVDPIGHL |
| MAGEA11 | A*01:01 | EVDPTSHSY |

Presentation of above HLA-restricted peptides has been validated in tumors (Table 9B) and tumor cell lines.

TABLE 14B
CTA Option 4 (8x2) Cassette Epitopes

| Gene | Allele | Peptide |
|---|---|---|
| CT83 | A*01:01 | NTDNNLAVY |
| CT83 | B*07:02 | LVRPSSSGL |
| CT83 | B*40:01 | GEMSSNSTAL |
| CT83 | A*02:01 | KLVELEHTL |
| MAGEA4 | A*02:01, A*02:05 | GVYDGREHTV |
| MAGEA4 | B*15:01 | GVYDGREHTVY |
| MAGEA4/A1/A8 | A*11:01 | ALAETSYVK |
| MAGEA4/A8 | A*02:01; C*15:02 | KVLEHVVRV |
| MAGEA4 | A*02:01 | ALLEEEEGV |
| MAGEA4 | B*07:02 | YPSLREAAL |
| MAGEA4 | C*03:04 | IAYPSLREAAL |
| MAGEA1/A4 | A*30:01, B*44:02, | AETSYVKVL |
| | B*44:05, | |
| | B*40:01, B*40:02, | |
| | B*41:02 | |
| MAGEA6/A3 | A*01:01, A*29:02 | FVQENYLEY |
| MAGEA3 | A*01:01 | EVDPIGHLY |
| MAGEA3 | B*40:01 | MEVDPIGHL |
| CTCFL | B*07:02 | GPRQSLQQC |

Presentation of above HLA-restricted peptides has been validated in tumors (Table 9B) and tumor cell lines.

TABLE 14C
KRAS Cassette Epitopes

| Gene | Allele | Peptide |
|---|---|---|
| KRAS_G12C | A*02:01 | KLVVVGACGV |
| KRAS_G12C | A*03:01, A*11:01, | VVVGACGVGK |
| | A*68:01 | |
| KRAS_G12C | B*07:02, C*03:04 | GACGVGKSAL |
| KRAS_G12V | A*02:01 | KLVVVGAVGV |
| KRAS_G12V | A*03:01, A*11:01, | VVVGAVGVGK |
| | A*68:01 | |
| KRAS_G12V | B*07:02, C*01:02 | AVGVGKSAL |
| KRAS_G12V | C*03:04 | GAVGVGKSAL |

TABLE 15
CTA Option 1 and Option 4 Cassettes Combined with KRAS-4 ×
4 Cassette - Prevalence of expression of HLA/gene combination

| | | Population | | |
|---|---|---|---|---|
| | | Prevalence | All samples | # Samples |
| | | (HLA match, | with genes (mut | with ≥3 |
| | | mut or CTA) | or CTA) | epitopes |
| Lung | Opt 1 | 70.9% | 87.3% | 96 |
| | Opt 4 | 70.5% | 87.3% | 111 |
| Colorectal | Opt 1 | 51.6% | 68.1% | 60 |
| | Opt 4 | 51.6% | 71.4% | 66 |

Prevalence analysis performed using sequencing data from internal lung tumor set (N = 237) and CRC set (N = 213); CTA expression = transcripts per million >1

XVI.E. CTA and/or KRAS Epitope Vaccine Evaluation

Vaccine efficacy with cassettes having single or iterated (repeated) CTA epitopes are assessed.

Mice engineered to express human HLA-A01:01 or HLA-A02:01 are immunized with either 5×10¹⁰ VP using a ChAdV68 delivery vector or 10 μg of a SAM delivery vector encoding the cassettes described above and T cell response in splenocytes 2 weeks post immunization is assessed by IFNγ ELISpot following ex vivo stimulation with the relevant CTA peptides. The results demonstrate the CTA vaccines constructed elicit robust immune responses. Additional CTAneo cassettes will be assessed for in vivo immunogenicity in HLA transgenic mice using IFNγ ELISpot.

Monoallelic K562 cell lines engineered to express HLA-A*01:01, HLA-A*02:01 or HLA-A*11:01 and CTA cassettes with single or iterated (repeated) CTA epitopes and/or KRAS neoepitopes are assessed for peptide presentation by targeted mass spectrometry. The results demonstrate the CTA vaccines constructed present HLA-restricted peptides.

### XVI.F. CTA and KRAS Epitope Vaccine Evaluation

Vaccine efficacy with cassettes having single or iterated (repeated) CTA epitopes was assessed, with or without vaccination with KRAS epitopes, including cassettes having iterated KRAS epitopes. Various proposed constructs and vaccines combinations are shown in FIG. 3B.

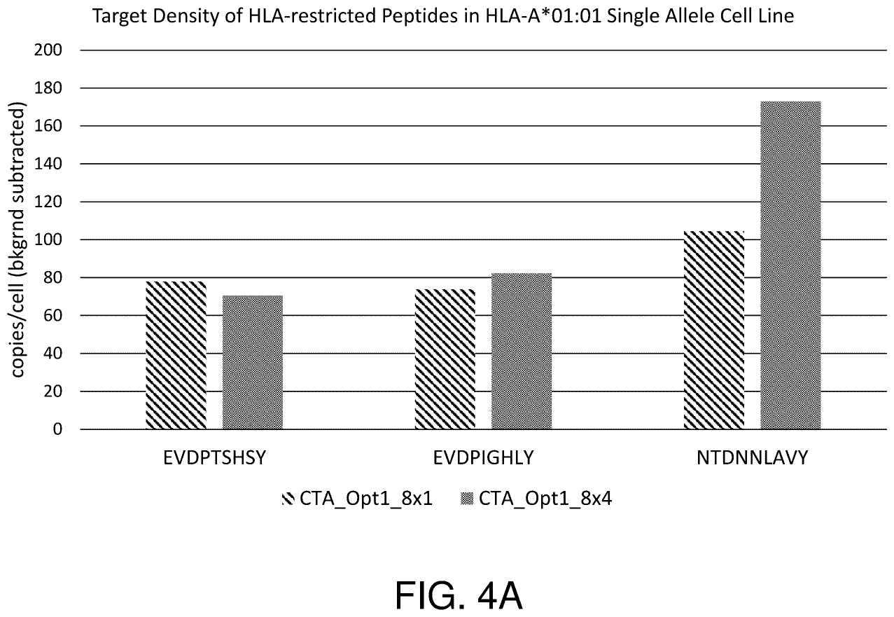

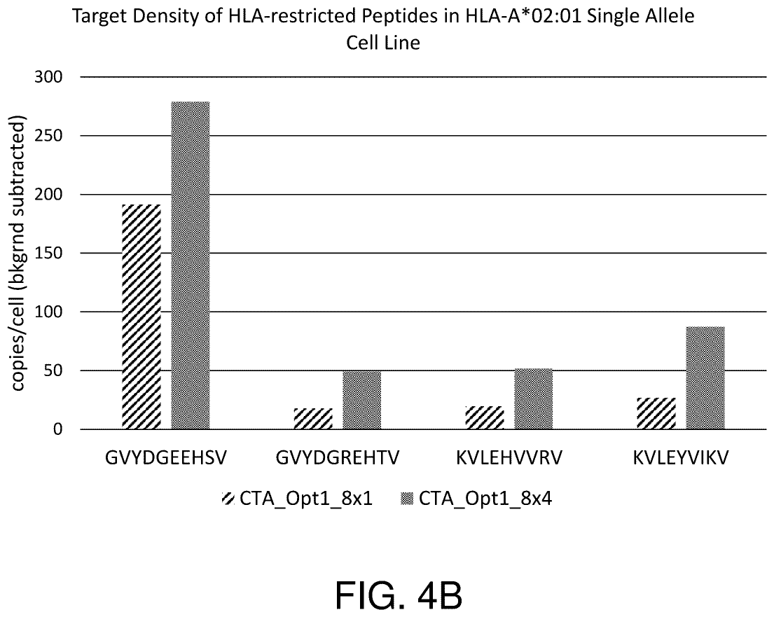

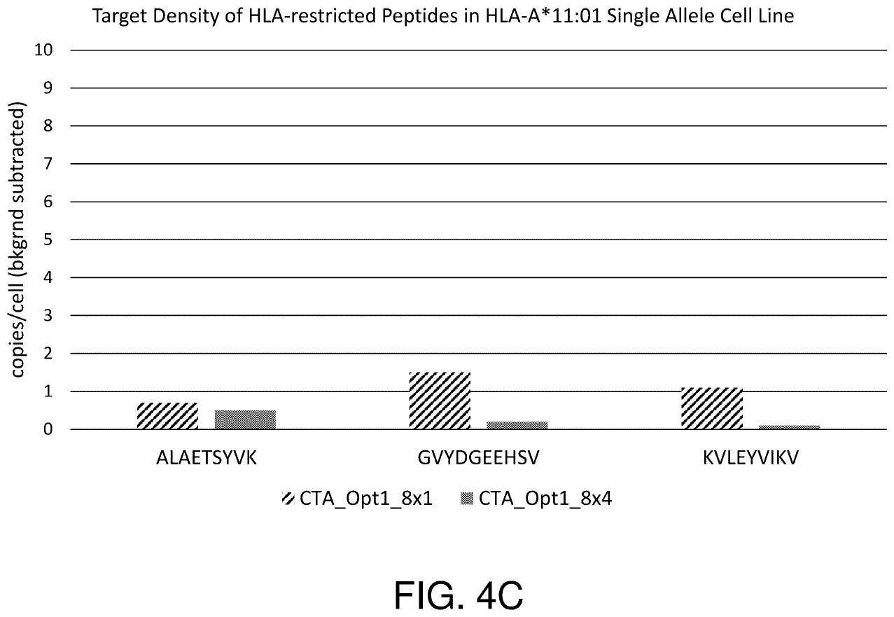

Monoallelic K562 cell lines engineered to express HLA-A*01:01, HLA-A*02:01, or HLA-A*11:01 and CTA cassettes with single (“CTA Opt 1 8×1”) or iterated (repeated; “CTA Opt 1 8×4”) CTA epitopes were assessed for peptide presentation by targeted mass spectrometry, as described herein. Target density for the indicated CTA epitopes encoded by the various CTA cassettes are shown for HLA-A*01:01, HLA-A*02:01, or HLA-A*11:01 in FIG. 4A, FIG. 4B, and FIG. 4C, respectively. For HLA-A*01:01 and HLA-A*02:01, the iterated “8×4” cassettes trended towards increased peptide presentation. For HLA-A*11:01, the cassette expression (qPCR) was observed to be ˜3× lower in the “8×4” compared to the “8×1” cell line, likely driving the apparent decrease in target presentation and the effect of repeated epitope frames in the cassette for HLA-A*11:01 is therefore inconclusive in this cell line. The results demonstrated the CTA vaccines constructed led to presentation of encoded HLA-restricted CTA epitopes, with iterated epitopes generally leading to increased presentation by this assay.

Mice were engineered to express human HLA-A02:01 or HLA-A11:01. The engineered or wild-type B6 mice were immunized as described above using a ChAdV68 delivery vector or a SAM delivery vector encoding the indicated cassettes encoding the sequences described in Table 2B. T cell responses in splenocytes 2 weeks post immunization were assessed by IFNγ ELISpot following ex vivo stimulation with the indicated CTA, KRAS, and/or PADRE peptides.

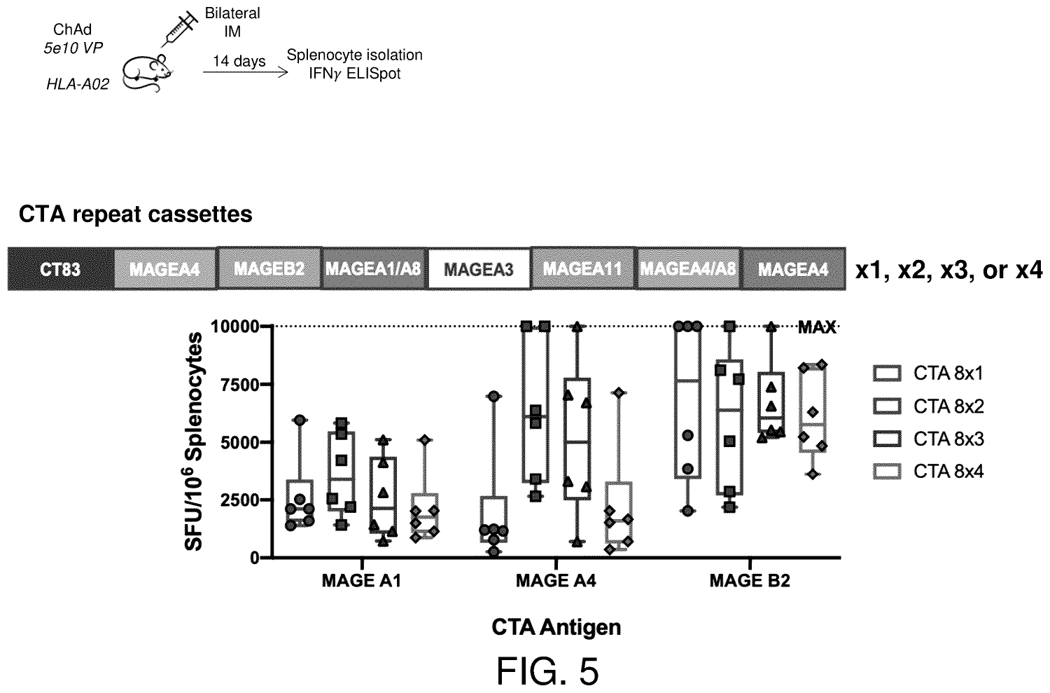

FIG. 5 shows results for HLA-A02:01 expressing mice immunized with ChAdV68 delivery vectors encoding CTA-encoding cassettes having various number of iterations of each CTA epitope, ranging from one to four iterations. The results show the CTA-encoding cassettes with different iterations each demonstrated immunogenicity for MAGE A1, MAGEA4, and MAGEB2 epitopes.

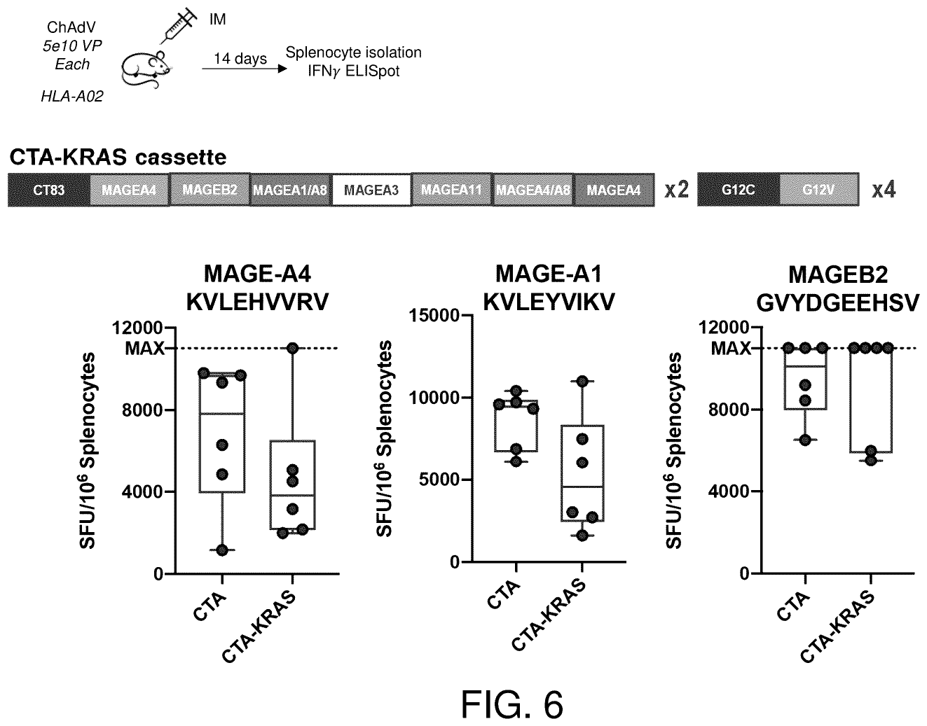

FIG. 6 shows results for HLA-A02:01 expressing mice immunized with ChAdV68 delivery vectors encoding (A) CTA 8×2 “Option 1” (column 1 “CTA”) or (B) CTA 8×2 “Option 1” and KRAS G12V/G12C “2×4” expressed as a single polypeptide with KRAS epitopes interspersed with CTA epitopes. (column 2 “CTA-KRAS”) and stimulated with the indicated predicted MAGE epitopes. The results demonstrate that the CTA-epitope encoding cassettes induced strong T cell responses to MAGE epitopes, with slight decrease with addition of the KRAS cassette. Further constructs with CTA “Option 4” with or without the KRAS G12V & G12C “2×4” cassette were assessed and demonstrated strong T cell responses to MAGE epitopes (data not shown). No KRAS neoepitope responses were observed in HLA-A02:01 or HLA-A11:01 expressing mice (data not shown). PADRE responses were also assessed and were comparable across all constructs (data not shown). No responses were detected in any B6 control mice for any antigen, indicating all responses were driven by the HLA-A02:01 allele (data not shown).

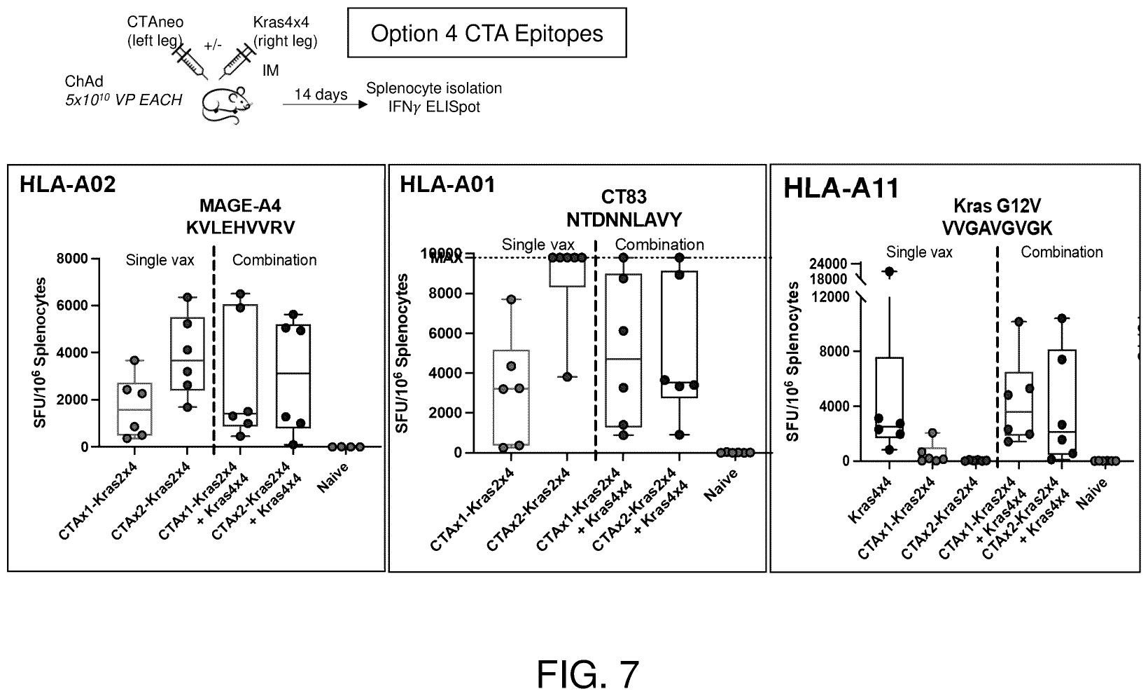

FIG. 7 shows results for HLA-A02:01 and HLA-A01:01 expressing mice immunized with ChAdV68 delivery vectors encoding single iterations of CTA “Option 4” linked to KRAS G12V & G12C “2×4” (column 1), two iterations of CTA “Option 4” linked to KRAS G12V/G12C “2×4” (column 2), single iterations of CTA “Option 4” linked to KRAS G12V & G12C “2×4” administered bilaterally in combination with KRAS “4×4” (column 3), or CTA 8×2 “Option 4” linked to KRAS G12V/G12C “2×4” administered bilaterally in combination with KRAS “4×4” (column 4), and stimulated with the indicated predicted MAGE or CT83 epitopes (left and middle panels, respectively). The results demonstrate that bilateral administration of the separate vectors induced T cell responses to MAGE and CT83 epitopes and the responses were not negatively impacted by the separate administration of the KRAS “4×4” delivery vector. PADRE responses were also assessed and were comparable across all constructs (data not shown). Additional MAGE-A4, MAGE-A3 were also assessed with similar results (data not shown). HLA-A11:01 expressing mice were also immunized with the same constructs (FIG. 7, right panel). The results demonstrate that bilateral administration of the separate vectors induced T cell responses to KRAS neoepitopes and KRAS responses were not negatively impacted by the separate administration of the “CTAneo” cassette delivery vector. Cassettes featuring Option 1 were assessed independently and demonstrated equally potent CTA responses (data not shown).

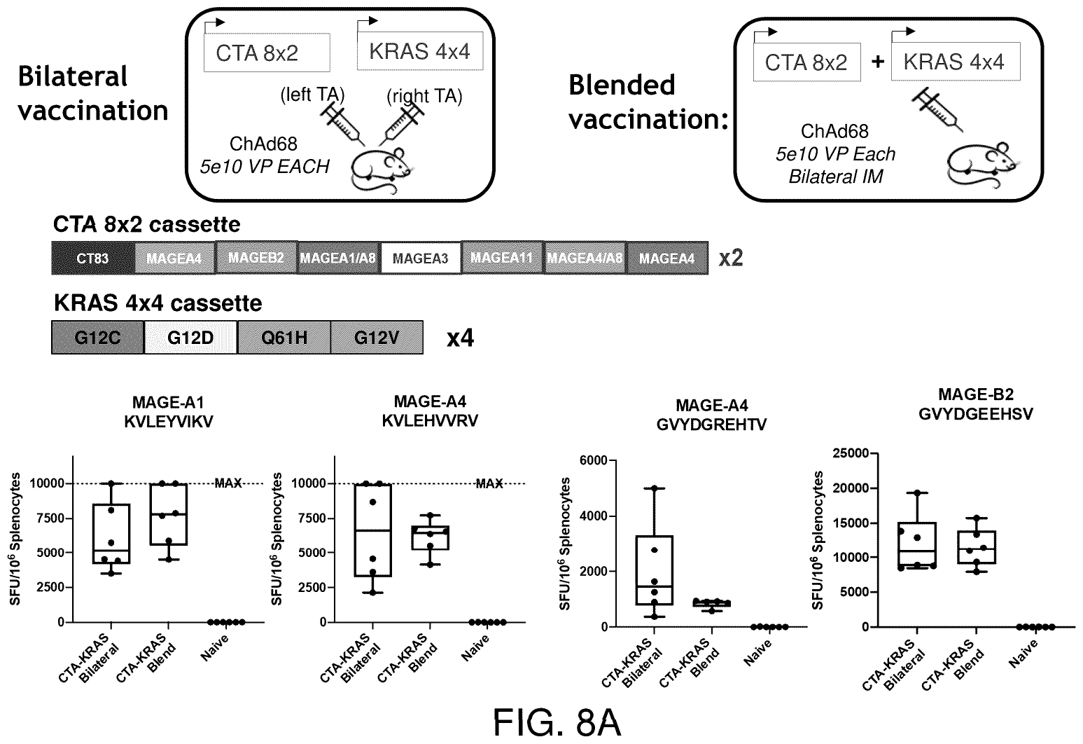

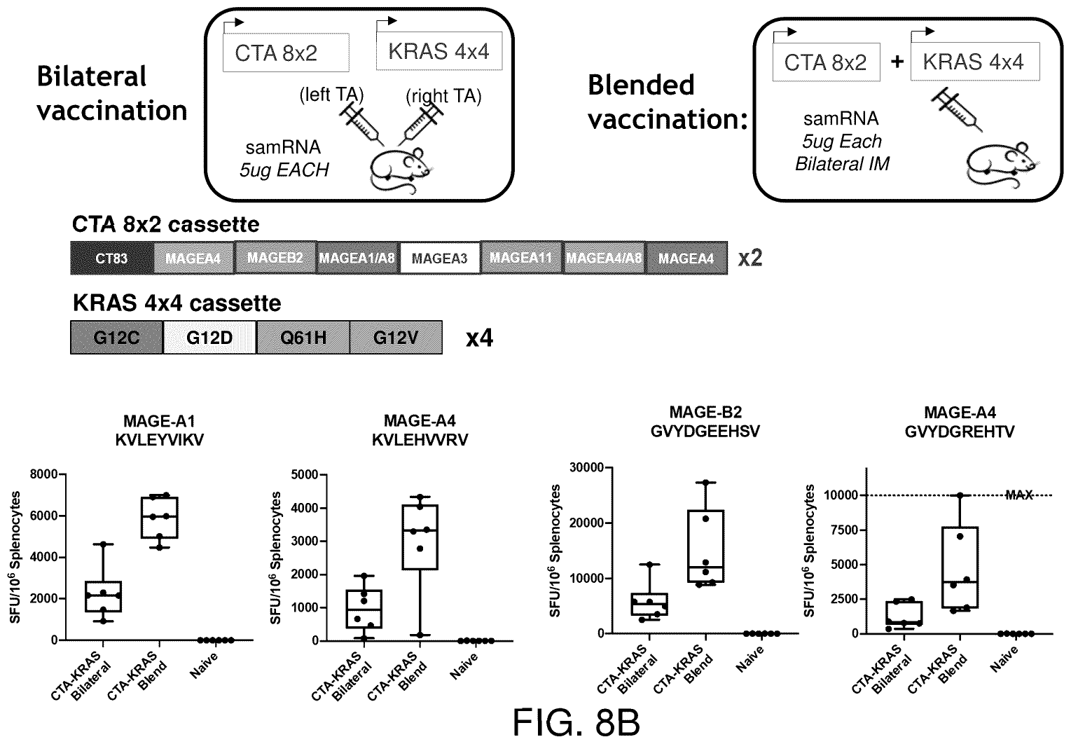

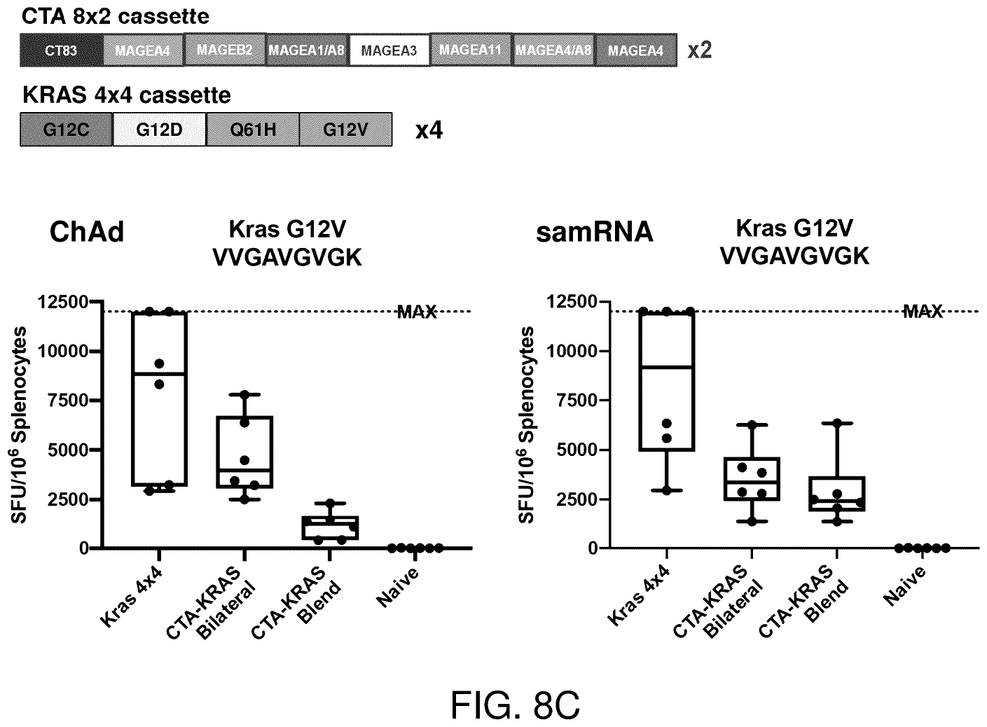

Different administration strategies to deliver CTA and KRAS epitope-encoding vaccines were further assessed. Each of the ChAdV and/or samRNA delivery vectors are administered as (1) a “blended” vaccine strategy administering co-formulated delivery vectors encoding the CTA epitopes alone and the KRAS neoepitopes alone, or (2) as a “bilateral” vaccine strategy separately co-administering a delivery vector encoding the CTA epitopes alone and co-administering a delivery vector encoding the KRAS neoepitopes alone at separate bilateral injection sites. As shown in FIG. 8A, mice administered the bilateral or blended vaccines that included that 8×2 “Option 1” vaccines and KRAS 4×4 vaccines via ChAdV68 delivery vectors demonstrated comparable CTA responses for each administration strategy for the various MAGE epitopes assessed. As shown in FIG. 8B, mice administered the bilateral or blended vaccines that included that 8×2 “Option 1” vaccines and KRAS 4×4 vaccines via samRNA vaccines also demonstrated CTA responses for each administration strategy for the various MAGE epitopes assessed with noticeably stronger responses for the blended strategy. As shown in FIG. 8C, mice administered the bilateral or blended vaccines that included that 8×2 “Option 1” vaccines and KRAS 4×4 vaccines via either ChAdV68 delivery vectors (left panel) or samRNA vaccines (right panel) elicited G12V T cell responses, though lower compared to KRAS 4×4 alone, for both bilateral or blended strategies, with bilateral better in ChAd vaccines and comparable between bilateral or blended strategies for samRNA vaccines.

### Certain Sequences

Vectors, cassettes, and antibodies referred to herein are described below and referred to by SEQ ID NO.

Tremelimumab VL (SEQ ID NO: 16)
Tremelimumab VH (SEQ ID NO: 17)
Tremelimumab VH CDR1 (SEQ ID NO: 18)
Tremelimumab VH CDR2 (SEQ ID NO: 19)
Tremelimumab VH CDR3 (SEQ ID NO: 20)
Tremelimumab VL CDR1 (SEQ ID NO: 21)
Tremelimumab VL CDR2 (SEQ ID NO: 22)
Tremelimumab VL CDR3 (SEQ ID NO: 23)
Durvalumab (MEDI4736) VL (SEQ ID NO: 24)
MEDI4736 VH (SEQ ID NO: 25)
MEDI4736 VH CDR1 (SEQ ID NO: 26)
MEDI4736 VH CDR2 (SEQ ID NO: 27)
MEDI4736 VH CDR3 (SEQ ID NO: 28)
MEDI4736 VL CDR1 (SEQ ID NO: 29)
MEDI4736 VL CDR2 (SEQ ID NO: 30)
MEDI4736 VL CDR3 (SEQ ID NO: 31)
UbA76-25merPDTT nucleotide (SEQ ID NO: 32)
UbA76-25merPDTT polypeptide (SEQ ID NO: 33)
MAG-25merPDTT nucleotide (SEQ ID NO: 34)
MAG-25merPDTT polypeptide (SEQ ID NO: 35)
Ub7625merPDTT_NoSFL nucleotide (SEQ ID NO: 36)
Ub7625merPDTT_NoSFL polypeptide (SEQ ID NO: 37)
ChAdV68.5WTnt.MAG25mer (SEQ ID NO: 2); AC_000011.1 with E1 (nt 577 to 3403) and E3 (nt 27,
125-31, 825) sequences deleted; corresponding ATCC VR-594 nucleotides substituted at five
positions; model neoantigen cassette under the control of the CMV promoter/enhancer inserted in
place of deleted E1; SV40 polyA 3′ of cassette
Venezuelan equine encephalitis virus [VEE] (SEQ ID NO: 3) GenBank: L01442.2
VEE-MAG25mer (SEQ ID NO: 4); contains MAG-25merPDTT nucleotide (bases 30-1755)
Venezuelan equine encephalitis virus strain TC-83 [TC-83] (SEQ ID NO: 5) GenBank: L01443.1
ATAGGCGGCGCATGAGAGAAGCCCAGACCAATTACCTACCCAAAATGGAGAAAGTTCACGTTGACA
TCGAGGAAGACAGCCCATTCCTCAGAGCTTTGCAGCGGAGCTTCCCGCAGTTTGAGGTAGAAGCCA
AGCAGGTCACTGATAATGACCATGCTAATGCCAGAGCGTTTTCGCATCTGGCTTCAAAACTGATCGA
AACGGAGGTGGACCCATCCGACACGATCCTTGACATTGGAAGTGCGCCCGCCCGCAGAATGTATTC
TAAGCACAAGTATCATTGTATCTGTCCGATGAGATGTGCGGAAGATCCGGACAGATTGTATAAGTAT
GCAACTAAGCTGAAGAAAAACTGTAAGGAAATAACTGATAAGGAATTGGACAAGAAAATGAAGGA
GCTCGCCGCCGTCATGAGCGACCCTGACCTGGAAACTGAGACTATGTGCCTCCACGACGACGAGTC
GTGTCGCTACGAAGGGCAAGTCGCTGTTTACCAGGATGTATACGCGGTTGACGGACCGACAAGTCT
CTATCACCAAGCCAATAAGGGAGTTAGAGTCGCCTACTGGATAGGCTTTGACACCACCCCTTTTATG
TTTAAGAACTTGGCTGGAGCATATCCATCATACTCTACCAACTGGGCCGACGAAACCGTGTTAACGG
CTCGTAACATAGGCCTATGCAGCTCTGACGTTATGGAGCGGTCACGTAGAGGGATGTCCATTCTTAG
AAAGAAGTATTTGAAACCATCCAACAATGTTCTATTCTCTGTTGGCTCGACCATCTACCACGAGAAG
AGGGACTTACTGAGGAGCTGGCACCTGCCGTCTGTATTTCACTTACGTGGCAAGCAAAATTACACAT
GTCGGTGTGAGACTATAGTTAGTTGCGACGGGTACGTCGTTAAAAGAATAGCTATCAGTCCAGGCCT
GTATGGGAAGCCTTCAGGCTATGCTGCTACGATGCACCGCGAGGGATTCTTGTGCTGCAAAGTGACA
GACACATTGAACGGGGAGAGGGTCTCTTTTCCCGTGTGCACGTATGTGCCAGCTACATTGTGTGACC
AAATGACTGGCATACTGGCAACAGATGTCAGTGCGGACGACGCGCAAAAACTGCTGGTTGGGCTCA
ACCAGCGTATAGTCGTCAACGGTCGCACCCAGAGAAACACCAATACCATGAAAAATTACCTTTTGC
CCGTAGTGGCCCAGGCATTTGCTAGGTGGGCAAAGGAATATAAGGAAGATCAAGAAGATGAAAGG
CCACTAGGACTACGAGATAGACAGTTAGTCATGGGGTGTTGTTGGGCTTTTAGAAGGCACAAGATA
ACATCTATTTATAAGCGCCCGGATACCCAAACCATCATCAAAGTGAACAGCGATTTCCACTCATTCG
TGCTGCCCAGGATAGGCAGTAACACATTGGAGATCGGGCTGAGAACAAGAATCAGGAAAATGTTAG
AGGAGCACAAGGAGCCGTCACCTCTCATTACCGCCGAGGACGTACAAGAAGCTAAGTGCGCAGCCG
ATGAGGCTAAGGAGGTGCGTGAAGCCGAGGAGTTGCGCGCAGCTCTACCACCTTTGGCAGCTGATG
TTGAGGAGCCCACTCTGGAAGCCGATGTCGACTTGATGTTACAAGAGGCTGGGGCCGGCTCAGTGG
AGACACCTCGTGGCTTGATAAAGGTTACCAGCTACGATGGCGAGGACAAGATCGGCTCTTACGCTG
TGCTTTCTCCGCAGGCTGTACTCAAGAGTGAAAAATTATCTTGCATCCACCCTCTCGCTGAACAAGT
CATAGTGATAACACACTCTGGCCGAAAAGGGCGTTATGCCGTGGAACCATACCATGGTAAAGTAGT
GGTGCCAGAGGGACATGCAATACCCGTCCAGGACTTTCAAGCTCTGAGTGAAAGTGCCACCATTGT
GTACAACGAACGTGAGTTCGTAAACAGGTACCTGCACCATATTGCCACACATGGAGGAGCGCTGAA
CACTGATGAAGAATATTACAAAACTGTCAAGCCCAGCGAGCACGACGGCGAATACCTGTACGACAT
CGACAGGAAACAGTGCGTCAAGAAAGAACTAGTCACTGGGCTAGGGCTCACAGGCGAGCTGGTGG
ATCCTCCCTTCCATGAATTCGCCTACGAGAGTCTGAGAACACGACCAGCCGCTCCTTACCAAGTACC
AACCATAGGGGTGTATGGCGTGCCAGGATCAGGCAAGTCTGGCATCATTAAAAGCGCAGTCACCAA
AAAAGATCTAGTGGTGAGCGCCAAGAAAGAAAACTGTGCAGAAATTATAAGGGACGTCAAGAAAA
TGAAAGGGCTGGACGTCAATGCCAGAACTGTGGACTCAGTGCTCTTGAATGGATGCAAACACCCCG
TAGAGACCCTGTATATTGACGAAGCTTTTGCTTGTCATGCAGGTACTCTCAGAGCGCTCATAGCCAT
TATAAGACCTAAAAAGGCAGTGCTCTGCGGGGATCCCAAACAGTGCGGTTTTTTTAACATGATGTGC
CTGAAAGTGCATTTTAACCACGAGATTTGCACACAAGTCTTCCACAAAAGCATCTCTCGCCGTTGCA
CTAAATCTGTGACTTCGGTCGTCTCAACCTTGTTTTACGACAAAAAAATGAGAACGACGAATCCGAA
AGAGACTAAGATTGTGATTGACACTACCGGCAGTACCAAACCTAAGCAGGACGATCTCATTCTCACT
TGTTTCAGAGGGTGGGTGAAGCAGTTGCAAATAGATTACAAAGGCAACGAAATAATGACGGCAGCT
GCCTCTCAAGGGCTGACCCGTAAAGGTGTGTATGCCGTTCGGTACAAGGTGAATGAAAATCCTCTGT
ACGCACCCACCTCAGAACATGTGAACGTCCTACTGACCCGCACGGAGGACCGCATCGTGTGGAAAA
CACTAGCCGGCGACCCATGGATAAAAACACTGACTGCCAAGTACCCTGGGAATTTCACTGCCACGA
TAGAGGAGTGGCAAGCAGAGCATGATGCCATCATGAGGCACATCTTGGAGAGACCGGACCCTACCG
ACGTCTTCCAGAATAAGGCAAACGTGTGTTGGGCCAAGGCTTTAGTGCCGGTGCTGAAGACCGCTG
GCATAGACATGACCACTGAACAATGGAACACTGTGGATTATTTTGAAACGGACAAAGCTCACTCAG
CAGAGATAGTATTGAACCAACTATGCGTGAGGTTCTTTGGACTCGATCTGGACTCCGGTCTATTTTCT
GCACCCACTGTTCCGTTATCCATTAGGAATAATCACTGGGATAACTCCCCGTCGCCTAACATGTACG
GGCTGAATAAAGAAGTGGTCCGTCAGCTCTCTCGCAGGTACCCACAACTGCCTCGGGCAGTTGCCAC
TGGAAGAGTCTATGACATGAACACTGGTACACTGCGCAATTATGATCCGCGCATAAACCTAGTACCT
GTAAACAGAAGACTGCCTCATGCTTTAGTCCTCCACCATAATGAACACCCACAGAGTGACTTTTCTT
CATTCGTCAGCAAATTGAAGGGCAGAACTGTCCTGGTGGTCGGGGAAAAGTTGTCCGTCCCAGGCA
AAATGGTTGACTGGTTGTCAGACCGGCCTGAGGCTACCTTCAGAGCTCGGCTGGATTTAGGCATCCC
AGGTGATGTGCCCAAATATGACATAATATTTGTTAATGTGAGGACCCCATATAAATACCATCACTAT
CAGCAGTGTGAAGACCATGCCATTAAGCTTAGCATGTTGACCAAGAAAGCTTGTCTGCATCTGAATC
CCGGCGGAACCTGTGTCAGCATAGGTTATGGTTACGCTGACAGGGCCAGCGAAAGCATCATTGGTG
CTATAGCGCGGCAGTTCAAGTTTTCCCGGGTATGCAAACCGAAATCCTCACTTGAAGAGACGGAAG
TTCTGTTTGTATTCATTGGGTACGATCGCAAGGCCCGTACGCACAATCCTTACAAGCTTTCATCAACC
TTGACCAACATTTATACAGGTTCCAGACTCCACGAAGCCGGATGTGCACCCTCATATCATGTGGTGC
GAGGGGATATTGCCACGGCCACCGAAGGAGTGATTATAAATGCTGCTAACAGCAAAGGACAACCTG
GCGGAGGGGTGTGCGGAGCGCTGTATAAGAAATTCCCGGAAAGCTTCGATTTACAGCCGATCGAAG
TAGGAAAAGCGCGACTGGTCAAAGGTGCAGCTAAACATATCATTCATGCCGTAGGACCAAACTTCA
ACAAAGTTTCGGAGGTTGAAGGTGACAAACAGTTGGCAGAGGCTTATGAGTCCATCGCTAAGATTG
TCAACGATAACAATTACAAGTCAGTAGCGATTCCACTGTTGTCCACCGGCATCTTTTCCGGGAACAA
AGATCGACTAACCCAATCATTGAACCATTTGCTGACAGCTTTAGACACCACTGATGCAGATGTAGCC
ATATACTGCAGGGACAAGAAATGGGAAATGACTCTCAAGGAAGCAGTGGCTAGGAGAGAAGCAGT
GGAGGAGATATGCATATCCGACGACTCTTCAGTGACAGAACCTGATGCAGAGCTGGTGAGGGTGCA
TCCGAAGAGTTCTTTGGCTGGAAGGAAGGGCTACAGCACAAGCGATGGCAAAACTTTCTCATATTTG
GAAGGGACCAAGTTTCACCAGGCGGCCAAGGATATAGCAGAAATTAATGCCATGTGGCCCGTTGCA
ACGGAGGCCAATGAGCAGGTATGCATGTATATCCTCGGAGAAAGCATGAGCAGTATTAGGTCGAAA
TGCCCCGTCGAAGAGTCGGAAGCCTCCACACCACCTAGCACGCTGCCTTGCTTGTGCATCCATGCCA
TGACTCCAGAAAGAGTACAGCGCCTAAAAGCCTCACGTCCAGAACAAATTACTGTGTGCTCATCCTT
TCCATTGCCGAAGTATAGAATCACTGGTGTGCAGAAGATCCAATGCTCCCAGCCTATATTGTTCTCA
CCGAAAGTGCCTGCGTATATTCATCCAAGGAAGTATCTCGTGGAAACACCACCGGTAGACGAGACT
CCGGAGCCATCGGCAGAGAACCAATCCACAGAGGGGACACCTGAACAACCACCACTTATAACCGAG
GATGAGACCAGGACTAGAACGCCTGAGCCGATCATCATCGAAGAGGAAGAAGAGGATAGCATAAG
TTTGCTGTCAGATGGCCCGACCCACCAGGTGCTGCAAGTCGAGGCAGACATTCACGGGCCGCCCTCT
GTATCTAGCTCATCCTGGTCCATTCCTCATGCATCCGACTTTGATGTGGACAGTTTATCCATACTTGA
CACCCTGGAGGGAGCTAGCGTGACCAGCGGGGCAACGTCAGCCGAGACTAACTCTTACTTCGCAAA
GAGTATGGAGTTTCTGGCGCGACCGGTGCCTGCGCCTCGAACAGTATTCAGGAACCCTCCACATCCC
GCTCCGCGCACAAGAACACCGTCACTTGCACCCAGCAGGGCCTGCTCGAGAACCAGCCTAGTTTCC
ACCCCGCCAGGCGTGAATAGGGTGATCACTAGAGAGGAGCTCGAGGCGCTTACCCCGTCACGCACT
CCTAGCAGGTCGGTCTCGAGAACCAGCCTGGTCTCCAACCCGCCAGGCGTAAATAGGGTGATTACA
AGAGAGGAGTTTGAGGCGTTCGTAGCACAACAACAATGACGGTTTGATGCGGGTGCATACATCTTTT
CCTCCGACACCGGTCAAGGGCATTTACAACAAAAATCAGTAAGGCAAACGGTGCTATCCGAAGTGG
TGTTGGAGAGGACCGAATTGGAGATTTCGTATGCCCCGCGCCTCGACCAAGAAAAAGAAGAATTAC
TACGCAAGAAATTACAGTTAAATCCCACACCTGCTAACAGAAGCAGATACCAGTCCAGGAAGGTGG
AGAACATGAAAGCCATAACAGCTAGACGTATTCTGCAAGGCCTAGGGCATTATTTGAAGGCAGAAG
GAAAAGTGGAGTGCTACCGAACCCTGCATCCTGTTCCTTTGTATTCATCTAGTGTGAACCGTGCCTTT
TCAAGCCCCAAGGTCGCAGTGGAAGCCTGTAACGCCATGTTGAAAGAGAACTTTCCGACTGTGGCTT
CTTACTGTATTATTCCAGAGTACGATGCCTATTTGGACATGGTTGACGGAGCTTCATGCTGCTTAGAC
ACTGCCAGTTTTTGCCCTGCAAAGCTGCGCAGCTTTCCAAAGAAACACTCCTATTTGGAACCCACAA
TACGATCGGCAGTGCCTTCAGCGATCCAGAACACGCTCCAGAACGTCCTGGCAGCTGCCACAAAAA
GAAATTGCAATGTCACGCAAATGAGAGAATTGCCCGTATTGGATTCGGCGGCCTTTAATGTGGAATG
CTTCAAGAAATATGCGTGTAATAATGAATATTGGGAAACGTTTAAAGAAAACCCCATCAGGCTTACT
GAAGAAAACGTGGTAAATTACATTACCAAATTAAAAGGACCAAAAGCTGCTGCTCTTTTTGCGAAG
ACACATAATTTGAATATGTTGCAGGACATACCAATGGACAGGTTTGTAATGGACTTAAAGAGAGAC
GTGAAAGTGACTCCAGGAACAAAACATACTGAAGAACGGCCCAAGGTACAGGTGATCCAGGCTGCC
GATCCGCTAGCAACAGCGTATCTGTGCGGAATCCACCGAGAGCTGGTTAGGAGATTAAATGCGGTC
CTGCTTCCGAACATTCATACACTGTTTGATATGTCGGCTGAAGACTTTGACGCTATTATAGCCGAGC
ACTTCCAGCCTGGGGATTGTGTTCTGGAAACTGACATCGCGTCGTTTGATAAAAGTGAGGACGACGC
CATGGCTCTGACCGCGTTAATGATTCTGGAAGACTTAGGTGTGGACGCAGAGCTGTTGACGCTGATT
GAGGCGGCTTTCGGCGAAATTTCATCAATACATTTGCCCACTAAAACTAAATTTAAATTCGGAGCCA
TGATGAAATCTGGAATGTTCCTCACACTGTTTGTGAACACAGTCATTAACATTGTAATCGCAAGCAG
AGTGTTGAGAGAACGGCTAACCGGATCACCATGTGCAGCATTCATTGGAGATGACAATATCGTGAA
AGGAGTCAAATCGGACAAATTAATGGCAGACAGGTGCGCCACCTGGTTGAATATGGAAGTCAAGAT
TATAGATGCTGTGGTGGGCGAGAAAGCGCCTTATTTCTGTGGAGGGTTTATTTTGTGTGACTCCGTG
ACCGGCACAGCGTGCCGTGTGGCAGACCCCCTAAAAAGGCTGTTTAAGCTTGGCAAACCTCTGGCA
GCAGACGATGAACATGATGATGACAGGAGAAGGGCATTGCATGAAGAGTCAACACGCTGGAACCG
AGTGGGTATTCTTTCAGAGCTGTGCAAGGCAGTAGAATCAAGGTATGAAACCGTAGGAACTTCCAT
CATAGTTATGGCCATGACTACTCTAGCTAGCAGTGTTAAATCATTCAGCTACCTGAGAGGGGCCCCT
ATAACTCTCTACGGCTAACCTGAATGGACTACGACATAGTCTAGTCCGCCAAGATGTTCCCGTTCCA
GCCAATGTATCCGATGCAGCCAATGCCCTATCGCAACCCGTTCGCGGCCCCGCGCAGGCCCTGGTTC
CCCAGAACCGACCCTTTTCTGGCGATGCAGGTGCAGGAATTAACCCGCTCGATGGCTAACCTGACGT
TCAAGCAACGCCGGGACGCGCCACCTGAGGGGCCATCCGCTAAGAAACCGAAGAAGGAGGCCTCG
CAAAAACAGAAAGGGGGAGGCCAAGGGAAGAAGAAGAAGAACCAAGGGAAGAAGAAGGCTAAGA
CAGGGCCGCCTAATCCGAAGGCACAGAATGGAAACAAGAAGAAGACCAACAAGAAACCAGGCAAG
AGACAGCGCATGGTCATGAAATTGGAATCTGACAAGACGTTCCCAATCATGTTGGAAGGGAAGATA
AACGGCTACGCTTGTGTGGTCGGAGGGAAGTTATTCAGGCCGATGCATGTGGAAGGCAAGATCGAC
AACGACGTTCTGGCCGCGCTTAAGACGAAGAAAGCATCCAAATACGATCTTGAGTATGCAGATGTG
CCACAGAACATGCGGGCCGATACATTCAAATACACCCATGAGAAACCCCAAGGCTATTACAGCTGG
CATCATGGAGCAGTCCAATATGAAAATGGGCGTTTCACGGTGCCGAAAGGAGTTGGGGCCAAGGGA
GACAGCGGACGACCCATTCTGGATAACCAGGGACGGGTGGTCGCTATTGTGCTGGGAGGTGTGAAT
GAAGGATCTAGGACAGCCCTTTCAGTCGTCATGTGGAACGAGAAGGGAGTTACCGTGAAGTATACT
CCGGAGAACTGCGAGCAATGGTCACTAGTGACCACCATGTGTCTGCTCGCCAATGTGACGTTCCCAT
GTGCTCAACCACCAATTTGCTACGACAGAAAACCAGCAGAGACTTTGGCCATGCTCAGCGTTAACGT
TGACAACCCGGGCTACGATGAGCTGCTGGAAGCAGCTGTTAAGTGCCCCGGAAGGAAAAGGAGATC
CACCGAGGAGCTGTTTAATGAGTATAAGCTAACGCGCCCTTACATGGCCAGATGCATCAGATGTGC
AGTTGGGAGCTGCCATAGTCCAATAGCAATCGAGGCAGTAAAGAGCGACGGGCACGACGGTTATGT
TAGACTTCAGACTTCCTCGCAGTATGGCCTGGATTCCTCCGGCAACTTAAAGGGCAGGACCATGCGG
TATGACATGCACGGGACCATTAAAGAGATACCACTACATCAAGTGTCACTCTATACATCTCGCCCGT
GTCACATTGTGGATGGGCACGGTTATTTCCTGCTTGCCAGGTGCCCGGCAGGGGACTCCATCACCAT
GGAATTTAAGAAAGATTCCGTCAGACACTCCTGCTCGGTGCCGTATGAAGTGAAATTTAATCCTGTA
GGCAGAGAACTCTATACTCATCCCCCAGAACACGGAGTAGAGCAAGCGTGCCAAGTCTACGCACAT
GATGCACAGAACAGAGGAGCTTATGTCGAGATGCACCTCCCGGGCTCAGAAGTGGACAGCAGTTTG
GTTTCCTTGAGCGGCAGTTCAGTCACCGTGACACCTCCTGATGGGACTAGCGCCCTGGTGGAATGCG
AGTGTGGCGGCACAAAGATCTCCGAGACCATCAACAAGACAAAACAGTTCAGCCAGTGCACAAAG
AAGGAGCAGTGCAGAGCATATCGGCTGCAGAACGATAAGTGGGTGTATAATTCTGACAAACTGCCC
AAAGCAGCGGGAGCCACCTTAAAAGGAAAACTGCATGTCCCATTCTTGCTGGCAGACGGCAAATGC
ACCGTGCCTCTAGCACCAGAACCTATGATAACCTTCGGTTTCAGATCAGTGTCACTGAAACTGCACC
CTAAGAATCCCACATATCTAATCACCCGCCAACTTGCTGATGAGCCTCACTACACGCACGAGCTCAT
ATCTGAACCAGCTGTTAGGAATTTTACCGTCACCGAAAAAGGGTGGGAGTTTGTATGGGGAAACCA
CCCGCCGAAAAGGTTTTGGGCACAGGAAACAGCACCCGGAAATCCACATGGGCTACCGCACGAGGT
GATAACTCATTATTACCACAGATACCCTATGTCCACCATCCTGGGTTTGTCAATTTGTGCCGCCATTG
CAACCGTTTCCGTTGCAGCGTCTACCTGGCTGTTTTGCAGATCTAGAGTTGCGTGCCTAACTCCTTAC
CGGCTAACACCTAACGCTAGGATACCATTTTGTCTGGCTGTGCTTTGCTGCGCCCGCACTGCCCGGG
CCGAGACCACCTGGGAGTCCTTGGATCACCTATGGAACAATAACCAACAGATGTTCTGGATTCAATT
GCTGATCCCTCTGGCCGCCTTGATCGTAGTGACTCGCCTGCTCAGGTGCGTGTGCTGTGTCGTGCCTT
TTTTAGTCATGGCCGGCGCCGCAGGCGCCGGCGCCTACGAGCACGCGACCACGATGCCGAGCCAAG
CGGGAATCTCGTATAACACTATAGTCAACAGAGCAGGCTACGCACCACTCCCTATCAGCATAACAC
CAACAAAGATCAAGCTGATACCTACAGTGAACTTGGAGTACGTCACCTGCCACTACAAAACAGGAA
TGGATTCACCAGCCATCAAATGCTGCGGATCTCAGGAATGCACTCCAACTTACAGGCCTGATGAACA
GTGCAAAGTCTTCACAGGGGTTTACCCGTTCATGTGGGGTGGTGCATATTGCTTTTGCGACACTGAG
AACACCCAAGTCAGCAAGGCCTACGTAATGAAATCTGACGACTGCCTTGCGGATCATGCTGAAGCA
TATAAAGCGCACACAGCCTCAGTGCAGGCGTTCCTCAACATCACAGTGGGAGAACACTCTATTGTG
ACTACCGTGTATGTGAATGGAGAAACTCCTGTGAATTTCAATGGGGTCAAAATAACTGCAGGTCCGC
TTTCCACAGCTTGGACACCCTTTGATCGCAAAATCGTGCAGTATGCCGGGGAGATCTATAATTATGA
TTTTCCTGAGTATGGGGCAGGACAACCAGGAGCATTTGGAGATATACAATCCAGAACAGTCTCAAG
CTCTGATCTGTATGCCAATACCAACCTAGTGCTGCAGAGACCCAAAGCAGGAGCGATCCACGTGCC
ATACACTCAGGCACCTTCGGGTTTTGAGCAATGGAAGAAAGATAAAGCTCCATCATTGAAATTTACC
GCCCCTTTCGGATGCGAAATATATACAAACCCCATTCGCGCCGAAAACTGTGCTGTAGGGTCAATTC
CATTAGCCTTTGACATTCCCGACGCCTTGTTCACCAGGGTGTCAGAAACACCGACACTTTCAGCGGC
CGAATGCACTCTTAACGAGTGCGTGTATTCTTCCGACTTTGGTGGGATCGCCACGGTCAAGTACTCG
GCCAGCAAGTCAGGCAAGTGCGCAGTCCATGTGCCATCAGGGACTGCTACCCTAAAAGAAGCAGCA
GTCGAGCTAACCGAGCAAGGGTCGGCGACTATCCATTTCTCGACCGCAAATATCCACCCGGAGTTCA
GGCTCCAAATATGCACATCATATGTTACGTGCAAAGGTGATTGTCACCCCCCGAAAGACCATATTGT
GACACACCCTCAGTATCACGCCCAAACATTTACAGCCGCGGTGTCAAAAACCGCGTGGACGTGGTT
AACATCCCTGCTGGGAGGATCAGCCGTAATTATTATAATTGGCTTGGTGCTGGCTACTATTGTGGCC
ATGTACGTGCTGACCAACCAGAAACATAATTGAATACAGCAGCAATTGGCAAGCTGCTTACATAGA
ACTCGCGGCGATTGGCATGCCGCCTTAAAATTTTTATTTTATTTTTCTTTTCTTTTCCGAATCGGATTT
TGTTTTTAATATTTC
VEE Delivery Vector (SEQ ID NO: 6); VEE genome with nucleotides 7544-11175 deleted [alphavirus
structural proteins removed]
ATGggcggcgcatgagagaagcccagaccaattacctacccaaaATGGagaaagttcacgttgacatcgaggaagacagcccattcctcagagct
ttgcagcggagcttcccgcagtttgaggtagaagccaagcaggtcactgataatgaccatgctaatgccagagcgttttcgcatctggcttcaaa
actgatcgaaacggaggtggacccatccgacacgatccttgacattggaagtgcgcccgcccgcagaatgtattctaagcacaagtatcattgta
tctgtccgatgagatgtgcggaagatccggacagattgtataagtatgcaactaagctgaagaaaaactgtaaggaaataactgataaggaattg
gacaagaaaatgaaggagctcgccgccgtcatgagcgaccctgacctggaaactgagactatgtgcctccacgacgacgagtcgtgtcgctacga
agggcaagtcgctgtttaccaggatgtatacgcggttgacggaccgacaagtctctatcaccaagccaataagggagttagagtcgcctactgga
taggctttgacaccaccccttttatgtttaagaacttggctggagcatatccatcatactctaccaactgggccgacgaaaccgtgttaacggct
cgtaacataggcctatgcagctctgacgttatggagcggtcacgtagagggatgtccattcttagaaagaagtatttgaaaccatccaacaatgt
tctattctctgttggctcgaccatctaccacgagaagagggacttactgaggagctggcacctgccgtctgtatttcacttacgtggcaagcaaa
attacacatgtcggtgtgagactatagttagttgcgacgggtacgtcgttaaaagaatagctatcagtccaggcctgtatgggaagccttcaggc
tatgctgctacgatgcaccgcgagggattcttgtgctgcaaagtgacagacacattgaacggggagagggtctcttttcccgtgtgcacgtatgt
gccagctacattgtgtgaccaaatgactggcatactggcaacagatgtcagtgcggacgacgcgcaaaaactgctggttgggctcaaccagcgta
tagtcgtcaacggtcgcacccagagaaacaccaataccatgaaaaattaccttttgcccgtagtggcccaggcatttgctaggtgggcaaaggaa
tataaggaagatcaagaagatgaaaggccactaggactacgagatagacagttagtcatggggtgttgttgggcttttagaaggcacaagataac
atctatttataagcgcccggatacccaaaccatcatcaaagtgaacagcgatttccactcattcgtgctgcccaggataggcagtaacacattgg
agatcgggctgagaacaagaatcaggaaaatgttagaggagcacaaggagccgtcacctctcattaccgccgaggacgtacaagaagctaagtgc
gcagccgatgaggctaaggaggtgcgtgaagccgaggagttgcgcgcagctctaccacctttggcagctgatgttgaggagcccactctggaagc
cgatgtcgacttgatgttacaagaggctggggccggctcagtggagacacctcgtggcttgataaaggttaccagctacgctggcgaggacaaga
tcggctcttacgctgtgctttctccgcaggctgtactcaagagtgaaaaattatcttgcatccaccctctcgctgaacaagtcatagtgataaca
cactctggccgaaaagggcgttatgccgtggaaccataccatggtaaagtagtggtgccagagggacatgcaatacccgtccaggactttcaagc
tctgagtgaaagtgccaccattgtgtacaacgaacgtgagttcgtaaacaggtacctgcaccatattgccacacatggaggagcgctgaacactg
atgaagaatattacaaaactgtcaagcccagcgagcacgacggcgaatacctgtacgacatcgacaggaaacagtgcgtcaagaaagaactagtc
actgggctagggctcacaggcgagctggtggatcctcccttccatgaattcgcctacgagagtctgagaacacgaccagccgctccttaccaagt
accaaccataggggtgtatggcgtgccaggatcaggcaagtctggcatcattaaaagcgcagtcaccaaaaaagatctagtggtgagcgccaaga
aagaaaactgtgcagaaattataagggacgtcaagaaaatgaaagggctggacgtcaatgccagaactgtggactcagtgctcttgaatggatgc
aaacaccccgtagagaccctgtatattgacgaagcttttgcttgtcatgcaggtactctcagagcgctcatagccattataagacctaaaaaggc
agtgctctgcggggatcccaaacagtgcggtttttttaacatgatgtgcctgaaagtgcattttaaccacgagatttgcacacaagtcttccaca
aaagcatctctcgccgttgcactaaatctgtgacttcggtcgtctcaaccttgttttacgacaaaaaaatgagaacgacgaatccgaaagagact
aagattgtgattgacactaccggcagtaccaaacctaagcaggacgatctcattctcacttgtttcagaggggggtgaagcagttgcaaatagat
tacaaaggcaacgaaataatgacggcagctgcctctcaagggctgacccgtaaaggtgtgtatgccgttcggtacaaggtgaatgaaaatcctct
gtacgcacccacctcagaacatgtgaacgtcctactgacccgcacggaggaccgcatcgtgtggaaaacactagccggcgacccatggataaaaa
cactgactgccaagtaccctgggaatttcactgccacgatagaggagtggcaagcagagcatgatgccatcatgaggcacatcttggagagaccg
gaccctaccgacgtcttccagaataaggcaaacgtgtgttgggccaaggctttagtgccggtgctgaagaccgctggcatagacatgaccactga
acaatggaacactgtggattattttgaaacggacaaagctcactcagcagagatagtattgaaccaactatgcgtgaggttctttggactcgatc
tggactccggtctattttctgcacccactgttccgttatccattaggaataatcactgggataactccccgtcgcctaacatgtacgggctgaat
aaagaagtggtccgtcagctctctcgcaggtacccacaactgcctcgggcagttgccactggaagagtctatgacatgaacactggtacactgcg
caattatgatccgcgcataaacctagtacctgtaaacagaagactgcctcatgctttagtcctccaccataatgaacacccacagagtgactttt
cttcattcgtcagcaaattgaagggcagaactgtcctggtggtcggggaaaagttgtccgtcccaggcaaaatggttgactggttgtcagaccgg
cctgaggctaccttcagagctcggctggatttaggcatcccaggtgatgtgcccaaatatgacataatatttgttaatgtgaggaccccatataa
ataccatcactatcagcagtgtgaagaccatgccattaagcttagcatgttgaccaagaaagcttgtctgcatctgaatcccggcggaacctgtg
tcagcataggttatggttacgctgacagggccagcgaaagcatcattggtgctatagcgcggcagttcaagttttcccgggtatgcaaaccgaaa
tcctcacttgaagagacggaagttctgtttgtattcattgggtacgatcgcaaggcccgtacgcacaatccttacaagctttcatcaaccttgac
caacatttatacaggttccagactccacgaagccggatgtgcaccctcatatcatgtggtgcgaggggatattgccacggccaccgaaggagtga
ttataaatgctgctaacagcaaaggacaacctggcggaggggtgtgcggagcgctgtataagaaattcccggaaagcttcgatttacagccgatc
gaagtaggaaaagcgcgactggtcaaaggtgcagctaaacatatcattcatgccgtaggaccaaacttcaacaaagtttcggaggttgaaggtga
caaacagttggcagaggcttatgagtccatcgctaagattgtcaacgataacaattacaagtcagtagcgattccactgttgtccaccggcatct
tttccgggaacaaagatcgactaacccaatcattgaaccatttgctgacagctttagacaccactgatgcagatgtagccatatactgcagggac
aagaaatgggaaatgactctcaaggaagcagtggctaggagagaagcagtggaggagatatgcatatccgacgactcttcagtgacagaacctga
tgcagagctggtgagggtgcatccgaagagttctttggctggaaggaagggctacagcacaagcgatggcaaaactttctcatatttggaaggga
ccaagtttcaccaggcggccaaggatatagcagaaattaatgccatgtggcccgttgcaacggaggccaatgagcaggtatgcatgtatatcctc
ggagaaagcatgagcagtattaggtcgaaatgccccgtcgaagagtcggaagcctccacaccacctagcacgctgccttgcttgtgcatccatgc
catgactccagaaagagtacagcgcctaaaagcctcacgtccagaacaaattactgtgtgctcatcctttccattgccgaagtatagaatcactg
gtgtgcagaagatccaatgctcccagcctatattgttctcaccgaaagtgcctgcgtatattcatccaaggaagtatctcgtggaaacaccaccg
gtagacgagactccggagccatcggcagagaaccaatccacagaggggacacctgaacaaccaccacttataaccgaggatgagaccaggactag
aacgcctgagccgatcatcatcgaagaggaagaagaggatagcataagtttgctgtcagatggcccgacccaccaggtgctgcaagtcgaggcag
acattcacgggccgccctctgtatctagctcatcctggtccattcctcatgcatccgactttgatgtggacagtttatccatacttgacaccctg
gagggagctagcgtgaccagcggggcaacgtcagccgagactaactcttacttcgcaaagagtatggagtttctggcgcgaccggtgcctgcgcc
tcgaacagtattcaggaaccctccacatcccgctccgcgcacaagaacaccgtcacttgcacccagcagggcctgctcgagaaccagcctagttt
ccaccccgccaggcgtgaatagggtgatcactagagaggagctcgaggcgcttaccccgtcacgcactcctagcaggtcggtctcgagaaccagc
ctggtctccaacccgccaggcgtaaatagggtgattacaagagaggagtttgaggcgttcgtagcacaacaacaatgacggtttgatgcgggtgc
atacatcttttcctccgacaccggtcaagggcatttacaacaaaaatcagtaaggcaaacggtgctatccgaagtggtgttggagaggaccgaat
tggagatttcgtatgccccgcgcctcgaccaagaaaaagaagaattactacgcaagaaattacagttaaatcccacacctgctaacagaagcaga
taccagtccaggaaggtggagaacatgaaagccataacagctagacgtattctgcaaggcctagggcattatttgaaggcagaaggaaaagtgga
gtgctaccgaaccctgcatcctgttcctttgtattcatctagtgtgaaccgtgccttttcaagccccaaggtcgcagtggaagcctgtaacgcca
tgttgaaagagaactttccgactgtggcttcttactgtattattccagagtacgatgcctatttggacatggttgacggagcttcatgctgctta
gacactgccagtttttgccctgcaaagctgcgcagctttccaaagaaacactcctatttggaacccacaatacgatcggcagtgccttcagcgat
ccagaacacgctccagaacgtcctggcagctgccacaaaaagaaattgcaatgtcacgcaaatgagagaattgcccgtattggattcggcggcct
ttaatgtggaatgcttcaagaaatatgcgtgtaataatgaatattgggaaacgtttaaagaaaaccccatcaggcttactgaagaaaacgtggta
aattacattaccaaattaaaaggaccaaaagctgctgctctttttgcgaagacacataatttgaatatgttgcaggacataccaatggacaggtt
tgtaatggacttaaagagagacgtgaaagtgactccaggaacaaaacatactgaagaacggcccaaggtacaggtgatccaggctgccgatccgc
tagcaacagcgtatctgtgcggaatccaccgagagctggttaggagattaaatgcggtcctgcttccgaacattcatacactgtttgatatgtcg
gctgaagactttgacgctattatagccgagcacttccagcctggggattgtgttctggaaactgacatcgcgtcgtttgataaaagtgaggacga
cgccatggctctgaccgcgttaatgattctggaagacttaggtgtggacgcagagctgttgacgctgattgaggcggctttcggcgaaatttcat
caatacatttgcccactaaaactaaatttaaattcggagccatgatgaaatctggaatgttcctcacactgtttgtgaacacagtcattaacatt
gtaatcgcaagcagagtgttgagagaacggctaaccggatcaccatgtgcagcattcattggagatgacaatatcgtgaaaggagtcaaatcgga
caaattaatggcagacaggtgcgccacctggttgaatatggaagtcaagattatagatgctgtggtgggcgagaaagcgccttatttctgtggag
ggtttattttgtgtgactccgtgaccggcacagcgtgccgtgtggcagaccccctaaaaaggctgtttaagcttggcaaacctctggcagcagac
gatgaacatgatgatgacaggagaagggcattgcatgaagagtcaacacgctggaaccgagtgggtattctttcagagctgtgcaaggcagtaga
atcaaggtatgaaaccgtaggaacttccatcatagttatggccatgactactctagctagcagtgttaaatcattcagctacctgagaggggccc
ctataactctctacggcTAAcctgaatggactacgacgtatcacgcccaaacatttacagccgcggtgtcaaaaaccgcgtggacgtggttaaca
tccctgctgggaggatcagccgtaattattataattggcttggtgctggctactattgtggccatgtacgtgctgaccaaccagaaacataattg
aatacagcagcaattggcaagctgcttacatagaactcgcggcgattggcatgccgccttaaaatttttattttattttttcttttcttttccga
atcggattttgtttttaatatttcAAAAAAAAAAAAAAAAAAAAAAAAAAAAAAAAAAAAAAAAAAAAAAAAAAAAAAAAAAAAAAAAAAAAAAA
AAAAAAAAA
TC-83 Delivery Vector (SEQ ID NO: 7); TC-83 genome with nucleotides 7544-11175 deleted
[alphavirus structural proteins removed]
ATAGGCGGCGCATGAGAGAAGCCCAGACCAATTACCTACCCAAAATGGAGAAAGTTCACGTTGACA
TCGAGGAAGACAGCCCATTCCTCAGAGCTTTGCAGCGGAGCTTCCCGCAGTTTGAGGTAGAAGCCA
AGCAGGTCACTGATAATGACCATGCTAATGCCAGAGCGTTTTCGCATCTGGCTTCAAAACTGATCGA
AACGGAGGTGGACCCATCCGACACGATCCTTGACATTGGAAGTGCGCCCGCCCGCAGAATGTATTC
TAAGCACAAGTATCATTGTATCTGTCCGATGAGATGTGCGGAAGATCCGGACAGATTGTATAAGTAT
GCAACTAAGCTGAAGAAAAACTGTAAGGAAATAACTGATAAGGAATTGGACAAGAAAATGAAGGA
GCTCGCCGCCGTCATGAGCGACCCTGACCTGGAAACTGAGACTATGTGCCTCCACGACGACGAGTC
GTGTCGCTACGAAGGGCAAGTCGCTGTTTACCAGGATGTATACGCGGTTGACGGACCGACAAGTCT
CTATCACCAAGCCAATAAGGGAGTTAGAGTCGCCTACTGGATAGGCTTTGACACCACCCCTTTTATG
TTTAAGAACTTGGCTGGAGCATATCCATCATACTCTACCAACTGGGCCGACGAAACCGTGTTAACGG
CTCGTAACATAGGCCTATGCAGCTCTGACGTTATGGAGCGGTCACGTAGAGGGATGTCCATTCTTAG
AAAGAAGTATTTGAAACCATCCAACAATGTTCTATTCTCTGTTGGCTCGACCATCTACCACGAGAAG
AGGGACTTACTGAGGAGCTGGCACCTGCCGTCTGTATTTCACTTACGTGGCAAGCAAAATTACACAT
GTCGGTGTGAGACTATAGTTAGTTGCGACGGGTACGTCGTTAAAAGAATAGCTATCAGTCCAGGCCT
GTATGGGAAGCCTTCAGGCTATGCTGCTACGATGCACCGCGAGGGATTCTTGTGCTGCAAAGTGACA
GACACATTGAACGGGGAGAGGGTCTCTTTTCCCGTGTGCACGTATGTGCCAGCTACATTGTGTGACC
AAATGACTGGCATACTGGCAACAGATGTCAGTGCGGACGACGCGCAAAAACTGCTGGTTGGGCTCA
ACCAGCGTATAGTCGTCAACGGTCGCACCCAGAGAAACACCAATACCATGAAAAATTACCTTTTGC
CCGTAGTGGCCCAGGCATTTGCTAGGTGGGCAAAGGAATATAAGGAAGATCAAGAAGATGAAAGG
CCACTAGGACTACGAGATAGACAGTTAGTCATGGGGTGTTGTTGGGCTTTTAGAAGGCACAAGATA
ACATCTATTTATAAGCGCCCGGATACCCAAACCATCATCAAAGTGAACAGCGATTTCCACTCATTCG
TGCTGCCCAGGATAGGCAGTAACACATTGGAGATCGGGCTGAGAACAAGAATCAGGAAAATGTTAG
AGGAGCACAAGGAGCCGTCACCTCTCATTACCGCCGAGGACGTACAAGAAGCTAAGTGCGCAGCCG
ATGAGGCTAAGGAGGTGCGTGAAGCCGAGGAGTTGCGCGCAGCTCTACCACCTTTGGCAGCTGATG
TTGAGGAGCCCACTCTGGAAGCCGATGTCGACTTGATGTTACAAGAGGCTGGGGCCGGCTCAGTGG
AGACACCTCGTGGCTTGATAAAGGTTACCAGCTACGATGGCGAGGACAAGATCGGCTCTTACGCTG
TGCTTTCTCCGCAGGCTGTACTCAAGAGTGAAAAATTATCTTGCATCCACCCTCTCGCTGAACAAGT
CATAGTGATAACACACTCTGGCCGAAAAGGGCGTTATGCCGTGGAACCATACCATGGTAAAGTAGT
GGTGCCAGAGGGACATGCAATACCCGTCCAGGACTTTCAAGCTCTGAGTGAAAGTGCCACCATTGT
GTACAACGAACGTGAGTTCGTAAACAGGTACCTGCACCATATTGCCACACATGGAGGAGCGCTGAA
CACTGATGAAGAATATTACAAAACTGTCAAGCCCAGCGAGCACGACGGCGAATACCTGTACGACAT
CGACAGGAAACAGTGCGTCAAGAAAGAACTAGTCACTGGGCTAGGGCTCACAGGCGAGCTGGTGG
ATCCTCCCTTCCATGAATTCGCCTACGAGAGTCTGAGAACACGACCAGCCGCTCCTTACCAAGTACC
AACCATAGGGGTGTATGGCGTGCCAGGATCAGGCAAGTCTGGCATCATTAAAAGCGCAGTCACCAA
AAAAGATCTAGTGGTGAGCGCCAAGAAAGAAAACTGTGCAGAAATTATAAGGGACGTCAAGAAAA
TGAAAGGGCTGGACGTCAATGCCAGAACTGTGGACTCAGTGCTCTTGAATGGATGCAAACACCCCG
TAGAGACCCTGTATATTGACGAAGCTTTTGCTTGTCATGCAGGTACTCTCAGAGCGCTCATAGCCAT
TATAAGACCTAAAAAGGCAGTGCTCTGCGGGGATCCCAAACAGTGCGGTTTTTTTAACATGATGTGC
CTGAAAGTGCATTTTAACCACGAGATTTGCACACAAGTCTTCCACAAAAGCATCTCTCGCCGTTGCA
CTAAATCTGTGACTTCGGTCGTCTCAACCTTGTTTTACGACAAAAAAATGAGAACGACGAATCCGAA
AGAGACTAAGATTGTGATTGACACTACCGGCAGTACCAAACCTAAGCAGGACGATCTCATTCTCACT
TGTTTCAGAGGGTGGGTGAAGCAGTTGCAAATAGATTACAAAGGCAACGAAATAATGACGGCAGCT
GCCTCTCAAGGGCTGACCCGTAAAGGTGTGTATGCCGTTCGGTACAAGGTGAATGAAAATCCTCTGT
ACGCACCCACCTCAGAACATGTGAACGTCCTACTGACCCGCACGGAGGACCGCATCGTGTGGAAAA
CACTAGCCGGCGACCCATGGATAAAAACACTGACTGCCAAGTACCCTGGGAATTTCACTGCCACGA
TAGAGGAGTGGCAAGCAGAGCATGATGCCATCATGAGGCACATCTTGGAGAGACCGGACCCTACCG
ACGTCTTCCAGAATAAGGCAAACGTGTGTTGGGCCAAGGCTTTAGTGCCGGTGCTGAAGACCGCTG
GCATAGACATGACCACTGAACAATGGAACACTGTGGATTATTTTGAAACGGACAAAGCTCACTCAG
CAGAGATAGTATTGAACCAACTATGCGTGAGGTTCTTTGGACTCGATCTGGACTCCGGTCTATTTTCT
GCACCCACTGTTCCGTTATCCATTAGGAATAATCACTGGGATAACTCCCCGTCGCCTAACATGTACG
GGCTGAATAAAGAAGTGGTCCGTCAGCTCTCTCGCAGGTACCCACAACTGCCTCGGGCAGTTGCCAC
TGGAAGAGTCTATGACATGAACACTGGTACACTGCGCAATTATGATCCGCGCATAAACCTAGTACCT
GTAAACAGAAGACTGCCTCATGCTTTAGTCCTCCACCATAATGAACACCCACAGAGTGACTTTTCTT
CATTCGTCAGCAAATTGAAGGGCAGAACTGTCCTGGTGGTCGGGGAAAAGTTGTCCGTCCCAGGCA
AAATGGTTGACTGGTTGTCAGACCGGCCTGAGGCTACCTTCAGAGCTCGGCTGGATTTAGGCATCCC
AGGTGATGTGCCCAAATATGACATAATATTTGTTAATGTGAGGACCCCATATAAATACCATCACTAT
CAGCAGTGTGAAGACCATGCCATTAAGCTTAGCATGTTGACCAAGAAAGCTTGTCTGCATCTGAATC
CCGGCGGAACCTGTGTCAGCATAGGTTATGGTTACGCTGACAGGGCCAGCGAAAGCATCATTGGTG
CTATAGCGCGGCAGTTCAAGTTTTCCCGGGTATGCAAACCGAAATCCTCACTTGAAGAGACGGAAG
TTCTGTTTGTATTCATTGGGTACGATCGCAAGGCCCGTACGCACAATCCTTACAAGCTTTCATCAACC
TTGACCAACATTTATACAGGTTCCAGACTCCACGAAGCCGGATGTGCACCCTCATATCATGTGGTGC
GAGGGGATATTGCCACGGCCACCGAAGGAGTGATTATAAATGCTGCTAACAGCAAAGGACAACCTG
GCGGAGGGGTGTGCGGAGCGCTGTATAAGAAATTCCCGGAAAGCTTCGATTTACAGCCGATCGAAG
TAGGAAAAGCGCGACTGGTCAAAGGTGCAGCTAAACATATCATTCATGCCGTAGGACCAAACTTCA
ACAAAGTTTCGGAGGTTGAAGGTGACAAACAGTTGGCAGAGGCTTATGAGTCCATCGCTAAGATTG
TCAACGATAACAATTACAAGTCAGTAGCGATTCCACTGTTGTCCACCGGCATCTTTTCCGGGAACAA
AGATCGACTAACCCAATCATTGAACCATTTGCTGACAGCTTTAGACACCACTGATGCAGATGTAGCC
ATATACTGCAGGGACAAGAAATGGGAAATGACTCTCAAGGAAGCAGTGGCTAGGAGAGAAGCAGT
GGAGGAGATATGCATATCCGACGACTCTTCAGTGACAGAACCTGATGCAGAGCTGGTGAGGGTGCA
TCCGAAGAGTTCTTTGGCTGGAAGGAAGGGCTACAGCACAAGCGATGGCAAAACTTTCTCATATTTG
GAAGGGACCAAGTTTCACCAGGCGGCCAAGGATATAGCAGAAATTAATGCCATGTGGCCCGTTGCA
ACGGAGGCCAATGAGCAGGTATGCATGTATATCCTCGGAGAAAGCATGAGCAGTATTAGGTCGAAA
TGCCCCGTCGAAGAGTCGGAAGCCTCCACACCACCTAGCACGCTGCCTTGCTTGTGCATCCATGCCA
TGACTCCAGAAAGAGTACAGCGCCTAAAAGCCTCACGTCCAGAACAAATTACTGTGTGCTCATCCTT
TCCATTGCCGAAGTATAGAATCACTGGTGTGCAGAAGATCCAATGCTCCCAGCCTATATTGTTCTCA
CCGAAAGTGCCTGCGTATATTCATCCAAGGAAGTATCTCGTGGAAACACCACCGGTAGACGAGACT
CCGGAGCCATCGGCAGAGAACCAATCCACAGAGGGGACACCTGAACAACCACCACTTATAACCGAG
GATGAGACCAGGACTAGAACGCCTGAGCCGATCATCATCGAAGAGGAAGAAGAGGATAGCATAAG
TTTGCTGTCAGATGGCCCGACCCACCAGGTGCTGCAAGTCGAGGCAGACATTCACGGGCCGCCCTCT
GTATCTAGCTCATCCTGGTCCATTCCTCATGCATCCGACTTTGATGTGGACAGTTTATCCATACTTGA
CACCCTGGAGGGAGCTAGCGTGACCAGCGGGGCAACGTCAGCCGAGACTAACTCTTACTTCGCAAA
GAGTATGGAGTTTCTGGCGCGACCGGTGCCTGCGCCTCGAACAGTATTCAGGAACCCTCCACATCCC
GCTCCGCGCACAAGAACACCGTCACTTGCACCCAGCAGGGCCTGCTCGAGAACCAGCCTAGTTTCC
ACCCCGCCAGGCGTGAATAGGGTGATCACTAGAGAGGAGCTCGAGGCGCTTACCCCGTCACGCACT
CCTAGCAGGTCGGTCTCGAGAACCAGCCTGGTCTCCAACCCGCCAGGCGTAAATAGGGTGATTACA
AGAGAGGAGTTTGAGGCGTTCGTAGCACAACAACAATGACGGTTTGATGCGGGTGCATACATCTTTT
CCTCCGACACCGGTCAAGGGCATTTACAACAAAAATCAGTAAGGCAAACGGTGCTATCCGAAGTGG
TGTTGGAGAGGACCGAATTGGAGATTTCGTATGCCCCGCGCCTCGACCAAGAAAAAGAAGAATTAC
TACGCAAGAAATTACAGTTAAATCCCACACCTGCTAACAGAAGCAGATACCAGTCCAGGAAGGTGG
AGAACATGAAAGCCATAACAGCTAGACGTATTCTGCAAGGCCTAGGGCATTATTTGAAGGCAGAAG
GAAAAGTGGAGTGCTACCGAACCCTGCATCCTGTTCCTTTGTATTCATCTAGTGTGAACCGTGCCTTT
TCAAGCCCCAAGGTCGCAGTGGAAGCCTGTAACGCCATGTTGAAAGAGAACTTTCCGACTGTGGCTT
CTTACTGTATTATTCCAGAGTACGATGCCTATTTGGACATGGTTGACGGAGCTTCATGCTGCTTAGAC
ACTGCCAGTTTTTGCCCTGCAAAGCTGCGCAGCTTTCCAAAGAAACACTCCTATTTGGAACCCACAA
TACGATCGGCAGTGCCTTCAGCGATCCAGAACACGCTCCAGAACGTCCTGGCAGCTGCCACAAAAA
GAAATTGCAATGTCACGCAAATGAGAGAATTGCCCGTATTGGATTCGGCGGCCTTTAATGTGGAATG
CTTCAAGAAATATGCGTGTAATAATGAATATTGGGAAACGTTTAAAGAAAACCCCATCAGGCTTACT
GAAGAAAACGTGGTAAATTACATTACCAAATTAAAAGGACCAAAAGCTGCTGCTCTTTTTGCGAAG
ACACATAATTTGAATATGTTGCAGGACATACCAATGGACAGGTTTGTAATGGACTTAAAGAGAGAC
GTGAAAGTGACTCCAGGAACAAAACATACTGAAGAACGGCCCAAGGTACAGGTGATCCAGGCTGCC
GATCCGCTAGCAACAGCGTATCTGTGCGGAATCCACCGAGAGCTGGTTAGGAGATTAAATGCGGTC
CTGCTTCCGAACATTCATACACTGTTTGATATGTCGGCTGAAGACTTTGACGCTATTATAGCCGAGC
ACTTCCAGCCTGGGGATTGTGTTCTGGAAACTGACATCGCGTCGTTTGATAAAAGTGAGGACGACGC
CATGGCTCTGACCGCGTTAATGATTCTGGAAGACTTAGGTGTGGACGCAGAGCTGTTGACGCTGATT
GAGGCGGCTTTCGGCGAAATTTCATCAATACATTTGCCCACTAAAACTAAATTTAAATTCGGAGCCA
TGATGAAATCTGGAATGTTCCTCACACTGTTTGTGAACACAGTCATTAACATTGTAATCGCAAGCAG
AGTGTTGAGAGAACGGCTAACCGGATCACCATGTGCAGCATTCATTGGAGATGACAATATCGTGAA
AGGAGTCAAATCGGACAAATTAATGGCAGACAGGTGCGCCACCTGGTTGAATATGGAAGTCAAGAT
TATAGATGCTGTGGTGGGCGAGAAAGCGCCTTATTTCTGTGGAGGGTTTATTTTGTGTGACTCCGTG
ACCGGCACAGCGTGCCGTGTGGCAGACCCCCTAAAAAGGCTGTTTAAGCTTGGCAAACCTCTGGCA
GCAGACGATGAACATGATGATGACAGGAGAAGGGCATTGCATGAAGAGTCAACACGCTGGAACCG
AGTGGGTATTCTTTCAGAGCTGTGCAAGGCAGTAGAATCAAGGTATGAAACCGTAGGAACTTCCAT
CATAGTTATGGCCATGACTACTCTAGCTAGCAGTGTTAAATCATTCAGCTACCTGAGAGGGGCCCCT
ATAACTCTCTACGGCTAACCTGAATGGACTACGACGTATCACGCCCAAACATTTACAGCCGCGGTGT
CAAAAACCGCGTGGACGTGGTTAACATCCCTGCTGGGAGGATCAGCCGTAATTATTATAATTGGCTT
GGTGCTGGCTACTATTGTGGCCATGTACGTGCTGACCAACCAGAAACATAATTGAATACAGCAGCA
ATTGGCAAGCTGCTTACATAGAACTCGCGGCGATTGGCATGCCGCCTTAAAATTTTTATTTTATTTTT
CTTTTCTTTTCCGAATCGGATTTTGTTTTTAATATTTCAAAAAAAAAAAAAAAAAAAAAAAAAAAAA
AAAAAAAAAAAAAAAAAAAAAAAAAAAAAAAAAAAAAAAAAAAAAAAAAAA
VEE Production Vector (SEQ ID NO: 8); VEE genome with nucleotides 7544-11175 deleted, plus 5′
T7-promoter, plus 3′ restriction sites
TAATACGACTCACTATAGGATGggcggcgcatgagagaagcccagaccaattacctacccaaaATGGagaaagttcacgttgacatcgag
gaagacagcccattcctcagagctttgcagcggagcttcccgcagtttgaggtagaagccaagcaggtcactgataatgaccatgctaatgccag
agcgttttcgcatctggcttcaaaactgatcgaaacggaggtggacccatccgacacgatccttgacattggaagtgcgcccgcccgcagaatgt
attctaagcacaagtatcattgtatctgtccgatgagatgtgcggaagatccggacagattgtataagtatgcaactaagctgaagaaaaactgt
aaggaaataactgataaggaattggacaagaaaatgaaggagctcgccgccgtcatgagcgaccctgacctggaaactgagactatgtgcctcca
cgacgacgagtcgtgtcgctacgaagggcaagtcgctgtttaccaggatgtatacgcggttgacggaccgacaagtctctatcaccaagccaata
agggagttagagtcgcctactggataggctttgacaccaccccttttatgtttaagaacttggctggagcatatccatcatactctaccaactgg
gccgacgaaaccgtgttaacggctcgtaacataggcctatgcagctctgacgttatggagcggtcacgtagagggatgtccattcttagaaagaa
gtatttgaaaccatccaacaatgttctattctctgttggctcgaccatctaccacgagaagagggacttactgaggagctggcacctgccgtctg
tatttcacttacgtggcaagcaaaattacacatgtcggtgtgagactatagttagttgcgacgggtacgtcgttaaaagaatagctatcagtcca
ggcctgtatgggaagccttcaggctatgctgctacgatgcaccgcgagggattcttgtgctgcaaagtgacagacacattgaacggggagagggt
ctcttttcccgtgtgcacgtatgtgccagctacattgtgtgaccaaatgactggcatactggcaacagatgtcagtgcggacgacgcgcaaaaac
tgctggttgggctcaaccagcgtatagtcgtcaacggtcgcacccagagaaacaccaataccatgaaaaattaccttttgcccgtagtggcccag
gcatttgctaggtgggcaaaggaatataaggaagatcaagaagatgaaaggccactaggactacgagatagacagttagtcatggggtgttgttg
ggcttttagaaggcacaagataacatctatttataagcgcccggatacccaaaccatcatcaaagtgaacagcgatttccactcattcgtgctgc
ccaggataggcagtaacacattggagatcgggctgagaacaagaatcaggaaaatgttagaggagcacaaggagccgtcacctctcattaccgcc
gaggacgtacaagaagctaagtgcgcagccgatgaggctaaggaggtgcgtgaagccgaggagttgcgcgcagctctaccacctttggcagctga
tgttgaggagcccactctggaagccgatgtcgacttgatgttacaagaggctggggccggctcagtggagacacctcgtggcttgataaaggtta
ccagctacgctggcgaggacaagatcggctcttacgctgtgctttctccgcaggctgtactcaagagtgaaaaattatcttgcatccaccctctc
gctgaacaagtcatagtgataacacactctggccgaaaagggcgttatgccgtggaaccataccatggtaaagtagtggtgccagagggacatgc
aatacccgtccaggactttcaagctctgagtgaaagtgccaccattgtgtacaacgaacgtgagttcgtaaacaggtacctgcaccatattgcca
cacatggaggagcgctgaacactgatgaagaatattacaaaactgtcaagcccagcgagcacgacggcgaatacctgtacgacatcgacaggaaa
cagtgcgtcaagaaagaactagtcactgggctagggctcacaggcgagctggtggatcctcccttccatgaattcgcctacgagagtctgagaac
acgaccagccgctccttaccaagtaccaaccataggggtgtatggcgtgccaggatcaggcaagtctggcatcattaaaagcgcagtcaccaaaa
aagatctagtggtgagcgccaagaaagaaaactgtgcagaaattataagggacgtcaagaaaatgaaagggctggacgtcaatgccagaactgtg
gactcagtgctcttgaatggatgcaaacaccccgtagagaccctgtatattgacgaagcttttgcttgtcatgcaggtactctcagagcgctcat
agccattataagacctaaaaaggcagtgctctgcggggatcccaaacagtgcggtttttttaacatgatgtgcctgaaagtgcattttaaccacg
agatttgcacacaagtcttccacaaaagcatctctcgccgttgcactaaatctgtgacttcggtcgtctcaaccttgttttacgacaaaaaaatg
agaacgacgaatccgaaagagactaagattgtgattgacactaccggcagtaccaaacctaagcaggacgatctcattctcacttgtttcagagg
ggggtgaagcagttgcaaatagattacaaaggcaacgaaataatgacggcagctgcctctcaagggctgacccgtaaaggtgtgtatgccgttcg
gtacaaggtgaatgaaaatcctctgtacgcacccacctcagaacatgtgaacgtcctactgacccgcacggaggaccgcatcgtgtggaaaacac
tagccggcgacccatggataaaaacactgactgccaagtaccctgggaatttcactgccacgatagaggagtggcaagcagagcatgatgccatc
atgaggcacatcttggagagaccggaccctaccgacgtcttccagaataaggcaaacgtgtgttgggccaaggctttagtgccggtgctgaagac
cgctggcatagacatgaccactgaacaatggaacactgtggattattttgaaacggacaaagctcactcagcagagatagtattgaaccaactat
gcgtgaggttctttggactcgatctggactccggtctattttctgcacccactgttccgttatccattaggaataatcactgggataactccccg
tcgcctaacatgtacgggctgaataaagaagtggtccgtcagctctctcgcaggtacccacaactgcctcgggcagttgccactggaagagtcta
tgacatgaacactggtacactgcgcaattatgatccgcgcataaacctagtacctgtaaacagaagactgcctcatgctttagtcctccaccata
atgaacacccacagagtgacttttcttcattcgtcagcaaattgaagggcagaactgtcctggtggtcggggaaaagttgtccgtcccaggcaaa
atggttgactggttgtcagaccggcctgaggctaccttcagagctcggctggatttaggcatcccaggtgatgtgcccaaatatgacataatatt
tgttaatgtgaggaccccatataaataccatcactatcagcagtgtgaagaccatgccattaagcttagcatgttgaccaagaaagcttgtctgc
atctgaatcccggcggaacctgtgtcagcataggttatggttacgctgacagggccagcgaaagcatcattggtgctatagcgcggcagttcaag
ttttcccgggtatgcaaaccgaaatcctcacttgaagagacggaagttctgtttgtattcattgggtacgatcgcaaggcccgtacgcacaatcc
ttacaagctttcatcaaccttgaccaacatttatacaggttccagactccacgaagccggatgtgcaccctcatatcatgtggtgcgaggggata
ttgccacggccaccgaaggagtgattataaatgctgctaacagcaaaggacaacctggcggaggggtgtgcggagcgctgtataagaaattcccg
gaaagcttcgatttacagccgatcgaagtaggaaaagcgcgactggtcaaaggtgcagctaaacatatcattcatgccgtaggaccaaacttcaa
caaagtttcggaggttgaaggtgacaaacagttggcagaggcttatgagtccatcgctaagattgtcaacgataacaattacaagtcagtagcga
ttccactgttgtccaccggcatcttttccgggaacaaagatcgactaacccaatcattgaaccatttgctgacagctttagacaccactgatgca
gatgtagccatatactgcagggacaagaaatgggaaatgactctcaaggaagcagtggctaggagagaagcagtggaggagatatgcatatccga
cgactcttcagtgacagaacctgatgcagagctggtgagggtgcatccgaagagttctttggctggaaggaagggctacagcacaagcgatggca
aaactttctcatatttggaagggaccaagtttcaccaggcggccaaggatatagcagaaattaatgccatgtggcccgttgcaacggaggccaat
gagcaggtatgcatgtatatcctcggagaaagcatgagcagtattaggtcgaaatgccccgtcgaagagtcggaagcctccacaccacctagcac
gctgccttgcttgtgcatccatgccatgactccagaaagagtacagcgcctaaaagcctcacgtccagaacaaattactgtgtgctcatcctttc
cattgccgaagtatagaatcactggtgtgcagaagatccaatgctcccagcctatattgttctcaccgaaagtgcctgcgtatattcatccaagg
aagtatctcgtggaaacaccaccggtagacgagactccggagccatcggcagagaaccaatccacagaggggacacctgaacaaccaccacttat
aaccgaggatgagaccaggactagaacgcctgagccgatcatcatcgaagaggaagaagaggatagcataagtttgctgtcagatggcccgaccc
accaggtgctgcaagtcgaggcagacattcacgggccgccctctgtatctagctcatcctggtccattcctcatgcatccgactttgatgtggac
agtttatccatacttgacaccctggagggagctagcgtgaccagcggggcaacgtcagccgagactaactcttacttcgcaaagagtatggagtt
tctggcgcgaccggtgcctgcgcctcgaacagtattcaggaaccctccacatcccgctccgcgcacaagaacaccgtcacttgcacccagcaggg
cctgctcgagaaccagcctagtttccaccccgccaggcgtgaatagggtgatcactagagaggagctcgaggcgcttaccccgtcacgcactcct
agcaggtcggtctcgagaaccagcctggtctccaacccgccaggcgtaaatagggtgattacaagagaggagtttgaggcgttcgtagcacaaca
acaatgacggtttgatgcgggtgcatacatcttttcctccgacaccggtcaagggcatttacaacaaaaatcagtaaggcaaacggtgctatccg
aagtggtgttggagaggaccgaattggagatttcgtatgccccgcgcctcgaccaagaaaaagaagaattactacgcaagaaattacagttaaat
cccacacctgctaacagaagcagataccagtccaggaaggtggagaacatgaaagccataacagctagacgtattctgcaaggcctagggcatta
tttgaaggcagaaggaaaagtggagtgctaccgaaccctgcatcctgttcctttgtattcatctagtgtgaaccgtgccttttcaagccccaagg
tcgcagtggaagcctgtaacgccatgttgaaagagaactttccgactgtggcttcttactgtattattccagagtacgatgcctatttggacatg
gttgacggagcttcatgctgcttagacactgccagtttttgccctgcaaagctgcgcagctttccaaagaaacactcctatttggaacccacaat
acgatcggcagtgccttcagcgatccagaacacgctccagaacgtcctggcagctgccacaaaaagaaattgcaatgtcacgcaaatgagagaat
tgcccgtattggattcggcggcctttaatgtggaatgcttcaagaaatatgcgtgtaataatgaatattgggaaacgtttaaagaaaaccccatc
aggcttactgaagaaaacgtggtaaattacattaccaaattaaaaggaccaaaagctgctgctctttttgcgaagacacataatttgaatatgtt
gcaggacataccaatggacaggtttgtaatggacttaaagagagacgtgaaagtgactccaggaacaaaacatactgaagaacggcccaaggtac
aggtgatccaggctgccgatccgctagcaacagcgtatctgtgcggaatccaccgagagctggttaggagattaaatgcggtcctgcttccgaac
attcatacactgtttgatatgtcggctgaagactttgacgctattatagccgagcacttccagcctggggattgtgttctggaaactgacatcgc
gtcgtttgataaaagtgaggacgacgccatggctctgaccgcgttaatgattctggaagacttaggtgtggacgcagagctgttgacgctgattg
aggcggctttcggcgaaatttcatcaatacatttgcccactaaaactaaatttaaattcggagccatgatgaaatctggaatgttcctcacactg
tttgtgaacacagtcattaacattgtaatcgcaagcagagtgttgagagaacggctaaccggatcaccatgtgcagcattcattggagatgacaa
tatcgtgaaaggagtcaaatcggacaaattaatggcagacaggtgcgccacctggttgaatatggaagtcaagattatagatgctgtggtgggcg
agaaagcgccttatttctgtggagggtttattttgtgtgactccgtgaccggcacagcgtgccgtgtggcagaccccctaaaaaggctgtttaag
cttggcaaacctctggcagcagacgatgaacatgatgatgacaggagaagggcattgcatgaagagtcaacacgctggaaccgagtgggtattct
ttcagagctgtgcaaggcagtagaatcaaggtatgaaaccgtaggaacttccatcatagttatggccatgactactctagctagcagtgttaaat
cattcagctacctgagaggggcccctataactctctacggcTAAcctgaatggactacgacgtatcacgcccaaacatttacagccgcggtgtca
aaaaccgcgtggacgtggttaacatccctgctgggaggatcagccgtaattattataattggcttggtgctggctactattgtggccatgtacgt
gctgaccaaccagaaacataattgaatacagcagcaattggcaagctgcttacatagaactcgcggcgattggcatgccgccttaaaatttttat
tttattttttcttttcttttccgaatcggattttgtttttaatatttcAAAAAAAAAAAAAAAAAAAAAAAAAAAAAAAAAAAAAAAAAAAAAAA
AAAAAAAAAAAAAAAAAAAAAAAAAAAAAAAAAtacgtagtttaaac
TC-83 Production Vector (SEQ ID NO: 9); TC-83 genome with nucleotides 7544-11175 deleted, plus
5′ T7-promoter, plus 3′ restriction sites
TAATACGACTCACTATAGGATAGGCGGCGCATGAGAGAAGCCCAGACCAATTACCTACCCAAAATG
GAGAAAGTTCACGTTGACATCGAGGAAGACAGCCCATTCCTCAGAGCTTTGCAGCGGAGCTTCCCG
CAGTTTGAGGTAGAAGCCAAGCAGGTCACTGATAATGACCATGCTAATGCCAGAGCGTTTTCGCATC
TGGCTTCAAAACTGATCGAAACGGAGGTGGACCCATCCGACACGATCCTTGACATTGGAAGTGCGC
CCGCCCGCAGAATGTATTCTAAGCACAAGTATCATTGTATCTGTCCGATGAGATGTGCGGAAGATCC
GGACAGATTGTATAAGTATGCAACTAAGCTGAAGAAAAACTGTAAGGAAATAACTGATAAGGAATT
GGACAAGAAAATGAAGGAGCTCGCCGCCGTCATGAGCGACCCTGACCTGGAAACTGAGACTATGTG
CCTCCACGACGACGAGTCGTGTCGCTACGAAGGGCAAGTCGCTGTTTACCAGGATGTATACGCGGTT
GACGGACCGACAAGTCTCTATCACCAAGCCAATAAGGGAGTTAGAGTCGCCTACTGGATAGGCTTT
GACACCACCCCTTTTATGTTTAAGAACTTGGCTGGAGCATATCCATCATACTCTACCAACTGGGCCG
ACGAAACCGTGTTAACGGCTCGTAACATAGGCCTATGCAGCTCTGACGTTATGGAGCGGTCACGTA
GAGGGATGTCCATTCTTAGAAAGAAGTATTTGAAACCATCCAACAATGTTCTATTCTCTGTTGGCTC
GACCATCTACCACGAGAAGAGGGACTTACTGAGGAGCTGGCACCTGCCGTCTGTATTTCACTTACGT
GGCAAGCAAAATTACACATGTCGGTGTGAGACTATAGTTAGTTGCGACGGGTACGTCGTTAAAAGA
ATAGCTATCAGTCCAGGCCTGTATGGGAAGCCTTCAGGCTATGCTGCTACGATGCACCGCGAGGGAT
TCTTGTGCTGCAAAGTGACAGACACATTGAACGGGGAGAGGGTCTCTTTTCCCGTGTGCACGTATGT
GCCAGCTACATTGTGTGACCAAATGACTGGCATACTGGCAACAGATGTCAGTGCGGACGACGCGCA
AAAACTGCTGGTTGGGCTCAACCAGCGTATAGTCGTCAACGGTCGCACCCAGAGAAACACCAATAC
CATGAAAAATTACCTTTTGCCCGTAGTGGCCCAGGCATTTGCTAGGTGGGCAAAGGAATATAAGGA
AGATCAAGAAGATGAAAGGCCACTAGGACTACGAGATAGACAGTTAGTCATGGGGTGTTGTTGGGC
TTTTAGAAGGCACAAGATAACATCTATTTATAAGCGCCCGGATACCCAAACCATCATCAAAGTGAA
CAGCGATTTCCACTCATTCGTGCTGCCCAGGATAGGCAGTAACACATTGGAGATCGGGCTGAGAAC
AAGAATCAGGAAAATGTTAGAGGAGCACAAGGAGCCGTCACCTCTCATTACCGCCGAGGACGTACA
AGAAGCTAAGTGCGCAGCCGATGAGGCTAAGGAGGTGCGTGAAGCCGAGGAGTTGCGCGCAGCTCT
ACCACCTTTGGCAGCTGATGTTGAGGAGCCCACTCTGGAAGCCGATGTCGACTTGATGTTACAAGAG
GCTGGGGCCGGCTCAGTGGAGACACCTCGTGGCTTGATAAAGGTTACCAGCTACGATGGCGAGGAC
AAGATCGGCTCTTACGCTGTGCTTTCTCCGCAGGCTGTACTCAAGAGTGAAAAATTATCTTGCATCC
ACCCTCTCGCTGAACAAGTCATAGTGATAACACACTCTGGCCGAAAAGGGCGTTATGCCGTGGAAC
CATACCATGGTAAAGTAGTGGTGCCAGAGGGACATGCAATACCCGTCCAGGACTTTCAAGCTCTGA
GTGAAAGTGCCACCATTGTGTACAACGAACGTGAGTTCGTAAACAGGTACCTGCACCATATTGCCAC
ACATGGAGGAGCGCTGAACACTGATGAAGAATATTACAAAACTGTCAAGCCCAGCGAGCACGACG
GCGAATACCTGTACGACATCGACAGGAAACAGTGCGTCAAGAAAGAACTAGTCACTGGGCTAGGGC
TCACAGGCGAGCTGGTGGATCCTCCCTTCCATGAATTCGCCTACGAGAGTCTGAGAACACGACCAGC
CGCTCCTTACCAAGTACCAACCATAGGGGTGTATGGCGTGCCAGGATCAGGCAAGTCTGGCATCATT
AAAAGCGCAGTCACCAAAAAAGATCTAGTGGTGAGCGCCAAGAAAGAAAACTGTGCAGAAATTAT
AAGGGACGTCAAGAAAATGAAAGGGCTGGACGTCAATGCCAGAACTGTGGACTCAGTGCTCTTGAA
TGGATGCAAACACCCCGTAGAGACCCTGTATATTGACGAAGCTTTTGCTTGTCATGCAGGTACTCTC
AGAGCGCTCATAGCCATTATAAGACCTAAAAAGGCAGTGCTCTGCGGGGATCCCAAACAGTGCGGT
TTTTTTAACATGATGTGCCTGAAAGTGCATTTTAACCACGAGATTTGCACACAAGTCTTCCACAAAA
GCATCTCTCGCCGTTGCACTAAATCTGTGACTTCGGTCGTCTCAACCTTGTTTTACGACAAAAAAATG
AGAACGACGAATCCGAAAGAGACTAAGATTGTGATTGACACTACCGGCAGTACCAAACCTAAGCAG
GACGATCTCATTCTCACTTGTTTCAGAGGGTGGGTGAAGCAGTTGCAAATAGATTACAAAGGCAAC
GAAATAATGACGGCAGCTGCCTCTCAAGGGCTGACCCGTAAAGGTGTGTATGCCGTTCGGTACAAG
GTGAATGAAAATCCTCTGTACGCACCCACCTCAGAACATGTGAACGTCCTACTGACCCGCACGGAG
GACCGCATCGTGTGGAAAACACTAGCCGGCGACCCATGGATAAAAACACTGACTGCCAAGTACCCT
GGGAATTTCACTGCCACGATAGAGGAGTGGCAAGCAGAGCATGATGCCATCATGAGGCACATCTTG
GAGAGACCGGACCCTACCGACGTCTTCCAGAATAAGGCAAACGTGTGTTGGGCCAAGGCTTTAGTG
CCGGTGCTGAAGACCGCTGGCATAGACATGACCACTGAACAATGGAACACTGTGGATTATTTTGAA
ACGGACAAAGCTCACTCAGCAGAGATAGTATTGAACCAACTATGCGTGAGGTTCTTTGGACTCGATC
TGGACTCCGGTCTATTTTCTGCACCCACTGTTCCGTTATCCATTAGGAATAATCACTGGGATAACTCC
CCGTCGCCTAACATGTACGGGCTGAATAAAGAAGTGGTCCGTCAGCTCTCTCGCAGGTACCCACAAC
TGCCTCGGGCAGTTGCCACTGGAAGAGTCTATGACATGAACACTGGTACACTGCGCAATTATGATCC
GCGCATAAACCTAGTACCTGTAAACAGAAGACTGCCTCATGCTTTAGTCCTCCACCATAATGAACAC
CCACAGAGTGACTTTTCTTCATTCGTCAGCAAATTGAAGGGCAGAACTGTCCTGGTGGTCGGGGAAA
AGTTGTCCGTCCCAGGCAAAATGGTTGACTGGTTGTCAGACCGGCCTGAGGCTACCTTCAGAGCTCG
GCTGGATTTAGGCATCCCAGGTGATGTGCCCAAATATGACATAATATTTGTTAATGTGAGGACCCCA
TATAAATACCATCACTATCAGCAGTGTGAAGACCATGCCATTAAGCTTAGCATGTTGACCAAGAAA
GCTTGTCTGCATCTGAATCCCGGCGGAACCTGTGTCAGCATAGGTTATGGTTACGCTGACAGGGCCA
GCGAAAGCATCATTGGTGCTATAGCGCGGCAGTTCAAGTTTTCCCGGGTATGCAAACCGAAATCCTC
ACTTGAAGAGACGGAAGTTCTGTTTGTATTCATTGGGTACGATCGCAAGGCCCGTACGCACAATCCT
TACAAGCTTTCATCAACCTTGACCAACATTTATACAGGTTCCAGACTCCACGAAGCCGGATGTGCAC
CCTCATATCATGTGGTGCGAGGGGATATTGCCACGGCCACCGAAGGAGTGATTATAAATGCTGCTA
ACAGCAAAGGACAACCTGGCGGAGGGGTGTGCGGAGCGCTGTATAAGAAATTCCCGGAAAGCTTCG
ATTTACAGCCGATCGAAGTAGGAAAAGCGCGACTGGTCAAAGGTGCAGCTAAACATATCATTCATG
CCGTAGGACCAAACTTCAACAAAGTTTCGGAGGTTGAAGGTGACAAACAGTTGGCAGAGGCTTATG
AGTCCATCGCTAAGATTGTCAACGATAACAATTACAAGTCAGTAGCGATTCCACTGTTGTCCACCGG
CATCTTTTCCGGGAACAAAGATCGACTAACCCAATCATTGAACCATTTGCTGACAGCTTTAGACACC
ACTGATGCAGATGTAGCCATATACTGCAGGGACAAGAAATGGGAAATGACTCTCAAGGAAGCAGTG
GCTAGGAGAGAAGCAGTGGAGGAGATATGCATATCCGACGACTCTTCAGTGACAGAACCTGATGCA
GAGCTGGTGAGGGTGCATCCGAAGAGTTCTTTGGCTGGAAGGAAGGGCTACAGCACAAGCGATGGC
AAAACTTTCTCATATTTGGAAGGGACCAAGTTTCACCAGGCGGCCAAGGATATAGCAGAAATTAAT
GCCATGTGGCCCGTTGCAACGGAGGCCAATGAGCAGGTATGCATGTATATCCTCGGAGAAAGCATG
AGCAGTATTAGGTCGAAATGCCCCGTCGAAGAGTCGGAAGCCTCCACACCACCTAGCACGCTGCCT
TGCTTGTGCATCCATGCCATGACTCCAGAAAGAGTACAGCGCCTAAAAGCCTCACGTCCAGAACAA
ATTACTGTGTGCTCATCCTTTCCATTGCCGAAGTATAGAATCACTGGTGTGCAGAAGATCCAATGCT
CCCAGCCTATATTGTTCTCACCGAAAGTGCCTGCGTATATTCATCCAAGGAAGTATCTCGTGGAAAC
ACCACCGGTAGACGAGACTCCGGAGCCATCGGCAGAGAACCAATCCACAGAGGGGACACCTGAAC
AACCACCACTTATAACCGAGGATGAGACCAGGACTAGAACGCCTGAGCCGATCATCATCGAAGAGG
AAGAAGAGGATAGCATAAGTTTGCTGTCAGATGGCCCGACCCACCAGGTGCTGCAAGTCGAGGCAG
ACATTCACGGGCCGCCCTCTGTATCTAGCTCATCCTGGTCCATTCCTCATGCATCCGACTTTGATGTG
GACAGTTTATCCATACTTGACACCCTGGAGGGAGCTAGCGTGACCAGCGGGGCAACGTCAGCCGAG
ACTAACTCTTACTTCGCAAAGAGTATGGAGTTTCTGGCGCGACCGGTGCCTGCGCCTCGAACAGTAT
TCAGGAACCCTCCACATCCCGCTCCGCGCACAAGAACACCGTCACTTGCACCCAGCAGGGCCTGCTC
GAGAACCAGCCTAGTTTCCACCCCGCCAGGCGTGAATAGGGTGATCACTAGAGAGGAGCTCGAGGC
GCTTACCCCGTCACGCACTCCTAGCAGGTCGGTCTCGAGAACCAGCCTGGTCTCCAACCCGCCAGGC
GTAAATAGGGTGATTACAAGAGAGGAGTTTGAGGCGTTCGTAGCACAACAACAATGACGGTTTGAT
GCGGGTGCATACATCTTTTCCTCCGACACCGGTCAAGGGCATTTACAACAAAAATCAGTAAGGCAA
ACGGTGCTATCCGAAGTGGTGTTGGAGAGGACCGAATTGGAGATTTCGTATGCCCCGCGCCTCGACC
AAGAAAAAGAAGAATTACTACGCAAGAAATTACAGTTAAATCCCACACCTGCTAACAGAAGCAGAT
ACCAGTCCAGGAAGGTGGAGAACATGAAAGCCATAACAGCTAGACGTATTCTGCAAGGCCTAGGGC
ATTATTTGAAGGCAGAAGGAAAAGTGGAGTGCTACCGAACCCTGCATCCTGTTCCTTTGTATTCATC
TAGTGTGAACCGTGCCTTTTCAAGCCCCAAGGTCGCAGTGGAAGCCTGTAACGCCATGTTGAAAGA
GAACTTTCCGACTGTGGCTTCTTACTGTATTATTCCAGAGTACGATGCCTATTTGGACATGGTTGACG
GAGCTTCATGCTGCTTAGACACTGCCAGTTTTTGCCCTGCAAAGCTGCGCAGCTTTCCAAAGAAACA
CTCCTATTTGGAACCCACAATACGATCGGCAGTGCCTTCAGCGATCCAGAACACGCTCCAGAACGTC
CTGGCAGCTGCCACAAAAAGAAATTGCAATGTCACGCAAATGAGAGAATTGCCCGTATTGGATTCG
GCGGCCTTTAATGTGGAATGCTTCAAGAAATATGCGTGTAATAATGAATATTGGGAAACGTTTAAAG
AAAACCCCATCAGGCTTACTGAAGAAAACGTGGTAAATTACATTACCAAATTAAAAGGACCAAAAG
CTGCTGCTCTTTTTGCGAAGACACATAATTTGAATATGTTGCAGGACATACCAATGGACAGGTTTGT
AATGGACTTAAAGAGAGACGTGAAAGTGACTCCAGGAACAAAACATACTGAAGAACGGCCCAAGG
TACAGGTGATCCAGGCTGCCGATCCGCTAGCAACAGCGTATCTGTGCGGAATCCACCGAGAGCTGG
TTAGGAGATTAAATGCGGTCCTGCTTCCGAACATTCATACACTGTTTGATATGTCGGCTGAAGACTT
TGACGCTATTATAGCCGAGCACTTCCAGCCTGGGGATTGTGTTCTGGAAACTGACATCGCGTCGTTT
GATAAAAGTGAGGACGACGCCATGGCTCTGACCGCGTTAATGATTCTGGAAGACTTAGGTGTGGAC
GCAGAGCTGTTGACGCTGATTGAGGCGGCTTTCGGCGAAATTTCATCAATACATTTGCCCACTAAAA
CTAAATTTAAATTCGGAGCCATGATGAAATCTGGAATGTTCCTCACACTGTTTGTGAACACAGTCAT
TAACATTGTAATCGCAAGCAGAGTGTTGAGAGAACGGCTAACCGGATCACCATGTGCAGCATTCAT
TGGAGATGACAATATCGTGAAAGGAGTCAAATCGGACAAATTAATGGCAGACAGGTGCGCCACCTG
GTTGAATATGGAAGTCAAGATTATAGATGCTGTGGTGGGCGAGAAAGCGCCTTATTTCTGTGGAGG
GTTTATTTTGTGTGACTCCGTGACCGGCACAGCGTGCCGTGTGGCAGACCCCCTAAAAAGGCTGTTT
AAGCTTGGCAAACCTCTGGCAGCAGACGATGAACATGATGATGACAGGAGAAGGGCATTGCATGAA
GAGTCAACACGCTGGAACCGAGTGGGTATTCTTTCAGAGCTGTGCAAGGCAGTAGAATCAAGGTAT
GAAACCGTAGGAACTTCCATCATAGTTATGGCCATGACTACTCTAGCTAGCAGTGTTAAATCATTCA
GCTACCTGAGAGGGGCCCCTATAACTCTCTACGGCTAACCTGAATGGACTACGACGTATCACGCCCA
AACATTTACAGCCGCGGTGTCAAAAACCGCGTGGACGTGGTTAACATCCCTGCTGGGAGGATCAGC
CGTAATTATTATAATTGGCTTGGTGCTGGCTACTATTGTGGCCATGTACGTGCTGACCAACCAGAAA
CATAATTGAATACAGCAGCAATTGGCAAGCTGCTTACATAGAACTCGCGGCGATTGGCATGCCGCCT
TAAAATTTTTATTTTATTTTTCTTTTCTTTTCCGAATCGGATTTTGTTTTTAATATTTCAAAAAAAAAA
AAAAAAAAAAAAAAAAAAAAAAAAAAAAAAAAAAAAAAAAAAAAAAAAAAAAAAAAAAAAAAA
AAAAAAAtacgtagtttaaac
VEE-UbAAY (SEQ ID NO: 14); VEE delivery vector with MHC class I mouse tumor epitopes SIINFEKL
and AH1-A5 inserted
VEE-Luciferase (SEQ ID NO: 15); VEE delivery vector with luciferase gene inserted at 7545
ubiquitin (SEQ ID NO: 38) >UbG76 0-228
Ubiquitin A76 (SEQ ID NO: 39) >UbA76 0-228
HLA-A2 (MHC class I) signal peptide (SEQ ID NO: 40) >MHC SignalPep 0-78
HLA-A2 (MHC class I) Trans Membrane domain (SEQ ID NO: 41) >HLA A2 TM Domain 0-201
IgK Leader Seq (SEQ ID NO: 42) >IgK Leader Seq 0-60
Human DC-Lamp (SEQ ID NO: 43) >HumanDCLAMP 0-3178
Mouse LAMP1 (SEQ ID NO: 44) >MouseLamp1 0-1858
Human Lamp1 cDNA (SEQ ID NO: 45) >Human Lamp1 0-2339
Tetanus toxoid nulceic acid sequence (SEQ ID NO: 46)
Tetanus toxoid amino acid sequence (SEQ ID NO: 47)
PADRE nulceotide sequence (SEQ ID NO: 48)
PADRE amino acid sequence (SEQ ID NO: 49)
WPRE (SEQ ID NO: 50) >WPRE 0-593
IRES (SEQ ID NO: 51) >eGFP_IRES_SEAP_Insert 1746-2335
GFP (SEQ ID NO: 52)
SEAP (SEQ ID NO: 53)
Firefly Luciferase (SEQ ID NO: 54)
FMDV 2A (SEQ ID NO: 55)
GPGPG linker (SEQ ID NO: 56)
SAM in vitro transcription template DNA (SEQ ID NO: 65); VEE genome with nucleotides 7544-11175
deleted, plus minimal 5′ T7-promoter (Bold Italic)
TAATACGACTCACTATAATGggcggcgcatgagagaagcccagaccaattacctacccaaaATGGagaaagttcacgttgacatcgaggaag
acagcccattcctcagagctttgcagcggagcttcccgcagtttgaggtagaagccaagcaggtcactgataatgaccatgctaatgccagagcg
ttttcgcatctggcttcaaaactgatcgaaacggaggtggacccatccgacacgatccttgacattggaagtgcgcccgcccgcagaatgtattc
taagcacaagtatcattgtatctgtccgatgagatgtgcggaagatccggacagattgtataagtatgcaactaagctgaagaaaaactgtaagg
aaataactgataaggaattggacaagaaaatgaaggagctcgccgccgtcatgagcgaccctgacctggaaactgagactatgtgcctccacgac
gacgagtcgtgtcgctacgaagggcaagtcgctgtttaccaggatgtatacgcggttgacggaccgacaagtctctatcaccaagccaataaggg
agttagagtcgcctactggataggctttgacaccaccccttttatgtttaagaacttggctggagcatatccatcatactctaccaactgggccg
acgaaaccgtgttaacggctcgtaacataggcctatgcagctctgacgttatggagcggtcacgtagagggatgtccattcttagaaagaagtat
ttgaaaccatccaacaatgttctattctctgttggctcgaccatctaccacgagaagagggacttactgaggagctggcacctgccgtctgtatt
tcacttacgtggcaagcaaaattacacatgtcggtgtgagactatagttagttgcgacgggtacgtcgttaaaagaatagctatcagtccaggcc
tgtatgggaagccttcaggctatgctgctacgatgcaccgcgagggattcttgtgctgcaaagtgacagacacattgaacggggagagggtctct
tttcccgtgtgcacgtatgtgccagctacattgtgtgaccaaatgactggcatactggcaacagatgtcagtgcggacgacgcgcaaaaactgct
ggttgggctcaaccagcgtatagtcgtcaacggtcgcacccagagaaacaccaataccatgaaaaattaccttttgcccgtagtggcccaggcat
ttgctagggggcaaaggaatataaggaagatcaagaagatgaaaggccactaggactacgagatagacagttagtcatggggtgttgttgggctt
ttagaaggcacaagataacatctatttataagcgcccggatacccaaaccatcatcaaagtgaacagcgatttccactcattcgtgctgcccagg
ataggcagtaacacattggagatcgggctgagaacaagaatcaggaaaatgttagaggagcacaaggagccgtcacctctcattaccgccgagga
cgtacaagaagctaagtgcgcagccgatgaggctaaggaggtgcgtgaagccgaggagttgcgcgcagctctaccacctttggcagctgatgttg
aggagcccactctggaagccgatgtcgacttgatgttacaagaggctggggccggctcagtggagacacctcgtggcttgataaaggttaccagc
tacgctggcgaggacaagatcggctcttacgctgtgctttctccgcaggctgtactcaagagtgaaaaattatcttgcatccaccctctcgctga
acaagtcatagtgataacacactctggccgaaaagggcgttatgccgtggaaccataccatggtaaagtagtggtgccagagggacatgcaatac
ccgtccaggactttcaagctctgagtgaaagtgccaccattgtgtacaacgaacgtgagttcgtaaacaggtacctgcaccatattgccacacat
ggaggagcgctgaacactgatgaagaatattacaaaactgtcaagcccagcgagcacgacggcgaatacctgtacgacatcgacaggaaacagtg
cgtcaagaaagaactagtcactgggctagggctcacaggcgagctggtggatcctcccttccatgaattcgcctacgagagtctgagaacacgac
cagccgctccttaccaagtaccaaccataggggtgtatggcgtgccaggatcaggcaagtctggcatcattaaaagcgcagtcaccaaaaaagat
ctagtggtgagcgccaagaaagaaaactgtgcagaaattataagggacgtcaagaaaatgaaagggctggacgtcaatgccagaactgtggactc
agtgctcttgaatggatgcaaacaccccgtagagaccctgtatattgacgaagcttttgcttgtcatgcaggtactctcagagcgctcatagcca
ttataagacctaaaaaggcagtgctctgcggggatcccaaacagtgcggtttttttaacatgatgtgcctgaaagtgcattttaaccacgagatt
tgcacacaagtcttccacaaaagcatctctcgccgttgcactaaatctgtgacttcggtcgtctcaaccttgttttacgacaaaaaaatgagaac
gacgaatccgaaagagactaagattgtgattgacactaccggcagtaccaaacctaagcaggacgatctcattctcacttgtttcagagggtggg
tgaagcagttgcaaatagattacaaaggcaacgaaataatgacggcagctgcctctcaagggctgacccgtaaaggtgtgtatgccgttcggtac
aaggtgaatgaaaatcctctgtacgcacccacctcagaacatgtgaacgtcctactgacccgcacggaggaccgcatcgtgtggaaaacactagc
cggcgacccatggataaaaacactgactgccaagtaccctgggaatttcactgccacgatagaggagtggcaagcagagcatgatgccatcatga
ggcacatcttggagagaccggaccctaccgacgtcttccagaataaggcaaacgtgtgttgggccaaggctttagtgccggtgctgaagaccgct
ggcatagacatgaccactgaacaatggaacactgtggattattttgaaacggacaaagctcactcagcagagatagtattgaaccaactatgcgt
gaggttctttggactcgatctggactccggtctattttctgcacccactgttccgttatccattaggaataatcactgggataactccccgtcgc
ctaacatgtacgggctgaataaagaagtggtccgtcagctctctcgcaggtacccacaactgcctcgggcagttgccactggaagagtctatgac
atgaacactggtacactgcgcaattatgatccgcgcataaacctagtacctgtaaacagaagactgcctcatgctttagtcctccaccataatga
acacccacagagtgacttttcttcattcgtcagcaaattgaagggcagaactgtcctggtggtcggggaaaagttgtccgtcccaggcaaaatgg
ttgactggttgtcagaccggcctgaggctaccttcagagctcggctggatttaggcatcccaggtgatgtgcccaaatatgacataatatttgtt
aatgtgaggaccccatataaataccatcactatcagcagtgtgaagaccatgccattaagcttagcatgttgaccaagaaagcttgtctgcatct
gaatcccggcggaacctgtgtcagcataggttatggttacgctgacagggccagcgaaagcatcattggtgctatagcgcggcagttcaagtttt
cccgggtatgcaaaccgaaatcctcacttgaagagacggaagttctgtttgtattcattgggtacgatcgcaaggcccgtacgcacaatccttac
aagctttcatcaaccttgaccaacatttatacaggttccagactccacgaagccggatgtgcaccctcatatcatgtggtgcgaggggatattgc
cacggccaccgaaggagtgattataaatgctgctaacagcaaaggacaacctggcggaggggtgtgcggagcgctgtataagaaattcccggaaa
gcttcgatttacagccgatcgaagtaggaaaagcgcgactggtcaaaggtgcagctaaacatatcattcatgccgtaggaccaaacttcaacaaa
gtttcggaggttgaaggtgacaaacagttggcagaggcttatgagtccatcgctaagattgtcaacgataacaattacaagtcagtagcgattcc
actgttgtccaccggcatcttttccgggaacaaagatcgactaacccaatcattgaaccatttgctgacagctttagacaccactgatgcagatg
tagccatatactgcagggacaagaaatgggaaatgactctcaaggaagcagtggctaggagagaagcagtggaggagatatgcatatccgacgac
tcttcagtgacagaacctgatgcagagctggtgagggtgcatccgaagagttctttggctggaaggaagggctacagcacaagcgatggcaaaac
tttctcatatttggaagggaccaagtttcaccaggggccaaggatatagcagaaattaatgccatgtggcccgttgcaacggaggccaatgagca
ggtatgcatgtatatcctcggagaaagcatgagcagtattaggtcgaaatgccccgtcgaagagtcggaagcctccacaccacctagcacgctgc
cttgcttgtgcatccatgccatgactccagaaagagtacagcgcctaaaagcctcacgtccagaacaaattactgtgtgctcatcctttccattg
ccgaagtatagaatcactggtgtgcagaagatccaatgctcccagcctatattgttctcaccgaaagtgcctgcgtatattcatccaaggaagta
tctcgtggaaacaccaccggtagacgagactccggagccatcggcagagaaccaatccacagaggggacacctgaacaaccaccacttataaccg
aggatgagaccaggactagaacgcctgagccgatcatcatcgaagaggaagaagaggatagcataagtttgctgtcagatggcccgacccaccag
gtgctgcaagtcgaggcagacattcacgggccgccctctgtatctagctcatcctggtccattcctcatgcatccgactttgatgtggacagttt
atccatacttgacaccctggagggagctagcgtgaccagcggggcaacgtcagccgagactaactcttacttcgcaaagagtatggagtttctgg
cgcgaccggtgcctgcgcctcgaacagtattcaggaaccctccacatcccgctccgcgcacaagaacaccgtcacttgcacccagcagggcctgc
tcgagaaccagcctagtttccaccccgccaggcgtgaatagggtgatcactagagaggagctcgaggcgcttaccccgtcacgcactcctagcag
gtcggtctcgagaaccagcctggtctccaacccgccaggcgtaaatagggtgattacaagagaggagtttgaggcgttcgtagcacaacaacaat
gacggtttgatgcgggtgcatacatcttttcctccgacaccggtcaagggcatttacaacaaaaatcagtaaggcaaacggtgctatccgaagtg
gtgttggagaggaccgaattggagatttcgtatgccccgcgcctcgaccaagaaaaagaagaattactacgcaagaaattacagttaaatcccac
acctgctaacagaagcagataccagtccaggaaggtggagaacatgaaagccataacagctagacgtattctgcaaggcctagggcattatttga
aggcagaaggaaaagtggagtgctaccgaaccctgcatcctgttcctttgtattcatctagtgtgaaccgtgccttttcaagccccaaggtcgca
gtggaagcctgtaacgccatgttgaaagagaactttccgactgtggcttcttactgtattattccagagtacgatgcctatttggacatggttga
cggagcttcatgctgcttagacactgccagtttttgccctgcaaagctgcgcagctttccaaagaaacactcctatttggaacccacaatacgat
cggcagtgccttcagcgatccagaacacgctccagaacgtcctggcagctgccacaaaaagaaattgcaatgtcacgcaaatgagagaattgccc
gtattggattcggcggcctttaatgtggaatgcttcaagaaatatgcgtgtaataatgaatattgggaaacgtttaaagaaaaccccatcaggct
tactgaagaaaacgtggtaaattacattaccaaattaaaaggaccaaaagctgctgctctttttgcgaagacacataatttgaatatgttgcagg
acataccaatggacaggtttgtaatggacttaaagagagacgtgaaagtgactccaggaacaaaacatactgaagaacggcccaaggtacaggtg
atccaggctgccgatccgctagcaacagcgtatctgtgcggaatccaccgagagctggttaggagattaaatgcggtcctgcttccgaacattca
tacactgtttgatatgtcggctgaagactttgacgctattatagccgagcacttccagcctggggattgtgttctggaaactgacatcgcgtcgt
ttgataaaagtgaggacgacgccatggctctgaccgcgttaatgattctggaagacttaggtgtggacgcagagctgttgacgctgattgaggcg
gctttcggcgaaatttcatcaatacatttgcccactaaaactaaatttaaattcggagccatgatgaaatctggaatgttcctcacactgtttgt
gaacacagtcattaacattgtaatcgcaagcagagtgttgagagaacggctaaccggatcaccatgtgcagcattcattggagatgacaatatcg
tgaaaggagtcaaatcggacaaattaatggcagacaggtgcgccacctggttgaatatggaagtcaagattatagatgctgtggtgggcgagaaa
gcgccttatttctgtggagggtttattttgtgtgactccgtgaccggcacagcgtgccgtgtggcagaccccctaaaaaggctgtttaagcttgg
caaacctctggcagcagacgatgaacatgatgatgacaggagaagggcattgcatgaagagtcaacacgctggaaccgagtgggtattctttcag
agctgtgcaaggcagtagaatcaaggtatgaaaccgtaggaacttccatcatagttatggccatgactactctagctagcagtgttaaatcattc
agctacctgagaggggcccctataactctctacggcTAAcctgaatggactacgactgTatcacgcccaaacatttacagccgcggtgtcaaaaa
ccgcgtggacgtggttaacatccctgctgggaggatcagccgtaattattataattggcttggtgctggctactattgtggccatgtacgtgctg
accaaccagaaacataattgaatacagcagcaattggcaagctgcttacatagaactcgcggcgattggcatgccgccttaaaatttttatttta
ttttttcttttcttttccgaatcggattttgtttttaatatttcAAAAAAAAAAAAAAAAAAAAAAAAAAAAAAAAAAAAAAAAAAAAAAAAAAA
AAAAAAAAAAAAAAAAAAAAAAAAAAAAA
19 bp TET operator (TETo) sequence (SEQ ID NO: 66)
TCCCTATCAGTGATAGAGA
CMT response region between I-Scel and AsisI sites of ChAdV68 vector backbone. The two
iterations of the 19 bp TETo sequences are bolded. The full-length CMV promoter is italicized
(SEQ ID NO: 67)
GACATTGATTATTGACTAGTTGTTAATAGTAATCAATTACGGGGTCATTAGTTCATAGCCCATATAT
GGAGTTCCGCGTTACATAACTTACGGTAAATGGCCCGCCTGGCTGACCGCCCAACGACCCCCGCCCATTG
ACGTCAATAATGACGTATGTTCCCATAGTAACGCCAATAGGGACTTTCCATTGACGTCAATGGGTGGAGTAT
TTACGGTAAACTGCCCACTTGGCAGTACATCAAGTGTATCATATGCCAAGTCCGCCCCCTATTGACGTCAAT
GACGGTAAATGGCCCGCCTGGCATTATGCCCAGTACATGACCTTACGGGACTTTCCTACTTGGCAGTACAT
CTACGTATTAGTCATCGCTATTACCATGGTGATGCGGTTTTGGCAGTACACCAATGGGCGTGGATAGCGGT
TTGACTCACGGGGATTTCCAAGTCTCCACCCCATTGACGTCAATGGGAGTTTGTTTTGGCACCAAAATCAAC
GGGACTTTCCAAAATGTCGTAATAACCCCGCCCCGTTGACGCAAATGGGCGGTAGGCGTGTACGGTGGGA
GGTCTATATAAGCAGAGCTCTCCCTATCAGTGATAGAGATCTCCCTATCAGTGATAGAGATC
ChAdV68-Empty-E4deleted; AC_000011.1 with E1 (nt 577 to 3403), E3 (nt 27,125- 31,825), and E4
region (nt 34,916 to 35,642) sequences deleted and the corresponding ATCC VR-594 (Independently
sequenced Full-Length VR-594 C68 SEQ ID NO: 10) nucleotides substituted at five positions

### REFERENCES

1. Desrichard, A., Snyder, A. & Chan, T. A. Cancer Neoantigens and Applications for Immunotherapy. Clin. Cancer Res. Off. J. Am. Assoc. Cancer Res. (2015). doi:10.1158/1078-0432.CCR-14-3175
2. Schumacher, T. N. & Schreiber, R. D. Neoantigens in cancer immunotherapy. Science 348, 69-74 (2015).
3. Gubin, M. M., Artyomov, M. N., Mardis, E. R. & Schreiber, R. D. Tumor neoantigens: building a framework for personalized cancer immunotherapy. J. Clin. Invest. 125, 3413-3421 (2015).
4. Rizvi, N. A. et al. Cancer immunology. Mutational landscape determines sensitivity to PD-1 blockade in non-small cell lung cancer. Science 348, 124-128 (2015).
5. Snyder, A. et al. Genetic basis for clinical response to CTLA-4 blockade in melanoma. N. Engl. J. Med. 371, 2189-2199 (2014).
6. Carreno, B. M. et al. Cancer immunotherapy. A dendritic cell vaccine increases the breadth and diversity of melanoma neoantigen-specific T cells. Science 348, 803-808 (2015).
7. Tran, E. et al. Cancer immunotherapy based on mutation-specific CD4+ T cells in a patient with epithelial cancer. Science 344, 641-645 (2014).
8. Hacohen, N. & Wu, C. J.-Y. United States Patent Application: 20110293637—COMPOSITIONS AND METHODS OF IDENTIFYING TUMOR SPECIFIC NEOANTIGENS. (A1). at <http://appftl.uspto.gov/netacgi/nph-Parser?Sect1=PTO1&Sect2=HITOFF&d=PG01&p=1&u=/netahtml/PTO/srchnum.html&r-1&f=G&l=50&s1=20110293637.PGNR.>
9. Lundegaard, C., Hoof, I., Lund, O. & Nielsen, M. State of the art and challenges in sequence based T-cell epitope prediction. Immunome Res. 6 Suppl 2, S3 (2010).
10. Yadav, M. et al. Predicting immunogenic tumour mutations by combining mass spectrometry and exome sequencing. Nature 515, 572-576 (2014).
11. Bassani-Sternberg, M., Pletscher-Frankild, S., Jensen, L. J. & Mann, M. Mass spectrometry of human leukocyte antigen class I peptidomes reveals strong effects of protein abundance and turnover on antigen presentation. Mol. Cell. Proteomics MCP 14, 658-673 (2015).
12. Van Allen, E. M. et al. Genomic correlates of response to CTLA-4 blockade in metastatic melanoma. Science 350, 207-211 (2015).
13. Yoshida, K. & Ogawa, S. Splicing factor mutations and cancer. Wiley Interdiscip. Rev. RNA 5, 445-459 (2014).
14. Cancer Genome Atlas Research Network. Comprehensive molecular profiling of lung adenocarcinoma. Nature 511, 543-550 (2014).
15. Rajasagi, M. et al. Systematic identification of personal tumor-specific neoantigens in chronic lymphocytic leukemia. Blood 124, 453-462 (2014).
16. Downing, S. R. et al. United States Patent Application: 0120208706—OPTIMIZATION OF MULTIGENE ANALYSIS OF TUMOR SAMPLES. (A1). at <http://appftl.uspto.gov/netacgi/nph-Parser?Sect1=PTO1&Sect2=HITOFF&d=PG01&p=1&u=/netahtml/PTO/srchnum.html&r-1&f=G&l=50&s1=20120208706.PGNR.>
17. Target Capture for NextGen Sequencing—IDT. at <http://www.idtdna.com/pages/products/nextgen/target-capture>
18. Shukla, S. A. et al. Comprehensive analysis of cancer-associated somatic mutations in class I HLA genes. Nat. Biotechnol. 33, 1152-1158 (2015).
19. Cieslik, M. et al. The use of exome capture RNA-seq for highly degraded RNA with application to clinical cancer sequencing. Genome Res. 25, 1372-1381 (2015).
20. Bodini, M. et al. The hidden genomic landscape of acute myeloid leukemia: subclonal structure revealed by undetected mutations. Blood 125, 600-605 (2015).
21. Saunders, C. T. et al. Strelka: accurate somatic small-variant calling from sequenced tumor-normal sample pairs. Bioinforma. Oxf. Engl. 28, 1811-1817 (2012).
22. Cibulskis, K. et al. Sensitive detection of somatic point mutations in impure and heterogeneous cancer samples. Nat. Biotechnol. 31, 213-219 (2013).
23. Wilkerson, M. D. et al. Integrated RNA and DNA sequencing improves mutation detection in low purity tumors. Nucleic Acids Res. 42, e107 (2014).
24. Mose, L. E., Wilkerson, M. D., Hayes, D. N., Perou, C. M. & Parker, J. S. ABRA: improved coding indel detection via assembly-based realignment. Bioinforma. Oxf Engl. 30, 2813-2815 (2014).
25. Ye, K., Schulz, M. H., Long, Q., Apweiler, R. & Ning, Z. Pindel: a pattern growth approach to detect break points of large deletions and medium sized insertions from paired-end short reads. Bioinforma. Oxf Engl. 25, 2865-2871 (2009).
26. Lam, H. Y. K. et al. Nucleotide-resolution analysis of structural variants using BreakSeq and a breakpoint library. Nat. Biotechnol. 28, 47-55 (2010).
27. Frampton, G. M. et al. Development and validation of a clinical cancer genomic profiling test based on massively parallel DNA sequencing. Nat. Biotechnol. 31, 1023-1031 (2013).
28. Boegel, S. et al. HLA typing from RNA-Seq sequence reads. Genome Med. 4, 102 (2012).
29. Liu, C. et al. ATHLATES: accurate typing of human leukocyte antigen through exome sequencing. Nucleic Acids Res. 41, e142 (2013).
30. Mayor, N. P. et al. HLA Typing for the Next Generation. PloS One 10, e0127153 (2015).
31. Roy, C. K., Olson, S., Graveley, B. R., Zamore, P. D. & Moore, M. J. Assessing long-distance RNA sequence connectivity via RNA-templated DNA-DNA ligation. eLife 4, (2015).
32. Song, L. & Florea, L. CLASS: constrained transcript assembly of RNA-seq reads. BMC Bioinformatics 14 Suppl 5, S14 (2013).
33. Maretty, L., Sibbesen, J. A. & Krogh, A. Bayesian transcriptome assembly. Genome Biol. 15, 501 (2014).
34. Pertea, M. et al. StringTie enables improved reconstruction of a transcriptome from RNA-seq reads. Nat. Biotechnol. 33, 290-295 (2015).
35. Roberts, A., Pimentel, H., Trapnell, C. & Pachter, L. Identification of novel transcripts in annotated genomes using RNA-Seq. Bioinforma. Oxf Engl. (2011). doi:10.1093/bioinformatics/btr355
36. Vitting-Seerup, K., Porse, B. T., Sandelin, A. & Waage, J. spliceR: an R package for classification of alternative splicing and prediction of coding potential from RNA-seq data. BMC Bioinformatics 15, 81 (2014).
37. Rivas, M. A. et al. Human genomics. Effect of predicted protein-truncating genetic variants on the human transcriptome. Science 348, 666-669 (2015).
38. Skelly, D. A., Johansson, M., Madeoy, J., Wakefield, J. & Akey, J. M. A powerful and flexible statistical framework for testing hypotheses of allele-specific gene expression from RNA-seq data. Genome Res. 21, 1728-1737 (2011).
39. Anders, S., Pyl, P. T. & Huber, W. HTSeq—a Python framework to work with high-throughput sequencing data. Bioinforma. Oxf Engl. 31, 166-169 (2015).
40. Furney, S. J. et al. SF3B1 mutations are associated with alternative splicing in uveal melanoma. Cancer Discov. (2013). doi:10.1158/2159-8290.CD-13-0330
41. Zhou, Q. et al. A chemical genetics approach for the functional assessment of novel cancer genes. Cancer Res. (2015). doi:10.1158/0008-5472.CAN-14-2930
42. Maguire, S. L. et al. SF3B1 mutations constitute a novel therapeutic target in breast cancer. J. Pathol. 235, 571-580 (2015).
43. Carithers, L. J. et al. A Novel Approach to High-Quality Postmortem Tissue Procurement: The GTEx Project. Biopreservation Biobanking 13, 311-319 (2015).
44. Xu, G. et al. RNA CoMPASS: a dual approach for pathogen and host transcriptome analysis of RNA-seq datasets. PloS One 9, e89445 (2014).
45. Andreatta, M. & Nielsen, M. Gapped sequence alignment using artificial neural networks: application to the MHC class I system. Bioinforma. Oxf Engl. (2015). doi:10.1093/bioinformatics/btv639
46. Jørgensen, K. W., Rasmussen, M., Buus, S. & Nielsen, M. NetMHCstab—predicting stability of peptide-MHC-I complexes; impacts for cytotoxic T lymphocyte epitope discovery. Immunology 141, 18-26 (2014).
47. Larsen, M. V. et al. An integrative approach to CTL epitope prediction: a combined algorithm integrating MHC class I binding, TAP transport efficiency, and proteasomal cleavage predictions. Eur. J. Immunol. 35, 2295-2303 (2005).
48. Nielsen, M., Lundegaard, C., Lund, O. & Ke mir, C. The role of the proteasome in generating cytotoxic T-cell epitopes: insights obtained from improved predictions of proteasomal cleavage. Immunogenetics 57, 33-41 (2005).
49. Boisvert, F.-M. et al. A Quantitative Spatial Proteomics Analysis of Proteome Turnover in Human Cells. Mol. Cell. Proteomics 11, M111.011429-M111.011429 (2012).
50. Duan, F. et al. Genomic and bioinformatic profiling of mutational neoepitopes reveals new rules to predict anticancer immunogenicity. J. Exp. Med. 211, 2231-2248 (2014).
51. Janeway's Immunobiology: 9780815345312: Medicine & Health Science Books @Amazon.com. at <http://www.amazon.com/Janeways-Immunobiology-Kenneth-Murphy/dp/0815345313>
52. Calis, J. J. A. et al. Properties of MHC Class I Presented Peptides That Enhance Immunogenicity. PLoS Comput. Biol. 9, e1003266 (2013).
53. Zhang, J. et al. Intratumor heterogeneity in localized lung adenocarcinomas delineated by multiregion sequencing. Science 346, 256-259 (2014)
54. Walter, M. J. et al. Clonal architecture of secondary acute myeloid leukemia. N. Engl. J. Med. 366, 1090-1098 (2012).
55. Hunt D F, Henderson R A, Shabanowitz J, Sakaguchi K, Michel H, Sevilir N, Cox A L, Appella E, Engelhard V H. Characterization of peptides bound to the class I MHC molecule HLA-A2.1 by mass spectrometry. Science 1992. 255: 1261-1263.
56. Zarling A L, Polefrone J M, Evans A M, Mikesh L M, Shabanowitz J, Lewis S T, Engelhard V H, Hunt D F. Identification of class I MHC-associated phosphopeptides as targets for cancer immunotherapy. Proc Natl Acad Sci USA. 2006 Oct. 3; 103(40):14889-94.
57. Bassani-Sternberg M, Pletscher-Frankild S, Jensen L J, Mann M. Mass spectrometry of human leukocyte antigen class I peptidomes reveals strong effects of protein abundance and turnover on antigen presentation. Mol Cell Proteomics. 2015 March; 14(3):658-73. doi: 10.1074/mcp.M114.042812.
58. Abelin J G, Trantham P D, Penny S A, Patterson A M, Ward S T, Hildebrand W H, Cobbold M, Bai D L, Shabanowitz J, Hunt D F. Complementary IMAC enrichment methods for HLA-associated phosphopeptide identification by mass spectrometry. Nat Protoc. 2015 September; 10(9):1308-18. doi: 10.1038/nprot.2015.086. Epub 2015 Aug. 6
59. Barnstable C J, Bodmer W F, Brown G, Galfre G, Milstein C, Williams A F, Ziegler A. Production of monoclonal antibodies to group A erythrocytes, HLA and other human cell surface antigens-new tools for genetic analysis. Cell. 1978 May; 14(1):9-20.
60. Goldman J M, Hibbin J, Kearney L, Orchard K, Th'ng K H. HLA-D R monoclonal antibodies inhibit the proliferation of normal and chronic granulocytic leukaemia myeloid progenitor cells. Br J Haematol. 1982 November; 52(3):411-20.
61. Eng J K, Jahan T A, Hoopmann M R. Comet: an open-source M S/M S sequence database search tool. Proteomics. 2013 January; 13(1):22-4. doi: 10.1002/pmic.201200439. Epub 2012 Dec. 4.
62. Eng J K, Hoopmann M R, Jahan T A, Egertson J D, Noble W S, MacCoss M J. A deeper look into Comet—implementation and features. J Am Soc Mass Spectrom. 2015 November; 26(11):1865-74. doi: 10.1007/s13361-015-1179-x. Epub 2015 Jun. 27.
63. Lukas Käll, Jesse Canterbury, Jason Weston, William Stafford Noble and Michael J. MacCoss. Semi-supervised learning for peptide identification from shotgun proteomics datasets. Nature Methods 4:923-925, November 2007
64. Lukas Käll, John D. Storey, Michael J. MacCoss and William Stafford Noble. Assigning confidence measures to peptides identified by tandem mass spectrometry. Journal of Proteome Research, 7(1):29-34, January 2008
65. Lukas Kall, John D. Storey and William Stafford Noble. Nonparametric estimation of posterior error probabilities associated with peptides identified by tandem mass spectrometry. Bioinformatics, 24(16):i42-i48, August 2008
66. Kinney R M, B J Johnson, V L Brown, D W Trent. Nucleotide Sequence of the 26 S mRNA of the Virulent Trinidad Donkey Strain of Venezuelan Equine Encephalitis Virus and Deduced Sequence of the Encoded Structural Proteins. Virology 152 (2), 400-413. 1986 Jul. 30.
67. Jill E Slansky, Frédérique M Rattis, Lisa F Boyd, Tarek Fahmy, Elizabeth M Jaffee, Jonathan P Schneck, David H Margulies, Drew M Pardoll. Enhanced Antigen-Specific Antitumor Immunity with Altered Peptide Ligands that Stabilize the MHC-Peptide-TCR Complex. Immunity, Volume 13, Issue 4, 1 Oct. 2000, Pages 529-538.
68. A Y Huang, P H Gulden, A S Woods, M C Thomas, C D Tong, W Wang, V H Engelhard, G Pasternack, R Cotter, D Hunt, D M Pardoll, and E M Jaffee. The immunodominant major histocompatibility complex class I-restricted antigen of a murine colon tumor derives from an endogenous retroviral gene product. Proc Natl Acad Sci USA.; 93(18): 9730-9735, 1996 Sep. 3.
69. JOHNSON, BARBARA J. B., RICHARD M. KINNEY, CRYSTLE L. KOST AND DENNIS W. TRENT. Molecular Determinants of Alphavirus Neurovirulence: Nucleotide and Deduced Protein Sequence Changes during Attenuation of Venezuelan Equine Encephalitis Virus. J Gen Virol 67:1951-1960, 1986.
70. Aarnoudse, C. A., Kruse, M., Konopitzky, R., Brouwenstijn, N., and Schrier, P. I. (2002). TCR reconstitution in Jurkat reporter cells facilitates the identification of novel tumor antigens by cDNA expression cloning. Int J Cancer 99, 7-13.
71. Alexander, J., Sidney, J., Southwood, S., Ruppert, J., Oseroff, C., Maewal, A., Snoke, K., Serra, H. M., Kubo, R. T., and Sette, A. (1994). Development of high potency universal DR-restricted helper epitopes by modification of high affinity DR-blocking peptides. Immunity 1, 751-761.
72. Banu, N., Chia, A., Ho, Z. Z., Garcia, A. T., Paravasivam, K., Grotenbreg, G. M., Bertoletti, A., and Gehring, A. J. (2014). Building and optimizing a virus-specific T cell receptor library for targeted immunotherapy in viral infections. Scientific Reports 4, 4166.
73. Cornet, S., Miconnet, I., Menez, J., Lemonnier, F., and Kosmatopoulos, K. (2006). Optimal organization of a polypeptide-based candidate cancer vaccine composed of cryptic tumor peptides with enhanced immunogenicity. Vaccine 24, 2102-2109.
74. Depla, E., van der Aa, A., Livingston, B. D., Crimi, C., Allosery, K., de Brabandere, V., Krakover, J., Murthy, S., Huang, M., Power, S., et al. (2008). Rational design of a multiepitope vaccine encoding T-lymphocyte epitopes for treatment of chronic hepatitis B virus infections. Journal of Virology 82, 435-450.
75. Ishioka, G. Y., Fikes, J., Hermanson, G., Livingston, B., Crimi, C., Qin, M., del Guercio, M. F., Oseroff, C., Dahlberg, C., Alexander, J., et al. (1999). Utilization of MHC class I transgenic mice for development of minigene DNA vaccines encoding multiple HLA-restricted CTL epitopes. J Immunol 162, 3915-3925.
76. Janetzki, S., Price, L., Schroeder, H., Britten, C. M., Welters, M. J. P., and Hoos, A. (2015). Guidelines for the automated evaluation of Elispot assays. Nat Protoc 10, 1098-1115.
77. Lyons, G. E., Moore, T., Brasic, N., Li, M., Roszkowski, J. J., and Nishimura, M. I. (2006). Influence of human CD8 on antigen recognition by T-cell receptor-transduced cells. Cancer Res 66, 11455-11461.
78. Nagai, K., Ochi, T., Fujiwara, H., An, J., Shirakata, T., Mineno, J., Kuzushima, K., Shiku, H., Melenhorst, J. J., Gostick, E., et al. (2012). Aurora kinase A-specific T-cell receptor gene transfer redirects T lymphocytes to display effective antileukemia reactivity. Blood 119, 368-376.
79. Panina-Bordignon, P., Tan, A., Termijtelen, A., Demotz, S., Corradin, G., and Lanzavecchia, A. (1989). Universally immunogenic T cell epitopes: promiscuous binding to human MHC class II and promiscuous recognition by T cells. Eur J Immunol 19, 2237-2242.
80. Vitiello, A., Marchesini, D., Furze, J., Sherman, L. A., and Chesnut, R. W. (1991). Analysis of the HLA-restricted influenza-specific cytotoxic T lymphocyte response in transgenic mice carrying a chimeric human-mouse class I major histocompatibility complex. J Exp Med 173, 1007-1015.
81. Yachi, P. P., Ampudia, J., Zal, T., and Gascoigne, N. R. J. (2006). Altered peptide ligands induce delayed CD8-T cell receptor interaction—a role for CD8 in distinguishing antigen quality. Immunity 25, 203-211.
82. Pushko P, Parker M, Ludwig G V, Davis N L, Johnston R E, Smith J F. Replicon-helper systems from attenuated Venezuelan equine encephalitis virus: expression of heterologous genes in vitro and immunization against heterologous pathogens in vivo. Virology. 1997 Dec. 22; 239(2):389-401.
83. Strauss, J H and E G Strauss. The alphaviruses: gene expression, replication, and evolution. Microbiol Rev. 1994 September; 58(3): 491-562.
84. Rhême C, Ehrengruber M U, Grandgirard D. Alphaviral cytotoxicity and its implication in vector development. Exp Physiol. 2005 January; 90(1):45-52. Epub 2004 Nov. 12.
85. Riley, Michael K. I I, and Wilfred Vermerris. Recent Advances in Nanomaterials for Gene Delivery-A Review. Nanomaterials 2017, 7(5), 94.
86. Frolov I, Hardy R, Rice C M. Cis-acting RNA elements at the 5′ end of Sindbis virus genome RNA regulate minus- and plus-strand RNA synthesis. RNA. 2001 November; 7(11):1638-51.
87. Jose J, Snyder J E, Kuhn R J. A structural and functional perspective of alphavirus replication and assembly. Future Microbiol. 2009 September; 4(7):837-56.
88. Bo Li and C. olin N. Dewey. RSEM: accurate transcript quantification from RNA-Seq data with or without a referenfe genome. BMC Bioinformatics, 12:323, August 2011
89. Hillary Pearson, Tariq Daouda, Diana Paola Granados, Chantal Durette, Eric Bonneil, Mathieu Courcelles, Anja Rodenbrock, Jean-Philippe Laverdure, Caroline Côté, Sylvie Mader, Sébastien Lemieux, Pierre Thibault, and Claude Perreault. MHC class I-associated peptides derive from selective regions of the human genome. The Journal of Clinical Investigation, 2016,
90. Juliane Liepe, Fabio Marino, John Sidney, Anita Jeko, Daniel E. Bunting, Alessandro Sette, Peter M. Kloetzel, Michael P. H. Stumpf, Albert J. R. Heck, Michele Mishto. A large fraction of HLA class I ligands are proteasome-generated spliced peptides. Science, 21, October 2016.
91. Mommen G P., Marino, F., Meiring H D., Poelen, M C., van Gaans-van den Brink, J A., Mohammed S., Heck A J., and van Els C A. Sampling From the Proteome to the Human Leukocyte Antigen-D R (HLA-D R) Ligandome Proceeds Via High Specificity. Mol Cell Proteomics 15(4): 1412-1423, April 2016.
92. Sebastian Kreiter, Mathias Vormehr, Niels van de Roemer, Mustafa Diken, Martin Löwer, Jan Diekmann, Sebastian Boegel, Barbara Schrörs, Fulvia Vascotto, John C. Castle, Arbel D. Tadmor, Stephen P. Schoenberger, Christoph Huber, Özlem Türeci, and Ugur Sahin. Mutant MHC class II epitopes drive therapeutic immune responses to caner. Nature 520, 692-696, April 2015.
93. Tran E., Turcotte S., Gros A., Robbins P. F., Lu Y. C., Dudley M. E., Wunderlich J. R., Somerville R. P., Hogan K., Hinrichs C. S., Parkhurst M. R., Yang J. C., Rosenberg S. A. Cancer immunotherapy based on mutation-specific CD4+ T cells in a patient with epithelial cancer. Science 344(6184) 641-645, May 2014.
94. Andreatta M., Karosiene E., Rasmussen M., Stryhn A., Buus S., Nielsen M. Accurate pan-specific prediction of peptide-MHC class II binding affinity with improved binding core identification. Immunogenetics 67(11-12) 641-650, November 2015.
95. Nielsen, M., Lund, O. N N-align. An artificial neural network-based alignment algorithm for MHC class II peptide binding prediction. BMC Bioinformatics 10:296, September 2009.
96. Nielsen, M., Lundegaard, C., Lund, O. Prediction of MHC class II binding affinity using SMM-align, a novel stabilization matrix alignment method. BMC Bioinformatics 8:238, July 2007.
97. Zhang, J., et al. PEAKS DB: de novo sequencing assisted database search for sensitive and accurate peptide identification. Molecular & Cellular Proteomics. 11(4):1-8. 1/2/2012.
98. Jensen, Kamilla Kjaergaard, et al. “Improved Methods for Prediting Peptide Binding Affinity to MHC Class II Molecules.” Immunology, 2018, doi:10.1111/imm.12889.
99. Carter, S. L., Cibulskis, K., Helman, E., McKenna, A., Shen, H., Zack, T., Laird, P. W., Onofrio, R. C., Winckler, W., Weir, B. A., et al. (2012). Absolute quantification of somatic DNA alterations in human cancer. Nat. Biotechnol. 30, 413-421
100. McGranahan, N., Rosenthal, R., Hiley, C. T., Rowan, A. J., Watkins, T. B. K., Wilson, G. A., Birkbak, N. J., Veeriah, S., Van Loo, P., Herrero, J., et al. (2017). Allele-Specific HLA Loss and Immune Escape in Lung Cancer Evolution. Cell 171, 1259-1271.e11.
101. Shukla, S. A., Rooney, M. S., Rajasagi, M., Tiao, G., Dixon, P. M., Lawrence, M. S., Stevens, J., Lane, W. J., Dellagatta, J. L., Steelman, S., et al. (2015). Comprehensive analysis of cancer-associated somatic mutations in class I HLA genes. Nat. Biotechnol. 33, 1152-1158.
102. Van Loo, P., Nordgard, S. H., Lingjærde, O. C., Russnes, H. G., Rye, I. H., Sun, W., Weigman, V. J., Marynen, P., Zetterberg, A., Naume, B., et al. (2010). Allele-specific copy number analysis of tumors. Proc. Natl. Acad. Sci. U.S.A 107, 16910-16915.
103. Van Loo, P., Nordgard, S. H., Lingjærde, O. C., Russnes, H. G., Rye, I. H., Sun, W., Weigman, V. J., Marynen, P., Zetterberg, A., Naume, B., et al. (2010). Allele-specific copy number analysis of tumors. Proc. Natl. Acad. Sci. U.S.A 107, 16910-16915.
104. MacLean, B., Tomazela, D. M., Shulman, N., Chambers, M., Finney, G. L., Frewen, B., Kern, R., Tabb, D. L., Liebler, D. C., MacCoss, M. J., (2010). Skyline: an open source document editor for creating and analyzing targeted proteomics experiments. Bioinformatics 26(7), 966-8.

1. An antigen-encoding vaccine system, wherein the antigen-encoding vaccine system comprises:
(a) a Cancer Testis Antigen (CTA)-encoding nucleic acid sequence encoding a CTA-associated MHC class I epitope; and
(b) a KRAS-encoding nucleic acid sequence encoding a KRAS-associated MHC class I epitope.

2.-5. (canceled)

6. The vaccine system of claim 1, wherein:
a) the CTA-encoding nucleic acid sequence comprises two or more distinct CTA-encoding nucleic acid sequences, wherein each distinct CTA-encoding nucleic acid sequence encodes a non-identical CTA-associated MHC class I epitope, optionally wherein the system comprises two or more iterations of the CTA-encoding nucleic acid sequence;
(b) the KRAS-encoding nucleic acid sequence comprises two or more distinct KRAS-encoding nucleic acid sequences, wherein each distinct KRAS-encoding nucleic acid sequence encodes a non-identical KRAS-associated MHC class I epitope, optionally wherein system comprises two or more iterations of the KRAS-encoding nucleic acid sequence; or
(c) the CTA-encoding nucleic acid sequence comprises two or more distinct CTA-encoding nucleic acid sequences, wherein each distinct CTA-encoding nucleic acid sequence encodes a non-identical CTA-associated MHC class I epitope and the KRAS-encoding nucleic acid sequence comprises two or more distinct KRAS-encoding nucleic acid sequences, wherein each distinct KRAS-encoding nucleic acid sequence encodes a non-identical KRAS-associated MHC class I epitope, optionally wherein the system comprises two or more iterations of each of the CTA-encoding nucleic acid sequence and the KRAS-encoding nucleic acid sequence, and wherein each iteration of the CTA-encoding nucleic acid sequence and/or the KRAS-encoding nucleic acid sequence, respectively, comprises identical nucleic acid sequences;
optionally wherein the two or more iterations comprises:
(i) at least 3, at least 4, at least 5, at least 6, at least 7 iterations, or at least 8 iterations;
(ii) at least 4 iterations;
(iii) at least 2 iterations of the CTA-encoding nucleic acid sequence and the KRAS-encoding nucleic acid sequence;
(iv) at least 2 iterations of the KRAS-encoding nucleic acid sequence and the KRAS-encoding nucleic acid sequence;
(v) at least 2 iterations of the CTA-encoding nucleic acid sequence and at least 2 iterations of the KRAS-encoding nucleic acid sequence; and/or
(vi) at least 2 iterations of the CTA-encoding nucleic acid sequence and at least 4 iterations of the KRAS-encoding nucleic acid sequence;
optionally wherein the CTA-encoding nucleic acid sequence and the KRAS-encoding nucleic acid sequence are encoded in a single cassette, and wherein the single cassette encodes the amino acid sequence
ELGVMGVYDGREHTVYGEPRKELGVMGVYDGREHTVYGEPRKGIDVKEV
DPTSHSYVLVTSGPRALAETSYVKVLEHVVRVNARVRGPRALAETSYVK
VLEYVIKVSARVRFFFFLNMLGVYDGEEHSVFGEPWFQRNTGEMSSNST
ALALVRPSSSGLINSNTDNNLAVYDLSRDSPQGASALPTTISFTCWRQG
IELMEVDPIGHLYIFATCFQRNTGEMSSNSTALALVRPSSSGLINSNTD
NNLAVYDLSRDSPQGASALPTTISFTCWRQGIELMEVDPIGHLYIFATC
GPRALAETSYVKVLEHVVRVNARVRGPRALAETSYVKVLEYVIKVSARV
RFFFFLNMLGVYDGEEHSVFGEPWGIDVKEVDPTSHSYVLVTSGGSGGV
RAEGRGSLLTCGDVEENPGPMAGMTEYKLVVVGAVGVGKSALTIQLIMT
EYKLVVVGAVGVGKSALTIQLIMTEYKLVVVGAVGVGKSALTIQLIMTE
YKLVVVGAVGVGKSALTIQLIMTEYKLVVVGACGVGKSALTIQLIMTEY
KLVVVGACGVGKSALTIQLIMTEYKLVVVGACGVGKSALTIQLIMTEYK
LVVVGACGVGKSALTIQLIQ.

7.-10. (canceled)

11. The vaccine system of claim 1, wherein the CTA-encoding nucleic acid sequence A is represented by E_{A} and the KRAS-encoding nucleic acid sequence B is represented by E_{B}, and the antigen-encoding vaccine system comprises:
(a) a nucleic acid sequence comprising at least two iterations of E_{A}; or
(b) a nucleic acid sequence comprising at least two iterations of E_{B}; or
(c) a nucleic acid sequence comprising at least two iterations of E_{A} and a nucleic acid sequence comprising at least two iterations of E_{B}; or
(d) a nucleic acid sequence comprising at least two iterations of E_{A} and at least two iterations of E_{B}, and
wherein each iteration of E_{A} and/or E_{B}, respectively, comprises identical nucleic acid sequences,
optionally wherein E_{A} and E_{B} are encoded in a single cassette or are encoded on separate vectors, further optionally wherein if E_{A} and E_{B} are encoded on separate vectors the system comprises a mixture of the separate vectors;
optionally wherein the at least two iterations comprises:
(i) a number of iterations sufficient to stimulate a greater immune response relative to an antigen-encoding nucleic acid sequence comprising a single iteration of the at least one epitope-encoding nucleic acid sequence; and/or
(ii) a number of iterations sufficient to stimulate an immune response, and a single iteration of the at least one epitope-encoding nucleic acid sequence is insufficient to stimulate the immune response or insufficient to stimulate a detectable immune response;
optionally wherein immune response is:
a. an expansion of epitope-specific T cells following in vivo immunization with the composition for delivery of the antigen expression system; or
b. increased activation of epitope-specific T cells and/or increased epitope-specific killing by epitope-specific T cells following in vivo immunization with the composition for delivery of the antigen expression system.

12.-13. (canceled)

14. The vaccine system of claim 11, wherein the system comprises;
(a) at least 2, at least 3, at least 4, at least 5, at least 6, at least 7, or at least 8 iterations of one or both of E_{A} and E_{B};
(b) at least 2 iterations of E_{A};
(c) at least 2 or at least 4 iterations of E_{B};
(d) a nucleic acid sequence C (E_{C}), wherein E_{C} encodes one MHC epitope, wherein the MHC epitope encoded by E_{C} is and distinct and non-identical with respect to the MHC epitope encoded by E_{A} and the MHC epitope encoded by E, optionally wherein E_{C} encodes a non-identical CTA-associated MHC class I epitope with respect to the MHC epitope encoded by E_{A} or a non-identical KRAS-associated MHC class I epitope with respect to the MHC epitope encoded by E_{B};
optionally wherein the system comprises at least 2 iterations of E_{A} and at least 2 or at least 4 iterations of E_{B}.

15.-21. (canceled)

22. An antigen-encoding vaccine system, wherein the antigen-encoding vaccine system comprises:
(A) one or more vectors each comprising:
(a) a vector backbone, wherein the backbone comprises:
(i) at least one promoter nucleotide sequence, and
(ii) at least one polyadenylation (poly(A)) sequence; and
(b) a cassette, wherein the cassette comprises at least one antigen-encoding nucleic acid sequence, comprising:
(i) at least 2 CTA-encoding nucleic acid sequences each encoding a distinct, non-identical CTA-associated MHC class I epitope, wherein each of the CTA-encoding nucleic acid sequences optionally comprises a 5′ linker sequence and/or a 3′ linker sequence, optionally wherein at least one of the CTA-encoding nucleic acid sequences comprises two or more iterations, wherein each iteration of the CTA-encoding nucleic acid sequence comprises an identical nucleic acid sequence, or
(ii) at least one CTA-encoding nucleic acid sequence encoding a CTA-associated MHC class I epitope, wherein at least one of the at least one CTA-encoding nucleic acid sequences comprises two or more iterations, wherein each iteration of the CTA-encoding nucleic acid sequence comprises an identical nucleic acid sequence, and wherein the CTA-encoding nucleic acid sequences optionally comprises a 5′ linker sequence and/or a 3′ linker sequence, optionally comprising at least 2 CTA-encoding nucleic acid sequences each encoding a distinct, non-identical CTA-associated MHC class I epitope; or
(B) one or more vectors, wherein each of the one or more vectors independently comprise:
(a) a vector backbone comprising, wherein the backbone comprises:
(i) a promoter nucleotide sequence; and
(ii) a polyadenylation (poly(A)) sequence, and
optionally wherein the vector backbone comprises an adenoviral vector or a self-amplifying viral vector, optionally wherein the adenoviral vector comprises a chimpanzee adenovirus vector, optionally wherein the chimpanzee adenovirus vector is a ChAdV68 vector, or optionally wherein the self-amplifying viral vector comprises an alphavirus vector, optionally wherein the alphavirus vector is a Venezuelan equine encephalitis virus vector; and
(b) a cassette, wherein the cassette comprises:
(i) the CTA-encoding nucleic acid sequence; or
(ii) the KRAS-encoding nucleic acid sequence; or
(iii) both the CTA-encoding nucleic acid sequence and the KRAS-encoding nucleic acid sequence.

23. (canceled)

24. The vaccine system of claim 1, wherein the CTA-encoding nucleic acid sequence comprises one or more distinct CTA-encoding nucleic acid sequences encoding each encoding a CTA-associated MHC class I epitope and at least one or each of the CTA-associated MHC class I epitopes is selected from the group consisting of: a MAGEA1 MHC class I epitope encoding nucleic acid sequence, a MAGEA3 MHC class I epitope encoding nucleic acid sequence, a MAGEA4 MHC class I epitope encoding nucleic acid sequence, a MAGEA6 MHC class I epitope encoding nucleic acid sequence, a MAGEA8 MHC class I epitope encoding nucleic acid sequence, a MAGEA11 MHC class I epitope encoding nucleic acid sequence, a MAGEB2 MHC class I epitope encoding nucleic acid sequence, a CTCFL MHC class I epitope encoding nucleic acid sequence, and a CT83 MHC class I epitope encoding nucleic acid sequence; optionally wherein at least one or each of the CTA-associated MHC class I epitopes is selected from the group consisting of: FVQENYLEY, EVDPTSHSY, NTDNNLAVY, EVDPIGHLY, GVYDGREHTV, ALREEGEGV, KVLEYVIKV, GVYDGEEHSV, KLVELEHTL, AETSYVKVL, KVLEHVVRV, EADPTGHSY, SALPTTISF, GVYDGREHTVY, TQHFVQENY, EYVIKVSAR, LVRPSSSGL, GEMSSNSTAL, TVYGEPRKL, ALAETSYVK, TSYVKVLEH, YPSLREAAL, ALLEEEEGV, GPRQSLQQC, IAYPSLREAAL, and MEVDPIGHL.

25. (canceled)

26. The vaccine system of claim 1, wherein the CTA-encoding nucleic acid sequence;
(A) encodes:
each of a MAGEA1 MHC class I epitope encoding nucleic acid sequence, a MAGEA4 MHC class I epitope encoding nucleic acid sequence, a MAGEB2 MHC class I epitope encoding nucleic acid sequence, a MAGEA11 MHC class I epitope encoding nucleic acid sequence, a MAGEA3 MHC class I epitope encoding nucleic acid sequence, and a CT83 MHC class I epitope encoding nucleic acid sequence,
each of a MAGEA6 MHC class I epitope encoding nucleic acid sequence, a MAGEA11 MHC class I epitope encoding nucleic acid sequence, a MAGEA3 MHC class I epitope encoding nucleic acid sequence, and a CT83 MHC class I epitope encoding nucleic acid sequence,
each of a MAGEA1 MHC class I epitope encoding nucleic acid sequence, a MAGEA4 MHC class I epitope encoding nucleic acid sequence, a MAGEB2 MHC class I epitope encoding nucleic acid sequence, a MAGEA11 MHC class I epitope encoding nucleic acid sequence, and a CT83 MHC class I epitope encoding nucleic acid sequence, or
each of a MAGEA3 MHC class I epitope encoding nucleic acid sequence, a MAGEA4 MHC class I epitope encoding nucleic acid sequence, a MAGEB2 MHC class I epitope encoding nucleic acid sequence, a MAGEA11 MHC class I epitope encoding nucleic acid sequence, and a CT83 MHC class I epitope encoding nucleic acid sequence; and/or
(B) comprises:
(a) each of a CT83 MHC class I epitope encoding nucleic acid sequence, a MAGEA4 MHC class I epitope encoding nucleic acid sequence, a MAGEA1 MHC class I epitope encoding nucleic acid sequence, a MAGEA8, a MAGEB2 MHC class I epitope encoding nucleic acid sequence, a MAGEA3 MHC class I epitope encoding nucleic acid sequence, and a MAGEA11 MHC class I epitope encoding nucleic acid sequence, optionally wherein the CTA-encoding nucleic acid sequence encodes each of the CTA-associated MHC class I epitopes NTDNNLAVY, LVRPSSSGL, GEMSSNSTAL, GVYDGREHTV, GVYDGREHTVY, ALAETSYVK, KVLEHVVRV, SALPTTISF, GVYDGEEHSV, KVLEYVIKV, AETSYVKVL, EYVIKVSAR, EVDPIGHLY, MEVDPIGHL, and EVDPTSHSY, further optionally wherein the CTA-encoding nucleic acid sequence encodes the amino acid sequence ELGVMGVYDGREHTVYGEPRKELGVMGVYDGREHTVYGEPRKGIDVKEV DPTSHSYVLVTSGPRALAETSYVKVLEHVVRVNARVRGPRALAETSYVKVL EYVIKVSARVRFFFFLNMLGVYDGEEHSVFGEPWFORNTGEMSSNSTALAL VRPSSSGLINSNTDNNLAVYDLSRDSPQGASALPTTISFTCWROGIELMEVDPI GHLYIFATCFORNTGEMSSNSTALALVRPSSSGLINSNTDNNLAVYDLSRDSP QGASALPTTISFTCWROGIELMEVDPIGHLYIFATCGPRALAETSYVKVLEHV VRVNARVRGPRALAETSYVKVLEYVIKVSARVRFFFFLNMLGVYDGEEHSV FGEPWGIDVKEVDPTSHSYVLVTS; and/or
(b) each of a CT83 MHC class I epitope encoding nucleic acid sequence, a MAGEA4 MHC class I epitope encoding nucleic acid sequence, a MAGEA1 MHC class I epitope encoding nucleic acid sequence, a MAGEA8, a MAGEA6 MHC class I epitope encoding nucleic acid sequence, a CTCFL MHC class I epitope encoding nucleic acid sequence, and a MAGEA3 MHC class I epitope encoding nucleic acid sequence, optionally wherein the CTA-encoding nucleic acid sequence encodes each of the CTA-associated MHC class I epitopes NTDNNLAVY, LVRPSSSGL, GEMSSNSTAL, KLVELEHTL, GVYDGREHTV, GVYDGREHTVY, ALAETSYVK, KVLEHVVRV, ALLEEEEGV, YPSLREAAL, IAYPSLREAAL, AETSYVKVL, FVQENYLEY, EVDPIGHLY, MEVDPIGHL, and GPRQSLQQC, further optionally wherein the CTA-encoding nucleic acid sequence encodes the amino acid sequence
GPRALAETSYVKVLEHVVRVNARVRIAYPSLREAALLEEEEGVWLEEGP
RQSLQQCVAISLLTQYFVQENYLEYRQVPGMVENKLVELEHTLLSKGIE
LMEVDPIGHLYIFATCQRNTGEMSSNSTALALVRPSSSGLINSNTDNNL
AVYDLSRWEELGVMGVYDGREHTVYGEPRKLLTQDQRNTGEMSSNSTAL
ALVRPSSSGLINSNTDNNLAVYDLSRWEELGVMGVYDGREHTVYGEPRK
LLTQDWMVENKLVELEHTLLSKGIELMEVDPIGHLYIFATCPRALAETS
YVKVLEHVVRVNARVRIAYPSLREAALLEEEEGVWLEEGPRQSLQQCVA
ISLLTQYFVQENYLEYRQVPG.

27.-32. (canceled)

33. The vaccine system of claim 1, wherein the KRAS-encoding nucleic acid sequence comprises one or more distinct KRAS-encoding nucleic acid sequences each encoding a KRAS-associated MHC class I epitope, wherein:
(a) at least one or each of the KRAS-associated MHC class I epitopes comprises a KRAS neoepitope independently comprising:
(i) a KRAS G12C mutation, a KRAS G12V mutation, a KRAS G12D mutation, or a KRAS Q61H mutation, or
(ii) a KRAS G12C mutation or a KRAS G12V mutation; and/or
(b) the KRAS-encoding nucleic acid sequence encodes each of a KRAS neoepitope comprising:
(i) a G12C mutation and a KRAS neoepitope comprising a KRAS G12V mutation, or
(ii) a KRAS neoepitope comprising a KRAS G12C mutation, a KRAS neoepitope comprising a KRAS G12V mutation, a KRAS neoepitope comprising a KRAS G12D mutation, and a KRAS neoepitope comprising a KRAS Q61H mutation;
optionally wherein the KRAS neoepitope comprising:
(a) the KRAS G12C mutation comprises:
(i) the amino acid sequence KLVVVGACGV, VVVGACGVGK, or GACGVGKSAL, and combinations thereof, or
(ii) the amino acid sequence VVVGACGVGK or KLVVVGACGV, and combinations thereof;
(b) the KRAS neoepitope comprising the KRAS G12V mutation comprises:
(i) the amino acid sequence KLVVVGAVGV, VVVGAVGVGK, AVGVGKSAL, or GAVGVGKSAL, and combinations thereof, or
(ii) the amino acid sequence VVGAVGVGK, VVVGAVGVGK, or AVGVGKSAL, and combinations thereof;
(c) the KRAS neoepitope comprising the KRAS G12D mutation comprises the amino acid sequence VVGADGVGK or VVVGADGVGK; and/or
(d) the KRAS neoepitope comprising the KRAS Q61H mutation comprises the amino acid sequence ILDTAGHEEY:
further optionally wherein the KRAS-encoding nucleic acid sequence encodes each of the amino acid sequences VVVGACGVGK, VVVGADGVGK, VVGAVGVGK, and ILDTAGHEEY: optionally wherein:
(a) the KRAS-encoding nucleic acid sequence encodes the amino acid sequence MTEYKLVVVGAVGVGKSALTIQLIQMTEYKLVVVGAVGVGKSALTIQLIQM TEYKLVVVGAVGVGKSALTIQLIQMTEYKLVVVGAVGVGKSALTIQLIQMT EYKLVVVGACGVGKSALTIQLIQMTEYKLVVVGACGVGKSALTIQLIQMTE YKLVVVGACGVGKSALTIQLIQMTEYKLVVVGACGVGKSALTIQLIQ; or
(b) the KRAS-encoding nucleic acid sequence encodes the amino acid sequence
MTEYKLVVVGACGVGKSALTIQLIQMTEYKLVVVGADGVGKSALTIQLI
QETCLLDILDTAGHEEYSAMRDQYMRMTEYKLVVVGADGVGKSALTIQL
IQMTEYKLVVVGAVGVGKSALTIQLIQMTEYKLVVVGACGVGKSALTIQ
LIQETCLLDILDTAGHEEYSAMRDQYMRMTEYKLVVVGADGVGKSALTI
QLIQMTEYKLVVVGAVGVGKSALTIQLIQMTEYKLVVVGACGVGKSALT
IQLIQETCLLDILDTAGHEEYSAMRDQYMRMTEYKLVVVGADGVGKSAL
TIQLIQMTEYKLVVVGAVGVGKSALTIQLIQETCLLDILDTAGHEEYSA
MRDQYMRMTEYKLVVVGAVGVGKSALTIQLIQMTEYKLVVVGACGVGKS
ALTIQLIQ.

34.-54. (canceled)

55. The vaccine system of claim 1, wherein the CTA-encoding nucleic acid sequence and the KRAS-encoding nucleic acid sequence are encoded in a single cassette, and optionally wherein:
(a) separate promoter nucleotide sequences provide for transcription of one or more of the separate open reading frames encoding the CTA-associated MHC class I epitope and the KRAS-associated MHC class I epitope, respectively, optionally wherein:
(i) the separate promoter nucleotide sequences are different, optionally wherein the separate promoter nucleotide sequences are selected from the group consisting of a CMV, SV40, EF-1, RSV, PGK, HSA, MCK and a EBV promoter sequence, further optionally wherein the promoters comprise a TETr controlled promoter, further optionally wherein the TETr controlled promoter comprises a TETr controlled CMV-derived promoter or a TETr controlled EF-1-derived promoter; or
(ii) each of the separate promoters comprises a subgenomic promoter sequence, optionally wherein the subgenomic promoter sequence comprises a 26S subgenomic promoter sequence;
(b) the CTA-associated MHC class I epitope and the KRAS-associated MHC class I epitope are capable of being expressed as a single polypeptide, and/or
(c) the CTA-associated MHC class I epitope and the KRAS-associated MHC class I epitope are capable of being expressed as a single polypeptide.

56.-62. (canceled)

63. The vaccine system of claim 1, wherein the CTA-encoding nucleic acid sequence and/or the KRAS-encoding nucleic acid sequence, and optionally each CTA-encoding nucleic acid sequence and KRAS-encoding nucleic acid sequence, are described, from 5′ to 3′, by the formula (L5_{b}-N_{c}-L3_{d}),
wherein N comprises a distinct epitope-encoding nucleic acid sequence that encodes the MHC epitope associated with each of the CTA-encoding nucleic acid sequences and the KRAS-encoding nucleic acid sequences, where c=1,
L5 comprises a 5′ linker sequence, where b=0 or 1, and
L3 comprises a 3′ linker sequence, where d=0 or 1;
optionally wherein:
each N encodes an epitope 7-15 amino acids in length, L5 is a native 5′ linker sequence that encodes a native N-terminal amino acid sequence of the epitope, and wherein the 5′ linker sequence encodes a peptide that is at least 2 amino acids in length, and optionally between 2-20 amino acids in length, and
L3 is a native 3′ linker sequence that encodes a native C-terminal amino acid sequence of the epitope, and wherein the 3′ linker sequence encodes a peptide that is at least 2 amino acids in length, and optionally between 2-20 amino acids in length, and
optionally wherein the CTA-encoding nucleic acid sequence and/or the KRAS-encoding nucleic acid sequence encodes a polypeptide that is between 12 and 35 amino acids in length each N encodes an epitope 7-15 amino acids in length,
L5 is a native 5′ linker sequence that encodes a native N-terminal amino acid sequence of the epitope, and wherein the 5′ linker sequence encodes a peptide that is at least 2 amino acids in length, and optionally between 2-20 amino acids in length, and
L3 is a native 3′ linker sequence that encodes a native C-terminal amino acid sequence of the epitope, and wherein the 3′ linker sequence encodes a peptide that is at least 2 amino acids in length, and optionally between 2-20 amino acids in length, and
optionally wherein the CTA-encoding nucleic acid sequence and/or the KRAS-encoding nucleic acid sequence encodes a polypeptide that is between 12 and 35 amino acids in length.

64. (canceled)

65. The vaccine system of claim 1, wherein one or both of the CTA-encoding nucleic acid sequence and the KRAS-encoding nucleic acid sequence, and optionally each of the CTA-encoding nucleic acid sequence and the KRAS-encoding nucleic acid sequences:
(a) encodes an epitope at least 7 amino acids in length;
(b) encodes an epitope 7-15 amino acids in length;
(c) is a nucleotide sequence at least 21 nucleotides in length; and/or
(d) is a nucleotide sequence 75 nucleotides in length.

66.-73. (canceled)

74. The vaccine system of claim 1, wherein:
(a) the composition further comprises a nanoparticulate delivery vehicle, wherein the nanoparticulate delivery vehicle encapsulates the CTA-encoding nucleic acid sequence and/or the KRAS-encoding nucleic acid sequence, optionally wherein the CTA-encoding nucleic acid sequence and the KRAS-encoding nucleic acid sequence are formulated:
(i) in the same nanoparticulate delivery vehicle, or
(ii) in separate nanoparticulate delivery vehicles, and wherein the composition comprises a mixture of the separate nanoparticulate delivery vehicles;
(b) the vaccine system does not encode a non-therapeutic MHC class I or class II epitope nucleic acid sequence comprising a translated, wild-type nucleic acid sequence, wherein the non-therapeutic epitope is predicted to be displayed on an MHC allele of the subject, optionally wherein the non-therapeutic predicted MHC class I or class II epitope sequence is a junctional epitope sequence formed by adjacent sequences in the cassette;
(c) the cassette is integrated between the at least one promoter nucleotide sequence and the at least one poly(A) sequence;
(d) the one or more vectors comprise:
(i) one or more +-stranded RNA vectors,
(ii) a 5′ 7-methylguanosine (m7 g) cap,
(iii) RNA vectors produced by in vitro transcription, and/or
(iv) vectors that are self-replicating within a mammalian cell;
(e) the backbone comprises at least one nucleotide sequence of a Venezuelan equine encephalitis virus, optionally wherein:
(i) the vector backbone comprises at least sequences for nonstructural protein-mediated amplification, a 26S promoter sequence, and a poly(A) sequence encoded by the nucleotide sequence of the Venezuelan equine encephalitis virus, wherein sequences for nonstructural protein-mediated amplification are selected from the group consisting of: an alphavirus 5′ UTR, a 51-nt CSE, a 24-nt CSE, a 26S subgenomic promoter sequence, a 19-nt CSE, an alphavirus 3′ UTR, or combinations thereof, and/or wherein the backbone does not encode structural virion proteins capsid, E2 and E1, and/or
(ii) the Venezuelan equine encephalitis virus comprises the sequence of SEQ ID NO:3 or SEQ ID NO:5 further comprising a deletion between base pair 7544 and 11175, wherein the antigen cassette is inserted at position 7544 to replace the deletion between base pairs 7544 and 11175 as set forth in the sequence of SEQ ID NO:3 or SEQ ID NO:5;
(f) the backbone comprises at least one nucleotide sequence of a chimpanzee adenovirus vector, optionally wherein the chimpanzee adenovirus vector is a ChAdV68 vector, optionally wherein the ChAdV68 vector comprises a ChAdV68 vector backbone comprising:
(i) the sequence set forth in SEQ ID NO:1,
(ii) the sequence set forth in SEQ ID NO:1, except that the sequence is fully deleted or functionally deleted in at least one gene selected from the group consisting of the chimpanzee adenovirus E1A, E1B, E2A, E2B, E3, E4, L1, L2, L3, L4, and L5 genes of the sequence set forth in SEQ ID NO:1, optionally wherein the sequence is fully deleted or functionally deleted in: (1) E1A and E1B: (2) E1A, E1B, and E3; or (3) E1A, E1B, E3, and E4 of the sequence set forth in SEQ ID NO:1,
(iii) a gene or regulatory sequence obtained from the sequence of SEQ ID NO:1, optionally wherein the gene is selected from the group consisting of the chimpanzee adenovirus inverted terminal repeat (ITR), E1A, E1B, E2A, E2B, E3, E4, L1, L2, L3, L4, and L5 genes of the sequence set forth in SEQ ID NO:1,
(iv) a partially deleted E4 gene comprising a deleted or partially-deleted E4orf2 region and a deleted or partially-deleted E4orf3 region, and optionally a deleted or partially-deleted E4orf4 region,
(v) at least nucleotides 2 to 36,518 of the sequence set forth in SEQ ID NO:1 and further comprising: (1) an E1 deletion of at least nucleotides 577 to 3403 of the sequence shown in SEQ ID NO:1, (2) an E3 deletion of at least nucleotides 27,125 to 31,825 of the sequence shown in SEQ ID NO:1, and (3) an E4 deletion of at least nucleotides 34,916 to 35,642 of the sequence shown in SEQ ID NO:1; optionally wherein the antigen cassette is inserted within the E1 deletion
(vi) one or more deletions between base pair number 577 and 3403 or between base pair 456 and 3014, and optionally wherein the vector further comprises one or more deletions between base pair 27,125 and 31,825 or between base pair 27,816 and 31,333 of the sequence set forth in SEQ ID NO:1; or
(vii) one or more deletions between base pair number 3957 and 10346, base pair number 21787 and 23370, and base pair number 33486 and 36193 of the sequence set forth in SEQ ID NO:1, and
optionally wherein the cassette is inserted in the ChAdV vector backbone at the E1 region, E3 region, and/or any deleted AdV region that allows incorporation of the cassette;
(g) the at least one promoter nucleotide sequence is the native subgenomic promoter nucleotide sequence encoded by the backbone, optionally a 26S promoter nucleotide sequence;
(h) the vector comprises multiple subgenomic promoter nucleotide sequence, wherein each subgenomic promoter nucleotide sequence are operably linked to and provide for transcription of one or more separate open reading frames in the cassette;
(i) the at least one promoter sequence is a regulatable promoter, optionally wherein the regulatable promoter is a tetracycline (TET) repressor protein (TETr) controlled promoter, optionally wherein the regulatable promoter comprises multiple TET operator (TETo) sequences 5′ or 3′ of a RNA polymerase binding sequence of the promoter;
(j) each of the MHC class I epitopes is predicted or validated to be capable of presentation by at least one HLA allele, wherein each antigen/HLA pair has an antigen/HLA prevalence of at least 0.01% or 0.1% in a population, optionally wherein the at least one HLA allele is HLA A*01:01, HLA A*02:01, B*44:02, B*44:05, B*40:01, B*40:02, B*41:02, B*35:01, B*15:01, A*33:03, A*02:05, A*11:01, C*03:04, A*29:02, C*15:02, and/or B*07:02; and/or
(k) each of the CTA-associated MHC class I epitope is validated to be capable of presentation by at least one HLA allele, wherein each antigen/HLA pair has an antigen/HLA prevalence of at least 0.1% or 0.5% in a population, optionally wherein the at least one HLA allele is HLA A*01:01, HLA A*02:01, B*44:02, B*44:05, B*40:01, B*40:02, B*41:02, B*35:01, B*15:01, A*33:03, A*02:05, A*11:01, C*03:04, A*29:02, C*15:02, and/or B*07:02.

75.-95. (canceled)

96. A pharmaceutical composition or compositions comprising the vaccine system of claim 1 and a pharmaceutically acceptable carrier, optionally wherein:
(a) the CTA-encoding nucleic acid sequence and the RAS-encoding nucleic acid sequence are co-formulated; or
(b) the CTA-encoding nucleic acid sequence and the KRAS-encoding nucleic acid sequence are formulated in separate pharmaceutical compositions.

97.-98. (canceled)

99. An isolated nucleotide sequence or set of isolated nucleotide sequences comprising the antigen-encoding vaccine system of claim 1, and optionally one or more elements obtained from the sequence of SEQ ID NO:3 or SEQ ID NO:5, optionally wherein the one or more elements are selected from the group consisting of the sequences necessary for nonstructural protein-mediated amplification, the 26S promoter nucleotide sequence, the poly(A) sequence, and the nsP1-4 genes of the sequence set forth in SEQ ID NO:3 or SEQ ID NO:5, and optionally wherein the nucleotide sequence is cDNA.

100. A vector or set of vectors comprising the nucleotide sequence of claim 99.

101. An isolated cell comprising the nucleotide sequence or set of isolated nucleotide sequences of claim 99, optionally wherein the cell is a BHK-21, CHO, HEK293 or variants thereof, 911, HeLa, A549, LP-293, PER.C6, or AE1-2a cell.

102. A kit comprising the vaccine system of claim 1 and instructions for use, optionally wherein:
(a) the kit comprises the CTA-encoding nucleic acid sequence and the KRAS-encoding nucleic acid sequence co-formulated in a pharmaceutical composition; or
(b) the kit comprises a first pharmaceutical composition comprising the CTA-encoding nucleic acid sequence and a second pharmaceutical composition comprising the KRAS-encoding nucleic acid sequence.

103.-104. (canceled)

105. A method for treating a subject with cancer, optionally wherein the cancer is non-small cell lung cancer (NSCLC), or a method for stimulating an immune response in a subject, the method comprising administering to the subject the vaccine system of claim 1, optionally wherein:
(a) the subject expresses at least one HLA allele predicted or known to present the CTA-associated MHC class I epitope, optionally wherein the at least one HLA allele is HLA A*01:01, HLA A*02:01, B*44:02, B*44:05, B*40:01, B*40:02, B*41:02, B*35:01, B*15:01, A*33:03, A*02:05, A*11:01, C*03:04, A*29:02, C*15:02, and/or B*07:02;
(b) the method further comprises administering to the subject a second vaccine composition, optionally wherein:
(i) the second vaccine composition is administered prior to or subsequent to the administration of the vaccine system, or the second vaccine composition is administered subsequent to the administration of the vaccine system,
(ii) the second vaccine composition is the same as or is different from the vaccine system.

106.-113. (canceled)

114. A method of manufacturing comprising:
(A) manufacturing the one or more vectors of the vaccine system of claim 1, the method comprising:
(a) obtaining a linearized DNA sequence comprising the backbone and the cassette;
(b) in vitro transcribing the linearized DNA sequence by addition of the linearized DNA sequence to an in vitro transcription reaction containing all the necessary components to transcribe the linearized DNA sequence into RNA, optionally further comprising in vitro addition of the m7 g cap to the resulting RNA; and
(c) isolating the one or more vectors from the in vitro transcription reaction; and/or
(B) manufacturing the vaccine system of claim 1 for delivery of the antigen-encoding vaccine system, the method comprising:
(a) providing components for the nanoparticulate delivery vehicle;
(b) providing the antigen-encoding vaccine system; and
(c) contacting the components for the nanoparticulate delivery vehicle and the antigen expression system under conditions sufficient for the nanoparticulate delivery vehicle and the antigen-encoding vaccine system to produce a delivery composition for delivery of the antigen-encoding vaccine system,
optionally wherein:
(i) the conditions are provided by microfluidic mixing,
(ii) the CTA-encoding nucleic acid sequence and the KRAS-encoding nucleic acid sequence are encoded:
a. in the same cassette and/or vector,
b. on separate vectors and mixed prior to the contacting step (c),
c. on separate vectors, and wherein the separate vectors are independently contacted with the components for the nanoparticulate delivery vehicle to produce a first delivery composition comprising the CTA-encoding nucleic acid sequence and a second delivery composition comprising the KRAS-encoding nucleic acid sequence, optionally wherein the first delivery composition and the second delivery composition are mixed subsequent to the contacting step (c).

115.-120. (canceled)

121. A method for treating a subject with a disease, optionally wherein the disease is cancer, the method comprising administering to the subject an antigen-encoding vaccine system, wherein the antigen-encoding vaccine system comprises:
(A) (a) a Cancer Testis Antigen (CTA)-encoding nucleic acid sequence encoding a CTA-associated MHC class I epitope; and
(b) a KRAS-encoding nucleic acid sequence encoding a KRAS-associated MHC class I epitope; or
(B) (a) a CTA-encoding nucleic acid sequence A (E_{A}); and
(b) a KRAS-encoding nucleic acid sequence B (E_{B}),
wherein E_{A} and E_{B} each encode one MHC epitope,
wherein E_{A} encodes a CTA-associated MHC class I epitope, and
wherein E_{B} encodes a KRAS-associated MHC class I epitope;
optionally wherein the antigen-encoding vaccine system is administered as:
(a) a priming dose, or
(b) as one or more boosting doses, optionally wherein the boosting dose is:
(i) different than the priming dose, optionally wherein:
a. the priming dose comprises a chimpanzee adenovirus vector and the boosting dose comprises an alphavirus vector, or
b. the priming dose comprises an alphavirus vector and the boosting dose comprises a chimpanzee adenovirus vector; or
(ii) the same as the priming dose;
optionally wherein the injection site of the one or more boosting doses is as close as possible to the injection site of the priming dose;
further optionally wherein:
(a) the method comprises determining or having determined the HLA-haplotype of the subject, optionally wherein the HLA-haplotype determined of the subject comprises an HLA allele predicted or validated to present at least one of the CTA-associated MHC class I epitopes encoded by the antigen-encoding cassette, optionally wherein the HLA allele is HLA A*01:01, HLA A*02:01, B*44:02, B*44:05, B*40:01, B*40:02, B*41:02, B*35:01, B*15:01, A*33:03, A*02:05, A*11:01, C*03:04, A*29:02, C*15:02, and/or B*07:02; and/or
(b) the CTA-encoding nucleic acid sequence and the KRAS-encoding nucleic acid sequence are co-administered, optionally wherein the CTA-encoding nucleic acid sequence and the KRAS-encoding nucleic acid sequence are:
(i) co-formulated in a single delivery composition;
(ii) formulated in a separate delivery compositions, optionally wherein the separate delivery compositions are administered at separate injection sites and/or are mixed prior to co-administration, optionally wherein the administration at separate injection sites comprises bilateral administration.

122.-137. (canceled)


SEQUENCE LISTING (75 sequences)

SEQ ID NO: 1 - DNA, 36519 bp, synthetic construct
  misc_feature 1..36519: note = Description of Artificial Sequence: Synthetic polynucleotide
  source 1..36519: mol_type = other DNA
ccatcttcaataatatacctcaaactttttgtgcgcgttaatatgcaaatgaggcgtttgaatttggggaggaagggcggtgattggtcgagggatgagcgaccgttaggggcggggcgagtgacgttttgatgacgtggttgcgaggaggagccagtttgcaagttctcgtgggaaaagtgacgtcaaacgaggtgtggtttgaacacggaaatactcaattttcccgcgctctctgacaggaaatgaggtgtttctgggcggatgcaagtgaaaacgggccattttcgcgcgaaaactgaatgaggaagtgaaaatctgagtaatttcgcgtttatggcagggaggagtatttgccgagggccgagtagactttgaccgattacgtgggggtttcgattaccgtgtttttcacctaaatttccgcgtacggtgtcaaagtccggtgtttttacgtaggtgtcagctgatcgccagggtatttaaacctgcgctctccagtcaagaggccactcttgagtgccagcgagaagagttttctcctccgcgccgcgagtcagatctacactttgaaagatgaggcacctgagagacctgcccgatgagaaaatcatcatcgcttccgggaacgagattctggaactggtggtaaatgccatgatgggcgacgaccctccggagccccccaccccatttgagacaccttcgctgcacgatttgtatgatctggaggtggatgtgcccgaggacgatcccaatgaggaggcggtaaatgatttttttagcgatgccgcgctgctagctgccgaggaggcttcgagctctagctcagacagcgactcttcactgcatacccctagacccggcagaggtgagaaaaagatccccgagcttaaaggggaagagatggacttgcgctgctatgaggaatgcttgcccccgagcgatgatgaggacgagcaggcgatccagaacgcagcgagccagggagtgcaagccgccagcgagagctttgcgctggactgcccgcctctgcccggacacggctgtaagtcttgtgaatttcatcgcatgaatactggagataaagctgtgttgtgtgcactttgctatatgagagcttacaaccattgtgtttacagtaagtgtgattaagttgaactttagagggaggcagagagcagggtgactgggcgatgactggtttatttatgtatatatgttctttatataggtcccgtctctgacgcagatgatgagacccccactacaaagtccacttcgtcacccccagaaattggcacatctccacctgagaatattgttagaccagttcctgttagagccactgggaggagagcagctgtggaatgtttggatgacttgctacagggtggggttgaacctttggacttgtgtacccggaaacgccccaggcactaagtgccacacatgtgtgtttacttgaggtgatgtcagtatttatagggtgtggagtgcaataaaaaatgtgttgactttaagtgcgtggtttatgactcaggggtggggactgtgagtatataagcaggtgcagacctgtgtggttagctcagagcggcatggagatttggacggtcttggaagactttcacaagactagacagctgctagagaacgcctcgaacggagtctcttacctgtggagattctgcttcggtggcgacctagctaggctagtctacagggccaaacaggattatagtgaacaatttgaggttattttgagagagtgttctggtctttttgacgctcttaacttgggccatcagtctcactttaaccagaggatttcgagagcccttgattttactactcctggcagaaccactgcagcagtagccttttttgcttttattcttgacaaatggagtcaagaaacccatttcagcagggattaccagctggatttcttagcagtagctttgtggagaacatggaagtgccagcgcctgaatgcaatctccggctacttgccggtacagccgctagacactctgaggatcctgaatctccaggagagtcccagggcacgccaacgtcgccagcagcagcagcaggaggaggatcaagaagagaacccgagagccggcctggaccctccggcggaggaggaggagtagctgacctgtttcctgaactgcgccgggtgctgactaggtcttcgagtggtcgggagagggggattaagcgggagaggcatgatgagactaatcacagaactgaactgactgtgggtctgatgagtcgcaagcgcccagaaacagtgtggtggcatgaggtgcagtcgactggcacagatgaggtgtcggtgatgcatgagaggttttctctagaacaagtcaagacttgttggttagagcctgaggatgattgggaggtagccatcaggaattatgccaagctggctctgaggccagacaagaagtacaagattactaagctgataaatatcagaaatgcctgctacatctcagggaatggggctgaagtggagatctgtctccaggaaagggtggctttcagatgctgcatgatgaatatgtacccgggagtggtgggcatggatggggttacctttatgaacatgaggttcaggggagatgggtataatggcacggtctttatggccaataccaagctgacagtccatggctgctccttctttgggtttaataacacctgcatcgaggcctggggtcaggtcggtgtgaggggctgcagtttttcagccaactggatgggggtcgtgggcaggaccaagagtatgctgtccgtgaagaaatgcttgtttgagaggtgccacctgggggtgatgagcgagggcgaagccagaatccgccactgcgcctctaccgagacgggctgctttgtgctgtgcaagggcaatgctaagatcaagcataatatgatctgtggagcctcggacgagcgcggctaccagatgctgacctgcgccggcgggaacagccatatgctggccaccgtacatgtggcttcccatgctcgcaagccctggcccgagttcgagcacaatgtcatgaccaggtgcaatatgcatctggggtcccgccgaggcatgttcatgccctaccagtgcaacctgaattatgtgaaggtgctgctggagcccgatgccatgtccagagtgagcctgacgggggtgtttgacatgaatgtggaggtgtggaagattctgagatatgatgaatccaagaccaggtgccgagcctgcgagtgcggagggaagcatgccaggttccagcccgtgtgtgtggatgtgacggaggacctgcgacccgatcatttggtgttgccctgcaccgggacggagttcggttccagcggggaagaatctgactagagtgagtagtgttctggggcgggggaggacctgcatgagggccagaataactgaaatctgtgcttttctgtgtgttgcagcagcatgagcggaagcggctcctttgagggaggggtattcagcccttatctgacggggcgtctcccctcctgggcgggagtgcgtcagaatgtgatgggatccacggtggacggccggcccgtgcagcccgcgaactcttcaaccctgacctatgcaaccctgagctcttcgtcgttggacgcagctgccgccgcagctgctgcatctgccgccagcgccgtgcgcggaatggccatgggcgccggctactacggcactctggtggccaactcgagttccaccaataatcccgccagcctgaacgaggagaagctgttgctgctgatggcccagctcgaggccttgacccagcgcctgggcgagctgacccagcaggtggctcagctgcaggagcagacgcgggccgcggttgccacggtgaaatccaaataaaaaatgaatcaataaataaacggagacggttgttgattttaacacagagtctgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcatggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcctttgtgcccgcccaggttttccatgcactcatccatgatgatggcgatgggcccgtgggcggcggcctgggcaaagacgtttcgggggtcggacacatcatagttgtggtcctgggtgaggtcatcataggccattttaatgaatttggggcggagggtgccggactgggggacaaaggtaccctcgatcccgggggcgtagttcccctcacagatctgcatctcccaggctttgagctcggagggggggatcatgtccacctgcggggcgataaagaacacggtttccggggcgggggagatgagctgggccgaaagcaagttccggagcagctgggacttgccgcagccggtggggccgtagatgaccccgatgaccggctgcaggtggtagttgagggagagacagctgccgtcctcccggaggaggggggccacctcgttcatcatctcgcgcacgtgcatgttctcgcgcaccagttccgccaggaggcgctctccccccagggataggagctcctggagcgaggcgaagtttttcagcggcttgagtccgtcggccatgggcattttggagagggtttgttgcaagagttccaggcggtcccagagctcggtgatgtgctctacggcatctcgatccagcagacctcctcgtttcgcgggttgggacggctgcgggagtagggcaccagacgatgggcgtccagcgcagccagggtccggtccttccagggtcgcagcgtccgcgtcagggtggtctccgtcacggtgaaggggtgcgcgccgggctgggcgcttgcgagggtgcgcttcaggctcatccggctggtcgaaaaccgctcccgatcggcgccctgcgcgtcggccaggtagcaattgaccatgagttcgtagttgagcgcctcggccgcgtggcctttggcgcggagcttacctttggaagtctgcccgcaggcgggacagaggagggacttgagggcgtagagcttgggggcgaggaagacggactcgggggcgtaggcgtccgcgccgcagtgggcgcagacggtctcgcactccacgagccaggtgaggtcgggctggtcggggtcaaaaaccagtttcccgccgttctttttgatgcgtttcttacctttggtctccatgagctcgtgtccccgctgggtgacaaagaggctgtccgtgtccccgtagaccgactttatgggccggtcctcgagcggtgtgccgcggtcctcctcgtagaggaaccccgcccactccgagacgaaagcccgggtccaggccagcacgaaggaggccacgtgggacgggtagcggtcgttgtccaccagcgggtccaccttttccagggtatgcaaacacatgtccccctcgtccacatccaggaaggtgattggcttgtaagtgtaggccacgtgaccgggggtcccggccgggggggtataaaagggtgcgggtccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccggcggagatgcctttcaagagcccctcgtccatctggtcagaaaagacgatctttttgttgtcgagcttggtggcgaaggagccgtagagggcgttggagaggagcttggcgatggagcgcatggtctggtttttttccttgtcggcgcgctccttggcggcgatgttgagctgcacgtactcgcgcgccacgcacttccattcggggaagacggtggtcagctcgtcgggcacgattctgacctgccagccccgattatgcagggtgatgaggtccacactggtggccacctcgccgcgcaggggctcattagtccagcagaggcgtccgcccttgcgcgagcagaaggggggcagggggtccagcatgacctcgtcgggggggtcggcatcgatggtgaagatgccgggcaggaggtcggggtcaaagtagctgatggaagtggccagatcgtccagggcagcttgccattcgcgcacggccagcgcgcgctcgtagggactgaggggcgtgccccagggcatgggatgggtaagcgcggaggcgtacatgccgcagatgtcgtagacgtagaggggctcctcgaggatgccgatgtaggtggggtagcagcgccccccgcggatgctggcgcgcacgtagtcatacagctcgtgcgagggggcgaggagccccgggcccaggttggtgcgactgggcttttcggcgcggtagacgatctggcggaaaatggcatgcgagttggaggagatggtgggcctttggaagatgttgaagtgggcgtggggcagtccgaccgagtcgcggatgaagtgggcgtaggagtcttgcagcttggcgacgagctcggcggtgactaggacgtccagagcgcagtagtcgagggtctcctggatgatgtcatacttgagctgtcccttttgtttccacagctcgcggttgagaaggaactcttcgcggtccttccagtactcttcgagggggaacccgtcctgatctgcacggtaagagcctagcatgtagaactggttgacggccttgtaggcgcagcagcccttctccacggggagggcgtaggcctgggcggccttgcgcagggaggtgtgcgtgagggcgaaagtgtccctgaccatgaccttgaggaactggtgcttgaagtcgatatcgtcgcagcccccctgctcccagagctggaagtccgtgcgcttcttgtaggcggggttgggcaaagcgaaagtaacatcgttgaagaggatcttgcccgcgcggggcataaagttgcgagtgatgcggaaaggttggggcacctcggcccggttgttgatgacctgggcggcgagcacgatctcgtcgaagccgttgatgttgtggcccacgatgtagagttccacgaatcgcggacggcccttgacgtggggcagtttcttgagctcctcgtaggtgagctcgtcggggtcgctgagcccgtgctgctcgagcgcccagtcggcgagatgggggttggcgcggaggaaggaagtccagagatccacggccagggcggtttgcagacggtcccggtactgacggaactgctgcccgacggccattttttcgggggtgacgcagtagaaggtgcgggggtccccgtgccagcgatcccatttgagctggagggcgagatcgagggcgagctcgacgagccggtcgtccccggagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccattggtggcgacgtcgatggcttgcagggtcccgtgcccctggggtgtgaccaccgtcccccgtttcttcttgggcggctggggcgacgggggcggtgcctcttccatggttagaagcggcggcgaggacgcgcgccgggcggcaggggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcttgaaggtctccgcggccggcgcgctccacggtggccgcgaggtcgttggagatgcggcccatgagctgcgagaaggcgttcatgcccgcctcgttccagacgcggctgtagaccacgacgccctcgggatcgcgggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaaacgttccatggcctcgtaaaagtccacggcgaagttgaaaaactgggagttgcgcgccgagacggtcaactcctcctccagaagacggatgagctcggcgatggtggcgcgcacctcgcgctcgaaggcccccgggagttcctccacttcctcttcttcctcctccactaacatctcttctacttcctcctcaggcggcagtggtggcgggggagggggcctgcgtcgccggcggcgcacgggcagacggtcgatgaagcgctcgatggtctcgccgcgccggcgtcgcatggtctcggtgacggcgcgcccgtcctcgcggggccgcagcgtgaagacgccgccgcgcatctccaggtggccgggggggtccccgttgggcagggagagggcgctgacgatgcatcttatcaattgccccgtagggactccgcgcaaggacctgagcgtctcgagatccacgggatctgaaaaccgctgaacgaaggcttcgagccagtcgcagtcgcaaggtaggctgagcacggtttcttctggcgggtcatgttggttgggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccaggtccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcttgctggatctgggtgagggtggtctggaagtcatcaaagtcgacgaagcggtggtaggctccggtgttgatggtgtaggagcagttggccatgacggaccagttgacggtctggtggcccggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtattggcactcccgtctcgacccaagcctgcaccaaccctccaggatacggaggcgggtcgttttgcaacttttttttggaggccggatgagactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagaccccatagccagccgacttctccagttacggagcgagcccctcttttgttttgtttgtttttgccagatgcatcccgtactgcggcagatgcgcccccaccaccctccaccgcaacaacagccccctccacagccggcgcttctgcccccgccccagcagcaacttccagccacgaccgccgcggccgccgtgagcggggctggacagagttatgatcaccagctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaagcgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagtttgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgtgcggtgagcgccagcaggcggcgcgagctgagcgaccaggagctgatgcatagtctgcagcgggccctgaccggggccgggaccgagggggagagctactttgacatgggcgcggacctgcactggcagcccagccgccgggccttggaggcggcggcaggaccctacgtagaagaggtggacgatgaggtggacgaggagggcgagtacctggaagactgatggcgcgaccgtatttttgctagatgcaacaacaacagccacctcctgatcccgcgatgcgggcggcgctgcagagccagccgtccggcattaactcctcggacgattggacccaggccatgcaacgcatcatggcgctgacgacccgcaaccccgaagcctttagacagcagccccaggccaaccggctctcggccatcctggaggccgtggtgccctcgcgctccaaccccacgcacgagaaggtcctggccatcgtgaacgcgctggtggagaacaaggccatccgcggcgacgaggccggcctggtgtacaacgcgctgctggagcgcgtggcccgctacaacagcaccaacgtgcagaccaacctggaccgcatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggctttcaagaacttgcagggcctgtggggcgtgcaggccccggtcggggaccgcgcgacggtgtcgagcctgctgacgccgaactcgcgcctgctgctgctgctggtggcccccttcacggacagcggcagcatcaaccgcaactcgtacctgggctacctgattaacctgtaccgcgaggccatcggccaggcgcacgtggacgagcagacctaccaggagatcacccacgtgagccgcgccctgggccaggacgacccgggcaacctggaagccaccctgaactttttgctgaccaaccggtcgcagaagatcccgccccagtacgcgctcagcaccgaggaggagcgcatcctgcgttacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctgaatccccactggctcccgccgccggggttctacacgggcgagtacgacatgcccgaccccaatgacgggttcctgtgggacgatgtggacagcagcgtgttctccccccgaccgggtgctaacgagcgccccttgtggaagaaggaaggcagcgaccgacgcccgtcctcggcgctgtccggccgcgagggtgctgccgcggcggtgcccgaggccgccagtcctttcccgagcttgcccttctcgctgaacagtatccgcagcagcgagctgggcaggatcacgcgcccgcgcttgctgggcgaagaggagtacttgaatgactcgctgttgagacccgagcgggagaagaacttccccaataacgggatagaaagcctggtggacaagatgagccgctggaagacgtatgcgcaggagcacagggacgatccccgggcgtcgcagggggccacgagccggggcagcgccgcccgtaaacgccggtggcacgacaggcagcggggacagatgtgggacgatgaggactccgccgacgacagcagcgtgttggacttgggtgggagtggtaacccgttcgctcacctgcgcccccgtatcgggcgcatgatgtaagagaaaccgaaaataaatgatactcaccaaggccatggcgaccagcgtgcgttcgtttcttctctgttgttgttgtatctagtatgatgaggcgtgcgtacccggagggtcctcctccctcgtacgagagcgtgatgcagcaggcgatggcggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggccagctgaaaaccatcatgcacaccaacatgcccaacgtgaacgagttcatgtacagcaacaagttcaaggcgcgggtgatggtctcccgcaagacccccaatggggtgacagtgacagaggattatgatggtagtcaggatgagctgaagtatgaatgggtggaatttgagctgcccgaaggcaacttctcggtgaccatgaccatcgacctgatgaacaacgccatcatcgacaattacttggcggtggggcggcagaacggggtgctggagagcgacatcggcgtgaagttcgacactaggaacttcaggctgggctgggaccccgtgaccgagctggtcatgcccggggtgtacaccaacgaggctttccatcccgatattgtcttgctgcccggctgcggggtggacttcaccgagagccgcctcagcaacctgctgggcattcgcaagaggcagcccttccaggaaggcttccagatcatgtacgaggatctggaggggggcaacatccccgcgctcctggatgtcgacgcctatgagaaaagcaaggaggatgcagcagctgaagcaactgcagccgtagctaccgcctctaccgaggtcaggggcgataattttgcaagcgccgcagcagtggcagcggccgaggcggctgaaaccgaaagtaagatagtcattcagccggtggagaaggatagcaagaacaggagctacaacgtactaccggacaagataaacaccgcctaccgcagctggtacctagcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgcttacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcggtcgcgcaccaccgtcgacgacgtgatcgaccaggtggtggccgacgcgcgcaactacacccccgccgccgcgcccgtctccaccgtggacgccgtcatcgacagcgtggtggccgacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcttcaggcgccagcgccggcaggacccggagacgcgcggccacggcggcggcagcggccatcgccagcatgtcccgcccgcggcgagggaacgtgtactgggtgcgcgacgccgccaccggtgtgcgcgtgcccgtgcgcacccgcccccctcgcacttgaagatgttcacttcgcgatgttgatgtgtcccagcggcgaggaggatgtccaagcgcaaattcaaggaagagatgctccaggtcatcgcgcctgagatctacggccctgcggtggtgaaggaggaaagaaagccccgcaaaatcaagcgggtcaaaaaggacaaaaaggaagaagaaagtgatgtggacggattggtggagtttgtgcgcgagttcgccccccggcggcgcgtgcagtggcgcgggcggaaggtgcaaccggtgctgagacccggcaccaccgtggtcttcacgcccggcgagcgctccggcaccgcttccaagcgctcctacgacgaggtgtacggggatgatgatattctggagcaggcggccgagcgcctgggcgagtttgcttacggcaagcgcagccgttccgcaccgaaggaagaggcggtgtccatcccgctggaccacggcaaccccacgccgagcctcaagcccgtgaccttgcagcaggtgctgccgaccgcggcgccgcgccgggggttcaagcgcgagggcgaggatctgtaccccaccatgcagctgatggtgcccaagcgccagaagctggaagacgtgctggagaccatgaaggtggacccggacgtgcagcccgaggtcaaggtgcggcccatcaagcaggtggccccgggcctgggcgtgcagaccgtggacatcaagattcccacggagcccatggaaacgcagaccgagcccatgatcaagcccagcaccagcaccatggaggtgcagacggatccctggatgccatcggctcctagtcgaagaccccggcgcaagtacggcgcggccagcctgctgatgcccaactacgcgctgcatccttccatcatccccacgccgggctaccgcggcacgcgcttctaccgcggtcataccagcagccgccgccgcaagaccaccactcgccgccgccgtcgccgcaccgccgctgcaaccacccctgccgccctggtgcggagagtgtaccgccgcggccgcgcacctctgaccctgccgcgcgcgcgctaccacccgagcatcgccatttaaactttcgcctgctttgcagatcaatggccctcacatgccgccttcgcgttcccattacgggctaccgaggaagaaaaccgcgccgtagaaggctggcggggaacgggatgcgtcgccaccaccaccggcggcggcgcgccatcagcaagcggttggggggaggcttcctgcccgcgctgatccccatcatcgccgcggcgatcggggcgatccccggcattgcttccgtggcggtgcaggcctctcagcgccactgagacacacttggaaacatcttgtaataaaccaatggactctgacgctcctggtcctgtgatgtgttttcgtagacagatggaagacatcaatttttcgtccctggctccgcgacacggcacgcggccgttcatgggcacctggagcgacatcggcaccagccaactgaacgggggcgccttcaattggagcagtctctggagcgggcttaagaatttcgggtccacgcttaaaacctatggcagcaaggcgtggaacagcaccacagggcaggcgctgagggataagctgaaagagcagaacttccagcagaaggtggtcgatgggctcgcctcgggcatcaacggggtggtggacctggccaaccaggccgtgcagcggcagatcaacagccgcctggacccggtgccgcccgccggctccgtggagatgccgcaggtggaggaggagctgcctcccctggacaagcggggcgagaagcgaccccgccccgatgcggaggagacgctgctgacgcacacggacgagccgcccccgtacgaggaggcggtgaaactgggtctgcccaccacgcggcccatcgcgcccctggccaccggggtgctgaaacccgaaaagcccgcgaccctggacttgcctcctccccagccttcccgcccctctacagtggctaagcccctgccgccggtggccgtggcccgcgcgcgacccgggggcaccgcccgccctcatgcgaactggcagagcactctgaacagcatcgtgggtctgggagtgcagagtgtgaagcgccgccgctgctattaaacctaccgtagcgcttaacttgcttgtctgtgtgtgtatgtattatgtcgccgccgccgctgtccaccagaaggaggagtgaagaggcgcgtcgccgagttgcaagatggccaccccatcgatgctgccccagtgggcgtacatgcacatcgccggacaggacgcttcggagtacctgagtccgggtctggtgcagtttgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccctagcttcaaaccctactccggcaccgcctacaacagtctggcccccaagggagcacccaacacttgtcagtggacatataaagccgatggtgaaactgccacagaaaaaacctatacatatggaaatgcacccgtgcagggcattaacatcacaaaagatggtattcaacttggaactgacaccgatgatcagccaatctacgcagataaaacctatcagcctgaacctcaagtgggtgatgctgaatggcatgacatcactggtactgatgaaaagtatggaggcagagctcttaagcctgataccaaaatgaagccttgttatggttcttttgccaagcctactaataaagaaggaggtcaggcaaatgtgaaaacaggaacaggcactactaaagaatatgacatagacatggctttctttgacaacagaagtgcggctgctgctggcctagctccagaaattgttttgtatactgaaaatgtggatttggaaactccagatacccatattgtatacaaagcaggcacagatgacagcagctcttctattaatttgggtcagcaagccatgcccaacagacctaactacattggtttcagagacaactttatcgggctcatgtactacaacagcactggcaatatgggggtgctggccggtcaggcttctcagctgaatgctgtggttgacttgcaagacagaaacaccgagctgtcctaccagctcttgcttgactctctgggtgacagaacccggtatttcagtatgtggaatcaggcggtggacagctatgatcctgatgtgcgcattattgaaaatcatggtgtggaggatgaacttcccaactattgtttccctctggatgctgttggcagaacagatacttatcagggaattaaggctaatggaactgatcaaaccacatggaccaaagatgacagtgtcaatgatgctaatgagataggcaagggtaatccattcgccatggaaatcaacatccaagccaacctgtggaggaacttcctctacgccaacgtggccctgtacctgcccgactcttacaagtacacgccggccaatgttaccctgcccaccaacaccaacacctacgattacatgaacggccgggtggtggcgccctcgctggtggactcctacatcaacatcggggcgcgctggtcgctggatcccatggacaacgtgaaccccttcaaccaccaccgcaatgcggggctgcgctaccgctccatgctcctgggcaacgggcgctacgtgcccttccacatccaggtgccccagaaatttttcgccatcaagagcctcctgctcctgcccgggtcctacacctacgagtggaacttccgcaaggacgtcaacatgatcctgcagagctccctcggcaacgacctgcgcacggacggggcctccatctccttcaccagcatcaacctctacgccaccttcttccccatggcgcacaacacggcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggctggtccttcacgcgtctcaagaccaaggagacgccctcgctgggctccgggttcgacccctacttcgtctactcgggctccatcccctacctcgacggcaccttctacctcaaccacaccttcaagaaggtctccatcaccttcgactcctccgtcagctggcccggcaacgaccggctcctgacgcccaacgagttcgaaatcaagcgcaccgtcgacggcgagggctacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtggtggacgaggtcaactacaaggactaccaggccgtcaccctggcctaccagcacaacaactcgggcttcgtcggctacctcgcgcccaccatgcgccagggccagccctaccccgccaactacccctacccgctcatcggcaagagcgccgtcaccagcgtcacccagaaaaagttcctctgcgacagggtcatgtggcgcatccccttctccagcaacttcatgtccatgggcgcgctcaccgacctcggccagaacatgctctatgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacccccttctcggccggtaacgccaccacctaagctcttgcttcttgcaagccatggccgcgggctccggcgagcaggagctcagggccatcatccgcgacctgggctgcgggccctacttcctgggcaccttcgataagcgcttcccgggattcatggccccgcacaagctggcctgcgccatcgtcaacacggccggccgcgagaccgggggcgagcactggctggccttcgcctggaacccgcgctcgaacacctgctacctcttcgaccccttcgggttctcggacgagcgcctcaagcagatctaccagttcgagtacgagggcctgctgcgccgcagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaactcccactccgcctactttcgctcccaccgcgcgcgcatcgagaaggccaccgccttcgaccgcatgaatcaagacatgtaaaccgtgtgtgtatgttaaatgtctttaataaacagcactttcatgttacacatgcatctgagatgatttatttagaaatcgaaagggttctgccgggtctcggcatggcccgcgggcagggacacgttgcggaactggtacttggccagccacttgaactcggggatcagcagtttgggcagcggggtgtcggggaaggagtcggtccacagcttccgcgtcagttgcagggcgcccagcaggtcgggcgcggagatcttgaaatcgcagttgggacccgcgttctgcgcgcgggagttgcggtacacggggttgcagcactggaacaccatcagggccgggtgcttcacgctcgccagcaccgtcgcgtcggtgatgctctccacgtcgaggtcctcggcgttggccatcccgaagggggtcatcttgcaggtctgccttcccatggtgggcacgcacccgggcttgtggttgcaatcgcagtgcagggggatcagcatcatctgggcctggtcggcgttcatccccgggtacatggccttcatgaaagcctccaattgcctgaacgcctgctgggccttggctccctcggtgaagaagaccccgcaggacttgctagagaactggttggtggcgcacccggcgtcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcatgtgctccttctggatcatggtggtcccgtgcaggcaccgcagcttgccctcggcctcggtgcacccgtgcagccacagcgcgcacccggtgcactcccagttcttgtgggcgatctgggaatgcgcgtgcacgaagccctgcaggaagcggcccatcatggtggtcagggtcttgttgctagtgaaggtcagcggaatgccgcggtgctcctcgttgatgtacaggtggcagatgcggcggtacacctcgccctgctcgggcatcagctggaagttggctttcaggtcggtctccacgcggtagcggtccatcagcatagtcatgatttccatacccttctcccaggccgagacgatgggcaggctcatagggttcttcaccatcatcttagcgctagcagccgcggccagggggtcgctctcgtccagggtctcaaagctccgcttgccgtccttctcggtgatccgcaccggggggtagctgaagcccacggccgccagctcctcctcggcctgtctttcgtcctcgctgtcctggctgacgtcctgcaggaccacatgcttggtcttgcggggtttcttcttgggcggcagcggcggcggagatgttggagatggcgagggggagcgcgagttctcgctcaccactactatctcttcctcttcttggtccgaggccacgcggcggtaggtatgtctcttcgggggcagaggcggaggcgacgggctctcgccgccgcgacttggcggatggctggcagagccccttccgcgttcgggggtgcgctcccggcggcgctctgactgacttcctccgcggccggccattgtgttctcctagggaggaacaacaagcatggagactcagccatcgccaacctcgccatctgcccccaccgccgacgagaagcagcagcagcagaatgaaagcttaaccgccccgccgcccagccccgccacctccgacgcggccgtcccagacatgcaagagatggaggaatccatcgagattgacctgggctatgtgacgcccgcggagcacgaggaggagctggcagtgcgcttttcacaagaagagatacaccaagaacagccagagcaggaagcagagaatgagcagagtcaggctgggctcgagcatgacggcgactacctccacctgagcgggggggaggacgcgctcatcaagcatctggcccggcaggccaccatcgtcaaggatgcgctgctcgaccgcaccgaggtgcccctcagcgtggaggagctcagccgcgcctacgagttgaacctcttctcgccgcgcgtgccccccaagcgccagcccaatggcacctgcgagcccaacccgcgcctcaacttctacccggtcttcgcggtgcccgaggccctggccacctaccacatctttttcaagaaccaaaagatccccgtctcctgccgcgccaaccgcacccgcgccgacgcccttttcaacctgggtcccggcgcccgcctacctgatatcgcctccttggaagaggttcccaagatcttcgagggtctgggcagcgacgagactcgggccgcgaacgctctgcaaggagaaggaggagagcatgagcaccacagcgccctggtcgagttggaaggcgacaacgcgcggctggcggtgctcaaacgcacggtcgagctgacccatttcgcctacccggctctgaacctgccccccaaagtcatgagcgcggtcatggaccaggtgctcatcaagcgcgcgtcgcccatctccgaggacgagggcatgcaagactccgaggagggcaagcccgtggtcagcgacgagcagctggcccggtggctgggtcctaatgctagtccccagagtttggaagagcggcgcaaactcatgatggccgtggtcctggtgaccgtggagctggagtgcctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcttgcacgagaaccgcctggggcagaacgtgctgcacaccaccctgcgcggggaggcccggcgcgactacatccgcgactgcgtctacctctacctctgccacacctggcagacgggcatgggcgtgtggcagcagtgtctggaggagcagaacctgaaagagctctgcaagctcctgcagaagaacctcaagggtctgtggaccgggttcgacgagcgcaccaccgcctcggacctggccgacctcattttccccgagcgcctcaggctgacgctgcgcaacggcctgcccgactttatgagccaaagcatgttgcaaaactttcgctctttcatcctcgaacgctccggaatcctgcccgccacctgctccgcgctgccctcggacttcgtgccgctgaccttccgcgagtgccccccgccgctgtggagccactgctacctgctgcgcctggccaactacctggcctaccactcggacgtgatcgaggacgtcagcggcgagggcctgctcgagtgccactgccgctgcaacctctgcacgccgcaccgctccctggcctgcaacccccagctgctgagcgagacccagatcatcggcaccttcgagttgcaagggcccagcgaaggcgagggttcagccgccaaggggggtctgaaactcaccccggggctgtggacctcggcctacttgcgcaagttcgtgcccgaggactaccatcccttcgagatcaggttctacgaggaccaatcccatccgcccaaggccgagctgtcggcctgcgtcatcacccagggggcgatcctggcccaattgcaagccatccagaaatcccgccaagaattcttgctgaaaaagggccgcggggtctacctcgacccccagaccggtgaggagctcaaccccggcttcccccaggatgccccgaggaaacaagaagctgaaagtggagctgccgcccgtggaggatttggaggaagactgggagaacagcagtcaggcagaggaggaggagatggaggaagactgggacagcactcaggcagaggaggacagcctgcaagacagtctggaggaagacgaggaggaggcagaggaggaggtggaagaagcagccgccgccagaccgtcgtcctcggcgggggagaaagcaagcagcacggataccatctccgctccgggtcggggtcccgctcgaccacacagtagatgggacgagaccggacgattcccgaaccccaccacccagaccggtaagaaggagcggcagggatacaagtcctggcgggggcacaaaaacgccatcgtctcctgcttgcaggcctgcgggggcaacatctccttcacccggcgctacctgctcttccaccgcggggtgaactttccccgcaacatcttgcattactaccgtcacctccacagcccctactacttccaagaagaggcagcagcagcagaaaaagaccagcagaaaaccagcagctagaaaatccacagcggcggcagcaggtggactgaggatcgcggcgaacgagccggcgcaaacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccagtcgcagaaaaaggcgggaattacgtcacctgtgcccttcgccctagccgcctccacccatcatcatgagcaaagagattcccacgccttacatgtggagctaccagccccagatgggcctggccgccggtgccgcccaggactactccacccgcatgaattggctcagcgccgggcccgcgatgatctcacgggtgaatgacatccgcgcccaccgaaaccagatactcctagaacagtcagcgctcaccgccacgccccgcaatcacctcaatccgcgtaattggcccgccgccctggtgtaccaggaaattccccagcccacgaccgtactacttccgcgagacgcccaggccgaagtccagctgactaactcaggtgtccagctggcgggcggcgccaccctgtgtcgtcaccgccccgctcagggtataaagcggctggtgatccggggcagaggcacacagctcaacgacgaggtggtgagctcttcgctgggtctgcgacctgacggagtcttccaactcgccggatcggggagatcttccttcacgcctcgtcaggccgtcctgactttggagagttcgtcctcgcagccccgctcgggtggcatcggcactctccagttcgtggaggagttcactccctcggtctacttcaaccccttctccggctcccccggccactacccggacgagttcatcccgaacttcgacgccatcagcgagtcggtggacggctacgattgaatgtcccatggtggcgcagctgacctagctcggcttcgacacctggaccactgccgccgcttccgctgcttcgctcgggatctcgccgagtttgcctactttgagctgcccgaggagcaccctcagggcccggcccacggagtgcggatcgtcgtcgaagggggcctcgactcccacctgcttcggatcttcagccagcgtccgatcctggtcgagcgcgagcaaggacagacccttctgactctgtactgcatctgcaaccaccccggcctgcatgaaagtctttgttgtctgctgtgtactgagtataataaaagctgagatcagcgactactccggacttccgtgtgttcctgaatccatcaaccagtctttgttcttcaccgggaacgagaccgagctccagctccagtgtaagccccacaagaagtacctcacctggctgttccagggctccccgatcgccgttgtcaaccactgcgacaacgacggagtcctgctgagcggccctgccaaccttactttttccacccgcagaagcaagctccagctcttccaacccttcctccccgggacctatcagtgcgtctcgggaccctgccatcacaccttccacctgatcccgaataccacagcgtcgctccccgctactaacaaccaaactaacctccaccaacgccaccgtcgcgacctttctgaatctaatactaccacccacaccggaggtgagctccgaggtcaaccaacctctgggatttactacggcccctgggaggtggttgggttaatagcgctaggcctagttgcgggtgggcttttggttctctgctacctatacctcccttgctgttcgtacttagtggtgctgtgttgctggtttaagaaatggggaagatcaccctagtgagctgcggtgcgctggtggcggtgttgctttcgattgtgggactgggcggtgcggctgtagtgaaggagaaggccgatccctgcttgcatttcaatcccaacaaatgccagctgagttttcagcccgatggcaatcggtgcgcggtactgatcaagtgcggatgggaatgcgagaacgtgagaatcgagtacaataacaagactcggaacaatactctcgcgtccgtgtggcagcccggggaccccgagtggtacaccgtctctgtccccggtgctgacggctccccgcgcaccgtgaataatactttcatttttgcgcacatgtgcgacacggtcatgtggatgagcaagcagtacgatatgtggccccccacgaaggagaacatcgtggtcttctccatcgcttacagcctgtgcacggcgctaatcaccgctatcgtgtgcctgagcattcacatgctcatcgctattcgccccagaaataatgccgaaaaagaaaaacagccataacgttttttttcacacctttttcagaccatggcctctgttaaatttttgcttttatttgccagtctcattgccgtcattcatggaatgagtaatgagaaaattactatttacactggcactaatcacacattgaaaggtccagaaaaagccacagaagtttcatggtattgttattttaatgaatcagatgtatctactgaactctgtggaaacaataacaaaaaaaatgagagcattactctcatcaagtttcaatgtggatctgacttaaccctaattaacatcactagagactatgtaggtatgtattatggaactacagcaggcatttcggacatggaattttatcaagtttctgtgtctgaacccaccacgcctagaatgaccacaaccacaaaaactacacctgttaccactatgcagctcactaccaataacatttttgccatgcgtcaaatggtcaacaatagcactcaacccaccccacccagtgaggaaattcccaaatccatgattggcattattgttgctgtagtggtgtgcatgttgatcatcgccttgtgcatggtgtactatgccttctgctacagaaagcacagactgaacgacaagctggaacacttactaagtgttgaattttaattttttagaaccatgaagatcctaggccttttaattttttctatcattacctctgctctatgcaattctgacaatgaggacgttactgtcgttgtcggatcaaattatacactgaaaggtccagcgaagggtatgctttcgtggtattgctattttggatctgacactacagaaactgaattatgcaatcttaagaatggcaaaattcaaaattctaaaattaacaattatatatgcaatggtactgatctgatactcctcaatatcacgaaatcatatgctggcagttacacctgccctggagatgatgctgacagtatgattttttacaaagtaactgttgttgatcccactactccacctccacccaccacaactactcacaccacacacacagatcaaaccgcagcagaggaggcagcaaagttagccttgcaggtccaagacagttcatttgttggcattacccctacacctgatcagcggtgtccggggctgctagtcagcggcattgtcggtgtgctttcgggattagcagtcataatcatctgcatgttcatttttgcttgctgctatagaaggctttaccgacaaaaatcagacccactgctgaacctctatgtttaattttttccagagtcatgaaggcagttagcgctctagttttttgttctttgattggcattgttttttgcaatcctattcctaaagttagctttattaaagatgtgaatgttactgaggggggcaatgtgacactggtaggtgtagagggtgctgaaaacaccacctggacaaaataccacctcaatgggtggaaagatatttgcaattggagtgtattagtttatacatgtgagggagttaatcttaccattgtcaatgccacctcagctcaaaatggtagaattcaaggacaaagtgtcagtgtatctaatgggtattttacccaacatacttttatctatgacgttaaagtcataccactgcctacgcctagcccacctagcactaccacacagacaacccacactacacagacaaccacatacagtacattaaatcagcctaccaccactacagcagcagaggttgccagctcgtctggggtccgagtggcatttttgatgtgggccccatctagcagtcccactgctagtaccaatgagcagactactgaatttttgtccactgtcgagagccacaccacagctacctccagtgccttctctagcaccgccaatctctcctcgctttcctctacaccaatcagtcccgctactactcctagccccgctcctcttcccactcccctgaagcaaacagacggcggcatgcaatggcagatcaccctgctcattgtgatcgggttggtcatcctggccgtgttgctctactacatcttctgccgccgcattcccaacgcgcaccgcaagccggtctacaagcccatcattgtcgggcagccggagccgcttcaggtggaagggggtctaaggaatcttctcttctcttttacagtatggtgattgaactatgattcctagacaattcttgatcactattcttatctgcctcctccaagtctgtgccaccctcgctctggtggccaacgccagtccagactgtattgggcccttcgcctcctacgtgctctttgccttcaccacctgcatctgctgctgtagcatagtctgcctgcttatcaccttcttccagttcattgactggatctttgtgcgcatcgcctacctgcgccaccacccccagtaccgcgaccagcgagtggcgcggctgctcaggctcctctgataagcatgcgggctctgctacttctcgcgcttctgctgttagtgctcccccgtcccgtcgacccccggtcccccacccagtcccccgaggaggtccgcaaatgcaaattccaagaaccctggaaattcctcaaatgctaccgccaaaaatcagacatgcatcccagctggatcatgatcattgggatcgtgaacattctggcctgcaccctcatctcctttgtgatttacccctgctttgactttggttggaactcgccagaggcgctctatctcccgcctgaacctgacacaccaccacagcaacctcaggcacacgcactaccaccactacagcctaggccacaatacatgcccatattagactatgaggccgagccacagcgacccatgctccccgctattagttacttcaatctaaccggcggagatgactgacccactggccaacaacaacgtcaacgaccttctcctggacatggacggccgcgcctcggagcagcgactcgcccaacttcgcattcgccagcagcaggagagagccgtcaaggagctgcaggatgcggtggccatccaccagtgcaagagaggcatcttctgcctggtgaaacaggccaagatctcctacgaggtcactccaaacgaccatcgcctctcctacgagctcctgcagcagcgccagaagttcacctgcctggtcggagtcaaccccatcgtcatcacccagcagtctggcgataccaaggggtgcatccactgctcctgcgactcccccgactgcgtccacactctgatcaagaccctctgcggcctccgcgacctcctccccatgaactaatcacccccttatccagtgaaataaagatcatattgatgatgattttacagaaataaaaaataatcatttgatttgaaataaagatacaatcatattgatgatttgagtttaacaaaaaaataaagaatcacttacttgaaatctgataccaggtctctgtccatgttttctgccaacaccacttcactcccctcttcccagctctggtactgcaggccccggcgggctgcaaacttcctccacacgctgaaggggatgtcaaattcctcctgtccctcaatcttcattttatcttctatcagatgtccaaaaagcgcgtccgggtggatgatgacttcgaccccgtctacccctacgatgcagacaacgcaccgaccgtgcccttcatcaacccccccttcgtctcttcagatggattccaagagaagcccctgggggtgttgtccctgcgactggccgaccccgtcaccaccaagaacggggaaatcaccctcaagctgggagagggggtggacctcgattcctcgggaaaactcatctccaacacggccaccaaggccgccgcccctctcagtttttccaacaacaccatttcccttaacatggatcaccccttttacactaaagatggaaaattatccttacaagtttctccaccattaaatatactgagaacaagcattctaaacacactagctttaggttttggatcaggtttaggactccgtggctctgccttggcagtacagttagtctctccacttacatttgatactgatggaaacataaagcttaccttagacagaggtttgcatgttacaacaggagatgcaattgaaagcaacataagctgggctaaaggtttaaaatttgaagatggagccatagcaaccaacattggaaatgggttagagtttggaagcagtagtacagaaacaggtgttgatgatgcttacccaatccaagttaaacttggatctggccttagctttgacagtacaggagccataatggctggtaacaaagaagacgataaactcactttgtggacaacacctgatccatcaccaaactgtcaaatactcgcagaaaatgatgcaaaactaacactttgcttgactaaatgtggtagtcaaatactggccactgtgtcagtcttagttgtaggaagtggaaacctaaaccccattactggcaccgtaagcagtgctcaggtgtttctacgttttgatgcaaacggtgttcttttaacagaacattctacactaaaaaaatactgggggtataggcagggagatagcatagatggcactccatataccaatgctgtaggattcatgcccaatttaaaagcttatccaaagtcacaaagttctactactaaaaataatatagtagggcaagtatacatgaatggagatgtttcaaaacctatgcttctcactataaccctcaatggtactgatgacagcaacagtacatattcaatgtcattttcatacacctggactaatggaagctatgttggagcaacatttggggctaactcttataccttctcatacatcgcccaagaatgaacactgtatcccaccctgcatgccaacccttcccaccccactctgtggaacaaactctgaaacacaaaataaaataaagttcaagtgttttattgattcaacagttttacaggattcgagcagttatttttcctccaccctcccaggacatggaatacaccaccctctccccccgcacagccttgaacatctgaatgccattggtgatggacatgcttttggtctccacgttccacacagtttcagagcgagccagtctcgggtcggtcagggagatgaaaccctccgggcactcccgcatctgcacctcacagctcaacagctgaggattgtcctcggtggtcgggatcacggttatctggaagaagcagaagagcggcggtgggaatcatagtccgcgaacgggatcggccggtggtgtcgcatcaggccccgcagcagtcgctgccgccgccgctccgtcaagctgctgctcagggggtccgggtccagggactccctcagcatgatgcccacggccctcagcatcagtcgtctggtgcggcgggcgcagcagcgcatgcggatctcgctcaggtcgctgcagtacgtgcaacacagaaccaccaggttgttcaacagtccatagttcaacacgctccagccgaaactcatcgcgggaaggatgctacccacgtggccgtcgtaccagatcctcaggtaaatcaagtggtgccccctccagaacacgctgcccacgtacatgatctccttgggcatgtggcggttcaccacctcccggtaccacatcaccctctggttgaacatgcagccccggatgatcctgcggaaccacagggccagcaccgccccgcccgccatgcagcgaagagaccccgggtcccggcaatggcaatggaggacccaccgctcgtacccgtggatcatctgggagctgaacaagtctatgttggcacagcacaggcatatgctcatgcatctcttcagcactctcaactcctcgggggtcaaaaccatatcccagggcacggggaactcttgcaggacagcgaaccccgcagaacagggcaatcctcgcacagaacttacattgtgcatggacagggtatcgcaatcaggcagcaccgggtgatcctccaccagagaagcgcgggtctcggtctcctcacagcgtggtaagggggccggccgatacgggtgatggcgggacgcggctgatcgtgttcgcgaccgtgtcatgatgcagttgctttcggacattttcgtacttgctgtagcagaacctggtccgggcgctgcacaccgatcgccggcggcggtctcggcgcttggaacgctcggtgttgaaattgtaaaacagccactctctcagaccgtgcagcagatctagggcctcaggagtgatgaagatcccatcatgcctgatggctctgatcacatcgaccaccgtggaatgggccagacccagccagatgatgcaattttgttgggtttcggtgacggcgggggagggaagaacaggaagaaccatgattaacttttaatccaaacggtctcggagtacttcaaaatgaagatcgcggagatggcacctctcgcccccgctgtgttggtggaaaataacagccaggtcaaaggtgatacggttctcgagatgttccacggtggcttccagcaaagcctccacgcgcacatccagaaacaagacaatagcgaaagcgggagggttctctaattcctcaatcatcatgttacactcctgcaccatccccagataattttcatttttccagccttgaatgattcgaactagttcctgaggtaaatccaagccagccatgataaagagctcgcgcagagcgccctccaccggcattcttaagcacaccctcataattccaagatattctgctcctggttcacctgcagcagattgacaagcggaatatcaaaatctctgccgcgatccctgagctcctccctcagcaataactgtaagtactctttcatatcctctccgaaatttttagccataggaccaccaggaataagattagggcaagccacagtacagataaaccgaagtcctccccagtgagcattgccaaatgcaagactgctataagcatgctggctagacccggtgatatcttccagataactggacagaaaatcgcccaggcaatttttaagaaaatcaacaaaagaaaaatcctccaggtggacgtttagagcctcgggaacaacgatgaagtaaatgcaagcggtgcgttccagcatggttagttagctgatctgtagaaaaaacaaaaatgaacattaaaccatgctagcctggcgaacaggtgggtaaatcgttctctccagcaccaggcaggccacggggtctccggcgcgaccctcgtaaaaattgtcgctatgattgaaaaccatcacagagagacgttcccggtggccggcgtgaatgattcgacaagatgaatacacccccggaacattggcgtccgcgagtgaaaaaaagcgcccgaggaagcaataaggcactacaatgctcagtctcaagtccagcaaagcgatgccatgcggatgaagcacaaaattctcaggtgcgtacaaaatgtaattactcccctcctgcacaggcagcaaagcccccgatccctccaggtacacatacaaagcctcagcgtccatagcttaccgagcagcagcacacaacaggcgcaagagtcagagaaaggctgagctctaacctgtccacccgctctctgctcaatatatagcccagatctacactgacgtaaaggccaaagtctaaaaatacccgccaaataatcacacacgcccagcacacgcccagaaaccggtgacacactcaaaaaaatacgcgcacttcctcaaacgcccaaaactgccgtcatttccgggttcccacgctacgtcatcaaaacacgactttcaaattccgtcgaccgttaaaaacgtcacccgccccgcccctaacggtcgcccgtctctcagccaatcagcgccccgcatccccaaattcaaacacctcatttgcatattaacgcgcacaaaaagtttgaggtatattattgatgatgg

SEQ ID NO: 2 - DNA, 31588 bp, synthetic construct
  misc_feature 1..31588: note = Description of Artificial Sequence: Synthetic polynucleotide
  source 1..31588: mol_type = other DNA
ccatcttcaataatatacctcaaactttttgtgcgcgttaatatgcaaatgaggcgtttgaatttggggaggaagggcggtgattggtcgagggatgagcgaccgttaggggcggggcgagtgacgttttgatgacgtggttgcgaggaggagccagtttgcaagttctcgtgggaaaagtgacgtcaaacgaggtgtggtttgaacacggaaatactcaattttcccgcgctctctgacaggaaatgaggtgtttctgggcggatgcaagtgaaaacgggccattttcgcgcgaaaactgaatgaggaagtgaaaatctgagtaatttcgcgtttatggcagggaggagtatttgccgagggccgagtagactttgaccgattacgtgggggtttcgattaccgtgtttttcacctaaatttccgcgtacggtgtcaaagtccggtgtttttacgtaggtgtcagctgatcgccagggtatttaaacctgcgctctccagtcaagaggccactcttgagtgccagcgagaagagttttctcctccgcgccgcgagtcagatctacactttgaaagtagggataacagggtaatgacattgattattgactagttgttaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggaacgccatccacgctgttttgacctccatagaagacagcgatcgcgccaccatggccgggatgttccaggcactgtccgaaggctgcacaccctatgatattaaccagatgctgaatgtcctgggagaccaccaggtctctggcctggagcagctggagagcatcatcaacttcgagaagctgaccgagtggacaagctccaatgtgatgcctatcctgtccccactgaccaagggcatcctgggcttcgtgtttaccctgacagtgccttctgagcggggcctgtcttgcatcagcgaggcagacgcaaccacaccagagtccgccaatctgggcgaggagatcctgtctcagctgtacctgtggccccgggtgacatatcactccccttcttacgcctatcaccagttcgagcggagagccaagtacaagagacacttcccaggctttggccagtctctgctgttcggctaccccgtgtacgtgttcggcgattgcgtgcagggcgactgggatgccatccggtttagatactgcgcaccacctggatatgcactgctgaggtgtaacgacaccaattattccgccctgctggcagtgggcgccctggagggccctcgcaatcaggattggctgggcgtgccaaggcagctggtgacacgcatgcaggccatccagaacgcaggcctgtgcaccctggtggcaatgctggaggagacaatcttctggctgcaggcctttctgatggccctgaccgacagcggccccaagacaaacatcatcgtggattcccagtacgtgatgggcatctccaagccttctttccaggagtttgtggactgggagaacgtgagcccagagctgaattccaccgatcagccattctggcaggcaggaatcctggcaaggaacctggtgcctatggtggccacagtgcagggccagaatctgaagtaccagggccagagcctggtcatcagcgcctccatcatcgtgtttaacctgctggagctggagggcgactatcgggacgatggcaacgtgtgggtgcacaccccactgagccccagaacactgaacgcctgggtgaaggccgtggaggagaagaagggcatcccagtgcacctggagctggcctccatgaccaatatggagctgatgtctagcatcgtgcaccagcaggtgaggacatacggacccgtgttcatgtgcctgggaggcctgctgaccatggtggcaggagccgtgtggctgacagtgcgggtgctggagctgttcagagccgcccagctggccaacgatgtggtgctgcagatcatggagctgtgcggagcagcctttcgccaggtgtgccacaccacagtgccatggcccaatgcctccctgacccccaagtggaacaatgagacaacacagcctcagatcgccaactgtagcgtgtacgacttcttcgtgtggctgcactactatagcgtgagggataccctgtggccccgcgtgacataccacatgaataagtacgcctatcacatgctggagaggcgcgccaagtataagagaggccctggcccaggcgcaaagtttgtggcagcatggaccctgaaggccgccgccggccccggccccggccagtatatcaaggctaacagtaagttcattggaatcacagagctgggacccggacctggataatgagtttaaactcccatttaaatgtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataactataacggtcctaaggtagcgagtgagtagtgttctggggcgggggaggacctgcatgagggccagaataactgaaatctgtgcttttctgtgtgttgcagcagcatgagcggaagcggctcctttgagggaggggtattcagcccttatctgacggggcgtctcccctcctgggcgggagtgcgtcagaatgtgatgggatccacggtggacggccggcccgtgcagcccgcgaactcttcaaccctgacctatgcaaccctgagctcttcgtcgttggacgcagctgccgccgcagctgctgcatctgccgccagcgccgtgcgcggaatggccatgggcgccggctactacggcactctggtggccaactcgagttccaccaataatcccgccagcctgaacgaggagaagctgttgctgctgatggcccagctcgaggccttgacccagcgcctgggcgagctgacccagcaggtggctcagctgcaggagcagacgcgggccgcggttgccacggtgaaatccaaataaaaaatgaatcaataaataaacggagacggttgttgattttaacacagagtctgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcatggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcctttgtgcccgcccaggttttccatgcactcatccatgatgatggcgatgggcccgtgggcggcggcctgggcaaagacgtttcgggggtcggacacatcatagttgtggtcctgggtgaggtcatcataggccattttaatgaatttggggcggagggtgccggactgggggacaaaggtaccctcgatcccgggggcgtagttcccctcacagatctgcatctcccaggctttgagctcggagggggggatcatgtccacctgcggggcgataaagaacacggtttccggggcgggggagatgagctgggccgaaagcaagttccggagcagctgggacttgccgcagccggtggggccgtagatgaccccgatgaccggctgcaggtggtagttgagggagagacagctgccgtcctcccggaggaggggggccacctcgttcatcatctcgcgcacgtgcatgttctcgcgcaccagttccgccaggaggcgctctccccccagggataggagctcctggagcgaggcgaagtttttcagcggcttgagtccgtcggccatgggcattttggagagggtttgttgcaagagttccaggcggtcccagagctcggtgatgtgctctacggcatctcgatccagcagacctcctcgtttcgcgggttgggacggctgcgggagtagggcaccagacgatgggcgtccagcgcagccagggtccggtccttccagggtcgcagcgtccgcgtcagggtggtctccgtcacggtgaaggggtgcgcgccgggctgggcgcttgcgagggtgcgcttcaggctcatccggctggtcgaaaaccgctcccgatcggcgccctgcgcgtcggccaggtagcaattgaccatgagttcgtagttgagcgcctcggccgcgtggcctttggcgcggagcttacctttggaagtctgcccgcaggcgggacagaggagggacttgagggcgtagagcttgggggcgaggaagacggactcgggggcgtaggcgtccgcgccgcagtgggcgcagacggtctcgcactccacgagccaggtgaggtcgggctggtcggggtcaaaaaccagtttcccgccgttctttttgatgcgtttcttacctttggtctccatgagctcgtgtccccgctgggtgacaaagaggctgtccgtgtccccgtagaccgactttatgggccggtcctcgagcggtgtgccgcggtcctcctcgtagaggaaccccgcccactccgagacgaaagcccgggtccaggccagcacgaaggaggccacgtgggacgggtagcggtcgttgtccaccagcgggtccaccttttccagggtatgcaaacacatgtccccctcgtccacatccaggaaggtgattggcttgtaagtgtaggccacgtgaccgggggtcccggccgggggggtataaaagggtgcgggtccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccggcggagatgcctttcaagagcccctcgtccatctggtcagaaaagacgatctttttgttgtcgagcttggtggcgaaggagccgtagagggcgttggagaggagcttggcgatggagcgcatggtctggtttttttccttgtcggcgcgctccttggcggcgatgttgagctgcacgtactcgcgcgccacgcacttccattcggggaagacggtggtcagctcgtcgggcacgattctgacctgccagccccgattatgcagggtgatgaggtccacactggtggccacctcgccgcgcaggggctcattagtccagcagaggcgtccgcccttgcgcgagcagaaggggggcagggggtccagcatgacctcgtcgggggggtcggcatcgatggtgaagatgccgggcaggaggtcggggtcaaagtagctgatggaagtggccagatcgtccagggcagcttgccattcgcgcacggccagcgcgcgctcgtagggactgaggggcgtgccccagggcatgggatgggtaagcgcggaggcgtacatgccgcagatgtcgtagacgtagaggggctcctcgaggatgccgatgtaggtggggtagcagcgccccccgcggatgctggcgcgcacgtagtcatacagctcgtgcgagggggcgaggagccccgggcccaggttggtgcgactgggcttttcggcgcggtagacgatctggcggaaaatggcatgcgagttggaggagatggtgggcctttggaagatgttgaagtgggcgtggggcagtccgaccgagtcgcggatgaagtgggcgtaggagtcttgcagcttggcgacgagctcggcggtgactaggacgtccagagcgcagtagtcgagggtctcctggatgatgtcatacttgagctgtcccttttgtttccacagctcgcggttgagaaggaactcttcgcggtccttccagtactcttcgagggggaacccgtcctgatctgcacggtaagagcctagcatgtagaactggttgacggccttgtaggcgcagcagcccttctccacggggagggcgtaggcctgggcggccttgcgcagggaggtgtgcgtgagggcgaaagtgtccctgaccatgaccttgaggaactggtgcttgaagtcgatatcgtcgcagcccccctgctcccagagctggaagtccgtgcgcttcttgtaggcggggttgggcaaagcgaaagtaacatcgttgaagaggatcttgcccgcgcggggcataaagttgcgagtgatgcggaaaggttggggcacctcggcccggttgttgatgacctgggcggcgagcacgatctcgtcgaagccgttgatgttgtggcccacgatgtagagttccacgaatcgcggacggcccttgacgtggggcagtttcttgagctcctcgtaggtgagctcgtcggggtcgctgagcccgtgctgctcgagcgcccagtcggcgagatgggggttggcgcggaggaaggaagtccagagatccacggccagggcggtttgcagacggtcccggtactgacggaactgctgcccgacggccattttttcgggggtgacgcagtagaaggtgcgggggtccccgtgccagcgatcccatttgagctggagggcgagatcgagggcgagctcgacgagccggtcgtccccggagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccattggtggcgacgtcgatggcttgcagggtcccgtgcccctggggtgtgaccaccgtcccccgtttcttcttgggcggctggggcgacgggggcggtgcctcttccatggttagaagcggcggcgaggacgcgcgccgggcggcaggggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcttgaaggtctccgcggccggcgcgctccacggtggccgcgaggtcgttggagatgcggcccatgagctgcgagaaggcgttcatgcccgcctcgttccagacgcggctgtagaccacgacgccctcgggatcgcgggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaaacgttccatggcctcgtaaaagtccacggcgaagttgaaaaactgggagttgcgcgccgagacggtcaactcctcctccagaagacggatgagctcggcgatggtggcgcgcacctcgcgctcgaaggcccccgggagttcctccacttcctcttcttcctcctccactaacatctcttctacttcctcctcaggcggcagtggtggcgggggagggggcctgcgtcgccggcggcgcacgggcagacggtcgatgaagcgctcgatggtctcgccgcgccggcgtcgcatggtctcggtgacggcgcgcccgtcctcgcggggccgcagcgtgaagacgccgccgcgcatctccaggtggccgggggggtccccgttgggcagggagagggcgctgacgatgcatcttatcaattgccccgtagggactccgcgcaaggacctgagcgtctcgagatccacgggatctgaaaaccgctgaacgaaggcttcgagccagtcgcagtcgcaaggtaggctgagcacggtttcttctggcgggtcatgttggttgggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccaggtccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcttgctggatctgggtgagggtggtctggaagtcatcaaagtcgacgaagcggtggtaggctccggtgttgatggtgtaggagcagttggccatgacggaccagttgacggtctggtggcccggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtattggcactcccgtctcgacccaagcctgcaccaaccctccaggatacggaggcgggtcgttttgcaacttttttttggaggccggatgagactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagaccccatagccagccgacttctccagttacggagcgagcccctcttttgttttgtttgtttttgccagatgcatcccgtactgcggcagatgcgcccccaccaccctccaccgcaacaacagccccctccacagccggcgcttctgcccccgccccagcagcaacttccagccacgaccgccgcggccgccgtgagcggggctggacagagttatgatcaccagctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaagcgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagtttgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgtgcggtgagcgccagcaggcggcgcgagctgagcgaccaggagctgatgcatagtctgcagcgggccctgaccggggccgggaccgagggggagagctactttgacatgggcgcggacctgcactggcagcccagccgccgggccttggaggcggcggcaggaccctacgtagaagaggtggacgatgaggtggacgaggagggcgagtacctggaagactgatggcgcgaccgtatttttgctagatgcaacaacaacagccacctcctgatcccgcgatgcgggcggcgctgcagagccagccgtccggcattaactcctcggacgattggacccaggccatgcaacgcatcatggcgctgacgacccgcaaccccgaagcctttagacagcagccccaggccaaccggctctcggccatcctggaggccgtggtgccctcgcgctccaaccccacgcacgagaaggtcctggccatcgtgaacgcgctggtggagaacaaggccatccgcggcgacgaggccggcctggtgtacaacgcgctgctggagcgcgtggcccgctacaacagcaccaacgtgcagaccaacctggaccgcatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggctttcaagaacttgcagggcctgtggggcgtgcaggccccggtcggggaccgcgcgacggtgtcgagcctgctgacgccgaactcgcgcctgctgctgctgctggtggcccccttcacggacagcggcagcatcaaccgcaactcgtacctgggctacctgattaacctgtaccgcgaggccatcggccaggcgcacgtggacgagcagacctaccaggagatcacccacgtgagccgcgccctgggccaggacgacccgggcaacctggaagccaccctgaactttttgctgaccaaccggtcgcagaagatcccgccccagtacgcgctcagcaccgaggaggagcgcatcctgcgttacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctgaatccccactggctcccgccgccggggttctacacgggcgagtacgacatgcccgaccccaatgacgggttcctgtgggacgatgtggacagcagcgtgttctccccccgaccgggtgctaacgagcgccccttgtggaagaaggaaggcagcgaccgacgcccgtcctcggcgctgtccggccgcgagggtgctgccgcggcggtgcccgaggccgccagtcctttcccgagcttgcccttctcgctgaacagtatccgcagcagcgagctgggcaggatcacgcgcccgcgcttgctgggcgaagaggagtacttgaatgactcgctgttgagacccgagcgggagaagaacttccccaataacgggatagaaagcctggtggacaagatgagccgctggaagacgtatgcgcaggagcacagggacgatccccgggcgtcgcagggggccacgagccggggcagcgccgcccgtaaacgccggtggcacgacaggcagcggggacagatgtgggacgatgaggactccgccgacgacagcagcgtgttggacttgggtgggagtggtaacccgttcgctcacctgcgcccccgtatcgggcgcatgatgtaagagaaaccgaaaataaatgatactcaccaaggccatggcgaccagcgtgcgttcgtttcttctctgttgttgttgtatctagtatgatgaggcgtgcgtacccggagggtcctcctccctcgtacgagagcgtgatgcagcaggcgatggcggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggccagctgaaaaccatcatgcacaccaacatgcccaacgtgaacgagttcatgtacagcaacaagttcaaggcgcgggtgatggtctcccgcaagacccccaatggggtgacagtgacagaggattatgatggtagtcaggatgagctgaagtatgaatgggtggaatttgagctgcccgaaggcaacttctcggtgaccatgaccatcgacctgatgaacaacgccatcatcgacaattacttggcggtggggcggcagaacggggtgctggagagcgacatcggcgtgaagttcgacactaggaacttcaggctgggctgggaccccgtgaccgagctggtcatgcccggggtgtacaccaacgaggctttccatcccgatattgtcttgctgcccggctgcggggtggacttcaccgagagccgcctcagcaacctgctgggcattcgcaagaggcagcccttccaggaaggcttccagatcatgtacgaggatctggaggggggcaacatccccgcgctcctggatgtcgacgcctatgagaaaagcaaggaggatgcagcagctgaagcaactgcagccgtagctaccgcctctaccgaggtcaggggcgataattttgcaagcgccgcagcagtggcagcggccgaggcggctgaaaccgaaagtaagatagtcattcagccggtggagaaggatagcaagaacaggagctacaacgtactaccggacaagataaacaccgcctaccgcagctggtacctagcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgcttacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcggtcgcgcaccaccgtcgacgacgtgatcgaccaggtggtggccgacgcgcgcaactacacccccgccgccgcgcccgtctccaccgtggacgccgtcatcgacagcgtggtggccgacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcttcaggcgccagcgccggcaggacccggagacgcgcggccacggcggcggcagcggccatcgccagcatgtcccgcccgcggcgagggaacgtgtactgggtgcgcgacgccgccaccggtgtgcgcgtgcccgtgcgcacccgcccccctcgcacttgaagatgttcacttcgcgatgttgatgtgtcccagcggcgaggaggatgtccaagcgcaaattcaaggaagagatgctccaggtcatcgcgcctgagatctacggccctgcggtggtgaaggaggaaagaaagccccgcaaaatcaagcgggtcaaaaaggacaaaaaggaagaagaaagtgatgtggacggattggtggagtttgtgcgcgagttcgccccccggcggcgcgtgcagtggcgcgggcggaaggtgcaaccggtgctgagacccggcaccaccgtggtcttcacgcccggcgagcgctccggcaccgcttccaagcgctcctacgacgaggtgtacggggatgatgatattctggagcaggcggccgagcgcctgggcgagtttgcttacggcaagcgcagccgttccgcaccgaaggaagaggcggtgtccatcccgctggaccacggcaaccccacgccgagcctcaagcccgtgaccttgcagcaggtgctgccgaccgcggcgccgcgccgggggttcaagcgcgagggcgaggatctgtaccccaccatgcagctgatggtgcccaagcgccagaagctggaagacgtgctggagaccatgaaggtggacccggacgtgcagcccgaggtcaaggtgcggcccatcaagcaggtggccccgggcctgggcgtgcagaccgtggacatcaagattcccacggagcccatggaaacgcagaccgagcccatgatcaagcccagcaccagcaccatggaggtgcagacggatccctggatgccatcggctcctagtcgaagaccccggcgcaagtacggcgcggccagcctgctgatgcccaactacgcgctgcatccttccatcatccccacgccgggctaccgcggcacgcgcttctaccgcggtcataccagcagccgccgccgcaagaccaccactcgccgccgccgtcgccgcaccgccgctgcaaccacccctgccgccctggtgcggagagtgtaccgccgcggccgcgcacctctgaccctgccgcgcgcgcgctaccacccgagcatcgccatttaaactttcgcctgctttgcagatcaatggccctcacatgccgccttcgcgttcccattacgggctaccgaggaagaaaaccgcgccgtagaaggctggcggggaacgggatgcgtcgccaccaccaccggcggcggcgcgccatcagcaagcggttggggggaggcttcctgcccgcgctgatccccatcatcgccgcggcgatcggggcgatccccggcattgcttccgtggcggtgcaggcctctcagcgccactgagacacacttggaaacatcttgtaataaaccaatggactctgacgctcctggtcctgtgatgtgttttcgtagacagatggaagacatcaatttttcgtccctggctccgcgacacggcacgcggccgttcatgggcacctggagcgacatcggcaccagccaactgaacgggggcgccttcaattggagcagtctctggagcgggcttaagaatttcgggtccacgcttaaaacctatggcagcaaggcgtggaacagcaccacagggcaggcgctgagggataagctgaaagagcagaacttccagcagaaggtggtcgatgggctcgcctcgggcatcaacggggtggtggacctggccaaccaggccgtgcagcggcagatcaacagccgcctggacccggtgccgcccgccggctccgtggagatgccgcaggtggaggaggagctgcctcccctggacaagcggggcgagaagcgaccccgccccgatgcggaggagacgctgctgacgcacacggacgagccgcccccgtacgaggaggcggtgaaactgggtctgcccaccacgcggcccatcgcgcccctggccaccggggtgctgaaacccgaaaagcccgcgaccctggacttgcctcctccccagccttcccgcccctctacagtggctaagcccctgccgccggtggccgtggcccgcgcgcgacccgggggcaccgcccgccctcatgcgaactggcagagcactctgaacagcatcgtgggtctgggagtgcagagtgtgaagcgccgccgctgctattaaacctaccgtagcgcttaacttgcttgtctgtgtgtgtatgtattatgtcgccgccgccgctgtccaccagaaggaggagtgaagaggcgcgtcgccgagttgcaagatggccaccccatcgatgctgccccagtgggcgtacatgcacatcgccggacaggacgcttcggagtacctgagtccgggtctggtgcagtttgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccctagcttcaaaccctactccggcaccgcctacaacagtctggcccccaagggagcacccaacacttgtcagtggacatataaagccgatggtgaaactgccacagaaaaaacctatacatatggaaatgcacccgtgcagggcattaacatcacaaaagatggtattcaacttggaactgacaccgatgatcagccaatctacgcagataaaacctatcagcctgaacctcaagtgggtgatgctgaatggcatgacatcactggtactgatgaaaagtatggaggcagagctcttaagcctgataccaaaatgaagccttgttatggttcttttgccaagcctactaataaagaaggaggtcaggcaaatgtgaaaacaggaacaggcactactaaagaatatgacatagacatggctttctttgacaacagaagtgcggctgctgctggcctagctccagaaattgttttgtatactgaaaatgtggatttggaaactccagatacccatattgtatacaaagcaggcacagatgacagcagctcttctattaatttgggtcagcaagccatgcccaacagacctaactacattggtttcagagacaactttatcgggctcatgtactacaacagcactggcaatatgggggtgctggccggtcaggcttctcagctgaatgctgtggttgacttgcaagacagaaacaccgagctgtcctaccagctcttgcttgactctctgggtgacagaacccggtatttcagtatgtggaatcaggcggtggacagctatgatcctgatgtgcgcattattgaaaatcatggtgtggaggatgaacttcccaactattgtttccctctggatgctgttggcagaacagatacttatcagggaattaaggctaatggaactgatcaaaccacatggaccaaagatgacagtgtcaatgatgctaatgagataggcaagggtaatccattcgccatggaaatcaacatccaagccaacctgtggaggaacttcctctacgccaacgtggccctgtacctgcccgactcttacaagtacacgccggccaatgttaccctgcccaccaacaccaacacctacgattacatgaacggccgggtggtggcgccctcgctggtggactcctacatcaacatcggggcgcgctggtcgctggatcccatggacaacgtgaaccccttcaaccaccaccgcaatgcggggctgcgctaccgctccatgctcctgggcaacgggcgctacgtgcccttccacatccaggtgccccagaaatttttcgccatcaagagcctcctgctcctgcccgggtcctacacctacgagtggaacttccgcaaggacgtcaacatgatcctgcagagctccctcggcaacgacctgcgcacggacggggcctccatctccttcaccagcatcaacctctacgccaccttcttccccatggcgcacaacacggcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggctggtccttcacgcgtctcaagaccaaggagacgccctcgctgggctccgggttcgacccctacttcgtctactcgggctccatcccctacctcgacggcaccttctacctcaaccacaccttcaagaaggtctccatcaccttcgactcctccgtcagctggcccggcaacgaccggctcctgacgcccaacgagttcgaaatcaagcgcaccgtcgacggcgagggctacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtggtggacgaggtcaactacaaggactaccaggccgtcaccctggcctaccagcacaacaactcgggcttcgtcggctacctcgcgcccaccatgcgccagggccagccctaccccgccaactacccctacccgctcatcggcaagagcgccgtcaccagcgtcacccagaaaaagttcctctgcgacagggtcatgtggcgcatccccttctccagcaacttcatgtccatgggcgcgctcaccgacctcggccagaacatgctctatgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacccccttctcggccggtaacgccaccacctaagctcttgcttcttgcaagccatggccgcgggctccggcgagcaggagctcagggccatcatccgcgacctgggctgcgggccctacttcctgggcaccttcgataagcgcttcccgggattcatggccccgcacaagctggcctgcgccatcgtcaacacggccggccgcgagaccgggggcgagcactggctggccttcgcctggaacccgcgctcgaacacctgctacctcttcgaccccttcgggttctcggacgagcgcctcaagcagatctaccagttcgagtacgagggcctgctgcgccgcagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaactcccactccgcctactttcgctcccaccgcgcgcgcatcgagaaggccaccgccttcgaccgcatgaatcaagacatgtaaaccgtgtgtgtatgttaaatgtctttaataaacagcactttcatgttacacatgcatctgagatgatttatttagaaatcgaaagggttctgccgggtctcggcatggcccgcgggcagggacacgttgcggaactggtacttggccagccacttgaactcggggatcagcagtttgggcagcggggtgtcggggaaggagtcggtccacagcttccgcgtcagttgcagggcgcccagcaggtcgggcgcggagatcttgaaatcgcagttgggacccgcgttctgcgcgcgggagttgcggtacacggggttgcagcactggaacaccatcagggccgggtgcttcacgctcgccagcaccgtcgcgtcggtgatgctctccacgtcgaggtcctcggcgttggccatcccgaagggggtcatcttgcaggtctgccttcccatggtgggcacgcacccgggcttgtggttgcaatcgcagtgcagggggatcagcatcatctgggcctggtcggcgttcatccccgggtacatggccttcatgaaagcctccaattgcctgaacgcctgctgggccttggctccctcggtgaagaagaccccgcaggacttgctagagaactggttggtggcgcacccggcgtcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcatgtgctccttctggatcatggtggtcccgtgcaggcaccgcagcttgccctcggcctcggtgcacccgtgcagccacagcgcgcacccggtgcactcccagttcttgtgggcgatctgggaatgcgcgtgcacgaagccctgcaggaagcggcccatcatggtggtcagggtcttgttgctagtgaaggtcagcggaatgccgcggtgctcctcgttgatgtacaggtggcagatgcggcggtacacctcgccctgctcgggcatcagctggaagttggctttcaggtcggtctccacgcggtagcggtccatcagcatagtcatgatttccatacccttctcccaggccgagacgatgggcaggctcatagggttcttcaccatcatcttagcgctagcagccgcggccagggggtcgctctcgtccagggtctcaaagctccgcttgccgtccttctcggtgatccgcaccggggggtagctgaagcccacggccgccagctcctcctcggcctgtctttcgtcctcgctgtcctggctgacgtcctgcaggaccacatgcttggtcttgcggggtttcttcttgggcggcagcggcggcggagatgttggagatggcgagggggagcgcgagttctcgctcaccactactatctcttcctcttcttggtccgaggccacgcggcggtaggtatgtctcttcgggggcagaggcggaggcgacgggctctcgccgccgcgacttggcggatggctggcagagccccttccgcgttcgggggtgcgctcccggcggcgctctgactgacttcctccgcggccggccattgtgttctcctagggaggaacaacaagcatggagactcagccatcgccaacctcgccatctgcccccaccgccgacgagaagcagcagcagcagaatgaaagcttaaccgccccgccgcccagccccgccacctccgacgcggccgtcccagacatgcaagagatggaggaatccatcgagattgacctgggctatgtgacgcccgcggagcacgaggaggagctggcagtgcgcttttcacaagaagagatacaccaagaacagccagagcaggaagcagagaatgagcagagtcaggctgggctcgagcatgacggcgactacctccacctgagcgggggggaggacgcgctcatcaagcatctggcccggcaggccaccatcgtcaaggatgcgctgctcgaccgcaccgaggtgcccctcagcgtggaggagctcagccgcgcctacgagttgaacctcttctcgccgcgcgtgccccccaagcgccagcccaatggcacctgcgagcccaacccgcgcctcaacttctacccggtcttcgcggtgcccgaggccctggccacctaccacatctttttcaagaaccaaaagatccccgtctcctgccgcgccaaccgcacccgcgccgacgcccttttcaacctgggtcccggcgcccgcctacctgatatcgcctccttggaagaggttcccaagatcttcgagggtctgggcagcgacgagactcgggccgcgaacgctctgcaaggagaaggaggagagcatgagcaccacagcgccctggtcgagttggaaggcgacaacgcgcggctggcggtgctcaaacgcacggtcgagctgacccatttcgcctacccggctctgaacctgccccccaaagtcatgagcgcggtcatggaccaggtgctcatcaagcgcgcgtcgcccatctccgaggacgagggcatgcaagactccgaggagggcaagcccgtggtcagcgacgagcagctggcccggtggctgggtcctaatgctagtccccagagtttggaagagcggcgcaaactcatgatggccgtggtcctggtgaccgtggagctggagtgcctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcttgcacgagaaccgcctggggcagaacgtgctgcacaccaccctgcgcggggaggcccggcgcgactacatccgcgactgcgtctacctctacctctgccacacctggcagacgggcatgggcgtgtggcagcagtgtctggaggagcagaacctgaaagagctctgcaagctcctgcagaagaacctcaagggtctgtggaccgggttcgacgagcgcaccaccgcctcggacctggccgacctcattttccccgagcgcctcaggctgacgctgcgcaacggcctgcccgactttatgagccaaagcatgttgcaaaactttcgctctttcatcctcgaacgctccggaatcctgcccgccacctgctccgcgctgccctcggacttcgtgccgctgaccttccgcgagtgccccccgccgctgtggagccactgctacctgctgcgcctggccaactacctggcctaccactcggacgtgatcgaggacgtcagcggcgagggcctgctcgagtgccactgccgctgcaacctctgcacgccgcaccgctccctggcctgcaacccccagctgctgagcgagacccagatcatcggcaccttcgagttgcaagggcccagcgaaggcgagggttcagccgccaaggggggtctgaaactcaccccggggctgtggacctcggcctacttgcgcaagttcgtgcccgaggactaccatcccttcgagatcaggttctacgaggaccaatcccatccgcccaaggccgagctgtcggcctgcgtcatcacccagggggcgatcctggcccaattgcaagccatccagaaatcccgccaagaattcttgctgaaaaagggccgcggggtctacctcgacccccagaccggtgaggagctcaaccccggcttcccccaggatgccccgaggaaacaagaagctgaaagtggagctgccgcccgtggaggatttggaggaagactgggagaacagcagtcaggcagaggaggaggagatggaggaagactgggacagcactcaggcagaggaggacagcctgcaagacagtctggaggaagacgaggaggaggcagaggaggaggtggaagaagcagccgccgccagaccgtcgtcctcggcgggggagaaagcaagcagcacggataccatctccgctccgggtcggggtcccgctcgaccacacagtagatgggacgagaccggacgattcccgaaccccaccacccagaccggtaagaaggagcggcagggatacaagtcctggcgggggcacaaaaacgccatcgtctcctgcttgcaggcctgcgggggcaacatctccttcacccggcgctacctgctcttccaccgcggggtgaactttccccgcaacatcttgcattactaccgtcacctccacagcccctactacttccaagaagaggcagcagcagcagaaaaagaccagcagaaaaccagcagctagaaaatccacagcggcggcagcaggtggactgaggatcgcggcgaacgagccggcgcaaacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccagtcgcagaaaaaggcgggaattacgtcacctgtgcccttcgccctagccgcctccacccatcatcatgagcaaagagattcccacgccttacatgtggagctaccagccccagatgggcctggccgccggtgccgcccaggactactccacccgcatgaattggctcagcgccgggcccgcgatgatctcacgggtgaatgacatccgcgcccaccgaaaccagatactcctagaacagtcagcgctcaccgccacgccccgcaatcacctcaatccgcgtaattggcccgccgccctggtgtaccaggaaattccccagcccacgaccgtactacttccgcgagacgcccaggccgaagtccagctgactaactcaggtgtccagctggcgggcggcgccaccctgtgtcgtcaccgccccgctcagggtataaagcggctggtgatccggggcagaggcacacagctcaacgacgaggtggtgagctcttcgctgggtctgcgacctgacggagtcttccaactcgccggatcggggagatcttccttcacgcctcgtcaggccgtcctgactttggagagttcgtcctcgcagccccgctcgggtggcatcggcactctccagttcgtggaggagttcactccctcggtctacttcaaccccttctccggctcccccggccactacccggacgagttcatcccgaacttcgacgccatcagcgagtcggtggacggctacgattgaaactaatcacccccttatccagtgaaataaagatcatattgatgatgattttacagaaataaaaaataatcatttgatttgaaataaagatacaatcatattgatgatttgagtttaacaaaaaaataaagaatcacttacttgaaatctgataccaggtctctgtccatgttttctgccaacaccacttcactcccctcttcccagctctggtactgcaggccccggcgggctgcaaacttcctccacacgctgaaggggatgtcaaattcctcctgtccctcaatcttcattttatcttctatcagatgtccaaaaagcgcgtccgggtggatgatgacttcgaccccgtctacccctacgatgcagacaacgcaccgaccgtgcccttcatcaacccccccttcgtctcttcagatggattccaagagaagcccctgggggtgttgtccctgcgactggccgaccccgtcaccaccaagaacggggaaatcaccctcaagctgggagagggggtggacctcgattcctcgggaaaactcatctccaacacggccaccaaggccgccgcccctctcagtttttccaacaacaccatttcccttaacatggatcaccccttttacactaaagatggaaaattatccttacaagtttctccaccattaaatatactgagaacaagcattctaaacacactagctttaggttttggatcaggtttaggactccgtggctctgccttggcagtacagttagtctctccacttacatttgatactgatggaaacataaagcttaccttagacagaggtttgcatgttacaacaggagatgcaattgaaagcaacataagctgggctaaaggtttaaaatttgaagatggagccatagcaaccaacattggaaatgggttagagtttggaagcagtagtacagaaacaggtgttgatgatgcttacccaatccaagttaaacttggatctggccttagctttgacagtacaggagccataatggctggtaacaaagaagacgataaactcactttgtggacaacacctgatccatcaccaaactgtcaaatactcgcagaaaatgatgcaaaactaacactttgcttgactaaatgtggtagtcaaatactggccactgtgtcagtcttagttgtaggaagtggaaacctaaaccccattactggcaccgtaagcagtgctcaggtgtttctacgttttgatgcaaacggtgttcttttaacagaacattctacactaaaaaaatactgggggtataggcagggagatagcatagatggcactccatataccaatgctgtaggattcatgcccaatttaaaagcttatccaaagtcacaaagttctactactaaaaataatatagtagggcaagtatacatgaatggagatgtttcaaaacctatgcttctcactataaccctcaatggtactgatgacagcaacagtacatattcaatgtcattttcatacacctggactaatggaagctatgttggagcaacatttggggctaactcttataccttctcatacatcgcccaagaatgaacactgtatcccaccctgcatgccaacccttcccaccccactctgtggaacaaactctgaaacacaaaataaaataaagttcaagtgttttattgattcaacagttttacaggattcgagcagttatttttcctccaccctcccaggacatggaatacaccaccctctccccccgcacagccttgaacatctgaatgccattggtgatggacatgcttttggtctccacgttccacacagtttcagagcgagccagtctcgggtcggtcagggagatgaaaccctccgggcactcccgcatctgcacctcacagctcaacagctgaggattgtcctcggtggtcgggatcacggttatctggaagaagcagaagagcggcggtgggaatcatagtccgcgaacgggatcggccggtggtgtcgcatcaggccccgcagcagtcgctgccgccgccgctccgtcaagctgctgctcagggggtccgggtccagggactccctcagcatgatgcccacggccctcagcatcagtcgtctggtgcggcgggcgcagcagcgcatgcggatctcgctcaggtcgctgcagtacgtgcaacacagaaccaccaggttgttcaacagtccatagttcaacacgctccagccgaaactcatcgcgggaaggatgctacccacgtggccgtcgtaccagatcctcaggtaaatcaagtggtgccccctccagaacacgctgcccacgtacatgatctccttgggcatgtggcggttcaccacctcccggtaccacatcaccctctggttgaacatgcagccccggatgatcctgcggaaccacagggccagcaccgccccgcccgccatgcagcgaagagaccccgggtcccggcaatggcaatggaggacccaccgctcgtacccgtggatcatctgggagctgaacaagtctatgttggcacagcacaggcatatgctcatgcatctcttcagcactctcaactcctcgggggtcaaaaccatatcccagggcacggggaactcttgcaggacagcgaaccccgcagaacagggcaatcctcgcacagaacttacattgtgcatggacagggtatcgcaatcaggcagcaccgggtgatcctccaccagagaagcgcgggtctcggtctcctcacagcgtggtaagggggccggccgatacgggtgatggcgggacgcggctgatcgtgttcgcgaccgtgtcatgatgcagttgctttcggacattttcgtacttgctgtagcagaacctggtccgggcgctgcacaccgatcgccggcggcggtctcggcgcttggaacgctcggtgttgaaattgtaaaacagccactctctcagaccgtgcagcagatctagggcctcaggagtgatgaagatcccatcatgcctgatggctctgatcacatcgaccaccgtggaatgggccagacccagccagatgatgcaattttgttgggtttcggtgacggcgggggagggaagaacaggaagaaccatgattaacttttaatccaaacggtctcggagtacttcaaaatgaagatcgcggagatggcacctctcgcccccgctgtgttggtggaaaataacagccaggtcaaaggtgatacggttctcgagatgttccacggtggcttccagcaaagcctccacgcgcacatccagaaacaagacaatagcgaaagcgggagggttctctaattcctcaatcatcatgttacactcctgcaccatccccagataattttcatttttccagccttgaatgattcgaactagttcctgaggtaaatccaagccagccatgataaagagctcgcgcagagcgccctccaccggcattcttaagcacaccctcataattccaagatattctgctcctggttcacctgcagcagattgacaagcggaatatcaaaatctctgccgcgatccctgagctcctccctcagcaataactgtaagtactctttcatatcctctccgaaatttttagccataggaccaccaggaataagattagggcaagccacagtacagataaaccgaagtcctccccagtgagcattgccaaatgcaagactgctataagcatgctggctagacccggtgatatcttccagataactggacagaaaatcgcccaggcaatttttaagaaaatcaacaaaagaaaaatcctccaggtggacgtttagagcctcgggaacaacgatgaagtaaatgcaagcggtgcgttccagcatggttagttagctgatctgtagaaaaaacaaaaatgaacattaaaccatgctagcctggcgaacaggtgggtaaatcgttctctccagcaccaggcaggccacggggtctccggcgcgaccctcgtaaaaattgtcgctatgattgaaaaccatcacagagagacgttcccggtggccggcgtgaatgattcgacaagatgaatacacccccggaacattggcgtccgcgagtgaaaaaaagcgcccgaggaagcaataaggcactacaatgctcagtctcaagtccagcaaagcgatgccatgcggatgaagcacaaaattctcaggtgcgtacaaaatgtaattactcccctcctgcacaggcagcaaagcccccgatccctccaggtacacatacaaagcctcagcgtccatagcttaccgagcagcagcacacaacaggcgcaagagtcagagaaaggctgagctctaacctgtccacccgctctctgctcaatatatagcccagatctacactgacgtaaaggccaaagtctaaaaatacccgccaaataatcacacacgcccagcacacgcccagaaaccggtgacacactcaaaaaaatacgcgcacttcctcaaacgcccaaaactgccgtcatttccgggttcccacgctacgtcatcaaaacacgactttcaaattccgtcgaccgttaaaaacgtcacccgccccgcccctaacggtcgcccgtctctcagccaatcagcgccccgcatccccaaattcaaacacctcatttgcatattaacgcgcacaaaaagtttgagg

SEQ ID NO: 3 - DNA, 11447 bp, Venezuelan equine encephalitis virus
  source 1..11447: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgttcccgttccagccaatgtatccgatgcagccaatgccctatcgcaacccgttcgcggccccgcgcaggccctggttccccagaaccgacccttttctggcgatgcaggtgcaggaattaacccgctcgatggctaacctgacgttcaagcaacgccgggacgcgccacctgaggggccatccgctaagaaaccgaagaaggaggcctcgcaaaaacagaaagggggaggccaagggaagaagaagaagaaccaagggaagaagaaggctaagacagggccgcctaatccgaaggcacagaatggaaacaagaagaagaccaacaagaaaccaggcaagagacagcgcatggtcatgaaattggaatctgacaagacgttcccaatcatgttggaagggaagataaacggctacgcttgtgtggtcggagggaagttattcaggccgatgcatgtggaaggcaagatcgacaacgacgttctggccgcgcttaagacgaagaaagcatccaaatacgatcttgagtatgcagatgtgccacagaacatgcgggccgatacattcaaatacacccatgagaaaccccaaggctattacagctggcatcatggagcagtccaatatgaaaatgggcgtttcacggtgccgaaaggagttggggccaagggagacagcggacgacccattctggataaccagggacgggtggtcgctattgtgctgggaggtgtgaatgaaggatctaggacagccctttcagtcgtcatgtggaacgagaagggagttaccgtgaagtatactccggagaactgcgagcaatggtcactagtgaccaccatgtgtctgctcgccaatgtgacgttcccatgtgctcaaccaccaatttgctacgacagaaaaccagcagagactttggccatgctcagcgttaacgttgacaacccgggctacgatgagctgctggaagcagctgttaagtgccccggaaggaaaaggagatccaccgaggagctgtttaaggagtataagctaacgcgcccttacatggccagatgcatcagatgtgcagttgggagctgccatagtccaatagcaatcgaggcagtaaagagcgacgggcacgacggttatgttagacttcagacttcctcgcagtatggcctggattcctccggcaacttaaagggcaggaccatgcggtatgacatgcacgggaccattaaagagataccactacatcaagtgtcactccatacatctcgcccgtgtcacattgtggatgggcacggttatttcctgcttgccaggtgcccggcaggggactccatcaccatggaatttaagaaagattccgtcacacactcctgctcggtgccgtatgaagtgaaatttaatcctgtaggcagagaactctatactcatcccccagaacacggagtagagcaagcgtgccaagtctacgcacatgatgcacagaacagaggagcttatgtcgagatgcacctcccgggctcagaagtggacagcagtttggtttccttgagcggcagttcagtcaccgtgacacctcctgttgggactagcgccctggtggaatgcgagtgtggcggcacaaagatctccgagaccatcaacaagacaaaacagttcagccagtgcacaaagaaggagcagtgcagagcatatcggctgcagaacgataagtgggtgtataattctgacaaactgcccaaagcagcgggagccaccttaaaaggaaaactgcatgtcccattcttgctggcagacggcaaatgcaccgtgcctctagcaccagaacctatgataacctttggtttcagatcagtgtcactgaaactgcaccctaagaatcccacatatctaaccacccgccaacttgctgatgagcctcactacacgcacgagctcatatctgaaccagctgttaggaattttaccgtcaccgaaaaagggtgggagtttgtatggggaaaccacccgccgaaaaggttttgggcacaggaaacagcacccggaaatccacatgggctaccgcacgaggtgataactcattattaccacagataccctatgtccaccatcctgggtttgtcaatttgtgccgccattgcaaccgtttccgttgcagcgtctacctggctgttttgcagatctagagttgcgtgcctaactccttaccggctaacacctaacgctaggataccattttgtctggctgtgctttgctgcgcccgcactgcccgggccgagaccacctgggagtccttggatcacctatggaacaataaccaacagatgttctggattcaattgctgatccctctggccgccttgatcgtagtgactcgcctgctcaggtgcgtgtgctgtgtcgtgccttttttagtcatggccggcgccgcaggcgccggcgcctacgagcacgcgaccacgatgccgagccaagcgggaatctcgtataacactatagtcaacagagcaggctacgcaccactccctatcagcataacaccaacaaagatcaagctgatacctacagtgaacttggagtacgtcacctgccactacaaaacaggaatggattcaccagccatcaaatgctgcggatctcaggaatgcactccaacttacaggcctgatgaacagtgcaaagtcttcacaggggtttacccgttcatgtggggtggtgcatattgcttttgcgacactgagaacacccaagtcagcaaggcctacgtaatgaaatctgacgactgccttgcggatcatgctgaagcatataaagcgcacacagcctcagtgcaggcgttcctcaacatcacagtgggagaacactctattgtgactaccgtgtatgtgaatggagaaactcctgtgaatttcaatggggtcaaattaactgcaggtccgctttccacagcttggacaccctttgatcgcaaaatcgtgcagtatgccggggagatctataattatgattttcctgagtatggggcaggacaaccaggagcatttggagatatacaatccagaacagtctcaagctcagatctgtatgccaataccaacctagtgctgcagagacccaaagcaggagcgatccacgtgccatacactcaggcaccttcgggttttgagcaatggaagaaagataaagctccatcattgaaatttaccgcccctttcggatgcgaaatatatacaaaccccattcgcgccgaaaactgtgctgtagggtcaattccattagcctttgacattcccgacgccttgttcaccagggtgtcagaaacaccgacactttcagcggccgaatgcactcttaacgagtgcgtgtattcttccgactttggtgggatcgccacggtcaagtactcggccagcaagtcaggcaagtgcgcagtccatgtgccatcagggactgctaccctaaaagaagcagcagtcgagctaaccgagcaagggtcggcgactatccatttctcgaccgcaaatatccacccggagttcaggctccaaatatgcacatcatatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttc

SEQ ID NO: 4 - DNA, 9577 bp, synthetic construct
  misc_feature 1..9577: note = Description of Artificial Sequence: Synthetic polynucleotide
  source 1..9577: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgactctagaatagtctttaattaagccaccatggcaggcatgtttcaggcgctgagcgaaggctgcaccccgtatgatattaaccagatgctgaacgtgctgggcgatcatcaggtctcaggccttgagcagcttgagagtataatcaactttgaaaaactgactgaatggaccagttctaatgttatgcctatcctgtctcctctgacaaagggcatcctgggcttcgtgtttaccctgaccgtgccttctgagagaggacttagctgcattagcgaagcggatgcgaccaccccggaaagcgcgaacctgggcgaagaaattctgagccagctgtatctttggccaagggtgacctaccattcccctagttatgcttaccaccaatttgaaagacgagccaaatataaaagacacttccccggctttggccagagcctgctgtttggctaccctgtgtacgtgttcggcgattgcgtgcagggcgattgggatgcgattcgctttcgctattgcgcgccgccgggctatgcgctgctgcgctgcaacgataccaactatagcgctctgctggctgtgggggccctagaaggacccaggaatcaggactggcttggtgtcccaagacaacttgtaactcggatgcaggctattcagaatgccggcctgtgtaccctggtggccatgctggaagagacaatcttctggctgcaagcgtttctgatggcgctgaccgatagcggcccgaaaaccaacattattgtggatagccagtatgtgatgggcattagcaaaccgagctttcaggaatttgtggattgggaaaacgtgagcccggaactgaacagcaccgatcagccgttttggcaagccggaatcctggccagaaatctggtgcctatggtggccacagtgcagggccagaacctgaagtaccagggtcagtcactagtcatctctgcttctatcattgtcttcaacctgctggaactggaaggtgattatcgagatgatggcaacgtgtgggtgcataccccgctgagcccgcgcaccctgaacgcgtgggtgaaagcggtggaagaaaaaaaaggtattccagttcacctagagctggccagtatgaccaacatggagctcatgagcagtattgtgcatcagcaggtcagaacatacggccccgtgttcatgtgtctcggcggactgcttacaatggtggctggtgctgtgtggctgacagtgcgagtgctcgagctgttccgggccgcgcagctggccaacgacgtggtcctccagatcatggagctttgtggtgcagcgtttcgccaggtgtgccataccaccgtgccgtggccgaacgcgagcctgaccccgaaatggaacaacgaaaccacccagccccagatcgccaactgcagcgtgtatgacttttttgtgtggctccattattattctgttcgagacacactttggccaagggtgacctaccatatgaacaaatatgcgtatcatatgctggaaagacgagccaaatataaaagaggaccaggacctggcgctaaatttgtggccgcctggacactgaaagccgctgctggtcctggacctggccagtacatcaaggccaacagcaagttcatcggcatcaccgaactcggacccggaccaggctgatgattcgaacggccgtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 5 - DNA, 11447 bp, Venezuelan equine encephalitis virus; identical to SEQ ID NO: 3
  source 1..11447: mol_type = other DNA

SEQ ID NO: 6 - DNA, 7894 bp, synthetic construct
  misc_feature 1..7894: note = Description of Artificial Sequence: Synthetic polynucleotide
  source 1..7894: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgactatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 7 - DNA, 7893 bp, synthetic construct
  misc_feature 1..7893: note = Description of Artificial Sequence: Synthetic polynucleotide
  source 1..7893: mol_type = other DNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgactatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 8 - DNA, 7927 bp, synthetic construct
  misc_feature 1..7927: note = Description of Artificial Sequence: Synthetic polynucleotide
  source 1..7927: mol_type = other DNA
taatacgactcactataggatgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgactatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaaaaaaaaaaaaaaaaaaaaaaaaaaaaaaaaaaaaatacgtagtttaaac

SEQ ID NO: 9 - DNA, 7926 bp, synthetic construct
  misc_feature 1..7926: note = Description of Artificial Sequence: Synthetic polynucleotide
  source 1..7926: mol_type = other DNA
taatacgactcactataggataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgactatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaaaaaaaaaaaaaaaaaaaaaaaaaaaaaaaaaaaaaaaaaatacgtagtttaaac

SEQ ID NO: 10 - DNA, 36519 bp, synthetic construct
  misc_feature 1..36519: note = Description of Artificial Sequence: Synthetic polynucleotide
  source 1..36519: mol_type = other DNA
ccatcttcaataatatacctcaaactttttgtgcgcgttaatatgcaaatgaggcgtttgaatttggggaggaagggcggtgattggtcgagggatgagcgaccgttaggggcggggcgagtgacgttttgatgacgtggttgcgaggaggagccagtttgcaagttctcgtgggaaaagtgacgtcaaacgaggtgtggtttgaacacggaaatactcaattttcccgcgctctctgacaggaaatgaggtgtttctgggcggatgcaagtgaaaacgggccattttcgcgcgaaaactgaatgaggaagtgaaaatctgagtaatttcgcgtttatggcagggaggagtatttgccgagggccgagtagactttgaccgattacgtgggggtttcgattaccgtgtttttcacctaaatttccgcgtacggtgtcaaagtccggtgtttttacgtaggtgtcagctgatcgccagggtatttaaacctgcgctctccagtcaagaggccactcttgagtgccagcgagaagagttttctcctccgcgccgcgagtcagatctacactttgaaagatgaggcacctgagagacctgcccgatgagaaaatcatcatcgcttccgggaacgagattctggaactggtggtaaatgccatgatgggcgacgaccctccggagccccccaccccatttgagacaccttcgctgcacgatttgtatgatctggaggtggatgtgcccgaggacgatcccaatgaggaggcggtaaatgatttttttagcgatgccgcgctgctagctgccgaggaggcttcgagctctagctcagacagcgactcttcactgcatacccctagacccggcagaggtgagaaaaagatccccgagcttaaaggggaagagatggacttgcgctgctatgaggaatgcttgcccccgagcgatgatgaggacgagcaggcgatccagaacgcagcgagccagggagtgcaagccgccagcgagagctttgcgctggactgcccgcctctgcccggacacggctgtaagtcttgtgaatttcatcgcatgaatactggagataaagctgtgttgtgtgcactttgctatatgagagcttacaaccattgtgtttacagtaagtgtgattaagttgaactttagagggaggcagagagcagggtgactgggcgatgactggtttatttatgtatatatgttctttatataggtcccgtctctgacgcagatgatgagacccccactacaaagtccacttcgtcacccccagaaattggcacatctccacctgagaatattgttagaccagttcctgttagagccactgggaggagagcagctgtggaatgtttggatgacttgctacagggtggggttgaacctttggacttgtgtacccggaaacgccccaggcactaagtgccacacatgtgtgtttacttgaggtgatgtcagtatttatagggtgtggagtgcaataaaaaatgtgttgactttaagtgcgtggtttatgactcaggggtggggactgtgagtatataagcaggtgcagacctgtgtggttagctcagagcggcatggagatttggacggtcttggaagactttcacaagactagacagctgctagagaacgcctcgaacggagtctcttacctgtggagattctgcttcggtggcgacctagctaggctagtctacagggccaaacaggattatagtgaacaatttgaggttattttgagagagtgttctggtctttttgacgctcttaacttgggccatcagtctcactttaaccagaggatttcgagagcccttgattttactactcctggcagaaccactgcagcagtagccttttttgcttttattcttgacaaatggagtcaagaaacccatttcagcagggattaccagctggatttcttagcagtagctttgtggagaacatggaagtgccagcgcctgaatgcaatctccggctacttgccggtacagccgctagacactctgaggatcctgaatctccaggagagtcccagggcacgccaacgtcgccagcagcagcagcaggaggaggatcaagaagagaacccgagagccggcctggaccctccggcggaggaggaggagtagctgacctgtttcctgaactgcgccgggtgctgactaggtcttcgagtggtcgggagagggggattaagcgggagaggcatgatgagactaatcacagaactgaactgactgtgggtctgatgagtcgcaagcgcccagaaacagtgtggtggcatgaggtgcagtcgactggcacagatgaggtgtcggtgatgcatgagaggttttctctagaacaagtcaagacttgttggttagagcctgaggatgattgggaggtagccatcaggaattatgccaagctggctctgaggccagacaagaagtacaagattactaagctgataaatatcagaaatgcctgctacatctcagggaatggggctgaagtggagatctgtctccaggaaagggtggctttcagatgctgcatgatgaatatgtacccgggagtggtgggcatggatggggttacctttatgaacatgaggttcaggggagatgggtataatggcacggtctttatggccaataccaagctgacagtccatggctgctccttctttgggtttaataacacctgcatcgaggcctggggtcaggtcggtgtgaggggctgcagtttttcagccaactggatgggggtcgtgggcaggaccaagagtatgctgtccgtgaagaaatgcttgtttgagaggtgccacctgggggtgatgagcgagggcgaagccagaatccgccactgcgcctctaccgagacgggctgctttgtgctgtgcaagggcaatgctaagatcaagcataatatgatctgtggagcctcggacgagcgcggctaccagatgctgacctgcgccggcgggaacagccatatgctggccaccgtacatgtggcttcccatgctcgcaagccctggcccgagttcgagcacaatgtcatgaccaggtgcaatatgcatctggggtcccgccgaggcatgttcatgccctaccagtgcaacctgaattatgtgaaggtgctgctggagcccgatgccatgtccagagtgagcctgacgggggtgtttgacatgaatgtggaggtgtggaagattctgagatatgatgaatccaagaccaggtgccgagcctgcgagtgcggagggaagcatgccaggttccagcccgtgtgtgtggatgtgacggaggacctgcgacccgatcatttggtgttgccctgcaccgggacggagttcggttccagcggggaagaatctgactagagtgagtagtgttctggggcgggggaggacctgcatgagggccagaataactgaaatctgtgcttttctgtgtgttgcagcagcatgagcggaagcggctcctttgagggaggggtattcagcccttatctgacggggcgtctcccctcctgggcgggagtgcgtcagaatgtgatgggatccacggtggacggccggcccgtgcagcccgcgaactcttcaaccctgacctatgcaaccctgagctcttcgtcgttggacgcagctgccgccgcagctgctgcatctgccgccagcgccgtgcgcggaatggccatgggcgccggctactacggcactctggtggccaactcgagttccaccaataatcccgccagcctgaacgaggagaagctgttgctgctgatggcccagctcgaggccttgacccagcgcctgggcgagctgacccagcaggtggctcagctgcaggagcagacgcgggccgcggttgccacggtgaaatccaaataaaaaatgaatcaataaataaacggagacggttgttgattttaacacagagtctgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcatggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcctttgtgcccgcccaggttttccatgcactcatccatgatgatggcgatgggcccgtgggcggcggcctgggcaaagacgtttcgggggtcggacacatcatagttgtggtcctgggtgaggtcatcataggccattttaatgaatttggggcggagggtgccggactgggggacaaaggtaccctcgatcccgggggcgtagttcccctcacagatctgcatctcccaggctttgagctcggagggggggatcatgtccacctgcggggcgataaagaacacggtttccggggcgggggagatgagctgggccgaaagcaagttccggagcagctgggacttgccgcagccggtggggccgtagatgaccccgatgaccggctgcaggtggtagttgagggagagacagctgccgtcctcccggaggaggggggccacctcgttcatcatctcgcgcacgtgcatgttctcgcgcaccagttccgccaggaggcgctctccccccagggataggagctcctggagcgaggcgaagtttttcagcggcttgagtccgtcggccatgggcattttggagagggtttgttgcaagagttccaggcggtcccagagctcggtgatgtgctctacggcatctcgatccagcagacctcctcgtttcgcgggttgggacggctgcgggagtagggcaccagacgatgggcgtccagcgcagccagggtccggtccttccagggtcgcagcgtccgcgtcagggtggtctccgtcacggtgaaggggtgcgcgccgggctgggcgcttgcgagggtgcgcttcaggctcatccggctggtcgaaaaccgctcccgatcggcgccctgcgcgtcggccaggtagcaattgaccatgagttcgtagttgagcgcctcggccgcgtggcctttggcgcggagcttacctttggaagtctgcccgcaggcgggacagaggagggacttgagggcgtagagcttgggggcgaggaagacggactcgggggcgtaggcgtccgcgccgcagtgggcgcagacggtctcgcactccacgagccaggtgaggtcgggctggtcggggtcaaaaaccagtttcccgccgttctttttgatgcgtttcttacctttggtctccatgagctcgtgtccccgctgggtgacaaagaggctgtccgtgtccccgtagaccgactttatgggccggtcctcgagcggtgtgccgcggtcctcctcgtagaggaaccccgcccactccgagacgaaagcccgggtccaggccagcacgaaggaggccacgtgggacgggtagcggtcgttgtccaccagcgggtccaccttttccagggtatgcaaacacatgtccccctcgtccacatccaggaaggtgattggcttgtaagtgtaggccacgtgaccgggggtcccggccgggggggtataaaagggtgcgggtccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccggcggagatgcctttcaagagcccctcgtccatctggtcagaaaagacgatctttttgttgtcgagcttggtggcgaaggagccgtagagggcgttggagaggagcttggcgatggagcgcatggtctggtttttttccttgtcggcgcgctccttggcggcgatgttgagctgcacgtactcgcgcgccacgcacttccattcggggaagacggtggtcagctcgtcgggcacgattctgacctgccagccccgattatgcagggtgatgaggtccacactggtggccacctcgccgcgcaggggctcattagtccagcagaggcgtccgcccttgcgcgagcagaaggggggcagggggtccagcatgacctcgtcgggggggtcggcatcgatggtgaagatgccgggcaggaggtcggggtcaaagtagctgatggaagtggccagatcgtccagggcagcttgccattcgcgcacggccagcgcgcgctcgtagggactgaggggcgtgccccagggcatgggatgggtaagcgcggaggcgtacatgccgcagatgtcgtagacgtagaggggctcctcgaggatgccgatgtaggtggggtagcagcgccccccgcggatgctggcgcgcacgtagtcatacagctcgtgcgagggggcgaggagccccgggcccaggttggtgcgactgggcttttcggcgcggtagacgatctggcggaaaatggcatgcgagttggaggagatggtgggcctttggaagatgttgaagtgggcgtggggcagtccgaccgagtcgcggatgaagtgggcgtaggagtcttgcagcttggcgacgagctcggcggtgactaggacgtccagagcgcagtagtcgagggtctcctggatgatgtcatacttgagctgtcccttttgtttccacagctcgcggttgagaaggaactcttcgcggtccttccagtactcttcgagggggaacccgtcctgatctgcacggtaagagcctagcatgtagaactggttgacggccttgtaggcgcagcagcccttctccacggggagggcgtaggcctgggcggccttgcgcagggaggtgtgcgtgagggcgaaagtgtccctgaccatgaccttgaggaactggtgcttgaagtcgatatcgtcgcagcccccctgctcccagagctggaagtccgtgcgcttcttgtaggcggggttgggcaaagcgaaagtaacatcgttgaagaggatcttgcccgcgcggggcataaagttgcgagtgatgcggaaaggttggggcacctcggcccggttgttgatgacctgggcggcgagcacgatctcgtcgaagccgttgatgttgtggcccacgatgtagagttccacgaatcgcggacggcccttgacgtggggcagtttcttgagctcctcgtaggtgagctcgtcggggtcgctgagcccgtgctgctcgagcgcccagtcggcgagatgggggttggcgcggaggaaggaagtccagagatccacggccagggcggtttgcagacggtcccggtactgacggaactgctgcccgacggccattttttcgggggtgacgcagtagaaggtgcgggggtccccgtgccagcgatcccatttgagctggagggcgagatcgagggcgagctcgacgagccggtcgtccccggagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccattggtggcgacgtcgatggcttgcagggtcccgtgcccctggggtgtgaccaccgtcccccgtttcttcttgggcggctggggcgacgggggcggtgcctcttccatggttagaagcggcggcgaggacgcgcgccgggcggcaggggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcttgaaggtctccgcggccggcgcgctccacggtggccgcgaggtcgttggagatgcggcccatgagctgcgagaaggcgttcatgcccgcctcgttccagacgcggctgtagaccacgacgccctcgggatcgcgggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaaacgttccatggcctcgtaaaagtccacggcgaagttgaaaaactgggagttgcgcgccgagacggtcaactcctcctccagaagacggatgagctcggcgatggtggcgcgcacctcgcgctcgaaggcccccgggagttcctccacttcctcttcttcctcctccactaacatctcttctacttcctcctcaggcggcagtggtggcgggggagggggcctgcgtcgccggcggcgcacgggcagacggtcgatgaagcgctcgatggtctcgccgcgccggcgtcgcatggtctcggtgacggcgcgcccgtcctcgcggggccgcagcgtgaagacgccgccgcgcatctccaggtggccgggggggtccccgttgggcagggagagggcgctgacgatgcatcttatcaattgccccgtagggactccgcgcaaggacctgagcgtctcgagatccacgggatctgaaaaccgctgaacgaaggcttcgagccagtcgcagtcgcaaggtaggctgagcacggtttcttctggcgggtcatgttggttgggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccaggtccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcttgctggatctgggtgagggtggtctggaagtcatcaaagtcgacgaagcggtggtaggctccggtgttgatggtgtaggagcagttggccatgacggaccagttgacggtctggtggcccggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtattggcactcccgtctcgacccaagcctgcaccaaccctccaggatacggaggcgggtcgttttgcaacttttttttggaggccggatgagactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagaccccatagccagccgacttctccagttacggagcgagcccctcttttgttttgtttgtttttgccagatgcatcccgtactgcggcagatgcgcccccaccaccctccaccgcaacaacagccccctccacagccggcgcttctgcccccgccccagcagcaacttccagccacgaccgccgcggccgccgtgagcggggctggacagagttatgatcaccagctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaagcgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagtttgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgtgcggtgagcgccagcaggcggcgcgagctgagcgaccaggagctgatgcatagtctgcagcgggccctgaccggggccgggaccgagggggagagctactttgacatgggcgcggacctgcactggcagcccagccgccgggccttggaggcggcggcaggaccctacgtagaagaggtggacgatgaggtggacgaggagggcgagtacctggaagactgatggcgcgaccgtatttttgctagatgcaacaacaacagccacctcctgatcccgcgatgcgggcggcgctgcagagccagccgtccggcattaactcctcggacgattggacccaggccatgcaacgcatcatggcgctgacgacccgcaaccccgaagcctttagacagcagccccaggccaaccggctctcggccatcctggaggccgtggtgccctcgcgctccaaccccacgcacgagaaggtcctggccatcgtgaacgcgctggtggagaacaaggccatccgcggcgacgaggccggcctggtgtacaacgcgctgctggagcgcgtggcccgctacaacagcaccaacgtgcagaccaacctggaccgcatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggctttcaagaacttgcagggcctgtggggcgtgcaggccccggtcggggaccgcgcgacggtgtcgagcctgctgacgccgaactcgcgcctgctgctgctgctggtggcccccttcacggacagcggcagcatcaaccgcaactcgtacctgggctacctgattaacctgtaccgcgaggccatcggccaggcgcacgtggacgagcagacctaccaggagatcacccacgtgagccgcgccctgggccaggacgacccgggcaacctggaagccaccctgaactttttgctgaccaaccggtcgcagaagatcccgccccagtacgcgctcagcaccgaggaggagcgcatcctgcgttacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctgaatccccactggctcccgccgccggggttctacacgggcgagtacgacatgcccgaccccaatgacgggttcctgtgggacgatgtggacagcagcgtgttctccccccgaccgggtgctaacgagcgccccttgtggaagaaggaaggcagcgaccgacgcccgtcctcggcgctgtccggccgcgagggtgctgccgcggcggtgcccgaggccgccagtcctttcccgagcttgcccttctcgctgaacagtatccgcagcagcgagctgggcaggatcacgcgcccgcgcttgctgggcgaagaggagtacttgaatgactcgctgttgagacccgagcgggagaagaacttccccaataacgggatagaaagcctggtggacaagatgagccgctggaagacgtatgcgcaggagcacagggacgatccccgggcgtcgcagggggccacgagccggggcagcgccgcccgtaaacgccggtggcacgacaggcagcggggacagatgtgggacgatgaggactccgccgacgacagcagcgtgttggacttgggtgggagtggtaacccgttcgctcacctgcgcccccgtatcgggcgcatgatgtaagagaaaccgaaaataaatgatactcaccaaggccatggcgaccagcgtgcgttcgtttcttctctgttgttgttgtatctagtatgatgaggcgtgcgtacccggagggtcctcctccctcgtacgagagcgtgatgcagcaggcgatggcggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggccagctgaaaaccatcatgcacaccaacatgcccaacgtgaacgagttcatgtacagcaacaagttcaaggcgcgggtgatggtctcccgcaagacccccaatggggtgacagtgacagaggattatgatggtagtcaggatgagctgaagtatgaatgggtggaatttgagctgcccgaaggcaacttctcggtgaccatgaccatcgacctgatgaacaacgccatcatcgacaattacttggcggtggggcggcagaacggggtgctggagagcgacatcggcgtgaagttcgacactaggaacttcaggctgggctgggaccccgtgaccgagctggtcatgcccggggtgtacaccaacgaggctttccatcccgatattgtcttgctgcccggctgcggggtggacttcaccgagagccgcctcagcaacctgctgggcattcgcaagaggcagcccttccaggaaggcttccagatcatgtacgaggatctggaggggggcaacatccccgcgctcctggatgtcgacgcctatgagaaaagcaaggaggatgcagcagctgaagcaactgcagccgtagctaccgcctctaccgaggtcaggggcgataattttgcaagcgccgcagcagtggcagcggccgaggcggctgaaaccgaaagtaagatagtcattcagccggtggagaaggatagcaagaacaggagctacaacgtactaccggacaagataaacaccgcctaccgcagctggtacctagcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgcttacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcggtcgcgcaccaccgtcgacgacgtgatcgaccaggtggtggccgacgcgcgcaactacacccccgccgccgcgcccgtctccaccgtggacgccgtcatcgacagcgtggtggccgacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcttcaggcgccagcgccggcaggacccggagacgcgcggccacggcggcggcagcggccatcgccagcatgtcccgcccgcggcgagggaacgtgtactgggtgcgcgacgccgccaccggtgtgcgcgtgcccgtgcgcacccgcccccctcgcacttgaagatgttcacttcgcgatgttgatgtgtcccagcggcgaggaggatgtccaagcgcaaattcaaggaagagatgctccaggtcatcgcgcctgagatctacggccctgcggtggtgaaggaggaaagaaagccccgcaaaatcaagcgggtcaaaaaggacaaaaaggaagaagaaagtgatgtggacggattggtggagtttgtgcgcgagttcgccccccggcggcgcgtgcagtggcgcgggcggaaggtgcaaccggtgctgagacccggcaccaccgtggtcttcacgcccggcgagcgctccggcaccgcttccaagcgctcctacgacgaggtgtacggggatgatgatattctggagcaggcggccgagcgcctgggcgagtttgcttacggcaagcgcagccgttccgcaccgaaggaagaggcggtgtccatcccgctggaccacggcaaccccacgccgagcctcaagcccgtgaccttgcagcaggtgctgccgaccgcggcgccgcgccgggggttcaagcgcgagggcgaggatctgtaccccaccatgcagctgatggtgcccaagcgccagaagctggaagacgtgctggagaccatgaaggtggacccggacgtgcagcccgaggtcaaggtgcggcccatcaagcaggtggccccgggcctgggcgtgcagaccgtggacatcaagattcccacggagcccatggaaacgcagaccgagcccatgatcaagcccagcaccagcaccatggaggtgcagacggatccctggatgccatcggctcctagtcgaagaccccggcgcaagtacggcgcggccagcctgctgatgcccaactacgcgctgcatccttccatcatccccacgccgggctaccgcggcacgcgcttctaccgcggtcataccagcagccgccgccgcaagaccaccactcgccgccgccgtcgccgcaccgccgctgcaaccacccctgccgccctggtgcggagagtgtaccgccgcggccgcgcacctctgaccctgccgcgcgcgcgctaccacccgagcatcgccatttaaactttcgcctgctttgcagatcaatggccctcacatgccgccttcgcgttcccattacgggctaccgaggaagaaaaccgcgccgtagaaggctggcggggaacgggatgcgtcgccaccaccaccggcggcggcgcgccatcagcaagcggttggggggaggcttcctgcccgcgctgatccccatcatcgccgcggcgatcggggcgatccccggcattgcttccgtggcggtgcaggcctctcagcgccactgagacacacttggaaacatcttgtaataaaccaatggactctgacgctcctggtcctgtgatgtgttttcgtagacagatggaagacatcaatttttcgtccctggctccgcgacacggcacgcggccgttcatgggcacctggagcgacatcggcaccagccaactgaacgggggcgccttcaattggagcagtctctggagcgggcttaagaatttcgggtccacgcttaaaacctatggcagcaaggcgtggaacagcaccacagggcaggcgctgagggataagctgaaagagcagaacttccagcagaaggtggtcgatgggctcgcctcgggcatcaacggggtggtggacctggccaaccaggccgtgcagcggcagatcaacagccgcctggacccggtgccgcccgccggctccgtggagatgccgcaggtggaggaggagctgcctcccctggacaagcggggcgagaagcgaccccgccccgatgcggaggagacgctgctgacgcacacggacgagccgcccccgtacgaggaggcggtgaaactgggtctgcccaccacgcggcccatcgcgcccctggccaccggggtgctgaaacccgaaaagcccgcgaccctggacttgcctcctccccagccttcccgcccctctacagtggctaagcccctgccgccggtggccgtggcccgcgcgcgacccgggggcaccgcccgccctcatgcgaactggcagagcactctgaacagcatcgtgggtctgggagtgcagagtgtgaagcgccgccgctgctattaaacctaccgtagcgcttaacttgcttgtctgtgtgtgtatgtattatgtcgccgccgccgctgtccaccagaaggaggagtgaagaggcgcgtcgccgagttgcaagatggccaccccatcgatgctgccccagtgggcgtacatgcacatcgccggacaggacgcttcggagtacctgagtccgggtctggtgcagtttgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccctagcttcaaaccctactccggcaccgcctacaacagtctggcccccaagggagcacccaacacttgtcagtggacatataaagccgatggtgaaactgccacagaaaaaacctatacatatggaaatgcacccgtgcagggcattaacatcacaaaagatggtattcaacttggaactgacaccgatgatcagccaatctacgcagataaaacctatcagcctgaacctcaagtgggtgatgctgaatggcatgacatcactggtactgatgaaaagtatggaggcagagctcttaagcctgataccaaaatgaagccttgttatggttcttttgccaagcctactaataaagaaggaggtcaggcaaatgtgaaaacaggaacaggcactactaaagaatatgacatagacatggctttctttgacaacagaagtgcggctgctgctggcctagctccagaaattgttttgtatactgaaaatgtggatttggaaactccagatacccatattgtatacaaagcaggcacagatgacagcagctcttctattaatttgggtcagcaagccatgcccaacagacctaactacattggtttcagagacaactttatcgggctcatgtactacaacagcactggcaatatgggggtgctggccggtcaggcttctcagctgaatgctgtggttgacttgcaagacagaaacaccgagctgtcctaccagctcttgcttgactctctgggtgacagaacccggtatttcagtatgtggaatcaggcggtggacagctatgatcctgatgtgcgcattattgaaaatcatggtgtggaggatgaacttcccaactattgtttccctctggatgctgttggcagaacagatacttatcagggaattaaggctaatggaactgatcaaaccacatggaccaaagatgacagtgtcaatgatgctaatgagataggcaagggtaatccattcgccatggaaatcaacatccaagccaacctgtggaggaacttcctctacgccaacgtggccctgtacctgcccgactcttacaagtacacgccggccaatgttaccctgcccaccaacaccaacacctacgattacatgaacggccgggtggtggcgccctcgctggtggactcctacatcaacatcggggcgcgctggtcgctggatcccatggacaacgtgaaccccttcaaccaccaccgcaatgcggggctgcgctaccgctccatgctcctgggcaacgggcgctacgtgcccttccacatccaggtgccccagaaatttttcgccatcaagagcctcctgctcctgcccgggtcctacacctacgagtggaacttccgcaaggacgtcaacatgatcctgcagagctccctcggcaacgacctgcgcacggacggggcctccatctccttcaccagcatcaacctctacgccaccttcttccccatggcgcacaacacggcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggctggtccttcacgcgtctcaagaccaaggagacgccctcgctgggctccgggttcgacccctacttcgtctactcgggctccatcccctacctcgacggcaccttctacctcaaccacaccttcaagaaggtctccatcaccttcgactcctccgtcagctggcccggcaacgaccggctcctgacgcccaacgagttcgaaatcaagcgcaccgtcgacggcgagggctacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtggtggacgaggtcaactacaaggactaccaggccgtcaccctggcctaccagcacaacaactcgggcttcgtcggctacctcgcgcccaccatgcgccagggccagccctaccccgccaactacccctacccgctcatcggcaagagcgccgtcaccagcgtcacccagaaaaagttcctctgcgacagggtcatgtggcgcatccccttctccagcaacttcatgtccatgggcgcgctcaccgacctcggccagaacatgctctatgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacccccttctcggccggtaacgccaccacctaagctcttgcttcttgcaagccatggccgcgggctccggcgagcaggagctcagggccatcatccgcgacctgggctgcgggccctacttcctgggcaccttcgataagcgcttcccgggattcatggccccgcacaagctggcctgcgccatcgtcaacacggccggccgcgagaccgggggcgagcactggctggccttcgcctggaacccgcgctcgaacacctgctacctcttcgaccccttcgggttctcggacgagcgcctcaagcagatctaccagttcgagtacgagggcctgctgcgccgcagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaactcccactccgcctactttcgctcccaccgcgcgcgcatcgagaaggccaccgccttcgaccgcatgaatcaagacatgtaaaccgtgtgtgtatgttaaatgtctttaataaacagcactttcatgttacacatgcatctgagatgatttatttagaaatcgaaagggttctgccgggtctcggcatggcccgcgggcagggacacgttgcggaactggtacttggccagccacttgaactcggggatcagcagtttgggcagcggggtgtcggggaaggagtcggtccacagcttccgcgtcagttgcagggcgcccagcaggtcgggcgcggagatcttgaaatcgcagttgggacccgcgttctgcgcgcgggagttgcggtacacggggttgcagcactggaacaccatcagggccgggtgcttcacgctcgccagcaccgtcgcgtcggtgatgctctccacgtcgaggtcctcggcgttggccatcccgaagggggtcatcttgcaggtctgccttcccatggtgggcacgcacccgggcttgtggttgcaatcgcagtgcagggggatcagcatcatctgggcctggtcggcgttcatccccgggtacatggccttcatgaaagcctccaattgcctgaacgcctgctgggccttggctccctcggtgaagaagaccccgcaggacttgctagagaactggttggtggcgcacccggcgtcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcatgtgctccttctggatcatggtggtcccgtgcaggcaccgcagcttgccctcggcctcggtgcacccgtgcagccacagcgcgcacccggtgcactcccagttcttgtgggcgatctgggaatgcgcgtgcacgaagccctgcaggaagcggcccatcatggtggtcagggtcttgttgctagtgaaggtcagcggaatgccgcggtgctcctcgttgatgtacaggtggcagatgcggcggtacacctcgccctgctcgggcatcagctggaagttggctttcaggtcggtctccacgcggtagcggtccatcagcatagtcatgatttccatacccttctcccaggccgagacgatgggcaggctcatagggttcttcaccatcatcttagcgctagcagccgcggccagggggtcgctctcgtccagggtctcaaagctccgcttgccgtccttctcggtgatccgcaccggggggtagctgaagcccacggccgccagctcctcctcggcctgtctttcgtcctcgctgtcctggctgacgtcctgcaggaccacatgcttggtcttgcggggtttcttcttgggcggcagcggcggcggagatgttggagatggcgagggggagcgcgagttctcgctcaccactactatctcttcctcttcttggtccgaggccacgcggcggtaggtatgtctcttcgggggcagaggcggaggcgacgggctctcgccgccgcgacttggcggatggctggcagagccccttccgcgttcgggggtgcgctcccggcggcgctctgactgacttcctccgcggccggccattgtgttctcctagggaggaacaacaagcatggagactcagccatcgccaacctcgccatctgcccccaccgccgacgagaagcagcagcagcagaatgaaagcttaaccgccccgccgcccagccccgccacctccgacgcggccgtcccagacatgcaagagatggaggaatccatcgagattgacctgggctatgtgacgcccgcggagcacgaggaggagctggcagtgcgcttttcacaagaagagatacaccaagaacagccagagcaggaagcagagaatgagcagagtcaggctgggctcgagcatgacggcgactacctccacctgagcgggggggaggacgcgctcatcaagcatctggcccggcaggccaccatcgtcaaggatgcgctgctcgaccgcaccgaggtgcccctcagcgtggaggagctcagccgcgcctacgagttgaacctcttctcgccgcgcgtgccccccaagcgccagcccaatggcacctgcgagcccaacccgcgcctcaacttctacccggtcttcgcggtgcccgaggccctggccacctaccacatctttttcaagaaccaaaagatccccgtctcctgccgcgccaaccgcacccgcgccgacgcccttttcaacctgggtcccggcgcccgcctacctgatatcgcctccttggaagaggttcccaagatcttcgagggtctgggcagcgacgagactcgggccgcgaacgctctgcaaggagaaggaggagagcatgagcaccacagcgccctggtcgagttggaaggcgacaacgcgcggctggcggtgctcaaacgcacggtcgagctgacccatttcgcctacccggctctgaacctgccccccaaagtcatgagcgcggtcatggaccaggtgctcatcaagcgcgcgtcgcccatctccgaggacgagggcatgcaagactccgaggagggcaagcccgtggtcagcgacgagcagctggcccggtggctgggtcctaatgctagtccccagagtttggaagagcggcgcaaactcatgatggccgtggtcctggtgaccgtggagctggagtgcctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcttgcacgagaaccgcctggggcagaacgtgctgcacaccaccctgcgcggggaggcccggcgcgactacatccgcgactgcgtctacctctacctctgccacacctggcagacgggcatgggcgtgtggcagcagtgtctggaggagcagaacctgaaagagctctgcaagctcctgcagaagaacctcaagggtctgtggaccgggttcgacgagcgcaccaccgcctcggacctggccgacctcattttccccgagcgcctcaggctgacgctgcgcaacggcctgcccgactttatgagccaaagcatgttgcaaaactttcgctctttcatcctcgaacgctccggaatcctgcccgccacctgctccgcgctgccctcggacttcgtgccgctgaccttccgcgagtgccccccgccgctgtggagccactgctacctgctgcgcctggccaactacctggcctaccactcggacgtgatcgaggacgtcagcggcgagggcctgctcgagtgccactgccgctgcaacctctgcacgccgcaccgctccctggcctgcaacccccagctgctgagcgagacccagatcatcggcaccttcgagttgcaagggcccagcgaaggcgagggttcagccgccaaggggggtctgaaactcaccccggggctgtggacctcggcctacttgcgcaagttcgtgcccgaggactaccatcccttcgagatcaggttctacgaggaccaatcccatccgcccaaggccgagctgtcggcctgcgtcatcacccagggggcgatcctggcccaattgcaagccatccagaaatcccgccaagaattcttgctgaaaaagggccgcggggtctacctcgacccccagaccggtgaggagctcaaccccggcttcccccaggatgccccgaggaaacaagaagctgaaagtggagctgccgcccgtggaggatttggaggaagactgggagaacagcagtcaggcagaggaggaggagatggaggaagactgggacagcactcaggcagaggaggacagcctgcaagacagtctggaggaagacgaggaggaggcagaggaggaggtggaagaagcagccgccgccagaccgtcgtcctcggcgggggagaaagcaagcagcacggataccatctccgctccgggtcggggtcccgctcgaccacacagtagatgggacgagaccggacgattcccgaaccccaccacccagaccggtaagaaggagcggcagggatacaagtcctggcgggggcacaaaaacgccatcgtctcctgcttgcaggcctgcgggggcaacatctccttcacccggcgctacctgctcttccaccgcggggtgaactttccccgcaacatcttgcattactaccgtcacctccacagcccctactacttccaagaagaggcagcagcagcagaaaaagaccagcagaaaaccagcagctagaaaatccacagcggcggcagcaggtggactgaggatcgcggcgaacgagccggcgcaaacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccagtcgcagaaaaaggcgggaattacgtcacctgtgcccttcgccctagccgcctccacccatcatcatgagcaaagagattcccacgccttacatgtggagctaccagccccagatgggcctggccgccggtgccgcccaggactactccacccgcatgaattggctcagcgccgggcccgcgatgatctcacgggtgaatgacatccgcgcccaccgaaaccagatactcctagaacagtcagcgctcaccgccacgccccgcaatcacctcaatccgcgtaattggcccgccgccctggtgtaccaggaaattccccagcccacgaccgtactacttccgcgagacgcccaggccgaagtccagctgactaactcaggtgtccagctggcgggcggcgccaccctgtgtcgtcaccgccccgctcagggtataaagcggctggtgatccggggcagaggcacacagctcaacgacgaggtggtgagctcttcgctgggtctgcgacctgacggagtcttccaactcgccggatcggggagatcttccttcacgcctcgtcaggccgtcctgactttggagagttcgtcctcgcagccccgctcgggtggcatcggcactctccagttcgtggaggagttcactccctcggtctacttcaaccccttctccggctcccccggccactacccggacgagttcatcccgaacttcgacgccatcagcgagtcggtggacggctacgattgaatgtcccatggtggcgcagctgacctagctcggcttcgacacctggaccactgccgccgcttccgctgcttcgctcgggatctcgccgagtttgcctactttgagctgcccgaggagcaccctcagggcccggcccacggagtgcggatcgtcgtcgaagggggcctcgactcccacctgcttcggatcttcagccagcgtccgatcctggtcgagcgcgagcaaggacagacccttctgactctgtactgcatctgcaaccaccccggcctgcatgaaagtctttgttgtctgctgtgtactgagtataataaaagctgagatcagcgactactccggacttccgtgtgttcctgaatccatcaaccagtctttgttcttcaccgggaacgagaccgagctccagctccagtgtaagccccacaagaagtacctcacctggctgttccagggctccccgatcgccgttgtcaaccactgcgacaacgacggagtcctgctgagcggccctgccaaccttactttttccacccgcagaagcaagctccagctcttccaacccttcctccccgggacctatcagtgcgtctcgggaccctgccatcacaccttccacctgatcccgaataccacagcgtcgctccccgctactaacaaccaaactaacctccaccaacgccaccgtcgcgacctttctgaatctaatactaccacccacaccggaggtgagctccgaggtcaaccaacctctgggatttactacggcccctgggaggtggttgggttaatagcgctaggcctagttgcgggtgggcttttggttctctgctacctatacctcccttgctgttcgtacttagtggtgctgtgttgctggtttaagaaatggggaagatcaccctagtgagctgcggtgcgctggtggcggtgttgctttcgattgtgggactgggcggtgcggctgtagtgaaggagaaggccgatccctgcttgcatttcaatcccaacaaatgccagctgagttttcagcccgatggcaatcggtgcgcggtactgatcaagtgcggatgggaatgcgagaacgtgagaatcgagtacaataacaagactcggaacaatactctcgcgtccgtgtggcagcccggggaccccgagtggtacaccgtctctgtccccggtgctgacggctccccgcgcaccgtgaataatactttcatttttgcgcacatgtgcgacacggtcatgtggatgagcaagcagtacgatatgtggccccccacgaaggagaacatcgtggtcttctccatcgcttacagcctgtgcacggcgctaatcaccgctatcgtgtgcctgagcattcacatgctcatcgctattcgccccagaaataatgccgaaaaagaaaaacagccataacgttttttttcacacctttttcagaccatggcctctgttaaatttttgcttttatttgccagtctcattgccgtcattcatggaatgagtaatgagaaaattactatttacactggcactaatcacacattgaaaggtccagaaaaagccacagaagtttcatggtattgttattttaatgaatcagatgtatctactgaactctgtggaaacaataacaaaaaaaatgagagcattactctcatcaagtttcaatgtggatctgacttaaccctaattaacatcactagagactatgtaggtatgtattatggaactacagcaggcatttcggacatggaattttatcaagtttctgtgtctgaacccaccacgcctagaatgaccacaaccacaaaaactacacctgttaccactatgcagctcactaccaataacatttttgccatgcgtcaaatggtcaacaatagcactcaacccaccccacccagtgaggaaattcccaaatccatgattggcattattgttgctgtagtggtgtgcatgttgatcatcgccttgtgcatggtgtactatgccttctgctacagaaagcacagactgaacgacaagctggaacacttactaagtgttgaattttaattttttagaaccatgaagatcctaggccttttaattttttctatcattacctctgctctatgcaattctgacaatgaggacgttactgtcgttgtcggatcaaattatacactgaaaggtccagcgaagggtatgctttcgtggtattgctattttggatctgacactacagaaactgaattatgcaatcttaagaatggcaaaattcaaaattctaaaattaacaattatatatgcaatggtactgatctgatactcctcaatatcacgaaatcatatgctggcagttacacctgccctggagatgatgctgacagtatgattttttacaaagtaactgttgttgatcccactactccacctccacccaccacaactactcacaccacacacacagatcaaaccgcagcagaggaggcagcaaagttagccttgcaggtccaagacagttcatttgttggcattacccctacacctgatcagcggtgtccggggctgctagtcagcggcattgtcggtgtgctttcgggattagcagtcataatcatctgcatgttcatttttgcttgctgctatagaaggctttaccgacaaaaatcagacccactgctgaacctctatgtttaattttttccagagtcatgaaggcagttagcgctctagttttttgttctttgattggcattgttttttgcaatcctattcctaaagttagctttattaaagatgtgaatgttactgaggggggcaatgtgacactggtaggtgtagagggtgctgaaaacaccacctggacaaaataccacctcaatgggtggaaagatatttgcaattggagtgtattagtttatacatgtgagggagttaatcttaccattgtcaatgccacctcagctcaaaatggtagaattcaaggacaaagtgtcagtgtatctaatgggtattttacccaacatacttttatctatgacgttaaagtcataccactgcctacgcctagcccacctagcactaccacacagacaacccacactacacagacaaccacatacagtacattaaatcagcctaccaccactacagcagcagaggttgccagctcgtctggggtccgagtggcatttttgatgttggccccatctagcagtcccactgctagtaccaatgagcagactactgaatttttgtccactgtcgagagccacaccacagctacctccagtgccttctctagcaccgccaatctctcctcgctttcctctacaccaatcagtcccgctactactcctagccccgctcctcttcccactcccctgaagcaaacagacggcggcatgcaatggcagatcaccctgctcattgtgatcgggttggtcatcctggccgtgttgctctactacatcttctgccgccgcattcccaacgcgcaccgcaagccggtctacaagcccatcattgtcgggcagccggagccgcttcaggtggaagggggtctaaggaatcttctcttctcttttacagtatggtgattgaactatgattcctagacaattcttgatcactattcttatctgcctcctccaagtctgtgccaccctcgctctggtggccaacgccagtccagactgtattgggcccttcgcctcctacgtgctctttgccttcaccacctgcatctgctgctgtagcatagtctgcctgcttatcaccttcttccagttcattgactggatctttgtgcgcatcgcctacctgcgccaccacccccagtaccgcgaccagcgagtggcgcggctgctcaggctcctctgataagcatgcgggctctgctacttctcgcgcttctgctgttagtgctcccccgtcccgtcgacccccggtcccccacccagtcccccgaggaggtccgcaaatgcaaattccaagaaccctggaaattcctcaaatgctaccgccaaaaatcagacatgcatcccagctggatcatgatcattgggatcgtgaacattctggcctgcaccctcatctcctttgtgatttacccctgctttgactttggttggaactcgccagaggcgctctatctcccgcctgaacctgacacaccaccacagcaacctcaggcacacgcactaccaccactacagcctaggccacaatacatgcccatattagactatgaggccgagccacagcgacccatgctccccgctattagttacttcaatctaaccggcggagatgactgacccactggccaacaacaacgtcaacgaccttctcctggacatggacggccgcgcctcggagcagcgactcgcccaacttcgcattcgccagcagcaggagagagccgtcaaggagctgcaggatgcggtggccatccaccagtgcaagagaggcatcttctgcctggtgaaacaggccaagatctcctacgaggtcactccaaacgaccatcgcctctcctacgagctcctgcagcagcgccagaagttcacctgcctggtcggagtcaaccccatcgtcatcacccagcagtctggcgataccaaggggtgcatccactgctcctgcgactcccccgactgcgtccacactctgatcaagaccctctgcggcctccgcgacctcctccccatgaactaatcacccccttatccagtgaaataaagatcatattgatgatgattttacagaaataaaaaataatcatttgatttgaaataaagatacaatcatattgatgatttgagtttaacaaaaaaataaagaatcacttacttgaaatctgataccaggtctctgtccatgttttctgccaacaccacttcactcccctcttcccagctctggtactgcaggccccggcgggctgcaaacttcctccacacgctgaaggggatgtcaaattcctcctgtccctcaatcttcattttatcttctatcagatgtccaaaaagcgcgtccgggtggatgatgacttcgaccccgtctacccctacgatgcagacaacgcaccgaccgtgcccttcatcaacccccccttcgtctcttcagatggattccaagagaagcccctgggggtgttgtccctgcgactggccgaccccgtcaccaccaagaacggggaaatcaccctcaagctgggagagggggtggacctcgattcctcgggaaaactcatctccaacacggccaccaaggccgccgcccctctcagtttttccaacaacaccatttcccttaacatggatcaccccttttacactaaagatggaaaattatccttacaagtttctccaccattaaatatactgagaacaagcattctaaacacactagctttaggttttggatcaggtttaggactccgtggctctgccttggcagtacagttagtctctccacttacatttgatactgatggaaacataaagcttaccttagacagaggtttgcatgttacaacaggagatgcaattgaaagcaacataagctgggctaaaggtttaaaatttgaagatggagccatagcaaccaacattggaaatgggttagagtttggaagcagtagtacagaaacaggtgttgatgatgcttacccaatccaagttaaacttggatctggccttagctttgacagtacaggagccataatggctggtaacaaagaagacgataaactcactttgtggacaacacctgatccatcaccaaactgtcaaatactcgcagaaaatgatgcaaaactaacactttgcttgactaaatgtggtagtcaaatactggccactgtgtcagtcttagttgtaggaagtggaaacctaaaccccattactggcaccgtaagcagtgctcaggtgtttctacgttttgatgcaaacggtgttcttttaacagaacattctacactaaaaaaatactgggggtataggcagggagatagcatagatggcactccatataccaatgctgtaggattcatgcccaatttaaaagcttatccaaagtcacaaagttctactactaaaaataatatagtagggcaagtatacatgaatggagatgtttcaaaacctatgcttctcactataaccctcaatggtactgatgacagcaacagtacatattcaatgtcattttcatacacctggactaatggaagctatgttggagcaacatttggggctaactcttataccttctcatacatcgcccaagaatgaacactgtatcccaccctgcatgccaacccttcccaccccactctgtggaacaaactctgaaacacaaaataaaataaagttcaagtgttttattgattcaacagttttacaggattcgagcagttatttttcctccaccctcccaggacatggaatacaccaccctctccccccgcacagccttgaacatctgaatgccattggtgatggacatgcttttggtctccacgttccacacagtttcagagcgagccagtctcgggtcggtcagggagatgaaaccctccgggcactcccgcatctgcacctcacagctcaacagctgaggattgtcctcggtggtcgggatcacggttatctggaagaagcagaagagcggcggtgggaatcatagtccgcgaacgggatcggccggtggtgtcgcatcaggccccgcagcagtcgctgccgccgccgctccgtcaagctgctgctcagggggtccgggtccagggactccctcagcatgatgcccacggccctcagcatcagtcgtctggtgcggcgggcgcagcagcgcatgcggatctcgctcaggtcgctgcagtacgtgcaacacagaaccaccaggttgttcaacagtccatagttcaacacgctccagccgaaactcatcgcgggaaggatgctacccacgtggccgtcgtaccagatcctcaggtaaatcaagtggtgccccctccagaacacgctgcccacgtacatgatctccttgggcatgtggcggttcaccacctcccggtaccacatcaccctctggttgaacatgcagccccggatgatcctgcggaaccacagggccagcaccgccccgcccgccatgcagcgaagagaccccgggtcccggcaatggcaatggaggacccaccgctcgtacccgtggatcatctgggagctgaacaagtctatgttggcacagcacaggcatatgctcatgcatctcttcagcactctcaactcctcgggggtcaaaaccatatcccagggcacggggaactcttgcaggacagcgaaccccgcagaacagggcaatcctcgcacagaacttacattgtgcatggacagggtatcgcaatcaggcagcaccgggtgatcctccaccagagaagcgcgggtctcggtctcctcacagcgtggtaagggggccggccgatacgggtgatggcgggacgcggctgatcgtgttcgcgaccgtgtcatgatgcagttgctttcggacattttcgtacttgctgtagcagaacctggtccgggcgctgcacaccgatcgccggcggcggtctcggcgcttggaacgctcggtgttgaaattgtaaaacagccactctctcagaccgtgcagcagatctagggcctcaggagtgatgaagatcccatcatgcctgatggctctgatcacatcgaccaccgtggaatgggccagacccagccagatgatgcaattttgttgggtttcggtgacggcgggggagggaagaacaggaagaaccatgattaacttttaatccaaacggtctcggagtacttcaaaatgaagatcgcggagatggcacctctcgcccccgctgtgttggtggaaaataacagccaggtcaaaggtgatacggttctcgagatgttccacggtggcttccagcaaagcctccacgcgcacatccagaaacaagacaatagcgaaagcgggagggttctctaattcctcaatcatcatgttacactcctgcaccatccccagataattttcatttttccagccttgaatgattcgaactagttcctgaggtaaatccaagccagccatgataaagagctcgcgcagagcgccctccaccggcattcttaagcacaccctcataattccaagatattctgctcctggttcacctgcagcagattgacaagcggaatatcaaaatctctgccgcgatccctgagctcctccctcagcaataactgtaagtactctttcatatcctctccgaaatttttagccataggaccaccaggaataagattagggcaagccacagtacagataaaccgaagtcctccccagtgagcattgccaaatgcaagactgctataagcatgctggctagacccggtgatatcttccagataactggacagaaaatcgcccaggcaatttttaagaaaatcaacaaaagaaaaatcctccaggtggacgtttagagcctcgggaacaacgatgaagtaaatgcaagcggtgcgttccagcatggttagttagctgatctgtagaaaaaacaaaaatgaacattaaaccatgctagcctggcgaacaggtgggtaaatcgttctctccagcaccaggcaggccacggggtctccggcgcgaccctcgtaaaaattgtcgctatgattgaaaaccatcacagagagacgttcccggtggccggcgtgaatgattcgacaagatgaatacacccccggaacattggcgtccgcgagtgaaaaaaagcgcccgaggaagcaataaggcactacaatgctcagtctcaagtccagcaaagcgatgccatgcggatgaagcacaaaattctcaggtgcgtacaaaatgtaattactcccctcctgcacaggcagcaaagcccccgatccctccaggtacacatacaaagcctcagcgtccatagcttaccgagcagcagcacacaacaggcgcaagagtcagagaaaggctgagctctaacctgtccacccgctctctgctcaatatatagcccagatctacactgacgtaaaggccaaagtctaaaaatacccgccaaataatcacacacgcccagcacacgcccagaaaccggtgacacactcaaaaaaatacgcgcacttcctcaaacgcccaaaactgccgtcatttccgggttcccacgctacgtcatcaaaacacgactttcaaattccgtcgaccgttaaaaacgtcacccgccccgcccctaacggtcgcccgtctctcagccaatcagcgccccgcatccccaaattcaaacacctcatttgcatattaacgcgcacaaaaagtttgaggtatattattgatgatgg

SEQ ID NO: 11 - DNA, 31867 bp, synthetic construct
  misc_feature 1..31867: note = Description of Artificial Sequence: Synthetic polynucleotide
  source 1..31867: mol_type = other DNA
ccatcttcaataatatacctcaaactttttgtgcgcgttaatatgcaaatgaggcgtttgaatttggggaggaagggcggtgattggtcgagggatgagcgaccgttaggggcggggcgagtgacgttttgatgacgtggttgcgaggaggagccagtttgcaagttctcgtgggaaaagtgacgtcaaacgaggtgtggtttgaacacggaaatactcaattttcccgcgctctctgacaggaaatgaggtgtttctgggcggatgcaagtgaaaacgggccattttcgcgcgaaaactgaatgaggaagtgaaaatctgagtaatttcgcgtttatggcagggaggagtatttgccgagggccgagtagactttgaccgattacgtgggggtttcgattaccgtgtttttcacctaaatttccgcgtacggtgtcaaagtccggtgtttttacgtaggtgtcagctgatcgccagggtatttaaacctgcgctctccagtcaagaggccactcttgagtgccagcgagaagagttttctcctccgcgccgcgagtcagatctacactttgaaagtagggataacagggtaatgacattgattattgactagttgttaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggaacgccatccacgctgttttgacctccatagaagacagcgatcgcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctttacaagtagtgagtttaaactcccatttaaatgtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataactataacggtcctaaggtagcgagtgagtagtgttctggggcgggggaggacctgcatgagggccagaataactgaaatctgtgcttttctgtgtgttgcagcagcatgagcggaagcggctcctttgagggaggggtattcagcccttatctgacggggcgtctcccctcctgggcgggagtgcgtcagaatgtgatgggatccacggtggacggccggcccgtgcagcccgcgaactcttcaaccctgacctatgcaaccctgagctcttcgtcgttggacgcagctgccgccgcagctgctgcatctgccgccagcgccgtgcgcggaatggccatgggcgccggctactacggcactctggtggccaactcgagttccaccaataatcccgccagcctgaacgaggagaagctgttgctgctgatggcccagctcgaggccttgacccagcgcctgggcgagctgacccagcaggtggctcagctgcaggagcagacgcgggccgcggttgccacggtgaaatccaaataaaaaatgaatcaataaataaacggagacggttgttgattttaacacagagtctgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcatggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcctttgtgcccgcccaggttttccatgcactcatccatgatgatggcgatgggcccgtgggcggcggcctgggcaaagacgtttcgggggtcggacacatcatagttgtggtcctgggtgaggtcatcataggccattttaatgaatttggggcggagggtgccggactgggggacaaaggtaccctcgatcccgggggcgtagttcccctcacagatctgcatctcccaggctttgagctcggagggggggatcatgtccacctgcggggcgataaagaacacggtttccggggcgggggagatgagctgggccgaaagcaagttccggagcagctgggacttgccgcagccggtggggccgtagatgaccccgatgaccggctgcaggtggtagttgagggagagacagctgccgtcctcccggaggaggggggccacctcgttcatcatctcgcgcacgtgcatgttctcgcgcaccagttccgccaggaggcgctctccccccagggataggagctcctggagcgaggcgaagtttttcagcggcttgagtccgtcggccatgggcattttggagagggtttgttgcaagagttccaggcggtcccagagctcggtgatgtgctctacggcatctcgatccagcagacctcctcgtttcgcgggttgggacggctgcgggagtagggcaccagacgatgggcgtccagcgcagccagggtccggtccttccagggtcgcagcgtccgcgtcagggtggtctccgtcacggtgaaggggtgcgcgccgggctgggcgcttgcgagggtgcgcttcaggctcatccggctggtcgaaaaccgctcccgatcggcgccctgcgcgtcggccaggtagcaattgaccatgagttcgtagttgagcgcctcggccgcgtggcctttggcgcggagcttacctttggaagtctgcccgcaggcgggacagaggagggacttgagggcgtagagcttgggggcgaggaagacggactcgggggcgtaggcgtccgcgccgcagtgggcgcagacggtctcgcactccacgagccaggtgaggtcgggctggtcggggtcaaaaaccagtttcccgccgttctttttgatgcgtttcttacctttggtctccatgagctcgtgtccccgctgggtgacaaagaggctgtccgtgtccccgtagaccgactttatgggccggtcctcgagcggtgtgccgcggtcctcctcgtagaggaaccccgcccactccgagacgaaagcccgggtccaggccagcacgaaggaggccacgtgggacgggtagcggtcgttgtccaccagcgggtccaccttttccagggtatgcaaacacatgtccccctcgtccacatccaggaaggtgattggcttgtaagtgtaggccacgtgaccgggggtcccggccgggggggtataaaagggtgcgggtccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccggcggagatgcctttcaagagcccctcgtccatctggtcagaaaagacgatctttttgttgtcgagcttggtggcgaaggagccgtagagggcgttggagaggagcttggcgatggagcgcatggtctggtttttttccttgtcggcgcgctccttggcggcgatgttgagctgcacgtactcgcgcgccacgcacttccattcggggaagacggtggtcagctcgtcgggcacgattctgacctgccagccccgattatgcagggtgatgaggtccacactggtggccacctcgccgcgcaggggctcattagtccagcagaggcgtccgcccttgcgcgagcagaaggggggcagggggtccagcatgacctcgtcgggggggtcggcatcgatggtgaagatgccgggcaggaggtcggggtcaaagtagctgatggaagtggccagatcgtccagggcagcttgccattcgcgcacggccagcgcgctctcgtagggactgaggggcgtgccccagggcatgggatgggtaagcgcggaggcgtacatgccgcagatgtcgtagacgtagaggggctcctcgaggatgccgatgtaggtggggtagcagcgccccccgcggatgctggcgcgcacgtagtcatacagctcgtgcgagggggcgaggagccccgggcccaggttggtgcgactgggcttttcggcgcggtagacgatctggcggaaaatggcatgcgagttggaggagatggtgggcctttggaagatgttgaagtgggcgtggggcagtccgaccgagtcgcggatgaagtgggcgtaggagtcttgcagcttggcgacgagctcggcggtgactaggacgtccagagcgcagtagtcgagggtctcctggatgatgtcatacttgagctgtcccttttgtttccacagctcgcggttgagaaggaactcttcgcggtccttccagtactcttcgagggggaacccgtcctgatctgcacggtaagagcctagcatgtagaactggttgacggccttgtaggcgcagcagcccttctccacggggagggcgtaggcctgggcggccttgcgcagggaggtgtgcgtgagggcgaaagtgtccctgaccatgaccttgaggaactggtgcttgaagtcgatatcgtcgcagcccccctgctcccagagctggaagtccgtgcgcttcttgtaggcggggttgggcaaagcgaaagtaacatcgttgaagaggatcttgcccgcgcggggcataaagttgcgagtgatgcggaaaggttggggcacctcggcccggttgttgatgacctgggcggcgagcacgatctcgtcgaagccgttgatgttgtggcccacgatgtagagttccacgaatcgcggacggcccttgacgtggggcagtttcttgagctcctcgtaggtgagctcgtcggggtcgctgagcccgtgctgctcgagcgcccagtcggcgagatgggggttggcgcggaggaaggaagtccagagatccacggccagggcggtttgcagacggtcccggtactgacggaactgctgcccgacggccattttttcgggggtgacgcagtagaaggtgcgggggtccccgtgccagcgatcccatttgagctggagggcgagatcgagggcgagctcgacgagccggtcgtccccggagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccattggtggcgacgtcgatggcttgcagggtcccgtgcccctggggtgtgaccaccgtcccccgtttcttcttgggcggctggggcgacgggggcggtgcctcttccatggttagaagcggcggcgaggacgcgcgccgggcggcaggggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcttgaaggtctccgcggccggcgcgctccacggtggccgcgaggtcgttggagatgcggcccatgagctgcgagaaggcgttcatgcccgcctcgttccagacgcggctgtagaccacgacgccctcgggatcgcgggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaaacgttccatggcctcgtaaaagtccacggcgaagttgaaaaactgggagttgcgcgccgagacggtcaactcctcctccagaagacggatgagctcggcgatggtggcgcgcacctcgcgctcgaaggcccccgggagttcctccacttcctcttcttcctcctccactaacatctcttctacttcctcctcaggcggcagtggtggcgggggagggggcctgcgtcgccggcggcgcacgggcagacggtcgatgaagcgctcgatggtctcgccgcgccggcgtcgcatggtctcggtgacggcgcgcccgtcctcgcggggccgcagcgtgaagacgccgccgcgcatctccaggtggccgggggggtccccgttgggcagggagagggcgctgacgatgcatcttatcaattgccccgtagggactccgcgcaaggacctgagcgtctcgagatccacgggatctgaaaaccgctgaacgaaggcttcgagccagtcgcagtcgcaaggtaggctgagcacggtttcttctggcgggtcatgttggttgggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccaggtccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcttgctggatctgggtgagggtggtctggaagtcatcaaagtcgacgaagcggtggtaggctccggtgttgatggtgtaggagcagttggccatgacggaccagttgacggtctggtggcccggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtattggcactcccgtctcgacccaagcctgcaccaaccctccaggatacggaggcgggtcgttttgcaacttttttttggaggccggatgagactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagaccccatagccagccgacttctccagttacggagcgagcccctcttttgttttgtttgtttttgccagatgcatcccgtactgcggcagatgcgcccccaccaccctccaccgcaacaacagccccctccacagccggcgcttctgcccccgccccagcagcaacttccagccacgaccgccgcggccgccgtgagcggggctggacagagttatgatcaccagctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaagcgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagtttgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgtgcggtgagcgccagcaggcggcgcgagctgagcgaccaggagctgatgcatagtctgcagcgggccctgaccggggccgggaccgagggggagagctactttgacatgggcgcggacctgcactggcagcccagccgccgggccttggaggcggcggcaggaccctacgtagaagaggtggacgatgaggtggacgaggagggcgagtacctggaagactgatggcgcgaccgtatttttgctagatgcaacaacaacagccacctcctgatcccgcgatgcgggcggcgctgcagagccagccgtccggcattaactcctcggacgattggacccaggccatgcaacgcatcatggcgctgacgacccgcaaccccgaagcctttagacagcagccccaggccaaccggctctcggccatcctggaggccgtggtgccctcgcgctccaaccccacgcacgagaaggtcctggccatcgtgaacgcgctggtggagaacaaggccatccgcggcgacgaggccggcctggtgtacaacgcgctgctggagcgcgtggcccgctacaacagcaccaacgtgcagaccaacctggaccgcatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggctttcaagaacttgcagggcctgtggggcgtgcaggccccggtcggggaccgcgcgacggtgtcgagcctgctgacgccgaactcgcgcctgctgctgctgctggtggcccccttcacggacagcggcagcatcaaccgcaactcgtacctgggctacctgattaacctgtaccgcgaggccatcggccaggcgcacgtggacgagcagacctaccaggagatcacccacgtgagccgcgccctgggccaggacgacccgggcaacctggaagccaccctgaactttttgctgaccaaccggtcgcagaagatcccgccccagtacgcgctcagcaccgaggaggagcgcatcctgcgttacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctgaatccccactggctcccgccgccggggttctacacgggcgagtacgacatgcccgaccccaatgacgggttcctgtgggacgatgtggacagcagcgtgttctccccccgaccgggtgctaacgagcgccccttgtggaagaaggaaggcagcgaccgacgcccgtcctcggcgctgtccggccgcgagggtgctgccgcggcggtgcccgaggccgccagtcctttcccgagcttgcccttctcgctgaacagtatccgcagcagcgagctgggcaggatcacgcgcccgcgcttgctgggcgaagaggagtacttgaatgactcgctgttgagacccgagcgggagaagaacttccccaataacgggatagaaagcctggtggacaagatgagccgctggaagacgtatgcgcaggagcacagggacgatccccgggcgtcgcagggggccacgagccggggcagcgccgcccgtaaacgccggtggcacgacaggcagcggggacagatgtgggacgatgaggactccgccgacgacagcagcgtgttggacttgggtgggagtggtaacccgttcgctcacctgcgcccccgtatcgggcgcatgatgtaagagaaaccgaaaataaatgatactcaccaaggccatggcgaccagcgtgcgttcgtttcttctctgttgttgttgtatctagtatgatgaggcgtgcgtacccggagggtcctcctccctcgtacgagagcgtgatgcagcaggcgatggcggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggccagctgaaaaccatcatgcacaccaacatgcccaacgtgaacgagttcatgtacagcaacaagttcaaggcgcgggtgatggtctcccgcaagacccccaatggggtgacagtgacagaggattatgatggtagtcaggatgagctgaagtatgaatgggtggaatttgagctgcccgaaggcaacttctcggtgaccatgaccatcgacctgatgaacaacgccatcatcgacaattacttggcggtggggcggcagaacggggtgctggagagcgacatcggcgtgaagttcgacactaggaacttcaggctgggctgggaccccgtgaccgagctggtcatgcccggggtgtacaccaacgaggctttccatcccgatattgtcttgctgcccggctgcggggtggacttcaccgagagccgcctcagcaacctgctgggcattcgcaagaggcagcccttccaggaaggcttccagatcatgtacgaggatctggaggggggcaacatccccgcgctcctggatgtcgacgcctatgagaaaagcaaggaggatgcagcagctgaagcaactgcagccgtagctaccgcctctaccgaggtcaggggcgataattttgcaagcgccgcagcagtggcagcggccgaggcggctgaaaccgaaagtaagatagtcattcagccggtggagaaggatagcaagaacaggagctacaacgtactaccggacaagataaacaccgcctaccgcagctggtacctagcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgcttacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcggtcgcgcaccaccgtcgacgacgtgatcgaccaggtggtggccgacgcgcgcaactacacccccgccgccgcgcccgtctccaccgtggacgccgtcatcgacagcgtggtggccgacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcttcaggcgccagcgccggcaggacccggagacgcgcggccacggcggcggcagcggccatcgccagcatgtcccgcccgcggcgagggaacgtgtactgggtgcgcgacgccgccaccggtgtgcgcgtgcccgtgcgcacccgcccccctcgcacttgaagatgttcacttcgcgatgttgatgtgtcccagcggcgaggaggatgtccaagcgcaaattcaaggaagagatgctccaggtcatcgcgcctgagatctacggccctgcggtggtgaaggaggaaagaaagccccgcaaaatcaagcgggtcaaaaaggacaaaaaggaagaagaaagtgatgtggacggattggtggagtttgtgcgcgagttcgccccccggcggcgcgtgcagtggcgcgggcggaaggtgcaaccggtgctgagacccggcaccaccgtggtcttcacgcccggcgagcgctccggcaccgcttccaagcgctcctacgacgaggtgtacggggatgatgatattctggagcaggcggccgagcgcctgggcgagtttgcttacggcaagcgcagccgttccgcaccgaaggaagaggcggtgtccatcccgctggaccacggcaaccccacgccgagcctcaagcccgtgaccttgcagcaggtgctgccgaccgcggcgccgcgccgggggttcaagcgcgagggcgaggatctgtaccccaccatgcagctgatggtgcccaagcgccagaagctggaagacgtgctggagaccatgaaggtggacccggacgtgcagcccgaggtcaaggtgcggcccatcaagcaggtggccccgggcctgggcgtgcagaccgtggacatcaagattcccacggagcccatggaaacgcagaccgagcccatgatcaagcccagcaccagcaccatggaggtgcagacggatccctggatgccatcggctcctagtcgaagaccccggcgcaagtacggcgcggccagcctgctgatgcccaactacgcgctgcatccttccatcatccccacgccgggctaccgcggcacgcgcttctaccgcggtcataccagcagccgccgccgcaagaccaccactcgccgccgccgtcgccgcaccgccgctgcaaccacccctgccgccctggtgcggagagtgtaccgccgcggccgcgcacctctgaccctgccgcgcgcgcgctaccacccgagcatcgccatttaaactttcgcctgctttgcagatcaatggccctcacatgccgccttcgcgttcccattacgggctaccgaggaagaaaaccgcgccgtagaaggctggcggggaacgggatgcgtcgccaccaccaccggcggcggcgcgccatcagcaagcggttggggggaggcttcctgcccgcgctgatccccatcatcgccgcggcgatcggggcgatccccggcattgcttccgtggcggtgcaggcctctcagcgccactgagacacacttggaaacatcttgtaataaaccaatggactctgacgctcctggtcctgtgatgtgttttcgtagacagatggaagacatcaatttttcgtccctggctccgcgacacggcacgcggccgttcatgggcacctggagcgacatcggcaccagccaactgaacgggggcgccttcaattggagcagtctctggagcgggcttaagaatttcgggtccacgcttaaaacctatggcagcaaggcgtggaacagcaccacagggcaggcgctgagggataagctgaaagagcagaacttccagcagaaggtggtcgatgggctcgcctcgggcatcaacggggtggtggacctggccaaccaggccgtgcagcggcagatcaacagccgcctggacccggtgccgcccgccggctccgtggagatgccgcaggtggaggaggagctgcctcccctggacaagcggggcgagaagcgaccccgccccgatgcggaggagacgctgctgacgcacacggacgagccgcccccgtacgaggaggcggtgaaactgggtctgcccaccacgcggcccatcgcgcccctggccaccggggtgctgaaacccgaaaagcccgcgaccctggacttgcctcctccccagccttcccgcccctctacagtggctaagcccctgccgccggtggccgtggcccgcgcgcgacccgggggcaccgcccgccctcatgcgaactggcagagcactctgaacagcatcgtgggtctgggagtgcagagtgtgaagcgccgccgctgctattaaacctaccgtagcgcttaacttgcttgtctgtgtgtgtatgtattatgtcgccgccgccgctgtccaccagaaggaggagtgaagaggcgcgtcgccgagttgcaagatggccaccccatcgatgctgccccagtgggcgtacatgcacatcgccggacaggacgcttcggagtacctgagtccgggtctggtgcagtttgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccctagcttcaaaccctactccggcaccgcctacaacagtctggcccccaagggagcacccaacacttgtcagtggacatataaagccgatggtgaaactgccacagaaaaaacctatacatatggaaatgcacccgtgcagggcattaacatcacaaaagatggtattcaacttggaactgacaccgatgatcagccaatctacgcagataaaacctatcagcctgaacctcaagtgggtgatgctgaatggcatgacatcactggtactgatgaaaagtatggaggcagagctcttaagcctgataccaaaatgaagccttgttatggttcttttgccaagcctactaataaagaaggaggtcaggcaaatgtgaaaacaggaacaggcactactaaagaatatgacatagacatggctttctttgacaacagaagtgcggctgctgctggcctagctccagaaattgttttgtatactgaaaatgtggatttggaaactccagatacccatattgtatacaaagcaggcacagatgacagcagctcttctattaatttgggtcagcaagccatgcccaacagacctaactacattggtttcagagacaactttatcgggctcatgtactacaacagcactggcaatatgggggtgctggccggtcaggcttctcagctgaatgctgtggttgacttgcaagacagaaacaccgagctgtcctaccagctcttgcttgactctctgggtgacagaacccggtatttcagtatgtggaatcaggcggtggacagctatgatcctgatgtgcgcattattgaaaatcatggtgtggaggatgaacttcccaactattgtttccctctggatgctgttggcagaacagatacttatcagggaattaaggctaatggaactgatcaaaccacatggaccaaagatgacagtgtcaatgatgctaatgagataggcaagggtaatccattcgccatggaaatcaacatccaagccaacctgtggaggaacttcctctacgccaacgtggccctgtacctgcccgactcttacaagtacacgccggccaatgttaccctgcccaccaacaccaacacctacgattacatgaacggccgggtggtggcgccctcgctggtggactcctacatcaacatcggggcgcgctggtcgctggatcccatggacaacgtgaaccccttcaaccaccaccgcaatgcggggctgcgctaccgctccatgctcctgggcaacgggcgctacgtgcccttccacatccaggtgccccagaaatttttcgccatcaagagcctcctgctcctgcccgggtcctacacctacgagtggaacttccgcaaggacgtcaacatgatcctgcagagctccctcggcaacgacctgcgcacggacggggcctccatctccttcaccagcatcaacctctacgccaccttcttccccatggcgcacaacacggcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggctggtccttcacgcgtctcaagaccaaggagacgccctcgctgggctccgggttcgacccctacttcgtctactcgggctccatcccctacctcgacggcaccttctacctcaaccacaccttcaagaaggtctccatcaccttcgactcctccgtcagctggcccggcaacgaccggctcctgacgcccaacgagttcgaaatcaagcgcaccgtcgacggcgagggctacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtggtggacgaggtcaactacaaggactaccaggccgtcaccctggcctaccagcacaacaactcgggcttcgtcggctacctcgcgcccaccatgcgccagggccagccctaccccgccaactacccctacccgctcatcggcaagagcgccgtcaccagcgtcacccagaaaaagttcctctgcgacagggtcatgtggcgcatccccttctccagcaacttcatgtccatgggcgcgctcaccgacctcggccagaacatgctctatgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacccccttctcggccggtaacgccaccacctaagctcttgcttcttgcaagccatggccgcgggctccggcgagcaggagctcagggccatcatccgcgacctgggctgcgggccctacttcctgggcaccttcgataagcgcttcccgggattcatggccccgcacaagctggcctgcgccatcgtcaacacggccggccgcgagaccgggggcgagcactggctggccttcgcctggaacccgcgctcgaacacctgctacctcttcgaccccttcgggttctcggacgagcgcctcaagcagatctaccagttcgagtacgagggcctgctgcgccgcagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaactcccactccgcctactttcgctcccaccgcgcgcgcatcgagaaggccaccgccttcgaccgcatgaatcaagacatgtaaaccgtgtgtgtatgttaaatgtctttaataaacagcactttcatgttacacatgcatctgagatgatttatttagaaatcgaaagggttctgccgggtctcggcatggcccgcgggcagggacacgttgcggaactggtacttggccagccacttgaactcggggatcagcagtttgggcagcggggtgtcggggaaggagtcggtccacagcttccgcgtcagttgcagggcgcccagcaggtcgggcgcggagatcttgaaatcgcagttgggacccgcgttctgcgcgcgggagttgcggtacacggggttgcagcactggaacaccatcagggccgggtgcttcacgctcgccagcaccgtcgcgtcggtgatgctctccacgtcgaggtcctcggcgttggccatcccgaagggggtcatcttgcaggtctgccttcccatggtgggcacgcacccgggcttgtggttgcaatcgcagtgcagggggatcagcatcatctgggcctggtcggcgttcatccccgggtacatggccttcatgaaagcctccaattgcctgaacgcctgctgggccttggctccctcggtgaagaagaccccgcaggacttgctagagaactggttggtggcgcacccggcgtcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcatgtgctccttctggatcatggtggtcccgtgcaggcaccgcagcttgccctcggcctcggtgcacccgtgcagccacagcgcgcacccggtgcactcccagttcttgtgggcgatctgggaatgcgcgtgcacgaagccctgcaggaagcggcccatcatggtggtcagggtcttgttgctagtgaaggtcagcggaatgccgcggtgctcctcgttgatgtacaggtggcagatgcggcggtacacctcgccctgctcgggcatcagctggaagttggctttcaggtcggtctccacgcggtagcggtccatcagcatagtcatgatttccatacccttctcccaggccgagacgatgggcaggctcatagggttcttcaccatcatcttagcgctagcagccgcggccagggggtcgctctcgtccagggtctcaaagctccgcttgccgtccttctcggtgatccgcaccggggggtagctgaagcccacggccgccagctcctcctcggcctgtctttcgtcctcgctgtcctggctgacgtcctgcaggaccacatgcttggtcttgcggggtttcttcttgggcggcagcggcggcggagatgttggagatggcgagggggagcgcgagttctcgctcaccactactatctcttcctcttcttggtccgaggccacgcggcggtaggtatgtctcttcgggggcagaggcggaggcgacgggctctcgccgccgcgacttggcggatggctggcagagccccttccgcgttcgggggtgcgctcccggcggcgctctgactgacttcctccgcggccggccattgtgttctcctagggaggaacaacaagcatggagactcagccatcgccaacctcgccatctgcccccaccgccgacgagaagcagcagcagcagaatgaaagcttaaccgccccgccgcccagccccgccacctccgacgcggccgtcccagacatgcaagagatggaggaatccatcgagattgacctgggctatgtgacgcccgcggagcacgaggaggagctggcagtgcgcttttcacaagaagagatacaccaagaacagccagagcaggaagcagagaatgagcagagtcaggctgggctcgagcatgacggcgactacctccacctgagcgggggggaggacgcgctcatcaagcatctggcccggcaggccaccatcgtcaaggatgcgctgctcgaccgcaccgaggtgcccctcagcgtggaggagctcagccgcgcctacgagttgaacctcttctcgccgcgcgtgccccccaagcgccagcccaatggcacctgcgagcccaacccgcgcctcaacttctacccggtcttcgcggtgcccgaggccctggccacctaccacatctttttcaagaaccaaaagatccccgtctcctgccgcgccaaccgcacccgcgccgacgcccttttcaacctgggtcccggcgcccgcctacctgatatcgcctccttggaagaggttcccaagatcttcgagggtctgggcagcgacgagactcgggccgcgaacgctctgcaaggagaaggaggagagcatgagcaccacagcgccctggtcgagttggaaggcgacaacgcgcggctggcggtgctcaaacgcacggtcgagctgacccatttcgcctacccggctctgaacctgccccccaaagtcatgagcgcggtcatggaccaggtgctcatcaagcgcgcgtcgcccatctccgaggacgagggcatgcaagactccgaggagggcaagcccgtggtcagcgacgagcagctggcccggtggctgggtcctaatgctagtccccagagtttggaagagcggcgcaaactcatgatggccgtggtcctggtgaccgtggagctggagtgcctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcttgcacgagaaccgcctggggcagaacgtgctgcacaccaccctgcgcggggaggcccggcgcgactacatccgcgactgcgtctacctctacctctgccacacctggcagacgggcatgggcgtgtggcagcagtgtctggaggagcagaacctgaaagagctctgcaagctcctgcagaagaacctcaagggtctgtggaccgggttcgacgagcgcaccaccgcctcggacctggccgacctcattttccccgagcgcctcaggctgacgctgcgcaacggcctgcccgactttatgagccaaagcatgttgcaaaactttcgctctttcatcctcgaacgctccggaatcctgcccgccacctgctccgcgctgccctcggacttcgtgccgctgaccttccgcgagtgccccccgccgctgtggagccactgctacctgctgcgcctggccaactacctggcctaccactcggacgtgatcgaggacgtcagcggcgagggcctgctcgagtgccactgccgctgcaacctctgcacgccgcaccgctccctggcctgcaacccccagctgctgagcgagacccagatcatcggcaccttcgagttgcaagggcccagcgaaggcgagggttcagccgccaaggggggtctgaaactcaccccggggctgtggacctcggcctacttgcgcaagttcgtgcccgaggactaccatcccttcgagatcaggttctacgaggaccaatcccatccgcccaaggccgagctgtcggcctgcgtcatcacccagggggcgatcctggcccaattgcaagccatccagaaatcccgccaagaattcttgctgaaaaagggccgcggggtctacctcgacccccagaccggtgaggagctcaaccccggcttcccccaggatgccccgaggaaacaagaagctgaaagtggagctgccgcccgtggaggatttggaggaagactgggagaacagcagtcaggcagaggaggaggagatggaggaagactgggacagcactcaggcagaggaggacagcctgcaagacagtctggaggaagacgaggaggaggcagaggaggaggtggaagaagcagccgccgccagaccgtcgtcctcggcgggggagaaagcaagcagcacggataccatctccgctccgggtcggggtcccgctcgaccacacagtagatgggacgagaccggacgattcccgaaccccaccacccagaccggtaagaaggagcggcagggatacaagtcctggcgggggcacaaaaacgccatcgtctcctgcttgcaggcctgcgggggcaacatctccttcacccggcgctacctgctcttccaccgcggggtgaactttccccgcaacatcttgcattactaccgtcacctccacagcccctactacttccaagaagaggcagcagcagcagaaaaagaccagcagaaaaccagcagctagaaaatccacagcggcggcagcaggtggactgaggatcgcggcgaacgagccggcgcaaacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccagtcgcagaaaaaggcgggaattacgtcacctgtgcccttcgccctagccgcctccacccatcatcatgagcaaagagattcccacgccttacatgtggagctaccagccccagatgggcctggccgccggtgccgcccaggactactccacccgcatgaattggctcagcgccgggcccgcgatgatctcacgggtgaatgacatccgcgcccaccgaaaccagatactcctagaacagtcagcgctcaccgccacgccccgcaatcacctcaatccgcgtaattggcccgccgccctggtgtaccaggaaattccccagcccacgaccgtactacttccgcgagacgcccaggccgaagtccagctgactaactcaggtgtccagctggcgggcggcgccaccctgtgtcgtcaccgccccgctcagggtataaagcggctggtgatccggggcagaggcacacagctcaacgacgaggtggtgagctcttcgctgggtctgcgacctgacggagtcttccaactcgccggatcggggagatcttccttcacgcctcgtcaggccgtcctgactttggagagttcgtcctcgcagccccgctcgggtggcatcggcactctccagttcgtggaggagttcactccctcggtctacttcaaccccttctccggctcccccggccactacccggacgagttcatcccgaacttcgacgccatcagcgagtcggtggacggctacgattgaatgtcccatggtggcgcagctgacctagctcggcttcgacacctggaccactgccgccgcttccgctgcttcgctcgggatctcgccgagtttgcctactttgagctgcccgaggagcaccctcagggcccggcccacggagtgcggatcgtcgtcgaagggggcctcgactcccacctgcttcggatcttcagccagcgtccgatcctggtcgagcgcgagcaaggacagacccttctgactctgtactgcatctgcaaccaccccggcctgcatgaaagtctttgttgtctgctgtgtactgagtataataaaagctgagatcagcgactactccggacttccgtgtgttcctgaatccatcaaccagtctttgttcttcaccgggaacgagaccgagctccagctccagtgtaagccccacaagaagtacctcacctggctgttccagggctccccgatcgccgttgtcaaccactgcgacaacgacggagtcctgctgagcggccctgccaaccttactttttccacccgcagaagcaagctccagctcttccaacccttcctccccgggacctatcagtgcgtctcgggaccctgccatcacaccttccacctgatcccgaataccacagcgtcgctccccgctactaacaaccaaactaacctccaccaacgccaccgtcgcgacggccacaatacatgcccatattagactatgaggccgagccacagcgacccatgctccccgctattagttacttcaatctaaccggcggagatgactgacccactggccaacaacaacgtcaacgaccttctcctggacatggacggccgcgcctcggagcagcgactcgcccaacttcgcattcgccagcagcaggagagagccgtcaaggagctgcaggatgcggtggccatccaccagtgcaagagaggcatcttctgcctggtgaaacaggccaagatctcctacgaggtcactccaaacgaccatcgcctctcctacgagctcctgcagcagcgccagaagttcacctgcctggtcggagtcaaccccatcgtcatcacccagcagtctggcgataccaaggggtgcatccactgctcctgcgactcccccgactgcgtccacactctgatcaagaccctctgcggcctccgcgacctcctccccatgaactaatcacccccttatccagtgaaataaagatcatattgatgatgattttacagaaataaaaaataatcatttgatttgaaataaagatacaatcatattgatgatttgagtttaacaaaaaaataaagaatcacttacttgaaatctgataccaggtctctgtccatgttttctgccaacaccacttcactcccctcttcccagctctggtactgcaggccccggcgggctgcaaacttcctccacacgctgaaggggatgtcaaattcctcctgtccctcaatcttcattttatcttctatcagatgtccaaaaagcgcgtccgggtggatgatgacttcgaccccgtctacccctacgatgcagacaacgcaccgaccgtgcccttcatcaacccccccttcgtctcttcagatggattccaagagaagcccctgggggtgttgtccctgcgactggccgaccccgtcaccaccaagaacggggaaatcaccctcaagctgggagagggggtggacctcgattcctcgggaaaactcatctccaacacggccaccaaggccgccgcccctctcagtttttccaacaacaccatttcccttaacatggatcaccccttttacactaaagatggaaaattatccttacaagtttctccaccattaaatatactgagaacaagcattctaaacacactagctttaggttttggatcaggtttaggactccgtggctctgccttggcagtacagttagtctctccacttacatttgatactgatggaaacataaagcttaccttagacagaggtttgcatgttacaacaggagatgcaattgaaagcaacataagctgggctaaaggtttaaaatttgaagatggagccatagcaaccaacattggaaatgggttagagtttggaagcagtagtacagaaacaggtgttgatgatgcttacccaatccaagttaaacttggatctggccttagctttgacagtacaggagccataatggctggtaacaaagaagacgataaactcactttgtggacaacacctgatccatcaccaaactgtcaaatactcgcagaaaatgatgcaaaactaacactttgcttgactaaatgtggtagtcaaatactggccactgtgtcagtcttagttgtaggaagtggaaacctaaaccccattactggcaccgtaagcagtgctcaggtgtttctacgttttgatgcaaacggtgttcttttaacagaacattctacactaaaaaaatactgggggtataggcagggagatagcatagatggcactccatataccaatgctgtaggattcatgcccaatttaaaagcttatccaaagtcacaaagttctactactaaaaataatatagtagggcaagtatacatgaatggagatgtttcaaaacctatgcttctcactataaccctcaatggtactgatgacagcaacagtacatattcaatgtcattttcatacacctggactaatggaagctatgttggagcaacatttggggctaactcttataccttctcatacatcgcccaagaatgaacactgtatcccaccctgcatgccaacccttcccaccccactctgtggaacaaactctgaaacacaaaataaaataaagttcaagtgttttattgattcaacagttttacaggattcgagcagttatttttcctccaccctcccaggacatggaatacaccaccctctccccccgcacagccttgaacatctgaatgccattggtgatggacatgcttttggtctccacgttccacacagtttcagagcgagccagtctcgggtcggtcagggagatgaaaccctccgggcactcccgcatctgcacctcacagctcaacagctgaggattgtcctcggtggtcgggatcacggttatctggaagaagcagaagagcggcggtgggaatcatagtccgcgaacgggatcggccggtggtgtcgcatcaggccccgcagcagtcgctgccgccgccgctccgtcaagctgctgctcagggggtccgggtccagggactccctcagcatgatgcccacggccctcagcatcagtcgtctggtgcggcgggcgcagcagcgcatgcggatctcgctcaggtcgctgcagtacgtgcaacacagaaccaccaggttgttcaacagtccatagttcaacacgctccagccgaaactcatcgcgggaaggatgctacccacgtggccgtcgtaccagatcctcaggtaaatcaagtggtgccccctccagaacacgctgcccacgtacatgatctccttgggcatgtggcggttcaccacctcccggtaccacatcaccctctggttgaacatgcagccccggatgatcctgcggaaccacagggccagcaccgccccgcccgccatgcagcgaagagaccccgggtcccggcaatggcaatggaggacccaccgctcgtacccgtggatcatctgggagctgaacaagtctatgttggcacagcacaggcatatgctcatgcatctcttcagcactctcaactcctcgggggtcaaaaccatatcccagggcacggggaactcttgcaggacagcgaaccccgcagaacagggcaatcctcgcacagaacttacattgtgcatggacagggtatcgcaatcaggcagcaccgggtgatcctccaccagagaagcgcgggtctcggtctcctcacagcgtggtaagggggccggccgatacgggtgatggcgggacgcggctgatcgtgttcgcgaccgtgtcatgatgcagttgctttcggacattttcgtacttgctgtagcagaacctggtccgggcgctgcacaccgatcgccggcggcggtctcggcgcttggaacgctcggtgttgaaattgtaaaacagccactctctcagaccgtgcagcagatctagggcctcaggagtgatgaagatcccatcatgcctgatggctctgatcacatcgaccaccgtggaatgggccagacccagccagatgatgcaattttgttgggtttcggtgacggcgggggagggaagaacaggaagaaccatgattaacttttaatccaaacggtctcggagtacttcaaaatgaagatcgcggagatggcacctctcgcccccgctgtgttggtggaaaataacagccaggtcaaaggtgatacggttctcgagatgttccacggtggcttccagcaaagcctccacgcgcacatccagaaacaagacaatagcgaaagcgggagggttctctaattcctcaatcatcatgttacactcctgcaccatccccagataattttcatttttccagccttgaatgattcgaactagttcgtgaggtaaatccaagccagccatgataaagagctcgcgcagagcgccctccaccggcattcttaagcacaccctcataattccaagatattctgctcctggttcacctgcagcagattgacaagcggaatatcaaaatctctgccgcgatccctgagctcctccctcagcaataactgtaagtactctttcatatcctctccgaaatttttagccataggaccaccaggaataagattagggcaagccacagtacagataaaccgaagtcctccccagtgagcattgccaaatgcaagactgctataagcatgctggctagacccggtgatatcttccagataactggacagaaaatcgcccaggcaatttttaagaaaatcaacaaaagaaaaatcctccaggtggacgtttagagcctcgggaacaacgatgaagtaaatgcaagcggtgcgttccagcatggttagttagctgatctgtagaaaaaacaaaaatgaacattaaaccatgctagcctggcgaacaggtgggtaaatcgttctctccagcaccaggcaggccacggggtctccggcgcgaccctcgtaaaaattgtcgctatgattgaaaaccatcacagagagacgttcccggtggccggcgtgaatgattcgacaagatgaatacacccccggaacattggcgtccgcgagtgaaaaaaagcgcccgaggaagcaataaggcactacaatgctcagtctcaagtccagcaaagcgatgccatgcggatgaagcacaaaattctcaggtgcgtacaaaatgtaattactcccctcctgcacaggcagcaaagcccccgatccctccaggtacacatacaaagcctcagcgtccatagcttaccgagcagcagcacacaacaggcgcaagagtcagagaaaggctgagctctaacctgtccacccgctctctgctcaatatatagcccagatctacactgacgtaaaggccaaagtctaaaaatacccgccaaataatcacacacgcccagcacacgcccagaaaccggtgacacactcaaaaaaatacgcgcacttcctcaaacgcccaaaactgccgtcatttccgggttcccacgctacgtcatcaaaacacgactttcaaattccgtcgaccgttaaaaacgtcacccgccccgcccctaacggtcgcccgtctctcagccaatcagcgccccgcatccccaaattcaaacacctcatttgcatattaacgcgcacaaaaagtttgaggtatattattgatgatgg

SEQ ID NO: 12 - DNA, 32788 bp, synthetic construct
  misc_feature 1..32788: note = Description of Artificial Sequence: Synthetic polynucleotide
  source 1..32788: mol_type = other DNA
ccatcttcaataatatacctcaaactttttgtgcgcgttaatatgcaaatgaggcgtttgaatttggggaggaagggcggtgattggtcgagggatgagcgaccgttaggggcggggcgagtgacgttttgatgacgtggttgcgaggaggagccagtttgcaagttctcgtgggaaaagtgacgtcaaacgaggtgtggtttgaacacggaaatactcaattttcccgcgctctctgacaggaaatgaggtgtttctgggcggatgcaagtgaaaacgggccattttcgcgcgaaaactgaatgaggaagtgaaaatctgagtaatttcgcgtttatggcagggaggagtatttgccgagggccgagtagactttgaccgattacgtgggggtttcgattaccgtgtttttcacctaaatttccgcgtacggtgtcaaagtccggtgtttttacgtaggtgtcagctgatcgccagggtatttaaacctgcgctctccagtcaagaggccactcttgagtgccagcgagaagagttttctcctccgcgccgcgagtcagatctacactttgaaagtagggataacagggtaatgacattgattattgactagttgttaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggaacgccatccacgctgttttgacctccatagaagacagcgatcgcgccaccatggccgggatgttccaggcactgtccgaaggctgcacaccctatgatattaaccagatgctgaatgtcctgggagaccaccaggtctctggcctggagcagctggagagcatcatcaacttcgagaagctgaccgagtggacaagctccaatgtgatgcctatcctgtccccactgaccaagggcatcctgggcttcgtgtttaccctgacagtgccttctgagcggggcctgtcttgcatcagcgaggcagacgcaaccacaccagagtccgccaatctgggcgaggagatcctgtctcagctgtacctgtggccccgggtgacatatcactccccttcttacgcctatcaccagttcgagcggagagccaagtacaagagacacttcccaggctttggccagtctctgctgttcggctaccccgtgtacgtgttcggcgattgcgtgcagggcgactgggatgccatccggtttagatactgcgcaccacctggatatgcactgctgaggtgtaacgacaccaattattccgccctgctggcagtgggcgccctggagggccctcgcaatcaggattggctgggcgtgccaaggcagctggtgacacgcatgcaggccatccagaacgcaggcctgtgcaccctggtggcaatgctggaggagacaatcttctggctgcaggcctttctgatggccctgaccgacagcggccccaagacaaacatcatcgtggattcccagtacgtgatgggcatctccaagccttctttccaggagtttgtggactgggagaacgtgagcccagagctgaattccaccgatcagccattctggcaggcaggaatcctggcaaggaacctggtgcctatggtggccacagtgcagggccagaatctgaagtaccagggccagagcctggtcatcagcgcctccatcatcgtgtttaacctgctggagctggagggcgactatcgggacgatggcaacgtgtgggtgcacaccccactgagccccagaacactgaacgcctgggtgaaggccgtggaggagaagaagggcatcccagtgcacctggagctggcctccatgaccaatatggagctgatgtctagcatcgtgcaccagcaggtgaggacatacggacccgtgttcatgtgcctgggaggcctgctgaccatggtggcaggagccgtgtggctgacagtgcgggtgctggagctgttcagagccgcccagctggccaacgatgtggtgctgcagatcatggagctgtgcggagcagcctttcgccaggtgtgccacaccacagtgccatggcccaatgcctccctgacccccaagtggaacaatgagacaacacagcctcagatcgccaactgtagcgtgtacgacttcttcgtgtggctgcactactatagcgtgagggataccctgtggccccgcgtgacataccacatgaataagtacgcctatcacatgctggagaggcgcgccaagtataagagaggccctggcccaggcgcaaagtttgtggcagcatggaccctgaaggccgccgccggccccggccccggccagtatatcaaggctaacagtaagttcattggaatcacagagctgggacccggacctggataatgagtttaaactcccatttaaatgtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataactataacggtcctaaggtagcgagtgagtagtgttctggggcgggggaggacctgcatgagggccagaataactgaaatctgtgcttttctgtgtgttgcagcagcatgagcggaagcggctcctttgagggaggggtattcagcccttatctgacggggcgtctcccctcctgggcgggagtgcgtcagaatgtgatgggatccacggtggacggccggcccgtgcagcccgcgaactcttcaaccctgacctatgcaaccctgagctcttcgtcgttggacgcagctgccgccgcagctgctgcatctgccgccagcgccgtgcgcggaatggccatgggcgccggctactacggcactctggtggccaactcgagttccaccaataatcccgccagcctgaacgaggagaagctgttgctgctgatggcccagctcgaggccttgacccagcgcctgggcgagctgacccagcaggtggctcagctgcaggagcagacgcgggccgcggttgccacggtgaaatccaaataaaaaatgaatcaataaataaacggagacggttgttgattttaacacagagtctgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcatggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcctttgtgcccgcccaggttttccatgcactcatccatgatgatggcgatgggcccgtgggcggcggcctgggcaaagacgtttcgggggtcggacacatcatagttgtggtcctgggtgaggtcatcataggccattttaatgaatttggggcggagggtgccggactgggggacaaaggtaccctcgatcccgggggcgtagttcccctcacagatctgcatctcccaggctttgagctcggagggggggatcatgtccacctgcggggcgataaagaacacggtttccggggcgggggagatgagctgggccgaaagcaagttccggagcagctgggacttgccgcagccggtggggccgtagatgaccccgatgaccggctgcaggtggtagttgagggagagacagctgccgtcctcccggaggaggggggccacctcgttcatcatctcgcgcacgtgcatgttctcgcgcaccagttccgccaggaggcgctctccccccagggataggagctcctggagcgaggcgaagtttttcagcggcttgagtccgtcggccatgggcattttggagagggtttgttgcaagagttccaggcggtcccagagctcggtgatgtgctctacggcatctcgatccagcagacctcctcgtttcgcgggttgggacggctgcgggagtagggcaccagacgatgggcgtccagcgcagccagggtccggtccttccagggtcgcagcgtccgcgtcagggtggtctccgtcacggtgaaggggtgcgcgccgggctgggcgcttgcgagggtgcgcttcaggctcatccggctggtcgaaaaccgctcccgatcggcgccctgcgcgtcggccaggtagcaattgaccatgagttcgtagttgagcgcctcggccgcgtggcctttggcgcggagcttacctttggaagtctgcccgcaggcgggacagaggagggacttgagggcgtagagcttgggggcgaggaagacggactcgggggcgtaggcgtccgcgccgcagtgggcgcagacggtctcgcactccacgagccaggtgaggtcgggctggtcggggtcaaaaaccagtttcccgccgttctttttgatgcgtttcttacctttggtctccatgagctcgtgtccccgctgggtgacaaagaggctgtccgtgtccccgtagaccgactttatgggccggtcctcgagcggtgtgccgcggtcctcctcgtagaggaaccccgcccactccgagacgaaagcccgggtccaggccagcacgaaggaggccacgtgggacgggtagcggtcgttgtccaccagcgggtccaccttttccagggtatgcaaacacatgtccccctcgtccacatccaggaaggtgattggcttgtaagtgtaggccacgtgaccgggggtcccggccgggggggtataaaagggtgcgggtccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccggcggagatgcctttcaagagcccctcgtccatctggtcagaaaagacgatctttttgttgtcgagcttggtggcgaaggagccgtagagggcgttggagaggagcttggcgatggagcgcatggtctggtttttttccttgtcggcgcgctccttggcggcgatgttgagctgcacgtactcgcgcgccacgcacttccattcggggaagacggtggtcagctcgtcgggcacgattctgacctgccagccccgattatgcagggtgatgaggtccacactggtggccacctcgccgcgcaggggctcattagtccagcagaggcgtccgcccttgcgcgagcagaaggggggcagggggtccagcatgacctcgtcgggggggtcggcatcgatggtgaagatgccgggcaggaggtcggggtcaaagtagctgatggaagtggccagatcgtccagggcagcttgccattcgcgcacggccagcgcgctctcgtagggactgaggggcgtgccccagggcatgggatgggtaagcgcggaggcgtacatgccgcagatgtcgtagacgtagaggggctcctcgaggatgccgatgtaggtggggtagcagcgccccccgcggatgctggcgcgcacgtagtcatacagctcgtgcgagggggcgaggagccccgggcccaggttggtgcgactgggcttttcggcgcggtagacgatctggcggaaaatggcatgcgagttggaggagatggtgggcctttggaagatgttgaagtgggcgtggggcagtccgaccgagtcgcggatgaagtgggcgtaggagtcttgcagcttggcgacgagctcggcggtgactaggacgtccagagcgcagtagtcgagggtctcctggatgatgtcatacttgagctgtcccttttgtttccacagctcgcggttgagaaggaactcttcgcggtccttccagtactcttcgagggggaacccgtcctgatctgcacggtaagagcctagcatgtagaactggttgacggccttgtaggcgcagcagcccttctccacggggagggcgtaggcctgggcggccttgcgcagggaggtgtgcgtgagggcgaaagtgtccctgaccatgaccttgaggaactggtgcttgaagtcgatatcgtcgcagcccccctgctcccagagctggaagtccgtgcgcttcttgtaggcggggttgggcaaagcgaaagtaacatcgttgaagaggatcttgcccgcgcggggcataaagttgcgagtgatgcggaaaggttggggcacctcggcccggttgttgatgacctgggcggcgagcacgatctcgtcgaagccgttgatgttgtggcccacgatgtagagttccacgaatcgcggacggcccttgacgtggggcagtttcttgagctcctcgtaggtgagctcgtcggggtcgctgagcccgtgctgctcgagcgcccagtcggcgagatgggggttggcgcggaggaaggaagtccagagatccacggccagggcggtttgcagacggtcccggtactgacggaactgctgcccgacggccattttttcgggggtgacgcagtagaaggtgcgggggtccccgtgccagcgatcccatttgagctggagggcgagatcgagggcgagctcgacgagccggtcgtccccggagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccattggtggcgacgtcgatggcttgcagggtcccgtgcccctggggtgtgaccaccgtcccccgtttcttcttgggcggctggggcgacgggggcggtgcctcttccatggttagaagcggcggcgaggacgcgcgccgggcggcaggggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcttgaaggtctccgcggccggcgcgctccacggtggccgcgaggtcgttggagatgcggcccatgagctgcgagaaggcgttcatgcccgcctcgttccagacgcggctgtagaccacgacgccctcgggatcgcgggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaaacgttccatggcctcgtaaaagtccacggcgaagttgaaaaactgggagttgcgcgccgagacggtcaactcctcctccagaagacggatgagctcggcgatggtggcgcgcacctcgcgctcgaaggcccccgggagttcctccacttcctcttcttcctcctccactaacatctcttctacttcctcctcaggcggcagtggtggcgggggagggggcctgcgtcgccggcggcgcacgggcagacggtcgatgaagcgctcgatggtctcgccgcgccggcgtcgcatggtctcggtgacggcgcgcccgtcctcgcggggccgcagcgtgaagacgccgccgcgcatctccaggtggccgggggggtccccgttgggcagggagagggcgctgacgatgcatcttatcaattgccccgtagggactccgcgcaaggacctgagcgtctcgagatccacgggatctgaaaaccgctgaacgaaggcttcgagccagtcgcagtcgcaaggtaggctgagcacggtttcttctggcgggtcatgttggttgggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccaggtccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcttgctggatctgggtgagggtggtctggaagtcatcaaagtcgacgaagcggtggtaggctccggtgttgatggtgtaggagcagttggccatgacggaccagttgacggtctggtggcccggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtattggcactcccgtctcgacccaagcctgcaccaaccctccaggatacggaggcgggtcgttttgcaacttttttttggaggccggatgagactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagaccccatagccagccgacttctccagttacggagcgagcccctcttttgttttgtttgtttttgccagatgcatcccgtactgcggcagatgcgcccccaccaccctccaccgcaacaacagccccctccacagccggcgcttctgcccccgccccagcagcaacttccagccacgaccgccgcggccgccgtgagcggggctggacagagttatgatcaccagctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaagcgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagtttgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgtgcggtgagcgccagcaggcggcgcgagctgagcgaccaggagctgatgcatagtctgcagcgggccctgaccggggccgggaccgagggggagagctactttgacatgggcgcggacctgcactggcagcccagccgccgggccttggaggcggcggcaggaccctacgtagaagaggtggacgatgaggtggacgaggagggcgagtacctggaagactgatggcgcgaccgtatttttgctagatgcaacaacaacagccacctcctgatcccgcgatgcgggcggcgctgcagagccagccgtccggcattaactcctcggacgattggacccaggccatgcaacgcatcatggcgctgacgacccgcaaccccgaagcctttagacagcagccccaggccaaccggctctcggccatcctggaggccgtggtgccctcgcgctccaaccccacgcacgagaaggtcctggccatcgtgaacgcgctggtggagaacaaggccatccgcggcgacgaggccggcctggtgtacaacgcgctgctggagcgcgtggcccgctacaacagcaccaacgtgcagaccaacctggaccgcatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggctttcaagaacttgcagggcctgtggggcgtgcaggccccggtcggggaccgcgcgacggtgtcgagcctgctgacgccgaactcgcgcctgctgctgctgctggtggcccccttcacggacagcggcagcatcaaccgcaactcgtacctgggctacctgattaacctgtaccgcgaggccatcggccaggcgcacgtggacgagcagacctaccaggagatcacccacgtgagccgcgccctgggccaggacgacccgggcaacctggaagccaccctgaactttttgctgaccaaccggtcgcagaagatcccgccccagtacgcgctcagcaccgaggaggagcgcatcctgcgttacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctgaatccccactggctcccgccgccggggttctacacgggcgagtacgacatgcccgaccccaatgacgggttcctgtgggacgatgtggacagcagcgtgttctccccccgaccgggtgctaacgagcgccccttgtggaagaaggaaggcagcgaccgacgcccgtcctcggcgctgtccggccgcgagggtgctgccgcggcggtgcccgaggccgccagtcctttcccgagcttgcccttctcgctgaacagtatccgcagcagcgagctgggcaggatcacgcgcccgcgcttgctgggcgaagaggagtacttgaatgactcgctgttgagacccgagcgggagaagaacttccccaataacgggatagaaagcctggtggacaagatgagccgctggaagacgtatgcgcaggagcacagggacgatccccgggcgtcgcagggggccacgagccggggcagcgccgcccgtaaacgccggtggcacgacaggcagcggggacagatgtgggacgatgaggactccgccgacgacagcagcgtgttggacttgggtgggagtggtaacccgttcgctcacctgcgcccccgtatcgggcgcatgatgtaagagaaaccgaaaataaatgatactcaccaaggccatggcgaccagcgtgcgttcgtttcttctctgttgttgttgtatctagtatgatgaggcgtgcgtacccggagggtcctcctccctcgtacgagagcgtgatgcagcaggcgatggcggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggccagctgaaaaccatcatgcacaccaacatgcccaacgtgaacgagttcatgtacagcaacaagttcaaggcgcgggtgatggtctcccgcaagacccccaatggggtgacagtgacagaggattatgatggtagtcaggatgagctgaagtatgaatgggtggaatttgagctgcccgaaggcaacttctcggtgaccatgaccatcgacctgatgaacaacgccatcatcgacaattacttggcggtggggcggcagaacggggtgctggagagcgacatcggcgtgaagttcgacactaggaacttcaggctgggctgggaccccgtgaccgagctggtcatgcccggggtgtacaccaacgaggctttccatcccgatattgtcttgctgcccggctgcggggtggacttcaccgagagccgcctcagcaacctgctgggcattcgcaagaggcagcccttccaggaaggcttccagatcatgtacgaggatctggaggggggcaacatccccgcgctcctggatgtcgacgcctatgagaaaagcaaggaggatgcagcagctgaagcaactgcagccgtagctaccgcctctaccgaggtcaggggcgataattttgcaagcgccgcagcagtggcagcggccgaggcggctgaaaccgaaagtaagatagtcattcagccggtggagaaggatagcaagaacaggagctacaacgtactaccggacaagataaacaccgcctaccgcagctggtacctagcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgcttacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcggtcgcgcaccaccgtcgacgacgtgatcgaccaggtggtggccgacgcgcgcaactacacccccgccgccgcgcccgtctccaccgtggacgccgtcatcgacagcgtggtggccgacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcttcaggcgccagcgccggcaggacccggagacgcgcggccacggcggcggcagcggccatcgccagcatgtcccgcccgcggcgagggaacgtgtactgggtgcgcgacgccgccaccggtgtgcgcgtgcccgtgcgcacccgcccccctcgcacttgaagatgttcacttcgcgatgttgatgtgtcccagcggcgaggaggatgtccaagcgcaaattcaaggaagagatgctccaggtcatcgcgcctgagatctacggccctgcggtggtgaaggaggaaagaaagccccgcaaaatcaagcgggtcaaaaaggacaaaaaggaagaagaaagtgatgtggacggattggtggagtttgtgcgcgagttcgccccccggcggcgcgtgcagtggcgcgggcggaaggtgcaaccggtgctgagacccggcaccaccgtggtcttcacgcccggcgagcgctccggcaccgcttccaagcgctcctacgacgaggtgtacggggatgatgatattctggagcaggcggccgagcgcctgggcgagtttgcttacggcaagcgcagccgttccgcaccgaaggaagaggcggtgtccatcccgctggaccacggcaaccccacgccgagcctcaagcccgtgaccttgcagcaggtgctgccgaccgcggcgccgcgccgggggttcaagcgcgagggcgaggatctgtaccccaccatgcagctgatggtgcccaagcgccagaagctggaagacgtgctggagaccatgaaggtggacccggacgtgcagcccgaggtcaaggtgcggcccatcaagcaggtggccccgggcctgggcgtgcagaccgtggacatcaagattcccacggagcccatggaaacgcagaccgagcccatgatcaagcccagcaccagcaccatggaggtgcagacggatccctggatgccatcggctcctagtcgaagaccccggcgcaagtacggcgcggccagcctgctgatgcccaactacgcgctgcatccttccatcatccccacgccgggctaccgcggcacgcgcttctaccgcggtcataccagcagccgccgccgcaagaccaccactcgccgccgccgtcgccgcaccgccgctgcaaccacccctgccgccctggtgcggagagtgtaccgccgcggccgcgcacctctgaccctgccgcgcgcgcgctaccacccgagcatcgccatttaaactttcgcctgctttgcagatcaatggccctcacatgccgccttcgcgttcccattacgggctaccgaggaagaaaaccgcgccgtagaaggctggcggggaacgggatgcgtcgccaccaccaccggcggcggcgcgccatcagcaagcggttggggggaggcttcctgcccgcgctgatccccatcatcgccgcggcgatcggggcgatccccggcattgcttccgtggcggtgcaggcctctcagcgccactgagacacacttggaaacatcttgtaataaaccaatggactctgacgctcctggtcctgtgatgtgttttcgtagacagatggaagacatcaatttttcgtccctggctccgcgacacggcacgcggccgttcatgggcacctggagcgacatcggcaccagccaactgaacgggggcgccttcaattggagcagtctctggagcgggcttaagaatttcgggtccacgcttaaaacctatggcagcaaggcgtggaacagcaccacagggcaggcgctgagggataagctgaaagagcagaacttccagcagaaggtggtcgatgggctcgcctcgggcatcaacggggtggtggacctggccaaccaggccgtgcagcggcagatcaacagccgcctggacccggtgccgcccgccggctccgtggagatgccgcaggtggaggaggagctgcctcccctggacaagcggggcgagaagcgaccccgccccgatgcggaggagacgctgctgacgcacacggacgagccgcccccgtacgaggaggcggtgaaactgggtctgcccaccacgcggcccatcgcgcccctggccaccggggtgctgaaacccgaaaagcccgcgaccctggacttgcctcctccccagccttcccgcccctctacagtggctaagcccctgccgccggtggccgtggcccgcgcgcgacccgggggcaccgcccgccctcatgcgaactggcagagcactctgaacagcatcgtgggtctgggagtgcagagtgtgaagcgccgccgctgctattaaacctaccgtagcgcttaacttgcttgtctgtgtgtgtatgtattatgtcgccgccgccgctgtccaccagaaggaggagtgaagaggcgcgtcgccgagttgcaagatggccaccccatcgatgctgccccagtgggcgtacatgcacatcgccggacaggacgcttcggagtacctgagtccgggtctggtgcagtttgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccctagcttcaaaccctactccggcaccgcctacaacagtctggcccccaagggagcacccaacacttgtcagtggacatataaagccgatggtgaaactgccacagaaaaaacctatacatatggaaatgcacccgtgcagggcattaacatcacaaaagatggtattcaacttggaactgacaccgatgatcagccaatctacgcagataaaacctatcagcctgaacctcaagtgggtgatgctgaatggcatgacatcactggtactgatgaaaagtatggaggcagagctcttaagcctgataccaaaatgaagccttgttatggttcttttgccaagcctactaataaagaaggaggtcaggcaaatgtgaaaacaggaacaggcactactaaagaatatgacatagacatggctttctttgacaacagaagtgcggctgctgctggcctagctccagaaattgttttgtatactgaaaatgtggatttggaaactccagatacccatattgtatacaaagcaggcacagatgacagcagctcttctattaatttgggtcagcaagccatgcccaacagacctaactacattggtttcagagacaactttatcgggctcatgtactacaacagcactggcaatatgggggtgctggccggtcaggcttctcagctgaatgctgtggttgacttgcaagacagaaacaccgagctgtcctaccagctcttgcttgactctctgggtgacagaacccggtatttcagtatgtggaatcaggcggtggacagctatgatcctgatgtgcgcattattgaaaatcatggtgtggaggatgaacttcccaactattgtttccctctggatgctgttggcagaacagatacttatcagggaattaaggctaatggaactgatcaaaccacatggaccaaagatgacagtgtcaatgatgctaatgagataggcaagggtaatccattcgccatggaaatcaacatccaagccaacctgtggaggaacttcctctacgccaacgtggccctgtacctgcccgactcttacaagtacacgccggccaatgttaccctgcccaccaacaccaacacctacgattacatgaacggccgggtggtggcgccctcgctggtggactcctacatcaacatcggggcgcgctggtcgctggatcccatggacaacgtgaaccccttcaaccaccaccgcaatgcggggctgcgctaccgctccatgctcctgggcaacgggcgctacgtgcccttccacatccaggtgccccagaaatttttcgccatcaagagcctcctgctcctgcccgggtcctacacctacgagtggaacttccgcaaggacgtcaacatgatcctgcagagctccctcggcaacgacctgcgcacggacggggcctccatctccttcaccagcatcaacctctacgccaccttcttccccatggcgcacaacacggcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggctggtccttcacgcgtctcaagaccaaggagacgccctcgctgggctccgggttcgacccctacttcgtctactcgggctccatcccctacctcgacggcaccttctacctcaaccacaccttcaagaaggtctccatcaccttcgactcctccgtcagctggcccggcaacgaccggctcctgacgcccaacgagttcgaaatcaagcgcaccgtcgacggcgagggctacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtggtggacgaggtcaactacaaggactaccaggccgtcaccctggcctaccagcacaacaactcgggcttcgtcggctacctcgcgcccaccatgcgccagggccagccctaccccgccaactacccctacccgctcatcggcaagagcgccgtcaccagcgtcacccagaaaaagttcctctgcgacagggtcatgtggcgcatccccttctccagcaacttcatgtccatgggcgcgctcaccgacctcggccagaacatgctctatgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacccccttctcggccggtaacgccaccacctaagctcttgcttcttgcaagccatggccgcgggctccggcgagcaggagctcagggccatcatccgcgacctgggctgcgggccctacttcctgggcaccttcgataagcgcttcccgggattcatggccccgcacaagctggcctgcgccatcgtcaacacggccggccgcgagaccgggggcgagcactggctggccttcgcctggaacccgcgctcgaacacctgctacctcttcgaccccttcgggttctcggacgagcgcctcaagcagatctaccagttcgagtacgagggcctgctgcgccgcagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaactcccactccgcctactttcgctcccaccgcgcgcgcatcgagaaggccaccgccttcgaccgcatgaatcaagacatgtaaaccgtgtgtgtatgttaaatgtctttaataaacagcactttcatgttacacatgcatctgagatgatttatttagaaatcgaaagggttctgccgggtctcggcatggcccgcgggcagggacacgttgcggaactggtacttggccagccacttgaactcggggatcagcagtttgggcagcggggtgtcggggaaggagtcggtccacagcttccgcgtcagttgcagggcgcccagcaggtcgggcgcggagatcttgaaatcgcagttgggacccgcgttctgcgcgcgggagttgcggtacacggggttgcagcactggaacaccatcagggccgggtgcttcacgctcgccagcaccgtcgcgtcggtgatgctctccacgtcgaggtcctcggcgttggccatcccgaagggggtcatcttgcaggtctgccttcccatggtgggcacgcacccgggcttgtggttgcaatcgcagtgcagggggatcagcatcatctgggcctggtcggcgttcatccccgggtacatggccttcatgaaagcctccaattgcctgaacgcctgctgggccttggctccctcggtgaagaagaccccgcaggacttgctagagaactggttggtggcgcacccggcgtcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcatgtgctccttctggatcatggtggtcccgtgcaggcaccgcagcttgccctcggcctcggtgcacccgtgcagccacagcgcgcacccggtgcactcccagttcttgtgggcgatctgggaatgcgcgtgcacgaagccctgcaggaagcggcccatcatggtggtcagggtcttgttgctagtgaaggtcagcggaatgccgcggtgctcctcgttgatgtacaggtggcagatgcggcggtacacctcgccctgctcgggcatcagctggaagttggctttcaggtcggtctccacgcggtagcggtccatcagcatagtcatgatttccatacccttctcccaggccgagacgatgggcaggctcatagggttcttcaccatcatcttagcgctagcagccgcggccagggggtcgctctcgtccagggtctcaaagctccgcttgccgtccttctcggtgatccgcaccggggggtagctgaagcccacggccgccagctcctcctcggcctgtctttcgtcctcgctgtcctggctgacgtcctgcaggaccacatgcttggtcttgcggggtttcttcttgggcggcagcggcggcggagatgttggagatggcgagggggagcgcgagttctcgctcaccactactatctcttcctcttcttggtccgaggccacgcggcggtaggtatgtctcttcgggggcagaggcggaggcgacgggctctcgccgccgcgacttggcggatggctggcagagccccttccgcgttcgggggtgcgctcccggcggcgctctgactgacttcctccgcggccggccattgtgttctcctagggaggaacaacaagcatggagactcagccatcgccaacctcgccatctgcccccaccgccgacgagaagcagcagcagcagaatgaaagcttaaccgccccgccgcccagccccgccacctccgacgcggccgtcccagacatgcaagagatggaggaatccatcgagattgacctgggctatgtgacgcccgcggagcacgaggaggagctggcagtgcgcttttcacaagaagagatacaccaagaacagccagagcaggaagcagagaatgagcagagtcaggctgggctcgagcatgacggcgactacctccacctgagcgggggggaggacgcgctcatcaagcatctggcccggcaggccaccatcgtcaaggatgcgctgctcgaccgcaccgaggtgcccctcagcgtggaggagctcagccgcgcctacgagttgaacctcttctcgccgcgcgtgccccccaagcgccagcccaatggcacctgcgagcccaacccgcgcctcaacttctacccggtcttcgcggtgcccgaggccctggccacctaccacatctttttcaagaaccaaaagatccccgtctcctgccgcgccaaccgcacccgcgccgacgcccttttcaacctgggtcccggcgcccgcctacctgatatcgcctccttggaagaggttcccaagatcttcgagggtctgggcagcgacgagactcgggccgcgaacgctctgcaaggagaaggaggagagcatgagcaccacagcgccctggtcgagttggaaggcgacaacgcgcggctggcggtgctcaaacgcacggtcgagctgacccatttcgcctacccggctctgaacctgccccccaaagtcatgagcgcggtcatggaccaggtgctcatcaagcgcgcgtcgcccatctccgaggacgagggcatgcaagactccgaggagggcaagcccgtggtcagcgacgagcagctggcccggtggctgggtcctaatgctagtccccagagtttggaagagcggcgcaaactcatgatggccgtggtcctggtgaccgtggagctggagtgcctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcttgcacgagaaccgcctggggcagaacgtgctgcacaccaccctgcgcggggaggcccggcgcgactacatccgcgactgcgtctacctctacctctgccacacctggcagacgggcatgggcgtgtggcagcagtgtctggaggagcagaacctgaaagagctctgcaagctcctgcagaagaacctcaagggtctgtggaccgggttcgacgagcgcaccaccgcctcggacctggccgacctcattttccccgagcgcctcaggctgacgctgcgcaacggcctgcccgactttatgagccaaagcatgttgcaaaactttcgctctttcatcctcgaacgctccggaatcctgcccgccacctgctccgcgctgccctcggacttcgtgccgctgaccttccgcgagtgccccccgccgctgtggagccactgctacctgctgcgcctggccaactacctggcctaccactcggacgtgatcgaggacgtcagcggcgagggcctgctcgagtgccactgccgctgcaacctctgcacgccgcaccgctccctggcctgcaacccccagctgctgagcgagacccagatcatcggcaccttcgagttgcaagggcccagcgaaggcgagggttcagccgccaaggggggtctgaaactcaccccggggctgtggacctcggcctacttgcgcaagttcgtgcccgaggactaccatcccttcgagatcaggttctacgaggaccaatcccatccgcccaaggccgagctgtcggcctgcgtcatcacccagggggcgatcctggcccaattgcaagccatccagaaatcccgccaagaattcttgctgaaaaagggccgcggggtctacctcgacccccagaccggtgaggagctcaaccccggcttcccccaggatgccccgaggaaacaagaagctgaaagtggagctgccgcccgtggaggatttggaggaagactgggagaacagcagtcaggcagaggaggaggagatggaggaagactgggacagcactcaggcagaggaggacagcctgcaagacagtctggaggaagacgaggaggaggcagaggaggaggtggaagaagcagccgccgccagaccgtcgtcctcggcgggggagaaagcaagcagcacggataccatctccgctccgggtcggggtcccgctcgaccacacagtagatgggacgagaccggacgattcccgaaccccaccacccagaccggtaagaaggagcggcagggatacaagtcctggcgggggcacaaaaacgccatcgtctcctgcttgcaggcctgcgggggcaacatctccttcacccggcgctacctgctcttccaccgcggggtgaactttccccgcaacatcttgcattactaccgtcacctccacagcccctactacttccaagaagaggcagcagcagcagaaaaagaccagcagaaaaccagcagctagaaaatccacagcggcggcagcaggtggactgaggatcgcggcgaacgagccggcgcaaacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccagtcgcagaaaaaggcgggaattacgtcacctgtgcccttcgccctagccgcctccacccatcatcatgagcaaagagattcccacgccttacatgtggagctaccagccccagatgggcctggccgccggtgccgcccaggactactccacccgcatgaattggctcagcgccgggcccgcgatgatctcacgggtgaatgacatccgcgcccaccgaaaccagatactcctagaacagtcagcgctcaccgccacgccccgcaatcacctcaatccgcgtaattggcccgccgccctggtgtaccaggaaattccccagcccacgaccgtactacttccgcgagacgcccaggccgaagtccagctgactaactcaggtgtccagctggcgggcggcgccaccctgtgtcgtcaccgccccgctcagggtataaagcggctggtgatccggggcagaggcacacagctcaacgacgaggtggtgagctcttcgctgggtctgcgacctgacggagtcttccaactcgccggatcggggagatcttccttcacgcctcgtcaggccgtcctgactttggagagttcgtcctcgcagccccgctcgggtggcatcggcactctccagttcgtggaggagttcactccctcggtctacttcaaccccttctccggctcccccggccactacccggacgagttcatcccgaacttcgacgccatcagcgagtcggtggacggctacgattgaatgtcccatggtggcgcagctgacctagctcggcttcgacacctggaccactgccgccgcttccgctgcttcgctcgggatctcgccgagtttgcctactttgagctgcccgaggagcaccctcagggcccggcccacggagtgcggatcgtcgtcgaagggggcctcgactcccacctgcttcggatcttcagccagcgtccgatcctggtcgagcgcgagcaaggacagacccttctgactctgtactgcatctgcaaccaccccggcctgcatgaaagtctttgttgtctgctgtgtactgagtataataaaagctgagatcagcgactactccggacttccgtgtgttcctgaatccatcaaccagtctttgttcttcaccgggaacgagaccgagctccagctccagtgtaagccccacaagaagtacctcacctggctgttccagggctccccgatcgccgttgtcaaccactgcgacaacgacggagtcctgctgagcggccctgccaaccttactttttccacccgcagaagcaagctccagctcttccaacccttcctccccgggacctatcagtgcgtctcgggaccctgccatcacaccttccacctgatcccgaataccacagcgtcgctccccgctactaacaaccaaactaacctccaccaacgccaccgtcgcgacggccacaatacatgcccatattagactatgaggccgagccacagcgacccatgctccccgctattagttacttcaatctaaccggcggagatgactgacccactggccaacaacaacgtcaacgaccttctcctggacatggacggccgcgcctcggagcagcgactcgcccaacttcgcattcgccagcagcaggagagagccgtcaaggagctgcaggatgcggtggccatccaccagtgcaagagaggcatcttctgcctggtgaaacaggccaagatctcctacgaggtcactccaaacgaccatcgcctctcctacgagctcctgcagcagcgccagaagttcacctgcctggtcggagtcaaccccatcgtcatcacccagcagtctggcgataccaaggggtgcatccactgctcctgcgactcccccgactgcgtccacactctgatcaagaccctctgcggcctccgcgacctcctccccatgaactaatcacccccttatccagtgaaataaagatcatattgatgatgattttacagaaataaaaaataatcatttgatttgaaataaagatacaatcatattgatgatttgagtttaacaaaaaaataaagaatcacttacttgaaatctgataccaggtctctgtccatgttttctgccaacaccacttcactcccctcttcccagctctggtactgcaggccccggcgggctgcaaacttcctccacacgctgaaggggatgtcaaattcctcctgtccctcaatcttcattttatcttctatcagatgtccaaaaagcgcgtccgggtggatgatgacttcgaccccgtctacccctacgatgcagacaacgcaccgaccgtgcccttcatcaacccccccttcgtctcttcagatggattccaagagaagcccctgggggtgttgtccctgcgactggccgaccccgtcaccaccaagaacggggaaatcaccctcaagctgggagagggggtggacctcgattcctcgggaaaactcatctccaacacggccaccaaggccgccgcccctctcagtttttccaacaacaccatttcccttaacatggatcaccccttttacactaaagatggaaaattatccttacaagtttctccaccattaaatatactgagaacaagcattctaaacacactagctttaggttttggatcaggtttaggactccgtggctctgccttggcagtacagttagtctctccacttacatttgatactgatggaaacataaagcttaccttagacagaggtttgcatgttacaacaggagatgcaattgaaagcaacataagctgggctaaaggtttaaaatttgaagatggagccatagcaaccaacattggaaatgggttagagtttggaagcagtagtacagaaacaggtgttgatgatgcttacccaatccaagttaaacttggatctggccttagctttgacagtacaggagccataatggctggtaacaaagaagacgataaactcactttgtggacaacacctgatccatcaccaaactgtcaaatactcgcagaaaatgatgcaaaactaacactttgcttgactaaatgtggtagtcaaatactggccactgtgtcagtcttagttgtaggaagtggaaacctaaaccccattactggcaccgtaagcagtgctcaggtgtttctacgttttgatgcaaacggtgttcttttaacagaacattctacactaaaaaaatactgggggtataggcagggagatagcatagatggcactccatataccaatgctgtaggattcatgcccaatttaaaagcttatccaaagtcacaaagttctactactaaaaataatatagtagggcaagtatacatgaatggagatgtttcaaaacctatgcttctcactataaccctcaatggtactgatgacagcaacagtacatattcaatgtcattttcatacacctggactaatggaagctatgttggagcaacatttggggctaactcttataccttctcatacatcgcccaagaatgaacactgtatcccaccctgcatgccaacccttcccaccccactctgtggaacaaactctgaaacacaaaataaaataaagttcaagtgttttattgattcaacagttttacaggattcgagcagttatttttcctccaccctcccaggacatggaatacaccaccctctccccccgcacagccttgaacatctgaatgccattggtgatggacatgcttttggtctccacgttccacacagtttcagagcgagccagtctcgggtcggtcagggagatgaaaccctccgggcactcccgcatctgcacctcacagctcaacagctgaggattgtcctcggtggtcgggatcacggttatctggaagaagcagaagagcggcggtgggaatcatagtccgcgaacgggatcggccggtggtgtcgcatcaggccccgcagcagtcgctgccgccgccgctccgtcaagctgctgctcagggggtccgggtccagggactccctcagcatgatgcccacggccctcagcatcagtcgtctggtgcggcgggcgcagcagcgcatgcggatctcgctcaggtcgctgcagtacgtgcaacacagaaccaccaggttgttcaacagtccatagttcaacacgctccagccgaaactcatcgcgggaaggatgctacccacgtggccgtcgtaccagatcctcaggtaaatcaagtggtgccccctccagaacacgctgcccacgtacatgatctccttgggcatgtggcggttcaccacctcccggtaccacatcaccctctggttgaacatgcagccccggatgatcctgcggaaccacagggccagcaccgccccgcccgccatgcagcgaagagaccccgggtcccggcaatggcaatggaggacccaccgctcgtacccgtggatcatctgggagctgaacaagtctatgttggcacagcacaggcatatgctcatgcatctcttcagcactctcaactcctcgggggtcaaaaccatatcccagggcacggggaactcttgcaggacagcgaaccccgcagaacagggcaatcctcgcacagaacttacattgtgcatggacagggtatcgcaatcaggcagcaccgggtgatcctccaccagagaagcgcgggtctcggtctcctcacagcgtggtaagggggccggccgatacgggtgatggcgggacgcggctgatcgtgttcgcgaccgtgtcatgatgcagttgctttcggacattttcgtacttgctgtagcagaacctggtccgggcgctgcacaccgatcgccggcggcggtctcggcgcttggaacgctcggtgttgaaattgtaaaacagccactctctcagaccgtgcagcagatctagggcctcaggagtgatgaagatcccatcatgcctgatggctctgatcacatcgaccaccgtggaatgggccagacccagccagatgatgcaattttgttgggtttcggtgacggcgggggagggaagaacaggaagaaccatgattaacttttaatccaaacggtctcggagtacttcaaaatgaagatcgcggagatggcacctctcgcccccgctgtgttggtggaaaataacagccaggtcaaaggtgatacggttctcgagatgttccacggtggcttccagcaaagcctccacgcgcacatccagaaacaagacaatagcgaaagcgggagggttctctaattcctcaatcatcatgttacactcctgcaccatccccagataattttcatttttccagccttgaatgattcgaactagttcgtgaggtaaatccaagccagccatgataaagagctcgcgcagagcgccctccaccggcattcttaagcacaccctcataattccaagatattctgctcctggttcacctgcagcagattgacaagcggaatatcaaaatctctgccgcgatccctgagctcctccctcagcaataactgtaagtactctttcatatcctctccgaaatttttagccataggaccaccaggaataagattagggcaagccacagtacagataaaccgaagtcctccccagtgagcattgccaaatgcaagactgctataagcatgctggctagacccggtgatatcttccagataactggacagaaaatcgcccaggcaatttttaagaaaatcaacaaaagaaaaatcctccaggtggacgtttagagcctcgggaacaacgatgaagtaaatgcaagcggtgcgttccagcatggttagttagctgatctgtagaaaaaacaaaaatgaacattaaaccatgctagcctggcgaacaggtgggtaaatcgttctctccagcaccaggcaggccacggggtctccggcgcgaccctcgtaaaaattgtcgctatgattgaaaaccatcacagagagacgttcccggtggccggcgtgaatgattcgacaagatgaatacacccccggaacattggcgtccgcgagtgaaaaaaagcgcccgaggaagcaataaggcactacaatgctcagtctcaagtccagcaaagcgatgccatgcggatgaagcacaaaattctcaggtgcgtacaaaatgtaattactcccctcctgcacaggcagcaaagcccccgatccctccaggtacacatacaaagcctcagcgtccatagcttaccgagcagcagcacacaacaggcgcaagagtcagagaaaggctgagctctaacctgtccacccgctctctgctcaatatatagcccagatctacactgacgtaaaggccaaagtctaaaaatacccgccaaataatcacacacgcccagcacacgcccagaaaccggtgacacactcaaaaaaatacgcgcacttcctcaaacgcccaaaactgccgtcatttccgggttcccacgctacgtcatcaaaacacgactttcaaattccgtcgaccgttaaaaacgtcacccgccccgcccctaacggtcgcccgtctctcagccaatcagcgccccgcatccccaaattcaaacacctcatttgcatattaacgcgcacaaaaagtttgaggtatattattgatgatgg

SEQ ID NO: 13 - DNA, 30684 bp, synthetic construct
  misc_feature 1..30684: note = Description of Artificial Sequence: Synthetic polynucleotide
  source 1..30684: mol_type = other DNA
ccatcttcaataatatacctcaaactttttgtgcgcgttaatatgcaaatgaggcgtttgaatttggggaggaagggcggtgattggtcgagggatgagcgaccgttaggggcggggcgagtgacgttttgatgacgtggttgcgaggaggagccagtttgcaagttctcgtgggaaaagtgacgtcaaacgaggtgtggtttgaacacggaaatactcaattttcccgcgctctctgacaggaaatgaggtgtttctgggcggatgcaagtgaaaacgggccattttcgcgcgaaaactgaatgaggaagtgaaaatctgagtaatttcgcgtttatggcagggaggagtatttgccgagggccgagtagactttgaccgattacgtgggggtttcgattaccgtgtttttcacctaaatttccgcgtacggtgtcaaagtccggtgtttttacgtaggtgtcagctgatcgccagggtatttaaacctgcgctctccagtcaagaggccactcttgagtgccagcgagaagagttttctcctccgcgccgcgagtcagatctacactttgaaagtagggataacagggtaatgacattgattattgactagttgttaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggaacgccatccacgctgttttgacctccatagaagacagcgatcgcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctttacaagtagtgagtttaaactcccatttaaatgtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaataactataacggtcctaaggtagcgagtgagtagtgttctggggcgggggaggacctgcatgagggccagaataactgaaatctgtgcttttctgtgtgttgcagcagcatgagcggaagcggctcctttgagggaggggtattcagcccttatctgacggggcgtctcccctcctgggcgggagtgcgtcagaatgtgatgggatccacggtggacggccggcccgtgcagcccgcgaactcttcaaccctgacctatgcaaccctgagctcttcgtcgttggacgcagctgccgccgcagctgctgcatctgccgccagcgccgtgcgcggaatggccatgggcgccggctactacggcactctggtggccaactcgagttccaccaataatcccgccagcctgaacgaggagaagctgttgctgctgatggcccagctcgaggccttgacccagcgcctgggcgagctgacccagcaggtggctcagctgcaggagcagacgcgggccgcggttgccacggtgaaatccaaataaaaaatgaatcaataaataaacggagacggttgttgattttaacacagagtctgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcatggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcctttgtgcccgcccaggttttccatgcactcatccatgatgatggcgatgggcccgtgggcggcggcctgggcaaagacgtttcgggggtcggacacatcatagttgtggtcctgggtgaggtcatcataggccattttaatgaatttggggcggagggtgccggactgggggacaaaggtaccctcgatcccgggggcgtagttcccctcacagatctgcatctcccaggctttgagctcggagggggggatcatgtccacctgcggggcgataaagaacacggtttccggggcgggggagatgagctgggccgaaagcaagttccggagcagctgggacttgccgcagccggtggggccgtagatgaccccgatgaccggctgcaggtggtagttgagggagagacagctgccgtcctcccggaggaggggggccacctcgttcatcatctcgcgcacgtgcatgttctcgcgcaccagttccgccaggaggcgctctccccccagggataggagctcctggagcgaggcgaagtttttcagcggcttgagtccgtcggccatgggcattttggagagggtttgttgcaagagttccaggcggtcccagagctcggtgatgtgctctacggcatctcgatccagcagacctcctcgtttcgcgggttgggacggctgcgggagtagggcaccagacgatgggcgtccagcgcagccagggtccggtccttccagggtcgcagcgtccgcgtcagggtggtctccgtcacggtgaaggggtgcgcgccgggctgggcgcttgcgagggtgcgcttcaggctcatccggctggtcgaaaaccgctcccgatcggcgccctgcgcgtcggccaggtagcaattgaccatgagttcgtagttgagcgcctcggccgcgtggcctttggcgcggagcttacctttggaagtctgcccgcaggcgggacagaggagggacttgagggcgtagagcttgggggcgaggaagacggactcgggggcgtaggcgtccgcgccgcagtgggcgcagacggtctcgcactccacgagccaggtgaggtcgggctggtcggggtcaaaaaccagtttcccgccgttctttttgatgcgtttcttacctttggtctccatgagctcgtgtccccgctgggtgacaaagaggctgtccgtgtccccgtagaccgactttatgggccggtcctcgagcggtgtgccgcggtcctcctcgtagaggaaccccgcccactccgagacgaaagcccgggtccaggccagcacgaaggaggccacgtgggacgggtagcggtcgttgtccaccagcgggtccaccttttccagggtatgcaaacacatgtccccctcgtccacatccaggaaggtgattggcttgtaagtgtaggccacgtgaccgggggtcccggccgggggggtataaaagggtgcgggtccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccggcggagatgcctttcaagagcccctcgtccatctggtcagaaaagacgatctttttgttgtcgagcttggtggcgaaggagccgtagagggcgttggagaggagcttggcgatggagcgcatggtctggtttttttccttgtcggcgcgctccttggcggcgatgttgagctgcacgtactcgcgcgccacgcacttccattcggggaagacggtggtcagctcgtcgggcacgattctgacctgccagccccgattatgcagggtgatgaggtccacactggtggccacctcgccgcgcaggggctcattagtccagcagaggcgtccgcccttgcgcgagcagaaggggggcagggggtccagcatgacctcgtcgggggggtcggcatcgatggtgaagatgccgggcaggaggtcggggtcaaagtagctgatggaagtggccagatcgtccagggcagcttgccattcgcgcacggccagcgcgcgctcgtagggactgaggggcgtgccccagggcatgggatgggtaagcgcggaggcgtacatgccgcagatgtcgtagacgtagaggggctcctcgaggatgccgatgtaggtggggtagcagcgccccccgcggatgctggcgcgcacgtagtcatacagctcgtgcgagggggcgaggagccccgggcccaggttggtgcgactgggcttttcggcgcggtagacgatctggcggaaaatggcatgcgagttggaggagatggtgggcctttggaagatgttgaagtgggcgtggggcagtccgaccgagtcgcggatgaagtgggcgtaggagtcttgcagcttggcgacgagctcggcggtgactaggacgtccagagcgcagtagtcgagggtctcctggatgatgtcatacttgagctgtcccttttgtttccacagctcgcggttgagaaggaactcttcgcggtccttccagtactcttcgagggggaacccgtcctgatctgcacggtaagagcctagcatgtagaactggttgacggccttgtaggcgcagcagcccttctccacggggagggcgtaggcctgggcggccttgcgcagggaggtgtgcgtgagggcgaaagtgtccctgaccatgaccttgaggaactggtgcttgaagtcgatatcgtcgcagcccccctgctcccagagctggaagtccgtgcgcttcttgtaggcggggttgggcaaagcgaaagtaacatcgttgaagaggatcttgcccgcgcggggcataaagttgcgagtgatgcggaaaggttggggcacctcggcccggttgttgatgacctgggcggcgagcacgatctcgtcgaagccgttgatgttgtggcccacgatgtagagttccacgaatcgcggacggcccttgacgtggggcagtttcttgagctcctcgtaggtgagctcgtcggggtcgctgagcccgtgctgctcgagcgcccagtcggcgagatgggggttggcgcggaggaaggaagtccagagatccacggccagggcggtttgcagacggtcccggtactgacggaactgctgcccgacggccattttttcgggggtgacgcagtagaaggtgcgggggtccccgtgccagcgatcccatttgagctggagggcgagatcgagggcgagctcgacgagccggtcgtccccggagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccattggtggcgacgtcgatggcttgcagggtcccgtgcccctggggtgtgaccaccgtcccccgtttcttcttgggcggctggggcgacgggggcggtgcctcttccatggttagaagcggcggcgaggacgcgcgccgggcggcaggggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcttgaaggtctccgcggccggcgcgctccacggtggccgcgaggtcgttggagatgcggcccatgagctgcgagaaggcgttcatgcccgcctcgttccagacgcggctgtagaccacgacgccctcgggatcgcgggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaaacgttccatggcctcgtaaaagtccacggcgaagttgaaaaactgggagttgcgcgccgagacggtcaactcctcctccagaagacggatgagctcggcgatggtggcgcgcacctcgcgctcgaaggcccccgggagttcctccacttcctcttcttcctcctccactaacatctcttctacttcctcctcaggcggcagtggtggcgggggagggggcctgcgtcgccggcggcgcacgggcagacggtcgatgaagcgctcgatggtctcgccgcgccggcgtcgcatggtctcggtgacggcgcgcccgtcctcgcggggccgcagcgtgaagacgccgccgcgcatctccaggtggccgggggggtccccgttgggcagggagagggcgctgacgatgcatcttatcaattgccccgtagggactccgcgcaaggacctgagcgtctcgagatccacgggatctgaaaaccgctgaacgaaggcttcgagccagtcgcagtcgcaaggtaggctgagcacggtttcttctggcgggtcatgttggttgggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccaggtccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcttgctggatctgggtgagggtggtctggaagtcatcaaagtcgacgaagcggtggtaggctccggtgttgatggtgtaggagcagttggccatgacggaccagttgacggtctggtggcccggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtattggcactcccgtctcgacccaagcctgcaccaaccctccaggatacggaggcgggtcgttttgcaacttttttttggaggccggatgagactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagaccccatagccagccgacttctccagttacggagcgagcccctcttttgttttgtttgtttttgccagatgcatcccgtactgcggcagatgcgcccccaccaccctccaccgcaacaacagccccctccacagccggcgcttctgcccccgccccagcagcaacttccagccacgaccgccgcggccgccgtgagcggggctggacagagttatgatcaccagctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaagcgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagtttgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgtgcggtgagcgccagcaggcggcgcgagctgagcgaccaggagctgatgcatagtctgcagcgggccctgaccggggccgggaccgagggggagagctactttgacatgggcgcggacctgcactggcagcccagccgccgggccttggaggcggcggcaggaccctacgtagaagaggtggacgatgaggtggacgaggagggcgagtacctggaagactgatggcgcgaccgtatttttgctagatgcaacaacaacagccacctcctgatcccgcgatgcgggcggcgctgcagagccagccgtccggcattaactcctcggacgattggacccaggccatgcaacgcatcatggcgctgacgacccgcaaccccgaagcctttagacagcagccccaggccaaccggctctcggccatcctggaggccgtggtgccctcgcgctccaaccccacgcacgagaaggtcctggccatcgtgaacgcgctggtggagaacaaggccatccgcggcgacgaggccggcctggtgtacaacgcgctgctggagcgcgtggcccgctacaacagcaccaacgtgcagaccaacctggaccgcatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggctttcaagaacttgcagggcctgtggggcgtgcaggccccggtcggggaccgcgcgacggtgtcgagcctgctgacgccgaactcgcgcctgctgctgctgctggtggcccccttcacggacagcggcagcatcaaccgcaactcgtacctgggctacctgattaacctgtaccgcgaggccatcggccaggcgcacgtggacgagcagacctaccaggagatcacccacgtgagccgcgccctgggccaggacgacccgggcaacctggaagccaccctgaactttttgctgaccaaccggtcgcagaagatcccgccccagtacgcgctcagcaccgaggaggagcgcatcctgcgttacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctgaatccccactggctcccgccgccggggttctacacgggcgagtacgacatgcccgaccccaatgacgggttcctgtgggacgatgtggacagcagcgtgttctccccccgaccgggtgctaacgagcgccccttgtggaagaaggaaggcagcgaccgacgcccgtcctcggcgctgtccggccgcgagggtgctgccgcggcggtgcccgaggccgccagtcctttcccgagcttgcccttctcgctgaacagtatccgcagcagcgagctgggcaggatcacgcgcccgcgcttgctgggcgaagaggagtacttgaatgactcgctgttgagacccgagcgggagaagaacttccccaataacgggatagaaagcctggtggacaagatgagccgctggaagacgtatgcgcaggagcacagggacgatccccgggcgtcgcagggggccacgagccggggcagcgccgcccgtaaacgccggtggcacgacaggcagcggggacagatgtgggacgatgaggactccgccgacgacagcagcgtgttggacttgggtgggagtggtaacccgttcgctcacctgcgcccccgtatcgggcgcatgatgtaagagaaaccgaaaataaatgatactcaccaaggccatggcgaccagcgtgcgttcgtttcttctctgttgttgttgtatctagtatgatgaggcgtgcgtacccggagggtcctcctccctcgtacgagagcgtgatgcagcaggcgatggcggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggccagctgaaaaccatcatgcacaccaacatgcccaacgtgaacgagttcatgtacagcaacaagttcaaggcgcgggtgatggtctcccgcaagacccccaatggggtgacagtgacagaggattatgatggtagtcaggatgagctgaagtatgaatgggtggaatttgagctgcccgaaggcaacttctcggtgaccatgaccatcgacctgatgaacaacgccatcatcgacaattacttggcggtggggcggcagaacggggtgctggagagcgacatcggcgtgaagttcgacactaggaacttcaggctgggctgggaccccgtgaccgagctggtcatgcccggggtgtacaccaacgaggctttccatcccgatattgtcttgctgcccggctgcggggtggacttcaccgagagccgcctcagcaacctgctgggcattcgcaagaggcagcccttccaggaaggcttccagatcatgtacgaggatctggaggggggcaacatccccgcgctcctggatgtcgacgcctatgagaaaagcaaggaggatgcagcagctgaagcaactgcagccgtagctaccgcctctaccgaggtcaggggcgataattttgcaagcgccgcagcagtggcagcggccgaggcggctgaaaccgaaagtaagatagtcattcagccggtggagaaggatagcaagaacaggagctacaacgtactaccggacaagataaacaccgcctaccgcagctggtacctagcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgcttacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcggtcgcgcaccaccgtcgacgacgtgatcgaccaggtggtggccgacgcgcgcaactacacccccgccgccgcgcccgtctccaccgtggacgccgtcatcgacagcgtggtggccgacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcttcaggcgccagcgccggcaggacccggagacgcgcggccacggcggcggcagcggccatcgccagcatgtcccgcccgcggcgagggaacgtgtactgggtgcgcgacgccgccaccggtgtgcgcgtgcccgtgcgcacccgcccccctcgcacttgaagatgttcacttcgcgatgttgatgtgtcccagcggcgaggaggatgtccaagcgcaaattcaaggaagagatgctccaggtcatcgcgcctgagatctacggccctgcggtggtgaaggaggaaagaaagccccgcaaaatcaagcgggtcaaaaaggacaaaaaggaagaagaaagtgatgtggacggattggtggagtttgtgcgcgagttcgccccccggcggcgcgtgcagtggcgcgggcggaaggtgcaaccggtgctgagacccggcaccaccgtggtcttcacgcccggcgagcgctccggcaccgcttccaagcgctcctacgacgaggtgtacggggatgatgatattctggagcaggcggccgagcgcctgggcgagtttgcttacggcaagcgcagccgttccgcaccgaaggaagaggcggtgtccatcccgctggaccacggcaaccccacgccgagcctcaagcccgtgaccttgcagcaggtgctgccgaccgcggcgccgcgccgggggttcaagcgcgagggcgaggatctgtaccccaccatgcagctgatggtgcccaagcgccagaagctggaagacgtgctggagaccatgaaggtggacccggacgtgcagcccgaggtcaaggtgcggcccatcaagcaggtggccccgggcctgggcgtgcagaccgtggacatcaagattcccacggagcccatggaaacgcagaccgagcccatgatcaagcccagcaccagcaccatggaggtgcagacggatccctggatgccatcggctcctagtcgaagaccccggcgcaagtacggcgcggccagcctgctgatgcccaactacgcgctgcatccttccatcatccccacgccgggctaccgcggcacgcgcttctaccgcggtcataccagcagccgccgccgcaagaccaccactcgccgccgccgtcgccgcaccgccgctgcaaccacccctgccgccctggtgcggagagtgtaccgccgcggccgcgcacctctgaccctgccgcgcgcgcgctaccacccgagcatcgccatttaaactttcgcctgctttgcagatcaatggccctcacatgccgccttcgcgttcccattacgggctaccgaggaagaaaaccgcgccgtagaaggctggcggggaacgggatgcgtcgccaccaccaccggcggcggcgcgccatcagcaagcggttggggggaggcttcctgcccgcgctgatccccatcatcgccgcggcgatcggggcgatccccggcattgcttccgtggcggtgcaggcctctcagcgccactgagacacacttggaaacatcttgtaataaaccaatggactctgacgctcctggtcctgtgatgtgttttcgtagacagatggaagacatcaatttttcgtccctggctccgcgacacggcacgcggccgttcatgggcacctggagcgacatcggcaccagccaactgaacgggggcgccttcaattggagcagtctctggagcgggcttaagaatttcgggtccacgcttaaaacctatggcagcaaggcgtggaacagcaccacagggcaggcgctgagggataagctgaaagagcagaacttccagcagaaggtggtcgatgggctcgcctcgggcatcaacggggtggtggacctggccaaccaggccgtgcagcggcagatcaacagccgcctggacccggtgccgcccgccggctccgtggagatgccgcaggtggaggaggagctgcctcccctggacaagcggggcgagaagcgaccccgccccgatgcggaggagacgctgctgacgcacacggacgagccgcccccgtacgaggaggcggtgaaactgggtctgcccaccacgcggcccatcgcgcccctggccaccggggtgctgaaacccgaaaagcccgcgaccctggacttgcctcctccccagccttcccgcccctctacagtggctaagcccctgccgccggtggccgtggcccgcgcgcgacccgggggcaccgcccgccctcatgcgaactggcagagcactctgaacagcatcgtgggtctgggagtgcagagtgtgaagcgccgccgctgctattaaacctaccgtagcgcttaacttgcttgtctgtgtgtgtatgtattatgtcgccgccgccgctgtccaccagaaggaggagtgaagaggcgcgtcgccgagttgcaagatggccaccccatcgatgctgccccagtgggcgtacatgcacatcgccggacaggacgcttcggagtacctgagtccgggtctggtgcagtttgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccctagcttcaaaccctactccggcaccgcctacaacagtctggcccccaagggagcacccaacacttgtcagtggacatataaagccgatggtgaaactgccacagaaaaaacctatacatatggaaatgcacccgtgcagggcattaacatcacaaaagatggtattcaacttggaactgacaccgatgatcagccaatctacgcagataaaacctatcagcctgaacctcaagtgggtgatgctgaatggcatgacatcactggtactgatgaaaagtatggaggcagagctcttaagcctgataccaaaatgaagccttgttatggttcttttgccaagcctactaataaagaaggaggtcaggcaaatgtgaaaacaggaacaggcactactaaagaatatgacatagacatggctttctttgacaacagaagtgcggctgctgctggcctagctccagaaattgttttgtatactgaaaatgtggatttggaaactccagatacccatattgtatacaaagcaggcacagatgacagcagctcttctattaatttgggtcagcaagccatgcccaacagacctaactacattggtttcagagacaactttatcgggctcatgtactacaacagcactggcaatatgggggtgctggccggtcaggcttctcagctgaatgctgtggttgacttgcaagacagaaacaccgagctgtcctaccagctcttgcttgactctctgggtgacagaacccggtatttcagtatgtggaatcaggcggtggacagctatgatcctgatgtgcgcattattgaaaatcatggtgtggaggatgaacttcccaactattgtttccctctggatgctgttggcagaacagatacttatcagggaattaaggctaatggaactgatcaaaccacatggaccaaagatgacagtgtcaatgatgctaatgagataggcaagggtaatccattcgccatggaaatcaacatccaagccaacctgtggaggaacttcctctacgccaacgtggccctgtacctgcccgactcttacaagtacacgccggccaatgttaccctgcccaccaacaccaacacctacgattacatgaacggccgggtggtggcgccctcgctggtggactcctacatcaacatcggggcgcgctggtcgctggatcccatggacaacgtgaaccccttcaaccaccaccgcaatgcggggctgcgctaccgctccatgctcctgggcaacgggcgctacgtgcccttccacatccaggtgccccagaaatttttcgccatcaagagcctcctgctcctgcccgggtcctacacctacgagtggaacttccgcaaggacgtcaacatgatcctgcagagctccctcggcaacgacctgcgcacggacggggcctccatctccttcaccagcatcaacctctacgccaccttcttccccatggcgcacaacacggcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggctggtccttcacgcgtctcaagaccaaggagacgccctcgctgggctccgggttcgacccctacttcgtctactcgggctccatcccctacctcgacggcaccttctacctcaaccacaccttcaagaaggtctccatcaccttcgactcctccgtcagctggcccggcaacgaccggctcctgacgcccaacgagttcgaaatcaagcgcaccgtcgacggcgagggctacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtggtggacgaggtcaactacaaggactaccaggccgtcaccctggcctaccagcacaacaactcgggcttcgtcggctacctcgcgcccaccatgcgccagggccagccctaccccgccaactacccctacccgctcatcggcaagagcgccgtcaccagcgtcacccagaaaaagttcctctgcgacagggtcatgtggcgcatccccttctccagcaacttcatgtccatgggcgcgctcaccgacctcggccagaacatgctctatgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacccccttctcggccggtaacgccaccacctaagctcttgcttcttgcaagccatggccgcgggctccggcgagcaggagctcagggccatcatccgcgacctgggctgcgggccctacttcctgggcaccttcgataagcgcttcccgggattcatggccccgcacaagctggcctgcgccatcgtcaacacggccggccgcgagaccgggggcgagcactggctggccttcgcctggaacccgcgctcgaacacctgctacctcttcgaccccttcgggttctcggacgagcgcctcaagcagatctaccagttcgagtacgagggcctgctgcgccgcagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaactcccactccgcctactttcgctcccaccgcgcgcgcatcgagaaggccaccgccttcgaccgcatgaatcaagacatgtaaaccgtgtgtgtatgttaaatgtctttaataaacagcactttcatgttacacatgcatctgagatgatttatttagaaatcgaaagggttctgccgggtctcggcatggcccgcgggcagggacacgttgcggaactggtacttggccagccacttgaactcggggatcagcagtttgggcagcggggtgtcggggaaggagtcggtccacagcttccgcgtcagttgcagggcgcccagcaggtcgggcgcggagatcttgaaatcgcagttgggacccgcgttctgcgcgcgggagttgcggtacacggggttgcagcactggaacaccatcagggccgggtgcttcacgctcgccagcaccgtcgcgtcggtgatgctctccacgtcgaggtcctcggcgttggccatcccgaagggggtcatcttgcaggtctgccttcccatggtgggcacgcacccgggcttgtggttgcaatcgcagtgcagggggatcagcatcatctgggcctggtcggcgttcatccccgggtacatggccttcatgaaagcctccaattgcctgaacgcctgctgggccttggctccctcggtgaagaagaccccgcaggacttgctagagaactggttggtggcgcacccggcgtcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcatgtgctccttctggatcatggtggtcccgtgcaggcaccgcagcttgccctcggcctcggtgcacccgtgcagccacagcgcgcacccggtgcactcccagttcttgtgggcgatctgggaatgcgcgtgcacgaagccctgcaggaagcggcccatcatggtggtcagggtcttgttgctagtgaaggtcagcggaatgccgcggtgctcctcgttgatgtacaggtggcagatgcggcggtacacctcgccctgctcgggcatcagctggaagttggctttcaggtcggtctccacgcggtagcggtccatcagcatagtcatgatttccatacccttctcccaggccgagacgatgggcaggctcatagggttcttcaccatcatcttagcgctagcagccgcggccagggggtcgctctcgtccagggtctcaaagctccgcttgccgtccttctcggtgatccgcaccggggggtagctgaagcccacggccgccagctcctcctcggcctgtctttcgtcctcgctgtcctggctgacgtcctgcaggaccacatgcttggtcttgcggggtttcttcttgggcggcagcggcggcggagatgttggagatggcgagggggagcgcgagttctcgctcaccactactatctcttcctcttcttggtccgaggccacgcggcggtaggtatgtctcttcgggggcagaggcggaggcgacgggctctcgccgccgcgacttggcggatggctggcagagccccttccgcgttcgggggtgcgctcccggcggcgctctgactgacttcctccgcggccggccattgtgttctcctagggaggaacaacaagcatggagactcagccatcgccaacctcgccatctgcccccaccgccgacgagaagcagcagcagcagaatgaaagcttaaccgccccgccgcccagccccgccacctccgacgcggccgtcccagacatgcaagagatggaggaatccatcgagattgacctgggctatgtgacgcccgcggagcacgaggaggagctggcagtgcgcttttcacaagaagagatacaccaagaacagccagagcaggaagcagagaatgagcagagtcaggctgggctcgagcatgacggcgactacctccacctgagcgggggggaggacgcgctcatcaagcatctggcccggcaggccaccatcgtcaaggatgcgctgctcgaccgcaccgaggtgcccctcagcgtggaggagctcagccgcgcctacgagttgaacctcttctcgccgcgcgtgccccccaagcgccagcccaatggcacctgcgagcccaacccgcgcctcaacttctacccggtcttcgcggtgcccgaggccctggccacctaccacatctttttcaagaaccaaaagatccccgtctcctgccgcgccaaccgcacccgcgccgacgcccttttcaacctgggtcccggcgcccgcctacctgatatcgcctccttggaagaggttcccaagatcttcgagggtctgggcagcgacgagactcgggccgcgaacgctctgcaaggagaaggaggagagcatgagcaccacagcgccctggtcgagttggaaggcgacaacgcgcggctggcggtgctcaaacgcacggtcgagctgacccatttcgcctacccggctctgaacctgccccccaaagtcatgagcgcggtcatggaccaggtgctcatcaagcgcgcgtcgcccatctccgaggacgagggcatgcaagactccgaggagggcaagcccgtggtcagcgacgagcagctggcccggtggctgggtcctaatgctagtccccagagtttggaagagcggcgcaaactcatgatggccgtggtcctggtgaccgtggagctggagtgcctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcttgcacgagaaccgcctggggcagaacgtgctgcacaccaccctgcgcggggaggcccggcgcgactacatccgcgactgcgtctacctctacctctgccacacctggcagacgggcatgggcgtgtggcagcagtgtctggaggagcagaacctgaaagagctctgcaagctcctgcagaagaacctcaagggtctgtggaccgggttcgacgagcgcaccaccgcctcggacctggccgacctcattttccccgagcgcctcaggctgacgctgcgcaacggcctgcccgactttatgagccaaagcatgttgcaaaactttcgctctttcatcctcgaacgctccggaatcctgcccgccacctgctccgcgctgccctcggacttcgtgccgctgaccttccgcgagtgccccccgccgctgtggagccactgctacctgctgcgcctggccaactacctggcctaccactcggacgtgatcgaggacgtcagcggcgagggcctgctcgagtgccactgccgctgcaacctctgcacgccgcaccgctccctggcctgcaacccccagctgctgagcgagacccagatcatcggcaccttcgagttgcaagggcccagcgaaggcgagggttcagccgccaaggggggtctgaaactcaccccggggctgtggacctcggcctacttgcgcaagttcgtgcccgaggactaccatcccttcgagatcaggttctacgaggaccaatcccatccgcccaaggccgagctgtcggcctgcgtcatcacccagggggcgatcctggcccaattgcaagccatccagaaatcccgccaagaattcttgctgaaaaagggccgcggggtctacctcgacccccagaccggtgaggagctcaaccccggcttcccccaggatgccccgaggaaacaagaagctgaaagtggagctgccgcccgtggaggatttggaggaagactgggagaacagcagtcaggcagaggaggaggagatggaggaagactgggacagcactcaggcagaggaggacagcctgcaagacagtctggaggaagacgaggaggaggcagaggaggaggtggaagaagcagccgccgccagaccgtcgtcctcggcgggggagaaagcaagcagcacggataccatctccgctccgggtcggggtcccgctcgaccacacagtagatgggacgagaccggacgattcccgaaccccaccacccagaccggtaagaaggagcggcagggatacaagtcctggcgggggcacaaaaacgccatcgtctcctgcttgcaggcctgcgggggcaacatctccttcacccggcgctacctgctcttccaccgcggggtgaactttccccgcaacatcttgcattactaccgtcacctccacagcccctactacttccaagaagaggcagcagcagcagaaaaagaccagcagaaaaccagcagctagaaaatccacagcggcggcagcaggtggactgaggatcgcggcgaacgagccggcgcaaacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccagtcgcagaaaaaggcgggaattacgtcacctgtgcccttcgccctagccgcctccacccatcatcatgagcaaagagattcccacgccttacatgtggagctaccagccccagatgggcctggccgccggtgccgcccaggactactccacccgcatgaattggctcagcgccgggcccgcgatgatctcacgggtgaatgacatccgcgcccaccgaaaccagatactcctagaacagtcagcgctcaccgccacgccccgcaatcacctcaatccgcgtaattggcccgccgccctggtgtaccaggaaattccccagcccacgaccgtactacttccgcgagacgcccaggccgaagtccagctgactaactcaggtgtccagctggcgggcggcgccaccctgtgtcgtcaccgccccgctcagggtataaagcggctggtgatccggggcagaggcacacagctcaacgacgaggtggtgagctcttcgctgggtctgcgacctgacggagtcttccaactcgccggatcggggagatcttccttcacgcctcgtcaggccgtcctgactttggagagttcgtcctcgcagccccgctcgggtggcatcggcactctccagttcgtggaggagttcactccctcggtctacttcaaccccttctccggctcccccggccactacccggacgagttcatcccgaacttcgacgccatcagcgagtcggtggacggctacgattgaaactaatcacccccttatccagtgaaataaagatcatattgatgatgattttacagaaataaaaaataatcatttgatttgaaataaagatacaatcatattgatgatttgagtttaacaaaaaaataaagaatcacttacttgaaatctgataccaggtctctgtccatgttttctgccaacaccacttcactcccctcttcccagctctggtactgcaggccccggcgggctgcaaacttcctccacacgctgaaggggatgtcaaattcctcctgtccctcaatcttcattttatcttctatcagatgtccaaaaagcgcgtccgggtggatgatgacttcgaccccgtctacccctacgatgcagacaacgcaccgaccgtgcccttcatcaacccccccttcgtctcttcagatggattccaagagaagcccctgggggtgttgtccctgcgactggccgaccccgtcaccaccaagaacggggaaatcaccctcaagctgggagagggggtggacctcgattcctcgggaaaactcatctccaacacggccaccaaggccgccgcccctctcagtttttccaacaacaccatttcccttaacatggatcaccccttttacactaaagatggaaaattatccttacaagtttctccaccattaaatatactgagaacaagcattctaaacacactagctttaggttttggatcaggtttaggactccgtggctctgccttggcagtacagttagtctctccacttacatttgatactgatggaaacataaagcttaccttagacagaggtttgcatgttacaacaggagatgcaattgaaagcaacataagctgggctaaaggtttaaaatttgaagatggagccatagcaaccaacattggaaatgggttagagtttggaagcagtagtacagaaacaggtgttgatgatgcttacccaatccaagttaaacttggatctggccttagctttgacagtacaggagccataatggctggtaacaaagaagacgataaactcactttgtggacaacacctgatccatcaccaaactgtcaaatactcgcagaaaatgatgcaaaactaacactttgcttgactaaatgtggtagtcaaatactggccactgtgtcagtcttagttgtaggaagtggaaacctaaaccccattactggcaccgtaagcagtgctcaggtgtttctacgttttgatgcaaacggtgttcttttaacagaacattctacactaaaaaaatactgggggtataggcagggagatagcatagatggcactccatataccaatgctgtaggattcatgcccaatttaaaagcttatccaaagtcacaaagttctactactaaaaataatatagtagggcaagtatacatgaatggagatgtttcaaaacctatgcttctcactataaccctcaatggtactgatgacagcaacagtacatattcaatgtcattttcatacacctggactaatggaagctatgttggagcaacatttggggctaactcttataccttctcatacatcgcccaagaatgaacactgtatcccaccctgcatgccaacccttcccaccccactctgtggaacaaactctgaaacacaaaataaaataaagttcaagtgttttattgattcaacagttttacaggattcgagcagttatttttcctccaccctcccaggacatggaatacaccaccctctccccccgcacagccttgaacatctgaatgccattggtgatggacatgcttttggtctccacgttccacacagtttcagagcgagccagtctcgggtcggtcagggagatgaaaccctccgggcactcccgcatctgcacctcacagctcaacagctgaggattgtcctcggtggtcgggatcacggttatctggaagaagcagaagagcggcggtgggaatcatagtccgcgaacgggatcggccggtggtgtcgcatcaggccccgcagcagtcgctgccgccgccgctccgtcaagctgctgctcagggggtccgggtccagggactccctcagcatgatgcccacggccctcagcatcagtcgtctggtgcggcgggcgcagcagcgcatgcggatctcgctcaggtcgctgcagtacgtgcaacacagaaccaccaggttgttcaacagtccatagttcaacacgctccagccgaaactcatcgcgggaaggatgctacccacgtggccgtcgtaccagatcctcaggtaaatcaagtggtgccccctccagaacacgctgcccacgtacatgatctccttgggcatgtggcggttcaccacctcccggtaccacatcaccctctggttgaacatgcagccccggatgatcctgcggaaccacagggccagcaccgccccgcccgccatgcagcgaagagaccccgggtcccggcaatggcaatggaggacccaccgctcgtacccgtggatcatctgggagctgaacaagtctatgttggcacagcacaggcatatgctcatgcatctcttcagcactctcaactcctcgggggtcaaaaccatatcccagggcacggggaactcttgcaggacagcgaaccccgcagaacagggcaatcctcgcacagaacttacattgtgcatggacagggtatcgcaatcaggcagcaccgggtgatcctccaccagagaagcgcgggtctcggtctcctcacagcgtggtaagggggccggccgatacgggtgatggcgggacgcggctgatcgtgttcgcgaccgtgtcatgatgcagttgctttcggacattttcgtacttgctgtagcagaacctggtccgggcgctgcacaccgatcgccggcggcggtctcggcgcttggaacgctcggtgttgaaattgtaaaacagccactctctcagaccgtgcagcagatctagggcctcaggagtgatgaagatcccatcatgcctgatggctctgatcacatcgaccaccgtggaatgggccagacccagccagatgatgcaattttgttgggtttcggtgacggcgggggagggaagaacaggaagaaccatgattaacttttaatccaaacggtctcggagtacttcaaaatgaagatcgcggagatggcacctctcgcccccgctgtgttggtggaaaataacagccaggtcaaaggtgatacggttctcgagatgttccacggtggcttccagcaaagcctccacgcgcacatccagaaacaagacaatagcgaaagcgggagggttctctaattcctcaatcatcatgttacactcctgcaccatccccagataattttcatttttccagccttgaatgattcgaactagttcctgaggtaaatccaagccagccatgataaagagctcgcgcagagcgccctccaccggcattcttaagcacaccctcataattccaagatattctgctcctggttcacctgcagcagattgacaagcggaatatcaaaatctctgccgcgatccctgagctcctccctcagcaataactgtaagtactctttcatatcctctccgaaatttttagccataggaccaccaggaataagattagggcaagccacagtacagataaaccgaagtcctccccagtgagcattgccaaatgcaagactgctataagcatgctggctagacccggtgatatcttccagataactggacagaaaatcgcccaggcaatttttaagaaaatcaacaaaagaaaaatcctccaggtggacgtttagagcctcgggaacaacgatgaagtaaatgcaagcggtgcgttccagcatggttagttagctgatctgtagaaaaaacaaaaatgaacattaaaccatgctagcctggcgaacaggtgggtaaatcgttctctccagcaccaggcaggccacggggtctccggcgcgaccctcgtaaaaattgtcgctatgattgaaaaccatcacagagagacgttcccggtggccggcgtgaatgattcgacaagatgaatacacccccggaacattggcgtccgcgagtgaaaaaaagcgcccgaggaagcaataaggcactacaatgctcagtctcaagtccagcaaagcgatgccatgcggatgaagcacaaaattctcaggtgcgtacaaaatgtaattactcccctcctgcacaggcagcaaagcccccgatccctccaggtacacatacaaagcctcagcgtccatagcttaccgagcagcagcacacaacaggcgcaagagtcagagaaaggctgagctctaacctgtccacccgctctctgctcaatatatagcccagatctacactgacgtaaaggccaaagtctaaaaatacccgccaaataatcacacacgcccagcacacgcccagaaaccggtgacacactcaaaaaaatacgcgcacttcctcaaacgcccaaaactgccgtcatttccgggttcccacgctacgtcatcaaaacacgactttcaaattccgtcgaccgttaaaaacgtcacccgccccgcccctaacggtcgcccgtctctcagccaatcagcgccccgcatccccaaattcaaacacctcatttgcatattaacgcgcacaaaaagtttgaggtatattattgatgatgg

SEQ ID NO: 14 - DNA, 8602 bp, synthetic construct
  misc_feature 1..8602: note = Description of Artificial Sequence: Synthetic polynucleotide
  source 1..8602: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgactctagaatagtctttaattaaagtccgccatatgaggccaccatgcagatcttcgtgaagaccctgaccggcaagaccatcaccctagaggtggagcccagtgacaccatcgagaacgtgaaggccaagatccaggataaagagggcatcccccctgaccagcagaggctgatctttgccggcaagcagctggaagatggccgcaccctctctgattacaacatccagaaggagtcaaccctgcacctggtccttcgcctgagaggtggcgctgcttacagtataatcaactttgaaaaactggctgcttacggcatcctgggctttgtgtttacactggctgcctacctgctgtttggctatcctgtgtacgtggccgcttatggactgtgtaccctggtggccatgctggctgcttacaatctggtgcctatggtggccacagtggccgcctattgtcttggcggactgctgacaatggtggcagcctacagcccgagctatgcgtatcatcagtttgcagcctacggcccaggaccaggcgctaaatttgtggctgcctggacactgaaagccgccgctggaccaggtcctggacagtacatcaaggccaacagcaagttcatcggcatcaccgaactcggcccaggaccaggctatccctacgatgtgcctgattacgcctgatagtgatgattcgaacggccgtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 15 - DNA, 9595 bp, synthetic construct
  misc_feature 1..9595: note = Description of Artificial Sequence: Synthetic polynucleotide
  source 1..9595: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgactctagaatagtctttaattaaagtccgccatatgagatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaattcgaacggccgtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 16 - protein, 139 aa, synthetic construct
  REGION 1..139: note = Description of Artificial Sequence: Synthetic polypeptide
PSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKV

SEQ ID NO: 17 - protein, 167 aa, synthetic construct
  REGION 1..167: note = Description of Artificial Sequence: Synthetic polypeptide
GVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVH

SEQ ID NO: 18 - protein, 10 aa, synthetic construct
  REGION 1..10: note = Description of Artificial Sequence: Synthetic peptide
GFTFSSYGMH

SEQ ID NO: 19 - protein, 15 aa, synthetic construct
  REGION 1..15: note = Description of Artificial Sequence: Synthetic peptide
VIWYDGSNKYYADSV

SEQ ID NO: 20 - protein, 16 aa, synthetic construct
  REGION 1..16: note = Description of Artificial Sequence: Synthetic peptide
DPRGATLYYYYYGMDV

SEQ ID NO: 21 - protein, 11 aa, synthetic construct
  REGION 1..11: note = Description of Artificial Sequence: Synthetic peptide
RASQSINSYLD

SEQ ID NO: 22 - protein, 7 aa, synthetic construct
  REGION 1..7: note = Description of Artificial Sequence: Synthetic peptide
AASSLQS

SEQ ID NO: 23 - protein, 9 aa, synthetic construct
  REGION 1..9: note = Description of Artificial Sequence: Synthetic peptide
QQYYSTPFT

SEQ ID NO: 24 - protein, 108 aa, synthetic construct
  REGION 1..108: note = Description of Artificial Sequence: Synthetic polypeptide
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 25 - protein, 121 aa, synthetic construct
  REGION 1..121: note = Description of Artificial Sequence: Synthetic polypeptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 26 - protein, 5 aa, synthetic construct
  REGION 1..5: note = Description of Artificial Sequence: Synthetic peptide
RYWMS

SEQ ID NO: 27 - protein, 17 aa, synthetic construct
  REGION 1..17: note = Description of Artificial Sequence: Synthetic peptide
NIKQDGSEKYYVDSVKG

SEQ ID NO: 28 - protein, 12 aa, synthetic construct
  REGION 1..12: note = Description of Artificial Sequence: Synthetic peptide
EGGWFGELAFDY

SEQ ID NO: 29 - protein, 12 aa, synthetic construct
  REGION 1..12: note = Description of Artificial Sequence: Synthetic peptide
RASQRVSSSYLA

SEQ ID NO: 30 - protein, 7 aa, synthetic construct
  REGION 1..7: note = Description of Artificial Sequence: Synthetic peptide
DASSRAT

SEQ ID NO: 31 - protein, 9 aa, synthetic construct
  REGION 1..9: note = Description of Artificial Sequence: Synthetic peptide
QQYGSLPWT

SEQ ID NO: 32 - DNA, 2019 bp, synthetic construct
  misc_feature 1..2019: note = Description of Artificial Sequence: Synthetic polynucleotide
  source 1..2019: mol_type = other DNA
gcccgggcatttaaatgcgatcgcatcgattacgactctagaatagtctagtccgcaggccaccatgcagatcttcgtgaagaccctgaccggcaagaccatcaccctagaggtggagcccagtgacaccatcgagaacgtgaaggccaagatccaggataaagagggcatcccccctgaccagcagaggctgatctttgccggcaagcagctggaagatggccgcaccctctctgattacaacatccagaaggagtcaaccctgcacctggtccttcgcctgagaggtgccatgtttcaggcgctgagcgaaggctgcaccccgtatgatattaaccagatgctgaacgtgctgggcgatcatcaggtctcaggccttgagcagcttgagagtataatcaactttgaaaaactgactgaatggaccagttctaatgttatgcctatcctgtctcctctgacaaagggcatcctgggcttcgtgtttaccctgaccgtgccttctgagagaggacttagctgcattagcgaagcggatgcgaccaccccggaaagcgcgaacctgggcgaagaaattctgagccagctgtatctttggccaagggtgacctaccattcccctagttatgcttaccaccaatttgaaagacgagccaaatataaaagacacttccccggctttggccagagcctgctgtttggctaccctgtgtacgtgttcggcgattgcgtgcagggcgattgggatgcgattcgctttcgctattgcgcgccgccgggctatgcgctgctgcgctgcaacgataccaactatagcgctctgctggctgtgggggccctagaaggacccaggaatcaggactggcttggtgtcccaagacaacttgtaactcggatgcaggctattcagaatgccggcctgtgtaccctggtggccatgctggaagagacaatcttctggctgcaagcgtttctgatggcgctgaccgatagcggcccgaaaaccaacattattgtggatagccagtatgtgatgggcattagcaaaccgagctttcaggaatttgtggattgggaaaacgtgagcccggaactgaacagcaccgatcagccgttttggcaagccggaatcctggccagaaatctggtgcctatggtggccacagtgcagggccagaacctgaagtaccagggtcagtcactagtcatctctgcttctatcattgtcttcaacctgctggaactggaaggtgattatcgagatgatggcaacgtgtgggtgcataccccgctgagcccgcgcaccctgaacgcgtgggtgaaagcggtggaagaaaaaaaaggtattccagttcacctagagctggccagtatgaccaacatggagctcatgagcagtattgtgcatcagcaggtcagaacatacggccccgtgttcatgtgtctcggcggactgcttacaatggtggctggtgctgtgtggctgacagtgcgagtgctcgagctgttccgggccgcgcagctggccaacgacgtggtcctccagatcatggagctttgtggtgcagcgtttcgccaggtgtgccataccaccgtgccgtggccgaacgcgagcctgaccccgaaatggaacaacgaaaccacccagccccagatcgccaactgcagcgtgtatgacttttttgtgtggctccattattattctgttcgagacacactttggccaagggtgacctaccatatgaacaaatatgcgtatcatatgctggaaagacgagccaaatataaaagaggaccaggacctggcgctaaatttgtggccgcctggacactgaaagccgctgctggtcctggacctggccagtacatcaaggccaacagcaagttcatcggcatcaccgaactcggacccggaccaggctgatgatttcgaaatttaaataagcttgcggccgctagggataacagggtaattatcacgcccaaacatttacagccgcggtgtcaaaaaccgcgtgg

SEQ ID NO: 33 - protein, 619 aa, synthetic construct
  REGION 1..619: note = Description of Artificial Sequence: Synthetic polypeptide
MQIFVKTLTGKTITLEVEPSDTIENVKAKIQDKEGIPPDQQRLIFAGKQLEDGRTLSDYNIQKESTLHLVLRLRGAMFQALSEGCTPYDINQMLNVLGDHQVSGLEQLESIINFEKLTEWTSSNVMPILSPLTKGILGFVFTLTVPSERGLSCISEADATTPESANLGEEILSQLYLWPRVTYHSPSYAYHQFERRAKYKRHFPGFGQSLLFGYPVYVFGDCVQGDWDAIRFRYCAPPGYALLRCNDTNYSALLAVGALEGPRNQDWLGVPRQLVTRMQAIQNAGLCTLVAMLEETIFWLQAFLMALTDSGPKTNIIVDSQYVMGISKPSFQEFVDWENVSPELNSTDQPFWQAGILARNLVPMVATVQGQNLKYQGQSLVISASIIVFNLLELEGDYRDDGNVWVHTPLSPRTLNAWVKAVEEKKGIPVHLELASMTNMELMSSIVHQQVRTYGPVFMCLGGLLTMVAGAVWLTVRVLELFRAAQLANDVVLQIMELCGAAFRQVCHTTVPWPNASLTPKWNNETTQPQIANCSVYDFFVWLHYYSVRDTLWPRVTYHMNKYAYHMLERRAKYKRGPGPGAKFVAAWTLKAAAGPGPGQYIKANSKFIGITELGPGPG

SEQ ID NO: 34 - DNA, 1638 bp, synthetic construct
  misc_feature 1..1638: note = Description of Artificial Sequence: Synthetic polynucleotide
  source 1..1638: mol_type = other DNA
atggccgggatgttccaggcactgtccgaaggctgcacaccctatgatattaaccagatgctgaatgtcctgggagaccaccaggtctctggcctggagcagctggagagcatcatcaacttcgagaagctgaccgagtggacaagctccaatgtgatgcctatcctgtccccactgaccaagggcatcctgggcttcgtgtttaccctgacagtgccttctgagcggggcctgtcttgcatcagcgaggcagacgcaaccacaccagagtccgccaatctgggcgaggagatcctgtctcagctgtacctgtggccccgggtgacatatcactccccttcttacgcctatcaccagttcgagcggagagccaagtacaagagacacttcccaggctttggccagtctctgctgttcggctaccccgtgtacgtgttcggcgattgcgtgcagggcgactgggatgccatccggtttagatactgcgcaccacctggatatgcactgctgaggtgtaacgacaccaattattccgccctgctggcagtgggcgccctggagggccctcgcaatcaggattggctgggcgtgccaaggcagctggtgacacgcatgcaggccatccagaacgcaggcctgtgcaccctggtggcaatgctggaggagacaatcttctggctgcaggcctttctgatggccctgaccgacagcggccccaagacaaacatcatcgtggattcccagtacgtgatgggcatctccaagccttctttccaggagtttgtggactgggagaacgtgagcccagagctgaattccaccgatcagccattctggcaggcaggaatcctggcaaggaacctggtgcctatggtggccacagtgcagggccagaatctgaagtaccagggccagagcctggtcatcagcgcctccatcatcgtgtttaacctgctggagctggagggcgactatcgggacgatggcaacgtgtgggtgcacaccccactgagccccagaacactgaacgcctgggtgaaggccgtggaggagaagaagggcatcccagtgcacctggagctggcctccatgaccaatatggagctgatgtctagcatcgtgcaccagcaggtgaggacatacggacccgtgttcatgtgcctgggaggcctgctgaccatggtggcaggagccgtgtggctgacagtgcgggtgctggagctgttcagagccgcccagctggccaacgatgtggtgctgcagatcatggagctgtgcggagcagcctttcgccaggtgtgccacaccacagtgccatggcccaatgcctccctgacccccaagtggaacaatgagacaacacagcctcagatcgccaactgtagcgtgtacgacttcttcgtgtggctgcactactatagcgtgagggataccctgtggccccgcgtgacataccacatgaataagtacgcctatcacatgctggagaggcgcgccaagtataagagaggccctggcccaggcgcaaagtttgtggcagcatggaccctgaaggccgccgccggccccggccccggccagtatatcaaggctaacagtaagttcattggaatcacagagctgggacccggacctgga

SEQ ID NO: 35 - protein, 546 aa, synthetic construct
  REGION 1..546: note = Description of Artificial Sequence: Synthetic polypeptide
MAGMFQALSEGCTPYDINQMLNVLGDHQVSGLEQLESIINFEKLTEWTSSNVMPILSPLTKGILGFVFTLTVPSERGLSCISEADATTPESANLGEEILSQLYLWPRVTYHSPSYAYHQFERRAKYKRHFPGFGQSLLFGYPVYVFGDCVQGDWDAIRFRYCAPPGYALLRCNDTNYSALLAVGALEGPRNQDWLGVPRQLVTRMQAIQNAGLCTLVAMLEETIFWLQAFLMALTDSGPKTNIIVDSQYVMGISKPSFQEFVDWENVSPELNSTDQPFWQAGILARNLVPMVATVQGQNLKYQGQSLVISASIIVFNLLELEGDYRDDGNVWVHTPLSPRTLNAWVKAVEEKKGIPVHLELASMTNMELMSSIVHQQVRTYGPVFMCLGGLLTMVAGAVWLTVRVLELFRAAQLANDVVLQIMELCGAAFRQVCHTTVPWPNASLTPKWNNETTQPQIANCSVYDFFVWLHYYSVRDTLWPRVTYHMNKYAYHMLERRAKYKRGPGPGAKFVAAWTLKAAAGPGPGQYIKANSKFIGITELGPGPG

SEQ ID NO: 36 - DNA, 2019 bp, synthetic construct
  misc_feature 1..2019: note = Description of Artificial Sequence: Synthetic polynucleotide
  source 1..2019: mol_type = other DNA
gcccgggcatttaaatgcgatcgcatcgattacgactctagaatagtctagtccgcaggccaccatgcagatcttcgtgaagaccctgaccggcaagaccatcaccctagaggtggagcccagtgacaccatcgagaacgtgaaggccaagatccaggataaagagggcatcccccctgaccagcagaggctgatctttgccggcaagcagctggaagatggccgcaccctctctgattacaacatccagaaggagtcaaccctgcacctggtccttcgcctgagaggtgccatgtttcaggcgctgagcgaaggctgcaccccgtatgatattaaccagatgctgaacgtgctgggcgatcatcagtttaagcacatcaaagcctttgaccggacatttgctaacaacccaggtcccatggttgtgtttgccacacctgggcctatcctgtctcctctgacaaagggcatcctgggcttcgtgtttaccctgaccgtgccttctgagagaggacttagctgcattagcgaagcggatgcgaccaccccggaaagcgcgaacctgggcgaagaaattctgagccagctgtatctttggccaagggtgacctaccattcccctagttatgcttaccaccaatttgaaagacgagccaaatataaaagacacttccccggctttggccagagcctgctgtttggctaccctgtgtacgtgttcggcgattgcgtgcagggcgattgggatgcgattcgctttcgctattgcgcgccgccgggctatgcgctgctgcgctgcaacgataccaactatagcgctctgctggctgtgggggccctagaaggacccaggaatcaggactggcttggtgtcccaagacaacttgtaactcggatgcaggctattcagaatgccggcctgtgtaccctggtggccatgctggaagagacaatcttctggctgcaagcgtttctgatggcgctgaccgatagcggcccgaaaaccaacattattgtggatagccagtatgtgatgggcattagcaaaccgagctttcaggaatttgtggattgggaaaacgtgagcccggaactgaacagcaccgatcagccgttttggcaagccggaatcctggccagaaatctggtgcctatggtggccacagtgcagggccagaacctgaagtaccagggtcagtcactagtcatctctgcttctatcattgtcttcaacctgctggaactggaaggtgattatcgagatgatggcaacgtgtgggtgcataccccgctgagcccgcgcaccctgaacgcgtgggtgaaagcggtggaagaaaaaaaaggtattccagttcacctagagctggccagtatgaccaacatggagctcatgagcagtattgtgcatcagcaggtcagaacatacggccccgtgttcatgtgtctcggcggactgcttacaatggtggctggtgctgtgtggctgacagtgcgagtgctcgagctgttccgggccgcgcagctggccaacgacgtggtcctccagatcatggagctttgtggtgcagcgtttcgccaggtgtgccataccaccgtgccgtggccgaacgcgagcctgaccccgaaatggaacaacgaaaccacccagccccagatcgccaactgcagcgtgtatgacttttttgtgtggctccattattattctgttcgagacacactttggccaagggtgacctaccatatgaacaaatatgcgtatcatatgctggaaagacgagccaaatataaaagaggaccaggacctggcgctaaatttgtggccgcctggacactgaaagccgctgctggtcctggacctggccagtacatcaaggccaacagcaagttcatcggcatcaccgaactcggacccggaccaggctgatgatttcgaaatttaaataagcttgcggccgctagggataacagggtaattatcacgcccaaacatttacagccgcggtgtcaaaaaccgcgtgg

SEQ ID NO: 37 - protein, 619 aa, synthetic construct
  REGION 1..619: note = Description of Artificial Sequence: Synthetic polypeptide
MQIFVKTLTGKTITLEVEPSDTIENVKAKIQDKEGIPPDQQRLIFAGKQLEDGRTLSDYNIQKESTLHLVLRLRGAMFQALSEGCTPYDINQMLNVLGDHQFKHIKAFDRTFANNPGPMVVFATPGPILSPLTKGILGFVFTLTVPSERGLSCISEADATTPESANLGEEILSQLYLWPRVTYHSPSYAYHQFERRAKYKRHFPGFGQSLLFGYPVYVFGDCVQGDWDAIRFRYCAPPGYALLRCNDTNYSALLAVGALEGPRNQDWLGVPRQLVTRMQAIQNAGLCTLVAMLEETIFWLQAFLMALTDSGPKTNIIVDSQYVMGISKPSFQEFVDWENVSPELNSTDQPFWQAGILARNLVPMVATVQGQNLKYQGQSLVISASIIVFNLLELEGDYRDDGNVWVHTPLSPRTLNAWVKAVEEKKGIPVHLELASMTNMELMSSIVHQQVRTYGPVFMCLGGLLTMVAGAVWLTVRVLELFRAAQLANDVVLQIMELCGAAFRQVCHTTVPWPNASLTPKWNNETTQPQIANCSVYDFFVWLHYYSVRDTLWPRVTYHMNKYAYHMLERRAKYKRGPGPGAKFVAAWTLKAAAGPGPGQYIKANSKFIGITELGPGPG

SEQ ID NO: 38 - DNA, 228 bp, synthetic construct
  misc_feature 1..228: note = Description of Artificial Sequence: Synthetic polynucleotide
  source 1..228: mol_type = other DNA
atgcagatcttcgtgaagaccctgaccggcaagaccatcaccctagaggtggagcccagtgacaccatcgagaacgtgaaggccaagatccaggataaagagggcatcccccctgaccagcagaggctgatctttgccggcaagcagctggaagatggccgcaccctctctgattacaacatccagaaggagtcaaccctgcacctggtccttcgcctgagaggtggc

SEQ ID NO: 39 - DNA, 228 bp, synthetic construct
  misc_feature 1..228: note = Description of Artificial Sequence: Synthetic polynucleotide
  source 1..228: mol_type = other DNA
atgcagatcttcgtgaagaccctgaccggcaagaccatcaccctagaggtggagcccagtgacaccatcgagaacgtgaaggccaagatccaggataaagagggcatcccccctgaccagcagaggctgatctttgccggcaagcagctggaagatggccgcaccctctctgattacaacatccagaaggagtcaaccctgcacctggtccttcgcctgagaggtgcc

SEQ ID NO: 40 - DNA, 78 bp, Homo sapiens
  source 1..78: mol_type = other DNA
atggccgtcatggcgccccgaaccctcgtcctgctactctcgggggctctggccctgacccagacctgggcgggctct

SEQ ID NO: 41 - DNA, 201 bp, Homo sapiens
  source 1..201: mol_type = other DNA
ccgtcttcccagcccaccatccccatcgtgggcatcattgctggcctggttctctttggagctgtgatcactggagctgtggtcgctgctgtgatgtggaggaggaagagctcagatagaaaaggagggagctactctcaggctgcaagcagtgacagtgcccagggctctgatgtgtctctcacagcttgtaaagtgtga

SEQ ID NO: 42 - DNA, 60 bp, synthetic construct
  misc_feature 1..60: note = Description of Artificial Sequence: Synthetic oligonucleotide
  source 1..60: mol_type = other DNA
atggagaccgatacactgctgctgtgggtgctgctcctgtgggtgccaggaagcacaggc

SEQ ID NO: 43 - DNA, 3178 bp, Homo sapiens
  source 1..3178: mol_type = other DNA
ggcaccgattcggggcctgcccggacttcgccgcacgctgcagaacctcgcccagcgcccaccatgccccggcagctcagcgcggcggccgcgctcttcgcgtccctggccgtaattttgcacgatggcagtcaaatgagagcaaaagcatttccagaaaccagagattattctcaacctactgcagcagcaacagtacaggacataaaaaaacctgtccagcaaccagctaagcaagcacctcaccaaactttagcagcaagattcatggatggtcatatcacctttcaaacagcggccacagtaaaaattccaacaactaccccagcaactacaaaaaacactgcaaccaccagcccaattacctacaccctggtcacaacccaggccacacccaacaactcacacacagctcctccagttactgaagttacagtcggccctagcttagccccttattcactgccacccaccatcaccccaccagctcatacagctggaaccagttcatcaaccgtcagccacacaactgggaacaccactcaacccagtaaccagaccacccttccagcaactttatcgatagcactgcacaaaagcacaaccggtcagaagcctgatcaacccacccatgccccaggaacaacggcagctgcccacaataccacccgcacagctgcacctgcctccacggttcctgggcccacccttgcacctcagccatcgtcagtcaagactggaatttatcaggttctaaacggaagcagactctgtataaaagcagagatggggatacagctgattgttcaagacaaggagtcggttttttcacctcggagatacttcaacatcgaccccaacgcaacgcaagcctctgggaactgtggcacccgaaaatccaaccttctgttgaattttcagggcggatttgtgaatctcacatttaccaaggatgaagaatcatattatatcagtgaagtgggagcctatttgaccgtctcagatccagagacagtttaccaaggaatcaaacatgcggtggtgatgttccagacagcagtcgggcattccttcaagtgcgtgagtgaacagagcctccagttgtcagcccacctgcaggtgaaaacaaccgatgtccaacttcaagcctttgattttgaagatgaccactttggaaatgtggatgagtgctcgtctgactacacaattgtgcttcctgtgattggggccatcgtggttggtctctgccttatgggtatgggtgtctataaaatccgcctaaggtgtcaatcatctggataccagagaatctaattgttgcccggggggaatgaaaataatggaatttagagaactctttcatcccttccaggatggatgttgggaaattccctcagagtgtgggtccttcaaacaatgtaaaccaccatcttctattcaaatgaagtgagtcatgtgtgatttaagttcaggcagcacatcaatttctaaatactttttgtttattttatgaaagatatagtgagctgtttattttctagtttcctttagaatattttagccactcaaagtcaacatttgagatatgttgaattaacataatatatgtaaagtagaataagccttcaaattataaaccaagggtcaattgtaactaatactactgtgtgtgcattgaagattttattttacccttgatcttaacaaagcctttgctttgttatcaaatggactttcagtgcttttactatctgtgttttatggtttcatgtaacatacatattcctggtgtagcacttaactccttttccactttaaatttgtttttgttttttgagacggagtttcactcttgtcacccaggctggagtacagtggcacgatctcggcttatggcaacctccgcctcccgggttcaagtgattctcctgcttcagcttcccgagtagctgggattacaggcacacactaccacgcctggctaatttttgtatttttattatagacgggtttcaccatgttggccagactggtcttgaactcttgacctcaggtgatccacccacctcagcctcccaaagtgctgggattacaggcatgagccattgcgcccggccttaaatgttttttttaatcatcaaaaagaacaacatatctcaggttgtctaagtgtttttatgtaaaaccaacaaaaagaacaaatcagcttatattttttatcttgatgactcctgctccagaattgctagactaagaattaggtggctacagatggtagaactaaacaataagcaagagacaataataatggcccttaattattaacaaagtgccagagtctaggctaagcactttatctatatctcatttcattctcacaacttataagtgaatgagtaaactgagacttaagggaactgaatcacttaaatgtcacctggctaactgatggcagagccagagcttgaattcatgttggtctgacatcaaggtctttggtcttctccctacaccaagttacctacaagaacaatgacaccacactctgcctgaaggctcacacctcataccagcatacgctcaccttacagggaaatgggtttatccaggatcatgagacattagggtagatgaaaggagagctttgcagataacaaaatagcctatccttaataaatcctccactctctggaaggagactgaggggctttgtaaaacattagtcagttgctcatttttatgggattgcttagctgggctgtaaagatgaaggcatcaaataaactcaaagtatttttaaatttttttgataatagagaaacttcgctaaccaactgttctttcttgagtgtatagccccatcttgtggtaacttgctgcttctgcacttcatatccatatttcctattgttcactttattctgtagagcagcctgccaagaattttatttctgctgttttttttgctgctaaagaaaggaactaagtcaggatgttaacagaaaagtccacataaccctagaattcttagtcaaggaataattcaagtcagcctagagaccatgttgactttcctcatgtgtttccttatgactcagtaagttggcaaggtcctgactttagtcttaataaaacattgaattgtagtaaaggtttttgcaataaaaacttactttgg

SEQ ID NO: 44 - DNA, 1858 bp, Mus sp.
  source 1..1858: mol_type = other DNA
attccggaggtgaaaaacaatggcacaacgtgtataatggccagcttctctgcctcctttctgaccacctacgagactgcgaatggttctcagatcgtgaacatttccctgccagcctctgcagaagtactgaaaaatggcagttcttgtggtaaagaaaatgtttctgaccccagcctcacaattacttttggaagaggatatttactgacactcaacttcacaaaaaatacaacacgttacagtgtccagcatatgtattttacatataacttgtcagatacagaacattttcccaatgccatcagcaaagagatctacaccatggattccacaactgacatcaaggcagacatcaacaaagcataccggtgtgtcagtgatatccgggtctacatgaagaatgtgaccgttgtgctccgggatgccactatccaggcctacctgtcgagtggcaacttcagcaaggaagagacacactgcacacaggatggaccttccccaaccactgggccacccagcccctcaccaccacttgtgcccacaaaccccactgtatccaagtacaatgttactggtaacaacggaacctgcctgctggcctctatggcactgcaactgaatatcacctacctgaaaaaggacaacaagacggtgaccagagcgttcaacatcagcccaaatgacacatctagtgggagttgcggtatcaacttggtgaccctgaaagtggagaacaagaacagagccctggaattgcagtttgggatgaatgccagctctagcctgtttttcttgcaaggagtgcgcttgaatatgactcttcctgatgccctagtgcccacattcagcatctccaaccattcactgaaagctcttcaggccactgtgggaaactcatacaagtgcaacactgaggaacacatctttgtcagcaagatgctctccctcaatgtcttcagtgtgcaggtccaggctttcaaggtggacagtgacaggtttgggtctgtggaagagtgtgttcaggatggtaacaacatgttgatccccattgctgtgggcggtgccctggcagggctgatcctcatcgtcctcattgcctacctcattggcaggaagaggagtcacgccggctatcagaccatctagcctggtgggcaggtgcaccagagatgcacaggggcctgttctcacatccccaagcttagataggtgtggaagggaggcacactttctggcaaactgttttaaaatctgctttatcaaatgtgaagttcatcttgcaacatttactatgcacaaaggaataactattgaaatgacggtgttaattttgctaactgggttaaatattgatgagaaggctccactgatttgacttttaagacttggtgtttggttcttcattcttttactcagatttaagcctatcaaagggatactctggtccagaccttggcctggcaagggtggctgatggttaggctgcacacacttaagaagcaacgggagcagggaaggcttgcacacaggcacgcacagggtcaacctctggacacttggcttgggctacctggccttgggggggctgaactctggcatctggctgggtacacacccccccaatttctgtgctctgccacccgtgagctgccactttcctaaatagaaaatggcattatttttatttacttttttgtaaagtgatttccagtcttgtgttggcgttcagggtggccctgtctctgcactgtgtacaataatagattcacactgctgacgtgtcttgcagcgtaggtgggttgtacactgggcatcagctcacgtaatgcattgcctgtaacgatgctaataaaaa

SEQ ID NO: 45 - DNA, 2339 bp, Homo sapiens
  source 1..2339: mol_type = other DNA
ggcccaaccgccgcccgcgcccccgctctccgcaccgtacccggccgcctcgcgccatggcggcccccggcagcgcccggcgacccctgctgctgctactgctgttgctgctgctcggcctcatgcattgtgcgtcagcagcaatgtttatggtgaaaaatggcaacgggaccgcgtgcataatggccaacttctctgctgccttctcagtgaactacgacaccaagagtggccctaagaacatgacctttgacctgccatcagatgccacagtggtgctcaaccgcagctcctgtggaaaagagaacacttctgaccccagtctcgtgattgcttttggaagaggacatacactcactctcaatttcacgagaaatgcaacacgttacagcgtccagctcatgagttttgtttataacttgtcagacacacaccttttccccaatgcgagctccaaagaaatcaagactgtggaatctataactgacatcagggcagatatagataaaaaatacagatgtgttagtggcacccaggtccacatgaacaacgtgaccgtaacgctccatgatgccaccatccaggcgtacctttccaacagcagcttcagcaggggagagacacgctgtgaacaagacaggccttccccaaccacagcgccccctgcgccacccagcccctcgccctcacccgtgcccaagagcccctctgtggacaagtacaacgtgagcggcaccaacgggacctgcctgctggccagcatggggctgcagctgaacctcacctatgagaggaaggacaacacgacggtgacaaggcttctcaacatcaaccccaacaagacctcggccagcgggagctgcggcgcccacctggtgactctggagctgcacagcgagggcaccaccgtcctgctcttccagttcgggatgaatgcaagttctagccggtttttcctacaaggaatccagttgaatacaattcttcctgacgccagagaccctgcctttaaagctgccaacggctccctgcgagcgctgcaggccacagtcggcaattcctacaagtgcaacgcggaggagcacgtccgtgtcacgaaggcgttttcagtcaatatattcaaagtgtgggtccaggctttcaaggtggaaggtggccagtttggctctgtggaggagtgtctgctggacgagaacagcatgctgatccccatcgctgtgggtggtgccctggcggggctggtcctcatcgtcctcatcgcctacctcgtcggcaggaagaggagtcacgcaggctaccagactatctagcctggtgcacgcaggcacagcagctgcaggggcctctgttcctttctctgggcttagggtcctgtcgaaggggaggcacactttctggcaaacgtttctcaaatctgcttcatccaatgtgaagttcatcttgcagcatttactatgcacaacagagtaactatcgaaatgacggtgttaattttgctaactgggttaaatattttgctaactggttaaacattaatatttaccaaagtaggattttgagggtgggggtgctctctctgagggggtgggggtgccgctgtctctgaggggtgggggtgccgctgtctctgaggggtgggggtgccgctctctctgagggggtgggggtgccgctttctctgagggggtgggggtgccgctctctctgagggggtgggggtgctgctctctccgaggggtggaatgccgctgtctctgaggggtgggggtgccgctctaaattggctccatatcatttgagtttagggttctggtgtttggtttcttcattctttactgcactcagatttaagccttacaaagggaaagcctctggccgtcacacgtaggacgcatgaaggtcactcgtggtgaggctgacatgctcacacattacaacagtagagagggaaaatcctaagacagaggaactccagagatgagtgtctggagcgcttcagttcagctttaaaggccaggacgggccacacgtggctggcggcctcgttccagtggcggcacgtccttgggcgtctctaatgtctgcagctcaagggctggcacttttttaaatataaaaatgggtgttatttttatttttttttgtaaagtgatttttggtcttctgttgacattcggggtgatcctgttctgcgctgtgtacaatgtgagatcggtgcgttctcctgatgttttgccgtggcttggggattgtacacgggaccagctcacgtaatgcattgcctgtaacaatgtaataaaaagcctctttcttttaaaaaaaaaaaaaaaaaaaaaaaa

SEQ ID NO: 46 - DNA, 45 bp, synthetic construct
  misc_feature 1..45: note = Description of Artificial Sequence: Synthetic oligonucleotide
  source 1..45: mol_type = other DNA
cagtacatcaaggccaacagcaagttcatcggcatcaccgaactc

SEQ ID NO: 47 - protein, 15 aa, synthetic construct
  REGION 1..15: note = Description of Artificial Sequence: Synthetic peptide
QYIKANSKFIGITEL

SEQ ID NO: 48 - DNA, 39 bp, synthetic construct
  misc_feature 1..39: note = Description of Artificial Sequence: Synthetic oligonucleotide
  source 1..39: mol_type = other DNA
gctaaatttgtggctgcctggacactgaaagccgccgct

SEQ ID NO: 49 - protein, 13 aa, synthetic construct
  REGION 1..13: note = Description of Artificial Sequence: Synthetic peptide
AKFVAAWTLKAAA

SEQ ID NO: 50 - DNA, 593 bp, Woodchuck hepatitis virus
  source 1..593: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgt

SEQ ID NO: 51 - DNA, 589 bp, synthetic construct
  misc_feature 1..589: note = Description of Artificial Sequence: Synthetic polynucleotide
  source 1..589: mol_type = other DNA
tctcccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatg

SEQ ID NO: 52 - DNA, 720 bp, synthetic construct
  misc_feature 1..720: note = Description of Artificial Sequence: Synthetic polynucleotide
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtag

SEQ ID NO: 53 - DNA, 1563 bp, synthetic construct
  misc_feature 1..1563: note = Description of Artificial Sequence: Synthetic polynucleotide
  source 1..1563: mol_type = other DNA
atgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatgataa

SEQ ID NO: 54 - DNA, 1653 bp, synthetic construct
  misc_feature 1..1653: note = Description of Artificial Sequence: Synthetic polynucleotide
  source 1..1653: mol_type = other DNA
atggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaa

SEQ ID NO: 55 - DNA, 66 bp, Foot-and-mouth disease virus
  source 1..66: mol_type = other DNA
gtaaagcaaacactgaactttgaccttctcaagttggctggagacgttgagtccaatcctgggccc

SEQ ID NO: 56 - protein, 5 aa, synthetic construct
  REGION 1..5: note = Description of Artificial Sequence: Synthetic peptide
GPGPG

SEQ ID NO: 57 - protein, 25 aa, synthetic construct
  REGION 1..25: note = Description of Artificial Sequence: Synthetic peptide
GQSLVISASIIVFNLLELEGDYRDD

SEQ ID NO: 58 - protein, 25 aa, synthetic construct
  REGION 1..25: note = Description of Artificial Sequence: Synthetic peptide
GIPVHLELASMTNMELMSSIVHQQV

SEQ ID NO: 59 - protein, 25 aa, synthetic construct
  REGION 1..25: note = Description of Artificial Sequence: Synthetic peptide
RVLELFRAAQLANDVVLQIMELCGA

SEQ ID NO: 60 - protein, 25 aa, synthetic construct
  REGION 1..25: note = Description of Artificial Sequence: Synthetic peptide
ALLAVGALEGPRNQDWLGVPRQLVT

SEQ ID NO: 61 - protein, 25 aa, synthetic construct
  REGION 1..25: note = Description of Artificial Sequence: Synthetic peptide
VTNTEMFVTAPDNLGYMYEVQWPGQ

SEQ ID NO: 62 - protein, 25 aa, synthetic construct
  REGION 1..25: note = Description of Artificial Sequence: Synthetic peptide
TQPQIANCSVYDFFVWLHYYSVRDT

SEQ ID NO: 63 - protein, 543 aa, synthetic construct
  REGION 1..543: note = Description of Artificial Sequence: Synthetic polypeptide
MFQALSEGCTPYDINQMLNVLGDHQVSGLEQLESIINFEKLTEWTSSNVMPILSPLTKGILGFVFTLTVPSERGLSCISEADATTPESANLGEEILSQLYLWPRVTYHSPSYAYHQFERRAKYKRHFPGFGQSLLFGYPVYVFGDCVQGDWDAIRFRYCAPPGYALLRCNDTNYSALLAVGALEGPRNQDWLGVPRQLVTRMQAIQNAGLCTLVAMLEETIFWLQAFLMALTDSGPKTNIIVDSQYVMGISKPSFQEFVDWENVSPELNSTDQPFWQAGILARNLVPMVATVQGQNLKYQGQSLVISASIIVFNLLELEGDYRDDGNVWVHTPLSPRTLNAWVKAVEEKKGIPVHLELASMTNMELMSSIVHQQVRTYGPVFMCLGGLLTMVAGAVWLTVRVLELFRAAQLANDVVLQIMELCGAAFRQVCHTTVPWPNASLTPKWNNETTQPQIANCSVYDFFVWLHYYSVRDTLWPRVTYHMNKYAYHMLERRAKYKRGPGPGAKFVAAWTLKAAAGPGPGQYIKANSKFIGITELGPGPG

SEQ ID NO: 64 - protein, 543 aa, synthetic construct
  REGION 1..543: note = Description of Artificial Sequence: Synthetic polypeptide
GQSLVISASIIVFNLLELEGDYRDDGIPVHLELASMTNMELMSSIVHQQVRVLELFRAAQLANDVVLQIMELCGAGQSLVISASIIVFNLLELEGDYRDDGIPVHLELASMTNMELMSSIVHQQVRVLELFRAAQLANDVVLQIMELCGAGQSLVISASIIVFNLLELEGDYRDDGIPVHLELASMTNMELMSSIVHQQVRVLELFRAAQLANDVVLQIMELCGAGQSLVISASIIVFNLLELEGDYRDDGIPVHLELASMTNMELMSSIVHQQVRVLELFRAAQLANDVVLQIMELCGAGQSLVISASIIVFNLLELEGDYRDDGIPVHLELASMTNMELMSSIVHQQVRVLELFRAAQLANDVVLQIMELCGAGQSLVISASIIVFNLLELEGDYRDDGIPVHLELASMTNMELMSSIVHQQVRVLELFRAAQLANDVVLQIMELCGAGQSLVISASIIVFNLLELEGDYRDDGIPVHLELASMTNMELMSSIVHQQVGPGPGAKFVAAWTLKAAAGPGPGQYIKANSKFIGITELGPGPG

SEQ ID NO: 65 - protein, 418 aa, synthetic construct
  REGION 1..418: note = Description of Artificial Sequence: Synthetic polypeptide
GQSLVISASIIVFNLLELEGDYRDDGIPVHLELASMTNMELMSSIVHQQVRVLELFRAAQLANDVVLQIMELCGAGQSLVISASIIVFNLLELEGDYRDDGIPVHLELASMTNMELMSSIVHQQVRVLELFRAAQLANDVVLQIMELCGAGQSLVISASIIVFNLLELEGDYRDDGIPVHLELASMTNMELMSSIVHQQVRVLELFRAAQLANDVVLQIMELCGAGQSLVISASIIVFNLLELEGDYRDDGIPVHLELASMTNMELMSSIVHQQVRVLELFRAAQLANDVVLQIMELCGAGQSLVISASIIVFNLLELEGDYRDDGIPVHLELASMTNMELMSSIVHQQVRVLELFRAAQLANDVVLQIMELCGAGPGPGAKFVAAWTLKAAAGPGPGQYIKANSKFIGITELGPGPG

SEQ ID NO: 66 - protein, 543 aa, synthetic construct
  REGION 1..543: note = Description of Artificial Sequence: Synthetic polypeptide
ALLAVGALEGPRNQDWLGVPRQLVTVTNTEMFVTAPDNLGYMYEVQWPGQTQPQIANCSVYDFFVWLHYYSVRDTGQSLVISASIIVFNLLELEGDYRDDGIPVHLELASMTNMELMSSIVHQQVRVLELFRAAQLANDVVLQIMELCGANEIPFTYEQLSIFKHKLDKTYPQGYSRASLLGPGFVLIWIPALLPALRLSNWHKGWNWSSGHNECPVGASCHPFTKTLLKVSKGQKMNAQAIALVACYLRMFQALSEGCTPYDINQMLNVLGDHQSCISEADATTPESANLGEEILSQLYWDAIRFRYCAPPGYALLRCNDTNYSAFLMALTDSGPKTNIIVDSQYVMGIGNVWVHTPLSPRTLNAWVKAVEEKKAFRQVCHTTVPWPNASLTPKWNNETWQAGILARNLVPMVATVQGQNLKYQRTYGPVFMCLGGLLTMVAGAVWLTVHFPGFGQSLLFGYPVYVFGDCVQGDPILSPLTKGILGFVFTLTVPSERGLGPGPGAKFVAAWTLKAAAGPGPGQYIKANSKFIGITELGPGPG

SEQ ID NO: 67 - protein, 543 aa, synthetic construct
  REGION 1..543: note = Description of Artificial Sequence: Synthetic polypeptide
ALLAVGALEGPRNQDWLGVPRQLVTVTNTEMFVTAPDNLGYMYEVQWPGQTQPQIANCSVYDFFVWLHYYSVRDTGQSLVISASIIVFNLLELEGDYRDDGIPVHLELASMTNMELMSSIVHQQVRVLELFRAAQLANDVVLQIMELCGANEIPFTYEQLSIFKHKLDKTYPQGYSRASLLGPGFVLIWIPALLPALRLSNWHKGWNWSSGHNECPVGASCHPFTKTLLKVSKGQKMNAQAIALVACYLRALLAVGALEGPRNQDWLGVPRQLVTVTNTEMFVTAPDNLGYMYEVQWPGQTQPQIANCSVYDFFVWLHYYSVRDTGQSLVISASIIVFNLLELEGDYRDDGIPVHLELASMTNMELMSSIVHQQVRVLELFRAAQLANDVVLQIMELCGANEIPFTYEQLSIFKHKLDKTYPQGYSRASLLGPGFVLIWIPALLPALRLSNWHKGWNWSSGHNECPVGASCHPFTKTLLKVSKGQKMNAQAIALVACYLRGPGPGAKFVAAWTLKAAAGPGPGQYIKANSKFIGITELGPGPG

SEQ ID NO: 68 - protein, 559 aa, synthetic construct
  REGION 1..559: note = Description of Artificial Sequence: Synthetic polypeptide
ALLAVGALEGPRNQDWLGVPRQLVTVTNTEMFVTAPDNLGYMYEVQWPGQTQPQIANCSVYDFFVWLHYYSVRDTPSKPSFQEFVDWENVSPELNSTDQPFLEFKHIKAFDRTFANNPGPMVVFATPGMALLAVGALEGPRNQDWLGVPRQLVTVTNTEMFVTAPDNLGYMYEVQWPGQTQPQIANCSVYDFFVWLHYYSVRDTPSKPSFQEFVDWENVSPELNSTDQPFLEFKHIKAFDRTFANNPGPMVVFATPGMALLAVGALEGPRNQDWLGVPRQLVTVTNTEMFVTAPDNLGYMYEVQWPGQTQPQIANCSVYDFFVWLHYYSVRDTPSKPSFQEFVDWENVSPELNSTDQPFLEFKHIKAFDRTFANNPGPMVVFATPGMALLAVGALEGPRNQDWLGVPRQLVTVTNTEMFVTAPDNLGYMYEVQWPGQTQPQIANCSVYDFFVWLHYYSVRDTPSKPSFQEFVDWENVSPELNSTDQPFLEFKHIKAFDRTFANNPGPMVVFATPGMGPGPGAKFVAAWTLKAAAGPGPGQYIKANSKFIGITELGPGPG

SEQ ID NO: 69 - protein, 543 aa, synthetic construct
  REGION 1..543: note = Description of Artificial Sequence: Synthetic polypeptide
ALLAVGALEGPRNQDWLGVPRQLVTVTNTEMFVTAPDNLGYMYEVQWPGQTQPQIANCSVYDFFVWLHYYSVRDTALLAVGALEGPRNQDWLGVPRQLVTVTNTEMFVTAPDNLGYMYEVQWPGQTQPQIANCSVYDFFVWLHYYSVRDTALLAVGALEGPRNQDWLGVPRQLVTVTNTEMFVTAPDNLGYMYEVQWPGQTQPQIANCSVYDFFVWLHYYSVRDTALLAVGALEGPRNQDWLGVPRQLVTVTNTEMFVTAPDNLGYMYEVQWPGQTQPQIANCSVYDFFVWLHYYSVRDTALLAVGALEGPRNQDWLGVPRQLVTVTNTEMFVTAPDNLGYMYEVQWPGQTQPQIANCSVYDFFVWLHYYSVRDTALLAVGALEGPRNQDWLGVPRQLVTVTNTEMFVTAPDNLGYMYEVQWPGQTQPQIANCSVYDFFVWLHYYSVRDTALLAVGALEGPRNQDWLGVPRQLVTVTNTEMFVTAPDNLGYMYEVQWPGQGPGPGAKFVAAWTLKAAAGPGPGQYIKANSKFIGITELGPGPG

SEQ ID NO: 70 - protein, 118 aa, synthetic construct
  REGION 1..118: note = Description of Artificial Sequence: Synthetic polypeptide
GQSLVISASIIVFNLLELEGDYRDDGIPVHLELASMTNMELMSSIVHQQVRVLELFRAAQLANDVVLQIMELCGAGPGPGAKFVAAWTLKAAAGPGPGQYIKANSKFIGITELGPGPG

SEQ ID NO: 71 - protein, 118 aa, synthetic construct
  REGION 1..118: note = Description of Artificial Sequence: Synthetic polypeptide
ALLAVGALEGPRNQDWLGVPRQLVTVTNTEMFVTAPDNLGYMYEVQWPGQTQPQIANCSVYDFFVWLHYYSVRDTGPGPGAKFVAAWTLKAAAGPGPGQYIKANSKFIGITELGPGPG

SEQ ID NO: 72 - protein, 100 aa, synthetic construct
  REGION 1..100: note = Description of Artificial Sequence: Synthetic polypeptide
VTNTEMFVTAPDNLGYMYEVQWPGQTQPQIANCSVYDFFVWLHYYSVRDTVTNTEMFVTAPDNLGYMYEVQWPGQTQPQIANCSVYDFFVWLHYYSVRDT

SEQ ID NO: 73 - protein, 10 aa, synthetic construct
  REGION 1..10: note = Description of Artificial Sequence: Synthetic peptide
YVYVADVAAK

SEQ ID NO: 74 - DNA, 17 bp, synthetic construct
  misc_feature 1..17: note = Description of Artificial Sequence: Synthetic oligonucleotide
  source 1..17: mol_type = other DNA
taatacgactcactata

SEQ ID NO: 75 - DNA, 7952 bp, synthetic construct
  misc_feature 1..7952: note = Description of Artificial Sequence: Synthetic polynucleotide
  source 1..7952: mol_type = other DNA
taatacgactcactataatgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacttatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa